(19) United States
(12) Patent Application Publication
(10) Pub. No.: US 2025/0223582 A1
(43) Pub. Date: Jul. 10, 2025

(54) ENGINEERED RNA LIGASES

(71) Applicant: Codexis, Inc., Redwood City, CA (US)

(72) Inventors: Oscar Alvizo, Fremont, CA (US); David Elgart, San Mateo, CA (US); Ravi David Garcia, Los Gatos, CA (US); Nikki D. Kruse, San Carlos, CA (US); Mathew G. Miller, San Carlos, CA (US); Jovana Nazor, Milpitas, CA (US); Zhe Rui, Newark, CA (US); Arya Srivastava, Foster City, CA (US)

(21) Appl. No.: 19/009,695

(22) Filed: Jan. 3, 2025

Related U.S. Application Data
(60) Provisional application No. 63/618,203, filed on Jan. 5, 2024.
(60) Provisional application No. 63/554,938, filed on Feb. 16, 2024.
(60) Provisional application No. 63/646,753, filed on May 13, 2024.
(60) Provisional application No. 63/718,750, filed on Nov. 11, 2024.

(51) Int. Cl. C12N 9/00

(52) U.S. Cl. CPC C12N 9/93; C12Y 605/01003

(57) ABSTRACT

The present disclosure relates to engineered RNA ligase polypeptides and compositions thereof, as well as polynucleotides encoding the engineered RNA ligase polypeptides. The present disclosure also provides methods of using the engineered RNA ligase polypeptides or compositions thereof for molecular biological, diagnostic, and other purposes.

### CROSS-REFERENCE TO RELATED APPLICATIONS

This application claims the benefit of U.S. Provisional Application 63/618,203, filed Jan. 5, 2024, U.S. Provisional Application 63/554,938, filed Feb. 16, 2024, U.S. Provisional Application 63/646,753, filed May 13, 2024, and U.S. Provisional Application 63/718,750, filed Nov. 11, 2024, all of which are incorporated by reference herein.

### REFERENCE TO SEQUENCE LISTING, TABLE OR COMPUTER PROGRAM

The Sequence Listing concurrently submitted herewith as file name CX10-262WO5_ST26.xml, created on Jan. 3, 2025, with a file size of 6,025,556 bytes, is part of the specification and is incorporated by reference herein.

### TECHNICAL FIELD

The present disclosure provides engineered RNA ligase polypeptides and compositions thereof, and polynucleotides encoding the engineered RNA ligase polypeptides. The disclosure further provides methods of using the recombinant RNA ligase or compositions thereof for the synthesis of polynucleotides and as molecular biological tools.

### BACKGROUND

Oligonucleotide therapeutics, either as synthetic single stranded or double stranded polynucleotides, represent a class of therapeutics that exert their biological effects by modulating gene expression. The oligonucleotides are designed primarily to target pre-mRNA, mRNA, or non-coding RNA (e.g., miRNA) to promote RNA degradation, modulate splicing, interrupt translation, or in some instances activate expression. Small interfering RNAs (siRNA) are a class of oligonucleotide therapeutics that are typically double stranded oligonucleotides that act via RISC (RNA-induced silencing complex) pathway, where the strand complementary to an RNA target, also referred to as the guide strand, targets the RNA for degradation or translation inhibition. To improve in vivo stability, the oligonucleotides have modifications at the 2′-position of the sugar residue, such as 2′-O-methyl, 2′-O-ethyl, 2′-O-methoxyethyl, or 2′-fluoro, and contain at the 5′- and 3′-ends non-natural internucleoside linkages, such as a phosphorothioate linkages. Conjugating targeting moieties to the oligonucleotide, such as GalNac and lipid groups, can enhance delivery to cells and tissues.

Modified oligonucleotides are generally synthesized chemically, such as by solid-phase synthesis using phosphoramidite chemistry. However, chemical synthesis has several disadvantages, including low efficiency in synthesis of long oligonucleotides, difficulty in scale-up of manufacturing, and toxic chemical waste and solvent consumption, e.g., toluene and acetonitrile. To circumvent some of the limitations in chemical synthesis, RNA ligase 2 can be used to ligate shorter oligonucleotides to generate the oligonucleotide therapeutic. Double stranded RNA ligases, also referred to as RNA ligase 2 or RNA ligase II, preferentially join a nick in an RNA duplex. In view of the potential for use of RNA ligase 2 for the synthesis of modified oligonucleotides, desirable are RNA ligases that exhibit increased efficiency and promiscuity in ligating polynucleotides that contain nucleotide analogs, such as modified sugar residues, modified nucleobases, and non-standard internucleoside linkages.

### SUMMARY

The present disclosure provides engineered RNA ligase polypeptides and compositions thereof, as well as polynucleotides encoding the engineered RNA ligase polypeptides. The present disclosure also provides methods of using the engineered RNA ligase polypeptides and compositions thereof for nucleic acid synthesis, diagnostic assays, and other purposes.

In one aspect, the present disclosure provides an engineered RNA ligase, or a functional fragment thereof, comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to a reference sequence corresponding to residues 12 to 346 of an even-numbered SEQ ID NO. of SEQ ID NOS: 2-1470 and 1478-3058, or to a reference sequence corresponding to an even-numbered SEQ ID NO. of SEQ ID NOs: 2-1470 and 1478-3058, wherein the amino acid sequence comprises one or more substitutions relative to a reference sequence corresponding to residues 12 to 346 of SEQ ID NO: 2, 54, 244, 648, 862, 956, 1088, 1416, 1612, 1840, 1866, 1954, 2062, 2674, 2754, or 2786, or relative to a reference sequence corresponding to SEQ ID NO: 2, 54, 244, 648, 862, 956, 1088, 1416, 1612, 1840, 1866, 1954, 2062, 2674, 2754, or 2786.

In some embodiments, the engineered RNA ligase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12 to 346 of SEQ ID NO: 2, 54, 244, 648, 862, 956, 1088, 1416, 1612, 1840, 1866, 1954, 2062, 2674, 2754, or 2786, or to the reference sequence corresponding to SEQ ID NO: 2, 54, 244, 648, 862, 956, 1088, 1416, 1612, 1840, 1866, 1954, 2062, 2674, 2754, or 2786, wherein the amino acid sequence comprises one or more substitutions relative to the reference sequence corresponding to residues 12 to 346 of SEQ ID NO: 2, 54, 244, 648, 862, 956, 1088, 1416, 1612, 1840, 1866, 1954, 2062, 2674, 2754, or 2786, or relative to the reference sequence corresponding to SEQ ID NO: 2, 54, 244, 648, 862, 956, 1088, 1416, 1612, 1840, 1866, 1954, 2062, 2674, 2754, or 2786.

In some embodiments, the engineered RNA ligase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12 to 346 of SEQ ID NO: 2, or to the reference sequence corresponding to SEQ ID NO: 2, wherein the amino acid sequence comprises one or more substitutions relative to the reference sequence corresponding to residues 12 to 346 of SEQ ID NO: 2, or relative to the reference sequence corresponding to SEQ ID NO: 2.

In some embodiments, the engineered RNA ligase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12 to 346 of SEQ ID NO: 54, 244, 648, 862, or 956, or to the reference sequence corresponding to SEQ ID NO: 54, 244, 648, 862, 956, 1088, 1416, 1612, 1840, 1866, 1954, 2062, 2674, 2754, or 2786, wherein the amino acid sequence comprises one or more substitutions relative to the reference sequence corresponding to residues 12 to 346 of SEQ ID NO: 2, or relative to the reference sequence corresponding to SEQ ID NO: 2.

In some embodiments, the engineered RNA ligase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12 to 346 of an even-numbered SEQ ID NO. of SEQ ID NOs: 4-1470 and 1478-3058, or to the reference sequence corresponding to an even-numbered SEQ ID NO. of SEQ ID NOs: 4-1470 and 1478-3058, wherein the amino acid sequence comprises one or more substitutions relative to the reference sequence corresponding to residues 12 to 346 of SEQ ID NO: 2, or relative to the reference sequence corresponding to SEQ ID NO: 2.

In some embodiments, the amino acid sequence of the engineered RNA ligase comprises at least a substitution at amino acid position 10, 11, 12, 13, 15, 17, 18, 21, 23, 25, 26, 28, 29, 30, 32, 33, 37, 38, 39, 40, 41, 42, 45, 52, 54, 56, 57, 58, 60, 61, 62, 64, 65, 67, 71, 73, 74, 75, 77, 80, 81, 83, 84, 86, 87, 89, 90, 91, 93, 95, 97, 99, 100, 101, 102, 103, 104, 105, 106, 107, 109, 113, 114, 116, 117, 119, 121, 122, 123, 125, 126, 127, 128, 129, 131, 135, 137, 140, 141, 143, 144, 148, 150, 154, 156, 157, 158, 159, 160, 161, 162, 165, 169, 170, 171, 173, 175, 177, 178, 179, 181, 183, 184, 185, 190, 191, 192, 193, 194, 197, 198, 204, 206, 208, 210, 212, 213, 214, 215, 218, 223, 224, 226, 228, 229, 230, 232, 236, 238, 241, 243, 247, 248, 249, 250, 251, 252, 253, 254, 255, 256, 257, 258, 260, 261, 264, 265, 266, 267, 268, 270, 271, 272, 274, 276, 277, 280, 282, 283, 286, 288, 289, 290, 291, 292, 294, 295, 296, 297, 298, 299, 300, 301, 302, 305, 306, 307, 310, 311, 315, 316, 317, 318, 323, 324, 325, 326, 327, 330, 331, 332, 334, 335, 336, 337, 338, 341, 342, 344, 345, or 346, or any combinations thereof, wherein the amino acid positions are relative to the reference sequence corresponding to residues 12 to 346 of SEQ ID NO: 2, or relative to the reference sequence corresponding to SEQ ID NO: 2.

In some embodiments, the amino acid sequence of the engineered RNA ligase comprises at least a substitution at amino acid position 10, 11, 12, 13, 17, 21, 23, 25, 26, 28, 29, 30, 32, 37, 38, 40, 41, 42, 45, 52, 54, 56, 57, 58, 60, 61, 62, 64, 65, 67, 71, 73, 74, 75, 77, 80, 83, 84, 87, 90, 91, 93, 97, 99, 100, 101, 102, 103, 104, 105, 106, 107, 109, 113, 114, 121, 122, 123, 125, 126, 127, 128, 129, 131, 135, 137, 140, 141, 143, 148, 150, 157, 158, 160, 161, 162, 165, 169, 170, 173, 178, 179, 184, 190, 191, 192, 193, 194, 197, 198, 204, 206, 208, 210, 213, 215, 218, 223, 228, 229, 232, 236, 238, 241, 243, 248, 249, 250, 251, 252, 253, 254, 255, 258, 261, 264, 265, 267, 268, 270, 271, 272, 274, 276, 277, 282, 286, 289, 290, 292, 294, 295, 297, 298, 299, 300, 301, 302, 305, 311, 318, 323, 324, 325, 327, 332, 336, 338, 341, 344, or 346, or a substitution 18A/H, 33A/I/T/V, 39P, 86D/L, 89C/D/E/L/N/Q/R/S/T/V/Y, 95A/C/G/I, 116L, 117A/Q, 119L, 144L, 154S/V, 156G/H/S, 171S, 175A, 177K, 181E/N/P/R/S, 183L, 224A/Q/R, 212E/G/K/L, 226R, 230T/V, 247I/L/R/S, 256E/G/M/S/W, 257D/E/G, 260G, 280L/W, 283C/V, 288L/R/W, 291S, 296E/G/L/Q/W, 306G/T, 307K/R, 310H/L/M/N/Q/R/S/T, 315E, 316I/Y, 317E/F/G/Q/V/W, 326C/L, 330A/D/E/G/K, 331D/G/S, 334E, 335Q, 337A/V, 338G/L/M, 342R/S/T, 345Q/S; or any combinations thereof, wherein the amino acid positions are relative to the reference sequence corresponding to residues 12 to 346 of SEQ ID NO: 2, or relative to the reference sequence corresponding to SEQ ID NO: 2.

In some embodiments, the amino acid sequence of the engineered RNA ligase comprises at least a substitution at amino acid position 11, 12, 23, 25, 29, 37, 38, 75, 86, 89, 93, 101, 122, 143, 157, 183, 192, 194, 198, 212, 214, 230, 241, 248, 249, 250, 251, 254, 266, 268, 280, 291, 295, 298, 307, 316, 318, 326, 330, or 331, or any combinations thereof, wherein the amino acid positions are relative to the reference sequence corresponding to residues 12 to 346 of SEQ ID NO: 2, or relative to the reference sequence corresponding to SEQ ID NO: 2.

In some embodiments, the amino acid sequence of the engineered RNA ligase comprises at least a substitution at amino acid position 11, 12, 23, 25, 29, 37, 38, 75, 93, 101, 122, 143, 157, 192, 194, 198, 241, 248, 249, 250, 251, 254, 268, 295, 298, or 318; or substitution 86D/L, 89C/D/E/L/N/Q/R/S/T/V/Y, 183L, 212E/G/K/L, 214A/C/H/K/L/Q/Y, 226R, 230T/V, 266C/I, 280L/W, 291H/L/Q/R/S/T/W, 307K/R, 326C/L, 330A/D/E/G/K, or 331D/G/S; or any combinations thereof, wherein the amino acid positions are relative to the reference sequence corresponding to residues 12 to 346 of SEQ ID NO: 2, or relative to the reference sequence corresponding to SEQ ID NO: 2.

In some embodiments, the amino acid sequence of the engineered RNA ligase further comprises at least a substitution at amino acid position 2, 14, 15, 63, 69, 81, 85, 95, 138, 142, 149, 159, 171, 186, 195, 202, 266, 275, 283, 285, 303, 314, 333, or 339; a substitution 18I, 33L, 39Q/S, 86G, 89G/H/I/M/W, 116N, 117D, 119T, 144W, 154C, 156A/L/N/Q/T, 175K, 177P, 181D, 183V, 185G/K, 2121, 2141, 224Y, 2261, 230D, 247K, 256A, 257L, 260S, 280G/N, 288E, 291P, 296K, 306L, 307E/Q, 310K, 315S/T, 316L, 317A, 326R, 330M/R, 331L/R/W, 334R, 335D/H, 337L, 3421, or 345V; or any combinations thereof, wherein the amino acid positions are relative to the reference sequence corresponding to residues 12 to 346 of SEQ ID NO: 2, or relative to the reference sequence corresponding to SEQ ID NO: 2.

In some embodiments, the amino acid sequence of the engineered RNA ligase comprises at least a substitution or substitution set at amino acid position(s) 256, 258, 254, 251, 248, 37, 54, 232, 15, 71, 26, 116, 38, 25, 106/251, 52, 33, 247, 37/249/266/316/330/334/335, 21, 11/37/249/266/316, 261, 11/266/316, 11/248/249/266, 331, 11/248/249, 255, 37/249/266/316/334, 266, 11/37/156/249/335, 37/156/248/249/266/334, 334, 156, 11/266/316/334, 11/39/156/248/316, 11/249, 37/156,107/251, 156/249/316, 37/156/316, 11/37/156/249, 11/37/334, 194, 37/316,175, 11/156/249/316, 11/37, 252, 12, 37/266, 11/37/156/266, 11/37/156/248/266, 37/236/248/249, 37/156/248/249, 11/266/330/335, 249, 11/156/248/249/316, 37/249/316, 11, 11/156, 11/37/249, 330, 316, 156/249/266/335, 249/266/316/330/335, 11/156/249, 266/316/330/334/335, 156/248/249/316/334, 37/156/248/249/316, 156/266, 248/249, 156/249/316/330/335, 218, 37/156/248/249/266, 11/37/156/248/249/316, 37/156/316/330, 11/37/248/249/316/330, 11/37/248/249, 249/330/335, 11/37/156/330/334, 40, 11/37/148/249/316, 83, 323, 193, 335, or 257, wherein the amino acid positions are relative to the reference sequence corresponding to residues 12 to 346 of SEQ ID NO: 2, or relative to the reference sequence corresponding to SEQ ID NO: 2.

In some embodiments, the amino acid sequence of the engineered RNA ligase comprises at least one substitution provided in Tables 5.1, 6.1, 6.2, 7.1, 8.1, 8.2, 9.1, 10.1, 11.1, 12.1, 13.1, 14.1, 15.1, 16.1, 17.1, 18.1, 19.1, 20.1, 21.1, 22.1, 23.1, 24.1, 25.1, and 26.1, wherein the amino acid positions are relative to the reference sequence corresponding to residues 12 to 346 of SEQ ID NO: 2, or relative to the reference sequence corresponding to SEQ ID NO: 2.

In some embodiments, the amino acid sequence of the engineered RNA ligase comprises at least a substitution or substitution set of an RNA ligase variant provided in Tables 5.1, 6.1, 6.2, 7.1, 8.1, 8.2, 9.1, 10.1, 11.1, 12.1, 13.1, 14.1, 15.1, 16.1, 17.1, 18.1, 19.1, 20.1, 21.1, 22.1, 23.1, 24.1, 25.1, and 26.1, wherein the amino acid positions are relative to the reference sequence corresponding to residues 12 to 346 of SEQ ID NO: 2, or relative to the reference sequence corresponding to SEQ ID NO: 2.

In some embodiments, the engineered RNA ligase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to a reference sequence comprising a substitution or substitution set provided in Tables 5.1, 6.1, 6.2, 7.1, 8.1, 8.2, 9.1, 10.1, 11.1, 12.1, 13.1, 14.1, 15.1, 16.1, 17.1, 18.1, 19.1, 20.1, 21.1, 22.1, 23.1, 24.1, 25.1, and 26.1, wherein the amino acid positions are relative to the reference sequence corresponding to residues 12 to 346 of SEQ ID NO: 2, or relative to the reference sequence corresponding to SEQ ID NO: 2.

In some embodiments, the engineered RNA ligase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12 to 346 of SEQ ID NO: 54, 244, 648, 862, 956, 1088, 1416, 1612, 1840, 1866, 1954, 2062, 2674, 2754, or 2786, or to the reference sequence corresponding to SEQ ID NO: 54, 244, 648, 862, 956, 1088, 1416, 1612, 1840, 1866, 1954, 2062, 2674, 2754, or 2786.

In some embodiments, the engineered RNA ligase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12 to 346 of an even-numbered SEQ ID NO. of SEQ ID NOs: 4-1470 and 1478-3058, or to the reference sequence corresponding to an even-numbered SEQ ID NO. of SEQ ID NOs: 4-1470 and 1478-3058.

In some embodiments, the engineered RNA ligase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12 to 346 of SEQ ID NO: 54, 244, 648, 862, 956, 1088, 1416, 1612, 1840, 1866, 1954, 2062, 2674, 2754, or 2786, or to the reference sequence corresponding to SEQ ID NO: 54, 244, 648, 862, 956, 1088, 1416, 1612, 1840, 1866, 1954, 2062, 2674, 2754, or 2786, wherein the amino acid sequence comprises one or more substitutions relative to the reference sequence corresponding to residues 12 to 346 of SEQ ID NO: 54, 244, 648, 862, 956, 1088, 1416, 1612, 1840, 1866, 1954, 2062, 2674, 2754, or 2786, or relative to the reference sequence corresponding to SEQ ID NO: 54, 244, 648, 862, 956, 1088, 1416, 1612, 1840, 1866, 1954, 2062, 2674, 2754, or 2786.

In some embodiments, the engineered RNA ligase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12 to 346 of an even-numbered SEQ ID NO. of SEQ ID NOs: 4-1470 and 1478-3058, or to the reference sequence corresponding to an even-numbered SEQ ID NO. of SEQ ID NOs: 4-1470 and 1478-3058, wherein the amino acid sequence comprises one or more substitutions relative to the reference sequence corresponding to residues 12 to 346 of SEQ ID NO: 54, 244, 648, 862, 956, 1088, 1416, 1612, 1840, 1866, 1954, 2062, 2674, 2754, or 2786, or relative to the reference sequence corresponding to SEQ ID NO: 54, 244, 648, 862, 956, 1088, 1416, 1612, 1840, 1866, 1954, 2062, 2674, 2754, or 2786.

In some embodiments, the amino acid sequence of the engineered RNA ligase comprises at least a substitution at amino acid position 10, 11, 12, 13, 15, 17, 18, 21, 23, 25, 26, 28, 29, 30, 32, 33, 37, 38, 39, 40, 41, 42, 45, 52, 54, 56, 57, 58, 60, 61, 62, 64, 65, 67, 71, 73, 74, 75, 77, 80, 81, 83, 84, 86, 87, 89, 90, 91, 93, 95, 97, 99, 100, 101, 102, 103, 104, 105, 106, 107, 109, 113, 114, 116, 117, 119, 121, 122, 123, 125, 126, 127, 128, 129, 131, 135, 137, 140, 141, 143, 144, 148, 150, 154, 156, 157, 158, 159, 160, 161, 162, 165, 169, 170, 171, 173, 175, 177, 178, 179, 181, 183, 184, 185, 190, 191, 192, 193, 194, 197, 198, 204, 206, 208, 210, 212, 213, 214, 215, 218, 223, 224, 226, 228, 229, 230, 232, 236, 238, 241, 243, 247, 248, 249, 250, 251, 252, 253, 254, 255, 256, 257, 258, 260, 261, 264, 265, 266, 267, 268, 270, 271, 272, 274, 276, 277, 280, 282, 283, 286, 288, 289, 290, 291, 292, 294, 295, 296, 297, 298, 299, 300, 301, 302, 305, 306, 307, 310, 311, 315, 316, 317, 318, 323, 324, 325, 326, 327, 330, 331, 332, 334, 335, 336, 337, 338, 341, 342, 344, 345, or 346, or any combinations thereof, wherein the amino acid positions are relative to the reference sequence corresponding to residues 12 to 346 of SEQ ID NO: 54, 244, 648, 862, 956, 1088, 1416, 1612, 1840, 1866, 1954, 2062, 2674, 2754, or 2786, or relative to the reference sequence corresponding to SEQ ID NO: 54, 244, 648, 862, 956, 1088, 1416, 1612, 1840, 1866, 1954, 2062, 2674, 2754, or 2786.

In some embodiments, the amino acid sequence of the engineered RNA ligase comprises at least a substitution at amino acid position 10, 11, 12, 13, 17, 21, 23, 25, 26, 28, 29, 30, 32, 37, 38, 40, 41, 42, 45, 52, 54, 56, 57, 58, 60, 61, 62, 64, 65, 67, 71, 73, 74, 75, 77, 80, 83, 84, 87, 90, 91, 93, 97, 99, 100, 101, 102, 103, 104, 105, 106, 107, 109, 113, 114, 121, 122, 123, 125, 126, 127, 128, 129, 131, 135, 137, 140, 141, 143, 148, 150, 157, 158, 160, 161, 162, 165, 169, 170, 173, 178, 179, 184, 190, 191, 192, 193, 194, 197, 198, 204, 206, 208, 210, 213, 215, 218, 223, 228, 229, 232, 236, 238, 241, 243, 248, 249, 250, 251, 252, 253, 254, 255, 258, 261, 264, 265, 267, 268, 270, 271, 272, 274, 276, 277, 282, 286, 289, 290, 292, 294, 295, 297, 298, 299, 300, 301, 302, 305, 311, 318, 323, 324, 325, 327, 332, 336, 338, 341, 344, or 346; a substitution or amino acid residue 18A/H, 33A/I/T/V, 39P, 86D/L, 89C/D/E/L/N/Q/R/S/T/V/Y, 95A/C/G/I, 116L, 117A/Q, 119L, 144L, 154S/V, 156G/H/S, 171S, 175A, 177K, 181E/N/P/R/S, 183L, 212E/G/K/L, 224A/Q/R, 226R, 230T/V, 247I/L/R/S, 256E/G/M/S/W, 257D/E/G, 260G, 280L/W, 283C/V, 288L/R/W, 291S, 296E/G/L/Q/W, 306G/T, 307K/R, 310H/L/M/N/Q/R/S/T, 315E, 316I/Y, 317E/F/G/Q/V/W, 326C/L, 330A/D/E/G/K, 331D/G/S, 334E, 335Q, 337A/V, 338G/L/M, 342R/S/T, or 345Q/S; or any combinations thereof, wherein the amino acid positions are relative to the reference sequence corresponding to residues 12 to 346 of SEQ ID NO: 54, 244, 648, 862, 956, 1088, 1416, 1612, 1840, 1866, 1954, 2062, 2674, 2754, or 2786, or relative to the reference sequence corresponding to SEQ ID NO: 54, 244, 648, 862, 956, 1088, 1416, 1612, 1840, 1866, 1954, 2062, 2674, 2754, or 2786.

In some embodiments, the amino acid sequence of the engineered RNA ligase comprises at least a substitution at amino acid position 11, 12, 23, 25, 29, 37, 38, 75, 86, 89, 93, 101, 122, 143, 157, 183, 192, 194, 198, 212, 214, 230, 241, 248, 249, 250, 251, 254, 266, 268, 280, 291, 295, 298, 307, 316, 318, 326, 330, or 331, or any combinations thereof, wherein the amino acid positions are relative to the reference sequence corresponding to residues 12 to 346 of SEQ ID NO: 54, 244, 648, 862, 956, 1088, 1416, 1612, 1840, 1866, 1954, 2062, 2674, 2754, or 2786, or relative to the reference sequence corresponding to SEQ ID NO: 54, 244, 648, 862, 956, 1088, 1416, 1612, 1840, 1866, 1954, 2062, 2674, 2754, or 2786.

In some embodiments, the amino acid sequence of the engineered RNA ligase comprises at least a substitution at amino acid position 11, 12, 23, 25, 29, 37, 38, 75, 93, 101, 122, 143, 157, 192, 194, 198, 241, 248, 249, 250, 251, 254, 268, 295, 298, or 318; a substitution or amino acid residue 86D/L, 89C/D/E/L/N/Q/R/S/T/V/Y, 183L, 212E/G/K/L, 214A/C/H/K/L/Q/Y, 226R, 230T/V, 266C/I, 280L/W, 291H/L/Q/R/S/T/W, 307K/R, 326C/L, 330A/D/E/G/K, or 331D/G/S; or any combinations thereof, wherein the amino acid positions are relative to the reference sequence corresponding to residues 12 to 346 of SEQ ID NO: 54, 244, 648, 862, 956, 1088, 1416, 1612, 1840, 1866, 1954, 2062, 2674, 2754, or 2786, or relative to the reference sequence corresponding to SEQ ID NO: 54, 244, 648, 862, 956, 1088, 1416, 1612, 1840, 1866, 1954, 2062, 2674, 2754, or 2786.

In some embodiments, the amino acid sequence of the engineered RNA ligase further comprises at least a substitution at amino acid position 2, 14, 15, 63, 69, 81, 85, 95, 138, 142, 149, 159, 171, 186, 195, 202, 266, 275, 283, 285, 303, 314, 333, or 339; a substitution or amino acid residue 181, 33L, 39Q/S, 86G, 89G/H/I/M/W, 116N, 117D, 119T, 144W, 154C, 156A/L/N/Q/T, 175K, 177P, 181D, 183V, 185G/K, 212I, 2141, 224Y, 2261, 230D, 247K, 256A, 257L, 260S, 280G/N, 288E, 291P, 296K, 306L, 307E/Q, 310K, 315S/T, 316L, 317A, 326R, 330M/R, 331L/R/W, 334R, 335D/H, 337L, 342I, or 345V; or any combinations thereof, wherein the amino acid positions are relative to the reference sequence corresponding to residues 12 to 346 of SEQ ID NO: 54, 244, 648, 862, 956, 1088, 1416, 1612, 1840, 1866, 1954, 2062, 2674, 2754, or 2786, or relative to the reference sequence corresponding to SEQ ID NO: 54, 244, 648, 862, 956, 1088, 1416, 1612, 1840, 1866, 1954, 2062, 2674, 2754, or 2786.

In some embodiments, the engineered RNA ligase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12 to 346 of SEQ ID NO: 54, or to the reference sequence corresponding to SEQ ID NO: 54, wherein the amino acid sequence comprises one or more substitutions relative to the reference sequence corresponding to residues 12 to 346 of SEQ ID NO: 54, or relative to the reference sequence corresponding to SEQ ID NO: 54.

In some embodiments, the engineered RNA ligase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12 to 346 of an even-numbered SEQ ID NO. of SEQ ID NOs: 220-630, or to the reference sequence corresponding to an even-numbered SEQ ID NO. of SEQ ID NOs: 220-630, wherein the amino acid sequence comprises one or more substitutions relative to the reference sequence corresponding to residues 12 to 346 of SEQ ID NO: 54, or relative to the reference sequence corresponding to SEQ ID NO: 54.

In some embodiments, the amino acid sequence of the engineered RNA ligase comprises at least a substitution or substitution set at amino acid position(s) 156/232/334/335, 12/254/256, 12/230, 12/230/251, 12/230/251/254/256, 12/38/230, 230/251, 12/38/254, 230/256, 254, 230, 12/230/254, 12/38/230/251/256, 12/251, 12/251/254/256, 38, 12/230/256, 12, 12/25/38/230/251, 12/38/102/230/251/256, 12/256, 38/230/256, 256, 12/230/251/256, 230/251/254, 230/254, 251/254, 12/25/230, 38/230, 38/251, 12/38/230/256, 102, 251, 12/25/251/256, 12/102/256, 12/38/230/251/254/256, 12/251/256, 102/230/251/254/256, 12/38/251, 12/230/251/254, 156/248/334/335, 12/25/38, 12/102/230/251/254, 12/38/251/254, 12/251/254, 12/102, 38/230/251/254/256, 12/25/230/251/256, 12/230/311, 12/254, 38/230/251/254, 12/38/251/256, 156/334/335, 257/258/334, 12/25/38/251/256, 334, 12/25/230/254/256, 38/251/256, 12/38/230/251/254, 12/38, 257/258/334/335, 12/38/254/256, 232/248/257/258/334/335, 156/258/334/335, 156/249/334/335, 12/102/230/254, 25, 156/257/258/334/335, 12/102/230, 156/248/257/258/334, 248/249/334/335, 232/257/258/334/335, 334/335, 156/334, 12/38/102/256, 156/257/334/335, 12/140/251/254, 251/254/256, 248/334/335, 156/258/334, 156/248/330/335, 12/38/256, 232/334/335, 156/249/334, 330/334/335, 156/248/334, 156/335, 257/334, 248/334, 232/248/334/335, 12/25/38/230/251/256, 156/248/249/334/335, 330/335, 248/257/334/335, 232/248/334, 156/232/248/334/335, 232/257/330/334, 156/248/291/334/335, 156/232/248/257/258/334, 156/232/249/334/335, 12/25, 156/248/330/334/335, 156/248/249/334, 232/248/330/335, 156/232/330/334/335, 197, 338, 327, 192, 331, 326, 345, 101, 100, 141, 198, 64, 306, 317, 265, 296, 194, 62, 90, 307, 103, 210, 105, or 61, wherein the amino acid positions are relative to the reference sequence corresponding to residues 12 to 346 of SEQ ID NO: 54, or relative to the reference sequence corresponding to SEQ ID NO: 54.

In some embodiments, the engineered RNA ligase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12 to 346 of SEQ ID NO: 244, or to the reference sequence corresponding to SEQ ID NO: 244, wherein the amino acid sequence comprises one or more substitutions relative to the reference sequence corresponding to residues 12 to 346 of SEQ ID NO: 244, or relative to the reference sequence corresponding to SEQ ID NO: 244.

In some embodiments, the engineered RNA ligase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12 to 346 of an even-numbered SEQ ID NO. of SEQ ID NOs: 632-820, or to the reference sequence corresponding to an even-numbered SEQ ID NO. of SEQ ID NOs: 632-820, wherein the amino acid sequence comprises one or more substitutions relative to the reference sequence corresponding to residues 12 to 346 of SEQ ID NO: 244, or relative to the reference sequence corresponding to SEQ ID NO: 244.

In some embodiments, the amino acid sequence of the engineered RNA ligase comprises at least a substitution or substitution set at amino acid position(s) 141/197/326/327, 100/101/338, 141/198/326, 64/100/101, 100/338, 101/141/198/326, 101/198/326/327, 198/326, 64/100/101/338/345, 197/326, 198/327, 141/198/327, 198/326/327, 197/326/327, 64/100/338, 64/100/101/156/197/338, 64/100/101/338, 100/197/338, 197/338, 101/197/326, 64/100/197/338, 101/198, 141/197/327, 64/100/101/197/338, 101/326, 101/197/326/327, 197/327, 197, 141/197, 101, 101/198/326, 64/197/198/338, 141, 101/141, 100/101/197/338, 141/326, 338, 326, 197/198/338, 64/101/338, 64/197/198, 101/197, 100/101, 100/109/197/338, 101/197/338, 197/198, 141/326/327, 100/197, 64/100, 64/197/338, 100/197/198, 101/338, 141/327, 101/306, 64/100/101/197, 101/326/327, 100, 327, 326/345, 64/100/101/197/198, 326/327, 64/101,100/101/197, 101/327, 64/100/197/198/338, 306, 64/338,101/141/326/327, 64/101/197/338, or 101/198/306/327, wherein the amino acid positions are relative to the reference sequence corresponding to residues 12 to 346 of SEQ ID NO: 244, or relative to the reference sequence corresponding to SEQ ID NO: 244.

In some embodiments, the engineered RNA ligase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12 to 346 of SEQ ID NO: 648, or to the reference sequence corresponding to SEQ ID NO: 648, wherein the amino acid sequence comprises one or more substitutions relative to the reference sequence corresponding to residues 12 to 346 of SEQ ID NO: 648, or relative to the reference sequence corresponding to SEQ ID NO: 648.

In some embodiments, the engineered RNA ligase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12 to 346 of an even-numbered SEQ ID NO. of SEQ ID NOs: 822-954, or to the reference sequence corresponding to an even-numbered SEQ ID NO. of SEQ ID NOs: 822-954, wherein the amino acid sequence comprises one or more substitutions relative to the reference sequence corresponding to residues 12 to 346 of SEQ ID NO: 648, or relative to the reference sequence corresponding to SEQ ID NO: 648.

In some embodiments, the amino acid sequence of the engineered RNA ligase comprises at least a substitution or substitution set at amino acid position(s) 194/330/331, 298, 331, 192/193/194/298, 330, 192/330/331, 192/193/194/331, 298/330/331, 194/298/330/331, 192/193/330/331, 192/298/331, 192/193, 194/330, 60/100/156, 193/296/331, 298/331, 193/194/330/331, 194, 194/298/331, 193/298, 193/330, 192, 192/193/194/298/331, 192/193/194/298/330, 192/193/298/331, 100/156, 62/100,298/330, 60/100, 192/193/298/330/331, 100, 193/194/298, 338, 62, 247, 18, 37, 254, 33, 261, 251, 260, 12, 117, 256, 255, 38, 29, 25, or 252, wherein the amino acid positions are relative to the reference sequence corresponding to residues 12 to 346 of SEQ ID NO: 648, or relative to the reference sequence corresponding to SEQ ID NO: 648.

In some embodiments, the engineered RNA ligase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12 to 346 of SEQ ID NO: 862, or to the reference sequence corresponding to SEQ ID NO: 862, wherein the amino acid sequence comprises one or more substitutions relative to the reference sequence corresponding to residues 12 to 346 of SEQ ID NO: 862, or relative to the reference sequence corresponding to SEQ ID NO: 862.

In some embodiments, the engineered RNA ligase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12 to 346 of an even-numbered SEQ ID NO. of SEQ ID NOs: 956-1078, or to the reference sequence corresponding to an even-numbered SEQ ID NO. of SEQ ID NOs: 956-1078, wherein the amino acid sequence comprises one or more substitutions relative to the reference sequence corresponding to residues 12 to 346 of SEQ ID NO: 862, or relative to the reference sequence corresponding to SEQ ID NO: 862.

In some embodiments, the amino acid sequence of the engineered RNA ligase comprises at least a substitution or substitution set at amino acid position(s) 254, 18/254,247/317, 37/317/342, 33, 18/156/254, 18, 156, 254/338, 254/307, 33/37/317, 37, 37/156/317, 156/265/317, 265/317, 156/254, 33/156/317, 317, 156/317, 37/317, 18/338,338, 156/247/317, 33/317, 37/247/265/317, 33/37/156/317, 156/247/296/317, 18/307,156/296/317, 156/247, 37/247/317, 18/254/307, 307, 265, 33/37, 18/156, 33/156/296/317, 18/156/254/338, 37/265/317, 33/37/156/247/317, 296, 33/296/317, 156/296, 247/296/317, 37/296/317, 247/296, 33/37/156, 296/317, 156/254/307, 18/156/307, or 33/37/265, wherein the amino acid positions are relative to the reference sequence corresponding to residues 12 to 346 of SEQ ID NO: 862, or relative to the reference sequence corresponding to SEQ ID NO: 862.

In some embodiments, the engineered RNA ligase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12 to 346 of SEQ ID NO: 956, or to the reference sequence corresponding to SEQ ID NO: 956, wherein the amino acid sequence comprises one or more substitutions relative to the reference sequence corresponding to residues 12 to 346 of SEQ ID NO: 956, or relative to the reference sequence corresponding to SEQ ID NO: 956.

In some embodiments, the engineered RNA ligase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12 to 346 of an even-numbered SEQ ID NO. of SEQ ID NOs: 1080-1374, or to the reference sequence corresponding to an even-numbered SEQ ID NO. of SEQ ID NOs: 1080-1374, wherein the amino acid sequence comprises one or more substitutions relative to the reference sequence corresponding to residues 12 to 346 of SEQ ID NO: 956, or relative to the reference sequence corresponding to SEQ ID NO: 956.

In some embodiments, the amino acid sequence of the engineered RNA ligase comprises at least a substitution or substitution set at amino acid position(s) 12/25/29/156, 12/255/256/257, 12/156/255, 247/252, 12/25, 12, 12/25/29/156/255, 256, 12/156, 12/25/156/255/256/257/261, 18/247/251, 25/255/256/257, 12/25/257, 255/257, 12/25/29/255/257, 256/257, 12/255/257, 12/156/255/256/257, 25, 18/38/251/252, 12/256/257/261, 12/29, 12/25/255/256/257/261, 38, 1225/29/256/257, 29/257, 12/261,251, 249/251, 12/255, 12/29/156/256/257, 252, 255/257/261, 18, 29/255/257, 255/256/257, 12/25/29/156/256/257, 25/255/256/257/261, 29/255/256/257/261, 25/256, 18/38/247/252/327, 18/247, 25/29, 12/25/156/255/257, 18/156/251/252, 12/255/261, 25/255, 12/255/256/261, 156/255/257, 156/257, 12/156/256/257, 25/156/256/257, 12/25/29/156/257/261, 25/261, 12/156/255/256/261, 12/25/29/255/256/257, 38/252,255, 249/252, 12/156/255/256, 12/25/29/261, 257, 12/255/256, 12/29/255, 12/255/257/261, 12/156/257, 192/249/251/252, 12/25/29/255/261, 12/156/255/257, 12/29/255/257, 12/29/256/257, 18/252,249, 156/250, 38/251/252, 156/255/256/257, 25/257, 12/255/256/257/261, 25/156/261, 156/252, 12/25/255/256/257, 12/25/29/156/255/257, 29/156,251/252, 256/257/261, 25/156/255/256/257, 156/249/251/252, 25/29/255, 12/29/156, 255/256, 38/250/252, 247/249/252, 12/256/257, 60, 12/256, 12/156/256, 25/255/257/261, 12/25/29/255/256/261, 12/29/156/255/256, 12/25/156/255/256, 247/251/252, 256/261, 38/251, 12/25/29/156/261, 12/257,156/255/256/261, 12/25/156/255, 12/25/29/156/255/257/261, 12/29/256/261, 29/156/261, 25/29/156/257/261, 12/25/255/256/261, or 12/156/255/261, wherein the amino acid positions are relative to the reference sequence corresponding to residues 12 to 346 of SEQ ID NO: 956, or relative to the reference sequence corresponding to SEQ ID NO: 956.

In some embodiments, the amino acid sequence of the engineered RNA ligase comprises at least a substitution at amino acid position 157, 183, 95, 185, 181, 213, 89, 28, 190, 214, or 143, or any combinations thereof, wherein the amino acid positions are relative to the reference sequence corresponding to residues 12 to 346 of SEQ ID NO: 956, or relative to the reference sequence corresponding to SEQ ID NO: 956.

In some embodiments, the engineered RNA ligase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12 to 346 of SEQ ID NO: 1088, or to the reference sequence corresponding to SEQ ID NO: 1088, wherein the amino acid sequence comprises one or more substitutions relative to the reference sequence corresponding to residues 12 to 346 of SEQ ID NO: 1088, or relative to the reference sequence corresponding to SEQ ID NO: 1088.

In some embodiments, the engineered RNA ligase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12 to 346 of an even-numbered SEQ ID NO. of SEQ ID NOs: 1376-1434, or to the reference sequence corresponding to an even-numbered SEQ ID NO. of SEQ ID NOs: 1376-1434, wherein the amino acid sequence comprises one or more substitutions relative to the reference sequence corresponding to residues 12 to 346 of SEQ ID NO: 1088, or relative to the reference sequence corresponding to SEQ ID NO: 1088.

In some embodiments, the amino acid sequence of the engineered RNA ligase comprises at least a substitution or substitution set at amino acid position(s) 95/157, 95/183/185/214, 89/183, 28/89/143/183, 28/143/157, 181/183, 143/181/183/214, 143/181/183, 89/143/183, 28/143/181/183, 143/157, 95/181/183, 157, 143/183, 89/143/157/183, 89/95/157, 89/157, 89/95/143/181, 95/143, 89/157/183/214, 143/214, 143/181/183/185, 28/95/143, 143/183/214, 28/95/183/214, 89/143/157, 28/95, 95/143/183/185, or 95/143/157/183, wherein the amino acid positions are relative to the reference sequence corresponding to residues 12 to 346 of SEQ ID NO: 1088, or relative to the reference sequence corresponding to SEQ ID NO: 1088.

In some embodiments, the engineered RNA ligase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12 to 346 of SEQ ID NO: 1416, or to the reference sequence corresponding to SEQ ID NO: 1416, wherein the amino acid sequence comprises one or more substitutions relative to the reference sequence corresponding to residues 12 to 346 of SEQ ID NO: 1416, or relative to the reference sequence corresponding to SEQ ID NO: 1416.

In some embodiments, the engineered RNA ligase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12 to 346 of an even-numbered SEQ ID NO. of SEQ ID NOs: 1436-1470, 1478-1766, and 1908-1946, or to the reference sequence corresponding to an even-numbered SEQ ID NO. of SEQ ID NOs: 1436-1470, 1478-1766, and 1908-1946, wherein the amino acid sequence comprises one or more substitutions relative to the reference sequence corresponding to residues 12 to 346 of SEQ ID NO: 1416, or relative to the reference sequence corresponding to SEQ ID NO: 1416.

In some embodiments, the amino acid sequence of the engineered RNA ligase comprises at least a substitution or substitution set at amino acid position(s) 83/137/150, 137/213, 137, 150/190, 143/150, 104/150, 60/143/150/190, 60/150/190/213, 104/137/154, 123/137, 137/150/154/190, 123/143/150, 60/104/143/150/213, 60/95/143/190, 123/137/190, 60/123,104/123, or 60/123/143, wherein the amino acid positions are relative to the reference sequence corresponding to residues 12 to 346 of SEQ ID NO: 1416, or relative to the reference sequence corresponding to SEQ ID NO: 1416.

In some embodiments, the amino acid sequence of the engineered RNA ligase comprises at least a substitution or substitution set at amino acid position(s) 38, 249, 255, 23, 33, 25, 226, 29, 37, 10, 250, 75, 230, 77, 251, 228, 81, 224, 64, 254, 268, 65, 232, 332, 331, 330, 101, 307, 137, 223, 280, 289, 288, 342, 93, 296, 86, 241, 126, 277, 87, 247, 243, 84, 83, 192, 105, 25/29, 25/29/250, 25/29/75/77/250/255, 25/75/230, 25/77/226/255, 25/230,250/255, 29/75/226/230/255, 29/75/77, 29/75/77/226, 29/75/77/230/255, 29/226, 29/226/230, 29/230/255, 33/228, 33/249, 33/37/228, 33/37/38/228, 33/37/38/228/249, 33/38, 75/250/255, 75/226, 75/226/250/255, 75/226/230/250/255, 75/77, 75/77/255, 75/77/250/255, 75/77/226/255, 75/77/226/250/255, 75/77/230, 75/77/230/250, 75/230, 75/230/255, 75/230/250, 226/255, 226/250, 226/230, 226/230/255, 37/228, 37/228/249, 37/38, 37/38/228, 37/38/228/249, 37/38/249, 38/249/291, 10/228, 10/228/249, 10/33/249, 10/33/37, 10/33/37/38/249, 10/33/38/249, 10/33/38, 10/33/249/251, 10/33/37/228, 10/33/37/249, 10/33/37/38/228/249, 10/33/37/38, 10/249, 10/37, 10/37/228, 10/37/228/249, 10/37/249, 10/37/38/228, 10/37/38/228/249, 10/37/38, 10/38/249, 10/38, 10/38/228, 77/255, 77/250, 77/250/255, 77/226, 77/226/255, 77/230, 77/230/255, 230/255, 230/250, 230/250/255, 23/25/226/255, 23/25/226/230, or 23/25/230, wherein the amino acid positions are relative to the reference sequence corresponding to residues 12 to 346 of SEQ ID NO: 1416, or relative to the reference sequence corresponding to SEQ ID NO: 1416.

In some embodiments, the amino acid sequence of the engineered RNA ligase comprises at least a substitution or substitution set at amino acid position(s) 86, 255, 306, 250, 75, 318, 335, 241, 248, 344, 38, 272, 83, 257, 258, or 323, wherein the amino acid positions are relative to the reference sequence corresponding to residues 12 to 346 of SEQ ID NO: 1416, or relative to the reference sequence corresponding to SEQ ID NO: 1416.

In some embodiments, the engineered RNA ligase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12 to 346 of SEQ ID NO: 1612, or to the reference sequence corresponding to SEQ ID NO: 1612, wherein the amino acid sequence comprises one or more substitutions relative to the reference sequence corresponding to residues 12 to 346 of SEQ ID NO: 1612, or relative to the reference sequence corresponding to SEQ ID NO: 1612.

In some embodiments, the engineered RNA ligase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12 to 346 of an even-numbered SEQ ID NO. of SEQ ID NOs: 1768-1840, or to the reference sequence corresponding to an even-numbered SEQ ID NO. of SEQ ID NOs: 768-1840, wherein the amino acid sequence comprises one or more substitutions relative to the reference sequence corresponding to residues 12 to 346 of SEQ ID NO: 1612, or relative to the reference sequence corresponding to SEQ ID NO: 1612.

In some embodiments, the amino acid sequence of the engineered RNA ligase comprises at least a substitution or substitution set at amino acid position(s) 101, 291, 291/307, 331, 33/75, 33/75/249/291/331, 33/38, 33/93/249, 38/75/291/307, 38/75/249/250, 38/75/93/271, 38/93, 10/75, 10/38/75, 10/38/75/291, 10/38/75/249, 10/38/75/93, 64/224,224, 23/101/307, 23/268, 23/37/64/224/254, 23/64/81, 23/224/268, 23/224/254, 23, 23/64/224, 64, 75/249/307, 93, 33/38/75/249, 75/249, 25/38/75/93/291, 33/38/75, 75, or 23/254, wherein the amino acid positions are relative to the reference sequence corresponding to residues 12 to 346 of SEQ ID NO: 1612, or relative to the reference sequence corresponding to SEQ ID NO: 1612.

In some embodiments, the engineered RNA ligase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12 to 346 of SEQ ID NO: 1840, or to the reference sequence corresponding to SEQ ID NO: 1840, wherein the amino acid sequence comprises one or more substitutions relative to the reference sequence corresponding to residues 12 to 346 of SEQ ID NO: 1840, or relative to the reference sequence corresponding to SEQ ID NO: 1840.

In some embodiments, the engineered RNA ligase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12 to 346 of an even-numbered SEQ ID NO. of SEQ ID NOs: 1842-1906, or to the reference sequence corresponding to an even-numbered SEQ ID NO. of SEQ ID NOs: 1842-1906, wherein the amino acid sequence comprises one or more substitutions relative to the reference sequence corresponding to residues 12 to 346 of SEQ ID NO: 1840, or relative to the reference sequence corresponding to SEQ ID NO: 1840.

In some embodiments, the amino acid sequence of the engineered RNA ligase comprises at least a substitution or substitution set at amino acid position(s) 38/65/192, 38/65, 38/65/224, 38/64, 93/307, 38, 38/224, 38/192/224, 65, 192/224, 75/307, 75/250/268/307, 250/268/307/342, 224, 65/192,268, 64/192, 250/268/291/342, 75/93/249/307/342, 250/291, 38/64/192, 38/64/224, 93/249/307, 38/64/86/192/241, 38/342, 248/250/307, 75/77/250/268/307/342, 93/268,192, 38/192, or 64, wherein the amino acid positions are relative to the reference sequence corresponding to residues 12 to 346 of SEQ ID NO: 1840, or relative to the reference sequence corresponding to SEQ ID NO: 1840.

In some embodiments, the engineered RNA ligase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12 to 346 of SEQ ID NO: 1866, or to the reference sequence corresponding to SEQ ID NO: 1866, wherein the amino acid sequence comprises one or more substitutions relative to the reference sequence corresponding to residues 12 to 346 of SEQ ID NO: 1866, or relative to the reference sequence corresponding to SEQ ID NO: 1866.

In some embodiments, the engineered RNA ligase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12 to 346 of an even-numbered SEQ ID NO. of SEQ ID NOs: 1948-2038 and 2130-2620, or to the reference sequence corresponding to an even-numbered SEQ ID NO. of SEQ ID NOs: 1948-2038 and 2130-2620, wherein the amino acid sequence comprises one or more substitutions relative to the reference sequence corresponding to residues 12 to 346 of SEQ ID NO: 1866, or relative to the reference sequence corresponding to SEQ ID NO: 1866.

In some embodiments, the amino acid sequence of the engineered RNA ligase comprises at least a substitution or substitution set at amino acid position(s) 38/248/251, 38/248/250/251/277, 248/250/251, 38/93/192/248/251, 38/192/248/251, 250/251, 38/248/250/251, 248/251/344, 192/248/250/251, 38/248/250, 38/93/250/251/277, 38/255/280/318, 248, 38/86/241/255/280/318/335, 38/255, 38, 318, 38/101/255/280, 38/318/335, 38/255/318, 192/250/251/344, 38/335,318/335, 38/277, 38/93/251, 192/251, 255, 38/64/318, 38/64/241/255/318, 251, 64/101/255/280/318, 38/101/255/280/318/335, 38/255/280, 38/251/277, 64/318, 38/64/255/318, 241/255/280/318, 38/86/101/241/318, 38/241, 38/255/318/335, 38/64/101/318, 38/64/255/280/318, 241/255/318, or 38/241/335, wherein the amino acid positions are relative to the reference sequence corresponding to residues 12 to 346 of SEQ ID NO: 1866, or relative to the reference sequence corresponding to SEQ ID NO: 1866.

In some embodiments, the amino acid sequence of the engineered RNA ligase comprises at least a substitution or substitution set at amino acid position(s) 89, 122, 181, 28, 125, 157, 238, 330, 212, 143, 208, 290, 161, 214, 84, 270, 91, 295, 316, 286, 301, 170, 264, 289, 190, 183, 128, 60, 310, 131, 302, 267, 288, 185, 291, 129, 280, 298, 158/215, 28/122,276, 274, 271, 215, 294, 106, 58, 135, 45, 299, 315, 204, 332, 169, 305, 154, 158, 297, 95, 337, 324, 150, 179, 283, 206, 236, 266, 213, 336, 178, 99, 184, 282, 57, 177, 109, 191, 107, 160, 144, 56, 159, 341, 325, or 91, wherein the amino acid positions are relative to the reference sequence corresponding to residues 12 to 346 of SEQ ID NO: 1866, or relative to the reference sequence corresponding to SEQ ID NO: 1866.

In some embodiments, the engineered RNA ligase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12 to 346 of SEQ ID NO: 1954, or to the reference sequence corresponding to SEQ ID NO: 1954, wherein the amino acid sequence comprises one or more substitutions relative to the reference sequence corresponding to residues 12 to 346 of SEQ ID NO: 1954, or relative to the reference sequence corresponding to SEQ ID NO: 1954.

In some embodiments, the engineered RNA ligase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12 to 346 of an even-numbered SEQ ID NO. of SEQ ID NOs: 2040-2128, or to the reference sequence corresponding to an even-numbered SEQ ID NO. of SEQ ID NOs: 2040-2128, wherein the amino acid sequence comprises one or more substitutions relative to the reference sequence corresponding to residues 12 to 346 of SEQ ID NO: 1954, or relative to the reference sequence corresponding to SEQ ID NO: 1954.

In some embodiments, the amino acid sequence of the engineered RNA ligase comprises at least a substitution or substitution set at amino acid position(s) 93/232/291/306, 93/232/280/306, 93/241/257/306/335, 232/280, 93/291/306, 93/257, 93/306, 93/280,192/318, 93/280/291, 93/241/291/306, 192, 257/291, 241/257/306, 280/335, 93/291/306/335, 93/335, 93/232/335, 64/318, 93/241, 93/232/241/280, 64/192/318/323, 232/280/306, 93/232,291/335, 257/258, 93/232/241/257/258/280/335, 318/323, 93/241/291/335, 83/86/323, 257/258/291, 101/224/318, 83/192/255/296, 171/232/257/258/280, 83/224/255/318, 64/192/250/318, 101/296, 232/241/280, 64/255/318, 86/250/255, 83/86/101, 83/86, 93/241/258/335, 250/296/323, or 257/258/280, wherein the amino acid positions are relative to the reference sequence corresponding to residues 12 to 346 of SEQ ID NO: 1954, or relative to the reference sequence corresponding to SEQ ID NO: 1954.

In some embodiments, the engineered RNA ligase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12 to 346 of SEQ ID NO: 2062, or to the reference sequence corresponding to SEQ ID NO: 2062, wherein the amino acid sequence comprises one or more substitutions relative to the reference sequence corresponding to residues 12 to 346 of SEQ ID NO: 2062, or relative to the reference sequence corresponding to SEQ ID NO: 2062.

In some embodiments, the engineered RNA ligase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12 to 346 of an even-numbered SEQ ID NO. of SEQ ID NOs: 2622-2746, or to the reference sequence corresponding to an even-numbered SEQ ID NO. of SEQ ID NOs: 2622-2746, wherein the amino acid sequence comprises one or more substitutions relative to the reference sequence corresponding to residues 12 to 346 of SEQ ID NO: 2062, or relative to the reference sequence corresponding to SEQ ID NO: 2062.

In some embodiments, the amino acid sequence of the engineered RNA ligase comprises at least a substitution or substitution set at amino acid position(s) 83/84/330, 91/270/290, 83/181,161/290/330, 181/330, 161/270/330, 270/290, 83/84/270/318/330, 64/83/84/161/192/290/318, 91/161/290/318/330, 161/330, 161/214/270/290/330, 192/270/318, 91/161/192/330, 161/270, 181/270/330, 91/181/192/290, 161/290, 83/161/270, 83/192/318, 214/330, 83/161/290/330, 64/83/161/290/330, 83/161/192/214/318, 122/143/212/295, 161/192/270/290, 181/290, 91/161/214/270/290/318, 91/192/214/270, 214/290, 64/83/161/270/318/330, 91/161/181/330, 161/318/330, 91/270/290/330, 64/181/290/318, 181/270/290/318, 64/83/161/192/270/290/318/330, 64/83/161/214/290/318, 122/192/212, 64/83/91/192/214/330, 83/91/161/181/290/318, 91/161/192/214/270/290, 91/161/181/290/330, 83/91/125/161/270, 28/212,122/212, 28/64/83/122/143/192/212/295/318, 122/295, 91/161/192/318, 28/122/143/212, 64/161/290, 64/122/208/318, 64/83/192/290/318, 64/83/161/290, 181/192/318, 83/161/192/318, 214/270, 83/122, or 192/212, 28/122, wherein the amino acid positions are relative to the reference sequence corresponding to residues 12 to 346 of SEQ ID NO: 2062, or relative to the reference sequence corresponding to SEQ ID NO: 2062.

In some embodiments, the engineered RNA ligase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12 to 346 of SEQ ID NO: 2674, or to the reference sequence corresponding to SEQ ID NO: 2674, wherein the amino acid sequence comprises one or more substitutions relative to the reference sequence corresponding to residues 12 to 346 of SEQ ID NO: 2674, or relative to the reference sequence corresponding to SEQ ID NO: 2674.

In some embodiments, the engineered RNA ligase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12 to 346 of an even-numbered SEQ ID NO. of SEQ ID NOs: 2748-2782, 2822-2914, and 2916-3012, or to the reference sequence corresponding to an even-numbered SEQ ID NO. of SEQ ID NOs: 2748-2782, 2822-2914, and 2916-3012, wherein the amino acid sequence comprises one or more substitutions relative to the reference sequence corresponding to residues 12 to 346 of SEQ ID NO: 2674, or relative to the reference sequence corresponding to SEQ ID NO: 2674.

In some embodiments, the amino acid sequence of the engineered RNA ligase comprises at least a substitution or substitution set at amino acid position(s) 28/291/295/298/315, 28/95/291/295/298/315, 143/295/298/315, 291/295/298, 28/143/295/298, 28/291/295/298, 143/185/291/295/298, 28/143/185/291/298/336, 28/298,280/295, 143/298/336, 143/291/295/298, 291/295/298/315/336, 143/295/298, 28/143/295/298/336, 95/143/295/298, 143/280/290, or 143/291/298, wherein the amino acid positions are relative to the reference sequence corresponding to residues 12 to 346 of SEQ ID NO: 2674, or relative to the reference sequence corresponding to SEQ ID NO: 2674.

In some embodiments, the amino acid sequence of the engineered RNA ligase comprises at least a substitution or substitution set at amino acid position(s) 208, 25, 90, 141, 258, 248, 318, 117, 299, 33, 80, 253, 83, 254, 256, 30, 327, 73, 100, 97, 119, 265, 296, 32, 156, 40, 105, 107, 41/114, 41, or 64, wherein the amino acid positions are relative to the reference sequence corresponding to residues 12 to 346 of SEQ ID NO: 2674, or relative to the reference sequence corresponding to SEQ ID NO: 2674.

In some embodiments, the amino acid sequence of the engineered RNA ligase comprises at least a substitution or substitution set at amino acid position(s) 162, 208, 25, 103, 346, 42, 74, 113, 13, 228, 318, 121, 299, 33, 342, 253, 127, 254, 292, 30, 67, 86, 165, 173, 300, 317, 326, 11, 229, 97, 71, 17, 210, 241, 271, 40, 56, or 95, wherein the amino acid positions are relative to the reference sequence corresponding to residues 12 to 346 of SEQ ID NO: 2674, or relative to the reference sequence corresponding to SEQ ID NO: 2674.

In some embodiments, the engineered RNA ligase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12 to 346 of SEQ ID NO: 2754, or to the reference sequence corresponding to SEQ ID NO: 2754, wherein the amino acid sequence comprises one or more substitutions relative to the reference sequence corresponding to residues 12 to 346 of SEQ ID NO: 2754, or relative to the reference sequence corresponding to SEQ ID NO: 2754.

In some embodiments, the engineered RNA ligase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12 to 346 of an even-numbered SEQ ID NO. of SEQ ID NOs: 2784-2820, or to the reference sequence corresponding to an even-numbered SEQ ID NO. of SEQ ID NOs: 2784-2820, wherein the amino acid sequence comprises one or more substitutions relative to the reference sequence corresponding to residues 12 to 346 of SEQ ID NO: 2754, or relative to the reference sequence corresponding to SEQ ID NO: 2754.

In some embodiments, the amino acid sequence of the engineered RNA ligase comprises at least a substitution or substitution set at amino acid position(s) 106/264/280, 280/330, 28/106/264/291, 28/264/280/330, 57/60/135/264/291, 28/135/264/280, 106/264/280/291, 28/106/264/280, 280/299, 28/57/89/106/264/280/291, 264, 185/291, 28/106/299, 28/214/264/299, 264/330, 28/89/214/291, 28/185/271/310, 122/271, or 89/106/135/264/280/330, wherein the amino acid positions are relative to the reference sequence corresponding to residues 12 to 346 of SEQ ID NO: 2754, or relative to the reference sequence corresponding to SEQ ID NO: 2754.

In some embodiments, the engineered RNA ligase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12 to 346 of SEQ ID NO: 2786, or to the reference sequence corresponding to SEQ ID NO: 2786, wherein the amino acid sequence comprises one or more substitutions relative to the reference sequence corresponding to residues 12 to 346 of SEQ ID NO: 2786, or relative to the reference sequence corresponding to SEQ ID NO: 2786.

In some embodiments, the engineered RNA ligase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12 to 346 of an even-numbered SEQ ID NO. of SEQ ID NOs: 3014-3058, or to the reference sequence corresponding to an even-numbered SEQ ID NO. of SEQ ID NOs: 3014-3058, wherein the amino acid sequence comprises one or more substitutions relative to the reference sequence corresponding to residues 12 to 346 of SEQ ID NO: 2786, or relative to the reference sequence corresponding to SEQ ID NO: 2786.

In some embodiments, the amino acid sequence of the engineered RNA ligase comprises at least a substitution or substitution set at amino acid position(s) 25/228/248/264, 32/113/228/248, 25/32/113/241/248/264/299, 25/113/248/264/318, 25/127/228/264/299/318, 25/113/228/248/327, 228/241/264, 264/299/318/327, 25/113/241/248/264/318, 25/127/248/299/318, 113/127/228/264/299, 25/127, 25/228/248/264/327, 25/113/264/299, 32/113/264/318, 25/32/241/264/327, 113/264/327, 25/127/228/248/318, 327, 32/113/254/264, 25/228/241/264, 25/113/228/241/318, or 113/327, wherein the amino acid positions are relative to the reference sequence corresponding to residues 12 to 346 of SEQ ID NO: 2786, or relative to the reference sequence corresponding to SEQ ID NO: 2786.

In some embodiments, the amino acid sequence of the engineered RNA ligase comprises at least one substitution provided in Tables 6.1, 6.2, 7.1, 8.1, 8.2, 9.1, 10.1, 11.1, 12.1, 13.1, 14.1, 15.1, 16.1, 17.1, 18.1, 19.1, 20.1, 21.1, 22.1, 23.1, 24.1, 25.1, and 26.1, wherein the amino acid positions are relative to the reference sequence corresponding to residues 12 to 346 of SEQ ID NO: 54, 244, 648, 862,956, 1088, 1416, 1612, 1840, 1866, 1954, 2062, 2674, 2754, or 2786, or relative to the reference sequence corresponding to SEQ ID NO: 54, 244, 648, 862, 956, 1088, 1416, 1612, 1840, 1866, 1954, 2062, 2674, 2754, or 2786.

In some embodiments, the amino acid sequence of the engineered RNA ligase comprises at least a substitution or substitution set of an RNA ligase variant provided in Tables 6.1, 6.2, 7.1, 8.1, 8.2, 9.1, 10.1, 11.1, 12.1, 13.1, 14.1, 15.1, 16.1, 17.1, 18.1, 19.1, 20.1, 21.1, 22.1, 23.1, 24.1, 25.1, and 26.1, wherein the amino acid positions are relative to the reference sequence corresponding to residues 12 to 346 of SEQ ID NO: 54, 244, 648, 862, 956, 1088, 1416, 1612, 1840, 1866, 1954, 2062, 2674, 2754, or 2786, or relative to the reference sequence corresponding to SEQ ID NO: 54, 244, 648, 862, 956, 1088, 1416, 1612, 1840, 1866, 1954, 2062, 2674, 2754, or 2786.

In some embodiments, the engineered RNA ligase comprises an amino acid sequence having least 70%, 75%, 80%, 81%, 82%, 83%, 84%, 85%, 86%, 87%, 88%, 89%, 90%, 91%, 92%, 93%, 94%, 95%, 96%, 97%, 98%, 99% or more sequence identity to a reference sequence comprising a substitution or substitution set provided in Tables 6.1, 6.2, 7.1, 8.1, 8.2, 9.1, 10.1, 11.1, 12.1, 13.1, 14.1, 15.1, 16.1, 17.1, 18.1, 19.1, 20.1, 21.1, 22.1, 23.1, 24.1, 25.1, and 26.1, wherein the amino acid positions are relative to the reference sequence corresponding to residues 12 to 346 of SEQ ID NO: 54, 244, 648, 862, 956, 1088, 1416, 1612, 1840, 1866, 1954, 2062, 2674, 2754, or 2786, or relative to the reference sequence corresponding to SEQ ID NO: 54, 244, 648, 862, 956, 1088, 1416, 1612, 1840, 1866, 1954, 2062, 2674, 2754, or 2786.

In some embodiments, the engineered RNA ligase comprises an amino acid sequence comprising residues 12 to 346 of an even numbered SEQ ID NO. of SEQ ID NOs: 4-1470 and 1478-3058, or an amino acid sequence comprising an even numbered SEQ ID NO. of SEQ ID NOs: 4-1470 and 1478-3058. In some embodiments, the amino acid sequence of the engineered RNA ligase optionally has 1, 2, 3, 4, 5, 6, 7, 8, 9, or up to 10 substitutions.

In some embodiments, the engineered RNA ligase comprises an amino acid sequence comprising residues 12 to 346 of SEQ ID NO: 54, 244, 648, 862, 956, 1088, 1416, 1612, 1840, 1866, 1954, 2062, 2674, 2754, or 2786, or an amino acid sequence comprising SEQ ID NO: 54, 244, 648, 862, 956, 1088, 1416, 1612, 1840, 1866, 1954, 2062, 2674, 2754, or 2786. In some embodiments, the amino acid sequence of the engineered RNA ligase optionally has 1, 2, 3, 4, 5, 6, 7, 8, 9, or up to 10 substitutions.

In some embodiments, the engineered RNA ligase has RNA ligase 2 activity. In some embodiments, the engineered RNA ligase has RNA ligase 2 activity and at least one improved property as compared to a reference RNA ligase.

In some embodiments, the improved property of the engineered RNA ligase is selected from i) increased activity, ii) increased stability, iii) increased thermostability, iv) increased product yield, v) increased activity on polynucleotides ligase substrates with phosphorothioate internucleoside linkages, vi) increased activity on oligonucleotides with 2′-modifications, particularly at the ligation junction, vii) increased substrate tolerance, or any combination of i), ii), iii), iv), v), vi), and vii), compared to a reference RNA ligase. In some embodiments, the improved property of the engineered RNA ligase is in comparison to the reference RNA ligase having the sequence corresponding to residues 12 to 346 of SEQ ID NO: 2, 54, 244, 648, 862, 956, 1088, 1416, 1612, 1840, 1866, 1954, 2062, 2674, 2754, or 2786, or the sequence corresponding to SEQ ID NO: 2, 54, 244, 648, 862, 956, 1088, 1416, 1612, 1840, 1866, 1954, 2062, 2674, 2754, or 2786.

In some embodiments, the engineered RNA ligase is purified. In some further embodiments, the engineered RNA ligase is provided in solution, as a lyophilizate, or immobilized on a substrate, such as solid substrates, porous substrates, membranes or particles.

In another aspect, the present disclosure provides a recombinant polynucleotide comprising a polynucleotide sequence encoding the engineered RNA ligases disclosed herein.

In some embodiments, the recombinant polynucleotide comprises a polynucleotide sequence having at least 70%, 75%, 75%, 70%, 75%, 80%, 81%, 82%, 83%, 84%, 85%, 90%, 91%, 92%, 93%, 94%, 95%, 96%, 97%, 98%, 99%, or more sequence identity to a reference polynucleotide sequence corresponding to nucleotide residues 34 to 1038 of SEQ ID NO: 53, 243, 647, 861, 955, 1087, 1415, 1611, 1839, 1865, 1953, 2061, 2673, 2753, or 2785, or to a reference polynucleotide sequence corresponding to SEQ ID NO: 53, 243, 647, 861, 955, 1087, 1415, 1611, 1839, 1865, 1953, 2061, 2673, 2753, or 2785, wherein the recombinant polynucleotide encodes an RNA ligase.

In some embodiments, the recombinant polynucleotide comprises a polynucleotide sequence having at least 70%, 75%, 70%, 75%, 80%, 81%, 82%, 83%, 84%, 85%, 90%, 91%, 92%, 93%, 94%, 95%, 96%, 97%, 98%, 99%, or more sequence identity to a reference polynucleotide sequence corresponding to nucleotide residues 34 to 1038 of an odd numbered SEQ ID NO. of SEQ ID NOs: 3-1469 and 1477-3057, or to a reference polynucleotide sequence corresponding an odd numbered SEQ ID NO. of SEQ ID NOs: 3-1469 and 1477-3057, wherein the recombinant polynucleotide encodes an RNA ligase.

In some embodiments, the polynucleotide sequence of the recombinant polynucleotide encoding an engineered RNA ligase is codon optimized for expression in an organism or cell type thereof, for example a bacterial cell, a fungal cell, insect cell, or mammalian cell.

In some embodiments, the recombinant polynucleotide comprises a polynucleotide sequence comprising nucleotide residues 34 to 1038 of SEQ ID NO. 53, 243, 647, 861, 955, 1087, 1415, 1611, 1839, 1865, 1953, 2061, 2673, 2753, or 2785, or a polynucleotide sequence comprising SEQ ID NOs: 53, 243, 647, 861, 955, 1087, 1415, 1611, 1839, 1865, 1953, 2061, 2673, 2753, or 2785.

In some embodiments, the recombinant polynucleotide comprises a polynucleotide sequence comprising nucleotide residues 34 to 1038 of an odd numbered SEQ ID NO. of SEQ ID NOs: 3-1469 and 1477-3057, or a polynucleotide sequence comprising an odd numbered SEQ ID NO. of SEQ ID NOs: 3-1469 and 1477-3057.

In a further aspect, the present disclosure provides expression vectors comprising at least one recombinant polynucleotide provided herein encoding an engineered RNA ligase. In some embodiments, the recombinant polynucleotide of the expression vector is operably linked to a control sequence. In some embodiments, the control sequence comprises a promoter, particularly a heterologous promoter.

In another aspect, the present disclosure also provides a host cell comprising at least one expression vector provided herein. In some embodiments, the host cell is a prokaryotic cell or a eukaryotic cell. In some embodiments, the host cell is a bacterial cell, fungal cell, insect cell, or mammalian cell. In some preferred embodiments, the host cell is a bacterial cell, such as E. coli. or B. subtilis.

In a further aspect, the present disclosure provides a method of producing an engineered RNA ligase polypeptide, the method comprising culturing a host cell described herein under suitable culture conditions such that at least one engineered RNA ligase is produced. In some embodiments, the method further comprises recovering or isolating the engineered RNA ligase from the culture and/or host cells. In some embodiments, the method further comprises the step of purifying the engineered RNA ligase.

In another aspect, the present disclosure provides a composition comprising at least one engineered RNA ligase disclosed herein. In some embodiments, the composition comprises at least a buffer. In some embodiments, the composition further comprises a nucleotide substrate (e.g., ATP or dATP) and one or more of polynucleotide substrates, such as synthetic polynucleotides, particularly modified polynucleotides.

In a further aspect, the present disclosure provides a method of ligating at least a first polynucleotide strand and a second polynucleotide strand, comprising contacting the first polynucleotide strand and a second polynucleotide strand with an engineered RNA ligase described herein in presence of a nucleotide substrate under conditions suitable for ligation of the first polynucleotide strand to the second polynucleotide strand, wherein the first polynucleotide strand comprises a ligatable 5′-end and the second polynucleotide strand comprises a 3′-end ligatable to the 5-end of the first polynucleotide strand. In some embodiments, the first polynucleotide strand and/or second polynucleotide strand comprises RNA or a mixture of RNA and DNA.

In some embodiments, the method further comprises a third polynucleotide strand, wherein the first polynucleotide strand and second polynucleotide strand hybridize adjacent to one another on the third polynucleotide to position the 5′-end of the first polynucleotide strand adjacent to the 3′-end of the second polynucleotide strand to form a nick. In some embodiments, the third polynucleotide strand is continuous with the first polynucleotide strand or second polynucleotide strand. In some embodiments, the third polynucleotide strand is continuous with the first polynucleotide strand and second polynucleotide strand to form a single continuous polynucleotide substrate.

In some embodiments, 3′-end region of the second polynucleotide strand that hybridizes to the third polynucleotide strand is at least 4, 6, 8 or more base pairs in length. In some embodiments, the 5-end region of the first polynucleotide strand that hybridizes to the third polynucleotide strand is at least 4, 6, 8 or more base pairs in length.

In some embodiments of the method, the third polynucleotide strand comprises a splint or bridging polynucleotide, wherein the 5′-terminal sequence of the first polynucleotide strand and the 3′-terminal sequence of the second polynucleotide strand hybridize adjacent to one another on the splint or bridging polynucleotide to position the 5′-end of the first polynucleotide strand adjacent to the 3′-end of the second polynucleotide strand.

In some embodiments, the first and third polynucleotide strands hybridize to each other to form a first double stranded polynucleotide fragment, and the second polynucleotide strand hybridizes to another polynucleotide strand to form a second double stranded fragment, wherein the first and second double stranded fragments have complementary ends that can base pair to form substrates for the engineered RNA ligase.

In some embodiments, the engineered RNA ligase is used in a method to synthesize RNA or DNA/RNA polynucleotides by ligation of shorter RNA or DNA/RNA oligonucleotides. In some embodiments, the engineered RNA ligase is used to repair nicks in dsRNA or dsRNA/DNA. In some embodiments, the engineered RNA ligase is used to ligate the 3′ OH of RNA to the 5′ phosphate of DNA or RNA. In some embodiments, the engineered RNA ligase is used to ligate the 3′ OH of RNA to the 5′ phosphate of DNA in a double-stranded-format NGS RNA library construction. In some embodiments, the engineered RNA ligase is used in a method of preparing RNA rings.

In a further aspect, the present disclosure also provides a kit comprising at least one engineered RNA ligase disclosed herein. In some embodiments, the kit further comprises one or more of a buffer, nucleotide substrate (e.g., ATP or dATP), ligation enhancing agent, and/or one or more polynucleotide substrates.

### BRIEF DESCRIPTION OF THE DRAWINGS

FIG. 1 shows the output of a Gaussian Process Regression (GPR) analysis for two recombinant single stranded RNA ligase for a RNA ligase substrate for ligase reaction variables of MgCl₂, ATP, and oligonucleotide substrate concentration.

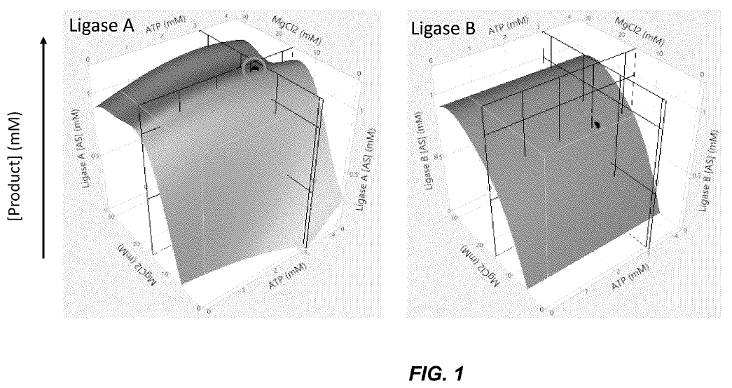

### DETAILED DESCRIPTION

The present disclosure provides engineered RNA ligase polypeptides and compositions thereof, as well as polynucleotides encoding the engineered RNA ligase polypeptides. The disclosure also provides methods of using of the engineered RNA ligase polypeptides and compositions thereof for molecular biological, diagnostic, and other purposes. In some embodiments, the engineered RNA ligase polypeptides display, among others, increased activity, increased stability, increased product yield, and/or increased activity on polynucleotides containing one or more modified nucleotides or nucleotide analogs.

### Abbreviations and Definitions

In reference to the present disclosure, the technical and scientific terms used in the descriptions herein will have the meanings commonly understood by one of ordinary skill in the art, unless specifically defined otherwise. Accordingly, the following terms are intended to have the following meanings.

As used herein, the singular forms “a”, “an” and “the” include plural referents unless the context clearly indicates otherwise. Thus, for example, reference to “a polypeptide” includes more than one polypeptide.

Similarly, “comprise,” “comprises,” “comprising” “include,” “includes,” and “including” are interchangeable and not intended to be limiting. Thus, as used herein, the term “comprising” and its cognates are used in their inclusive sense (i.e., equivalent to the term “including” and its corresponding cognates).

It is to be further understood that where descriptions of various embodiments use the term “comprising,” those skilled in the art would understand that in some specific instances, an embodiment can be alternatively described using language “consisting essentially of” or “consisting of.”

“About” means an acceptable error for a particular value. In some instances, “about” means within 0.05%, 0.5%, 1.0%, or 2.0%, of a given value range. In some instances, “about” means within 1, 2, 3, or 4 standard deviations of a given value.

“EC” number refers to the Enzyme Nomenclature of the Nomenclature Committee of the International Union of Biochemistry and Molecular Biology (NC-IUBMB). The IUBMB biochemical classification is a numerical classification system for enzymes based on the chemical reactions they catalyze.

“ATCC” refers to the American Type Culture Collection whose biorepository collection includes genes and strains.

“NCBI” refers to National Center for Biological Information and the sequence databases provided therein.

“Protein,” “polypeptide,” and “peptide” are used interchangeably to denote a polymer of at least two amino acids covalently linked by an amide bond, regardless of length or post-translational modification (e.g., glycosylation or phosphorylation).

“Amino acids” and “amino acid” are referred to herein by either their commonly known three-letter symbols or by the one-letter symbols recommended by IUPAC-IUB Biochemical Nomenclature Commission. The abbreviations used for the genetically encoded amino acids are conventional and are as follows: alanine (Ala or A), arginine (Arg or R), asparagine (Asn or N), aspartate (Asp or D), cysteine (Cys or C), glutamate (Glu or E), glycine (Gly or G), glutamine (Gln or Q), histidine (His or H), isoleucine (Ile or I), leucine (Leu or L), lysine (Lys or K), methionine (Met or M), phenylalanine (Phe or F), proline (Pro or P), serine (Ser or S), threonine (Thr or T), tryptophan (Trp or W), tyrosine (Tyr or Y), and valine (Val or V). When the three-letter abbreviations are used, unless specifically preceded by an “L” or a “D” or clear from the context in which the abbreviation is used, the amino acid may be in either the L- or D-configuration about «-carbon (Ca). For example, whereas “Ala” designates alanine without specifying the configuration about the «-carbon, “D-Ala” and “L-Ala” designate D-alanine and L-alanine, respectively. When the one-letter abbreviations are used, upper case letters designate amino acids in the L-configuration about the «-carbon and lower case letters designate amino acids in the D-configuration about the «-carbon. For example, “A” designates L-alanine and “a” designates D-alanine. When polypeptide sequences are presented as a string of one-letter or three-letter abbreviations (or mixtures thereof), the sequences are presented in the amino (N) to carboxy (C) direction in accordance with common convention.

“Fusion protein,” and “chimeric protein” and “chimera” refer to hybrid proteins created through the joining of two or more polynucleotides that originally encode separate proteins. In some embodiments, fusion proteins are created by recombinant technology (e.g., molecular biology techniques known in the art).

“RNA ligase” refers to enzymes that covalently joins the 5′-phosphoryl termini of RNA or DNA to the 3′-hydroxyl termini of RNA or DNA. Two families of RNA ligases are known to occur in nature. RNA ligase 1 catalyzes the covalent joining of single stranded 5′-phosphoryl termini of RNA or DNA to single stranded 3-hydroxyl termini of RNA or DNA. RNA ligase 2 also catalyzes the covalent joining of a 3′-hydroxyl terminus of RNA to a 5′-phosphorylated RNA or DNA but shows preference for double stranded substrates. In some embodiments, RNA ligases include those enzymes classified in EC 6.5.1.3.

Without being bound by any theory of operation, although some DNA ligases are capable of acting on either DNA or RNA as the 3′-hydroxyl strand substrate, an RNA ligase 2 acting at a duplex nick preferentially acts on 3′-hydroxyl strand of RNA but is agnostic to whether 5-phosphoryl strand is DNA or RNA. Moreover, in some embodiments, the strand with the 3′-hydroxyl may include deoxyribonucleotides if sufficient number of ribonucleotides are present at the 3′-hydroxyl terminus. For example, the RNA specificity of T4 RNA ligase 2 is affected by the 3′-hydroxyl strand, and specifically by the two terminal ribonucleotides of the 3-′hydroxyl side of the nick (see, e.g., Nandakumar et al., Mol. Cell., 2004, 16:211-221). It is to be understood that the ligation reaction is not limited to naturally occurring RNA and DNA substrates also includes polynucleotide substrates that contain modified nucleotides and/or nucleotide analogs.

“Polynucleotide,” “nucleic acid,” or “oligonucleotide” is used herein to denote a polymer comprising at least two nucleotides where the nucleotides are either deoxyribonucleotides or ribonucleotides or mixtures of deoxyribonucleotides and ribonucleotides. In some embodiments, the abbreviations used for genetically encoding nucleosides are conventional and are as follow: adenosine (A); guanosine (G); cytidine (C); thymidine (T); and uridine (U). Unless specifically delineated, the abbreviated nucleosides may be either ribonucleosides or 2′-deoxyribonucleosides. The nucleosides may be specified as being either ribonucleosides or 2′-deoxyribonucleosides on an individual basis or on an aggregate basis. When a polynucleotide, nucleic acid, or oligonucleotide sequences are presented as a string of one-letter abbreviations, the sequences are presented in the 5′ to 3′ direction in accordance with common convention, and the phosphates are not indicated. The term “DNA” refers to deoxyribonucleic acid. The term “RNA” refers to ribonucleic acid. The polynucleotide or nucleic acid may be single-stranded or double-stranded, or may include both single-stranded regions and double-stranded regions.

In some embodiments, the terms “polynucleotide,” “nucleic acid” and “oligonucleotide” encompass polynucleotide or nucleic acid or oligonucleotide analogs or modified polynucleotide or nucleic acid or oligonucleotide, which include, among others, nucleosides linked together via other than standard phosphodiester linkages, such as non-standard linkages of phosphoramidates, phosphorothioates, amide linkages, etc.; nucleosides with modified and/or synthetic nucleobases, for example inosine, xanthine, hypoxanthine, etc.; nucleosides with modified sugar residues, such as 2′-O-alkyl, 2′-halo, 2,3-dideoxy, 2′-halo-2′-deoxy, β-D-ribo LNA, α-L-ribo-LNA (e.g., locked nucleic acids), etc.; and/or 5′-phosphate analogs, including, among others, phosphorothioate, phosphoacetate, phosphoramidate, monomethylphosphate, methylphosphonate, or phosphonocarboxylate.

“Duplex” and “ds” refer to a double-stranded nucleic acid (e.g., DNA or RNA) molecule comprised of two single-stranded polynucleotides that are complementary in their sequence (e.g., A pairs to T or U, C pairs to G), arranged in an antiparallel 5′ to 3′ orientation, and held together by hydrogen bonds between the nucleobases (e.g., adenine [A], guanine [G], cytosine [C], thymine [T], uridine [U]).

“Complementary” is used herein to describe the structural relationship between nucleotide bases that are capable of forming base pairs with one another. For example, a purine nucleotide base present on a polynucleotide that is complementary to a pyrimidine nucleotide base on a polynucleotide may base pair by forming hydrogen bonds with one another. Complementary nucleotide bases can base pair via Watson/Crick base pairing or in any other manner than forms stable duplexes or other nucleic acid structures.

“Watson/Crick Base-Pairing” refers to a pattern of specific pairs of nucleobases and analogs that bind together through sequence-specific hydrogen-bonds, e.g. A pairs with T or U, and G pairs with C.

“Annealing” or “Hybridization” refers to the base-pairing interactions of one nucleobase polymer (e.g., poly- and oligonucleotides) with another that results in the formation of a double-stranded structure, a triplex structure or a quaternary structure. Annealing or hybridization can occur via Watson-Crick base-pairing interactions, but may be mediated by other hydrogen-bonding interactions, such as Hoogsteen base pairing. In some embodiments, the nucleobase polymer that anneals or hybridizes to another is a single nucleobase polymer while in other embodiments, the nucleobase polymers are separate nucleobase polymers.

“Engineered,” “recombinant,” “non-naturally occurring,” and “variant,” when used with reference to a cell, a polynucleotide or a polypeptide refer to a material or a material corresponding to the natural or native form of the material that has been modified in a manner that would not otherwise exist in nature or is identical thereto but produced or derived from synthetic materials and/or by manipulation using recombinant techniques.

“Wild-type” and “naturally-occurring” refer to the form found in nature. For example, a wild-type polypeptide or polynucleotide sequence is a sequence present in an organism that can be isolated from a source in nature and which has not been intentionally modified by human manipulation.

“Coding sequence” and synonymously “encoding” refers to that part of a nucleic acid (e.g., a gene) that encodes an amino acid sequence of a protein.

“Percent (%) sequence identity” refers to comparisons among polynucleotides and polypeptides, and are determined by comparing two optimally aligned sequences over a comparison window, wherein the portion of the polynucleotide or polypeptide sequence in the comparison window may comprise additions or deletions (i.e., gaps) as compared to the reference sequence for optimal alignment of the two sequences. The percentage may be calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity. Alternatively, the percentage may be calculated by determining the number of positions at which either the identical nucleic acid base or amino acid residue occurs in both sequences or a nucleic acid base or amino acid residue is aligned with a gap to yield the number of matched positions, dividing the number of matched positions by the total number of positions in the window of comparison and multiplying the result by 100 to yield the percentage of sequence identity. Those of skill in the art appreciate that there are many established algorithms available to align two sequences. Optimal alignment of sequences for comparison can be conducted, e.g., by the local homology algorithm of Smith and Waterman (Smith and Waterman, Adv. Appl. Math., 1981, 2:482), by the homology alignment algorithm of Needleman and Wunsch (Needleman and Wunsch, J. Mol. Biol., 1970, 48:443), by the search for similarity method of Pearson and Lipman (Pearson and Lipman, Proc. Natl. Acad. Sci. USA, 1988, 85:2444), by computerized implementations of these algorithms (e.g., GAP, BESTFIT, FASTA, and TFASTA in the GCG Wisconsin Software Package), or by visual inspection, as known in the art. Examples of algorithms that are suitable for determining percent sequence identity and sequence similarity include, but are not limited to the BLAST and BLAST 2.0 algorithms (see, e.g., Altschul et al., J. Mol. Biol., 1990, 215:403-410; and Altschul et al., Nucleic Acids Res., 1977, 3389-3402). Software for performing BLAST analyses is publicly available through the National Center for Biotechnology Information website. This algorithm involves first identifying high scoring sequence pairs (HSPs) by identifying short words of length “W” in the query sequence, which either match or satisfy some positive-valued threshold score “T,” when aligned with a word of the same length in a database sequence. T is referred to as the neighborhood word score threshold (see Altschul et al, supra). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters “M” (reward score for a pair of matching residues; always >0) and “N” (penalty score for mismatching residues; always <0). For amino acid sequences, a scoring matrix is used to calculate the cumulative score. Extension of the word hits in each direction are halted when: the cumulative alignment score falls off by the quantity “X” from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, M=5, N=−4, and a comparison of both strands. For amino acid sequences, the BLASTP program uses as defaults a wordlength (W) of 3, an expectation (E) of 10, and the BLOSUM62 scoring matrix (see, e.g., Henikoff and Henikoff, Proc. Natl. Acad. Sci. USA, 1989, 89:10915). Exemplary determination of sequence alignment and % sequence identity can employ the BESTFIT or GAP programs in the GCG Wisconsin Software package (Accelrys, Madison WI), using default parameters provided.

“Reference sequence” refers to a defined sequence used as a basis for a sequence comparison. A reference sequence may be a subset of a larger sequence, for example, a segment of a full-length gene or polypeptide sequence. Generally, a reference sequence is at least 20 nucleotide or amino acid residues in length, at least 25 residues in length, at least 50 residues in length, at least 100 residues in length or the full length of the nucleic acid or polypeptide. Since two polynucleotides or polypeptides may each (1) comprise a sequence (i.e., a portion of the complete sequence) that is similar between the two sequences, and (2) may further comprise a sequence that is divergent between the two sequences, sequence comparisons between two (or more) polynucleotides or polypeptide are typically performed by comparing sequences of the two polynucleotides or polypeptides over a “comparison window” to identify and compare local regions of sequence similarity. In some embodiments, a “reference sequence” can be based on a primary amino acid sequence, where the reference sequence is a sequence that can have one or more changes in the primary sequence. For instance, the phrase “a reference sequence corresponding to SEQ ID NO: 2, having an asparagine at the residue corresponding to X11” (or “a reference sequence corresponding to SEQ ID NO: 2, having an asparagine at the residue corresponding to position 11”) refers to a reference sequence in which the corresponding residue at position X11 in SEQ ID NO: 2 (e.g., an glycine), has been changed to asparagine.

“Comparison window” refers to a conceptual segment of contiguous nucleotide positions or amino acids residues wherein a sequence may be compared to a reference sequence. In some embodiments, the comparison window is at least 15 to 20 contiguous nucleotides or amino acids and wherein the portion of the sequence in the comparison window may comprise additions or deletions (i.e., gaps) of 20 percent or less as compared to the reference sequence (which does not comprise additions or deletions) for optimal alignment of the two sequences. In some embodiments, the comparison window can be longer than 15-20 contiguous residues, and includes, optionally 30, 40, 50, 100, or longer windows.

“Corresponding to”, “reference to,” and “relative to” when used in the context of the numbering of a given amino acid or polynucleotide sequence refer to the numbering of the residues of a specified reference sequence when the given amino acid or polynucleotide sequence is compared to the reference sequence. In other words, the residue number or residue position of a given polymer is designated with respect to the reference sequence rather than by the actual numerical position of the residue within the given amino acid or polynucleotide sequence. For example, a given amino acid sequence, such as that of an engineered RNA ligase, can be aligned to a reference sequence by introducing gaps to optimize residue matches between the two sequences. In these cases, although the gaps are present, the numbering of the residue in the given amino acid or polynucleotide sequence is made with respect to the reference sequence to which it has been aligned. In some embodiments, the sequence is tagged (e.g., with a histidine tag).

“Mutation” refers to the alteration of a nucleic acid sequence. In some embodiments, mutations result in changes to the encoded polypeptide sequence (i.e., as compared to the original sequence without the mutation). In some embodiments, the mutation comprises a substitution, such that a different amino acid is produced. In some alternative embodiments, the mutation comprises an addition, such that an amino acid is added (e.g., insertion) to the original polypeptide sequence. In some further embodiments, the mutation comprises a deletion, such that an amino acid is deleted from the original polypeptide sequence. Any number of mutations may be present in a given sequence.

“Amino acid difference” and “residue difference” refer to a difference in the amino acid residue at a position of a polypeptide sequence relative to the amino acid residue at a corresponding position in a reference sequence. The amino acid positions of amino acid differences generally are referred to herein as “Xn,” where n refers to the corresponding position in the reference sequence upon which the residue difference is based. For example, a “residue difference at position X11 as compared to SEQ ID NO: 2” (or a “residue difference at position 11 as compared to SEQ ID NO: 2”) refers to a difference of the amino acid residue at the polypeptide position corresponding to position 11 of SEQ ID NO: 2. Thus, if the reference polypeptide of SEQ ID NO: 2 has a glycine at position 11, then a “residue difference at position X11 as compared to SEQ ID NO: 2” refers to an amino acid substitution of any residue other than glycine at the position of the polypeptide corresponding to position 11 of SEQ ID NO: 2. In some instances herein, the specific amino acid residue difference at a position is indicated as “XnY” where “Xn” specified the corresponding residue and position of the reference polypeptide (as described above), and “Y” is the single letter identifier of the amino acid found in the engineered polypeptide (i.e., the different residue than in the reference polypeptide). In some instances (e.g., in the Tables in the Examples), the present disclosure also provides specific amino acid differences denoted by the conventional notation “AnB”, where A is the single letter identifier of the residue in the reference sequence, “n” is the number of the residue position in the reference sequence, and B is the single letter identifier of the residue substitution in the sequence of the engineered polypeptide. In some embodiments, the amino acid difference, e.g., a substitution, is denoted by the abbreviation “nB,” without the identifier for the residue in the reference sequence. In some instances, an amino acid residue difference or substitution may be a deletion and may be denoted by a “-” where appropriate. In some embodiments, the phrase “an amino acid residue nB” denotes the presence of the amino acid residue in the engineered polypeptide, which may or may not be a substitution in context of a reference polypeptide or amino acid sequence.

In some instances, a polypeptide of the present disclosure can include one or more amino acid residue differences relative to a reference sequence, which is indicated by a list of the specified positions where residue differences are present relative to the reference sequence. In some embodiments, where more than one amino acid can be used in a specific residue position of a polypeptide, the various amino acid residues that can be used are separated by a “/” (e.g., X18A/X18H, X18A/H, or 18A/H). The present disclosure includes engineered polypeptide sequences comprising one or more amino acid differences that include either/or both conservative and non-conservative amino acid substitutions, as well as insertions and deletions of amino acids in the sequence.

“Amino acid substitution set” and “substitution set” refers to a group of amino acid substitutions within a polypeptide sequence. In some embodiments, substitution sets comprise 2, 3, 4, 5, 6, 7, 8, 9, 10, 11, 12, 13, 14, 15, or more amino acid substitutions. In some embodiments, a substitution set refers to the set of amino acid substitutions that is present in any of the variant RNA ligase polypeptides listed in any of the Tables in the Examples. In some embodiments, the amino acid sequence comprises at least each of the amino acid substitutions in the referenced substitution set. In the substitution sets, the individual substitutions are separated by a semicolon (“;”; e.g., S106G; K251F) or slash (“/”; e.g., S106G/K251F or 106G/251F).

“Conservative amino acid substitution” refers to a substitution of a residue with a different residue having a similar side chain, and thus typically involves substitution of the amino acid in the polypeptide with amino acids within the same or similar defined class of amino acids. By way of example and not limitation, an amino acid with an aliphatic side chain may be substituted with another aliphatic amino acid (e.g., alanine, valine, leucine, and isoleucine); an amino acid with hydroxyl side chain is substituted with another amino acid with a hydroxyl side chain (e.g., serine and threonine); an amino acids having aromatic side chains is substituted with another amino acid having an aromatic side chain (e.g., phenylalanine, tyrosine, tryptophan, and histidine); an amino acid with a basic side chain is substituted with another amino acid with a basis side chain (e.g., lysine and arginine); an amino acid with an acidic side chain is substituted with another amino acid with an acidic side chain (e.g., aspartic acid or glutamic acid); and a hydrophobic or hydrophilic amino acid is replaced with another hydrophobic or hydrophilic amino acid, respectively.

“Non-conservative substitution” refers to substitution of an amino acid in the polypeptide with an amino acid with significantly differing side chain properties. Non-conservative substitutions may use amino acids between, rather than within, the defined groups and affect: (a) the structure of the peptide backbone in the area of the substitution (e.g., proline for glycine); (b) the charge or hydrophobicity; and/or (c) the bulk of the side chain. By way of example and not limitation, exemplary non-conservative substitutions include an acidic amino acid substituted with a basic or aliphatic amino acid; an aromatic amino acid substituted with a small amino acid; and a hydrophilic amino acid substituted with a hydrophobic amino acid.

“Deletion” refers to modification to the polypeptide by removal of one or more amino acids from the reference polypeptide. Deletions can comprise removal of 1 or more amino acids, 2 or more amino acids, 5 or more amino acids, 10 or more amino acids, 15 or more amino acids, or 20 or more amino acids, up to 10% of the total number of amino acids, or up to 20% of the total number of amino acids making up the reference polypeptide while retaining enzymatic activity and/or retaining the improved properties of an engineered RNA ligase. Deletions can be directed to the internal portions and/or terminal portions of the polypeptide. In various embodiments, the deletion can comprise a continuous segment or can be discontinuous. As noted above, deletions are indicated by “-”, and may be present in substitution sets.

“Insertion” refers to modification to the polypeptide by addition of one or more amino acids from the reference polypeptide. Insertions can be in the internal portions of the polypeptide, or to the carboxy or amino terminus. Insertions as used herein include fusion proteins as is known in the art. The insertion can be a contiguous segment of amino acids or separated by one or more of the amino acids in the naturally occurring polypeptide.

“Functional fragment” and “biologically active fragment” are used interchangeably herein, to refer to a polypeptide that has an amino-terminal and/or carboxy-terminal deletion(s) and/or internal deletions, but where the remaining amino acid sequence is identical to the corresponding positions in the sequence to which it is being compared (e.g., a full length engineered RNA ligase of the present invention) and that retains substantially all of the activity of the full-length polypeptide.

“Isolated polypeptide” refers to a polypeptide which is substantially separated from other contaminants that naturally accompany it (e.g., protein, lipids, and polynucleotides). The term embraces polypeptides which have been removed or purified from their naturally-occurring environment or expression system (e.g., host cell or in vitro synthesis). The recombinant RNA ligase polypeptides may be present within a cell, present in the cellular medium, or prepared in various forms, such as lysates or isolated preparations. As such, in some embodiments, the recombinant RNA ligase polypeptides provided herein are isolated polypeptides.

“Substantially pure polypeptide” or “purified” refers to a composition in which the polypeptide species is the predominant species present (i.e., on a molar or weight basis it is more abundant than any other individual macromolecular species in the composition), and is generally a substantially purified composition when the object species comprises at least about 50 percent of the macromolecular species present by mole or % weight. Generally, a substantially pure RNA ligase composition will comprise about 60% or more, about 70% or more, about 80% or more, about 90% or more, about 95% or more, and about 98% or more of all macromolecular species by mole or % weight present in the composition. In some embodiments, the object species is purified to essential homogeneity (i.e., contaminant species cannot be detected in the composition by conventional detection methods) wherein the composition consists essentially of a single macromolecular species. Solvent species, small molecules (<500 Daltons), and elemental ion species are not considered macromolecular species. In some embodiments, the isolated recombinant RNA ligase polypeptides are substantially pure polypeptide compositions.

“Improved enzyme property” refers to an engineered RNA ligase polypeptide that exhibits an improvement in any enzyme property as compared to a reference RNA ligase polypeptide, such as a wild-type RNA ligase polypeptide or another engineered RNA ligase polypeptide. Improved properties include but are not limited to such properties as increased enzymatic activity, increased product yield, increased protein expression, increased thermoactivity, increased thermostability, increased stability, increased substrate specificity and/or affinity, increased substrate range, increased specific activity, increased resistance to substrate and/or end-product inhibition, increased chemical stability, improved solvent stability, increased solubility, and increased inhibitor resistance or tolerance. Exemplary improved properties are provided in the Examples.

“Increased enzymatic activity” and “enhanced catalytic activity” refer to an improved property of the engineered RNA ligase polypeptides, which can be represented by an increase in specific activity (e.g., product produced/time/weight protein) and/or an increase in percent conversion of the substrate to the product (e.g., percent conversion of starting amount of substrate to product in a specified time period using a specified amount of RNA ligase) as compared to the reference RNA ligase enzyme (e.g., wild-type RNA ligase and/or another engineered RNA ligase). Exemplary methods to determine enzyme activity are provided in the Examples. Any property relating to enzyme activity may be affected, including the classical enzyme properties of Kₘ, Vₘₐₓ or k_{cat}, changes of which can lead to increased enzymatic activity. Improvements in enzyme activity can be from about 1.1 fold the enzymatic activity of the corresponding wild-type enzyme, to about 1.5 fold, 2-fold, 5-fold, 10-fold, 20-fold, 25-fold, 50-fold, 75-fold, 100-fold, 150-fold, 200-fold or more enzymatic activity than the naturally occurring RNA ligase or another engineered RNA ligase from which the RNA ligase polypeptides were derived.

“Hybridization stringency” relates to hybridization conditions, such as washing conditions, in the hybridization of nucleic acids. Generally, hybridization reactions are performed under conditions of lower stringency, followed by washes of varying but higher stringency (see, e.g., Sambrook et al., Molecular Cloning: A Laboratory Manual, Cold Spring Harbor Laboratory Press, New York, 2001; Ausubel et al., Current Protocols in Molecular Biology, John Wiley & Sons, 2003). The term “moderately stringent hybridization” refers to conditions that permit target-DNA to bind a complementary nucleic acid that has about 60% identity, preferably about 75% identity, about 85% identity to the target DNA, with greater than about 90% identity to target-polynucleotide. Exemplary moderately stringent conditions are conditions equivalent to hybridization in 50% formamide, 5×Denhart's solution, 5×SSPE, 0.2% SDS at 42° C., followed by washing in 0.2×SSPE, 0.2% SDS, at 42° C. “High stringency hybridization” refers generally to conditions that are about 10° C. or less from the thermal melting temperature Tₘ as determined under the solution condition for a defined polynucleotide sequence. In some embodiments, a high stringency condition refers to conditions that permit hybridization of only those nucleic acid sequences that form stable hybrids in 0.018M NaCl at 65° C. (i.e., if a hybrid is not stable in 0.018M NaCl at 65° C., it will not be stable under high stringency conditions, as contemplated herein). High stringency conditions can be provided, for example, by hybridization in conditions equivalent to 50% formamide, 5×Denhart's solution, 5×SSPE, 0.2% SDS at 42° C., followed by washing in 0.1×SSPE, and 0.1% SDS at 65° C. Another high stringency condition comprises hybridizing in conditions equivalent to hybridizing in 5×SSC containing 0.1% (w: v) SDS at 65° C. and washing in 0.1×SSC containing 0.1% SDS at 65° C. Other high stringency hybridization conditions, as well as moderately stringent conditions, are described in the references cited above.

“Codon optimized” refers to changes in the codons of the polynucleotide encoding a protein to those preferentially used in a particular organism such that the encoded protein is more efficiently expressed in that organism. Although the genetic code is degenerate, in that most amino acids are represented by several codons, called “synonyms” or “synonymous” codons, it is well known that codon usage by particular organisms is nonrandom and biased towards particular codon triplets. This codon usage bias may be higher in reference to a given gene, genes of common function or ancestral origin, highly expressed proteins versus low copy number proteins, and the aggregate protein coding regions of an organism's genome. In some embodiments, the polynucleotides encoding the RNA ligase enzymes are codon optimized for optimal production from the host organism selected for expression.

“Control sequence” refers herein to include all components that are necessary or advantageous for the expression of a polynucleotide and/or polypeptide of the present disclosure. Each control sequence may be native or foreign (e.g., heterologous) to the nucleic acid sequence encoding the polypeptide. Such control sequences include, but are not limited to, leaders, polyadenylation sequences, propeptide sequences, promoter sequences, signal peptide sequences, initiation sequences, and transcription terminators. In some embodiments, the control sequences include a promoter, and transcriptional and translational stop signals. In some embodiments, the control sequences are provided with linkers for the purpose of introducing specific restriction sites facilitating ligation of the control sequences with the coding region of the nucleic acid sequence encoding a polypeptide.

“Operably linked” or “operatively linked” refers to a configuration in which a control sequence is appropriately placed (i.e., in a functional relationship) at a position relative to a polynucleotide of interest such that the control sequence directs or regulates the expression of the polynucleotide of interest, and where appropriate, expression of the encoded polypeptide of interest.

“Promoter” or “promoter sequence” refers to a nucleic acid sequence that is recognized by a host cell for expression of a polynucleotide of interest, such as a coding sequence. The promoter sequence contains transcriptional control sequences that mediate the expression of a polynucleotide of interest. The promoter may be any nucleic acid sequence which shows transcriptional activity in the host cell of choice including mutant, truncated, and hybrid promoters, and may be obtained from genes encoding extracellular or intracellular polypeptides either homologous or heterologous to the host cell.

“Suitable reaction conditions” or “suitable conditions” refers to those conditions in the enzymatic conversion reaction solution (e.g., ranges of enzyme loading, substrate loading, temperature, pH, buffers, co-solvents, etc.) under which an RNA ligase polypeptide of the present disclosure is capable of converting a polynucleotide(s) substrate to the desired ligated product polynucleotide. Exemplary “suitable reaction conditions” are provided herein (see, the Examples).

“Product” in the context of an enzymatic conversion process refers to the compound or molecule resulting from the action of the RNA ligase polypeptide on the substrate.

“Culturing” refers to the growing of a population of cells under suitable conditions using any suitable medium (e.g., liquid, gel, or solid).

“Vector” is a recombinant construct for introducing a polynucleotide of interest into a cell. In some embodiments, the vector is an expression vector that is operably linked to a suitable control sequence capable of effecting the expression in a suitable host of the polynucleotide or a polypeptide encoded in the polynucleotide. In some embodiments, an “expression vector” has a promoter sequence operably linked to the polynucleotide (e.g., transgene) to drive expression in a host cell, and in some embodiments, also comprises a transcription terminator sequence.

“Expression” includes any step involved in the production of the polypeptide including, but not limited to, transcription, post-transcriptional modification, translation, and post-translational modification. In some embodiments, the term also encompasses secretion of the polypeptide from a cell.

“Produces” refers to the production of proteins and/or other compounds by cells. It is intended that the term encompass any step involved in the production of polypeptides including, but not limited to, transcription, post-transcriptional modification, translation, and post-translational modification. In some embodiments, the term also encompasses secretion of the polypeptide from a cell.

“Heterologous” or “recombinant” refers to the relationship between two or more nucleic acid or polypeptide sequences (e.g., a promoter sequence, signal peptide, terminator sequence, etc.) that are derived from different sources and are not associated in nature.

“Host cell” and “host strain” refer to suitable hosts for expression vectors comprising a polynucleotide provided herein (e.g., a polynucleotide sequences encoding at least one RNA ligase variant). In some embodiments, the host cells are prokaryotic or eukaryotic cells that have been transformed or transfected with vectors constructed using recombinant DNA techniques, and progeny thereof, as known in the art.

“Analogue” in the context of a polypeptide means a polypeptide more than 70% sequence identity but less than 100% sequence identity (e.g., more than 75%, 78%, 80%, 83%, 85%, 88%, 90%, 91%, 92%, 93%, 94%, 95%, 96%, 97%, 98%, 99% sequence identity) with a reference polypeptide. In some embodiments, analogues include non-naturally occurring amino acid residues including, but not limited, to homoarginine, ornithine and norvaline, as well as naturally occurring amino acids. In some embodiments, analogues also include one or more D-amino acid residues and non-peptide linkages between two or more amino acid residues.

### Engineered RNA Ligase Polypeptides

In one aspect, the present disclosure provides RNA ligases engineered to have an improved property, particularly increased activity and increased product yield. In some embodiments, the increased activity and increased product yield is on ligation of modified polynucleotides, including polynucleotide substrates with 2′-modified sugar moieties and/or phosphorothioate internucleoside linkages.

In some embodiments, the engineered RNA ligase, or a functional fragment thereof, comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to a reference sequence corresponding to residues 12 to 346 of an even-numbered SEQ ID NO. of SEQ ID NOs: 2-1470 and 1478-3058, or to a reference sequence corresponding to an even-numbered SEQ ID NO. of SEQ ID NOs: 2-1470 and 1478-3058, wherein the amino acid sequence comprises one or more substitutions relative to a reference sequence corresponding to residues 12 to 346 of SEQ ID NO: 2, 54, 244, 648, 862, 956, 1088, 1416, 1612, 1840, 1866, 1954, 2062, 2674, 2754, or 2786, or relative to a reference sequence corresponding to SEQ ID NO: 2, 54, 244, 648, 862, 956, 1088, 1416, 1612, 1840, 1866, 1954, 2062, 2674, 2754, or 2786.

In some embodiments, the engineered RNA ligase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12 to 346 of SEQ ID NO: 2, 54, 244, 648, 862,956, 1088, 1416, 1612, 1840, 1866, 1954, 2062, 2674, 2754, or 2786, or to the reference sequence corresponding to SEQ ID NO: 2, 54, 244, 648, 862, 956, 1088, 1416, 1612, 1840, 1866, 1954, 2062, 2674, 2754, or 2786, wherein the amino acid sequence comprises one or more substitutions relative to the reference sequence corresponding to residues 12 to 346 of SEQ ID NO: 2, 54, 244, 648, 862,956, 1088, 1416, 1612, 1840, 1866, 1954, 2062, 2674, 2754, or 2786, or relative to the reference sequence corresponding to SEQ ID NO: 2, 54, 244, 648, 862, 956, 1088, 1416, 1612, 1840, 1866, 1954, 2062, 2674, 2754, or 2786.

In some embodiments, the engineered RNA ligase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12 to 346 of SEQ ID NO: 2, or to the reference sequence corresponding to SEQ ID NO: 2, wherein the amino acid sequence comprises one or more substitutions relative to the reference sequence corresponding to residues 12 to 346 of SEQ ID NO: 2, or relative to the reference sequence corresponding to SEQ ID NO: 2.

In some embodiments, the engineered RNA ligase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12 to 346 of SEQ ID NO: 54, 244, 648, 862, 956, 1088, 1416, 1612, 1840, 1866, 1954, 2062, 2674, 2754, or 2786, or to the reference sequence corresponding to SEQ ID NO: 54, 244, 648, 862, 956, 1088, 1416, 1612, 1840, 1866, 1954, 2062, 2674, 2754, or 2786, wherein the amino acid sequence comprises one or more substitutions relative to the reference sequence corresponding to residues 12 to 346 of SEQ ID NO: 2, or relative to the reference sequence corresponding to SEQ ID NO: 2.

In some embodiments, the engineered RNA ligase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12 to 346 of an even-numbered SEQ ID NO. of SEQ ID NOs: 4-1470 and 1478-3058, or to the reference sequence corresponding to an even-numbered SEQ ID NO. of SEQ ID NOs: 4-1470 and 1478-3058, wherein the amino acid sequence comprises one or more substitutions relative to the reference sequence corresponding to residues 12 to 346 of SEQ ID NO: 2, or relative to the reference sequence corresponding to SEQ ID NO: 2.

In some embodiments, the engineered RNA ligase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12 to 346 of an even-numbered SEQ ID NO. of SEQ ID NOs: 4-218, or to the reference sequence corresponding to an even-numbered SEQ ID NO. of SEQ ID NOs: 4-218, wherein the amino acid sequence comprises one or more substitutions relative to the reference sequence corresponding to residues 12 to 346 of SEQ ID NO: 2, or relative to the reference sequence corresponding to SEQ ID NO: 2.

In some embodiments, the amino acid sequence of the engineered RNA ligase comprises at least a substitution at amino acid position 10, 11, 12, 13, 15, 17, 18, 21, 23, 25, 26, 28, 29, 30, 32, 33, 37, 38, 39, 40, 41, 42, 45, 52, 54, 56, 57, 58, 60, 61, 62, 64, 65, 67, 71, 73, 74, 75, 77, 80, 81, 83, 84, 86, 87, 89, 90, 91, 93, 95, 97, 99, 100, 101, 102, 103, 104, 105, 106, 107, 109, 113, 114, 116, 117, 119, 121, 122, 123, 125, 126, 127, 128, 129, 131, 135, 137, 140, 141, 143, 144, 148, 150, 154, 156, 157, 158, 159, 160, 161, 162, 165, 169, 170, 171, 173, 175, 177, 178, 179, 181, 183, 184, 185, 190, 191, 192, 193, 194, 197, 198, 204, 206, 208, 210, 212, 213, 214, 215, 218, 223, 224, 226, 228, 229, 230, 232, 236, 238, 241, 243, 247, 248, 249, 250, 251, 252, 253, 254, 255, 256, 257, 258, 260, 261, 264, 265, 266, 267, 268, 270, 271, 272, 274, 276, 277, 280, 282, 283, 286, 288, 289, 290, 291, 292, 294, 295, 296, 297, 298, 299, 300, 301, 302, 305, 306, 307, 310, 311, 315, 316, 317, 318, 323, 324, 325, 326, 327, 330, 331, 332, 334, 335, 336, 337, 338, 341, 342, 344, 345, or 346, or any combinations thereof, wherein the amino acid positions are relative to the reference sequence corresponding to residues 12 to 346 of SEQ ID NO: 2, or relative to the reference sequence corresponding to SEQ ID NO: 2.

In some embodiments, the amino acid sequence of the engineered RNA ligase comprises at least a substitution or amino acid residue 10F, 11G/N, 12A/F/L/M/P/R/T/V, 13W, 15M, 17P, 18A/H, 21S, 23K/T, 25A/D/F/G/L/M/S/T, 26L, 28A/L/M/V, 29D/S, 30A/L/T/V/W, 32E, 33A/I/L/T/V, 37A/F/K/L/M/R, 38A/E/L/R/S/T/V, 39P, 40D/I/P/S, 41E/I, 42T, 45S, 52C, 54L, 56I/L, 57V, 58M, 60A/C/D/R/S/V, 61A, 62A/V, 64A/Q/W, 65C, 67R, 71T, 73Q/W, 74D, 75A/D, 77M, 80E, 81Y, 83A/E/G/L/T/V, 84V, 86D/G/L, 87E, 89C/D/E/G/L/M/N/Q/R/S/T/V/W/Y, 90E/F, 91A/E/V, 93K/V, 95A/C/G/I, 97A/E, 99I, 100A/G/H/L/R/S/T/V, 101P/V/W, 102T, 103G/V, 104T, 105K/L/R, 106G/L/M/Q/R/V/W, 107M/S, 109F/L, 113L/M, 114E, 116L, 117A/Q, 119L, 121N, 122C/F/G/L/Q/T/W, 123K/R, 125K/T, 126M, 127P, 128E, 129L/W, 131T, 135A/H/M/Q/V, 137N/R, 140I, 141D/G, 143A/C/E/F/H/M/S/T/V, 144L, 148F, 150D/L, 154S/V, 156G/H/Q/S, 157F/L/Q/V, 158R/S, 159F, 160R, 161M/V, 162C, 165M, 169G/N, 170H/Y, 171S, 173M, 175A, 177K, 178V, 179E, 181E/N/P/R/S, 183L/V, 184L, 185C/F/G/K/M/P/S/T/V, 190I/M, 191C, 192D/E/F/L/N, 193K/Q, 194A/G/V, 197W, 198M, 204W, 206L, 208H/I/K/L/P/T, 210G/V, 212E/G/I/K/L, 213D/N/R/T, 214A/C/H/K/L/Q/Y, 215G/T, 218F, 223C, 224A/Q/R, 226R, 228D/L, 229W, 230T/V, 232G/M/N/R/S, 236L/V, 238K/S/V, 241E/G/Q/R, 243H, 247I/L/R/S, 248F/G/I/K/T, 249K/L/R/S, 250E/I/S, 251C/E/F/G/L/R/S/T/Y, 252A/Q, 253L/W, 254E/K/L/N/Q/S/V, 255N/S/V/Y, 256A/E/G/M/S/W, 257D/E/G, 258D/M/P/V, 260G, 261D/P/T, 264A/E/G/S/V, 265A/F, 266C/I/L, 267I/M, 268F/G/M, 270L/M/V, 271A/E/G/T, 272M/S, 274A, 276A/G/M/V, 277A, 280L/N/W, 282A/M, 283C/V, 286D/E/F/M/V, 288E/L/R/W, 289A/D/E/I/P/T, 290D/H/N/P, 291H/L/Q/R/S/T/W, 292N/S, 294A/S/V/Y, 295C/F/G/K/L/M/P/Q/R/S/T/V/Y, 296E/G/L/Q/W, 297A/I/L, 298C/I/K/R/S/V, 299E/I/K/Q/R/T/V, 300A, 301A/F/I/L/M/V, 302A/C/I/L/S, 305V, 306G/L/T, 307K/R, 310H/L/M/N/Q/R/S/T, 311C, 315E/S, 316I/L/Y, 317E/F/G/Q/V/W, 318A/E/G/S, 323E/G, 324D/G/M, 325L, 326C/L, 327E/G/M/S, 330A/D/E/G/K/R, 331D/G/R/S/W, 332E/H/W, 334E/R, 335H/Q, 336I/L/M, 337A/V, 338G/L/M, 341A, 342R/S/T, 344K, 345Q/S, or 346S, or any combinations thereof, wherein the amino acid positions are relative to the reference sequence corresponding to residues 12 to 346 of SEQ ID NO: 2, or relative to the reference sequence corresponding to SEQ ID NO: 2.

In some embodiments, the amino acid sequence of the engineered RNA ligase comprises at least a substitution or amino acid residue S10F, G11N, M12A/F/L/P/R/T/V, F13W, Y15M, S17P, S18A/H, N21S, Y23K/T, N25A/D/F/G/L/M/S/T, K26L, I28A/L/M/V, E29D/S, K30A/L/T/V/W, R32E, G33A/I/L/T/V, D37A/F/K/L/M/R, M38A/E/L/R/S/T/V, H39P, T40D/I/P/S, V41E/I, E42T, A45S, T52C, F54L, V56I/L, I57V, I58M, P60A/C/D/R/S/V, T61A, E62A/V, V64A/Q/W, P65C, K67R, P71T, L73Q/W, E74D, G75A/D, S77M, G80E, H81Y, 183A/E/G/L/T/V, 184V, K86D/G/L, K87E, K89C/D/E/G/L/M/N/Q/R/S/T/V/W/Y, D90E/F, S91A/E/V, V93K, V95A/C/G/I, N97A/E, L99I, N100A/G/H/L/R/S/T/V, A101P/V/W, M102T, D103G/V, L104T, V105K/L/R, S106G/L/M/Q/R/V/W, V107M/S, I109F/L, F113L/M, A114E, G116L, G117A/Q, Q119L, G121N, V122C/F/G/L/Q/T/W, D123K/R, G125K/T, D126M, K127P, D128E, F129L/W, V131T, L135A/H/M/Q/V, D137N/R, N140I, E141D/G, S143A/C/E/F/H/M/T/V, Y144L, Y148F, V150D/L, C154S/V, T156G/H/Q/S, L157F/Q/V, G158R/S, L159F, K160R, L161M/V, A162C, L165M, S169G/N, F170H/Y, A171S, L173M, Q175A, P177K, N178V, D179E, K181E/N/P/R/S, I183L/V, V184L, N185C/F/G/K/M/P/S/T/V, L190I/M, V191C, D192E/F/L/N, T193K/Q, T194A/G/V, E197W, H198M, F204W, P206L, A208H/I/K/L/P/T, S210G/V, T212E/G/I/K/L, G213D/N/R/T, L214A/C/H/K/L/Q/Y, A215G/T, Y218F, V223C, Y224A/Q/R, I226R, F228D/L, N229W, N230T/V, T232G/M/N/R/S, 1236L/V, C238K/S/V, S241E/G/Q/R, F243H, A247I/L/R/S, F248G/I/K/T, L249K/L/R/S, D250E/I/S, K251C/E/F/G/L/R/S/T/Y, P252A/Q, I253L/W, K254E/L/N/Q/S/V, A255N/S/V/Y, K256A/E/G/M/S/W, V257D/E/G, F258D/M/P/V, V260G, E261D/P/T, K264A/E/G/S/V, R265A/F, V266C/I/L, L267I/M, E268F/G/M, F270L/M/V, S271A/E/G/T, E272M/S, V274A, W276A/G/M/V, N277A, S280L/N/W, V282A/M, L283C/V, I286D/E/F/M/V, S288E/L/R/W, V289A/D/E/I/P/T, T290D/H/N/P, A291H/L/Q/R/S/T/W, A292N/S, F294A/S/V/Y, G295C/F/K/L/M/P/Q/R/S/T/V/Y, R296E/G/L/Q/W, V297A/I/L, M298C/I/K/R/S/V, G299E/I/K/Q/R/T/V, L300A, T301A/F/I/L/M/V, M302A/C/I/L/S, 1305V, I306G/L/T, N307K/R, A310H/L/M/N/Q/R/S/T, R311C, D315E/S, L316I/L/Y, L317E/F/G/Q/V/W, F318A/E/G/S, S323E/G, A324D/G/M, V325L, K326C/L, K327E/G/M/S, T330A/D/E/G/K/R, I331D/G/R/S/W, L332E/H/W, Q334E/R, N335H/Q, T336I/L/M, M337A/V, R338G/L/M, W341A, H342R/S/T, V344K, L345Q/S, or E346S, or any combinations thereof, wherein the amino acid positions are relative to the reference sequence corresponding to residues 12 to 346 of SEQ ID NO: 2, or relative to the reference sequence corresponding to SEQ ID NO: 2.

In some embodiments, the amino acid sequence of the engineered RNA ligase comprises at least a substitution at amino acid position 10, 11, 12, 13, 17, 21, 23, 25, 26, 28, 29, 30, 32, 37, 38, 40, 41, 42, 45, 52, 54, 56, 57, 58, 60, 61, 62, 64, 65, 67, 71, 73, 74, 75, 77, 80, 83, 84, 87, 90, 91, 93, 97, 99, 100, 101, 102, 103, 104, 105, 106, 107, 109, 113, 114, 121, 122, 123, 125, 126, 127, 128, 129, 131, 135, 137, 140, 141, 143, 148, 150, 157, 158, 160, 161, 162, 165, 169, 170, 173, 178, 179, 184, 190, 191, 192, 193, 194, 197, 198, 204, 206, 208, 210, 213, 215, 218, 223, 228, 229, 232, 236, 238, 241, 243, 248, 249, 250, 251, 252, 253, 254, 255, 258, 261, 264, 265, 267, 268, 270, 271, 272, 274, 276, 277, 282, 286, 289, 290, 292, 294, 295, 297, 298, 299, 300, 301, 302, 305, 311, 318, 323, 324, 325, 327, 332, 336, 338, 341, 344, or 346; a substitution or amino acid residue 18A/H, 33A/I/T/V, 39P, 86D/L, 89C/D/E/L/N/Q/R/S/T/V/Y, 95A/C/G/I, 116L, 117A/Q, 119L, 144L, 154S/V, 156G/H/S, 171S, 175A, 177K, 181E/N/P/R/S, 183L, 212E/G/K/L, 224A/Q/R, 226R, 230T/V, 247I/L/R/S, 256E/G/M/S/W, 257D/E/G, 260G, 280L/W, 283C/V, 288L/R/W, 291S, 296E/G/L/Q/W, 306G/T, 307K/R, 310H/L/M/N/Q/R/S/T, 315E, 316I/Y, 317E/F/G/Q/V/W, 326C/L, 330A/D/E/G/K, 331D/G/S, 334E, 335Q, 337A/V, 338G/L/M, 342R/S/T, or 345Q/S; or any combinations thereof, wherein the amino acid positions are relative to the reference sequence corresponding to residues 12 to 346 of SEQ ID NO: 2, or relative to the reference sequence corresponding to SEQ ID NO: 2.

In some embodiments, the substitution at amino acid position 10, 11, 12, 13, 17, 21, 23, 25, 26, 28, 29, 30, 32, 37, 38, 40, 41, 42, 45, 52, 54, 56, 57, 58, 60, 61, 62, 64, 65, 67, 71, 73, 74, 75, 77, 80, 83, 84, 87, 90, 91, 93, 97, 99, 100, 101, 102, 103, 104, 105, 106, 107, 109, 113, 114, 121, 122, 123, 125, 126, 127, 128, 129, 131, 135, 137, 140, 141, 143, 148, 150, 157, 158, 160, 161, 162, 165, 169, 170, 173, 178, 179, 184, 190, 191, 192, 193, 194, 197, 198, 204, 206, 208, 210, 213, 215, 218, 223, 228, 229, 232, 236, 238, 241, 243, 248, 249, 250, 251, 252, 253, 254, 255, 258, 261, 264, 265, 267, 268, 270, 271, 272, 274, 276, 277, 282, 286, 289, 290, 292, 294, 295, 297, 298, 299, 300, 301, 302, 305, 311, 318, 323, 324, 325, 327, 332, 336, 338, 341, 344, or 346 is 10F, 11N, 12A/F/L/M/P/R/T/V, 13W, 17P, 21S, 23K/T, 25A/D/F/G/L/M/S/T, 26L, 28A/L/M/V, 29D/S, 30A/L/T/V/W, 32E, 37A/F/K/L/M/R, 38A/E/L/R/S/T/V, 40D/I/P/S, 41E/I, 42T, 45S, 52C, 54L, 56I/L, 57V, 58M, 60A/C/D/R/S/V, 61A, 62A/V, 64A/Q/W, 65C, 67R, 71T, 73Q/W, 74D, 75A/D, 77M, 80E, 83A/E/G/L/T/V, 84V, 87E, 90E/F, 91A/E/V, 93K, 97A/E, 991, 100A/G/H/L/R/S/T/V, 101P/V/W, 102T, 103G/V, 104T, 105K/L/R, 106G/L/M/Q/R/V/W, 107M/S, 109F/L, 113L/M, 114E, 121N, 122C/F/G/L/Q/T/W, 123K/R, 125K/T, 126M, 127P, 128E, 129L/W, 131T, 135A/H/M/Q/V, 137N/R, 140I, 141D/G, 143A/C/E/F/H/M/T/V, 148F, 150D/L, 157F/Q/V, 158R/S, 160R, 161M/V, 162C, 165M, 169G/N, 170H/Y, 173M, 178V, 179E, 184L, 190I/M, 191C, 192E/F/L/N, 193K/Q, 194A/G/V, 197W, 198M, 204W, 206L, 208H/I/K/L/P/T, 210G/V, 212E/G/K/L, 213D/N/R/T, 214A/C/H/K/Q/Y, 215G/T, 218F, 223C, 228D/L, 229W, 232G/M/N/R/S, 236L/V, 238K/S/V, 241E/G/Q/R, 243H, 248G/I/K/T, 249K/L/R/S, 250E/I/S, 251C/E/F/G/L/R/S/T/Y, 252A/Q, 253L/W, 254E/L/N/Q/S/V, 255N/S/V/Y, 258D/M/P/V, 264A/E/G/S/V, 265A/F, 266C/I, 267I/M, 268F/G/M, 270L/M/V, 271A/E/G/T, 272M/S, 274A, 276A/G/M/V, 277A, 282A/M, 286D/E/F/M/V, 289A/D/E/I/P/T, 290D/H/N/P, 291H/L/Q/R/S/T/W, 292N/S, 294A/S/V/Y, 295C/F/K/L/M/P/Q/R/S/T/V/Y, 297A/I/L, 298I/S, 298C/K/R/V, 299E/I/K/Q/R/T/V, 300A, 301A/F/I/L/M/V, 302A/C/I/L/S, 305V, 311C, 318A/E/G/S, 323E/G, 324D/G/M, 325L, 332E/H/W, 336I/L/M, 338G/L/M, 341A, 344K, or 346S, respectively.

In some embodiments, the amino acid sequence of the engineered RNA ligase comprises at least a substitution or amino acid residue 10F, 11N, 12A/F/L/M/P/R/T/V, 13W, 17P, 18A/H, 21S, 23K/T, 25A/D/F/G/L/M/S/T, 26L, 28A/L/M/V, 29D/S, 30A/L/T/V/W, 32E, 33A/I/T/V, 37A/F/K/L/M/R, 38A/E/L/R/S/T/V, 39P, 40D/I/P/S, 41E/I, 42T, 45S, 52C, 54L, 56I/L, 57V, 58M, 60A/C/D/R/S/V, 61A, 62A/V, 64A/Q/W, 65C, 67R, 71T, 73Q/W, 74D, 75A/D, 77M, 80E, 83A/E/G/L/T/V, 84V, 86D/L, 87E, 89C/D/E/L/N/Q/R/S/T/V/Y,90E/F,91A/E/V, 93K, 95A/C/G/I, 97A/E, 991, 100A/G/H/L/R/S/T/V, 101P/V/W, 102T, 103G/V, 104T, 105K/L/R, 106G/L/M/Q/R/V/W, 107M/S, 109F/L, 113L/M, 114E, 116L, 117A/Q, 119L, 121N, 122C/F/G/L/Q/T/W, 123K/R, 125K/T, 126M, 127P, 128E, 129L/W, 131T, 135A/H/M/Q/V, 137N/R, 140I, 141D/G, 143A/C/E/F/H/M/T/V, 144L, 148F, 150D/L, 154S/V, 156G/H/S, 157F/Q/V, 158R/S, 160R, 161M/V, 162C, 165M, 169G/N, 170H/Y, 171S, 173M, 175A, 177K, 178V, 179E, 181E/N/P/R/S, 183L, 184L, 185C/F/M/P/S/T/V, 190I/M, 191C, 192E/F/L/N, 193K/Q, 194A/G/V, 197W, 198M, 204W, 206L, 208H/I/K/L/P/T, 210G/V, 212E/G/K/L, 213D/N/R/T, 214A/C/H/K/Q/Y, 215G/T, 218F, 223C, 224A/Q/R, 226R, 228D/L, 229W, 230T/V, 232G/M/N/R/S, 236L/V, 238K/S/V, 241E/G/Q/R, 243H, 247I/L/R/S, 248G/I/K/T, 249K/L/R/S, 250E/I/S, 251C/E/F/G/L/R/S/T/Y, 252A/Q, 253L/W, 254E/L/N/Q/S/V, 255N/S/V/Y, 256E/G/M/S/W, 257D/E/G, 258D/M/P/V, 260G, 261D/P/T, 264A/E/G/S/V, 265A/F, 266C/I, 267I/M, 268F/G/M, 270L/M/V, 271A/E/G/T, 272M/S, 274A, 276A/G/M/V, 277A, 280L/W, 282A/M, 283C/V, 286D/E/F/M/V,288L/R/W, 289A/D/E/I/P/T, 290D/H/N/P, 291H/L/Q/R/S/T/W, 291S, 292N/S, 294A/S/V/Y, 295C/F/K/L/M/P/Q/R/S/T/V/Y, 296E/G/L/Q/W, 297A/I/L, 298I/S, 298C/K/R/V, 299E/I/K/Q/R/T/V, 300A, 301A/F/I/L/M/V, 302A/C/I/L/S, 305V, 306G/T, 307K/R, 310H/L/M/N/Q/R/S/T, 311C, 315E, 316I/Y, 317E/F/G/Q/V/W, 318A/E/G/S, 323E/G, 324D/G/M, 325L, 326C/L, 327E/G/M/S, 330A/D/E/G/K, 331D/G/S, 332E/H/W, 334E, 335Q, 336I/L/M, 337A/V, 338G/L/M, 341A, 342R/S/T, 344K, 345Q/S, or 346S, or any combinations thereof, wherein the amino acid positions are relative to the reference sequence corresponding to residues 12 to 346 of SEQ ID NO: 2, or relative to the reference sequence corresponding to SEQ ID NO: 2.

In some embodiments, the amino acid sequence of the engineered RNA ligase comprises at least a substitution or amino acid residue S10F, G11N, M12A/F/L/P/R/T/V, F13W, Y15M, S17P, S18A/H, N21S, Y23K/T, N25A/D/F/G/L/M/S/T, K26L, I28A/L/M/V, E29D/S, K30A/L/T/V/W, R32E, G33A/I/L/T/V, D37A/F/K/L/M/R, M38A/E/L/R/S/T/V, H39P, T40D/I/P/S, V41E/I, E42T, A45S, T52C, F54L, V56I/L, I57V, 158M, P60A/C/D/R/S/V, T61A, E62A/V, V64A/Q/W, P65C, K67R, P71T, L73Q/W, E74D, G75A/D, S77M, G80E, H81Y, I83A/E/G/L/T/V, 184V, K86D/G/L, K87E, K89C/D/E/G/L/M/N/Q/R/S/T/V/W/Y, D90E/F, S91A/E/V, V93K, V95A/C/G/I, N97A/E, L99I, N100A/G/H/L/R/S/T/V, A101P/V/W, M102T, D103G/V, L104T, V105K/L/R, S106G/L/M/Q/R/V/W, V107M/S, I109F/L, F113L/M, A114E, G116L, G117A/Q, Q119L, G121N, V122C/F/G/L/Q/T/W, D123K/R, G125K/T, D126M, K127P, D128E, F129L/W, V131T, L135A/H/M/Q/V, D137N/R, N140I, E141D/G, S143A/C/E/F/H/M/T/V, Y144L, Y148F, V150D/L, C154S/V, T156G/H/Q/S, L157F/Q/V, G158R/S, L159F, K160R, L161M/V, A162C, L165M, S169G/N, F170H/Y, A171S, L173M, Q175A, P177K, N178V, D179E, K181E/N/P/R/S, I183L/V, V184L, N185C/F/G/K/M/P/S/T/V, L190I/M, V191C, D192E/F/L/N, T193K/Q, T194A/G/V, E197W, H198M, F204W, P206L, A208H/I/K/L/P/T, S210G/V, T212E/G/K/L, G213D/N/R/T, L214A/C/H/K/L/Q/Y, A215G/T, Y218F, V223C, Y224A/Q/R, I226R, F228D/L, N229W, N230T/V, T232G/M/N/R/S, I236L/V, C238K/S/V, S241E/G/Q/R, F243H, A247I/L/R/S, F248G/I/K/T, L249K/L/R/S, D250E/I/S, K251C/E/F/G/L/R/S/T/Y, P252A/Q, I253L/W, K254E/L/N/Q/S/V, A255N/S/V/Y, K256A/E/G/M/S/W, V257D/E/G, F258D/M/P/V, V260G, E261D/P/T, K264A/E/G/S/V, R265A/F, V266C/I/L, L267I/M, E268F/G/M, F270L/M/V, S271A/E/G/T, E272M/S, V274A, W276A/G/M/V, N277A, S280L/N/W, V282A/M, L283C/V, I286D/E/F/M/V, S288E/L/R/W, V289A/D/E/I/P/T, T290D/H/N/P, A291H/L/Q/R/S/T/W, A292N/S, F294A/S/V/Y, G295C/F/K/L/M/P/Q/R/S/T/V/Y, R296E/G/L/Q/W, V297A/I/L, M298C/I/K/R/S/V, G299E/I/K/Q/R/T/V, L300A, T301A/F/I/L/M/V, M302A/C/I/L/S, I305V, 1306G/L/T, N307K/R, A310H/L/M/N/Q/R/S/T, R311C, D315E/S, L316I/L/Y, L317E/F/G/Q/V/W, F318A/E/G/S, S323E/G, A324D/G/M, V325L, K326C/L, K327E/G/M/S, T330A/D/E/G/K/R, I331D/G/R/S/W, L332E/H/W, Q334E/R, N335H/Q, T336I/L/M, M337A/V, R338G/L/M, W341A, H342R/S/T, V344K, L345Q/S, E346S, or any combinations thereof, wherein the amino acid positions are relative to the reference sequence corresponding to residues 12 to 346 of SEQ ID NO: 2, or relative to the reference sequence corresponding to SEQ ID NO: 2.

In some embodiments, the amino acid sequence of the engineered RNA ligase comprises at least a substitution at amino acid position 11, 12, 23, 25, 29, 37, 38, 75, 86, 89, 93, 101, 122, 143, 157, 183, 192, 194, 198, 212, 214, 230, 241, 248, 249, 250, 251, 254, 266, 268, 280, 291, 295, 298, 307, 316, 318, 326, 330, or 331, or any combinations thereof, wherein the amino acid positions are relative to the reference sequence corresponding to residues 12 to 346 of SEQ ID NO: 2, or relative to the reference sequence corresponding to SEQ ID NO: 2.

In some embodiments, the amino acid sequence of the engineered RNA ligase comprises at least a substitution or amino acid residue 11N, 12A/F/L/P/R/T/V, 23K/T, 25A/D/F/G/L/M/S/T, 29D/S, 37A/F/K/L/M/R, 38A/E/L/R/S/T/V, 75A/D, 86D/L, 89C/D/E/L/N/Q/R/S/T/V/Y, 93K, 101P/V/W, 122C/F/G/L/Q/T/W, 143A/C/E/F/H/M/T/V, 157F/Q/V, 183L/V, 192E/F/L/N, 194A/G/V, 198M, 212E/G/I/K/L, 214A/C/H/K/L/Q/Y, 230T/V, 241E/G/Q/R, 248G/I/K/T, 249K/L/R/S, 250E/I/S, 251C/E/F/G/L/R/S/T/Y, 254E/K/L/N/Q/S/V, 266C/I/L, 268F/G/M, 280L/N/W, 291H/L/Q/R/S/T/W, 295C/F/K/L/M/P/Q/R/S/T/V/Y,298C/I/K/R/S/V, 307K/R, 316I/L/Y, 318A/E/G/S, 326C/L, 330A/D/E/G/K/R, or 331D/G/R/S/W, or any combinations thereof, wherein the amino acid positions are relative to the reference sequence corresponding to residues 12 to 346 of SEQ ID NO: 2, or relative to the reference sequence corresponding to SEQ ID NO: 2.

In some embodiments, the amino acid sequence of the engineered RNA ligase comprises at least a substitution or amino acid residue 11N, 12T, 23T, 25A, 29S, 37L, 38A/S, 75D, 86D, 89M, 93K, 101P, 122F, 157V, 183L, 192E, 194V, 198M, 212E, 214L, 226R, 230T/V, 241G, 249L, 250I, 251E/S, 254S, 266L, 268G, 280L, 291Q, 295V, 298S/V, 307R, 316L, 318S, 326L, 330A, or 331G, or any combinations thereof, wherein the amino acid positions are relative to the reference sequence corresponding to residues 12 to 346 of SEQ ID NO: 2, or relative to the reference sequence corresponding to SEQ ID NO: 2.

In some embodiments, the amino acid sequence of the engineered RNA ligase comprises at least a substitution at amino acid position 11, 12, 23, 25, 29, 37, 38, 75, 93, 101, 122, 143, 157, 192, 194, 198, 241, 248, 249, 250, 251, 254, 268, 295, 298, or 318; a substitution or amino acid residue 86D/L, 89C/D/E/L/N/Q/R/S/T/V/Y, 183L, 212E/G/K/L, 214A/C/H/K/L/Q/Y, 226R, 230T/V, 266C/1, 280L/W, 291H/L/Q/R/S/T/W, 307K/R, 326C/L, 330A/D/E/G/K, or 331D/G/S; or any combinations thereof, wherein the amino acid positions are relative to the reference sequence corresponding to residues 12 to 346 of SEQ ID NO: 2, or relative to the reference sequence corresponding to SEQ ID NO: 2.

In some embodiments, the amino acid sequence of the engineered RNA ligase comprises at least a substitution or amino acid residue 11N, 12A/F/L/P/R/T/V, 23K/T, 25A/D/F/G/L/M/S/T, 29D/S, 37A/F/K/L/M/R, 38A/E/L/R/S/T/V, 75A/D, 86D/L, 89C/D/E/L/N/Q/R/S/T/V/Y, 93K, 101P/V/W, 122C/F/G/L/Q/T/W, 143A/C/E/F/H/M/T/V, 157F/Q/V, 183L, 192E/F/L/N, 194A/G/V, 198M, 212E/G/K/L, 214A/C/H/K/L/Q/Y, 226R, 230T/V, 241E/G/Q/R, 248G/I/K/T, 249K/L/R/S, 250E/I/S, 251C/E/F/G/L/R/S/T/Y, 254E/L/N/Q/S/V, 266C/1, 268F/G/M, 280L/W, 291H/L/Q/R/S/T/W, 295C/F/K/L/M/P/Q/R/S/T/V/Y, 298C/K/R/V, 307K/R, 318A/E/G/S, 326C/L, 330A/D/E/G/K, or 331D/G/S, or any combinations thereof, wherein the amino acid positions are relative to the reference sequence corresponding to residues 12 to 346 of SEQ ID NO: 2, or relative to the reference sequence corresponding to SEQ ID NO: 2.

In some embodiments, the amino acid sequence of the engineered RNA ligase comprises at least a substitution or amino acid residue 11N, 12T, 23T, 25A, 29S, 37L, 38A/S, 75D, 86D, 93K, 101P, 122F, 157V, 183L, 192E, 194V, 198M, 212E, 214L, 226R, 230T/V, 241G, 249L, 2501, 251E/S, 254S, 266L, 268G, 280L, 291Q, 295V, 298S/V, 307R, 318S, 326L, 330A, or 331G, or any combinations thereof, wherein the amino acid positions are relative to the reference sequence corresponding to residues 12 to 346 of SEQ ID NO: 2, or relative to the reference sequence corresponding to SEQ ID NO: 2.

In some embodiments, the amino acid sequence of the engineered RNA ligase comprises at least a substitution at amino acid position 11, 37, 249, 266, or 316 or any combinations thereof, wherein the amino acid positions are relative to the reference sequence corresponding to residues 12 to 346 of SEQ ID NO: 2, or relative to the reference sequence corresponding to SEQ ID NO: 2. In some embodiments, the amino acid sequence of the engineered RNA ligase comprises at least a substitution or amino acid residue 11N, 37L, 249L, 266L, or 316L, or any combinations thereof, wherein the amino acid positions are relative to the reference sequence corresponding to residues 12 to 346 of SEQ ID NO: 2, or relative to the reference sequence corresponding to SEQ ID NO: 2. In some embodiments, the amino acid sequence of the engineered RNA ligase comprises at least a substitution G11N, D37L, S249L, V266L, or M316L, or any combinations thereof, wherein the amino acid positions are relative to the reference sequence corresponding to residues 12 to 346 of SEQ ID NO: 2, or relative to the reference sequence corresponding to SEQ ID NO: 2. In some embodiments, the amino acid sequence of the engineered RNA ligase comprises at least a substitution set at amino acid positions 11/37/249/266/316, wherein the amino acid positions are relative to the reference sequence corresponding to residues 12 to 346 of SEQ ID NO: 2, or relative to the reference sequence corresponding to SEQ ID NO: 2. In some embodiments, the amino acid sequence of the engineered RNA ligase comprises at least a substitution set 11N/37L/249L/266L/316L, wherein the amino acid positions are relative to the reference sequence corresponding to residues 12 to 346 of SEQ ID NO: 2, or relative to the reference sequence corresponding to SEQ ID NO: 2.

In some embodiments, the amino acid sequence of the engineered RNA ligase comprises at least a substitution at amino acid position 12, 230, or 251, or any combinations thereof, wherein the amino acid positions are relative to the reference sequence corresponding to residues 12 to 346 of SEQ ID NO: 2, or relative to the reference sequence corresponding to SEQ ID NO: 2. In some embodiments, the amino acid sequence of the engineered RNA ligase comprises at least a substitution or amino acid residue 12T, 230T, or 251S, or any combinations thereof, wherein the amino acid positions are relative to the reference sequence corresponding to residues 12 to 346 of SEQ ID NO: 2, or relative to the reference sequence corresponding to SEQ ID NO: 2. In some embodiments, the amino acid sequence of the engineered RNA ligase comprises at least a substitution M12T, N230T, or K251S, or any combinations thereof, wherein the amino acid positions are relative to the reference sequence corresponding to residues 12 to 346 of SEQ ID NO: 2, or relative to the reference sequence corresponding to SEQ ID NO: 2. In some embodiments, the amino acid sequence of the engineered RNA ligase comprises at least a substitution set at amino acid positions 12/230/251, wherein the amino acid positions are relative to the reference sequence corresponding to residues 12 to 346 of SEQ ID NO: 2, or relative to the reference sequence corresponding to SEQ ID NO: 2. In some embodiments, the amino acid sequence of the engineered RNA ligase comprises at least a substitution set 12T/230T/251S, wherein the amino acid positions are relative to the reference sequence corresponding to residues 12 to 346 of SEQ ID NO: 2, or relative to the reference sequence corresponding to SEQ ID NO: 2.

In some embodiments, the amino acid sequence of the engineered RNA ligase comprises at least a substitution at amino acid position 198 or 326, or combination thereof, wherein the amino acid positions are relative to the reference sequence corresponding to residues 12 to 346 of SEQ ID NO: 2, or relative to the reference sequence corresponding to SEQ ID NO: 2. In some embodiments, the amino acid sequence of the engineered RNA ligase comprises at least a substitution or amino acid residue 198M or 326L, or combination thereof, wherein the amino acid positions are relative to the reference sequence corresponding to residues 12 to 346 of SEQ ID NO: 2, or relative to the reference sequence corresponding to SEQ ID NO: 2. In some embodiments, the amino acid sequence of the engineered RNA ligase comprises at least a substitution H198M or K326L, or combination thereof, wherein the amino acid positions are relative to the reference sequence corresponding to residues 12 to 346 of SEQ ID NO: 2, or relative to the reference sequence corresponding to SEQ ID NO: 2. In some embodiments, the amino acid sequence of the engineered RNA ligase comprises at least a substitution set at amino acid positions 198/326, wherein the amino acid positions are relative to the reference sequence corresponding to residues 12 to 346 of SEQ ID NO: 2, or relative to the reference sequence corresponding to SEQ ID NO: 2. In some embodiments, the amino acid sequence of the engineered RNA ligase comprises at least a substitution set 198M/326L, wherein the amino acid positions are relative to the reference sequence corresponding to residues 12 to 346 of SEQ ID NO: 2, or relative to the reference sequence corresponding to SEQ ID NO: 2.

In some embodiments, the amino acid sequence of the engineered RNA ligase comprises at least a substitution at amino acid position 194, 298, or 331, or any combinations thereof, wherein the amino acid positions are relative to the reference sequence corresponding to residues 12 to 346 of SEQ ID NO: 2, or relative to the reference sequence corresponding to SEQ ID NO: 2. In some embodiments, the amino acid sequence of the engineered RNA ligase comprises at least a substitution or amino acid residue 194V, 298S, or 331G, or any combinations thereof, wherein the amino acid positions are relative to the reference sequence corresponding to residues 12 to 346 of SEQ ID NO: 2, or relative to the reference sequence corresponding to SEQ ID NO: 2. In some embodiments, the amino acid sequence of the engineered RNA ligase comprises at least a substitution T194V, M298S, or 1331G, or any combinations thereof, wherein the amino acid positions are relative to the reference sequence corresponding to residues 12 to 346 of SEQ ID NO: 2, or relative to the reference sequence corresponding to SEQ ID NO: 2. In some embodiments, the amino acid sequence of the engineered RNA ligase comprises at least a substitution set at amino acid positions 194/298/331, wherein the amino acid positions are relative to the reference sequence corresponding to residues 12 to 346 of SEQ ID NO: 2, or relative to the reference sequence corresponding to SEQ ID NO: 2. In some embodiments, the amino acid sequence of the engineered RNA ligase comprises at least a substitution set 194V/298S/331G, wherein the amino acid positions are relative to the reference sequence corresponding to residues 12 to 346 of SEQ ID NO: 2, or relative to the reference sequence corresponding to SEQ ID NO: 2.

In some embodiments, the amino acid sequence of the engineered RNA ligase comprises at least a substitution at amino acid position 12 or 25, or combination thereof, wherein the amino acid positions are relative to the reference sequence corresponding to residues 12 to 346 of SEQ ID NO: 2, or relative to the reference sequence corresponding to SEQ ID NO: 2. In some embodiments, the amino acid sequence of the engineered RNA ligase comprises at least a substitution or amino acid residue 12M or 25A, or combination thereof, wherein the amino acid positions are relative to the reference sequence corresponding to residues 12 to 346 of SEQ ID NO: 2, or relative to the reference sequence corresponding to SEQ ID NO: 2. In some embodiments, the amino acid sequence of the engineered RNA ligase comprises at least a substitution T12M or N25A, or combination thereof, wherein the amino acid positions are relative to the reference sequence corresponding to residues 12 to 346 of SEQ ID NO: 2, or relative to the reference sequence corresponding to SEQ ID NO: 2. In some embodiments, the amino acid sequence of the engineered RNA ligase comprises at least a substitution set at amino acid positions 12/25, wherein the amino acid positions are relative to the reference sequence corresponding to residues 12 to 346 of SEQ ID NO: 2, or relative to the reference sequence corresponding to SEQ ID NO: 2. In some embodiments, the amino acid sequence of the engineered RNA ligase comprises at least a substitution set 12M/25A, wherein the amino acid positions are relative to the reference sequence corresponding to residues 12 to 346 of SEQ ID NO: 2, or relative to the reference sequence corresponding to SEQ ID NO: 2.

In some embodiments, the amino acid sequence of the engineered RNA ligase comprises at least a substitution at amino acid position 89, 157, 183, or 214, or any combinations thereof, wherein the amino acid positions are relative to the reference sequence corresponding to residues 12 to 346 of SEQ ID NO: 2, or relative to the reference sequence corresponding to SEQ ID NO: 2. In some embodiments, the amino acid sequence of the engineered RNA ligase comprises at least a substitution or amino acid residue 89M, 157V, 183L, or 214L, or any combinations thereof, wherein the amino acid positions are relative to the reference sequence corresponding to residues 12 to 346 of SEQ ID NO: 2, or relative to the reference sequence corresponding to SEQ ID NO: 2. In some embodiments, the amino acid sequence of the engineered RNA ligase comprises at least a substitution K89M, L157V, 1183L, or V214L, or any combinations thereof, wherein the amino acid positions are relative to the reference sequence corresponding to residues 12 to 346 of SEQ ID NO: 2, or relative to the reference sequence corresponding to SEQ ID NO: 2. In some embodiments, the amino acid sequence of the engineered RNA ligase comprises at least a substitution set at amino acid positions 89/157/183/214, wherein the amino acid positions are relative to the reference sequence corresponding to residues 12 to 346 of SEQ ID NO: 2, or relative to the reference sequence corresponding to SEQ ID NO: 2. In some embodiments, the amino acid sequence of the engineered RNA ligase comprises at least a substitution set 89M/157V/183L/214L, wherein the amino acid positions are relative to the reference sequence corresponding to residues 12 to 346 of SEQ ID NO: 2, or relative to the reference sequence corresponding to SEQ ID NO: 2.

In some embodiments, the amino acid sequence of the engineered RNA ligase comprises at least a substitution at amino acid position 29, 226, or 230, or any combinations thereof, wherein the amino acid positions are relative to the reference sequence corresponding to residues 12 to 346 of SEQ ID NO: 2, or relative to the reference sequence corresponding to SEQ ID NO: 2. In some embodiments, the amino acid sequence of the engineered RNA ligase comprises at least a substitution or amino acid residue 29S, 226R, or 230V, or any combinations thereof, wherein the amino acid positions are relative to the reference sequence corresponding to residues 12 to 346 of SEQ ID NO: 2, or relative to the reference sequence corresponding to SEQ ID NO: 2. In some embodiments, the amino acid sequence of the engineered RNA ligase comprises at least a substitution E29S, I226R, or T230V, or any combinations thereof, wherein the amino acid positions are relative to the reference sequence corresponding to residues 12 to 346 of SEQ ID NO: 2, or relative to the reference sequence corresponding to SEQ ID NO: 2. In some embodiments, the amino acid sequence of the engineered RNA ligase comprises at least a substitution set at amino acid positions 29/226/230, wherein the amino acid positions are relative to the reference sequence corresponding to residues 12 to 346 of SEQ ID NO: 2, or relative to the reference sequence corresponding to SEQ ID NO: 2. In some embodiments, the amino acid sequence of the engineered RNA ligase comprises at least a substitution set 29S/226R/230V, wherein the amino acid positions are relative to the reference sequence corresponding to residues 12 to 346 of SEQ ID NO: 2, or relative to the reference sequence corresponding to SEQ ID NO: 2.

In some embodiments, the amino acid sequence of the engineered RNA ligase comprises at least a substitution at amino acid position 23 or 254, or combination thereof, wherein the amino acid positions are relative to the reference sequence corresponding to residues 12 to 346 of SEQ ID NO: 2, or relative to the reference sequence corresponding to SEQ ID NO: 2. In some embodiments, the amino acid sequence of the engineered RNA ligase comprises at least a substitution or amino acid residue 23T or 254K, or combination thereof, wherein the amino acid positions are relative to the reference sequence corresponding to residues 12 to 346 of SEQ ID NO: 2, or relative to the reference sequence corresponding to SEQ ID NO: 2. In some embodiments, the amino acid sequence of the engineered RNA ligase comprises at least a substitution Y23T or S254K, or combination thereof, wherein the amino acid positions are relative to the reference sequence corresponding to residues 12 to 346 of SEQ ID NO: 2, or relative to the reference sequence corresponding to SEQ ID NO: 2. In some embodiments, the amino acid sequence of the engineered RNA ligase comprises at least a substitution set at amino acid positions 23/254, wherein the amino acid positions are relative to the reference sequence corresponding to residues 12 to 346 of SEQ ID NO: 2, or relative to the reference sequence corresponding to SEQ ID NO: 2. In some embodiments, the amino acid sequence of the engineered RNA ligase comprises at least a substitution set 23T/254K, wherein the amino acid positions are relative to the reference sequence corresponding to residues 12 to 346 of SEQ ID NO: 2, or relative to the reference sequence corresponding to SEQ ID NO: 2.

In some embodiments, the amino acid sequence of the engineered RNA ligase comprises at least a substitution at amino acid position 75, 250, 268, or 307, or an combinations thereof, wherein the amino acid positions are relative to the reference sequence corresponding to residues 12 to 346 of SEQ ID NO: 2, or relative to the reference sequence corresponding to SEQ ID NO: 2. In some embodiments, the amino acid sequence of the engineered RNA ligase comprises at least a substitution or amino acid residue 75D, 250I, 268G, or 307R, or any combinations thereof, wherein the amino acid positions are relative to the reference sequence corresponding to residues 12 to 346 of SEQ ID NO: 2, or relative to the reference sequence corresponding to SEQ ID NO: 2. In some embodiments, the amino acid sequence of the engineered RNA ligase comprises at least a substitution G75D, D250I, E268G, or N307R, or any combinations thereof, wherein the amino acid positions are relative to the reference sequence corresponding to residues 12 to 346 of SEQ ID NO: 2, or relative to the reference sequence corresponding to SEQ ID NO: 2. In some embodiments, the amino acid sequence of the engineered RNA ligase comprises at least a substitution set at amino acid positions 75/250/268/307, wherein the amino acid positions are relative to the reference sequence corresponding to residues 12 to 346 of SEQ ID NO: 2, or relative to the reference sequence corresponding to SEQ ID NO: 2. In some embodiments, the amino acid sequence of the engineered RNA ligase comprises at least a substitution set 75D/250I/268G/307R, wherein the amino acid positions are relative to the reference sequence corresponding to residues 12 to 346 of SEQ ID NO: 2, or relative to the reference sequence corresponding to SEQ ID NO: 2.

In some embodiments, the amino acid sequence of the engineered RNA ligase comprises at least a substitution at amino acid position 38, 93, 192, 248, or 251, or an combinations thereof, wherein the amino acid positions are relative to the reference sequence corresponding to residues 12 to 346 of SEQ ID NO: 2, or relative to the reference sequence corresponding to SEQ ID NO: 2. In some embodiments, the amino acid sequence of the engineered RNA ligase comprises at least a substitution or amino acid residue 38S, 93K, 192E, 248F, or 251E, or any combinations thereof, wherein the amino acid positions are relative to the reference sequence corresponding to residues 12 to 346 of SEQ ID NO: 2, or relative to the reference sequence corresponding to SEQ ID NO: 2. In some embodiments, the amino acid sequence of the engineered RNA ligase comprises at least a substitution M38S, V93K, D192E, K248F, or S251E, or any combinations thereof, wherein the amino acid positions are relative to the reference sequence corresponding to residues 12 to 346 of SEQ ID NO: 2, or relative to the reference sequence corresponding to SEQ ID NO: 2. In some embodiments, the amino acid sequence of the engineered RNA ligase comprises at least a substitution set at amino acid positions 38/93/192/248/251, wherein the amino acid positions are relative to the reference sequence corresponding to residues 12 to 346 of SEQ ID NO: 2, or relative to the reference sequence corresponding to SEQ ID NO: 2. In some embodiments, the amino acid sequence of the engineered RNA ligase comprises at least a substitution set 38S/93K/192E/248F/251E, wherein the amino acid positions are relative to the reference sequence corresponding to residues 12 to 346 of SEQ ID NO: 2, or relative to the reference sequence corresponding to SEQ ID NO: 2.

In some embodiments, the amino acid sequence of the engineered RNA ligase comprises at least a substitution at amino acid position 38, 86, 101, 241, or 318, or an combinations thereof, wherein the amino acid positions are relative to the reference sequence corresponding to residues 12 to 346 of SEQ ID NO: 2, or relative to the reference sequence corresponding to SEQ ID NO: 2. In some embodiments, the amino acid sequence of the engineered RNA ligase comprises at least a substitution or amino acid residue 38A, 86D, 101P, 241G, or 318S, or any combinations thereof, wherein the amino acid positions are relative to the reference sequence corresponding to residues 12 to 346 of SEQ ID NO: 2, or relative to the reference sequence corresponding to SEQ ID NO: 2. In some embodiments, the amino acid sequence of the engineered RNA ligase comprises at least a substitution M38A, K86D, A101P, S241G, or F318S, or any combinations thereof, wherein the amino acid positions are relative to the reference sequence corresponding to residues 12 to 346 of SEQ ID NO: 2, or relative to the reference sequence corresponding to SEQ ID NO: 2. In some embodiments, the amino acid sequence of the engineered RNA ligase comprises at least a substitution set at amino acid positions 38/86/101/241/318, wherein the amino acid positions are relative to the reference sequence corresponding to residues 12 to 346 of SEQ ID NO: 2, or relative to the reference sequence corresponding to SEQ ID NO: 2. In some embodiments, the amino acid sequence of the engineered RNA ligase comprises at least a substitution set 38A/86D/101P/241G/318S, wherein the amino acid positions are relative to the reference sequence corresponding to residues 12 to 346 of SEQ ID NO: 2, or relative to the reference sequence corresponding to SEQ ID NO: 2.

In some embodiments, the amino acid sequence of the engineered RNA ligase comprises at least a substitution at amino acid position 122, 143, 212, or 295, or an combinations thereof, wherein the amino acid positions are relative to the reference sequence corresponding to residues 12 to 346 of SEQ ID NO: 2, or relative to the reference sequence corresponding to SEQ ID NO: 2. In some embodiments, the amino acid sequence of the engineered RNA ligase comprises at least a substitution or amino acid residue 122F, 143S, 212E, or 295V, or any combinations thereof, wherein the amino acid positions are relative to the reference sequence corresponding to residues 12 to 346 of SEQ ID NO: 2, or relative to the reference sequence corresponding to SEQ ID NO: 2. In some embodiments, the amino acid sequence of the engineered RNA ligase comprises at least a substitution V122F, V143S, T212E, or G295V, or any combinations thereof, wherein the amino acid positions are relative to the reference sequence corresponding to residues 12 to 346 of SEQ ID NO: 2, or relative to the reference sequence corresponding to SEQ ID NO: 2. In some embodiments, the amino acid sequence of the engineered RNA ligase comprises at least a substitution set at amino acid positions 122/143/212/295, wherein the amino acid positions are relative to the reference sequence corresponding to residues 12 to 346 of SEQ ID NO: 2, or relative to the reference sequence corresponding to SEQ ID NO: 2. In some embodiments, the amino acid sequence of the engineered RNA ligase comprises at least a substitution set 122F/143S/212E/295V, wherein the amino acid positions are relative to the reference sequence corresponding to residues 12 to 346 of SEQ ID NO: 2, or relative to the reference sequence corresponding to SEQ ID NO: 2.

In some embodiments, the amino acid sequence of the engineered RNA ligase comprises at least a substitution at amino acid position 291, 295, or 298, or an combinations thereof, wherein the amino acid positions are relative to the reference sequence corresponding to residues 12 to 346 of SEQ ID NO: 2, or relative to the reference sequence corresponding to SEQ ID NO: 2. In some embodiments, the amino acid sequence of the engineered RNA ligase comprises at least a substitution or amino acid residue 291Q, 295G, or 298V, or any combinations thereof, wherein the amino acid positions are relative to the reference sequence corresponding to residues 12 to 346 of SEQ ID NO: 2, or relative to the reference sequence corresponding to SEQ ID NO: 2. In some embodiments, the amino acid sequence of the engineered RNA ligase comprises at least a substitution A291Q, V295G, or S298V, or any combinations thereof, wherein the amino acid positions are relative to the reference sequence corresponding to residues 12 to 346 of SEQ ID NO: 2, or relative to the reference sequence corresponding to SEQ ID NO: 2. In some embodiments, the amino acid sequence of the engineered RNA ligase comprises at least a substitution set at amino acid positions 291/295/298, wherein the amino acid positions are relative to the reference sequence corresponding to residues 12 to 346 of SEQ ID NO: 2, or relative to the reference sequence corresponding to SEQ ID NO: 2. In some embodiments, the amino acid sequence of the engineered RNA ligase comprises at least a substitution set 291Q/295G/298V, wherein the amino acid positions are relative to the reference sequence corresponding to residues 12 to 346 of SEQ ID NO: 2, or relative to the reference sequence corresponding to SEQ ID NO: 2.

In some embodiments, the amino acid sequence of the engineered RNA ligase further comprises at least a substitution at amino acid position 2, 14, 15, 63, 69, 81, 85, 95, 138, 142, 149, 159, 171, 186, 195, 202, 266, 275, 283, 285, 303, 314, 333, or 339; a substitution or amino acid residue 181, 33L, 39Q/S, 86G, 89G/H/I/M/W, 116N, 117D, 119T, 144W, 154C, 156A/L/N/Q/T, 175K, 177P, 181D, 183V, 185G/K, 212I, 2141, 224Y, 2261, 230D, 247K, 256A, 257L, 260S, 280G/N, 288E, 291P, 296K, 306L, 307E/Q, 310K, 315S/T, 316L, 317A, 326R, 330M/R, 331L/R/W, 334R, 335D/H, 337L, 3421, or 345V; or any combinations thereof, wherein the amino acid positions are relative to the reference sequence corresponding to residues 12 to 346 of SEQ ID NO: 2, or relative to the reference sequence corresponding to SEQ ID NO: 2.

In some embodiments, the amino acid sequence of the engineered RNA ligase further comprises at least a substitution or amino acid residue 2E/G/V, 14K, 15E/L/M/P/Y, 18I, 33L, 39Q/S, 63V, 69D, 81Y, 85L, 86G, 89G/H/I/M/W, 95V, 116N, 117D, 119T, 138R, 142R, 144W, 149R/T, 154C, 156A/L/N/Q/T, 159F, 171E, 175K, 177P, 181D, 183V, 185G/K, 186D/R, 195G, 202Y, 2121, 2141, 224Y, 2261, 230D, 247K, 256A, 257L, 260S, 266L, 275N, 280G/N, 2831, 285K, 288E, 291P, 296K, 303Q, 306L, 307E/Q, 310K, 314W, 315S/T, 316L, 317A, 326R, 330M/R, 331L/R/W, 333V, 334R, 335D/H, 337L, 339P, 342I, or 345V, or any combinations thereof, wherein the amino acid positions are relative to the reference sequence corresponding to residues 12 to 346 of SEQ ID NO: 2, or relative to the reference sequence corresponding to SEQ ID NO: 2.

In some embodiments, the amino acid sequence of the engineered RNA ligase comprises at least a substitution or substitution set at amino acid position(s) 256, 258, 254, 251, 248, 37, 54, 232, 15, 71, 26, 116, 38, 25, 106/251, 52, 33, 247, 37/249/266/316/330/334/335, 21, 11/37/249/266/316, 261, 11/266/316, 11/248/249/266, 331, 11/248/249, 255, 37/249/266/316/334, 266, 11/37/156/249/335, 37/156/248/249/266/334, 334, 156, 11/266/316/334, 11/39/156/248/316, 11/249, 37/156,107/251, 156/249/316, 37/156/316, 11/37/156/249, 11/37/334, 194, 37/316,175, 11/156/249/316, 11/37, 252, 12, 37/266, 11/37/156/266, 11/37/156/248/266, 37/236/248/249, 37/156/248/249, 11/266/330/335, 249, 11/156/248/249/316, 37/249/316, 11, 11/156, 11/37/249, 330, 316, 156/249/266/335, 249/266/316/330/335, 11/156/249, 266/316/330/334/335, 156/248/249/316/334, 37/156/248/249/316, 156/266, 248/249, 156/249/316/330/335, 218, 37/156/248/249/266, 11/37/156/248/249/316, 37/156/316/330, 11/37/248/249/316/330, 11/37/248/249, 249/330/335, 11/37/156/330/334, 40, 11/37/148/249/316, 83, 323, 193, 335, or 257, wherein the amino acid positions are relative to the reference sequence corresponding to residues 12 to 346 of SEQ ID NO: 2, or relative to the reference sequence corresponding to SEQ ID NO: 2.

In some embodiments, the amino acid sequence of the engineered RNA ligase comprises at least a substitution or substitution set, or amino acid residue(s) 256A, 258P, 254L, 251S, 248T, 37M, 54L, 258M, 232S, 15M, 71T, 26L, 232M, 116L, 38S, 251L, 25S, 106G/251F, 52C, 33L, 251G, 256S, 247L, 37L/249R/266L/316L/330R/334R/335H, 21S, 11N/37L/249L/266L/316L, 261P, 11N/266L/316L, 11N/248T/249R/266L, 331D, 11N/248T/249L, 37F, 255V, 37L/249R/266L/316L/334R, 38L, 266L, 232R, 11N/37L/156Q/249R/335H, 37L/156Q/248T/249R/266L/334R, 334E, 156Q, 11N/266L/316L/334R, 11N/39P/156Q/248T/316L, 11N/249R, 37L/156Q, 107M/251R, 156Q/249R/316L, 37L/156Q/316L, 11N/37L/156Q/249L, 11N/37L/334R, 194A, 37L/316L, 175A, 11N/156Q/249L/316L, 11N/37L, 252Q, 37L, 12F, 11N/249L, 37L/266L, 11N/37L/156Q/266L, 11N/37L/156Q/248T/266L, 37L/236L/248T/249R, 37L/156Q/248T/249L, 11N/266L/330R/335H, 249L, 11N/156Q/248T/249R/316L, 37L/249L/316L, 11N, 11N/156Q, 11N/37L/249L, 330R, 316L, 156Q/249L/266L/335H, 249L/266L/316L/330R/335H, 11N/156Q/249L, 249R, 266L/316L/330R/334R/335H, 254V, 156Q/248T/249L/316L/334R, 33A, 37L/156Q/248T/249R/316L, 156Q/266L, 248T/249L, 25M, 156Q/249R/316L/330R/335H, 218F, 12V, 12T, 37L/156Q/248T/249L/266L, 12L, 11N/37L/156Q/248T/249L/316L, 37L/156Q/316L/330R, 11N/37L/248T/249R/316L/330R, 11N/37L/248T/249L, 249R/330R/335H, 11N/37L/156Q/330R/334R, 25L, 38R, 40D, 248T/249R, 11N/37L/148F/249L/316L, 83V, 323E, 193Q, 335H, 257G, or 258V, wherein the amino acid positions are relative to the reference sequence corresponding to residues 12 to 346 of SEQ ID NO: 2, or relative to the reference sequence corresponding to SEQ ID NO: 2.

In some embodiments, the amino acid sequence of the engineered RNA ligase comprises at least a substitution or substitution set or amino acid residue(s) K256A, E258P, K254L, K251S, K248T, D37M, F54L, E258M, T232S, Y15M, P71T, K26L, T232M, G116L, M38S, K251L, N25S, S106G/K251F, T52C, G33L, K251G, K256S, A247L, D37L/S249R/V266L/M316L/T330R/Q334R/N335H, N21S, G11N/D37L/S249L/V266L/M316L, E261P, G11N/V266L/M316L, G11N/K248T/S249R/V266L, 1331D, G11N/K248T/S249L, D37F, A255V, D37L/S249R/V266L/M316L/Q334R, M38L, V266L, T232R, G11N/D37L/T156Q/S249R/N335H, D37L/T156Q/K248T/S249R/V266L/Q334R, Q334E, T156Q, G11N/V266L/M316L/Q334R, G11N/H39P/T156Q/K248T/M316L, G11N/S249R, D37L/T156Q, V107M/K251R, T156Q/S249R/M316L, D37L/T156Q/M316L, G11N/D37L/T156Q/S249L, G11N/D37L/Q334R, T194A, D37L/M316L, Q175A, G11N/T156Q/S249L/M316L, G11N/D37L, P252Q, D37L, M12F, G11N/S249L, D37L/V266L,G11N/D37L/T156Q/V266L, G11N/D37L/T156Q/K248T/V266L, D37L/1236L/K248T/S249R, D37L/T156Q/K248T/S249L, G11N/V266L/T330R/N335H, S249L, G11N/T156Q/K248T/S249R/M316L, D37L/S249L/M316L, G11N, G11N/T156Q, G11N/D37L/S249L, T330R, M316L, T156Q/S249L/V266L/N335H, S249L/V266L/M316L/T330R/N335H, G11N/T156Q/S249L, S249R, V266L/M316L/T330R/Q334R/N335H, K254V, T156Q/K248T/S249L/M316L/Q334R, G33A, D37L/T156Q/K248T/S249R/M316L, T156Q/V266L, K248T/S249L, N25M, T156Q/S249R/M316L/T330R/N335H, Y218F, M12V, M12T, D37L/T156Q/K248T/S249L/V266L, M12L, G11N/D37L/T156Q/K248T/S249L/M316L, D37L/T156Q/M316L/T330R, G11N/D37L/K248T/S249R/M316L/T330R, G11N/D37L/K248T/S249L, S249R/T330R/N335H, G11N/D37L/T156Q/T330R/Q334R, N25L, M38R, T40D, K248T/S249R, G11N/D37L/Y148F/S249L/M316L, 183V, S323E, T193Q, N335H, V257G, or E258V, wherein the amino acid positions are relative to the reference sequence corresponding to residues 12 to 346 of SEQ ID NO: 2, or relative to the reference sequence corresponding to SEQ ID NO: 2.

In some embodiments, the amino acid sequence of the engineered RNA ligase comprises at least a substitution set at amino acid positions 11/37/156/232/249/266/316/334/335, 11/12/37/249/254/256/266/316, 11/12/37/230/249/266/316, 11/12/37/230/249/251/266/316, 11/12/37/230/249/251/254/256/266/316, 11/12/37/38/230/249/266/316, 11/37/230/249/251/266/316, 11/12/37/38/249/254/266/316, 11/37/230/249/256/266/316, 11/37/249/254/266/316, 11/37/230/249/266/316, 11/12/37/230/249/254/266/316, 11/12/37/38/230/249/251/256/266/316, 11/12/37/249/251/266/316, 11/12/37/249/251/254/256/266/316, 11/37/38/249/266/316, 11/12/37/230/249/256/266/316, 11/12/37/249/266/316, 11/12/25/37/38/230/249/251/266/316, 11/12/37/38/102/230/249/251/256/266/316, 11/12/37/249/256/266/316, 11/37/38/230/249/256/266/316, 11/37/249/256/266/316, 11/12/37/230/249/251/256/266/316, 11/37/230/249/251/254/266/316, 11/37/230/249/254/266/316, 11/37/249/251/254/266/316, 11/12/25/37/230/249/266/316, 11/37/38/230/249/266/316, 11/37/38/249/251/266/316, 11/12/37/38/230/249/256/266/316, 11/37/102/249/266/316, 11/37/249/251/266/316, 11/12/25/37/249/251/256/266/316, 11/12/37/102/249/256/266/316, 11/12/37/38/230/249/251/254/256/266/316, 11/12/37/249/251/256/266/316, 11/37/102/230/249/251/254/256/266/316, 11/12/37/38/249/251/266/316, 11/12/37/230/249/251/254/266/316, 11/37/156/248/249/266/316/334/335, 11/12/25/37/38/249/266/316, 11/12/37/102/230/249/251/254/266/316, 11/12/37/38/249/251/254/266/316, 11/12/37/249/251/254/266/316, 11/12/37/102/249/266/316, 11/37/38/230/249/251/254/256/266/316, 11/12/25/37/230/249/251/256/266/316, 11/12/37/230/249/266/311/316, 11/12/37/249/254/266/316, 11/37/38/230/249/251/254/266/316, 11/12/37/38/249/251/256/266/316, 11/37/156/249/266/316/334/335, 11/37/249/257/258/266/316/334, 11/12/25/37/38/249/251/256/266/316, 11/37/249/266/316/334, 11/12/25/37/230/249/254/256/266/316, 11/37/38/249/251/256/266/316, 11/12/37/38/230/249/251/254/266/316, 11/12/37/38/249/266/316, 11/37/249/257/258/266/316/334/335, 11/12/37/38/249/254/256/266/316, 11/37/232/248/249/257/258/266/316/334/335, 11/37/156/249/258/266/316/334/335, 11/12/37/102/230/249/254/266/316, 11/25/37/249/266/316, 11/37/156/249/257/258/266/316/334/335, 11/12/37/102/230/249/266/316, 11/37/156/248/249/257/258/266/316/334, 11/37/248/249/266/316/334/335, 11/37/232/249/257/258/266/316/334/335, 11/37/249/266/316/334/335, 11/37/156/249/266/316/334, 11/12/37/38/102/249/256/266/316, 11/37/156/249/257/266/316/334/335, 11/12/37/140/249/251/254/266/316, 11/37/249/251/254/256/266/316, 11/37/156/249/258/266/316/334, 11/37/156/248/249/266/316/330/335, 11/12/37/38/249/256/266/316, 11/37/232/249/266/316/334/335, 11/37/249/266/316/330/334/335, 11/37/156/248/249/266/316/334, 11/37/156/249/266/316/335, 11/37/249/257/266/316/334, 11/37/248/249/266/316/334, 11/37/232/248/249/266/316/334/335, 11/12/25/37/38/230/249/251/256/266/316, 11/37/249/266/316/330/335, 11/37/248/249/257/266/316/334/335, 11/37/232/248/249/266/316/334, 11/37/156/232/248/249/266/316/334/335, 11/37/232/249/257/266/316/330/334, 11/37/156/248/249/266/291/316/334/335, 11/37/156/232/248/249/257/258/266/316/334, 11/12/25/37/249/266/316, 11/37/156/248/249/266/316/330/334/335, 11/37/232/248/249/266/316/330/335, 11/37/156/232/249/266/316/330/334/335, 11/37/197/249/266/316, 11/37/249/266/316/338, 11/37/249/266/316/327, 11/37/192/249/266/316, 11/37/249/266/316/331, 11/37/249/266/316/326, 11/37/249/266/316/345, 11/37/101/249/266/316, 11/37/100/249/266/316, 11/37/141/249/266/316, 11/37/198/249/266/316, 11/37/64/249/266/316, 11/37/249/266/306/316, 11/37/249/266/316/317, 11/37/249/265/266/316, 11/37/249/266/296/316, 11/37/194/249/266/316, 11/37/62/249/266/316, 11/37/90/249/266/316, 11/37/249/266/307/316, 11/37/103/249/266/316, 11/37/210/249/266/316, 11/37/105/249/266/316, or 11/37/61/249/266/316, wherein the amino acid positions are relative to the reference sequence corresponding to residues 12 to 346 of SEQ ID NO: 2, or relative to the reference sequence corresponding to SEQ ID NO: 2.

In some embodiments, the amino acid sequence of the engineered RNA ligase comprises at least a substitution set or amino acid residues 11N/37L/156Q/232G/249L/266L/316L/334R/335H, 11N/12V/37L/249L/254V/256A/266L/316L, 11N/12V/37L/230T/249L/266L/316L, 11N/12L/37L/230T/249L/251T/266L/316L, 11N/12L/37L/230T/249L/251S/254V/256A/266L/316L, 11N/12A/37L/38R/230T/249L/266L/316L, 11N/37L/230T/249L/251S/266L/316L, 11N/12P/37L/38R/249L/254V/266L/316L, 11N/12V/37L/230T/249L/251T/266L/316L, 11N/37L/230T/249L/256A/266L/316L, 11N/37L/249L/254V/266L/316L, 11N/37L/230T/249L/266L/316L, 11N/12T/37L/230T/249L/251S/266L/316L, 11N/12T/37L/230T/249L/254V/266L/316L, 11N/12V/37L/38R/230T/249L/251T/256A/266L/316L, 11N/12L/37L/249L/251S/266L/316L, 11N/12T/37L/249L/251T/254V/256A/266L/316L, 11N/12V/37L/230T/249L/251S/266L/316L, 11N/37L/38R/249L/266L/316L, 11N/12T/37L/230T/249L/256A/266L/316L, 11N/12L/37L/249L/266L/316L, 11N/12V/37L/249L/266L/316L, 11N/12L/25M/37L/38R/230T/249L/251T/266L/316L, 11N/12L/37L/38R/102T/230T/249L/251T/256A/266L/316L, 11N/12L/37L/230T/249L/266L/316L, 11N/12V/37L/249L/256A/266L/316L, 11N/37L/38R/230T/249L/256A/266L/316L, 11N/12V/37L/230T/249L/251S/254V/256A/266L/316L, 11N/37L/249L/256A/266L/316L, 11N/12T/37L/230T/249L/251S/256A/266L/316L, 11N/12T/37L/249L/251T/266L/316L, 11N/37L/230T/249L/251S/254L/266L/316L, 11N/37L/230T/249L/254V/266L/316L, 11N/37L/249L/251S/254L/266L/316L, 11N/12T/25L/37L/230T/249L/266L/316L, 11N/12V/37L/230T/249L/251S/254L/256A/266L/316L, 11N/37L/38R/230T/249L/266L/316L, 11N/12T/37L/249L/251S/266L/316L, 11N/37L/38R/249L/251T/266L/316L, 11N/12V/37L/230T/249L/251S/256A/266L/316L, 11N/12L/37L/230T/249L/251T/254V/256A/266L/316L, 11N/12T/37L/38R/230T/249L/256A/266L/316L, 11N/12L/37L/230T/249L/256A/266L/316L, 11N/37L/102T/249L/266L/316L, 11N/37L/249L/251T/266L/316L, 11N/12V/25M/37L/249L/251T/256A/266L/316L, 11N/12T/37L/102T/249L/256A/266L/316L, 11N/37L/230T/249L/251T/266L/316L, 11N/12T/37L/230T/249L/251S/254L/256A/266L/316L, 11N/12V/37L/38R/230T/249L/251T/254V/256A/266L/316L, 11N/12T/37L/38R/230T/249L/266L/316L, 11N/12L/37L/38R/249L/254V/266L/316L, 11N/12L/37L/249L/251T/256A/266L/316L, 11N/12T/37L/230T/249L/266L/316L, 11N/37L/102T/230T/249L/251S/254V/256A/266L/316L, 11N/37L/230T/249L/251S/254V/266L/316L, 11N/12T/37L/230T/249L/251S/254V/256A/266L/316L, 11N/12T/37L/38R/249L/251S/266L/316L, 11N/12T/37L/230T/249L/251S/254V/266L/316L, 11N/12T/37L/230T/249L/251T/266L/316L, 11N/37L/156Q/248T/249L/266L/316L/334R/335H, 11N/12V/37L/38R/230T/249L/266L/316L, 11N/12T/25M/37L/38R/249L/266L/316L, 11N/12V/37L/102T/230T/249L/251T/254V/266L/316L, 11N/12L/37L/249L/256A/266L/316L, 11N/12T/37L/249L/266L/316L, 11N/37L/230T/249L/251T/254L/266L/316L, 11N/12V/25L/37L/38R/249L/266L/316L, 11N/12T/37L/38R/249L/251S/254V/266L/316L, 11N/12V/37L/249L/251S/254L/256A/266L/316L, 11N/12T/37L/249L/251T/254V/266L/316L, 11N/12T/37L/102T/249L/266L/316L, 11N/12V/37L/38R/249L/251S/266L/316L, 11N/12V/37L/230T/249L/251S/254L/266L/316L, 11N/12T/37L/249L/251T/256A/266L/316L, 11N/37L/38R/230T/249L/251S/254V/256A/266L/316L, 11N/12V/37L/230T/249L/251T/256A/266L/316L, 11N/12L/25M/37L/230T/249L/251S/256A/266L/316L, 11N/12T/37L/230T/249L/266L/311C/316L, 11N/12L/37L/38R/230T/249L/251S/254V/256A/266L/316L, 11N/12L/37L/230T/249L/251T/254L/266L/316L, 11N/12L/37L/249L/254V/266L/316L, 11N/37L/38R/230T/249L/251S/254V/266L/316L, 11N/12L/37L/38R/249L/251S/256A/266L/316L, 11N/12L/37L/38R/249L/251S/266L/316L, 11N/37L/156Q/249L/266L/316L/334R/335H, 11N/12A/37L/230T/249L/266L/316L, 11N/12P/37L/230T/249L/266L/316L, 11N/12T/37L/38R/249L/251T/266L/316L, 11N/37L/249L/257G/258P/266L/316L/334R, 11N/12L/25L/37L/38R/249L/251T/256A/266L/316L, 11N/37L/249L/266L/316L/334R, 11N/12T/25L/37L/230T/249L/254V/256A/266L/316L, 11N/12T/37L/249L/251S/256A/266L/316L, 11N/37L/38R/249L/251T/256A/266L/316L, 11N/12V/37L/249L/251S/266L/316L, 11N/12V/37L/249L/251T/254L/266L/316L, 11N/12V/37L/38R/230T/249L/251T/254V/266L/316L, 11N/12L/37L/38R/249L/266L/316L, 11N/12L/37L/249L/251S/254L/256A/266L/316L, 11N/37L/249L/257G/258P/266L/316L/334R/335H, 11N/12L/37L/38R/249L/254L/256A/266L/316L, 11N/12V/37L/249L/254L/266L/316L, 11N/12T/37L/249L/254L/266L/316L, 11N/12T/37L/38R/249L/266L/316L, 11N/37L/232G/248T/249L/257G/258P/266L/316L/334R/335H, 11N/12L/37L/102T/249L/266L/316L, 11N/37L/156Q/249L/258P/266L/316L/334R/335H, 11N/37L/156Q/249R/266L/316L/334R/335H, 11N/12T/37L/102T/230T/249L/254L/266L/316L, 11N/25M/37L/249L/266L/316L, 11N/12V/37L/249L/254L/256A/266L/316L, 11N/37L/156H/249L/266L/316L/334R/335H, 11N/37L/156Q/249L/257G/258P/266L/316L/334R/335H, 11N/12T/37L/102T/230T/249L/266L/316L, 11N/37L/156Q/248T/249L/257G/258P/266L/316L/334R, 11N/12T/37L/249L/256A/266L/316L, 11N/37L/248T/249R/266L/316L/334R/335H, 11N/37L/232G/249L/257G/258P/266L/316L/334R/335H, 11N/12T/37L/38R/249L/251T/254V/266L/316L, 11N/37L/249L/266L/316L/334R/335H, 11N/37L/156Q/249L/266L/316L/334R, 11N/12V/37L/38R/249L/254V/266L/316L, 11N/37L/249L/251T/254V/266L/316L, 11N/12T/37L/38R/102T/249L/256A/266L/316L, 11N/37L/156Q/249L/257G/266L/316L/334R/335H, 11N/37L/38R/249L/251S/256A/266L/316L, 11N/12T/37L/140I/249L/251S/254L/266L/316L, 11N/37L/249L/251S/254V/256A/266L/316L, 11N/12V/37L/249L/251T/266L/316L, 11N/37L/248T/249L/266L/316L/334R/335H, 11N/12T/37L/249L/254V/266L/316L, 11N/12V/37L/38R/249L/266L/316L, 11N/12V/37L/249L/251T/256A/266L/316L, 11N/37L/156Q/249L/258P/266L/316L/334R, 11N/37L/156Q/248T/249L/266L/316L/330R/335H, 11N/12T/37L/38R/249L/256A/266L/316L, 11N/37L/232G/249L/266L/316L/334R/335H, 11N/37L/156Q/249R/266L/316L/334R, 11N/37L/249L/266L/316L/330R/334R/335H, 11N/37L/156Q/248T/249L/266L/316L/334R, 11N/37L/156Q/249L/266L/316L/335H, 11N/37L/249L/257G/266L/316L/334R, 11N/37L/156H/248T/249L/266L/316L/334R/335H, 11N/37L/248T/249L/266L/316L/334R, 11N/37L/232G/248T/249L/266L/316L/334R/335H, 11N/37L/156H/248T/249L/266L/316L/334R, 11N/37L/156H/249L/266L/316L/334R, 11N/12T/25M/37L/38R/230T/249L/251S/256A/266L/316L, 11N/12V/37L/249L/251S/254V/256A/266L/316L, 11N/37L/156Q/248T/249R/266L/316L/334R/335H, 11N/37L/249L/266L/316L/330R/335H, 11N/37L/248T/249L/257G/266L/316L/334R/335H, 11N/37L/232G/248T/249L/266L/316L/334R, 11N/37L/156Q/232G/248T/249L/266L/316L/334R/335H, 11N/37L/232G/249L/257G/266L/316L/330R/334R, 11N/37L/156Q/248T/249L/266L/291T/316L/334R/335H, 11N/37L/156Q/232G/248T/249L/257G/258P/266L/316L/334R, 11N/12V/37L/38R/249L/251T/254L/266L/316L, 11N/12T/25L/37L/38R/249L/266L/316L, 11N/37L/156Q/232G/249R/266L/316L/334R/335H, 11N/12T/25M/37L/249L/266L/316L, 11N/37L/156Q/248T/249L/266L/316L/330R/334R/335H, 11N/37L/156Q/248T/249R/266L/316L/334R, 11N/37L/156H/232G/248T/249L/266L/316L/334R/335H, 11N/37L/232G/248T/249L/266L/316L/330R/335H, 11N/37L/156Q/232G/249L/266L/316L/330R/334R/335H, 11N/37L/197W/249L/266L/316L, 11N/37L/249L/266L/316L/338L, 11N/37L/249L/266L/316L/327S, 11N/37L/192F/249L/266L/316L, 11N/37L/249L/266L/316L/331S, 11N/37L/249L/266L/316L/326C, 11N/37L/249L/266L/316L/338G, 11N/37L/249L/266L/316L/345S, 11N/37L/249L/266L/316L/327G, 11N/37L/101V/249L/266L/316L, 11N/37L/100V/249L/266L/316L, 11N/37L/249L/266L/316L/331G, 11N/37L/141G/249L/266L/316L, 11N/37L/198M/249L/266L/316L, 11N/37L/249L/266L/316L/326L, 11N/37L/64W/249L/266L/316L, 11N/37L/249L/266L/306G/316L, 11N/37L/249L/266L/316L/345Q, 11N/37L/101W/249L/266L/316L, 11N/37L/100R/249L/266L/316L, 11N/37L/249L/266L/316L/331W, 11N/37L/249L/266L/316L/317F, 11N/37L/249L/266L/316L/338M, 11N/37L/249L/265F/266L/316L, 11N/37L/249L/266L/296W/316L, 11N/37L/194V/249L/266L/316L, 11N/37L/62V/249L/266L/316L, 11N/37L/249L/266L/316L/317W, 11N/37L/90F/249L/266L/316L, 11N/37L/249L/266L/307R/316L, 11N/37L/249L/266L/316L/317G, 11N/37L/100T/249L/266L/316L, 11N/37L/100L/249L/266L/316L, 11N/37L/194G/249L/266L/316L, 11N/37L/103G/249L/266L/316L, 11N/37L/62A/249L/266L/316L, 11N/37L/210G/249L/266L/316L, 11N/37L/105R/249L/266L/316L, or 11N/37L/61A/249L/266L/316L, wherein the amino acid positions are relative to the reference sequence corresponding to residues 12 to 346 of SEQ ID NO: 2, or relative to the reference sequence corresponding to SEQ ID NO: 2.

In some embodiments, the amino acid sequence of the engineered RNA ligase comprises at least a substitution set at amino acid positions 11/12/37/141/197/230/249/251/266/316/326/327, 11/12/37/100/101/230/249/251/266/316/338, 11/12/37/141/198/230/249/251/266/316/326, 11/12/37/64/100/101/230/249/251/266/316, 11/12/37/100/230/249/251/266/316/338, 11/12/37/101/141/198/230/249/251/266/316/326, 11/12/37/101/198/230/249/251/266/316/326/327, 11/12/37/198/230/249/251/266/316/326, 11/12/37/64/100/101/230/249/251/266/316/338/345, 11/12/37/197/230/249/251/266/316/326, 11/12/37/198/230/249/251/266/316/327, 11/12/37/141/198/230/249/251/266/316/327, 11/12/37/198/230/249/251/266/316/326/327, 11/12/37/197/230/249/251/266/316/326/327, 11/12/37/64/100/230/249/251/266/316/338, 11/12/37/64/100/101/156/197/230/249/251/266/316/338, 11/12/37/64/100/101/230/249/251/266/316/338, 11/12/37/100/197/230/249/251/266/316/338, 11/12/37/197/230/249/251/266/316/338, 11/12/37/101/197/230/249/251/266/316/326, 11/12/37/64/100/197/230/249/251/266/316/338, 11/12/37/101/198/230/249/251/266/316, 11/12/37/141/197/230/249/251/266/316/327, 11/12/37/64/100/101/197/230/249/251/266/316/338, 11/12/37/101/230/249/251/266/316/326, 11/12/37/101/197/230/249/251/266/316/326/327, 11/12/37/197/230/249/251/266/316/327, 11/12/37/197/230/249/251/266/316, 11/12/37/141/197/230/249/251/266/316, 11/12/37/101/230/249/251/266/316, 11/12/37/101/198/230/249/251/266/316/326, 11/12/37/64/197/198/230/249/251/266/316/338, 11/12/37/141/230/249/251/266/316, 11/12/37/101/141/230/249/251/266/316, 11/12/37/100/101/197/230/249/251/266/316/338, 11/12/37/141/230/249/251/266/316/326, 11/12/37/230/249/251/266/316/338, 11/12/37/230/249/251/266/316/326, 11/12/37/197/198/230/249/251/266/316/338, 11/12/37/64/101/230/249/251/266/316/338, 11/12/37/64/197/198/230/249/251/266/316, 11/12/37/101/197/230/249/251/266/316, 11/12/37/100/101/230/249/251/266/316, 11/12/37/100/109/197/230/249/251/266/316/338, 11/12/37/101/197/230/249/251/266/316/338, 11/12/37/197/198/230/249/251/266/316, 11/12/37/141/230/249/251/266/316/326/327, 11/12/37/100/197/230/249/251/266/316, 11/12/37/64/100/230/249/251/266/316, 11/12/37/64/197/230/249/251/266/316/338, 11/12/37/100/197/198/230/249/251/266/316, 11/12/37/101/230/249/251/266/316/338, 11/12/37/141/230/249/251/266/316/327, 11/12/37/101/230/249/251/266/306/316, 11/12/37/64/100/101/197/230/249/251/266/316, 11/12/37/101/230/249/251/266/316/326/327, 11/12/37/100/230/249/251/266/316, 11/12/37/230/249/251/266/316/327, 11/12/37/230/249/251/266/316/326/345, 11/12/37/64/100/101/197/198/230/249/251/266/316, 11/12/37/230/249/251/266/316/326/327, 11/12/37/64/101/230/249/251/266/316, 11/12/37/100/101/197/230/249/251/266/316, 11/12/37/101/230/249/251/266/316/327, 11/12/37/64/100/197/198/230/249/251/266/316/338, 11/12/37/230/249/251/266/306/316, 11/12/37/64/230/249/251/266/316/338, 11/12/37/101/141/230/249/251/266/316/326/327, 11/12/37/64/101/197/230/249/251/266/316/338, or 11/12/37/101/198/230/249/251/266/306/316/327, wherein the amino acid positions are relative to the reference sequence corresponding to residues 12 to 346 of SEQ ID NO: 2, or relative to the reference sequence corresponding to SEQ ID NO: 2.

In some embodiments, the amino acid sequence of the engineered RNA ligase comprises at least a substitution set or amino acid residues 11N/12T/37L/141G/197W/230T/249L/251S/266L/316L/326L/327S, 11N/12T/37L/100V/101V/230T/249L/251S/266L/316L/338L, 11N/12T/37L/141G/198M/230T/249L/251S/266L/316L/326L, 11N/12T/37L/64W/100V/101V/230T/249L/251S/266L/316L, 11N/12T/37L/100V/230T/249L/251S/266L/316L/338L, 11N/12T/37L/101V/141G/198M/230T/249L/251S/266L/316L/326L, 11N/12T/37L/101V/198M/230T/249L/251S/266L/316L/326L/327G, 11N/12T/37L/100V/101V/230T/249L/251S/266L/316L/338G, 11N/12T/37L/198M/230T/249L/251S/266L/316L/326L, 11N/12T/37L/64W/100V/101V/230T/249L/251S/266L/316L/338G/345Q, 11N/12T/37L/64W/100V/101V/230T/249L/251S/266L/316L/338L/345Q, 11N/12T/37L/197W/230T/249L/251S/266L/316L/326L, 11N/12T/37L/198M/230T/249L/251S/266L/316L/327S, 11N/12T/37L/141G/198M/230T/249L/251S/266L/316L/327S, 11N/12T/37L/100V/230T/249L/251S/266L/316L/338G, 11N/12T/37L/198M/230T/249L/251S/266L/316L/326L/327G, 11N/12T/37L/197W/230T/249L/251S/266L/316L/326L/327G, 11N/12T/37L/64W/100V/230T/249L/251S/266L/316L/338G, 11N/12T/37L/64W/100V/101V/156S/197W/230T/249L/251S/266L/316L/338L, 11N/12T/37L/64W/100V/101V/230T/249L/251S/266L/316L/338G, 11N/12T/37L/100V/197W/230T/249L/251S/266L/316L/338L, 11N/12T/37L/198M/230T/249L/251S/266L/316L/326L/327S, 11N/12T/37L/197W/230T/249L/251S/266L/316L/338L, 11N/12T/37L/101V/197W/230T/249L/251S/266L/316L/326L, 11N/12T/37L/64W/100V/197W/230T/249L/251S/266L/316L/338L, 11N/12T/37L/101V/198M/230T/249L/251S/266L/316L, 11N/12T/37L/141G/197W/230T/249L/251S/266L/316L/327S, 11N/12T/37L/198M/230T/249L/251S/266L/316L/327G, 11N/12T/37L/198M/230T/249L/251S/266L/316L/326C/327S, 11N/12T/37L/64W/100V/101V/197W/230T/249L/251S/266L/316L/338G, 11N/12T/37L/101V/230T/249L/251S/266L/316L/326L, 11N/12T/37L/101V/197W/230T/249L/251S/266L/316L/326L/327G, 11N/12T/37L/141G/197W/230T/249L/251S/266L/316L/326L/327G, 11N/12T/37L/100V/101W/230T/249L/251S/266L/316L/338G, 11N/12T/37L/197W/230T/249L/251S/266L/316L/327S, 11N/12T/37L/197W/230T/249L/251S/266L/316L, 11N/12T/37L/141G/197W/230T/249L/251S/266L/316L, 11N/12T/37L/101V/230T/249L/251S/266L/316L, 11N/12T/37L/101V/198M/230T/249L/251S/266L/316L/326C, 11N/12T/37L/64W/197W/198M/230T/249L/251S/266L/316L/338G, 11N/12T/37L/141G/230T/249L/251S/266L/316L, 11N/12T/37L/64W/100V/101W/230T/249L/251S/266L/316L/338G, 11N/12T/37L/101V/141G/230T/249L/251S/266L/316L, 11N/12T/37L/100V/101V/197W/230T/249L/251S/266L/316L/338L, 11N/12T/37L/141G/230T/249L/251S/266L/316L/326C, 11N/12T/37L/230T/249L/251S/266L/316L/338L, 11N/12T/37L/197W/230T/249L/251S/266L/316L/338G, 11N/12T/37L/230T/249L/251S/266L/316L/326L, 11N/12T/37L/197W/198M/230T/249L/251S/266L/316L/338G, 11N/12T/37L/197W/198M/230T/249L/251S/266L/316L/338L, 11N/12T/37L/64W/101W/230T/249L/251S/266L/316L/338L, 11N/12T/37L/64W/197W/198M/230T/249L/251S/266L/316L, 11N/12T/37L/101V/197W/230T/249L/251S/266L/316L, 11N/12T/37L/100V/101V/230T/249L/251S/266L/316L, 11N/12T/37L/141G/230T/249L/251S/266L/316L/326L, 11N/12T/37L/100V/109F/197W/230T/249L/251S/266L/316L/338L, 11N/12T/37L/101W/197W/230T/249L/251S/266L/316L/338L, 11N/12T/37L/64W/100V/101W/230T/249L/251S/266L/316L, 11N/12T/37L/197W/198M/230T/249L/251S/266L/316L, 11N/12T/37L/100V/101W/230T/249L/251S/266L/316L/338L, 11N/12T/37L/141G/230T/249L/251S/266L/316L/326C/327S, 11N/12T/37L/100V/197W/230T/249L/251S/266L/316L, 11N/12T/37L/197W/230T/249L/251S/266L/316L/326C/327G, 11N/12T/37L/64W/100V/230T/249L/251S/266L/316L, 11N/12T/37L/101V/230T/249L/251S/266L/316L/326C, 11N/12T/37L/100V/101W/230T/249L/251S/266L/316L, 11N/12T/37L/64W/197W/230T/249L/251S/266L/316L/338L, 11N/12T/37L/100V/197W/198M/230T/249L/251S/266L/316L, 11N/12T/37L/101W/230T/249L/251S/266L/316L, 11N/12T/37L/101W/230T/249L/251S/266L/316L/338L, 11N/12T/37L/141G/230T/249L/251S/266L/316L/327S, 11N/12T/37L/101V/230T/249L/251S/266L/306G/316L, 11N/12T/37L/64W/100V/101W/197W/230T/249L/251S/266L/316L, 11N/12T/37L/101V/230T/249L/251S/266L/316L/326L/327G, 11N/12T/37L/100V/230T/249L/251S/266L/316L, 11N/12T/37L/230T/249L/251S/266L/316L/327G, 11N/12T/37L/230T/249L/251S/266L/316L/326C/345S, 11N/12T/37L/64W/100V/101V/197W/198M/230T/249L/251S/266L/316L, 11N/12T/37L/230T/249L/251S/266L/316L/326C/327S, 11N/12T/37L/64W/101W/230T/249L/251S/266L/316L, 11N/12T/37L/100V/101V/197W/230T/249L/251S/266L/316L, 11N/12T/37L/101V/230T/249L/251S/266L/316L/327S, 11N/12T/37L/230T/249L/251S/266L/316L/338G, 11N/12T/37L/100V/101V/197W/230T/249L/251S/266L/316L/338G, 11N/12T/37L/100V/101W/197W/230T/249L/251S/266L/316L/338G, 11N/12T/37L/64W/100V/197W/198M/230T/249L/251S/266L/316L/338G, 11N/12T/37L/230T/249L/251S/266L/306G/316L, 11N/12T/37L/64W/230T/249L/251S/266L/316L/338G, 11N/12T/37L/64W/100V/101V/197W/230T/249L/251S/266L/316L, 11N/12T/37L/64W/197W/198M/230T/249L/251S/266L/316L/338L, 11N/12T/37L/101V/230T/249L/251S/266L/316L/327G, 11N/12T/37L/101V/141G/230T/249L/251S/266L/316L/326C/327S, 11N/12T/37L/64W/101W/197W/230T/249L/251S/266L/316L/338L, 11N/12T/37L/101W/230T/249L/251S/266L/316L/338G, or 11N/12T/37L/101V/198M/230T/249L/251S/266L/306G/316L/327S, wherein the amino acid positions are relative to the reference sequence corresponding to residues 12 to 346 of SEQ ID NO: 2, or relative to the reference sequence corresponding to SEQ ID NO: 2.

In some embodiments, the amino acid sequence of the engineered RNA ligase comprises at least a substitution set at amino acid positions 11/12/37/194/198/230/249/251/266/316/326/330/331, 11/12/37/198/230/249/251/266/298/316/326, 11/12/37/198/230/249/251/266/316/326/331, 11/12/37/192/193/194/198/230/249/251/266/298/316/326, 11/12/37/198/230/249/251/266/316/326/330, 11/12/37/192/198/230/249/251/266/316/326/330/331, 11/12/37/192/193/194/198/230/249/251/266/316/326/331, 11/12/37/198/230/249/251/266/298/316/326/330/331, 11/12/37/194/198/230/249/251/266/298/316/326/330/331, 11/12/37/192/193/198/230/249/251/266/316/326/330/331, 11/12/37/192/198/230/249/251/266/298/316/326/331, 11/12/37/192/193/198/230/249/251/266/316/326, 11/12/37/194/198/230/249/251/266/316/326/330, 11/12/37/60/100/156/198/230/249/251/266/316/326, 11/12/37/193/198/230/249/251/266/296/316/326/331, 11/12/37/198/230/249/251/266/298/316/326/331, 11/12/37/193/194/198/230/249/251/266/316/326/330/331, 11/12/37/194/198/230/249/251/266/316/326, 11/12/37/194/198/230/249/251/266/298/316/326/331, 11/12/37/193/198/230/249/251/266/298/316/326, 11/12/37/193/198/230/249/251/266/316/326/330, 11/12/37/192/198/230/249/251/266/316/326, 11/12/37/192/193/194/198/230/249/251/266/298/316/326/331, 11/12/37/192/193/194/198/230/249/251/266/298/316/326/330, 11/12/37/192/193/198/230/249/251/266/298/316/326/331, 11/12/37/100/156/198/230/249/251/266/316/326, 11/12/37/62/100/198/230/249/251/266/316/326, 11/12/37/198/230/249/251/266/298/316/326/330, 11/12/37/60/100/198/230/249/251/266/316/326, 11/12/37/192/193/198/230/249/251/266/298/316/326/330/331, 11/12/37/100/198/230/249/251/266/316/326, 11/12/37/193/194/198/230/249/251/266/298/316/326, 11/12/37/198/230/249/251/266/316/326/338, 11/12/37/62/198/230/249/251/266/316/326, 11/12/37/198/230/247/249/251/266/316/326, 11/12/18/37/198/230/249/251/266/316/326, 11/12/37/198/230/249/251/266/316/326, 11/12/37/198/230/249/251/254/266/316/326, 11/12/33/37/198/230/249/251/266/316/326, 11/12/37/198/230/249/251/261/266/316/326, 11/12/37/198/230/249/251/260/266/316/326, 11/12/37/117/198/230/249/251/266/316/326, 11/12/37/198/230/249/251/256/266/316/326, 11/12/37/198/230/249/251/255/266/316/326, 11/12/37/38/198/230/249/251/266/316/326, 11/37/198/230/249/251/266/316/326, 11/12/29/37/198/230/249/251/266/316/326, 11/12/25/37/198/230/249/251/266/316/326, or 11/12/37/198/230/249/251/252/266/316/326, wherein the amino acid positions are relative to the reference sequence corresponding to residues 12 to 346 of SEQ ID NO: 2, or relative to the reference sequence corresponding to SEQ ID NO: 2.

In some embodiments, the amino acid sequence of the engineered RNA ligase comprises at least a substitution set or amino acid residues 11N/12T/37L/194V/198M/230T/249L/251S/266L/316L/326L/330D/331W, 11N/12T/37L/198M/230T/249L/251S/266L/298S/316L/326L, 11N/12T/37L/198M/230T/249L/251S/266L/316L/326L/331W, 11N/12T/37L/192L/193K/194V/198M/230T/249L/251S/266L/298S/316L/326L, 11N/12T/37L/198M/230T/249L/251S/266L/316L/326L/330D, 11N/12T/37L/192L/198M/230T/249L/251S/266L/316L/326L/330D/331G, 11N/12T/37L/192L/193K/194V/198M/230T/249L/251S/266L/316L/326L/331S, 11N/12T/37L/198M/230T/249L/251S/266L/2981/316L/326L/330D/331S, 11N/12T/37L/194V/198M/230T/249L/251S/266L/298S/316L/326L/330D/331G, 11N/12T/37L/192L/193K/198M/230T/249L/251S/266L/316L/326L/330D/331S, 11N/12T/37L/192L/198M/230T/249L/251S/266L/298S/316L/326L/331S, 11N/12T/37L/192L/193K/198M/230T/249L/251S/266L/316L/326L, 11N/12T/37L/194V/198M/230T/249L/251S/266L/316L/326L/330D, 11N/12T/37L/198M/230T/249L/251S/266L/316L/326L/331G, 11N/12T/37L/192L/198M/230T/249L/251S/266L/298S/316L/326L/331W, 11N/12T/37L/60D/100R/156G/198M/230T/249L/251S/266L/316L/326L, 11N/12T/37L/193K/198M/230T/249L/251S/266L/296G/316L/326L/331W, 11N/12T/37L/198M/230T/249L/251S/266L/298S/316L/326L/331G, 11N/12T/37L/193K/194V/198M/230T/249L/251S/266L/316L/326L/330D/331W, 11N/12T/37L/194V/198M/230T/249L/251S/266L/316L/326L, 11N/12T/37L/194V/198M/230T/249L/251S/266L/298S/316L/326L/331G, 11N/12T/37L/193K/198M/230T/249L/251S/266L/298S/316L/326L, 11N/12T/37L/193K/198M/230T/249L/251S/266L/316L/326L/330D, 11N/12T/37L/192L/198M/230T/249L/251S/266L/316L/326L, 11N/12T/37L/192L/193K/194V/198M/230T/249L/251S/266L/298S/316L/326L/331S, 11N/12T/37L/192L/193K/194V/198M/230T/249L/251S/266L/298S/316L/326L/330D, 11N/12T/37L/192L/193K/198M/230T/249L/251S/266L/298S/316L/326L/331S, 11N/12T/37L/100R/156G/198M/230T/249L/251S/266L/316L/326L, 11N/12T/37L/198M/230T/249L/251S/266L/298S/316L/326L/331S, 11N/12T/37L/198M/230T/249L/251S/266L/2981/316L/326L/331G, 11N/12T/37L/62V/100V/198M/230T/249L/251S/266L/316L/326L, 11N/12T/37L/198M/230T/249L/251S/266L/298S/316L/326L/330D, 11N/12T/37L/193K/198M/230T/249L/251S/266L/2981/316L/326L, 11N/12T/37L/198M/230T/249L/251S/266L/316L/326L/331S, 11N/12T/37L/60D/100R/198M/230T/249L/251S/266L/316L/326L, 11N/12T/37L/192L/193K/198M/230T/249L/251S/266L/298S/316L/326L/330D/331S, 11N/12T/37L/192L/193K/194V/198M/230T/249L/251S/266L/298S/316L/326L/331W, 11N/12T/37L/192L/198M/230T/249L/251S/266L/316L/326L/330D/331S, 11N/12T/37L/100R/198M/230T/249L/251S/266L/316L/326L, 11N/12T/37L/193K/194V/198M/230T/249L/251S/266L/298S/316L/326L, 11N/12T/37L/194V/198M/230T/249L/251S/266L/298S/316L/326L/331S, 11N/12T/37L/198M/230T/249L/251S/266L/316L/326L/338L, 11N/12T/37L/62V/198M/230T/249L/251S/266L/316L/326L, 11N/12T/37L/198M/230T/247S/249L/251S/266L/316L/326L, 11N/12T/18H/37L/198M/230T/249L/251S/266L/316L/326L, 11N/12T/37A/198M/230T/249L/251S/266L/316L/326L, 11N/12T/37L/198M/230T/249L/251S/254S/266L/316L/326L, 11N/12T/33L/37L/198M/230T/249L/251S/266L/316L/326L, 11N/12T/37L/198M/230T/249L/251S/261T/266L/316L/326L, 11N/12T/37L/198M/230T/249L/251Y/266L/316L/326L, 11N/12T/37L/198M/230T/249L/251S/260G/266L/316L/326L, 11N/12T/37L/198M/230T/249L/251C/266L/316L/326L, 11N/12R/37L/198M/230T/249L/251S/266L/316L/326L, 11N/12T/37L/117A/198M/230T/249L/251S/266L/316L/326L, 11N/12T/37L/198M/230T/249L/251S/256S/266L/316L/326L, 11N/12T/37L/198M/230T/249L/251S/255Y/266L/316L/326L, 11N/12T/37L/198M/230T/249L/251S/255S/266L/316L/326L, 11N/12T/37L/38E/198M/230T/249L/251S/266L/316L/326L, 11N/37L/198M/230T/249L/251S/266L/316L/326L, 11N/12T/37L/198M/230T/249L/251T/266L/316L/326L, 11N/12T/37L/198M/230T/249L/251S/255V/266L/316L/326L, 11N/12T/29D/37L/198M/230T/249L/251S/266L/316L/326L, 11N/12T/37L/198M/230T/247R/249L/251S/266L/316L/326L, 11N/12T/25A/37L/198M/230T/249L/251S/266L/316L/326L, 11N/12T/37L/198M/230T/249L/251S/256E/266L/316L/326L, 11N/12T/37L/198M/230T/249L/251S/252A/266L/316L/326L, or 11N/12T/37L/198M/230T/249L/251S/261D/266L/316L/326L, wherein the amino acid positions are relative to the reference sequence corresponding to residues 12 to 346 of SEQ ID NO: 2, or relative to the reference sequence corresponding to SEQ ID NO: 2.

In some embodiments, the amino acid sequence of the engineered RNA ligase comprises at least a substitution set at amino acid positions 11/12/37/194/198/230/249/251/254/266/298/316/326/331, 11/12/18/37/194/198/230/249/251/254/266/298/316/326/331, 11/12/37/194/198/230/247/249/251/266/298/316/317/326/331, 11/12/37/194/198/230/249/251/266/298/316/317/326/331/342, 11/12/33/37/194/198/230/249/251/266/298/316/326/331, 11/12/18/37/156/194/198/230/249/251/254/266/298/316/326/331, 11/12/18/37/194/198/230/249/251/266/298/316/326/331, 11/12/37/156/194/198/230/249/251/266/298/316/326/331, 11/12/37/194/198/230/249/251/254/266/298/316/326/331/338, 11/12/37/194/198/230/249/251/254/266/298/307/316/326/331, 11/12/33/37/194/198/230/249/251/266/298/316/317/326/331, 11/12/37/194/198/230/249/251/266/298/316/326/331, 11/12/37/156/194/198/230/249/251/266/298/316/317/326/331, 11/12/37/156/194/198/230/249/251/265/266/298/316/317/326/331, 11/12/37/194/198/230/249/251/265/266/298/316/317/326/331, 11/12/37/156/194/198/230/249/251/254/266/298/316/326/331, 11/12/33/37/156/194/198/230/249/251/266/298/316/317/326/331, 11/12/37/194/198/230/249/251/266/298/316/317/326/331, 11/12/18/37/194/198/230/249/251/266/298/316/326/331/338, 11/12/37/194/198/230/249/251/266/298/316/326/331/338, 11/12/37/156/194/198/230/247/249/251/266/298/316/317/326/331, 11/12/37/194/198/230/247/249/251/265/266/298/316/317/326/331, 11/12/37/156/194/198/230/247/249/251/266/296/298/316/317/326/331, 11/12/18/37/194/198/230/249/251/266/298/307/316/326/331, 11/12/37/156/194/198/230/249/251/266/296/298/316/317/326/331, 11/12/37/156/194/198/230/247/249/251/266/298/316/326/331, 11/12/18/37/194/198/230/249/251/254/266/298/307/316/326/331, 11/12/37/194/198/230/249/251/266/298/307/316/326/331, 11/12/37/194/198/230/249/251/265/266/298/316/326/331, 11/12/18/37/156/194/198/230/249/251/266/298/316/326/331, 11/12/33/37/156/194/198/230/249/251/266/296/298/316/317/326/331, 11/12/18/37/156/194/198/230/249/251/254/266/298/316/326/331/338, 11/12/33/37/156/194/198/230/247/249/251/266/298/316/317/326/331, 11/12/37/194/198/230/249/251/266/296/298/316/326/331, 11/12/33/37/194/198/230/249/251/266/296/298/316/317/326/331, 11/12/37/156/194/198/230/249/251/266/296/298/316/326/331, 11/12/37/194/198/230/247/249/251/266/296/298/316/317/326/331, 11/12/37/194/198/230/249/251/266/296/298/316/317/326/331, 11/12/37/194/198/230/247/249/251/266/296/298/316/326/331, 11/12/33/37/156/194/198/230/249/251/266/298/316/326/331, 11/12/37/156/194/198/230/249/251/254/266/298/307/316/326/331, 11/12/18/37/156/194/198/230/249/251/266/298/307/316/326/331, or 11/12/33/37/194/198/230/249/251/265/266/298/316/326/331, wherein the amino acid positions are relative to the reference sequence corresponding to residues 12 to 346 of SEQ ID NO: 2, or relative to the reference sequence corresponding to SEQ ID NO: 2.

In some embodiments, the amino acid sequence of the engineered RNA ligase comprises at least a substitution set or amino acid residues 11N/12T/37L/194V/198M/230T/249L/251S/254S/266L/298S/316L/326L/331G, 11N/12T/18H/37L/194V/198M/230T/249L/251S/254S/266L/298S/316L/326L/331G, 11N/12T/37L/194V/198M/230T/247S/249L/251S/266L/298S/316L/317W/326L/331G, 11N/12T/37A/194V/198M/230T/249L/251S/266L/298S/316L/317W/326L/331G/342R, 11N/12T/33L/37L/194V/198M/230T/249L/251S/266L/298S/316L/326L/331G, 11N/12T/37L/194V/198M/230T/247S/249L/251S/266L/298S/316L/317F/326L/331G, 11N/12T/18H/37L/156G/194V/198M/230T/249L/251S/254S/266L/298S/316L/326L/331G, 11N/12T/18H/37L/194V/198M/230T/249L/251S/266L/298S/316L/326L/331G, 11N/12T/37L/156G/194V/198M/230T/249L/251S/266L/298S/316L/326L/331G, 11N/12T/37L/194V/198M/230T/249L/251S/254S/266L/298S/316L/326L/331G/338L, 11N/12T/37L/194V/198M/230T/249L/251S/254S/266L/298S/307R/316L/326L/331G, 11N/12T/33L/37A/194V/198M/230T/249L/251S/266L/298S/316L/317W/326L/331G, 11N/12T/37A/194V/198M/230T/249L/251S/266L/298S/316L/326L/331G, 11N/12T/37A/156G/194V/198M/230T/249L/251S/266L/298S/316L/317W/326L/331G, 11N/12T/37L/156G/194V/198M/230T/249L/251S/265F/266L/298S/316L/317W/326L/331G, 11N/12T/37L/194V/198M/230T/249L/251S/265F/266L/298S/316L/317W/326L/331G, 11N/12T/37L/156G/194V/198M/230T/249L/251S/254S/266L/298S/316L/326L/331G, 11N/12T/33L/37L/156G/194V/198M/230T/249L/251S/266L/298S/316L/317W/326L/331G, 11N/12T/37L/194V/198M/230T/249L/251S/266L/298S/316L/317F/326L/331G, 11N/12T/37L/156G/194V/198M/230T/249L/251S/266L/298S/316L/317F/326L/331G, 11N/12T/37L/194V/198M/230T/249L/251S/266L/298S/316L/317W/326L/331G, 11N/12T/37L/156G/194V/198M/230T/249L/251S/266L/298S/316L/317W/326L/331G, 11N/12T/37A/194V/198M/230T/249L/251S/266L/298S/316L/317W/326L/331G, 11N/12T/18H/37L/194V/198M/230T/249L/251S/266L/298S/316L/326L/331G/338M, 11N/12T/37L/194V/198M/230T/249L/251S/266L/298S/316L/326L/331G/338M, 11N/12T/37L/156G/194V/198M/230T/247S/249L/251S/266L/298S/316L/317W/326L/331G, 11N/12T/18H/37L/194V/198M/230T/249L/251S/266L/298S/316L/326L/331G/338L, 11N/12T/33L/37L/194V/198M/230T/249L/251S/266L/298S/316L/317W/326L/331G, 11N/12T/37A/194V/198M/230T/247S/249L/251S/265F/266L/298S/316L/317F/326L/331G, 11N/12T/37A/194V/198M/230T/249L/251S/266L/298S/316L/317F/326L/331G, 11N/12T/33L/37A/156G/194V/198M/230T/249L/251S/266L/298S/316L/317W/326L/331G, 11N/12T/37L/156G/194V/198M/230T/247S/249L/251S/266L/296W/298S/316L/317W/326L/331G, 11N/12T/18H/37L/194V/198M/230T/249L/251S/266L/298S/307R/316L/326L/331G, 11N/12T/37L/156G/194V/198M/230T/249L/251S/266L/296W/298S/316L/317W/326L/331G, 11N/12T/37L/156G/194V/198M/230T/247S/249L/251S/266L/298S/316L/326L/331G, 11N/12T/37A/194V/198M/230T/247S/249L/251S/266L/298S/316L/317F/326L/331G, 11N/12T/37L/156G/194V/198M/230T/249L/251S/265F/266L/298S/316L/317F/326L/331G, 11N/12T/18H/37L/194V/198M/230T/249L/251S/254S/266L/298S/307R/316L/326L/331G, 11N/12T/37L/194V/198M/230T/249L/251S/266L/298S/307R/316L/326L/331G, 11N/12T/37L/194V/198M/230T/249L/251S/265F/266L/298S/316L/326L/331G, 11N/12T/37L/194V/198M/230T/249L/251S/265F/266L/298S/316L/317F/326L/331G, 11N/12T/33L/37A/194V/198M/230T/249L/251S/266L/298S/316L/326L/331G, 11N/12T/18H/37L/156G/194V/198M/230T/249L/251S/266L/298S/316L/326L/331G, 11N/12T/33L/37L/156G/194V/198M/230T/249L/251S/266L/296W/298S/316L/317W/326L/331G, 11N/12T/18H/37L/156G/194V/198M/230T/249L/251S/254S/266L/298S/316L/326L/331G/338L, 11N/12T/37A/194V/198M/230T/249L/251S/265F/266L/298S/316L/317F/326L/331G, 11N/12T/33L/37A/156G/194V/198M/230T/247S/249L/251S/266L/298S/316L/317W/326L/331G, 11N/12T/37L/194V/198M/230T/249L/251S/266L/296W/298S/316L/326L/331G, 11N/12T/33L/37L/194V/198M/230T/249L/251S/266L/296W/298S/316L/317W/326L/331G, 11N/12T/37L/156G/194V/198M/230T/247S/249L/251S/266L/298S/316L/317F/326L/331G, 11N/12T/37L/156G/194V/198M/230T/249L/251S/266L/296W/298S/316L/326L/331G, 11N/12T/37L/194V/198M/230T/249L/251S/266L/298S/316L/326L/331G/338L, 11N/12T/37A/194V/198M/230T/249L/251S/265F/266L/298S/316L/317W/326L/331G, 11N/12T/37L/194V/198M/230T/247S/249L/251S/266L/296W/298S/316L/317F/326L/331G, 11N/12T/33L/37L/156G/194V/198M/230T/249L/251S/266L/298S/316L/317F/326L/331G, 11N/12T/37A/194V/198M/230T/249L/251S/266L/296W/298S/316L/317F/326L/331G, 11N/12T/37L/194V/198M/230T/247S/249L/251S/266L/296W/298S/316L/326L/331G, 11N/12T/33L/37A/156G/194V/198M/230T/249L/251S/266L/298S/316L/326L/331G, 11N/12T/37L/194V/198M/230T/249L/251S/266L/296W/298S/316L/317W/326L/331G, 11N/12T/37L/156G/194V/198M/230T/249L/251S/254S/266L/298S/307R/316L/326L/331G, 11N/12T/18H/37L/156G/194V/198M/230T/249L/251S/266L/298S/307R/316L/326L/331G, or 11N/12T/33L/37A/194V/198M/230T/249L/251S/265F/266L/298S/316L/326L/331G, wherein the amino acid positions are relative to the reference sequence corresponding to residues 12 to 346 of SEQ ID NO: 2, or relative to the reference sequence corresponding to SEQ ID NO: 2.

In some embodiments, the amino acid sequence of the engineered RNA ligase comprises at least a substitution set at amino acid positions 11/25/29/37/156/194/198/230/249/251/254/266/298/316/326/331, 11/37/194/198/230/249/251/254/255/256/257/266/298/316/326/331, 11/37/156/194/198/230/249/251/254/255/266/298/316/326/331, 11/12/37/194/198/230/247/249/251/252/254/266/298/316/326/331, 11/25/37/194/198/230/249/251/254/266/298/316/326/331, 11/37/194/198/230/249/251/254/266/298/316/326/331, 11/25/29/37/156/194/198/230/249/251/254/255/266/298/316/326/331, 11/12/37/194/198/230/249/251/254/256/266/298/316/326/331, 11/37/156/194/198/230/249/251/254/266/298/316/326/331, 11/25/37/156/194/198/230/249/251/254/255/256/257/261/266/298/316/326/331, 11/12/18/37/194/198/230/247/249/251/254/266/298/316/326/331, 11/12/25/37/194/198/230/249/251/254/255/256/257/266/298/316/326/331, 11/25/37/194/198/230/249/251/254/257/266/298/316/326/331, 11/12/37/194/198/230/249/251/254/255/257/266/298/316/326/331, 11/25/29/37/194/198/230/249/251/254/255/257/266/298/316/326/331, 11/12/37/194/198/230/249/251/254/256/257/266/298/316/326/331, 11/37/194/198/230/249/251/254/255/257/266/298/316/326/331, 11/37/156/194/198/230/249/251/254/255/256/257/266/298/316/326/331, 11/12/25/37/194/198/230/249/251/254/266/298/316/326/331, 11/12/18/37/38/194/198/230/249/251/252/254/266/298/316/326/331, 11/37/194/198/230/249/251/254/256/257/261/266/298/316/326/331, 11/29/37/194/198/230/249/251/254/266/298/316/326/331, 11/25/37/194/198/230/249/251/254/255/256/257/261/266/298/316/326/331, 11/12/37/38/194/198/230/249/251/254/266/298/316/326/331, 11/25/29/37/194/198/230/249/251/254/256/257/266/298/316/326/331, 11/12/29/37/194/198/230/249/251/254/257/266/298/316/326/331, 11/37/194/198/230/249/251/254/261/266/298/316/326/331, 11/12/37/194/198/230/249/251/254/266/298/316/326/331, 11/37/194/198/230/249/251/254/255/266/298/316/326/331, 11/29/37/156/194/198/230/249/251/254/256/257/266/298/316/326/331, 11/12/37/194/198/230/249/251/252/254/266/298/316/326/331, 11/12/37/194/198/230/249/251/254/255/257/261/266/298/316/326/331, 11/12/18/37/194/198/230/249/251/254/266/298/316/326/331, 11/12/29/37/194/198/230/249/251/254/255/257/266/298/316/326/331, 11/12/37/194/198/230/249/251/254/255/256/257/266/298/316/326/331, 11/25/29/37/156/194/198/230/249/251/254/256/257/266/298/316/326/331, 11/12/25/37/194/198/230/249/251/254/255/256/257/261/266/298/316/326/331, 11/12/29/37/194/198/230/249/251/254/255/256/257/261/266/298/316/326/331, 11/12/25/37/194/198/230/249/251/254/256/266/298/316/326/331, 11/12/18/37/38/194/198/230/247/249/251/252/254/266/298/316/326/327/331, 11/12/25/29/37/194/198/230/249/251/254/266/298/316/326/331, 11/25/37/156/194/198/230/249/251/254/255/257/266/298/316/326/331, 11/12/18/37/156/194/198/230/249/251/252/254/266/298/316/326/331, 11/37/194/198/230/249/251/254/255/261/266/298/316/326/331, 11/12/25/37/194/198/230/249/251/254/255/266/298/316/326/331, 11/37/194/198/230/249/251/254/255/256/261/266/298/316/326/331, 11/12/37/156/194/198/230/249/251/254/255/257/266/298/316/326/331, 11/12/37/156/194/198/230/249/251/254/257/266/298/316/326/331, 11/37/156/194/198/230/249/251/254/256/257/266/298/316/326/331, 11/12/25/37/156/194/198/230/249/251/254/256/257/266/298/316/326/331, 11/25/29/37/156/194/198/230/249/251/254/257/261/266/298/316/326/331, 11/12/25/37/194/198/230/249/251/254/261/266/298/316/326/331, 11/37/156/194/198/230/249/251/254/255/256/261/266/298/316/326/331, 11/25/29/37/194/198/230/249/251/254/255/256/257/266/298/316/326/331, 11/12/37/38/194/198/230/249/251/252/254/266/298/316/326/331, 11/12/37/194/198/230/249/251/254/255/266/298/316/326/331, 11/37/156/194/198/230/249/251/254/255/256/266/298/316/326/331, 11/25/29/37/194/198/230/249/251/254/261/266/298/316/326/331, 11/12/37/194/198/230/249/251/254/257/266/298/316/326/331, 11/37/194/198/230/249/251/254/255/256/266/298/316/326/331, 11/29/37/194/198/230/249/251/254/255/266/298/316/326/331, 11/37/194/198/230/249/251/254/255/257/261/266/298/316/326/331, 11/37/156/194/198/230/249/251/254/257/266/298/316/326/331, 11/12/37/192/194/198/230/249/251/252/254/266/298/316/326/331, 11/25/29/37/194/198/230/249/251/254/255/261/266/298/316/326/331, 11/37/156/194/198/230/249/251/254/255/257/266/298/316/326/331, 11/29/37/194/198/230/249/251/254/255/257/266/298/316/326/331, 11/29/37/194/198/230/249/251/254/256/257/266/298/316/326/331, 11/12/18/37/194/198/230/249/251/252/254/266/298/316/326/331, 11/12/37/156/194/198/230/249/250/251/254/266/298/316/326/331, 11/12/37/156/194/198/230/249/251/254/255/256/257/266/298/316/326/331, 11/12/25/37/194/198/230/249/251/254/257/266/298/316/326/331, 11/37/194/198/230/249/251/254/255/256/257/261/266/298/316/326/331, 11/12/25/37/156/194/198/230/249/251/254/261/266/298/316/326/331, 11/12/37/156/194/198/230/249/251/252/254/266/298/316/326/331, 11/25/37/194/198/230/249/251/254/255/256/257/266/298/316/326/331, 11/25/29/37/156/194/198/230/249/251/254/255/257/266/298/316/326/331, 11/12/29/37/156/194/198/230/249/251/254/266/298/316/326/331, 11/12/37/194/198/230/249/251/254/256/257/261/266/298/316/326/331, 11/12/25/37/156/194/198/230/249/251/254/255/256/257/266/298/316/326/331, 11/12/25/29/37/194/198/230/249/251/254/255/266/298/316/326/331, 11/29/37/156/194/198/230/249/251/254/266/298/316/326/331, 11/12/37/194/198/230/249/251/254/255/256/266/298/316/326/331, 11/12/37/38/194/198/230/249/250/251/252/254/266/298/316/326/331, 11/37/194/198/230/249/251/254/256/257/266/298/316/326/331, 11/12/37/60/194/198/230/249/251/254/266/298/316/326/331, 11/37/194/198/230/249/251/254/256/266/298/316/326/331, 11/37/156/194/198/230/249/251/254/256/266/298/316/326/331, 11/12/25/37/194/198/230/249/251/254/255/257/261/266/298/316/326/331, 11/25/29/37/194/198/230/249/251/254/255/256/261/266/298/316/326/331, 11/29/37/156/194/198/230/249/251/254/255/256/266/298/316/326/331, 11/25/37/156/194/198/230/249/251/254/255/256/266/298/316/326/331, 11/12/37/194/198/230/249/251/254/256/261/266/298/316/326/331, 11/25/29/37/156/194/198/230/249/251/254/261/266/298/316/326/331, 11/37/194/198/230/249/251/254/257/266/298/316/326/331, 11/12/37/156/194/198/230/249/251/254/255/256/261/266/298/316/326/331, 11/25/37/156/194/198/230/249/251/254/255/266/298/316/326/331, 11/25/29/37/156/194/198/230/249/251/254/255/257/261/266/298/316/326/331, 11/29/37/194/198/230/249/251/254/256/261/266/298/316/326/331, 11/12/29/37/156/194/198/230/249/251/254/261/266/298/316/326/331, 11/12/25/29/37/156/194/198/230/249/251/254/257/261/266/298/316/326/331, 11/25/37/194/198/230/249/251/254/255/256/261/266/298/316/326/331, or 11/37/156/194/198/230/249/251/254/255/261/266/298/316/326/331, wherein the amino acid positions are relative to the reference sequence corresponding to residues 12 to 346 of SEQ ID NO: 2, or relative to the reference sequence corresponding to SEQ ID NO: 2.

In some embodiments, the amino acid sequence of the engineered RNA ligase comprises at least a substitution set or amino acid residues 11N/25A/29D/37L/156G/194V/198M/230T/249L/251S/254S/266L/298S/316L/326L/331G, 11N/37L/194V/198M/230T/249L/251S/254S/255Y/256S/257D/266L/298S/316L/326L/331G, 11N/37L/156G/194V/198M/230T/249L/251S/254S/255V/266L/298S/316L/326L/331G, 11N/12T/37L/T194V/198M/230T/247R/249L/251S/252A/254S/266L/298S/316L/326L/331G, 11N/25A/37L/194V/198M/230T/249L/251S/254S/266L/298S/316L/326L/331G, 11N/37L/194V/198M/230T/249L/251S/254S/266L/298S/316L/326L/331G, 11N/25A/29D/37L/156G/194V/198M/230T/249L/251S/254S/255S/266L/298S/316L/326L/331G, 11N/12T/37L/194V/198M/230T/249L/251S/254S/256S/266L/298S/316L/326L/331G, 11N/37L/156G/194V/198M/230T/249L/251S/254S/266L/298S/316L/326L/331G, 11N/25A/37L/156G/194V/198M/230T/249L/251S/254S/255V/256S/257D/261D/266L/298S/316L/326L/331 G, 11N/12T/18H/37L/194V/198M/230T/247R/249L/251T/254S/266L/298S/316L/326L/331G, 11N/12T/25A/37L/194V/198M/230T/249L/251S/254S/255V/256S/257D/266L/298S/316L/326L/331G, 11N/25A/37L/194V/198M/230T/249L/251S/254S/257D/266L/298S/316L/326L/331G, 11N/12T/37L/194V/198M/230T/249L/251S/254S/255S/257D/266L/298S/316L/326L/331G, 11N/25A/29D/37L/194V/198M/230T/249L/251S/254S/255Y/257D/266L/298S/316L/326L/331G, 11N/12T/37L/194V/198M/230T/249L/251S/254S/256S/257D/266L/298S/316L/326L/331G, 11N/37L/194V/198M/230T/249L/251S/254S/255S/257D/266L/298S/316L/326L/331G, 11N/37L/156G/194V/198M/230T/249L/251S/254S/255N/256S/257D/266L/298S/316L/326L/331G, 11N/12T/25A/37L/194V/198M/230T/249L/251S/254S/266L/298S/316L/326L/331G, 11N/12T/18H/37L/38E/194V/198M/230T/249L/251T/252A/254S/266L/298S/316L/326L/331G, 11N/37L/194V/198M/230T/249L/251S/254S/256S/257D/261D/266L/298S/316L/326L/331G, 11N/29D/37L/194V/198M/230T/249L/251S/254S/266L/298S/316L/326L/331G, 11N/25A/37L/194V/198M/230T/249L/251S/254S/255V/256S/257D/261D/266L/298S/316L/326L/331G, 11N/12T/37L/38E/194V/198M/230T/249L/251S/254S/266L/298S/316L/326L/331G, 11N/25A/29D/37L/194V/198M/230T/249L/251S/254S/256S/257D/266L/298S/316L/326L/331G, 11N/12T/29D/37L/194V/198M/230T/249L/251S/254S/257D/266L/298S/316L/326L/331G, 11N/37L/194V/198M/230T/249L/251S/254S/261D/266L/298S/316L/326L/331G, 11N/12T/37L/194V/198M/230T/249L/251T/254S/266L/298S/316L/326L/331G, 11N/37L/156G/194V/198M/230T/249L/251S/254S/255N/266L/298S/316L/326L/331G, 11N/12T/37L/194V/198M/230T/249R/251T/254S/266L/298S/316L/326L/331G, 11N/37L/194V/198M/230T/249L/251S/254S/255V/266L/298S/316L/326L/331G, 11N/29D/37L/156G/194V/198M/230T/249L/251S/254S/256S/257D/266L/298S/316L/326L/331G, 11N/12T/37L/194V/198M/230T/249L/251S/252A/254S/266L/298S/316L/326L/331G, 11N/12T/37L/194V/198M/230T/249L/251S/254S/255V/257D/261D/266L/298S/316L/326L/331G, 11N/12T/18A/37L/194V/198M/230T/249L/251S/254S/266L/298S/316L/326L/331G, 11N/12T/29D/37L/194V/198M/230T/249L/251S/254S/255N/257D/266L/298S/316L/326L/331G, 11N/12T/37L/194V/198M/230T/249L/251S/254S/255Y/256S/257D/266L/298S/316L/326L/331G, 11N/25A/29D/37L/156G/194V/198M/230T/249L/251S/254S/256S/257D/266L/298S/316L/326L/331G, 11N/12T/25A/37L/194V/198M/230T/249L/251S/254S/255V/256S/257D/261D/266L/298S/316L/326L/331G, 11N/12T/29D/37L/194V/198M/230T/249L/251S/254S/255Y/256S/257D/261D/266L/298S/316L/326L/331G, 11N/12T/25A/37L/194V/198M/230T/249L/251S/254S/256S/266L/298S/316L/326L/331G, 11N/12T/18H/37L/38E/194V/198M/230T/247R/249L/251S/252A/254S/266L/298S/316L/326L/327M/331G, 11N/12T/18H/37L/194V/198M/230T/247R/249L/251S/254S/266L/298S/316L/326L/331G, 11N/12T/25A/29D/37L/194V/198M/230T/249L/251S/254S/266L/298S/316L/326L/331G, 11N/25A/37L/156G/194V/198M/230T/249L/251S/254S/255V/257D/266L/298S/316L/326L/331G, 11N/12T/37L/194V/198M/230T/249L/251S/254S/255V/257D/266L/298S/316L/326L/331G, 11N/37L/194V/H198M/N230T/S249L/K251S/K254S/A255N/V266L/M298S/M316L/K326L/1331G, 11N/12T/18H/37L/156G/194V/198M/230T/249L/251T/252A/254S/266L/298S/316L/326L/331G, 11N/37L/194V/198M/230T/249L/251S/254S/255V/261D/266L/298S/316L/326L/331G, 11N/12T/25A/37L/194V/198M/230T/249L/251S/254S/255V/266L/298S/316L/326L/331G, 11N/37L/194V/198M/230T/249L/251S/254S/255V/256S/261D/266L/298S/316L/326L/331G, 11N/12T/37L/156G/194V/198M/230T/249L/251S/254S/255S/257D/266L/298S/316L/326L/331G, 11N/12T/37L/156G/194V/198M/230T/249L/251S/254S/257D/266L/298S/316L/326L/331G, 11N/37L/156G/194V/198M/230T/249L/251S/254S/256S/257D/266L/298S/316L/326L/331G, 11N/37L/156G/194V/198M/230T/249L/251S/254S/255Y/266L/298S/316L/326L/331G, 11N/12T/25A/37L/156G/194V/198M/230T/249L/251S/254S/256S/257D/266L/298S/316L/326L/331G, 11N/25A/29D/37L/156G/194V/198M/230T/249L/251S/254S/257D/261D/266L/298S/316L/326L/331G, 11N/12T/25A/37L/194V/198M/230T/249L/251S/254S/261D/266L/298S/316L/326L/331G, 11N/37L/156G/194V/198M/230T/249L/251S/254S/255Y/256S/261D/266L/298S/316L/326L/331G, 11N/25A/29D/37L/194V/198M/230T/249L/251S/254S/255Y/256S/257D/266L/298S/316L/326L/331G, 11N/12T/37L/38E/194V/198M/230T/249L/251S/252A/254S/266L/298S/316L/326L/331G, 11N/37L/156G/194V/198M/230T/249L/251S/254S/255V/256S/257D/266L/298S/316L/326L/331G, 11N/12T/37L/194V/198M/230T/249L/251S/254S/255S/266L/298S/316L/326L/331G, 11N/12T/37L/194V/198M/230T/249R/251S/252A/254S/266L/298S/316L/326L/331G, 11N/37L/156G/194V/198M/230T/249L/251S/254S/255Y/256S/266L/298S/316L/326L/331G, 11N/25A/29D/37L/194V/198M/230T/249L/251S/254S/261D/266L/298S/316L/326L/331G, 11N/12T/37L/194V/198M/230T/249L/251S/254S/257D/266L/298S/316L/326L/331G, 11N/37L/194V/198M/230T/249L/251S/254S/255N/256S/266L/298S/316L/326L/331G, 11N/29D/37L/194V/198M/230T/249L/251S/254S/255N/266L/298S/316L/326L/331G, 11N/37L/194V/198M/230T/249L/251S/254S/255V/257D/261D/266L/298S/316L/326L/331G, 11N/37L/194V/198M/230T/249L/251S/254S/255Y/257D/266L/298S/316L/326L/331G, 11N/37L/156G/194V/198M/230T/249L/251S/254S/257D/266L/298S/316L/326L/331G, 11N/12T/37L/192N/194V/198M/230T/249R/251T/252A/254S/266L/298S/316L/326L/331G, 11N/25A/29D/37L/194V/198M/230T/249L/251S/254S/255N/256S/257D/266L/298S/316L/326L/331G, 11N/25A/29D/37L/194V/198M/230T/249L/251S/254S/255V/261D/266L/298S/316L/326L/331G, 11N/37L/156G/194V/198M/230T/249L/251S/254S/255Y/257D/266L/298S/316L/326L/331G, 11N/29D/37L/194V/198M/230T/249L/251S/254S/255V/257D/266L/298S/316L/326L/331G, 11N/29D/37L/194V/198M/230T/249L/251S/254S/256S/257D/266L/298S/316L/326L/331G, 11N/12T/18A/37L/194V/198M/230T/249L/251S/252A/254S/266L/298S/316L/326L/331G, 11N/12T/37L/194V/198M/230T/249R/251S/254S/266L/298S/316L/326L/331G, 11N/12T/37L/156G/194V/198M/230T/249L/250I/251S/254S/266L/298S/316L/326L/331G, 11N/12T/37L/38E/194V/198M/230T/249L/251T/252A/254S/266L/298S/316L/326L/331G, 11N/12T/37L/156G/194V/198M/230T/249L/251S/254S/255Y/256S/257D/266L/298S/316L/326L/331G, 11N/12T/25A/37L/194V/198M/230T/249L/251S/254S/257D/266L/298S/316L/326L/331G, 11N/37L/194V/198M/230T/249L/251S/254S/255V/256S/257D/261D/266L/298S/316L/326L/331G, 11N/12T/25A/37L/156G/194V/198M/230T/249L/251S/254S/261D/266L/298S/316L/326L/331G, 11N/12T/37L/156G/194V/198M/230T/249L/251S/252A/254S/266L/298S/316L/326L/331G, 11N/25A/37L/194V/198M/230T/249L/251S/254S/255V/256S/257D/266L/298S/316L/326L/331G, 11N/25A/29D/37L/156G/194V/198M/230T/249L/251S/254S/255S/257D/266L/298S/316L/326L/331G, 11N/12T/29D/37L/156G/194V/198M/230T/249L/251S/254S/266L/298S/316L/326L/331G, 11N/12T/37L/194V/198M/230T/249L/251T/252A/254S/266L/298S/316L/326L/331G, 11N/12T/37L/194V/198M/230T/249L/251S/254S/256S/257D/261D/266L/298S/316L/326L/331G, 11N/12T/25A/37L/156G/194V/198M/230T/249L/251S/254S/255Y/256S/257D/266L/298S/316L/326L/331G, 11N/37L/156G/194V/198M/230T/249L/251S/254S/255Y/256S/257D/266L/298S/316L/326L/331G, 11N/12T/37L/156G/194V/198M/230T/249R/251T/252A/254S/266L/298S/316L/326L/331G, 11N/12T/25A/29D/37L/194V/198M/230T/249L/251S/254S/255V/266L/298S/316L/326L/331G, 11N/29D/37L/156G/194V/198M/230T/249L/251S/254S/266L/298S/316L/326L/331G, 11N/12T/37L/194V/198M/230T/249L/251S/254S/255V/256S/266L/298S/316L/326L/331G, 11N/37L/194V/198M/230T/249L/251S/254S/255Y/256S/266L/298S/316L/326L/331G, 11N/12T/37L/38E/194V/198M/230T/249L/250I/251S/252A/254S/266L/298S/316L/326L/331G, 11N/12T/37L/194V/198M/230T/247R/249R/251S/252A/254S/266L/298S/316L/326L/331G, 11N/37L/194V/198M/230T/249L/251S/254S/256S/257D/266L/298S/316L/326L/331G, 11N/37L/156G/194V/198M/230T/249L/251S/254S/255S/257D/266L/298S/316L/326L/331G, 11N/37L/156G/194V/198M/230T/249L/251S/254S/255V/257D/266L/298S/316L/326L/331G, 11N/12T/37L/60S/194V/198M/230T/249L/251S/254S/266L/298S/316L/326L/331G, 11N/37L/194V/198M/230T/249L/251S/254S/255Y/256S/261D/266L/298S/316L/326L/331G, 11N/37L/194V/198M/230T/249L/251S/254S/256S/266L/298S/316L/326L/331G, 11N/37L/194V/198M/230T/249L/251S/254S/255S/266L/298S/316L/326L/331G, 11N/12T/37L/194V/198M/230T/249L/251S/254S/255S/257D/261D/266L/298S/316L/326L/331G, 11N/37L/156G/194V/198M/230T/249L/251S/254S/256S/266L/298S/316L/326L/331G, 11N/37L/194V/198M/230T/249L/251S/254S/255Y/257D/261D/266L/298S/316L/326L/331G, 11N/12T/25A/37L/194V/198M/230T/249L/251S/254S/255V/257D/261D/266L/298S/316L/326L/331G, 11N/25A/29D/37L/194V/198M/230T/249L/251S/254S/255N/256S/261D/266L/298S/316L/326L/331G, 11N/37L/194V/198M/230T/249L/251S/254S/255V/257D/266L/298S/316L/326L/331G, 11N/29D/37L/156G/194V/198M/230T/249L/251S/254S/255V/256S/266L/298S/316L/326L/331G, 11N/12T/37L/194V/198M/230T/249L/251S/254S/255S/256S/266L/298S/316L/326L/331G, 11N/25A/37L/156G/194V/198M/230T/249L/251S/254S/255V/256S/266L/298S/316L/326L/331G, 11N/12T/37L/194V/198M/230T/247R/249L/251T/252A/254S/266L/298S/316L/326L/331G, 11N/12T/37L/194V/198M/230T/249L/251S/254S/256S/261D/266L/298S/316L/326L/331G, 11N/12T/37L/38E/194V/198M/230T/249L/251T/254S/266L/298S/316L/326L/331G, 11N/25A/29D/37L/156G/194V/198M/230T/249L/251S/254S/261D/266L/298S/316L/326L/331G, 11N/37L/194V/198M/230T/249L/251S/254S/257D/266L/298S/316L/326L/331G, 11N/12T/37L/156G/194V/198M/230T/249L/251S/254S/255N/256S/261D/266L/298S/316L/326L/331G, 11N/25A/37L/156G/194V/198M/230T/249L/251S/254S/255S/266L/298S/316L/326L/331G, 11N/12T/25A/37L/194V/198M/230T/249L/251S/254S/255S/266L/298S/316L/326L/331G, 11N/25A/29D/37L/156G/194V/198M/230T/249L/251S/254S/255V/257D/261D/266L/298S/316L/326L/331G, 11N/29D/37L/194V/198M/230T/249L/251S/254S/256S/261D/266L/298S/316L/326L/331G, 11N/12T/37L/156G/194V/198M/230T/249L/251S/254S/255V/257D/266L/298S/316L/326L/331G, 11N/12T/29D/37L/156G/194V/198M/230T/249L/251S/254S/261D/266L/298S/316L/326L/331G, 11N/37L/194V/198M/230T/249L/251S/254S/255S/257D/261D/266L/298S/316L/326L/331G, 11N/12T/25A/29D/37L/156G/194V/198M/230T/249L/251S/254S/257D/261D/266L/298S/316L/326L/331G, 11N/25A/37L/194V/198M/230T/249L/251S/254S/255V/256S/261D/266L/298S/316L/326L/331G, or 11N/37L/156G/194V/198M/230T/249L/251S/254S/255V/261D/266L/298S/316L/326L/331G, wherein the amino acid positions are relative to the reference sequence corresponding to residues 12 to 346 of SEQ ID NO: 2, or relative to the reference sequence corresponding to SEQ ID NO: 2.

In some embodiments, the amino acid sequence of the engineered RNA ligase comprises at least a substitution set at amino acid positions 11/12/37/194/198/230/249/251/254/266/298/316/326/331, 11/12/28/37/194/198/230/249/251/254/266/298/316/326/331, 11/12/37/89/194/198/230/249/251/254/266/298/316/326/331, 11/12/37/95/194/198/230/249/251/254/266/298/316/326/331, 11/12/37/143/194/198/230/249/251/254/266/298/316/326/331, 11/12/37/157/194/198/230/249/251/254/266/298/316/326/331, 11/12/37/181/194/198/230/249/251/254/266/298/316/326/331, 11/12/37/183/194/198/230/249/251/254/266/298/316/326/331, 11/12/37/185/194/198/230/249/251/254/266/298/316/326/331, 11/12/37/190/194/198/230/249/251/254/266/298/316/326/331, 11/12/37/194/198/213/230/249/251/254/266/298/316/326/331, or 11/12/37/194/198/214/230/249/251/254/266/298/316/326/331, wherein the amino acid positions are relative to the reference sequence corresponding to residues 12 to 346 of SEQ ID NO: 2, or relative to the reference sequence corresponding to SEQ ID NO: 2.

In some embodiments, the amino acid sequence of the engineered RNA ligase comprises at least a substitution set or amino acid residues 11N/12T/37L/157V/194V/198M/230T/249L/251S/254S/266L/298S/316L/326L/331G, 11N/12T/37L/183L/194V/198M/230T/249L/251S/254S/266L/298S/316L/326L/331G, 11N/12T/37L/95A/194V/198M/230T/249L/251S/254S/266L/298S/316L/326L/331G, 11N/12T/37L/183V/194V/198M/230T/249L/251S/254S/266L/298S/316L/326L/331G, 11N/12T/37L/185P/194V/198M/230T/249L/251S/254S/266L/298S/316L/326L/331G, 11N/12T/37L/181E/194V/198M/230T/249L/251S/254S/266L/298S/316L/326L/331G, 11N/12T/37L/194V/198M/213D/230T/249L/251S/254S/266L/298S/316L/326L/331G, 11N/12T/37L/89M/194V/198M/230T/249L/251S/254S/266L/298S/316L/326L/331G, 11N/12T/37L/194V/198M/230T/249L/251S/254S/266L/298S/316L/326L/331G, 11N/12T/28M/37L/194V/198M/230T/249L/251S/254S/266L/298S/316L/326L/331G, 11N/12T/37L/190M/194V/198M/230T/249L/251S/254S/266L/298S/316L/326L/331G, 11N/12T/37L/194V/198M/214L/230T/249L/251S/254S/266L/298S/316L/326L/331G, 11N/12T/37L/143A/194V/198M/230T/249L/251S/254S/266L/298S/316L/326L/331G, 11N/12T/37L/89Q/194V/198M/230T/249L/251S/254S/266L/298S/316L/326L/331G, or 11N/12T/37L/95G/194V/198M/230T/249L/251S/254S/266L/298S/316L/326L/331G, wherein the amino acid positions are relative to the reference sequence corresponding to residues 12 to 346 of SEQ ID NO: 2, or relative to the reference sequence corresponding to SEQ ID NO: 2.

In some embodiments, the amino acid sequence of the engineered RNA ligase comprises at least a substitution set at amino acid positions 11/25/37/95/157/194/198/230/249/251/254/266/298/316/326/331, 11/25/37/95/183/185/194/198/214/230/249/251/254/266/298/316/326/331, 11/25/37/89/183/194/198/230/249/251/254/266/298/316/326/331, 11/25/28/37/89/143/183/194/198/230/249/251/254/266/298/316/326/331, 11/25/37/28/143/157/194/198/230/249/251/254/266/298/316/326/331, 11/25/37/181/183/194/198/230/249/251/254/266/298/316/326/331, 11/25/37/143/181/183/194/198/214/230/249/251/254/266/298/316/326/331, 11/25/37/143/181/183/194/198/230/249/251/254/266/298/316/326/331, 11/25/37/89/143/183/194/198/230/249/251/254/266/298/316/326/331, 11/25/28/37/143/181/183/194/198/230/249/251/254/266/298/316/326/331, 11/25/37/143/157/194/198/230/249/251/254/266/298/316/326/331, 11/25/37/95/181/183/194/198/230/249/251/254/266/298/316/326/331, 11/25/37/157/194/198/230/249/251/254/266/298/316/326/331, 11/25/37/143/183/194/198/230/249/251/254/266/298/316/326/331, 11/25/37/89/143/157/183/194/198/230/249/251/254/266/298/316/326/331, 11/25/37/89/95/157/194/198/230/249/251/254/266/298/316/326/331, 11/25/37/89/157/194/198/230/249/251/254/266/298/316/326/331, 11/25/37/89/95/143/181/194/198/230/249/251/254/266/298/316/326/331, 11/25/37/95/143/194/198/230/249/251/254/266/298/316/326/331, 11/25/37/89/157/183/194/198/214/230/249/251/254/266/298/316/326/331, 11/25/37/143/194/198/214/230/249/251/254/266/298/316/326/331, 11/25/37/194/198/230/249/251/254/266/298/316/326/331, 11/25/37/143/181/183/185/194/198/230/249/251/254/266/298/316/326/331, 11/25/37/143/183/194/198/214/230/249/251/254/266/298/316/326/331, 11/25/28/37/95/183/194/198/214/230/249/251/254/266/298/316/326/331, 11/25/37/89/143/157/194/198/230/249/251/254/266/298/316/326/331, 11/25/28/37/95/194/198/230/249/251/254/266/298/316/326/331, 11/25/37/95/143/183/185/194/198/230/249/251/254/266/298/316/326/331, or 11/25/37/95/143/157/183/194/198/230/249/251/254/266/298/316/326/331, wherein the amino acid positions are relative to the reference sequence corresponding to residues 12 to 346 of SEQ ID NO: 2, or relative to the reference sequence corresponding to SEQ ID NO: 2.

In some embodiments, the amino acid sequence of the engineered RNA ligase comprises at least a substitution set or amino acid residues 11N/25A/37L/95A/157V/194V/198M/230T/249L/251S/254S/266L/298S/316L/326L/331G, 11N/25A/37L/95G/183V/185P/194V/198M/214L/230T/249L/251S/254S/266L/298S/316L/326L/331G, 11N/25A/37L/89M/183V/194V/198M/230T/249L/251S/254S/266L/298S/316L/326L/331G, 11N/25A/28M/37L/89G/143M/183L/194V/198M/230T/249L/251S/254S/266L/298S/316L/326L/331G, 11N/25A/37L/28M/143M/157V/194V/198M/230T/249L/251S/254S/266L/298S/316L/326L/331G, 11N/25A/37L/181E/183V/194V/198M/230T/249L/251S/254S/266L/298S/316L/326L/331G, 11N/25A/37L/143A/181E/183V/194V/198M/214L/230T/249L/251S/254S/266L/298S/316L/326L/331G, 11N/25A/37L/143A/181E/183V/194V/198M/230T/249L/251S/254S/266L/298S/316L/326L/331G, 11N/25A/37L/89G/143M/183L/194V/198M/230T/249L/251S/254S/266L/298S/316L/326L/331G, 11N/25A/28M/37L/143M/181E/183L/194V/198M/230T/249L/251S/254S/266L/298S/316L/326L/331G, 11N/25A/37L/143M/157V/194V/198M/230T/249L/251S/254S/266L/298S/316L/326L/331G, 11N/25A/37L/95A/181E/183L/194V/198M/230T/249L/251S/254S/266L/298S/316L/326L/331G, 11N/25A/37L/157V/194V/198M/230T/249L/251S/254S/266L/298S/316L/326L/331G, 11N/25A/37L/143A/183L/194V/198M/230T/249L/251S/254S/266L/298S/316L/326L/331G, 11N/25A/37L/143A/157V/194V/198M/230T/249L/251S/254S/266L/298S/316L/326L/331G, 11N/25A/37L/89G/143A/157V/183L/194V/198M/230T/249L/251S/254S/266L/298S/316L/326L/331G, 11N/25A/37L/89M/95A/157V/194V/198M/230T/249L/251S/254S/266L/298S/316L/326L/331G, 11N/25A/37L/89M/157V/194V/198M/230T/249L/251S/254S/266L/298S/316L/326L/331G, 11N/25A/37L/89G/95A/143A/181E/194V/198M/230T/249L/251S/254S/266L/298S/316L/326L/331G, 11N/25A/37L/95A/143A/194V/198M/230T/249L/251S/254S/266L/298S/316L/326L/331G, 11N/25A/37L/89M/157V/183L/194V/198M/214L/230T/249L/251S/254S/266L/298S/316L/326L/331G, 11N/25A/37L/143M/194V/198M/214L/230T/249L/251S/254S/266L/298S/316L/326L/331G, 11N/25A/37L/194V/198M/230T/249L/251S/254S/266L/298S/316L/326L/331G, 11N/25A/37L/143M/181E/183V/185P/194V/198M/230T/249L/251S/254S/266L/298S/316L/326L/331G, 11N/25A/37L/143M/183L/194V/198M/214L/230T/249L/251S/254S/266L/298S/316L/326L/331G, 11N/25A/28M/37L/95A/183V/194V/198M/214L/230T/249L/251S/254S/266L/298S/316L/326L/331G, 11N/25A/37L/89M/143A/157V/194V/198M/230T/249L/251S/254S/266L/298S/316L/326L/331G, 11N/25A/28M/37L/95A/194V/198M/230T/249L/251S/254S/266L/298S/316L/326L/331G, 11N/25A/37L/95A/143M/183V/185P/194V/198M/230T/249L/251S/254S/266L/298S/316L/326L/331G, or 11N/25A/37L/95A/143A/157V/183L/194V/198M/230T/249L/251S/254S/266L/298S/316L/326L/331G, wherein the amino acid positions are relative to the reference sequence corresponding to residues 12 to 346 of SEQ ID NO: 2, or relative to the reference sequence corresponding to SEQ ID NO: 2.

In some embodiments, the amino acid sequence of the engineered RNA ligase comprises at least a substitution set at amino acid positions 11/25/37/83/89/137/150/157/183/194/198/214/230/249/251/254/266/298/316/326/331, 11/25/37/89/157/183/194/198/213/214/230/249/251/254/266/298/316/326/331, 11/25/37/89/137/157/183/194/198/214/230/249/251/254/266/298/316/326/331, 11/25/37/89/150/157/183/190/194/198/214/230/249/251/254/266/298/316/326/331, 11/25/37/89/143/150/157/183/194/198/214/230/249/251/254/266/298/316/326/331, 11/25/37/60/89/143/150/157/183/190/194/198/214/230/249/251/254/266/298/316/326/331, 11/25/37/60/89/150/157/183/190/194/198/213/214/230/249/251/254/266/298/316/326/331, 11/25/37/89/104/137/154/157/183/194/198/214/230/249/251/254/266/298/316/326/331, 11/25/37/89/123/137/157/183/194/198/214/230/249/251/254/266/298/316/326/331, 11/25/37/89/137/150/154/157/183/190/194/198/214/230/249/251/254/266/298/316/326/331, 11/25/37/89/123/143/150/157/183/194/198/214/230/249/251/254/266/298/316/326/331, 11/25/37/60/89/104/143/150/157/183/194/198/213/214/230/249/251/254/266/298/316/326/331, 11/25/37/60/89/95/143/157/183/190/194/198/214/230/249/251/254/266/298/316/326/331, 11/25/37/89/123/137/157/183/190/194/198/214/230/249/251/254/266/298/316/326/331, 11/25/37/60/89/123/157/183/194/198/214/230/249/251/254/266/298/316/326/331, 11/25/37/89/104/123/157/183/194/198/214/230/249/251/254/266/298/316/326/331, or 11/25/37/60/89/123/143/157/183/194/198/214/230/249/251/254/266/298/316/326/331, wherein the amino acid positions are relative to the reference sequence corresponding to residues 12 to 346 of SEQ ID NO: 2, or relative to the reference sequence corresponding to SEQ ID NO: 2.

In some embodiments, the amino acid sequence of the engineered RNA ligase comprises at least a substitution set or amino acid residue 11N/25A/37L/83L/89M/137R/150D/157V/183L/194V/198M/214L/230T/249L/251S/254S/266L/298S/316L/326L/331G, 11N/25A/37R/89M/157V/183L/194V/198M/213R/214L/230T/249L/251S/254S/266L/298S/316L/326L/331G, 11N/25A/37L/89M/137R/157V/183L/194V/198M/214L/230T/249L/251S/254S/266L/298S/316L/326L/331G, 11N/25A/37L/89M/150D/157V/183L/190M/194V/198M/214L/230T/249L/251S/254S/266L/298S/316L/326L/331G, 11N/25A/37L/89M/143A/150D/157V/183L/194V/198M/214L/230T/249L/251S/254S/266L/298S/316L/326L/331G, 11N/25A/37L/89M/143T/150D/157V/183L/194V/198M/214L/230T/249L/251S/254S/266L/298S/316L/326L/331G, 11N/25A/37L/60A/89M/143A/150D/157V/183L/190M/194V/198M/214L/230T/249L/251S/254S/266L/298S/316L/326L/331G, 11N/25A/37L/60A/89M/150D/157V/183L/190M/194V/198M/213R/214L/230T/249L/251S/254S/266L/298S/316L/326L/331G, 11N/25A/37L/89M/104T/137R/154V/157V/183L/194V/198M/214L/230T/249L/251S/254S/266L/298S/316L/326L/331G, 11N/25A/37L/89M/123K/137R/157V/183L/194V/198M/214L/230T/249L/251S/254S/266L/298S/316L/326L/331G, 11N/25A/37L/89M/137R/150D/154V/157V/183L/190M/194V/198M/214L/230T/249L/251S/254S/266L/298 S/316L/326L/331G, 11N/25A/37L/89M/123K/143T/150D/157V/183L/194V/198M/214L/230T/249L/251S/254S/266L/298S/316L/326L/331G, 11N/25A/37L/60A/89M/104T/143A/150D/157V/I183L/194V/198M/213R/214L/230T/249L/251S/254S/266L/298S/316L/326L/331G, 11N/25A/37L/60A/89M/95G/143T/157V/183L/190M/194V/198M/214L/230T/249L/251S/254S/266L/298S/316L/326L/331G, 11N/25A/37L/89M/123R/137R/157V/183L/190M/194V/198M/214L/230T/249L/251S/254S/266L/298S/316L/326L/331G, 11N/25A/37L/60A/89M/123R/157V/183L/194V/198M/214L/230T/249L/251S/254S/266L/298S/316L/326L/331G, 11N/25A/37L/89M/104T/123R/157V/183L/194V/198M/214L/230T/249L/251S/254S/266L/298S/316L/326L/331G, or 11N/25A/37L/60A/89M/123R/143A/157V/183L/194V/198M/214L/230T/249L/251S/254S/266L/298S/316L/326L/331G, wherein the amino acid positions are relative to the reference sequence corresponding to residues 12 to 346 of SEQ ID NO: 2, or relative to the reference sequence corresponding to SEQ ID NO: 2.

In some embodiments, the amino acid sequence of the engineered RNA ligase comprises at least a substitution set at amino acid positions 11/25/37/38/89/157/183/194/198/214/230/249/251/254/266/298/316/326/331, 11/25/37/89/157/183/194/198/214/230/249/251/254/266/298/316/326/331, 11/25/37/89/157/183/194/198/214/230/249/251/254/255/266/298/316/326/331, 11/23/25/37/89/157/183/194/198/214/230/249/251/254/266/298/316/326/331, 11/25/33/37/89/157/183/194/198/214/230/249/251/254/266/298/316/326/331, 11/25/37/89/157/183/194/198/214/226/230/249/251/254/266/298/316/326/331, 11/25/37/89/157/183/194/198/214/230/251/254/266/298/316/326/331, 11/25/29/37/89/157/183/194/198/214/230/249/251/254/266/298/316/326/331, 10/11/25/37/89/157/183/194/198/214/230/249/251/254/266/298/316/326/331, 11/25/37/89/157/183/194/198/214/230/249/250/251/254/266/298/316/326/331, 11/25/37/75/89/157/183/194/198/214/230/249/251/254/266/298/316/326/331, 11/25/37/77/89/157/183/194/198/214/230/249/251/254/266/298/316/326/331, 11/25/37/89/157/183/194/198/214/228/230/249/251/254/266/298/316/326/331, 11/25/37/81/89/157/183/194/198/214/230/249/251/254/266/298/316/326/331, 11/25/37/89/157/183/194/198/214/224/230/249/251/254/266/298/316/326/331, 11/25/37/64/89/157/183/194/198/214/230/249/251/254/266/298/316/326/331, 11/25/37/89/157/183/194/198/214/230/249/251/266/298/316/326/331, 11/25/37/89/157/183/194/198/214/230/249/251/254/266/268/298/316/326/331, 11/25/37/65/89/157/183/194/198/214/230/249/251/254/266/298/316/326/331, 11/25/37/89/157/183/194/198/214/230/232/249/251/254/266/298/316/326/331, 11/25/37/89/157/183/194/198/214/230/249/251/254/266/298/316/326/331/332, 11/25/37/89/157/183/194/198/214/230/249/251/254/266/298/316/326/330/331, 11/25/37/89/101/157/183/194/198/214/230/249/251/254/266/298/316/326/331, 11/25/37/89/157/183/194/198/214/230/249/251/254/266/298/307/316/326/331, 11/25/37/89/137/157/183/194/198/214/230/249/251/254/266/298/316/326/331, 11/25/37/89/157/183/194/198/214/223/230/249/251/254/266/298/316/326/331, 11/25/37/89/157/183/194/198/214/230/249/251/254/266/280/298/316/326/331, 11/25/37/89/157/183/194/198/214/230/249/251/254/266/289/298/316/326/331, 11/25/37/89/157/183/194/198/214/230/249/251/254/266/288/298/316/326/331, 11/25/37/89/157/183/194/198/214/230/249/251/254/266/298/316/326/331/342, 11/25/37/89/93/157/183/194/198/214/230/249/251/254/266/298/316/326/331, 11/25/37/89/157/183/194/198/214/230/241/249/251/254/266/298/316/326/331, 11/25/37/89/126/157/183/194/198/214/230/249/251/254/266/298/316/326/331, 11/25/37/89/157/183/194/198/214/230/249/251/254/266/277/298/316/326/331, 11/25/37/87/89/157/183/194/198/214/230/249/251/254/266/298/316/326/331, 11/25/37/89/157/183/194/198/214/230/247/249/251/254/266/298/316/326/331, 11/25/37/89/157/183/194/198/214/230/243/249/251/254/266/298/316/326/331, 11/25/37/84/89/157/183/194/198/214/230/249/251/254/266/298/316/326/331, 11/25/37/83/89/157/183/194/198/214/230/249/251/254/266/298/316/326/331, 11/25/37/89/157/183/192/194/198/214/230/249/251/254/266/298/316/326/331, 11/25/37/89/105/157/183/194/198/214/230/249/251/254/266/298/316/326/331, 11/25/29/37/89/157/183/194/198/214/250/230/249/251/254/266/298/316/326/331, 11/25/29/37/75/77/89/157/183/194/198/214/230/249/250/251/254/255/266/298/316/326/331, 11/25/37/77/89/157/183/194/198/214/226/230/249/251/254/255/266/298/316/326/331, 11/25/37/89/157/183/194/198/214/230/249/251/254/250/255/266/298/316/326/331, 11/25/29/37/75/89/157/183/194/198/214/226/230/249/251/254/255/266/298/316/326/331, 11/25/29/37/75/77/89/157/183/194/198/214/230/249/251/254/266/298/316/326/331, 11/25/29/37/75/77/89/157/183/194/198/214/226/230/249/251/254/266/298/316/326/331, 11/25/29/37/75/77/89/157/183/194/198/214/230/249/251/254/255/266/298/316/326/331, 11/25/29/37/89/157/183/194/198/214/226/230/249/251/254/266/298/316/326/331, 11/25/29/37/89/157/183/194/198/214/230/249/251/254/255/266/298/316/326/331, 11/25/33/37/89/157/183/194/198/214/228/230/249/251/254/266/298/316/326/331, 11/25/33/37/89/157/183/194/198/214/230/251/254/266/298/316/326/331, 11/25/33/37/38/89/157/183/194/198/214/228/230/249/251/254/266/298/316/326/331, 11/25/33/37/38/89/157/183/194/198/214/230/249/251/254/266/298/316/326/331, 11/25/37/75/89/157/183/194/198/214/230/249/250/251/254/255/266/298/316/326/331, 11/25/37/75/89/157/183/194/198/214/226/230/249/251/254/266/298/316/326/331, 11/25/37/75/89/157/183/194/198/214/226/230/249/250/251/254/255/266/298/316/326/331, 11/25/37/75/77/89/157/183/194/198/214/230/249/251/254/266/298/316/326/331, 11/25/37/75/77/89/157/183/194/198/214/230/249/251/254/255/266/298/316/326/331, 11/25/37/75/77/89/157/183/194/198/214/230/249/250/251/254/255/266/298/316/326/331, 11/25/37/75/77/89/157/183/194/198/214/226/230/249/250/251/254/255/266/298/316/326/331, 11/25/37/75/77/89/157/183/194/198/214/230/249/250/251/254/266/298/316/326/331, 11/25/37/75/89/157/183/194/198/214/230/249/251/254/255/266/298/316/326/331, 11/25/37/75/89/157/183/194/198/214/230/249/250/251/254/266/298/316/326/331, 11/25/37/89/157/183/194/198/214/226/230/249/251/254/255/266/298/316/326/331, 11/25/37/89/157/183/194/198/214/228/230/251/254/266/298/316/326/331, 11/25/37/38/89/157/183/194/198/214/228/230/249/251/254/266/298/316/326/331, 11/25/37/38/89/157/183/194/198/214/230/251/254/266/291/298/316/326/331, 10/11/25/37/89/157/183/194/198/214/228/230/249/251/254/266/298/316/326/331, 10/11/25/33/37/89/157/183/194/198/214/230/249/251/254/266/298/316/326/331, 10/11/25/33/37/38/89/157/183/194/198/214/230/249/251/254/266/298/316/326/331, 10/11/25/33/37/38/89/157/183/194/198/214/230/251/254/266/298/316/326/331, 10/11/25/33/37/89/157/183/194/198/214/228/230/249/251/254/266/298/316/326/331, 10/11/25/33/37/89/157/183/194/198/214/230/251/254/266/298/316/326/331, 10/11/25/33/37/38/89/157/183/194/198/214/228/230/249/251/254/266/298/316/326/331, 10/11/25/37/89/157/183/194/198/214/230/251/254/266/298/316/326/331, 10/11/25/37/38/89/157/183/194/198/214/228/230/249/251/254/266/298/316/326/331, 10/11/25/37/38/89/157/183/194/198/214/230/249/251/254/266/298/316/326/331, 10/11/25/37/38/89/157/183/194/198/214/228/230/251/254/266/298/316/326/331, 11/25/37/77/89/157/183/194/198/214/230/249/250/251/254/266/298/316/326/331, 11/25/37/77/89/157/183/194/198/214/230/249/250/251/254/255/266/298/316/326/331, 11/25/37/77/89/157/183/194/198/214/226/230/249/251/254/266/298/316/326/331, 77/11/25/37/89/157/183/194/198/214/226/230/249/251/254/255/266/298/316/326/331, 11/25/37/77/89/157/183/194/198/214/230/249/251/254/255/266/298/316/326/331, 11/25/37/89/157/183/194/198/214/230/249/250/251/254/255/266/298/316/326/331, 11/23/25/37/89/157/183/194/198/214/226/230/249/251/254/255/266/298/316/326/331, or 11/23/25/37/89/157/183/194/198/214/226/230/249/251/254/266/298/316/326/331, wherein the amino acid positions are relative to the reference sequence corresponding to residues 12 to 346 of SEQ ID NO: 2, or relative to the reference sequence corresponding to SEQ ID NO: 2.

In some embodiments, the amino acid sequence of the engineered RNA ligase comprises at least a substitution set or amino acid residue 11N/25A/37L/38V/89M/157V/183L/194V/198M/214L/230T/249L/251S/254S/266L/298S/316L/326L/331G, 11N/25A/37L/89M/157V/183L/194V/198M/214L/230T/249K/251S/254S/266L/298S/316L/326L/331G, 11N/25A/37L/89M/157V/183L/194V/198M/214L/230T/249L/251S/254S/255S/266L/298S/316L/326L/331G, 11N/23K/25A/37L/89M/157V/183L/194V/198M/214L/230T/249L/251S/254S/266L/298S/316L/326L/331G, 11N/25A/33L/37L/89M/157V/183L/194V/198M/214L/230T/249L/251S/254S/266L/298S/316L/326L/331G, 11N/25G/37L/89M/157V/183L/194V/198M/214L/230T/249L/251S/254S/266L/298S/316L/326L/331G, 11N/25A/37L/89M/157V/183L/194V/198M/214L/226R/230T/249L/251S/254S/266L/298S/316L/326L/331G, 11N/25A/37L/89M/157V/183L/194V/198M/214L/230T/251S/254S/266L/298S/316L/326L/331G, 11N/25A/29S/37L/89M/157V/183L/194V/198M/214L/230T/249L/251S/254S/266L/298S/316L/326L/331G, 11N/25A/37M/89M/157V/183L/194V/198M/214L/230T/249L/251S/254S/266L/298S/316L/326L/331G, 11N/25A/37L/38A/89M/157V/183L/194V/198M/214L/230T/249L/251S/254S/266L/298S/316L/326L/331G, 10F/11N/25A/37L/89M/157V/183L/194V/198M/214L/230T/249L/251S/254S/266L/298S/316L/326L/331G, 11N/25A/37L/89M/157V/183L/194V/198M/214L/230T/249L/250S/251S/254S/266L/298S/316L/326L/331G, 11N/25A/37L/75D/89M/157V/183L/194V/198M/214L/230T/249L/251S/254S/266L/298S/316L/326L/331G, 11N/25A/37L/89M/157V/183L/194V/198M/214L/230V/249L/251S/254S/266L/298S/316L/326L/331G, 11N/25A/37L/77M/89M/157V/183L/194V/198M/214L/230T/249L/251S/254S/266L/298S/316L/326L/331G, 11N/25A/37L/89M/157V/183L/194V/198M/214L/230T/249L/251E/254S/266L/298S/316L/326L/331G, 11N/23T/25A/37L/89M/157V/183L/194V/198M/214L/230T/249L/251S/254S/266L/298S/316L/326L/331G, 11N/25A/33V/37L/89M/157V/183L/194V/198M/214L/230T/249L/251S/254S/266L/298S/316L/326L/331G, 11N/25A/37L/89M/157V/183L/194V/198M/214L/228L/230T/249L/251S/254S/266L/298S/316L/326L/331G, 11N/25A/37L/81Y/89M/157V/183L/194V/198M/214L/230T/249L/251S/254S/266L/298S/316L/326L/331G, 11N/25A/37L/89M/157V/183L/194V/198M/214L/224A/230T/249L/251S/254S/266L/298S/316L/326L/331G, 11N/25A/37L/64Q/89M/157V/183L/194V/198M/214L/230T/249L/251S/254S/266L/298S/316L/326L/331G, 11N/25A/37L/89M/157V/183L/194V/198M/214L/230T/249L/251S/266L/298S/316L/326L/331G, 11N/25A/37L/89M/157V/183L/194V/198M/214L/230T/249L/251S/254S/266L/268F/298S/316L/326L/331G, 11N/25A/37L/65C/89M/157V/183L/194V/198M/214L/230T/249L/251S/254S/266L/298S/316L/326L/331G, 11N/25A/37L/89M/157V/183L/194V/198M/214L/230T/232N/249L/251S/254S/266L/298S/316L/326L/331G, 11N/25A/37L/89M/157V/183L/194V/198M/214L/230T/249L/251S/254S/266L/298S/316L/326L/331G/332E, 11N/25A/37K/89M/157V/183L/194V/198M/214L/230T/249L/251S/254S/266L/298S/316L/326L/331G, 11N/25A/37L/89M/157V/183L/194V/198M/214L/230T/249L/251S/254S/266L/298S/316L/326L/331R, 11N/25A/37L/89M/157V/183L/194V/198M/214L/230T/249L/251S/254S/266L/298S/316L/326L/330K/331G, 11N/25A/37L/89M/101P/157V/183L/194V/198M/214L/230T/249L/251S/254S/266L/298S/316L/326L/331G, 11N/25A/37L/89M/157V/183L/194V/198M/214L/230T/249L/251S/254S/266L/268G/298S/316L/326L/331G, 11N/25A/37L/89M/157V/183L/194V/198M/214L/230T/249L/251S/254S/266L/298S/307R/316L/326L/331G, 11N/25A/37L/89M/157V/183L/194V/198M/214L/224Q/230T/249L/251S/254S/266L/298S/316L/326L/331G, 11N/25A/37L/89M/137N/157V/183L/194V/198M/214L/230T/249L/251S/254S/266L/298S/316L/326L/331G, 11N/25A/37L/89M/157V/183L/194V/198M/214L/223C/230T/249L/251S/254S/266L/298S/316L/326L/331G, 11N/25A/37L/89M/157V/183L/194V/198M/214L/230T/249L/251S/254S/266L/280N/298S/316L/326L/331G, 11N/25A/37L/89M/157V/183L/194V/198M/214L/230T/249L/251S/254S/266L/2891/298S/316L/326L/331G, 11N/25A/37L/89M/157V/183L/194V/198M/214L/230T/249L/251S/254S/266L/288E/298S/316L/326L/331G, 11N/25A/37L/89M/157V/183L/194V/198M/214L/230T/249L/251S/254S/266L/298S/316L/326L/331G/342T, 11N/25A/37L/89M/93K/157V/183L/194V/198M/214L/230T/249L/251S/254S/266L/298S/316L/326L/331G, 11N/25A/37L/89M/157V/183L/194V/198M/214L/230T/249L/251S/254S/266L/298S/316L/326L/331G, 11N/25A/37L/89M/157V/183L/194V/198M/214L/230T/249L/251S/254S/266L/268M/298S/316L/326L/331G, 11N/25A/37L/89M/157V/183L/194V/198M/214L/230T/249L/251S/254S/266L/298S/316L/326L/331G, 11N/25A/37L/89M/157V/183L/194V/198M/214L/230T/241Q/249L/251S/254S/266L/298S/316L/326L/331G, 11N/25A/37L/89M/126M/157V/183L/194V/198M/214L/230T/249L/251S/254S/266L/298S/316L/326L/331G 11N/25A/37L/89M/157V/183L/194V/198M/214L/230T/249L/251S/254S/266L/277A/298S/316L/326L/331G, 11N/25A/37L/87E/89M/157V/183L/194V/198M/214L/230T/249L/251S/254S/266L/298S/316L/326L/331G, 11N/25A/37L/89M/157V/183L/194V/198M/214L/230T/247I/249L/251S/254S/266L/298S/316L/326L/331G, 11N/25A/37L/89M/157V/183L/194V/198M/214L/230T/243H/249L/251S/254S/266L/298S/316L/326L/331G, 11N/25A/37L/84V/89M/157V/183L/194V/198M/214L/230T/249L/251S/254S/266L/298S/316L/326L/331G, 11N/25A/37L/83A/89M/157V/183L/194V/198M/214L/230T/249L/251S/254S/266L/298S/316L/326L/331G, 11N/25A/37L/89M/157V/183L/192E/194V/198M/214L/230T/249L/251S/254S/266L/298S/316L/326L/331G, 11N/25A/37L/89M/105K/157V/183L/194V/198M/214L/230T/249L/251S/254S/266L/298S/316L/326L/331G, 11N/25G/29S/37L/89M/157V/183L/194V/198M/214L/230T/249L/251S/254S/266L/298S/316L/326L/331G, 11N/25G//29S/37L/89M/157V/183L/194V/198M/214L/250S/230T/249L/251S/254S/266L/298S/316L/326L/331G, 11N/25G/29S/37L/75D/77M/89M/157V/183L/194V/198M/214L/230T/249L/250S/251S/254S/255S/266L/298 S/316L/326L/331G, 11N/25G/37L/75D/89M/157V/183L/194V/198M/214L/230V/249L/251S/254S/266L/298S/316L/326L/331G, 11N/25G/37L/77M/89M/157V/183L/194V/198M/214L/226R/230T/249L/251S/254S/255S/266L/298S/316L/326L/331G, 11N/25G/37L/89M/157V/183L/194V/198M/214L/230V/249L/251S/254S/266L/298S/316L/326L/331G, 11N/25A/37L/89M/157V/183L/194V/198M/214L/230T/249L/251S/254S/250S/255S/266L/298S/316L/326L/331G, 11N/25A/29S/37L/75D/89M/157V/183L/194V/198M/214L/226R/230V/249L/251S/254S/255S/266L/298S/316L/326L/331G, 11N/25A/29S/37L/75D/77M/89M/157V/183L/194V/198M/214L/230T/249L/251S/254S/266L/298S/316L/326 L/331G, 11N/25A/29S/37L/75D/77M/89M/157V/183L/194V/198M/214L/226R/230T/249L/251S/254S/266L/298S/316L/326L/331G, 11N/25A/29S/37L/75D/77M/89M/157V/183L/194V/198M/214L/230V/249L/251S/254S/255S/266L/298S/316L/326L/331G, 11N/25A/29S/37L/89M/157V/183L/194V/198M/214L/226R/230T/249L/251S/254S/266L/298S/316L/326L/331G, 11N/25A/29S/37L/89M/157V/183L/194V/198M/214L/226R/230V/249L/251S/254S/266L/298S/316L/326L/I 331G, 11N/25A/29S/37L/89M/157V/183L/194V/198M/214L/230V/249L/251S/254S/255S/266L/298S/316L/326L/13 31G, 11N/25A/33V/37L/89M/157V/183L/194V/198M/214L/228L/230T/249L/251S/254S/266L/298S/316L/326L/331G, 11N/25A/33V/37L/89M/157V/183L/194V/198M/214L/230T/249K/251S/254S/266L/298S/316L/326L/331G, 11N/25A/33V/37L/89M/157V/183L/194V/198M/214L/230T/251S/254S/266L/298S/316L/326L/331G, 11N/25A/33V/37M/89M/157V/183L/194V/198M/214L/228L/230T/249L/251S/254S/266L/298S/316L/326L/331G, 11N/25A/33V/37M/38A/89M/157V/183L/194V/198M/214L/228L/230T/249L/251S/254S/266L/298S/316L/326L/331G, 11N/25A/33V/37M/38A/89M/157V/183L/194V/198M/214L/228L/230T/249K/251S/254S/266L/298S/316L/326L/331G, 11N/25A/33V/37L/38V/89M/157V/183L/194V/198M/214L/230T/249L/251S/254S/266L/298S/316L/326L/331G, 11N/25A/37L/75D/89M/157V/183L/194V/198M/214L/230T/249L/250S/251S/254S/255S/266L/298S/316L/326L/331G, 11N/25A/37L/75D/89M/157V/183L/194V/198M/214L/226R/230T/249L/251S/254S/266L/298S/316L/326L/331G, 11N/25A/37L/75D/89M/157V/183L/194V/198M/214L/226R/230T/249L/250S/251S/254S/255S/266L/298S/316L/326L/331G, 11N/25A/37L/75D/89M/157V/183L/194V/198M/214L/226R/230V/249L/250S/251S/254S/255S/266L/298S/316L/326L/331G, 11N/25A/37L/75D/77M/89M/157V/183L/194V/198M/214L/230T/249L/251S/254S/266L/298S/316L/326L/331G, 11N/25A/37L/75D/77M/89M/157V/183L/194V/198M/214L/230T/249L/251S/254S/255S/266L/298S/316L/326L/331G, 11N/25A/37L/75D/77M/89M/157V/183L/194V/198M/214L/230T/249L/250S/251S/254S/255S/266L/298S/316L/326L/331G, 11N/25A/37L/89M/157V/183L/194V/198M/214L/230T/249L/251S/254S/266L/298S/316L/326L/331G, 11N/25A/37L/75D/77M/89M/157V/183L/194V/198M/214L/226R/230T/249L/250S/251S/254S/255S/266L/298S/316L/326L/331G, 11N/25A/37L/75D/77M/89M/157V/183L/194V/198M/214L/230V/249L/251S/254S/266L/298S/316L/326L/331G, 11N/25A/37L/75D/77M/89M/157V/183L/194V/198M/214L/230V/249L/250S/251S/254S/266L/298S/316L/326L/331G, 11N/25A/37L/75D/89M/157V/183L/194V/198M/214L/230V/249L/251S/254S/266L/298S/316L/326L/331G, 11N/25A/37L/75D/89M/157V/183L/194V/198M/214L/230V/249L/251S/254S/255S/266L/298S/316L/326L/331G, 11N/25A/37L/75D/89M/157V/183L/194V/198M/214L/230V/249L/250S/251S/254S/266L/298S/316L/326L/331G, 11N/25A/37L/89M/157V/183L/194V/198M/214L/226R/230T/249L/251S/254S/266L/298S/316L/326L/331G, 11N/25A/37L/89M/157V/183L/194V/198M/214L/230T/249L/251S/254S/255S/266L/298S/316L/326L/331G, 11N/25A/37L/89M/157V/183L/194V/198M/214L/226R/230V/249L/251S/254S/266L/298S/316L/326L/331G, 11N/25A/37L/89M/157V/183L/194V/198M/214L/226R/230V/249L/251S/254S/255S/266L/298S/316L/326L/331G, 11N/25A/37M/89M/157V/183L/194V/198M/214L/228L/230T/249L/251S/254S/266L/298S/316L/326L/331G, 11N/25A/37M/89M/157V/183L/194V/198M/214L/228L/230T/251S/254S/266L/298S/316L/326L/331G, 11N/25A/37M/38A/89M/157V/183L/194V/198M/214L/230T/249L/251S/254S/266L/298S/316L/326L/331G, 11N/25A/37M/38A/89M/157V/183L/194V/198M/214L/228L/230T/249L/251S/254S/266L/298S/316L/326L/331G, 11N/25A/37M/38V/89M/157V/183L/194V/198M/214L/228L/230T/249K/251S/254S/266L/298S/316L/326L/331G, 11N/25A/37M/38V/89M/157V/183L/194V/198M/214L/230T/249K/251S/254S/266L/298S/316L/326L/331G, 11N/25A/37L/38A/89M/157V/183L/194V/198M/214L/230T/251S/254S/266L/291T/298S/316L/326L/331G, 10F/11N/25A/37L/89M/157V/183L/194V/198M/214L/228L/230T/249L/251S/254S/266L/298S/316L/326L/331G, 10F/11N/25A/37L/89M/157V/183L/194V/198M/214L/228L/230T/249K/251S/254S/266L/298S/316L/326L/331G, 10F/11N/25A/33L/37L/89M/157V/183L/194V/198M/214L/230T/249K/251S/254S/266L/298S/316L/326L/331G, 10F/11N/25A/33L/37M/89M/157V/183L/194V/198M/214L/230T/249L/251S/254S/266L/298S/316L/326L/331G, 10F/11N/25A/33L/37M/38A/89M/157V/183L/194V/198M/214L/230T/249K/251S/254S/266L/298S/316L/326L/331G, 10F/11N/25A/33L/37L/38A/89M/157V/183L/194V/198M/214L/230T/251S/254S/266L/298S/316L/326L/331 G, 10F/11N/25A/33L/37L/38V/89M/157V/183L/194V/198M/214L/230T/249L/251S/254S/266L/298S/316L/326 L/331G, 10F/11N/25A/33V/37L/89M/157V/183L/194V/198M/214L/230T/249K/251E/254S/266L/298S/316L/326L/331G, 10F/11N/25A/33V/37M/89M/157V/183L/194V/198M/214L/230T/249L/251S/254S/266L/298S/316L/326L/331G, 10F/11N/25A/33V/37M/89M/157V/183L/194V/198M/214L/228L/230T/249L/251S/254S/266L/298S/316L/326L/331G, 10F/11N/25A/33V/37M/89M/157V/183L/194V/198M/214L/230T/249K/251S/254S/266L/298S/316L/326L/331G, 10F/11N/25A/33V/37M/89M/157V/183L/194V/198M/214L/230T/251S/254S/266L/298S/316L/326L/331G, 10F/11N/25A/33V/37M/38A/89M/157V/183L/194V/198M/214L/228L/230T/249K/251S/254S/266L/298S/316L/326L/331G, 10F/11N/25A/33V/37M/38V/89M/157V/183L/194V/198M/214L/230T/249L/251S/254S/266L/298S/316L/326L/331G, 10F/11N/25A/33V/37M/38V/89M/157V/183L/194V/198M/214L/228L/230T/249K/251S/254S/266L/298S/316L/326L/331G, 10F/11N/25A/33V/37M/38V/89M/157V/183L/194V/198M/214L/230T/249K/251S/254S/266L/298S/316L/326L/331G, 10F/11N/25A/37L/89M/157V/183L/194V/198M/214L/230T/249K/251S/254S/266L/298S/316L/326L/331G, 10F/11N/25A/37L/89M/157V/183L/194V/198M/214L/230T/251S/254S/266L/298S/316L/326L/331G, 10F/11N/25A/37M/89M/157V/183L/194V/198M/214L/230T/249L/251S/254S/266L/298S/316L/326L/331G, 10F/11N/25A/37M/89M/157V/183L/194V/198M/214L/228L/230T/249L/251S/254S/266L/298S/316L/326L/331G, 10F/11N/25A/37M/89M/157V/183L/194V/198M/214L/228L/230T/249K/251S/254S/266L/298S/316L/326L/331G, 10F/11N/25A/37M/89M/157V/183L/194V/198M/214L/230T/249K/251S/254S/266L/298S/316L/326L/331G, 10F/11N/25A/37M/38A/89M/157V/183L/194V/198M/214L/228L/230T/249L/251S/254S/266L/298S/316L/326L/331G, 10F/11N/25A/37M/38A/89M/157V/183L/194V/198M/214L/228L/230T/249K/251S/254S/266L/298S/316L/326L/331G, 10F/11N/25A/37M/38V/89M/157V/183L/194V/198M/214L/230T/249L/251S/254S/266L/298S/316L/326L/331G, 10F/11N/25A/37M/38V/89M/157V/183L/194V/198M/214L/228L/230T/249L/251S/254S/266L/298S/316L/326L/331G, 10F/11N/25A/37M/38V/89M/157V/183L/194V/198M/214L/228L/230T/251S/254S/266L/298S/316L/326L/331G, 10F/11N/25A/37L/38A/89M/157V/183L/194V/198M/214L/230T/249K/251S/254S/266L/298S/316L/326L/331G, 10F/11N/25A/37L/38V/89M/157V/183L/194V/198M/214L/230T/249L/251S/254S/266L/298S/316L/326L/331G, 10F/11N/25A/37L/38V/89M/157V/183L/194V/198M/214L/230T/249L/251S/254S/266L/298S/316L/326L/331G, 11N/25A/37L/89M/157V/183L/194V/198M/214L/228L/230T/249L/251S/254S/266L/298S/316L/326L/331G, 11N/25A/37L/89M/157V/183L/194V/198M/214L/230T/249L/251S/254S/266L/298S/316L/326L/331G, 11N/25A/37L/77M/89M/157V/183L/194V/198M/214L/230T/249L/250S/251S/254S/266L/298S/316L/326L/331G, 11N/25A/37L/77M/89M/157V/183L/194V/198M/214L/230T/249L/250S/251S/254S/255S/266L/298S/316L/326L/331G, 11N/25A/37L/77M/89M/157V/183L/194V/198M/214L/226R/230T/249L/251S/254S/266L/298S/316L/326L/331G, 77M/11N/25A/37L/89M/157V/183L/194V/198M/214L/226R/230T/249L/251S/254S/255S/266L/298S/316L/3 26L/331G, 11N/25A/37L/77M/89M/157V/183L/194V/198M/214L/230V/249L/251S/254S/266L/298S/316L/326L/331G, 11N/25A/37L/77M/89M/157V/183L/194V/198M/214L/230V/249L/251S/254S/255S/266L/298S/316L/326L/331G, 11N/25A/37L/89M/157V/183L/194V/198M/214L/230V/249L/251S/254S/255S/266L/298S/316L/326L/331G, 11N/25A/37L/89M/157V/183L/194V/198M/214L/230V/249L/250S/251S/254S/266L/298S/316L/326L/331G, 11N/25A/37L/89M/157V/183L/194V/198M/214L/230V/249L/250S/251S/254S/255S/266L/298S/316L/326L/331G, 11N/23T/25G/37L/89M/157V/183L/194V/198M/214L/226R/230T/249L/251S/254S/255S/266L/298S/316L/326L/331G, 11N/23T/25G/37L/89M/157V/183L/194V/198M/214L/226R/230V/249L/251S/254S/266L/298S/316L/326L/331G, or 11N/23T/25G/37L/89M/157V/183L/194V/198M/214L/230V/249L/251S/254S/266L/298S/316L/326L/331G, wherein the amino acid positions are relative to the reference sequence corresponding to residues 12 to 346 of SEQ ID NO: 2, or relative to the reference sequence corresponding to SEQ ID NO: 2.

In some embodiments, the amino acid sequence of the engineered RNA ligase comprises at least a substitution set at amino acid positions 11/25/37/86/89/157/183/194/198/214/230/249/251/254/266/298/316/326/331, 11/25/37/89/157/183/194/198/214/230/249/251/254/255/266/298/316/326/331, 11/25/37/89/157/183/194/198/214/230/249/251/254/266/298/306/316/326/331, 11/25/37/89/157/183/194/198/214/230/249/250/251/254/266/298/316/326/331, 11/25/37/75/89/157/183/194/198/214/230/249/251/254/266/298/316/326/331, 11/25/37/89/157/183/194/198/214/230/249/251/254/266/298/316/318/326/331, 11/25/37/89/157/183/194/198/214/230/249/251/254/266/298/316/326/331/335, 11/25/37/89/157/183/194/198/214/241/230/249/251/254/266/298/316/326/331, 11/25/37/89/157/183/194/198/214/230/248/249/251/254/266/298/316/326/331, 11/25/37/89/157/183/194/198/214/230/249/251/254/266/298/316/326/331/344, 11/25/37/38/89/157/183/194/198/214/230/249/251/254/266/298/316/326/331, 11/25/37/89/157/183/194/198/214/230/249/251/254/266/272/298/316/326/331, 11/25/37/83/89/157/183/194/198/214/230/249/251/254/266/298/316/326/331, 11/25/37/89/157/183/194/198/214/230/249/251/254/257/266/298/316/326/331, 11/25/37/89/157/183/194/198/214/230/249/251/254/258/266/298/316/326/331, 11/25/37/89/157/183/194/198/214/230/249/251/254/266/298/316/323/326/331, wherein the amino acid positions are relative to the reference sequence corresponding to residues 12 to 346 of SEQ ID NO: 2, or relative to the reference sequence corresponding to SEQ ID NO: 2.

In some embodiments, the amino acid sequence of the engineered RNA ligase comprises at least a substitution set or amino acid residue 11N/25A/37L/86D/89M/157V/183L/194V/198M/214L/230T/249L/251S/254S/266L/298S/316L/326L/331G, 11N/25A/37L/89M/157V/183L/194V/198M/214L/230T/249L/251S/254S/255V/266L/298S/316L/326L/331G, 11N/25A/37L/89M/157V/183L/194V/198M/214L/230T/249L/251S/254S/266L/298S/306T/316L/326L/331G, 11N/25A/37L/89M/157V/183L/194V/198M/214L/230T/249L/250I/251S/254S/266L/298S/316L/326L/331G, 11N/25A/37L/75A/89M/157V/183L/194V/198M/214L/230T/249L/251S/254S/266L/298S/316L/326L/331G, 11N/25A/37L/89M/157V/183L/194V/198M/214L/230T/249L/251S/254S/266L/298S/316L/318S/326L/331G, 11N/25A/37L/89M/157V/183L/194V/198M/214L/230T/249L/251S/254S/266L/298S/316L/326L/331G/335Q, 11N/25A/37L/89M/157V/183L/194V/198M/214L/241G/230T/249L/251S/254S/266L/298S/316L/326L/331G, 11N/25A/37L/89M/157V/183L/194V/198M/214L/230T/249L/250E/251S/254S/266L/298S/316L/326L/331G, 11N/25A/37L/89M/157V/183L/194V/198M/214L/230T/248F/249L/251S/254S/266L/298S/316L/326L/331G, 11N/25A/37L/89M/157V/183L/194V/198M/214L/230T/249L/251S/254S/266L/298S/316L/326L/331G/344K, 11N/25A/37L/38S/89M/157V/183L/194V/198M/214L/230T/249L/251S/254S/266L/298S/316L/326L/331G, 11N/25A/37L/89M/157V/183L/194V/198M/214L/230T/249L/251S/254S/266L/272S/298S/316L/326L/331G, 11N/25A/37L/83G/89M/157V/183L/194V/198M/214L/230T/249L/251S/254S/266L/298S/316L/326L/331G, 11N/25A/37L/89M/157V/183L/194V/198M/214L/230T/249L/251S/254S/266L/298S/306L/316L/326L/331G, 11N/25A/37L/89M/157V/183L/194V/198M/214L/230T/249L/251S/254S/266L/272M/298S/316L/326L/331G, 11N/25A/37L/89M/157V/183L/194V/198M/214L/230T/248G/249L/251S/254S/266L/298S/316L/326L/331G, 11N/25A/37L/89M/157V/183L/194V/198M/214L/230T/249L/251S/254S/257E/266L/298S/316L/326L/331G, 11N/25A/37L/89M/157V/183L/194V/198M/214L/230T/249L/251S/254S/258M/266L/298S/316L/326L/331G, 11N/25A/37L/89M/157V/183L/194V/198M/214L/230T/249L/251S/254S/266L/298S/316L/323G/326L/331G, wherein the amino acid positions are relative to the reference sequence corresponding to residues 12 to 346 of SEQ ID NO: 2, or relative to the reference sequence corresponding to SEQ ID NO: 2.

In some embodiments, the amino acid sequence of the engineered RNA ligase comprises at least a substitution set at amino acid positions 11/25/29/37/89/101/157/183/194/198/214/226/230/249/251/254/266/298/316/326/331, 11/25/29/37/89/157/183/194/198/214/226/230/249/251/254/266/291/298/316/326/331, 11/25/29/37/89/157/183/194/198/214/226/230/249/251/254/266/291/298/307/316/326/331, 11/25/29/37/89/157/183/194/198/214/226/230/249/251/254/266/298/316/326/331, 11/25/29/33/37/75/89/157/183/194/198/214/226/230/249/251/254/266/298/316/326/331, 11/25/29/33/37/75/89/157/183/194/198/214/226/230/249/251/254/266/291/298/316/326/331, 11/25/29/33/37/38/89/157/183/194/198/214/226/230/249/251/254/266/298/316/326/331, 11/25/29/33/37/89/93/157/183/194/198/214/226/230/249/251/254/266/298/316/326/331, 11/25/29/37/38/75/89/157/183/194/198/214/226/230/249/251/254/266/291/298/307/316/326/331, 11/25/29/37/38/75/89/157/183/194/198/214/226/230/249/250/251/254/266/298/316/326/331, 11/25/29/37/38/75/89/93/157/183/194/198/214/226/230/249/251/254/266/271/298/316/326/331, 11/25/29/37/38/89/93/157/183/194/198/214/226/230/249/251/254/266/298/316/326/331, 10/11/25/29/37/75/89/157/183/194/198/214/226/230/249/251/254/266/298/316/326/331, 10/11/25/29/37/38/75/89/157/183/194/198/214/226/230/249/251/254/266/298/316/326/331, 10/11/25/29/37/38/75/89/157/183/194/198/214/226/230/249/251/254/266/291/298/316/326/331, 10/11/25/29/37/38/75/89/93/157/183/194/198/214/226/230/249/251/254/266/298/316/326/331, 11/25/29/37/64/89/157/183/194/198/214/224/226/230/249/251/254/266/298/316/326/331, 11/25/29/37/89/157/183/194/198/214/224/226/230/249/251/254/266/298/316/326/331, 11/23/25/29/37/89/101/157/183/194/198/214/226/230/249/251/254/266/298/307/316/326/331, 11/23/25/29/37/89/157/183/194/198/214/226/230/249/251/254/266/268/298/316/326/331, 11/23/25/29/37/64/89/157/183/194/198/214/224/226/230/249/251/266/298/316/326/331, 11/23/25/29/37/64/81/89/157/183/194/198/214/226/230/249/251/254/266/298/316/326/331, 11/23/25/29/37/89/157/183/194/198/214/224/226/230/249/251/254/266/268/298/316/326/331, 11/23/25/29/37/89/157/183/194/198/214/224/226/230/249/251/266/298/316/326/331, 11/23/25/29/37/89/157/183/194/198/214/226/230/249/251/254/266/298/316/326/331, 11/23/25/29/37/64/89/157/183/194/198/214/224/226/230/249/251/254/266/298/316/326/331, 11/25/29/37/64/89/157/183/194/198/214/226/230/249/251/254/266/298/316/326/331, 11/25/29/37/75/89/157/183/194/198/214/226/230/249/251/254/266/298/307/316/326/331, 11/25/29/37/89/93/157/183/194/198/214/226/230/249/251/254/266/298/316/326/331, 11/25/29/33/37/38/75/89/157/183/194/198/214/226/230/249/251/254/266/298/316/326/331, 11/25/29/37/75/89/157/183/194/198/214/226/230/249/251/254/266/298/316/326/331, 11/25/29/37/38/75/89/93/157/183/194/198/214/226/230/249/251/254/266/291/298/316/326/331, 11/23/25/29/37/89/157/183/194/198/214/226/230/249/251/266/298/316/326/331, wherein the amino acid positions are relative to the reference sequence corresponding to residues 12 to 346 of SEQ ID NO: 2, or relative to the reference sequence corresponding to SEQ ID NO: 2.

In some embodiments, the amino acid sequence of the engineered RNA ligase comprises at least a substitution set or amino acid residues 11N/25A/29S/37L/89M/101P/157V/183L/194V/198M/214L/226R/230V/249L/251S/254S/266L/298S/316L/326L/331G, 11N/25A/29S/37L/89M/157V/183L/194V/198M/214L/226R/230V/249L/251S/254S/266L/291T/298S/316L/326L/331G, 11N/25A/29S/37L/89M/157V/183L/194V/198M/214L/226R/230V/249L/251S/254S/266L/291T/298S/307R/316L/326L/331G, 11N/25A/29S/37L/89M/157V/183L/194V/198M/214L/226R/230V/249L/251S/254S/266L/298S/316L/326L/331R, 11N/25A/29S/33V/37L/75D/89M/157V/183L/194V/198M/214L/226R/230V/249L/251S/254S/266L/298S/316L/326L/331G, 11N/25A/29S/33V/37L/75D/89M/157V/183L/194V/198M/214L/226R/230V/249K/251S/254S/266L/291T/298S/316L/326L/331R, 11N/25A/29S/33V/37L/38A/89M/157V/183L/194V/198M/214L/226R/230V/249L/251S/254S/266L/298S/316L/326L/331G, 11N/25A/29S/33V/37L/89M/93K/157V/183L/194V/198M/214L/226R/230V/249K/251S/254S/266L/298S/316L/326L/331G, 11N/25A/29S/37L/38A/75D/89M/157V/183L/194V/198M/214L/226R/230V/249L/251S/254S/266L/291T/29 8S/307R/316L/326L/331G, 11N/25A/29S/37L/38V/75D/89M/157V/183L/194V/198M/214L/226R/230V/249K/250S/251S/254S/266L/298S/316L/326L/331G, 11N/25A/29S/37L/38V/75D/89M/93K/157V/183L/194V/198M/214L/226R/230V/249L/251S/254S/266L/271 G/298S/316L/326L/331G, 11N/25A/29S/37L/38V/89M/93K/157V/183L/194V/198M/214L/226R/230V/249L/251S/254S/266L/298S/316L/326L/331G, 10F/11N/25A/29S/37L/75D/89M/157V/183L/194V/198M/214L/226R/230V/249L/251S/254S/266L/298S/316L/326L/331G, 10F/11N/25A/29S/37L/38A/75D/89M/157V/183L/194V/198M/214L/226R/230V/249L/251S/254S/266L/298 S/316L/326L/331G, 10F/11N/25A/29S/37L/38V/75D/89M/157V/183L/194V/198M/214L/226R/230V/249L/251S/254S/266L/291 T/298S/316L/326L/331G, 10F/11N/25A/29S/37L/38V/75D/89M/157V/183L/194V/198M/214L/226R/230V/249K/251S/254S/266L/298 S/316L/326L/331G, 10F/11N/25A/29S/37L/38V/75D/89M/93K/157V/183L/194V/198M/214L/226R/230V/249L/251S/254S/266L/298S/316L/326L/331G, 11N/25A/29S/37L/64Q/89M/157V/183L/194V/198M/214L/224A/226R/230V/249L/251S/254S/266L/298S/316L/326L/331G, 11N/25A/29S/37L/89M/157V/183L/194V/198M/214L/224A/226R/230V/249L/251S/254S/266L/298S/316L/326L/331G, 11N/25A/29S/37L/89M/157V/183L/194V/198M/214L/224Q/226R/230V/249L/251S/254S/266L/298S/316L/326L/331G, 11N/23T/25A/29S/37L/89M/101P/157V/183L/194V/198M/214L/226R/230V/249L/251S/254S/266L/298S/307K/316L/326L/331G, 11N/23T/25A/29S/37L/89M/157V/183L/194V/198M/214L/226R/230V/249L/251S/254S/266L/268F/298S/316L/326L/331G, 11N/23T/25A/29S/37K/64Q/89M/157V/183L/194V/198M/214L/224A/226R/230V/249L/251S/266L/298S/316L/326L/331G, 11N/23T/25A/29S/37L/64Q/81Y/89M/157V/183L/194V/198M/214L/226R/230V/249L/251S/254S/266L/298 S/316L/326L/331G, 11N/23T/25A/29S/37L/89M/157V/183L/194V/198M/214L/224A/226R/230V/249L/251S/254S/266L/268F/298S/316L/326L/331G, 11N/23T/25A/29S/37L/89M/157V/183L/194V/198M/214L/224A/226R/230V/249L/251S/266L/298S/316L/326L/331G, 11N/23T/25A/29S/37L/89M/157V/183L/194V/198M/214L/226R/230V/249L/251S/254S/266L/298S/316L/326L/331G, 11N/23T/25A/29S/37L/64Q/89M/157V/183L/194V/198M/214L/224A/226R/230V/249L/251S/254S/266L/298S/316L/326L/331G, 11N/25A/29S/37L/64Q/89M/157V/183L/194V/198M/214L/226R/230V/249L/251S/254S/266L/298S/316L/326L/331G, 11N/25A/29S/37L/75D/89M/157V/183L/194V/198M/214L/226R/230V/249K/251S/254S/266L/298S/307R/316L/326L/331G, 11N/25A/29S/37L/89M/93K/157V/183L/194V/198M/214L/226R/230V/249L/251S/254S/266L/298S/316L/326L/331G, 11N/25A/29S/33V/37L/38A/75D/89M/157V/183L/194V/198M/214L/226R/230V/249K/251S/254S/266L/298 S/316L/326L/331G, 11N/25A/29S/37L/75D/89M/157V/183L/194V/198M/214L/226R/230V/249K/251S/254S/266L/298S/316L/326L/331G, 11N/25S/29S/37L/38V/75D/89M/93K/157V/183L/194V/198M/214L/226R/230V/249L/251S/254S/266L/291 T/298S/316L/326L/331G, 11N/25A/29S/33V/37L/38A/75D/89M/157V/183L/194V/198M/214L/226R/230V/249L/251S/254S/266L/298 S/316L/326L/331G, 11N/25A/29S/37L/75D/89M/157V/183L/194V/198M/214L/226R/230V/249L/251S/254S/266L/298S/316L/326L/331G, 11N/23T/25A/29S/37L/89M/157V/183L/194V/198M/214L/226R/230V/249L/251S/266L/298S/316L/326L/331G, wherein the amino acid positions are relative to the reference sequence corresponding to residues 12 to 346 of SEQ ID NO: 2, or relative to the reference sequence corresponding to SEQ ID NO: 2.

In some embodiments, the amino acid sequence of the engineered RNA ligase comprises at least a substitution set at amino acid positions 11/23/25/29/37/38/65/89/157/183/192/194/198/214/226/230/249/251/266/298/316/326/331, 11/23/25/29/37/38/65/89/157/183/194/198/214/226/230/249/251/266/298/316/326/331, 11/23/25/29/37/38/65/89/157/183/194/198/214/224/226/230/249/251/266/298/316/326/331, 11/23/25/29/37/38/64/89/157/183/194/198/214/226/230/249/251/266/298/316/326/331, 11/23/25/29/37/89/93/157/183/194/198/214/226/230/249/251/266/298/307/316/326/331, 11/23/25/29/37/38/89/157/183/194/198/214/226/230/249/251/266/298/316/326/331, 11/23/25/29/37/38/89/157/183/194/198/214/224/226/230/249/251/266/298/316/326/331, 11/23/25/29/37/38/89/157/183/192/194/198/214/224/226/230/249/251/266/298/316/326/331, 11/23/25/29/37/65/89/157/183/194/198/214/226/230/249/251/266/298/316/326/331, 11/23/25/29/37/89/157/183/192/194/198/214/224/226/230/249/251/266/298/316/326/331, 11/23/25/29/37/75/89/157/183/194/198/214/226/230/249/251/266/298/307/316/326/331, 11/23/25/29/37/75/89/157/183/194/198/214/226/230/249/250/251/266/268/298/307/316/326/331, 11/23/25/29/37/89/157/183/194/198/214/226/230/249/250/251/266/268/298/307/316/326/331/342, 11/23/25/29/37/89/157/183/194/198/214/224/226/230/249/251/266/298/316/326/331, 11/23/25/29/37/65/89/157/183/192/194/198/214/226/230/249/251/266/298/316/326/331, 11/23/25/29/37/89/157/183/194/198/214/226/230/249/251/266/268/298/316/326/331, 11/23/25/29/37/64/89/157/183/192/194/198/214/226/230/249/251/266/298/316/326/331, 11/23/25/29/37/89/157/183/194/198/214/226/230/249/250/251/266/268/291/298/316/326/331/342, 11/23/25/29/37/75/89/93/157/183/194/198/214/226/230/249/251/266/298/307/316/326/331/342, 11/23/25/29/37/89/157/183/194/198/214/226/230/249/250/251/266/291/298/316/326/331, 11/23/25/29/37/38/64/89/157/183/192/194/198/214/226/230/249/251/266/298/316/326/331, 11/23/25/29/37/38/64/89/157/183/194/198/214/224/226/230/249/251/266/298/316/326/331, 11/23/25/29/37/38/64/86/89/157/183/192/194/198/214/226/230/241/249/251/266/298/316/326/331, 11/23/25/29/37/38/89/157/183/194/198/214/226/230/249/251/266/298/316/326/331//342, 11/23/25/29/37/89/157/183/194/198/214/226/230/248/249/250/251/266/298/307/316/326/331, 11/23/25/29/37/75/77/89/157/183/194/198/214/226/230/249/250/251/266/268/298/307/316/326/331/342, 11/23/25/29/37/89/93/157/183/194/198/214/226/230/249/251/266/268/298/316/326/331, 11/23/25/29/37/89/157/183/192/194/198/214/226/230/249/251/266/298/316/326/331, 11/23/25/29/37/38/89/157/183/192/194/198/214/226/230/249/251/266/298/316/326/331, 11/23/25/29/37/64/89/157/183/194/198/214/226/230/249/251/266/298/316/326/331, wherein the amino acid positions are relative to the reference sequence corresponding to residues 12 to 346 of SEQ ID NO: 2, or relative to the reference sequence corresponding to SEQ ID NO: 2.

In some embodiments, the amino acid sequence of the engineered RNA ligase comprises at least a substitution set or amino acid residue 11N/23T/25A/29S/37L/38A/65C/89M/157V/183L/192E/194V/198M/214L/226R/230V/249L/251S/266L/298 S/316L/326L/331G, 11N/23T/25A/29S/37L/38T/65C/89M/157V/183L/194V/198M/214L/226R/230V/249L/251S/266L/298S/316 L/326L/331G, 11N/23T/25A/29S/37L/38A/65C/89M/157V/183L/194V/198M/214L/224R/226R/230V/249L/251S/266L/298 S/316L/326L/331G, 11N/23T/25A/29S/37L/38A/64Q/89M/157V/183L/194V/198M/214L/226R/230V/249L/251S/266L/298S/316 L/326L/331G, 11N/23T/25A/29S/37L/89M/93K/157V/183L/194V/198M/214L/226R/230V/249L/251S/266L/298S/307R/316L/326L/331G, 11N/23T/25A/29S/37L/38A/89M/157V/183L/194V/198M/214L/226R/230V/249L/251S/266L/298S/316L/326L/331G, 11N/23T/25A/29S/37L/38A/89M/157V/183L/194V/198M/214L/224A/226R/230V/249L/251S/266L/298S/316L/326L/331G, 11N/23T/25A/29S/37L/38A/89M/157V/183L/192E/194V/198M/214L/224R/226R/230V/249L/251S/266L/298S/316L/326L/331G, 11N/23T/25A/29S/37L/65C/89M/157V/183L/194V/198M/214L/226R/230V/249L/251S/266L/298S/316L/326L/331G, 11N/23T/25A/29S/37L/38A/89M/157V/183L/192E/194V/198M/214L/224A/226R/230V/249L/251S/266L/298S/316L/326L/331G, 11N/23T/25A/29S/37L/89M/157V/183L/192E/194V/198M/214L/224A/226R/230V/249L/251S/266L/298S/316L/326L/331G, 11N/23T/25A/29S/37L/75D/89M/157V/183L/194V/198M/214L/226R/230V/249L/251S/266L/298S/307R/31 6L/326L/331G, 11N/23T/25A/29S/37L/75D/89M/157V/183L/194V/198M/214L/226R/230V/249L/250I/251S/266L/268G/298S/307R/316L/326L/331G, 11N/23T/25A/29S/37L/89M/157V/183L/194V/198M/214L/226R/230V/249L/250I/251S/266L/268G/298S/307R/316L/326L/331G/342T, 11N/23T/25A/29S/37L/89M/157V/183L/194V/198M/214L/224R/226R/230V/249L/251S/266L/298S/316L/326L/331G, 11N/23T/25A/29S/37L/65C/89M/157V/183L/192E/194V/198M/214L/226R/230V/249L/251S/266L/298S/316L/326L/331G, 11N/23T/25A/29S/37L/89M/157V/183L/194V/198M/214L/226R/230V/249L/251S/266L/268G/298S/316L/326L/331G, 11N/23T/25A/29S/37L/64Q/89M/157V/183L/192E/194V/198M/214L/226R/230V/249L/251S/266L/298S/316L/326L/331G, 11N/23T/25A/29S/37L/89M/157V/183L/194V/198M/214L/226R/230V/249L/2501/251S/266L/268G/291T/298S/316L/326L/331G/342T, 11N/23T/25A/29S/37L/75D/89M/93K/157V/183L/194V/198M/214L/226R/230V/249K/251S/266L/298S/307R/316L/326L/331G/342T, 11N/23T/25A/29S/37L/89M/157V/183L/194V/198M/214L/226R/230V/249L/250I/251S/266L/291T/298S/316L/326L/331G, 11N/23T/25A/29S/37L/38A/64Q/89M/157V/183L/192E/194V/198M/214L/226R/230V/249L/251S/266L/298 S/316L/326L/331G, 11N/23T/25A/29S/37L/38A/64Q/89M/157V/183L/194V/198M/214L/224R/226R/230V/249L/251S/266L/298 S/316L/326L/331G, 11N/23T/25A/29S/37L/89M/93K/157V/183L/194V/198M/214L/226R/230V/249K/251S/266L/298S/307R/316L/326L/331G, 11N/23T/25A/29S/37L/38A/64Q/86G/89M/157V/183L/192E/194V/198M/214L/226R/230V/241R/249L/251 S/266L/298S/316L/326L/331G, 11N/23T/25A/29S/37L/38A/89M/157V/183L/194V/198M/214L/226R/230V/249L/251S/266L/298S/316L/326L/331G/342T, 11N/23T/25A/29S/37L/89M/157V/183L/192E/194V/198M/214L/224R/226R/230V/249L/251S/266L/298S/316L/326L/331G, 11N/23T/25A/29S/37L/89M/157V/183L/194V/198M/214L/226R/230V/248G/249L/250I/251S/266L/298S/307R/316L/326L/331G, 11N/23T/25A/29S/37L/75D/77M/89M/157V/183L/194V/198M/214L/226R/230V/249L/250I/251S/266L/268 G/298S/307R/316L/326L/331G/342T, 11N/23T/25A/29S/37L/89M/93K/157V/183L/194V/198M/214L/226R/230V/249L/251S/266L/268G/298S/316L/326L/331G, 11N/23T/25A/29S/37L/89M/157V/183L/192E/194V/198M/214L/226R/230V/249L/251S/266L/298S/316L/326L/331G, 11N/23T/25A/29S/37L/38A/89M/157V/183L/192E/194V/198M/214L/226R/230V/249L/251S/266L/298S/316L/326L/331G, 11N/23T/25A/29S/37L/64Q/89M/157V/183L/194V/198M/214L/226R/230V/249L/251S/266L/298S/316L/326L/331G, wherein the amino acid positions are relative to the reference sequence corresponding to residues 12 to 346 of SEQ ID NO: 2, or relative to the reference sequence corresponding to SEQ ID NO: 2.

In some embodiments, the amino acid sequence of the engineered RNA ligase comprises at least a substitution set at amino acid positions 11/23/25/29/37/38/75/89/157/183/194/198/214/226/230/248/249/250/251/266/268/298/307/316/326/331, 11/23/25/29/37/38/75/89/157/183/194/198/214/226/230/248/249//250/250/251/266/268/277/298/307/316/326/331, 11/23/25/29/37/75/89/157/183/194/198/214/226/230/248/249/250/251/266/268/298/307/316/326/331, 11/23/25/29/37/38/75/89/93/157/183/192/194/198/214/226/230/248/249/250/251/266/268/298/307/316/326/331, 11/23/25/29/37/38/75/89/157/183/192/194/198/214/226/230/248/249/250/251/266/268/298/307/316/326/331, 11/23/25/29/37/75/89/157/183/194/198/214/226/230/249/250/250/251/266/268/298/307/316/326/331, 11/23/25/29/37/75/89/157/183/194/198/214/226/230/248/249/250/251/266/268/298/307/316/326/331/344, 11/23/25/29/37/75/89/157/183/192/194/198/214/226/230/248/249/250/251/266/268/298/307/316/326/331, 11/23/25/29/37/38/75/89/93/157/183/194/198/214/226/230/249/250/251/266/268/277/298/307/316/326/331, 11/23/25/29/37/38/75/89/157/183/194/198/214/226/230/249/250/251/255/266/268/280/298/307/316/318/326/331, 11/23/25/29/37/38/75/86/89/157/183/194/198/214/226/230//241/249/250/251/255/266/268/280/298/307/316/318/326/331/335, 11/23/25/29/37/38/75/89/157/183/194/198/214/226/230/249/250/251/266/268/298/307/316/326/331, 11/23/25/29/37/75/89/157/183/194/198/214/226/230/249/250/251/255/266/268/298/307/316/326/331, 11/23/25/29/37/75/89/157/183/194/198/214/226/230/249/250/251/266/268/298/307/316/318/326/331, 11/23/25/29/37/38/75/89/101/157/183/194/198/214/226/230/249/250/251/255/266/268/280/298/307/316/326/331, 11/23/25/29/37/3875/89/157/183/194/198/214/226/230/249/250/251/266/268/298/307/316/318/326/331/335, 11/23/25/29/37/38/75/89/157/183/194/198/214/226/230/249/250/251/255/266/268/298/307/316/318/326/331, 11/23/25/29/37/75/89/157/183/192/194/198/214/226/230/249/250/251/266/268/298/307/316/326/331/344, 11/23/25/29/37/38/75/89/157/183/194/198/214/226/230/249/250/251/266/268/298/307/316/326/331/335, 11/23/25/29/37/75/89/157/183/194/198/214/226/230/249/250/251/266/268/298/307/316/318/326/331/335, 11/23/25/29/37/38/75/89/157/183/194/198/214/226/230/249/250/251/266/268/277/298/307/316/326/331, 11/23/25/29/37/38/75/89/93/157/183/194/198/214/226/230/249/250/251/266/268/298/307/316/326/331, 11/23/25/29/37/75/89/157/183/192/194/198/214/226/230/249/250/251/266/268/298/307/316/326/331, 11/23/25/29/37/38/64/75/89/157/183/194/198/214/226/230/249/250/251/266/268/298/307/316/318/326/331, 11/23/25/29/37/38/64/75/89/157/183/194/198/214/226/230/241/249/250/251/255/266/268/298/307/316/318/326/331, 11/23/25/29/37/75/89/157/183/194/198/214/226/230/249/250/251/266/268/298/307/316/326/331, 11/23/25/29/37/64/75/89/101/157/183/194/198/214/226/230/249/250/251/255/266/268/280/298/307/316/318/326/331, 11/23/25/29/37/38/75/89/101/157/183/194/198/214/226/230/249/250/251/255/266/268/280/298/307/316/318/326/331/335, 11/23/25/29/37/64/75/89/157/183/194/198/214/226/230/249/250/251/266/268/298/307/316/318/326/331, 11/23/25/29/37/38/64/75/89/157/183/194/198/214/226/230/249/250/251/255/266/268/298/307/316//318/326/331, 11/23/25/29/37/75/89/157/183/194/198/214/226/230/241/249/250/251/255/266/268/280/298/307/316/318/326/331, 11/23/25/29/37/38/75/86/89/101/157/183/194/198/214/226/230/241/249/250/251/266/268/298/307/316/318/326/331, 11/23/25/29/37/38/75/89/157/183/194/198/214/226/230/241/249/250/251/266/268/298/307/316/326/331, 11/23/25/29/37/38/75/89/157/183/194/198/214/226/230/249/250/251/255/266/268/298/307/316/318/326/331/335, 11/23/25/29/37/38/64/75/89/101/157/183/194/198/214/226/230/249/250/251/266/268/298/307/316/318/326/331, 11/23/25/29/37/38/75/64/89/157/183/194/198/214/226/230/249/250/251/255/266/268/280/298/307/316/318/326/331, 11/23/25/29/37/75/89/157/183/194/198/214/226/230/241/249/250/251/255/266/268/298/307/316//318/326/331. 11/23/25/29/37/38/75/89/157/183/194/198/214/226/230/241/249/250/251/266/268/298/307/316/326/331/335, wherein the amino acid positions are relative to the reference sequence corresponding to residues 12 to 346 of SEQ ID NO: 2, or relative to the reference sequence corresponding to SEQ ID NO: 2.

In some embodiments, the amino acid sequence of the engineered RNA ligase comprises at least a substitution set or amino acid residue 11N/23T/25A/29S/37L/38S/75D/89M/157V/183L/194V/198M/214L/226R/230V/248F/249L/250I/251E/266L/268G/298S/307R/316L/326L/331G, 11N/23T/25A/29S/37L/38S/75D/89M/157V/183L/194V/198M/214L/226R/230V/248F/249L//250E/250I/251 E/266L/268G/277A/298S/307R/316L/326L/331G, 11N/23T/25A/29S/37L/75D/89M/157V/183L/194V/198M/214L/226R/230V/248F/249L/250E/251E/266L/268G/298S/307R/316L/326L/331G, 11N/23T/25A/29S/37L/38S/75D/89M/93K/157V/183L/192E/194V/198M/214L/226R/230V/248F/249L/250I/251E/266L/268G/298S/307R/316L/326L/331G, 11N/23T/25A/29S/37L/38S/75D/89M/157V/183L/192E/194V/198M/214L/226R/230V/248F/249L/250I/251E/266L/268G/298S/307R/316L/326L/331G, 11N/23T/25A/29S/37L/75D/89M/157V/183L/194V/198M/214L/226R/230V/249L/250E/250I/251E/266L/268 G/298S/307R/316L/326L/331G, 11N/23T/25A/29S/37L/38S/75D/89M/157V/183L/194V/198M/214L/226R/230V/248F/249L/250E/251E/266 L/268G/298S/307R/316L/326L/331G, 11N/23T/25A/29S/37L/75D/89M/157V/183L/194V/198M/214L/226R/230V/248F/249L/250I/251E/266L/268 G/298S/307R/316L/326L/331G/344K, 11N/23T/25A/29S/37L/75D/89M/157V/183L/192E/194V/198M/214L/226R/230V/248F/249L/250E/251R/266L/268G/298S/307R/316L/326L/331G, 11N/23T/25A/29S/37L/38S/75D/89M/157V/183L/194V/198M/214L/226R/230V/248F/249L/250E/251S/266 L/268G/298S/307R/316L/326L/331G, 11N/23T/25A/29S/37L/38S/75D/89M/93K/157V/183L/194V/198M/214L/226R/230V/249L/250E/251E/266L/268G/277A/298S/307R/316L/326L/331G, 11N/23T/25A/29S/37L/38A/75D/89M/157V/183L/194V/198M/214L/226R/230V/249L/250I/251S/255V/266 L/268G/280N/298S/307R/316L/318S/326L/331G, 11N/23T/25A/29S/37L/75D/89M/157V/183L/194V/198M/214L/226R/230V/248F/249L/250I/251S/266L/268 G/298S/307R/316L/326L/331G, 11N/23T/25A/29S/37L/38A/75D/86D/89M/157V/183L/194V/198M/214L/226R/230V/241G/249L/250I/251S/255V/266L/268G/280N/298S/307R/316L/318S/326L/331G/335Q, 11N/23T/25A/29S/37L/38A/75D/89M/157V/183L/194V/198M/214L/226R/230V/249L/250I/251S/266L/268 G/298S/307R/316L/326L/331G, 11N/23T/25A/29S/37L/75D/89M/157V/183L/194V/198M/214L/226R/230V/249L/250I/251S/255S/266L/268 G/298S/307R/316L/326L/331G, 11N/23T/25A/29S/37L/75D/89M/157V/183L/194V/198M/214L/226R/230V/249L/250I/251S/266L/268G/298S/307R/316L/318S/326L/331G, 11N/23T/25A/29S/37L/38A/75D/89M/101P/157V/183L/194V/198M/214L/226R/230V/249L/250I/251S/255 S/266L/268G/280N/298S/307R/316L/326L/331G, 11N/23T/25A/29S/37L/38A/75D/89M/157V/183L/194V/198M/214L/226R/230V/249L/2501/251S/266L/268 G/298S/307R/316L/318S/326L/331G/335Q, 11N/23T/25A/29S/37L/38A/75D/89M/157V/183L/194V/198M/214L/226R/230V/249L/250I/251S/255V/266 L/268G/298S/307R/316L/318S/326L/331G, 11N/23T/25A/29S/37L/75D/89M/157V/183L/192E/194V/198M/214L/226R/230V/249L/250E/251E/266L/268G/298S/307R/316L/326L/331G/344K, 11N/23T/25A/29S/37L/38A/75D/89M/157V/183L/194V/198M/214L/226R/230V/249L/250I/251S/266L/268 G/298S/307R/316L/326L/331G/335Q, 11N/23T/25A/29S/37L/75D/89M/157V/183L/194V/198M/214L/226R/230V/249L/2501/251S/266L/268G/298S/307R/316L/318S/326L/331G/335Q, 11N/23T/25A/29S/37L/38S/75D/89M/157V/183L/194V/198M/214L/226R/230V/249L/250I/251S/266L/268 G/277A/298S/307R/316L/326L/331G, 11N/23T/25A/29S/37L/38S/75D/89M/93K/157V/183L/194V/198M/214L/226R/230V/249L/250I/251E/266L/268G/298S/307R/316L/326L/331G, 11N/23T/25A/29S/37L/75D/89M/157V/183L/192E/194V/198M/214L/226R/230V/249L/250I/251E/266L/268 G/298S/307R/316L/326L/331G, 11N/23T/25A/29S/37L/75D/89M/157V/183L/194V/198M/214L/226R/230V/249L/250I/251S/255S/266L/268 G/298S/307R/316L/326L/331G, 11N/23T/25A/29S/37L/38A/64Q/75D/89M/157V/183L/194V/198M/214L/226R/230V/249L/2501/251S/266L/268G/298S/307R/316L/318S/326L/331G, 11N/23T/25A/29S/37L/75D/89M/157V/183L/194V/198M/214L/226R/230V/248I/249L/250I/251S/266L/268 G/298S/307R/316L/326L/331G, 11N/23T/25A/29S/37L/38A/64Q/75D/89M/157V/183L/194V/198M/214L/226R/230V/241G/249L/2501/251S/255V/266L/268G/298S/307R/316L/318S/326L/331G, 11N/23T/25A/29S/37L/75D/89M/157V/183L/194V/198M/214L/226R/230V/249L/250I/251E/266L/268G/298S/307R/316L/326L/331G, 11N/23T/25A/29S/37L/64Q/75D/89M/101P/157V/183L/194V/198M/214L/226R/230V/249L/250I/251S/255 V/266L/268G/280N/298S/307R/316L/318S/326L/331G, 11N/23T/25A/29S/37L/38A/75D/89M/101P/157V/183L/194V/198M/214L/226R/230V/249L/250I/251S/255 V/266L/268G/280N/298S/307R/316L/318S/326L/331G/335Q, 11N/23T/25A/29S/37L/75D/89M/157V/183L/194V/198M/214L/226R/230V/249L/250I/251S/266L/268G/298S/307R/316L/326L331G, 11N/23T/25A/29S/37L/38S/75D/89M/157V/183L/194V/198M/214L/226R/230V/249L/250I/251R/266L/268 G/277A/298S/307R/316L/326L/331G, 11N/23T/25A/29S/37L/64Q/75D/89M/157V/183L/194V/198M/214L/226R/230V/249L/250I/251S/266L/268 G/298S/307R/316L/318S/326L/331G, 11N/23T/25A/29S/37L/38A/64Q/75D/89M/157V/183L/194V/198M/214L/226R/230V/249L/250I/251S/255V/266L/268G/298S/307R/316L/318S/326L/331G, 11N/23T/25A/29S/37L/75D/89M/157V/183L/194V/198M/214L/226R/230V/241G/249L/250I/251S/255S/266 L/268G/280N/298S/307R/316L/318S/326L/331G, 11N/23T/25A/29S/37L/75D/89M/157V/183L/194V/198M/214L/226R/230V/249L/2501/251S/255V/266L/268G/298S/307R/316L/326L/331G, 11N/23T/25A/29S/37L/38A/75D/86D/89M/101P/157V/183L/194V/198M/214L/226R/230V/241G/249L/250I/251S/266L/268G/298S/307R/316L/318S/326L/331G, 11N/23T/25A/29S/37L/38A/75D/89M/157V/183L/194V/198M/214L/226R/230V/241G/249L/250I/251S/266 L/268G/298S/307R/316L/326L/331G, 11N/23T/25A/29S/37L/38A/75D/89M/157V/183L/194V/198M/214L/226R/230V/249L/250I/251S/255S/266 L/268G/298S/307R/316L/318S/326L/331G/335Q, 11N/23T/25A/29S/37L/38A/64Q/75D/89M/101P/157V/183L/194V/198M/214L/226R/230V/249L/250I/251S/266L/268G/298S/307R/316L/318S/326L/331G, 11N/23T/25A/29S/37L/38A/75D/64Q/89M/157V/183L/194V/198M/214L/226R/230V/249L/250I/251S/255V/266L/268G/280N/298S/307R/316L/318S/326L/331G, 11N/23T/25A/29S/37L/75D/89M/157V/183L/194V/198M/214L/226R/230V/241G/249L/250I/251S/255S/266 L/268G/298S/307R/316L//318S/326L/331G, 11N/23T/25A/29S/37L/38A/75D/89M/157V/183L/194V/198M/214L/226R/230V/241G/249L/250I/251S/266 L/268G/298S/307R/316L/326L/331G/335Q, wherein the amino acid positions are relative to the reference sequence corresponding to residues 12 to 346 of SEQ ID NO: 2, or relative to the reference sequence corresponding to SEQ ID NO: 2.

In some embodiments, the amino acid sequence of the engineered RNA ligase comprises at least a substitution at an amino acid position provided in Tables 5.1, 6.1, 6.2, 7.1, 8.1, 8.2, 9.1, 10.1, 11.1, 12.1, 13.1, 14.1, 15.1, 16.1, 17.1, 18.1, 19.1, 20.1, 21.1, 22.1, 23.1, 24.1, 25.1, and 26.1, wherein the amino acid positions are relative to the reference sequence corresponding to residues 12 to 346 of SEQ ID NO: 2, or relative to the reference sequence corresponding to SEQ ID NO: 2.

In some embodiments, the amino acid sequence of the engineered RNA ligase comprises at least one substitution provided in Tables 5.1, 6.1, 6.2, 7.1, 8.1, 8.2, 9.1, 10.1, 11.1, 12.1, 13.1, 14.1, 15.1, 16.1, 17.1, 18.1, 19.1, 20.1, 21.1, 22.1, 23.1, 24.1, 25.1, and 26.1, wherein the amino acid positions are relative to the reference sequence corresponding to residues 12 to 346 of SEQ ID NO: 2, or relative to the reference sequence corresponding to SEQ ID NO: 2.

In some embodiments, the amino acid sequence of the engineered RNA ligase comprises at least a substitution or substitution set at the amino acid position(s) provided in Tables 5.1, 6.1, 6.2, 7.1, 8.1, 8.2, 9.1, 10.1, 11.1, 12.1, 13.1, 14.1, 15.1, 16.1, 17.1, 18.1, 19.1, 20.1, 21.1, 22.1, 23.1, 24.1, 25.1, and 26.1, wherein the amino acid positions are relative to the reference sequence corresponding to residues 12 to 346 of SEQ ID NO: 2, or relative to the reference sequence corresponding to SEQ ID NO: 2.

In some embodiments, the amino acid sequence of the engineered RNA ligase comprises at least a substitution or substitution set of an RNA ligase variant provided in Tables 5.1, 6.1, 6.2, 7.1, 8.1, 8.2, 9.1, 10.1, 11.1, 12.1, 13.1, 14.1, 15.1, 16.1, 17.1, 18.1, 19.1, 20.1, 21.1, 22.1, 23.1, 24.1, 25.1, and 26.1, wherein the amino acid positions are relative to the reference sequence corresponding to residues 12 to 346 of SEQ ID NO: 2, or relative to the reference sequence corresponding to SEQ ID NO: 2.

In some embodiments, the engineered RNA ligase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to a reference sequence comprising a substitution or substitution set of an RNA ligase variant provided in Tables 5.1, 6.1, 6.2, 7.1, 8.1, 8.2, 9.1, 10.1, 11.1, 12.1, 13.1, 14.1, 15.1, 16.1, 17.1, 18.1, 19.1, 20.1, 21.1, 22.1, 23.1, 24.1, 25.1, and 26.1, wherein the amino acid positions are relative to the reference sequence corresponding to residues 12 to 346 of SEQ ID NO: 2, or relative to the reference sequence corresponding to SEQ ID NO: 2.

In some embodiments, the engineered RNA ligase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12 to 346 of SEQ ID NO: 54, 244, 648, 862, 956, 1088, 1416, 1612, 1840, 1866, 1954, 2062, 2674, 2754, or 2786, or to the reference sequence corresponding to SEQ ID NO: 54, 244, 648, 862, 956, 1088, 1416, 1612, 1840, 1866, 1954, 2062, 2674, 2754, or 2786.

In some embodiments, the engineered RNA ligase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12 to 346 of an even-numbered SEQ ID NO. of SEQ ID NOs: 4-1470 and 1478-3058, or to the reference sequence corresponding to an even-numbered SEQ ID NO. of SEQ ID NOs: 4-1470 and 1478-3058.

In some embodiments, the engineered RNA ligase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12 to 346 of SEQ ID NO: 54, 244, 648, 862, 956, 1088, 1416, 1612, 1840, 1866, 1954, 2062, 2674, 2754, or 2786, or to the reference sequence corresponding to SEQ ID NO: 54, 244, 648, 862, 956, 1088, 1416, 1612, 1840, 1866, 1954, 2062, 2674, 2754, or 2786, wherein the amino acid sequence comprises one or more substitutions relative to the reference sequence corresponding to residues 12 to 346 of SEQ ID NO: 54, 244, 648, 862, 956, 1088, 1416, 1612, 1840, 1866, 1954, 2062, 2674, 2754, or 2786, or relative to the reference sequence corresponding to SEQ ID NO: 54, 244, 648, 862, 956, 1088, 1416, 1612, 1840, 1866, 1954, 2062, 2674, 2754, or 2786.

In some embodiments, the engineered RNA ligase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12 to 346 of an even-numbered SEQ ID NO. of SEQ ID NOs: 4-1470 and 1478-3058, or to the reference sequence corresponding to an even-numbered SEQ ID NO. of SEQ ID NOs: 4-1470 and 1478-3058, wherein the amino acid sequence comprises one or more substitutions relative to the reference sequence corresponding to residues 12 to 346 of SEQ ID NO: 54, 244, 648, 862, 956, 1088, 1416, 1612, 1840, 1866, 1954, 2062, 2674, 2754, or 2786, or relative to the reference sequence corresponding to SEQ ID NO: 54, 244, 648, 862, 956, 1088, 1416, 1612, 1840, 1866, 1954, 2062, 2674, 2754, or 2786.

In some embodiments, the amino acid sequence of the engineered RNA ligase comprises at least a substitution at amino acid position 10, 11, 12, 13, 15, 17, 18, 21, 23, 25, 26, 28, 29, 30, 32, 33, 37, 38, 39, 40, 41, 42, 45, 52, 54, 56, 57, 58, 60, 61, 62, 64, 65, 67, 71, 73, 74, 75, 77, 80, 81, 83, 84, 86, 87, 89, 90, 91, 93, 95, 97, 99, 100, 101, 102, 103, 104, 105, 106, 107, 109, 113, 114, 116, 117, 119, 121, 122, 123, 125, 126, 127, 128, 129, 131, 135, 137, 140, 141, 143, 144, 148, 150, 154, 156, 157, 158, 159, 160, 161, 162, 165, 169, 170, 171, 173, 175, 177, 178, 179, 181, 183, 184, 185, 190, 191, 192, 193, 194, 197, 198, 204, 206, 208, 210, 212, 213, 214, 215, 218, 223, 224, 226, 228, 229, 230, 232, 236, 238, 241, 243, 247, 248, 249, 250, 251, 252, 253, 254, 255, 256, 257, 258, 260, 261, 264, 265, 266, 267, 268, 270, 271, 272, 274, 276, 277, 280, 282, 283, 286, 288, 289, 290, 291, 292, 294, 295, 296, 297, 298, 299, 300, 301, 302, 305, 306, 307, 310, 311, 315, 316, 317, 318, 323, 324, 325, 326, 327, 330, 331, 332, 334, 335, 336, 337, 338, 341, 342, 344, 345, or 346, or any combinations thereof, wherein the amino acid positions are relative to the reference sequence corresponding to residues 12 to 346 of SEQ ID NO: 54, 244, 648, 862, 956, 1088, 1416, 1612, 1840, 1866, 1954, 2062, 2674, 2754, or 2786, or relative to the reference sequence corresponding to SEQ ID NO: 54, 244, 648, 862, 956, 1088, 1416, 1612, 1840, 1866, 1954, 2062, 2674, 2754, or 2786.

In some embodiments, the amino acid sequence of the engineered RNA ligase comprises at least a substitution or an amino acid residue 10F, 11G/N, 12A/F/L/M/P/R/T/V, 13W, 15M, 17P, 18A/H, 21S, 23K/T, 25A/D/F/G/L/M/S/T, 26L, 28A/L/M/V, 29D/S, 30A/L/T/V/W, 32E, 33A/I/L/T/V, 37A/F/K/L/M/R, 38A/E/L/R/S/T/V, 39P, 40D/I/P/S, 41E/I, 42T, 45S, 52C, 54L, 56I/L, 57V, 58M, 60A/C/D/R/S/V, 61A, 62A/V, 64A/Q/W, 65C, 67R, 71T, 73Q/W, 74D, 75A/D, 77M, 80E, 81Y, 83A/E/G/L/T/V, 84V, 86D/G/L, 87E, 89C/D/E/G/L/M/N/Q/R/S/T/V/W/Y, 90E/F, 91A/E/V, 93K/V, 95A/C/G/I, 97A/E, 99I, 100A/G/H/L/R/S/T/V, 101P/V/W, 102T, 103G/V, 104T, 105K/L/R, 106G/L/M/Q/R/V/W, 107M/S, 109F/L, 113L/M, 114E, 116L, 117A/Q, 119L, 121N, 122C/F/G/L/Q/T/W, 123K/R, 125K/T, 126M, 127P, 128E, 129L/W, 131T, 135A/H/M/Q/V, 137N/R, 140I, 141D/G, 143A/C/E/F/H/M/S/T/V, 144L, 148F, 150D/L, 154S/V, 156G/H/Q/S, 157F/L/Q/V, 158R/S, 159F, 160R, 161M/V, 162C, 165M, 169G/N, 170H/Y, 171S, 173M, 175A, 177K, 178V, 179E, 181E/N/P/R/S, 183L/V, 184L, 185C/F/G/K/M/P/S/T/V, 190I/M, 191C, 192D/E/F/L/N, 193K/Q, 194A/G/V, 197W, 198M, 204W, 206L, 208H/I/K/L/P/T, 210G/V, 212E/G/I/K/L, 213D/N/R/T, 214A/C/H/K/L/Q/Y, 215G/T, 218F, 223C, 224A/Q/R, 226R, 228D/L, 229W, 230T/V, 232G/M/N/R/S, 236L/V, 238K/S/V, 241E/G/Q/R, 243H, 247I/L/R/S, 248F/G/I/K/T, 249K/L/R/S, 250E/I/S, 251C/E/F/G/L/R/S/T/Y, 252A/Q, 253L/W, 254E/L/N/Q/S/V, 255N/S/V/Y, 256A/E/G/M/S/W, 257D/E/G, 258D/M/P/V, 260G, 261D/P/T, 264A/E/G/S/V, 265A/F, 266C/I/L, 267I/M, 268F/G/M, 270L/M/V, 271A/E/G/T, 272M/S, 274A, 276A/G/M/V, 277A, 280L/N/W, 282A/M, 283C/V, 286D/E/F/M/V, 288E/L/R/W, 289A/D/E/I/P/T, 290D/H/N/P, 291H/L/Q/R/S/T/W, 292N/S, 294A/S/V/Y, 295C/F/K/L/M/P/Q/R/S/T/V/Y, 296E/G/L/Q/W, 297A/I/L, 298C/I/K/R/S/V, 299E/I/K/Q/R/T/V, 300A, 301A/F/I/L/M/V, 302A/C/I/L/S, 305V, 306G/L/T, 307K/R, 310H/L/M/N/Q/R/S/T, 311C, 315E/S, 316I/L/Y, 317E/F/G/Q/V/W, 318A/E/G/S, 323E/G, 324D/G/M, 325L, 326C/L, 327E/G/M/S, 330A/D/E/G/K/R, 331D/G/R/S/W, 332E/H/W, 334E/R, 335H/Q, 336I/L/M, 337A/V, 338G/L/M, 341A, 342R/S/T, 344K, 345Q/S, or 346S, or any combinations thereof, wherein the amino acid positions are relative to the reference sequence corresponding to residues 12 to 346 of SEQ ID NO: 54, 244, 648, 862, 956, 1088, 1416, 1612, 1840, 1866, 1954, 2062, 2674, 2754, or 2786, or relative to the reference sequence corresponding to SEQ ID NO: 54, 244, 648, 862, 956, 1088, 1416, 1612, 1840, 1866, 1954, 2062, 2674, 2754, or 2786.

In some embodiments, the amino acid sequence of the engineered RNA ligase comprises at least a substitution at amino acid position 10, 11, 12, 13, 17, 21, 23, 25, 26, 28, 29, 30, 32, 37, 38, 40, 41, 42, 45, 52, 54, 56, 57, 58, 60, 61, 62, 64, 65, 67, 71, 73, 74, 75, 77, 80, 83, 84, 87, 90, 91, 93, 97, 99, 100, 101, 102, 103, 104, 105, 106, 107, 109, 113, 114, 121, 122, 123, 125, 126, 127, 128, 129, 131, 135, 137, 140, 141, 143, 148, 150, 157, 158, 160, 161, 162, 165, 169, 170, 173, 178, 179, 184, 190, 191, 192, 193, 194, 197, 198, 204, 206, 208, 210, 213, 215, 218, 223, 228, 229, 232, 236, 238, 241, 243, 248, 249, 250, 251, 252, 253, 254, 255, 258, 261, 264, 265, 267, 268, 270, 271, 272, 274, 276, 277, 282, 286, 289, 290, 292, 294, 295, 297, 298, 299, 300, 301, 302, 305, 311, 318, 323, 324, 325, 327, 332, 336, 338, 341, 344, or 346; a substitution or amino acid residue 18A/H, 33A/I/T/V, 39P, 86D/L, 89C/D/E/L/N/Q/R/S/T/V/Y, 95A/C/G/I, 116L, 117A/Q, 119L, 144L, 154S/V, 156G/H/S, 171S, 175A, 177K, 181E/N/P/R/S, 183L, 212E/G/K/L, 224A/Q/R, 226R, 230T/V, 247I/L/R/S, 256E/G/M/S/W, 257D/E/G, 260G, 280L/W, 283C/V, 288L/R/W, 291S, 296E/G/L/Q/W, 306G/T, 307K/R, 310H/L/M/N/Q/R/S/T, 315E, 316I/Y, 317E/F/G/Q/V/W, 326C/L, 330A/D/E/G/K, 331D/G/S, 334E, 335Q, 337A/V, 338G/L/M, 342R/S/T, or 345Q/S; or any combinations thereof, wherein the amino acid positions are relative to the reference sequence corresponding to residues 12 to 346 of SEQ ID NO: 54, 244, 648, 862, 956, 1088, 1416, 1612, 1840, 1866, 1954, 2062, 2674, 2754, or 2786, or relative to the reference sequence corresponding to SEQ ID NO: 54, 244, 648, 862, 956, 1088, 1416, 1612, 1840, 1866, 1954, 2062, 2674, 2754, or 2786.

In some embodiments, the amino acid sequence of the engineered RNA ligase comprises at least a substitution or amino acid residue 10F, 11G/N, 12A/F/L/M/P/R/T/V, 13W, 17P, 18A/H, 21S, 23K/T, 25A/D/F/G/L/M/S/T, 26L, 28A/L/M/V, 29D/S, 30A/L/T/V/W, 32E, 33A/I/T/V, 37A/F/K/L/M/R, 38A/E/L/R/S/T/V, 39P, 40D/I/P/S, 41E/I, 42T, 45S, 52C, 54L, 56I/L, 57V, 58M, 60A/C/D/R/S/V, 61A, 62A/V, 64A/Q/W, 65C, 67R, 71T, 73Q/W, 74D, 75A/D, 77M, 80E, 83A/E/G/L/T/V, 84V, 86D/L, 87E, 89C/D/E/L/N/Q/R/S/T/V/Y,90E/F,91A/E/V, 93K/V, 95A/C/G/I, 97A/E, 991, 100A/G/H/L/R/S/T/V, 101P/V/W, 102T, 103G/V, 104T, 105K/L/R, 106G/L/M/Q/R/V/W, 107M/S, 109F/L, 113L/M, 114E, 116L, 117A/Q, 119L, 121N, 122C/F/G/L/Q/T/W, 123K/R, 125K/T, 126M, 127P, 128E, 129L/W, 131T, 135A/H/M/Q/V, 137N/R, 140I, 141D/G, 143A/C/E/F/H/M/S/T/V, 144L, 148F, 150D/L, 154S/V, 156G/H/S, 157F/L/Q/V, 158R/S, 160R, 161M/V, 162C, 165M, 169G/N, 170H/Y, 171S, 173M, 175A, 177K, 178V, 179E, 181E/N/P/R/S, 183L, 184L, 185C/F/M/P/S/T/V, 190I/M, 191C, 192D/E/F/L/N, 193K/Q, 194A/G/V, 197W, 198M, 204W, 206L, 208H/I/K/L/P/T, 210G/V, 212E/G/K/L, 213D/N/R/T, 214A/C/H/K/Q/Y, 215G/T, 218F, 223C, 224A/Q/R, 226R, 228D/L, 229W, 230T/V, 232G/M/N/R/S, 236L/V, 238K/S/V, 241E/G/Q/R, 243H, 247I/L/R/S, 248F/G/I/K/T, 249K/L/R/S, 250E/I/S, 251C/E/F/G/L/R/S/T/Y, 252A/Q, 253L/W, 254E/K/L/N/Q/S/V,255N/S/V/Y, 256E/G/M/S/W, 257D/E/G, 258D/M/P/V, 260G, 261D/P/T, 264A/E/G/S/V, 265A/F, 266C/I, 267I/M, 268F/G/M, 270L/M/V, 271A/E/G/T, 272M/S, 274A, 276A/G/M/V, 277A, 280L/W, 282A/M, 283C/V, 286D/E/F/M/V, 288L/R/W, 289A/D/E/I/P/T, 290D/H/N/P, 291H/L/Q/R/S/T/W, 291S, 292N/S, 294A/S/V/Y, 295C/F/K/L/M/P/Q/R/S/T/V/Y, 295G, 296E/G/L/Q/W, 297A/I/L, 298I/S, 298C/K/R/V, 299E/I/K/Q/R/T/V, 300A, 301A/F/I/L/M/V, 302A/C/I/L/S, 305V, 306G/T, 307K/R, 310H/L/M/N/Q/R/S/T, 311C, 315E, 316I/Y, 317E/F/G/Q/V/W, 318A/E/G/S, 323E/G, 324D/G/M, 325L, 326C/L, 327E/G/M/S, 330A/D/E/G/K, 331D/G/S, 332E/H/W, 334E, 335Q, 336I/L/M, 337A/V, 338G/L/M, 341A, 342R/S/T, 344K, 345Q/S, 346S, or any combinations thereof, wherein the amino acid positions are relative to the reference sequence corresponding to residues 12 to 346 of SEQ ID NO: 54, 244, 648, 862, 956, 1088, 1416, 1612, 1840, 1866, 1954, 2062, 2674, 2754, or 2786, or relative to the reference sequence corresponding to SEQ ID NO: 54, 244, 648, 862, 956, 1088, 1416, 1612, 1840, 1866, 1954, 2062, 2674, 2754, or 2786.

In some embodiments, the amino acid sequence of the engineered RNA ligase comprises at least a substitution at amino acid position 11, 12, 23, 25, 29, 37, 38, 75, 86, 89, 93, 101, 122, 143, 157, 183, 192, 194, 198, 212, 214, 230, 241, 248, 249, 250, 251, 254, 266, 268, 280, 291, 295, 298, 307, 316, 318, 326, 330, or 331, or any combinations thereof, wherein the amino acid positions are relative to the reference sequence corresponding to residues 12 to 346 of SEQ ID NO: 54, 244, 648, 862, 956, 1088, 1416, 1612, 1840, 1866, 1954, 2062, 2674, 2754, or 2786, or relative to the reference sequence corresponding to SEQ ID NO: 54, 244, 648, 862, 956, 1088, 1416, 1612, 1840, 1866, 1954, 2062, 2674, 2754, or 2786.

In some embodiments, the amino acid sequence of the engineered RNA ligase comprises at least a substitution or an amino acid residue 11G/N, 12A/F/L/M/P/R/T/V, 23K/T, 25A/D/F/G/L/M/S/T, 29D/S, 37A/F/K/L/M/R, 38A/E/L/R/S/T/V, 75A/D, 86D/G/L, 89C/D/E/G/L/M/N/Q/R/S/T/V/W/Y, 93K/V, 101P/V/W, 122C/F/G/L/Q/T/W, 143A/C/E/F/H/M/S/T/V, 157F/L/Q/V, 183L/V, 192D/E/F/L/N, 194A/G/V, 198M, 212E/G/I/K/L, 214A/C/H/K/L/Q/Y, 230T/V, 241E/G/Q/R, 248F/G/I/K/T, 249K/L/R/S, 250E/I/S, 251C/E/F/G/L/R/S/T/Y, 254E/L/N/Q/S/V, 266C/I/L, 268F/G/M, 280L/N/W, 291H/L/Q/R/S/T/W, 295C/F/K/L/M/P/Q/R/S/T/V/Y, 298C/I/K/R/S/V, 307K/R, 316I/L/Y, 318A/E/G/S, 326C/L, 330A/D/E/G/K/R, or 331D/G/R/S/W, or any combinations thereof, wherein the amino acid positions are relative to the reference sequence corresponding to residues 12 to 346 of SEQ ID NO: 54, 244, 648, 862, 956, 1088, 1416, 1612, 1840, 1866, 1954, 2062, 2674, 2754, or 2786, or relative to the reference sequence corresponding to SEQ ID NO: 54, 244, 648, 862, 956, 1088, 1416, 1612, 1840, 1866, 1954, 2062, 2674, 2754, or 2786.

In some embodiments, the amino acid sequence of the engineered RNA ligase comprises at least a substitution or an amino acid residue 11N, 12M/T, 23T, 25A, 29S, 37L, 38A/S, 75D, 86D, 89M, 93K, 101P, 122F, 143S, 157V, 183L, 192D/E, 194V, 198M, 212E, 214L, 226R, 230T/V, 241G, 248F, 249L, 250I, 251E/S, 254K/S, 266L, 268G, 280L, 291Q, 295G/V, 298S/V, 307R, 316L, 318S, 326L, 330A, or 331G, or any combinations thereof, wherein the amino acid positions are relative to the reference sequence corresponding to residues 12 to 346 of SEQ ID NO: 54, 244, 648, 862, 956, 1088, 1416, 1612, 1840, 1866, 1954, 2062, 2674, 2754, or 2786, or relative to the reference sequence corresponding to SEQ ID NO: 54, 244, 648, 862, 956, 1088, 1416, 1612, 1840, 1866, 1954, 2062, 2674, 2754, or 2786.

In some embodiments, the amino acid sequence of the engineered RNA ligase comprises at least a substitution at amino acid position 11, 12, 23, 25, 29, 37, 38, 75, 93, 101, 122, 143, 157, 192, 194, 198, 241, 248, 249, 250, 251, 254, 268, 295, 298, or 318; a substitution 86D/L, 89C/D/E/L/N/Q/R/S/T/V/Y, 183L, 212E/G/K/L, 214A/C/H/K/L/Q/Y, 226R, 230T/V, 266C/I, 280L/W, 291H/L/Q/R/S/T/W, 307K/R, 326C/L, 330A/D/E/G/K, or 331D/G/S; or any combinations thereof, wherein the amino acid positions are relative to the reference sequence corresponding to residues 12 to 346 of SEQ ID NO: 54, 244, 648, 862, 956, 1088, 1416, 1612, 1840, 1866, 1954, 2062, 2674, 2754, or 2786, or relative to the reference sequence corresponding to SEQ ID NO: 54, 244, 648, 862, 956, 1088, 1416, 1612, 1840, 1866, 1954, 2062, 2674, 2754, or 2786.

In some embodiments, the amino acid sequence of the engineered RNA ligase comprises at least a substitution or amino acid residue 11G/N, 12A/F/L/M/P/R/T/V, 23K/T, 25A/D/F/G/L/M/S/T, 29D/S, 37A/F/K/L/M/R, 38A/E/L/R/S/T/V, 75A/D, 86D/L, 89C/D/E/L/N/Q/R/S/T/V/Y, 93K/V, 101P/V/W, 122C/F/G/L/Q/T/W, 143A/C/E/F/H/M/S/T/V, 157F/L/Q/V, 183L, 192D/E/F/L/N, 194A/G/V, 198M, 212E/G/K/L, 241E/G/Q/R, 214A/C/H/K/L/Q/Y, 226R, 230T/V, 248F/G/I/K/T, 249K/L/R/S, 250E/I/S, 251C/E/F/G/L/R/S/T/Y, 254E/L/N/Q/S/V, 266C/I, 268F/G/M, 280L/W, 291H/L/Q/R/S/T/W, 295C/F/K/L/M/P/Q/R/S/T/V/Y, 298C/I/K/R/S/V, 299E/I/K/Q/R/T/V, 307K/R, 318A/E/G/S, 326C/L, 330A/D/E/G/K, or 331D/G/S, or any combinations thereof, wherein the amino acid positions are relative to the reference sequence corresponding to residues 12 to 346 of SEQ ID NO: 54, 244, 648, 862, 956, 1088, 1416, 1612, 1840, 1866, 1954, 2062, 2674, 2754, or 2786, or relative to the reference sequence corresponding to SEQ ID NO: 54, 244, 648, 862, 956, 1088, 1416, 1612, 1840, 1866, 1954, 2062, 2674, 2754, or 2786.

In some embodiments, the amino acid sequence of the engineered RNA ligase comprises at least a substitution or amino acid residue 11N, 12T, 23T, 25A, 29S, 37L, 38A/S, 75D, 86D, 93K, 101P, 122F, 157V, 183L, 192E, 194V, 198M, 212E, 214L, 226R, 230T/V, 241G, 249L, 250I, 251E/S, 254S, 266L, 268G, 280L, 291Q, 295V, 298S/V, 307R, 318S, 326L, 330A, or 331G, or any combinations thereof, wherein the amino acid positions are relative to the reference sequence corresponding to residues 12 to 346 of SEQ ID NO: 54, 244, 648, 862, 956, 1088, 1416, 1612, 1840, 1866, 1954, 2062, 2674, 2754, or 2786, or relative to the reference sequence corresponding to SEQ ID NO: 54, 244, 648, 862, 956, 1088, 1416, 1612, 1840, 1866, 1954, 2062, 2674, 2754, or 2786.

In some embodiments, the amino acid sequence of the engineered RNA ligase further comprises at least a substitution at amino acid position 2, 14, 15, 63, 69, 81, 85, 95, 138, 142, 149, 159, 171, 186, 195, 202, 266, 275, 283, 285, 303, 314, 333, or 339; a substitution 18I, 33L, 39Q/S, 86G, 89G/H/I/M/W, 116N, 117D, 119T, 144W, 154C, 156A/L/N/Q/T, 175K, 177P, 181D, 183V, 185G/K, 2121, 2141, 224Y, 2261, 230D, 247K, 256A, 257L, 260S, 280G/N, 288E, 291P, 296K, 306L, 307E/Q, 310K, 315S/T, 316L, 317A, 326R, 330M/R, 331L/R/W, 334R, 335D/H, 337L, 342I, or 345V; or any combinations thereof, wherein the amino acid positions are relative to the reference sequence corresponding to residues 12 to 346 of SEQ ID NO: 54, 244, 648, 862, 956, 1088, 1416, 1612, 1840, 1866, 1954, 2062, 2674, 2754, or 2786, or relative to the reference sequence corresponding to SEQ ID NO: 54, 244, 648, 862, 956, 1088, 1416, 1612, 1840, 1866, 1954, 2062, 2674, 2754, or 2786.

In some embodiments, the amino acid sequence of the engineered RNA ligase further comprises at least a substitution or amino acid residue 2E/G/V, 14K, 15E/L/M/P/Y, 18I, 33L, 39Q/S, 63V, 69D, 81Y, 85L, 86G, 89G/H/I/M/W, 95V, 116N, 117D, 119T, 138R, 142R, 144W, 149R/T, 154C, 156A/L/N/Q/T, 159F, 171E, 175K, 177P, 181D, 183V, 185G/K, 186D/R, 195G, 202Y, 2121, 214I, 224Y, 226I, 230D, 247K, 256A, 257L, 260S, 266L, 275N, 280G/N, 2831, 285K, 288E, 291P, 296K, 303Q, 306L, 307E/Q, 310K, 314W, 315S/T, 316L, 317A, 326R, 330M/R, 331L/R/W, 333V, 334R, 335D/H, 337L, 339P, 342I, or 345V, or any combinations thereof, wherein the amino acid positions are relative to the reference sequence corresponding to residues 12 to 346 of SEQ ID NO: 54, 244, 648, 862, 956, 1088, 1416, 1612, 1840, 1866, 1954, 2062, 2674, 2754, or 2786, or relative to the reference sequence corresponding to SEQ ID NO: 54, 244, 648, 862, 956, 1088, 1416, 1612, 1840, 1866, 1954, 2062, 2674, 2754, or 2786.

In some embodiments, the engineered RNA ligase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12 to 346 of SEQ ID NO: 54, or to the reference sequence corresponding to SEQ ID NO: 54, wherein the amino acid sequence comprises one or more substitutions relative to the reference sequence corresponding to residues 12 to 346 of SEQ ID NO: 54, or relative to the reference sequence corresponding to SEQ ID NO: 54.

In some embodiments, the engineered RNA ligase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12 to 346 of an even-numbered SEQ ID NO. of SEQ ID NOs: 220-630, or to the reference sequence corresponding to an even-numbered SEQ ID NO. of SEQ ID NOs: 220-630, wherein the amino acid sequence comprises one or more substitutions relative to the reference sequence corresponding to residues 12 to 346 of SEQ ID NO: 54, or relative to the reference sequence corresponding to SEQ ID NO: 54.

In some embodiments, the amino acid sequence of the engineered RNA ligases comprises a substitution or substitution set at amino acid positions(s) 156/232/334/335, 12/254/256, 12/230, 12/230/251, 12/230/251/254/256, 12/38/230, 230/251, 12/38/254, 230/256, 254, 230, 12/230/254, 12/38/230/251/256, 12/251, 12/251/254/256, 38, 12/230/256, 12, 12/25/38/230/251, 12/38/102/230/251/256, 12/256, 38/230/256, 256, 12/230/251/256, 230/251/254, 230/254, 251/254, 12/25/230, 38/230, 38/251, 12/38/230/256, 102, 251, 12/25/251/256, 12/102/256, 12/38/230/251/254/256, 12/251/256, 102/230/251/254/256, 12/38/251, 12/230/251/254, 156/248/334/335, 12/25/38, 12/102/230/251/254, 12/38/251/254, 12/251/254, 12/102, 38/230/251/254/256, 12/25/230/251/256, 12/230/311, 12/254, 38/230/251/254, 12/38/251/256, 156/334/335, 257/258/334, 12/25/38/251/256, 334, 12/25/230/254/256, 38/251/256, 12/38/230/251/254, 12/38, 257/258/334/335, 12/38/254/256, 232/248/257/258/334/335, 156/258/334/335, 156/249/334/335, 12/102/230/254, 25, 156/257/258/334/335, 12/102/230, 156/248/257/258/334, 248/249/334/335, 232/257/258/334/335, 334/335, 156/334, 12/38/102/256, 156/257/334/335, 12/140/251/254, 251/254/256, 248/334/335, 156/258/334, 156/248/330/335, 12/38/256, 232/334/335, 156/249/334, 330/334/335, 156/248/334, 156/335, 257/334, 248/334, 232/248/334/335, 12/25/38/230/251/256, 156/248/249/334/335, 330/335, 248/257/334/335, 232/248/334, 156/232/248/334/335, 232/257/330/334, 156/248/291/334/335, 156/232/248/257/258/334, 156/232/249/334/335, 12/25, 156/248/330/334/335, 156/248/249/334, 232/248/330/335, 156/232/330/334/335, 197, 338, 327, 192, 331, 326, 345, 101, 100, 141, 198, 64, 306, 317, 265, 296, 194, 62, 90, 307, 103, 210, 105, or 61, wherein the amino acid positions are relative to the reference sequence corresponding to residues 12 to 346 of SEQ ID NO: 54, or relative to the reference sequence corresponding to SEQ ID NO: 54.

In some embodiments, the amino acid sequence of the engineered RNA ligases comprises a substitution or substitution set, or amino acid residue(s) 156Q/232G/334R/335H, 12V/254V/256A, 12V/230T, 12L/230T/251T, 12L/230T/251S/254V/256A, 12A/38R/230T, 230T/251S, 12P/38R/254V, 12V/230T/251T, 230T/256A, 254V, 230T, 12T/230T/251S, 12T/230T/254V, 12V/38R/230T/251T/256A, 12L/251S, 12T/251T/254V/256A, 12V/230T/251S, 38R, 12T/230T/256A, 12L, 12V, 12L/25M/38R/230T/251T, 12L/38R/102T/230T/251T/256A, 12L/230T, 12V/256A, 38R/230T/256A, 12V/230T/251S/254V/256A, 256A, 12T/230T/251S/256A, 12T/251T, 230T/251S/254L, 230T/254V, 251S/254L, 12T/25L/230T, 12V/230T/251S/254L/256A, 38R/230T, 12T/251S, 38R/251T, 12V/230T/251S/256A, 12L/230T/251T/254V/256A, 12T/38R/230T/256A, 12L/230T/256A, 102T, 251T, 12V/25M/251T/256A, 12T/102T/256A, 230T/251T, 12T/230T/251S/254L/256A, 12V/38R/230T/251T/254V/256A, 12T/38R/230T, 12L/38R/254V, 12L/251T/256A, 12T/230T, 102T/230T/251S/254V/256A, 230T/251S/254V, 12T/230T/251S/254V/256A, 12T/38R/251S, 12T/230T/251S/254V, 12T/230T/251T, 156Q/248T/334R/335H, 12V/38R/230T, 12T/25M/38R, 12V/102T/230T/251T/254V, 12L/256A, 12T, 230T/251T/254L, 12V/25L/38R, 12T/38R/251S/254V, 12V/251S/254L/256A, 12T/251T/254V, 12T/102T, 12V/38R/251S, 12V/230T/251S/254L, 12T/251T/256A, 38R/230T/251S/254V/256A, 12V/230T/251T/256A, 12L/25M/230T/251S/256A, 12T/230T/311C, 12L/38R/230T/251S/254V/256A, 12L/230T/251T/254L, 12L/254V, 38R/230T/251S/254V, 12L/38R/251S/256A, 12L/38R/251S, 156Q/334R/335H, 12A/230T, 12P/230T, 12T/38R/251T, 257G/258P/334R, 12L/25L/38R/251T/256A, 334R, 12T/25L/230T/254V/256A, 12T/251S/256A, 38R/251T/256A, 12V/251S, 12V/251T/254L, 12V/38R/230T/251T/254V, 12L/38R, 12L/251S/254L/256A, 257G/258P/334R/335H, 12L/38R/254L/256A, 12V/254L, 12T/254L, 12T/38R, 232G/248T/257G/258P/334R/335H, 12L/102T, 156Q/258P/334R/335H, 156Q/249R/334R/335H, 12T/102T/230T/254L, 25M, 12V/254L/256A, 156H/334R/335H, 156Q/257G/258P/334R/335H, 12T/102T/230T, 156Q/248T/257G/258P/334R, 12T/256A, 248T/249R/334R/335H, 232G/257G/258P/334R/335H, 12T/38R/251T/254V, 334R/335H, 156Q/334R, 12V/38R/254V, 251T/254V, 12T/38R/102T/256A, 156Q/257G/334R/335H, 38R/251S/256A, 12T/140I/251S/254L, 251S/254V/256A, 12V/251T, 248T/334R/335H, 12T/254V, 12V/38R, 12V/251T/256A, 156Q/258P/334R, 156Q/248T/330R/335H, 12T/38R/256A, 232G/334R/335H, 156Q/249R/334R, 330R/334R/335H, 156Q/248T/334R, 156Q/335H, 257G/334R, 156H/248T/334R/335H, 248T/334R, 232G/248T/334R/335H, 156H/248T/334R, 156H/334R, 12T/25M/38R/230T/251S/256A, 12V/251S/254V/256A, 156Q/248T/249R/334R/335H, 330R/335H, 248T/257G/334R/335H, 232G/248T/334R, 156Q/232G/248T/334R/335H, 232G/257G/330R/334R, 156Q/248T/291T/334R/335H, 156Q/232G/248T/257G/258P/334R, 12V/38R/251T/254L, 12T/25L/38R, 156Q/232G/249R/334R/335H, 12T/25M, 156Q/248T/330R/334R/335H, 156Q/248T/249R/334R, 156H/232G/248T/334R/335H, 232G/248T/330R/335H, 156Q/232G/330R/334R/335H, 197W, 338L, 327S, 192F, 331S, 326C, 338G, 345S, 327G, 101V, 100V, 331G, 141G, 198M, 326L, 64W, 306G, 345Q, 101W, 100R, 331W, 317F, 338M, 265F, 296W, 194V, 62V, 317W, 90F, 307R, 317G, 100T, 100L, 194G, 103G, 62A, 210G, 105R, or 61A, wherein the amino acid positions are relative to the reference sequence corresponding to residues 12 to 346 of SEQ ID NO: 54, or relative to the reference sequence corresponding to SEQ ID NO: 54.

In some embodiments, the amino acid sequence of the engineered RNA ligase comprises at least a substitution or substitution set, or amino acid residue(s) T156Q/T232G/Q334R/N335H, M12V/K254V/K256A, M12V/N230T, M12L/N230T/K251T, M12L/N230T/K251S/K254V/K256A, M12A/M38R/N230T, N230T/K251S, M12P/M38R/K254V, M12V/N230T/K251T, N230T/K256A, K254V, N230T, M12T/N230T/K251S, M12T/N230T/K254V, M12V/M38R/N230T/K251T/K256A, M12L/K251S, M12T/K251T/K254V/K256A, M12V/N230T/K251S, M38R, M12T/N230T/K256A, M12L, M12V, M12L/N25M/M38R/N230T/K251T, M12L/M38R/M102T/N230T/K251T/K256A, M12L/N230T, M12V/K256A, M38R/N230T/K256A, M12V/N230T/K251S/K254V/K256A, K256A, M12T/N230T/K251S/K256A, M12T/K251T, N230T/K251S/K254L, N230T/K254V, K251S/K254L, M12T/N25L/N230T, M12V/N230T/K251S/K254L/K256A, M38R/N230T, M12T/K251S, M38R/K251T, M12V/N230T/K251S/K256A, M12L/N230T/K251T/K254V/K256A, M12T/M38R/N230T/K256A, M12L/N230T/K256A, M102T, K251T, M12V/N25M/K251T/K256A, M12T/M102T/K256A, N230T/K251T, M12T/N230T/K251S/K254L/K256A, M12V/M38R/N230T/K251T/K254V/K256A, M12T/M38R/N230T, M12L/M38R/K254V, M12L/K251T/K256A, M12T/N230T, M102T/N230T/K251S/K254V/K256A, N230T/K251S/K254V, M12T/N230T/K251S/K254V/K256A, M12T/M38R/K251S, M12T/N230T/K251S/K254V, M12T/N230T/K251T, T156Q/K248T/Q334R/N335H, M12V/M38R/N230T, M12T/N25M/M38R, M12V/M102T/N230T/K251T/K254V, M12L/K256A, M12T, N230T/K251T/K254L, M12V/N25L/M38R, M12T/M38R/K251S/K254V, M12V/K251S/K254L/K256A, M12T/K251T/K254V, M12T/M102T, M12V/M38R/K251S, M12V/N230T/K251S/K254L, M12T/K251T/K256A, M38R/N230T/K251S/K254V/K256A, M12V/N230T/K251T/K256A, M12L/N25M/N230T/K251S/K256A, M12T/N230T/R311C, M12L/M38R/N230T/K251S/K254V/K256A, M12L/N230T/K251T/K254L, M12L/K254V, M38R/N230T/K251S/K254V, M12L/M38R/K251S/K256A, M12L/M38R/K251S, T156Q/Q334R/N335H, M12A/N230T, M12P/N230T, M12T/M38R/K251T, V257G/E258P/Q334R, M12L/N25L/M38R/K251T/K256A, Q334R, M12T/N25L/N230T/K254V/K256A, M12T/K251S/K256A, M38R/K251T/K256A, M12V/K251S, M12V/K251T/K254L, M12V/M38R/N230T/K251T/K254V, M12L/M38R, M12L/K251S/K254L/K256A, V257G/E258P/Q334R/N335H, M12L/M38R/K254L/K256A, M12V/K254L, M12T/K254L, M12T/M38R, T232G/K248T/V257G/E258P/Q334R/N335H, M12L/M102T, T156Q/E258P/Q334R/N335H, T156Q/L249R/Q334R/N335H, M12T/M102T/N230T/K254L, N25M, M12V/K254L/K256A, T156H/Q334R/N335H, T156Q/V257G/E258P/Q334R/N335H, M12T/M102T/N230T, T156Q/K248T/V257G/E258P/Q334R, M12T/K256A, K248T/L249R/Q334R/N335H, T232G/V257G/E258P/Q334R/N335H, M12T/M38R/K251T/K254V, Q334R/N335H, T156Q/Q334R, M12V/M38R/K254V, K251T/K254V, M12T/M38R/M102T/K256A, T156Q/V257G/Q334R/N335H, M38R/K251S/K256A, M12T/N140I/K251S/K254L, K251S/K254V/K256A, M12V/K251T, K248T/Q334R/N335H, M12T/K254V, M12V/M38R, M12V/K251T/K256A, T156Q/E258P/Q334R, T156Q/K248T/T330R/N335H, M12T/M38R/K256A, T232G/Q334R/N335H, T156Q/L249R/Q334R, T330R/Q334R/N335H, T156Q/K248T/Q334R, T156Q/N335H, V257G/Q334R, T156H/K248T/Q334R/N335H, K248T/Q334R, T232G/K248T/Q334R/N335H, T156H/K248T/Q334R, T156H/Q334R, M12T/N25M/M38R/N230T/K251S/K256A, M12V/K251S/K254V/K256A, T156Q/K248T/L249R/Q334R/N335H, T330R/N335H, K248T/V257G/Q334R/N335H, T232G/K248T/Q334R, T156Q/T232G/K248T/Q334R/N335H, T232G/V257G/T330R/Q334R, T156Q/K248T/A291T/Q334R/N335H, T156Q/T232G/K248T/V257G/E258P/Q334R, M12V/M38R/K251T/K254L, M12T/N25L/M38R, T156Q/T232G/L249R/Q334R/N335H, M12T/N25M, T156Q/K248T/T330R/Q334R/N335H, T156Q/K248T/L249R/Q334R, T156H/T232G/K248T/Q334R/N335H, T232G/K248T/T330R/N335H, T156Q/T232G/T330R/Q334R/N335H, E197W, R338L, K327S, D192F, I331S, K326C, R338G, L345S, K327G, A101V, N100V, 1331G, E141G, H198M, K326L, V64W, I306G, L345Q, A101W, N100R, 1331W, L317F, R338M, R265F, R296W, T194V, E62V, L317W, D90F, N307R, L317G, N100T, N100L, T194G, D103G, E62A, S210G, V105R, or T61A, wherein the amino acid positions are relative to the reference sequence corresponding to residues 12 to 346 of SEQ ID NO: 54, or relative to the reference sequence corresponding to SEQ ID NO: 54.

In some embodiments, the engineered RNA ligase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12 to 346 of SEQ ID NO: 244, or to the reference sequence corresponding to SEQ ID NO: 244, wherein the amino acid sequence comprises one or more substitutions relative to the reference sequence corresponding to residues 12 to 346 of SEQ ID NO: 244, or relative to the reference sequence corresponding to SEQ ID NO: 244.

In some embodiments, the engineered RNA ligase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12 to 346 of an even-numbered SEQ ID NO. of SEQ ID NOs: 632-820, or to the reference sequence corresponding to an even-numbered SEQ ID NO. of SEQ ID NOs: 632-820, wherein the amino acid sequence comprises one or more substitutions relative to the reference sequence corresponding to residues 12 to 346 of SEQ ID NO: 244, or relative to the reference sequence corresponding to SEQ ID NO: 244.

In some embodiments, the amino acid sequence of the engineered RNA ligase comprises at least a substitution or substitution set at amino acid position(s) 141/197/326/327, 100/101/338, 141/198/326, 64/100/101, 100/338, 101/141/198/326, 101/198/326/327, 198/326, 64/100/101/338/345, 197/326, 198/327, 141/198/327, 198/326/327, 197/326/327, 64/100/338, 64/100/101/156/197/338, 64/100/101/338, 100/197/338, 197/338, 101/197/326, 64/100/197/338, 101/198, 141/197/327, 64/100/101/197/338, 101/326, 101/197/326/327, 197/327, 197, 141/197, 101, 101/198/326, 64/197/198/338, 141, 101/141, 100/101/197/338, 141/326, 338, 326, 197/198/338, 64/101/338, 64/197/198, 101/197, 100/101, 100/109/197/338, 101/197/338, 197/198, 141/326/327, 100/197, 64/100, 64/197/338, 100/197/198, 101/338, 141/327, 101/306, 64/100/101/197, 101/326/327, 100, 327, 326/345, 64/100/101/197/198, 326/327, 64/101,100/101/197, 101/327, 64/100/197/198/338, 306, 64/338,101/141/326/327, 64/101/197/338, or 101/198/306/327, wherein the amino acid positions are relative to the reference sequence corresponding to residues 12 to 346 of SEQ ID NO: 244, or relative to the reference sequence corresponding to SEQ ID NO: 244.

In some embodiments, the amino acid sequence of the engineered RNA ligase comprises at least a substitution or substitution set, or amino acid residue(s) 141G/197W/326L/327S, 100V/101V/338L, 141G/198M/326L, 64W/100V/101V, 100V/338L, 101V/141G/198M/326L, 101V/198M/326L/327G, 100V/101V/338G, 198M/326L, 64W/100V/101V/338G/345Q, 64W/100V/101V/338L/345Q, 197W/326L, 198M/327S, 141G/198M/327S, 100V/338G, 198M/326L/327G, 197W/326L/327G, 64W/100V/338G, 64W/100V/101V/156S/197W/338L, 64W/100V/101V/338G, 100V/197W/338L, 198M/326L/327S, 197W/338L, 101V/197W/326L, 64W/100V/197W/338L, 101V/198M, 141G/197W/327S, 198M/327G, 198M/326C/327S, 64W/100V/101V/197W/338G, 101V/326L, 101V/197W/326L/327G, 141G/197W/326L/327G, 100V/101W/338G, 197W/327S, 197W, 141G/197W, 101V, 101V/198M/326C, 64W/197W/198M/338G, 141G, 64W/100V/101W/338G, 101V/141G, 100V/101V/197W/338L, 141G/326C, 338L, 197W/338G, 326L, 197W/198M/338G, 197W/198M/338L, 64W/101W/338L, 64W/197W/198M, 101V/197W, 100V/101V, 141G/326L, 100V/109F/197W/338L, 101W/197W/338L, 64W/100V/101W, 197W/198M, 100V/101W/338L, 141G/326C/327S, 100V/197W, 197W/326C/327G, 64W/100V, 101V/326C, 100V/101W, 64W/197W/338L, 100V/197W/198M, 101W, 101W/338L, 141G/327S, 101V/306G, 64W/100V/101W/197W, 101V/326L/327G, 100V, 327G, 326C/345S, 64W/100V/101V/197W/198M, 326C/327S, 64W/101W, 100V/101V/197W, 101V/327S, 338G, 100V/101V/197W/338G, 100V/101W/197W/338G, 64W/100V/197W/198M/338G, 306G, 64W/338G, 64W/100V/101V/197W, 64W/197W/198M/338L, 101V/327G, 101V/141G/326C/327S, 64W/101W/197W/338L, 101W/338G, or 101V/198M/306G/327S, wherein the amino acid positions are relative to the reference sequence corresponding to residues 12 to 346 of SEQ ID NO: 244, or relative to the reference sequence corresponding to SEQ ID NO: 244.

In some embodiments, the amino acid sequence of the engineered RNA ligase comprises at least a substitution or substitution set, or amino acid residue(s) E141G/E197W/K326L/K327S, N100V/A101V/R338L, E141G/H198M/K326L, V64W/N100V/A101V, N100V/R338L, A101V/E141G/H198M/K326L, A101V/H198M/K326L/K327G, N100V/A101V/R338G, H198M/K326L, V64W/N100V/A101V/R338G/L345Q, V64W/N100V/A101V/R338L/L345Q, E197W/K326L, H198M/K327S, E141G/H198M/K327S, N100V/R338G, H198M/K326L/K327G, E197W/K326L/K327G, V64W/N100V/R338G, V64W/N100V/A101V/T156S/E197W/R338L, V64W/N100V/A101V/R338G, N100V/E197W/R338L, H198M/K326L/K327S, E197W/R338L, A101V/E197W/K326L, V64W/N100V/E197W/R338L, A101V/H198M, E141G/E197W/K327S, H198M/K327G, H198M/K326C/K327S, V64W/N100V/A101V/E197W/R338G, A101V/K326L, A101V/E197W/K326L/K327G, E141G/E197W/K326L/K327G, N100V/A101W/R338G, E197W/K327S, E197W, E141G/E197W, A101V, A101V/H198M/K326C, V64W/E197W/H198M/R338G, E141G, V64W/N100V/A101W/R338G, A101V/E141G, N100V/A101V/E197W/R338L, E141G/K326C, R338L, E197W/R338G, K326L, E197W/H198M/R338G, E197W/H198M/R338L, V64W/A101W/R338L, V64W/E197W/H198M, A101V/E197W, N100V/A101V, E141G/K326L, N100V/I109F/E197W/R338L, A101W/E197W/R338L, V64W/N100V/A101W, E197W/H198M, N100V/A101W/R338L, E141G/K326C/K327S, N100V/E197W, E197W/K326C/K327G, V64W/N100V, A101V/K326C, N100V/A101W, V64W/E197W/R338L, N100V/E197W/H198M, A101W, A101W/R338L, E141G/K327S, A101V/I306G, V64W/N100V/A101W/E197W, A101V/K326L/K327G, N100V, K327G, K326C/L345S, V64W/N100V/A101V/E197W/H198M, K326C/K327S, V64W/A101W, N100V/A101V/E197W, A101V/K327S, R338G, N100V/A101V/E197W/R338G, N100V/A101W/E197W/R338G, V64W/N100V/E197W/H198M/R338G, I306G, V64W/R338G, V64W/N100V/A101V/E197W, V64W/E197W/H198M/R338L, A101V/K327G, A101V/E141G/K326C/K327S, V64W/A101W/E197W/R338L, A101W/R338G, or A101V/H198M/1306G/K327S, wherein the amino acid positions are relative to the reference sequence corresponding to residues 12 to 346 of SEQ ID NO: 244, or relative to the reference sequence corresponding to SEQ ID NO: 244.

In some embodiments, the engineered RNA ligase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12 to 346 of SEQ ID NO: 648, or to the reference sequence corresponding to SEQ ID NO: 648, wherein the amino acid sequence comprises one or more substitutions relative to the reference sequence corresponding to residues 12 to 346 of SEQ ID NO: 648, or relative to the reference sequence corresponding to SEQ ID NO: 648.

In some embodiments, the engineered RNA ligase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12 to 346 of an even-numbered SEQ ID NO. of SEQ ID NOs: 822-954, or to the reference sequence corresponding to an even-numbered SEQ ID NO. of SEQ ID NOs: 822-954, wherein the amino acid sequence comprises one or more substitutions relative to the reference sequence corresponding to residues 12 to 346 of SEQ ID NO: 648, or relative to the reference sequence corresponding to SEQ ID NO: 648.

In some embodiments, the amino acid sequence of the engineered RNA ligase comprises at least a substitution or substitution set at amino acid position(s) 194/330/331, 298, 331, 192/193/194/298, 330, 192/330/331, 192/193/194/331, 298/330/331, 194/298/330/331, 192/193/330/331, 192/298/331, 192/193, 194/330, 60/100/156, 193/296/331, 298/331, 193/194/330/331, 194, 194/298/331, 193/298, 193/330, 192, 192/193/194/298/331, 192/193/194/298/330, 192/193/298/331, 100/156, 62/100,298/330, 60/100, 192/193/298/330/331, 100, 193/194/298, 338, 62, 247, 18, 37, 254, 33, 261, 251, 260, 12, 117, 256, 255, 38, 29, 25, or 252, wherein the amino acid positions are relative to the reference sequence corresponding to residues 12 to 346 of SEQ ID NO: 648, or relative to the reference sequence corresponding to SEQ ID NO: 648.

In some embodiments, the amino acid sequence of the engineered RNA ligase comprises at least a substitution or substitution set, or amino acid residue(s) 194V/330D/331W, 298S, 331W, 192L/193K/194V/298S, 330D, 192L/330D/331G, 192L/193K/194V/331S, 2981/330D/331S, 194V/298S/330D/331G, 192L/193K/330D/331S, 192L/298S/331S, 192L/193K, 194V/330D, 331G, 192L/298S/331W, 60D/100R/156G, 193K/296G/331W, 298S/331G, 193K/194V/330D/331W, 194V, 194V/298S/331G, 193K/298S, 193K/330D, 192L, 192L/193K/194V/298S/331S, 192L/193K/194V/298S/330D, 192L/193K/298S/331S, 100R/156G, 298S/331S, 2981/331G, 62V/100V, 298S/330D, 193K/2981, 331S, 60D/100R, 192L/193K/298S/330D/331S, 192L/193K/194V/298S/331W, 192L/330D/331S, 100R, 193K/194V/298S, 194V/298S/331S, 338L, 62V, 247S, 18H, 37A, 254S, 33L, 261T, 251Y, 260G, 251C, 12R, 117A, 256S, 255Y, 255S, 38E, 12M, 251T, 255V, 29D, 247R, 25A, 256E, 252A, or 261D, wherein the amino acid positions are relative to the reference sequence corresponding to residues 12 to 346 of SEQ ID NO: 648, or relative to the reference sequence corresponding to SEQ ID NO: 648.

In some embodiments, the amino acid sequence of the engineered RNA ligase comprises at least a substitution or substitution set, or amino acid residue(s) T194V/T330D/1331W, M298S, 1331W, D192L/T193K/T194V/M298S, T330D, D192L/T330D/1331G, D192L/T193K/T194V/1331S, M298I/T330D/1331S, T194V/M298S/T330D/1331G, D192L/T193K/T330D/1331S, D192L/M298S/1331S, D192L/T193K, T194V/T330D, I331G, D192L/M298S/1331W, P60D/N100R/T156G, T193K/R296G/1331W, M298S/1331G, T193K/T194V/T330D/1331W, T194V, T194V/M298S/1331G, T193K/M298S, T193K/T330D, D192L, D192L/T193K/T194V/M298S/1331S, D192L/T193K/T194V/M298S/T330D, D192L/T193K/M298S/1331S, N100R/T156G, M298S/1331S, M298I/1331G, E62V/N100V, M298S/T330D, T193K/M298I, I331S, P60D/N100R, D192L/T193K/M298S/T330D/1331S, D192L/T193K/T194V/M298S/1331W, D192L/T330D/1331S, N100R, T193K/T194V/M298S, T194V/M298S/1331S, R338L, E62V, A247S, S18H, L37A, K254S, G33L, E261T, S251Y, T260G, S251C, T12R, G117A, K256S, A255Y, A255S, M38E, T12M, S251T, A255V, E29D, A247R, N25A, K256E, P252A, or E261D, wherein the amino acid positions are relative to the reference sequence corresponding to residues 12 to 346 of SEQ ID NO: 648, or relative to the reference sequence corresponding to SEQ ID NO: 648.

In some embodiments, the engineered RNA ligase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12 to 346 of SEQ ID NO: 862, or to the reference sequence corresponding to SEQ ID NO: 862, wherein the amino acid sequence comprises one or more substitutions relative to the reference sequence corresponding to residues 12 to 346 of SEQ ID NO: 862, or relative to the reference sequence corresponding to SEQ ID NO: 862.

In some embodiments, the engineered RNA ligase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12 to 346 of an even-numbered SEQ ID NO. of SEQ ID NOs: 956-1078, or to the reference sequence corresponding to an even-numbered SEQ ID NO. of SEQ ID NOs: 956-1078, wherein the amino acid sequence comprises one or more substitutions relative to the reference sequence corresponding to residues 12 to 346 of SEQ ID NO: 862, or relative to the reference sequence corresponding to SEQ ID NO: 862.

In some embodiments, the amino acid sequence of the engineered RNA ligase comprises at least a substitution or substitution set at amino acid positions(s) 254, 18/254,247/317, 37/317/342, 33, 18/156/254, 18, 156, 254/338, 254/307, 33/37/317, 37, 37/156/317, 156/265/317, 265/317, 156/254, 33/156/317, 317, 156/317, 37/317, 18/338,338, 156/247/317, 33/317, 37/247/265/317, 33/37/156/317, 156/247/296/317, 18/307,156/296/317, 156/247, 37/247/317, 18/254/307, 307, 265, 33/37, 18/156, 33/156/296/317, 18/156/254/338, 37/265/317, 33/37/156/247/317, 296, 33/296/317, 156/296, 247/296/317, 37/296/317, 247/296, 33/37/156, 296/317, 156/254/307, 18/156/307, or 33/37/265, wherein the amino acid positions are relative to the reference sequence corresponding to residues 12 to 346 of SEQ ID NO: 862, or relative to the reference sequence corresponding to SEQ ID NO: 862.

In some embodiments, the amino acid sequence of the engineered RNA ligase comprises at least a substitution or substitution set, or amino acid residue(s) 254S, 18H/254S, 247S/317W, 37A/317W/342R, 33L, 247S/317F, 18H/156G/254S, 18H, 156G, 254S/338L, 254S/307R, 33L/37A/317W, 37A, 37A/156G/317W, 156G/265F/317W, 265F/317W, 156G/254S, 33L/156G/317W, 317F, 156G/317F, 317W, 156G/317W, 37A/317W, 18H/338M, 338M, 156G/247S/317W, 18H/338L, 33L/317W, 37A/247S/265F/317F, 37A/317F, 33L/37A/156G/317W, 156G/247S/296W/317W, 18H/307R, 156G/296W/317W, 156G/247S, 37A/247S/317F, 156G/265F/317F, 18H/254S/307R, 307R, 265F, 265F/317F, 33L/37A, 18H/156G, 33L/156G/296W/317W, 18H/156G/254S/338L, 37A/265F/317F, 33L/37A/156G/247S/317W, 296W, 33L/296W/317W, 156G/247S/317F, 156G/296W, 338L, 37A/265F/317W, 247S/296W/317F, 33L/156G/317F, 37A/296W/317F, 247S/296W, 33L/37A/156G, 296W/317W, 156G/254S/307R, 18H/156G/307R, or 33L/37A/265F, wherein the amino acid positions are relative to the reference sequence corresponding to residues 12 to 346 of SEQ ID NO: 862, or relative to the reference sequence corresponding to SEQ ID NO: 862.

In some embodiments, the amino acid sequence of the engineered RNA ligase comprises at least a substitution or substitution set, or amino acid residue(s) K254S, S18H/K254S, A247S/L317W, L37A/L317W/H342R, G33L, A247S/L317F, S18H/T156G/K254S, S18H, T156G, K254S/R338L, K254S/N307R, G33L/L37A/L317W, L37A, L37A/T156G/L317W, T156G/R265F/L317W, R265F/L317W, T156G/K254S, G33L/T156G/L317W, L317F, T156G/L317F, L317W, T156G/L317W, L37A/L317W, S18H/R338M, R338M, T156G/A247S/L317W, S18H/R338L, G33L/L317W, L37A/A247S/R265F/L317F, L37A/L317F, G33L/L37A/T156G/L317W, T156G/A247S/R296W/L317W, S18H/N307R, T156G/R296W/L317W, T156G/A247S, L37A/A247S/L317F, T156G/R265F/L317F, S18H/K254S/N307R, N307R, R265F, R265F/L317F, G33L/L37A, S18H/T156G, G33L/T156G/R296W/L317W, S18H/T156G/K254S/R338L, L37A/R265F/L317F, G33L/L37A/T156G/A247S/L317W, R296W, G33L/R296W/L317W, T156G/A247S/L317F, T156G/R296W, R338L, L37A/R265F/L317W, A247S/R296W/L317F, G33L/T156G/L317F, L37A/R296W/L317F, A247S/R296W, G33L/L37A/T156G, R296W/L317W, T156G/K254S/N307R, S18H/T156G/N307R, or G33L/L37A/R265F, wherein the amino acid positions are relative to the reference sequence corresponding to residues 12 to 346 of SEQ ID NO: 862, or relative to the reference sequence corresponding to SEQ ID NO: 862.

In some embodiments, the engineered RNA ligase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12 to 346 of SEQ ID NO: 956, or to the reference sequence corresponding to SEQ ID NO: 956, wherein the amino acid sequence comprises one or more substitutions relative to the reference sequence corresponding to residues 12 to 346 of SEQ ID NO: 956, or relative to the reference sequence corresponding to SEQ ID NO: 956.

In some embodiments, the engineered RNA ligase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12 to 346 of an even-numbered SEQ ID NO. of SEQ ID NOs: 1080-1374, or to the reference sequence corresponding to an even-numbered SEQ ID NO. of SEQ ID NOs: 1080-1374, wherein the amino acid sequence comprises one or more substitutions relative to the reference sequence corresponding to residues 12 to 346 of SEQ ID NO: 956, or relative to the reference sequence corresponding to SEQ ID NO: 956.

In some embodiments, the amino acid sequence of the engineered RNA ligase comprises at least a substitution or substitution set at amino acid position(s) 12/25/29/156, 12/255/256/257, 12/156/255, 247/252, 12/25, 12, 12/25/29/156/255, 256, 12/156, 12/25/156/255/256/257/261, 18/247/251, 25/255/256/257, 12/25/257, 255/257, 12/25/29/255/257, 256/257, 12/255/257, 12/156/255/256/257, 25, 18/38/251/252, 12/256/257/261, 12/29, 12/25/255/256/257/261, 38, 12/25/29/256/257, 29/257, 12/261,251, 249/251, 12/255, 12/29/156/256/257, 252, 255/257/261, 18, 29/255/257, 255/256/257, 12/25/29/156/256/257, 25/255/256/257/261, 29/255/256/257/261, 25/256, 18/38/247/252/327, 18/247, 25/29, 12/25/156/255/257, 18/156/251/252, 12/255/261, 25/255, 12/255/256/261, 156/255/257, 156/257, 12/156/256/257, 25/156/256/257, 12/25/29/156/257/261, 25/261, 12/156/255/256/261, 12/25/29/255/256/257, 38/252,255, 249/252, 12/156/255/256, 12/25/29/261, 257, 12/255/256, 12/29/255, 12/255/257/261, 12/156/257, 192/249/251/252, 12/25/29/255/261, 12/156/255/257, 12/29/255/257, 12/29/256/257, 18/252,249, 156/250, 38/251/252, 156/255/256/257, 25/257, 12/255/256/257/261, 25/156/261, 156/252, 12/25/255/256/257, 12/25/29/156/255/257, 29/156,251/252, 256/257/261, 25/156/255/256/257, 156/249/251/252, 25/29/255, 12/29/156, 255/256, 38/250/252, 247/249/252, 12/256/257, 60, 12/256, 12/156/256, 25/255/257/261, 12/25/29/255/256/261, 12/29/156/255/256, 12/25/156/255/256, 247/251/252, 256/261, 38/251, 12/25/29/156/261, 12/257,156/255/256/261, 12/25/156/255, 12/25/29/156/255/257/261, 12/29/256/261, 29/156/261, 25/29/156/257/261, 12/25/255/256/261, or 12/156/255/261, wherein the amino acid positions are relative to the reference sequence corresponding to residues 12 to 346 of SEQ ID NO: 956, or relative to the reference sequence corresponding to SEQ ID NO: 956.

In some embodiments, the amino acid sequence of the engineered RNA ligase comprises at least a substitution or substitution set, or amino acid residue(s) 12M/25A/29D/156G, 12M/255Y/256S/257D, 12M/156G/255V, 247R/252A, 12M/25A, 12M, 12M/25A/29D/156G/255S, 256S, 12M/156G, 12M/25A/156G/255V/256S/257D/261D, 18H/247R/251T, 25A/255V/256S/257D, 12M/25A/257D, 255S/257D, 12M/25A/29D/255Y/257D, 256S/257D, 12M/255S/257D, 12M/156G/255N/256S/257D, 25A, 18H/38E/251T/252A, 12M/256S/257D/261D, 12M/29D, 12M/25A/255V/256S/257D/261D, 38E, 12M/25A/29D/256S/257D, 29D/257D, 12M/261D, 251T, 12M/156G/255N, 249R/251T, 12M/255V, 12M/29D/156G/256S/257D, 252A, 255V/257D/261D, 18A, 29D/255N/257D, 255Y/256S/257D, 12M/25A/29D/156G/256S/257D, 25A/255V/256S/257D/261D, 29D/255Y/256S/257D/261D, 25A/256S, 18H/38E/247R/252A/327M, 18H/247R, 25A/29D, 12M/25A/156G/255V/257D, 255V/257D, 12M/255N, 18H/156G/251T/252A, 12M/255V/261D, 25A/255V, 12M/255V/256S/261D, 156G/255S/257D, 156G/257D, 12M/156G/256S/257D, 12M/156G/255Y, 25A/156G/256S/257D, 12M/25A/29D/156G/257D/261D, 25A/261D, 12M/156G/255Y/256S/261D, 12M/25A/29D/255Y/256S/257D, 38E/252A, 12M/156G/255V/256S/257D, 255S, 249R/252A, 12M/156G/255Y/256S, 12M/25A/29D/261D, 257D, 12M/255N/256S, 12M/29D/255N, 12M/255V/257D/261D, 12M/255Y/257D, 12M/156G/257D, 192N/249R/251T/252A, 12M/25A/29D/255N/256S/257D, 12M/25A/29D/255V/261D, 12M/156G/255Y/257D, 12M/29D/255V/257D, 12M/29D/256S/257D, 18A/252A, 249R, 156G/250I, 38E/251T/252A, 156G/255Y/256S/257D, 25A/257D, 12M/255V/256S/257D/261D, 25A/156G/261D, 156G/252A, 12M/25A/255V/256S/257D, 12M/25A/29D/156G/255S/257D, 29D/156G, 251T/252A, 256S/257D/261D, 25A/156G/255Y/256S/257D, 12M/156G/255Y/256S/257D, 156G/249R/251T/252A, 25A/29D/255V, 12M/29D/156G, 255V/256S, 12M/255Y/256S, 38E/250I/252A, 247R/249R/252A, 12M/256S/257D, 12M/156G/255S/257D, 12M/156G/255V/257D, 60S, 12M/255Y/256S/261D, 12M/256S, 12M/255S, 255S/257D/261D, 12M/156G/256S, 12M/255Y/257D/261D, 25A/255V/257D/261D, 12M/25A/29D/255N/256S/261D, 12M/255V/257D, 12M/29D/156G/255V/256S, 255S/256S, 12M/25A/156G/255V/256S, 247R/251T/252A, 256S/261D, 38E/251T, 12M/25A/29D/156G/261D, 12M/257D, 156G/255N/256S/261D, 12M/25A/156G/255S, 25A/255S, 12M/25A/29D/156G/255V/257D/261D, 12M/29D/256S/261D, 156G/255V/257D, 29D/156G/261D, 12M/255S/257D/261D, 25A/29D/156G/257D/261D, 12M/25A/255V/256S/261D, or 12M/156G/255V/261D, wherein the amino acid positions are relative to the reference sequence corresponding to residues 12 to 346 of SEQ ID NO: 956, or relative to the reference sequence corresponding to SEQ ID NO: 956.

In some embodiments, the amino acid sequence of the engineered RNA ligase comprises at least a substitution or substitution set, or amino acid residue(s) T12M/N25A/E29D/T156G, T12M/A255Y/K256S/V257D, T12M/T156G/A255V, A247R/P252A, T12M/N25A, T12M, T12M/N25A/E29D/T156G/A255S, K256S, T12M/T156G, T12M/N25A/T156G/A255V/K256S/V257D/E261D, S18H/A247R/S251T, N25A/A255V/K256S/V257D, T12M/N25A/V257D, A255S/V257D, T12M/N25A/E29D/A255Y/V257D, K256S/V257D, T12M/A255S/V257D, T12M/T156G/A255N/K256S/V257D, N25A, S18H/M38E/S251T/P252A, T12M/K256S/V257D/E261D, T12M/E29D, T12M/N25A/A255V/K256S/V257D/E261D, M38E, T12M/N25A/E29D/K256S/V257D, E29D/V257D, T12M/E261D, S251T, T12M/T156G/A255N, L249R/S251T, T12M/A255V, T12M/E29D/T156G/K256S/V257D, P252A, A255V/V257D/E261D, S18A, E29D/A255N/V257D, A255Y/K256S/V257D, T12M/N25A/E29D/T156G/K256S/V257D, N25A/A255V/K256S/V257D/E261D, E29D/A255Y/K256S/V257D/E261D, N25A/K256S, S18H/M38E/A247R/P252A/K327M, S18H/A247R, N25A/E29D, T12M/N25A/T156G/A255V/V257D, A255V/V257D, T12M/A255N, S18H/T156G/S251T/P252A, T12M/A255V/E261D, N25A/A255V, T12M/A255V/K256S/E261D, T156G/A255S/V257D, T156G/V257D, T12M/T156G/K256S/V257D, T12M/T156G/A255Y, N25A/T156G/K256S/V257D, T12M/N25A/E29D/T156G/V257D/E261D, N25A/E261D, T12M/T156G/A255Y/K256S/E261D, T12M/N25A/E29D/A255Y/K256S/V257D, M38E/P252A, T12M/T156G/A255V/K256S/V257D, A255S, L249R/P252A, T12M/T156G/A255Y/K256S, T12M/N25A/E29D/E261D, V257D, T12M/A255N/K256S, T12M/E29D/A255N, T12M/A255V/V257D/E261D, T12M/A255Y/V257D, T12M/T156G/V257D, D192N/L249R/S251T/P252A, T12M/N25A/E29D/A255N/K256S/V257D, T12M/N25A/E29D/A255V/E261D, T12M/T156G/A255Y/V257D, T12M/E29D/A255V/V257D, T12M/E29D/K256S/V257D, S18A/P252A, L249R, T156G/D250I, M38E/S251T/P252A, T156G/A255Y/K256S/V257D, N25A/V257D, T12M/A255V/K256S/V257D/E261D, N25A/T156G/E261D, T156G/P252A, T12M/N25A/A255V/K256S/V257D, T12M/N25A/E29D/T156G/A255S/V257D, E29D/T156G, S251T/P252A, K256S/V257D/E261D, N25A/T156G/A255Y/K256S/V257D, T12M/T156G/A255Y/K256S/V257D, T156G/L249R/S251T/P252A, N25A/E29D/A255V, T12M/E29D/T156G, A255V/K256S, T12M/A255Y/K256S, M38E/D250I/P252A, A247R/L249R/P252A, T12M/K256S/V257D, T12M/T156G/A255S/V257D, T12M/T156G/A255V/V257D, P60S, T12M/A255Y/K256S/E261D, T12M/K256S, T12M/A255S, A255S/V257D/E261D, T12M/T156G/K256S, T12M/A255Y/V257D/E261D, N25A/A255V/V257D/E261D, T12M/N25A/E29D/A255N/K256S/E261D, T12M/A255V/V257D, T12M/E29D/T156G/A255V/K256S, A255S/K256S, T12M/N25A/T156G/A255V/K256S, A247R/S251T/P252A, K256S/E261D, M38E/S251T, T12M/N25A/E29D/T156G/E261D, T12M/V257D, T156G/A255N/K256S/E261D, T12M/N25A/T156G/A255S, N25A/A255S, T12M/N25A/E29D/T156G/A255V/V257D/E261D, T12M/E29D/K256S/E261D, T156G/A255V/V257D, E29D/T156G/E261D, T12M/A255S/V257D/E261D, N25A/E29D/T156G/V257D/E261D, T12M/N25A/A255V/K256S/E261D, or T12M/T156G/A255V/E261D, wherein the amino acid positions are relative to the reference sequence corresponding to residues 12 to 346 of SEQ ID NO: 956, or relative to the reference sequence corresponding to SEQ ID NO: 956.

In some embodiments, the amino acid sequence of the engineered RNA ligase comprises at least a substitution at amino acid position 157, 183, 95, 185, 181, 213, 89, 28, 190, 214, or 143, or any combinations thereof, wherein the amino acid positions are relative to the reference sequence corresponding to residues 12 to 346 of SEQ ID NO: 956, or relative to the reference sequence corresponding to SEQ ID NO: 956.

In some embodiments, the amino acid sequence of the engineered RNA ligase comprises at least a substitution, or amino acid residue 157V, 183L, 95A, 183V, 185P, 181E, 213D, 89M, 89G, 28M, 190M, 214L, 143A, 89Q, or 95G, or any combinations thereof, wherein the amino acid positions are relative to the reference sequence corresponding to residues 12 to 346 of SEQ ID NO: 956, or relative to the reference sequence corresponding to SEQ ID NO: 956.

In some embodiments, the amino acid sequence of the engineered RNA ligase comprises at least a substitution, or amino acid residue L157V, I183L, V95A, I183V, N185P, K181E, G213D, K89M, K89G, I28M, L190M, V214L, V143A, K89Q, or V95G, wherein the amino acid positions are relative to the reference sequence corresponding to residues 12 to 346 of SEQ ID NO: 956, or relative to the reference sequence corresponding to SEQ ID NO: 956.

In some embodiments, the engineered RNA ligase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12 to 346 of SEQ ID NO: 1088, or to the reference sequence corresponding to SEQ ID NO: 1088, wherein the amino acid sequence comprises one or more substitutions relative to the reference sequence corresponding to residues 12 to 346 of SEQ ID NO: 1088, or relative to the reference sequence corresponding to SEQ ID NO: 1088.

In some embodiments, the engineered RNA ligase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12 to 346 of an even-numbered SEQ ID NO. of SEQ ID NOs: 1376-1434, or to the reference sequence corresponding to an even-numbered SEQ ID NO. of SEQ ID NOs: 1376-1434, wherein the amino acid sequence comprises one or more substitutions relative to the reference sequence corresponding to residues 12 to 346 of SEQ ID NO: 1088, or relative to the reference sequence corresponding to SEQ ID NO: 1088.

In some embodiments, the amino acid sequence of the engineered RNA ligase comprises at least a substitution or substitution set at amino acid position(s) 95/157, 95/183/185/214, 89/183, 28/89/143/183, 28/143/157, 181/183, 143/181/183/214, 143/181/183, 89/143/183, 28/143/181/183, 143/157, 95/181/183, 157, 143/183, 89/143/157/183, 89/95/157, 89/157, 89/95/143/181, 95/143, 89/157/183/214, 143/214, 143/181/183/185, 28/95/143, 143/183/214, 28/95/183/214, 89/143/157, 28/95, 95/143/183/185, or 95/143/157/183, wherein the amino acid positions are relative to the reference sequence corresponding to residues 12 to 346 of SEQ ID NO: 1088, or relative to the reference sequence corresponding to SEQ ID NO: 1088.

In some embodiments, the amino acid sequence of the engineered RNA ligase comprises at least a substitution or substitution set, or amino acid residue(s) 95A/157V, 95G/183V/185P/214L, 89M/183V, 28M/89G/143M/183L, 28M/143M/157V, 181E/183V, 143A/181E/183V/214L, 143A/181E/183V, 89G/143M/183L, 28M/143M/181E/183L, 143M/157V, 95A/181E/183L, 157V, 143A/183L, 143A/157V, 89G/143A/157V/183L, 89M/95A/157V, 89M/157V, 89G/95A/143A/181E, 95A/143A, 89M/157V/183L/214L, 143M/214L, 143M/181E/183V/185P, 28M/95A/143A, 143M/183L/214L, 28M/95A/183V/214L, 89M/143A/157V, 28M/95A, 95A/143M/183V/185P, or 95A/143A/157V/183L, wherein the amino acid positions are relative to the reference sequence corresponding to residues 12 to 346 of SEQ ID NO: 1088, or relative to the reference sequence corresponding to SEQ ID NO: 1088.

In some embodiments, the amino acid sequence of the engineered RNA ligase comprises at least a substitution or substitution set, or amino acid residue(s) V95A/L157V, V95G/1183V/N185P/V214L, K89M/I183V, I28M/K89G/V143M/I183L, I28M/V143M/L157V, K181E/I183V, V143A/K181E/I183V/V214L, V143A/K181E/I183V, K89G/V143M/1183L, I28M/V143M/K181E/I183L, V143M/L157V, V95A/K181E/I183L, L157V, V143A/I183L, V143A/L157V, K89G/V143A/L157V/1183L, K89M/V95A/L157V, K89M/L157V, K89G/V95A/V143A/K181E, V95A/V143A, K89M/L157V/I183L/V214L, V143M/V214L, V143M/K181E/I183V/N185P, I28M/V95A/V143A, V143M/I183L/V214L, I28M/V95A/I183V/V214L, K89M/V143A/L157V, I28M/V95A, V95A/V143M/I183V/N185P, or V95A/V143A/L157V/1183L, wherein the amino acid positions are relative to the reference sequence corresponding to residues 12 to 346 of SEQ ID NO: 1088, or relative to the reference sequence corresponding to SEQ ID NO: 1088.

In some embodiments, the engineered RNA ligase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12 to 346 of SEQ ID NO: 1416, or to the reference sequence corresponding to SEQ ID NO: 1416, wherein the amino acid sequence comprises one or more substitutions relative to the reference sequence corresponding to residues 12 to 346 of SEQ ID NO: 1416, or relative to the reference sequence corresponding to SEQ ID NO: 1416.

In some embodiments, the engineered RNA ligase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12 to 346 of an even-numbered SEQ ID NO. of SEQ ID NOs: 1436-1470, or to the reference sequence corresponding to an even-numbered SEQ ID NO. of SEQ ID NOs: 1436-1470, wherein the amino acid sequence comprises one or more substitutions relative to the reference sequence corresponding to residues 12 to 346 of SEQ ID NO: 1416, or relative to the reference sequence corresponding to SEQ ID NO: 1416.

In some embodiments, the amino acid sequence of the engineered RNA ligase comprises at least a substitution or substitution set at amino acid position(s) 83/137/150, 137/213, 137, 150/190, 143/150, 104/150, 60/143/150/190, 60/150/190/213, 104/137/154, 123/137, 137/150/154/190, 123/143/150, 60/104/143/150/213, 60/95/143/190, 123/137/190, 60/123,104/123, to 60/123/143, wherein the amino acid positions are relative to the reference sequence corresponding to residues 12 to 346 of SEQ ID NO: 1416, or relative to the reference sequence corresponding to SEQ ID NO: 1416.

In some embodiments, the amino acid sequence of the engineered RNA ligase comprises at least a substitution or substitution set, or amino acid residue(s) 83L/137R/150D, 137R/213R, 137R, 150D/190M, 143A/150D, 104T/150D, 60A/143A/150D/190M, 60A/150D/190M/213R, 104T/137R/154V, 123K/137R, 137R/150D/154V/190M, 123K/143T/150D, 60A/104T/143A/150D/213R, 60A/95G/143T/190M, 123R/137R/190M, 60A/123R, 104T/123R, or 60A/123R/143A, wherein the amino acid positions are relative to the reference sequence corresponding to residues 12 to 346 of SEQ ID NO: 1416, or relative to the reference sequence corresponding to SEQ ID NO: 1416.

In some embodiments, the amino acid sequence of the engineered RNA ligase comprises at least a substitution or substitution set, or amino acid residue(s) I83L/D137R/V150D, D137R/G213R, D137R, V150D/L190M, V143A/V150D, L104T/V150D, P60A/V143A/V150D/L190M, P60A/V150D/L190M/G213R, L104T/D137R/C154V, D123K/D137R, D137R/V150D/C154V/L190M, D123K/V143T/V150D, P60A/L104T/V143A/V150D/G213R, P60A/V95G/V143T/L190M, D123R/D137R/L190M, P60A/D123R, L104T/D123R, or P60A/D123R/V143A, wherein the amino acid positions are relative to the reference sequence corresponding to residues 12 to 346 of SEQ ID NO: 1416, or relative to the reference sequence corresponding to SEQ ID NO: 1416.

In some embodiments, the amino acid sequence of the engineered RNA ligase comprises at least a substitution or substitution set at amino acid position(s) 38, 249, 255, 23, 33, 25, 226, 29, 37, 10, 250, 75, 230, 77, 251, 228, 81, 224, 64, 254, 268, 65, 232, 332, 331, 330, 101, 307, 137, 223, 280, 289, 288, 342, 93, 296, 86, 241, 126, 277, 87, 247, 243, 84, 83, 192, 105, 25/29, 25/29/250, 25/29/75/77/250/255, 25/75/230, 25/77/226/255, 25/230,250/255, 29/75/226/230/255, 29/75/77, 29/75/77/226, 29/75/77/230/255, 29/226, 29/226/230, 29/230/255, 33/228, 33/249, 33/37/228, 33/37/38/228, 33/37/38/228/249, 33/38, 75/250/255, 75/226, 75/226/250/255, 75/226/230/250/255, 75/77, 75/77/255, 75/77/250/255, 75/77/226/255, 75/77/226/250/255, 75/77/230, 75/77/230/250, 75/230, 75/230/255, 75/230/250, 226/255, 226/250, 226/230, 226/230/255, 37/228, 37/228/249, 37/38, 37/38/228, 37/38/228/249, 37/38/249, 38/249/291, 10/228, 10/228/249, 10/33/249, 10/33/37, 10/33/37/38/249, 10/33/38/249, 10/33/38, 10/33/249/251, 10/33/37/228, 10/33/37/249, 10/33/37/38/228/249, 10/33/37/38, 10/249, 10/37, 10/37/228, 10/37/228/249, 10/37/249, 10/37/38/228, 10/37/38/228/249, 10/37/38, 10/38/249, 10/38, 10/38/228, 77/255, 77/250, 77/250/255, 77/226, 77/226/255, 77/230, 77/230/255, 230/255, 230/250, 230/250/255, 23/25/226/255, 23/25/226/230, or 23/25/230, wherein the amino acid positions are relative to the reference sequence corresponding to residues 12 to 346 of SEQ ID NO: 1416, or relative to the reference sequence corresponding to SEQ ID NO: 1416.

In some embodiments, the amino acid sequence of the engineered RNA ligase comprises at least a substitution or substitution set, or amino acid residue(s) 38V, 249K, 255S, 23K, 33L, 25G, 226R, 249S, 29S, 37M, 38A, 10F, 250S, 75D, 230V, 77M, 251E, 23T, 33V, 228L, 81Y, 224A, 64Q, 254K, 268F, 65C, 232N, 332E, 37K, 331R, 330K, 101P, 268G, 307R, 224Q, 137N, 223C, 280N, 2891, 288E, 342T, 93K, 268M, 296Q, 86G, 241Q, 126M, 277A, 87E, 2471, 243H, 84V, 83A, 192E, 105K, 25G/29S, 25G/29S/250S, 25G/29S/75D/77M/250S/255S, 25G/75D/230V, 25G/77M/226R/255S, 25G/230V, 250S/255S, 29S/75D/226R/230V/255S, 29S/75D/77M, 29S/75D/77M/226R, 29S/75D/77M/230V/255S, 29S/226R, 29S/226R/230V, 29S/230V/255S, 33V/228L, 33V/249K, 33V/249S, 33V/37M/228L, 33V/37M/38A/228L, 33V/37M/38A/228L/249K, 33V/38V, 75D/250S/255S, 75D/226R, 75D/226R/250S/255S, 75D/226R/230V/250S/255S, 75D/77M, 75D/77M/255S, 75D/77M/250S/255S, 75D/77M/226R/255S, 75D/77M/226R/250S/255S, 75D/77M/230V, 75D/77M/230V/250S, 75D/230V, 75D/230V/255S, 75D/230V/250S, 226R/255S, 226R/250S, 226R/230V, 226R/230V/255S, 37M/228L, 37M/228L/249S, 37M/38A, 37M/38A/228L, 37M/38V/228L/249K, 37M/38V/249K, 38A/249S/291T, 10F/228L, 10F/228L/249K, 10F/33L/249K, 10F/33L/37M, 10F/33L/37M/38A/249K, 10F/33L/38A/249S, 10F/33L/38V, 10F/33V/249K/251E, 10F/33V/37M, 10F/33V/37M/228L, 10F/33V/37M/249K, 10F/33V/37M/249S, 10F/33V/37M/38A/228L/249K, 10F/33V/37M/38V, 10F/33V/37M/38V/228L/249K, 10F/33V/37M/38V/249K, 10F/249K, 10F/249S, 10F/37M, 10F/37M/228L, 10F/37M/228L/249K, 10F/37M/249K, 10F/37M/38A/228L, 10F/37M/38A/228L/249K, 10F/37M/38V, 10F/37M/38V/228L, 10F/37M/38V/228L/249S, 10F/38A/249K, 10F/38V, 10F/38V/228L, 10F/38V/249S, 77M/255S, 77M/250S, 77M/250S/255S, 77M/226R, 77M/226R/255S, 77M/230V, 77M/230V/255S, 230V/255S, 230V/250S, 230V/250S/255S, 23T/25G/226R/255S, 23T/25G/226R/230V, or 23T/25G/230V, wherein the amino acid positions are relative to the reference sequence corresponding to residues 12 to 346 of SEQ ID NO: 1416, or relative to the reference sequence corresponding to SEQ ID NO: 1416.

In some embodiments, the amino acid sequence of the engineered RNA ligase comprises at least a substitution or substitution set, or amino acid residue(s) M38V, L249K, A255S, Y23K, G33L, A25G, I226R, L249S, E29S, L37M, M38A, S10F, D250S, G75D, T230V, S77M, S251E, Y23T, G33V, F228L, H81Y, Y224A, V64Q, S254K, E268F, P65C, T232N, L332E, L37K, G331R, T330K, A101P, E268G, N307R, Y224Q, D137N, V223C, S280N, V2891, S288E, H342T, V93K, E268M, R296Q, K86G, S241Q, D126M, N277A, K87E, A247I, F243H, I84V, I83A, D192E, V105K, A25G/E29S, A25G/E29S/D250S, A25G/E29S/G75D/S77M/D250S/A255S, A25G/G75D/T230V, A25G/S77M/1226R/A255S, A25G/T230V, D250S/A255S, E29S/G75D/I226R/T230V/A255S, E29S/G75D/S77M, E29S/G75D/S77M/1226R, E29S/G75D/S77M/T230V/A255S, E29S/1226R, E29S/I226R/T230V, E29S/T230V/A255S, G33V/F228L, G33V/L249K, G33V/L249S, G33V/L37M/F228L, G33V/L37M/M38A/F228L, G33V/L37M/M38A/F228L/L249K, G33V/M38V, G75D/D250S/A255S, G75D/1226R, G75D/I226R/D250S/A255S, G75D/I226R/T230V/D250S/A255S, G75D/S77M, G75D/S77M/A255S, G75D/S77M/D250S/A255S, G75D/S77M/I226R/A255S, G75D/S77M/I226R/D250S/A255S, G75D/S77M/T230V, G75D/S77M/T230V/D250S, G75D/T230V, G75D/T230V/A255S, G75D/T230V/D250S, I226R/A255S, I226R/D250S, I226R/T230V, I226R/T230V/A255S, L37M/F228L, L37M/F228L/L249S, L37M/M38A, L37M/M38A/F228L, L37M/M38V/F228L/L249K, L37M/M38V/L249K, M38A/L249S/A291T, S10F/F228L, S10F/F228L/L249K, S10F/G33L/L249K, S10F/G33L/L37M, S10F/G33L/L37M/M38A/L249K, S10F/G33L/M38A/L249S, S10F/G33L/M38V, S10F/G33V/L249K/S251E, S10F/G33V/L37M, S10F/G33V/L37M/F228L, S10F/G33V/L37M/L249K, S10F/G33V/L37M/L249S, S10F/G33V/L37M/M38A/F228L/L249K, S10F/G33V/L37M/M38V, S10F/G33V/L37M/M38V/F228L/L249K, S10F/G33V/L37M/M38V/L249K, S10F/L249K, S10F/L249S, S10F/L37M, S10F/L37M/F228L, S10F/L37M/F228L/L249K, S10F/L37M/L249K, S10F/L37M/M38A/F228L, S10F/L37M/M38A/F228L/L249K, S10F/L37M/M38V, S10F/L37M/M38V/F228L, S10F/L37M/M38V/F228L/L249S, S10F/M38A/L249K, S10F/M38V, S10F/M38V/F228L, S10F/M38V/L249S, S77M/A255S, S77M/D250S, S77M/D250S/A255S, S77M/1226R, S77M/I226R/A255S, S77M/T230V, S77M/T230V/A255S, T230V/A255S, T230V/D250S, T230V/D250S/A255S, Y23T/A25G/1226R/A255S, Y23T/A25G/1226R/T230V, or Y23T/A25G/T230V, wherein the amino acid positions are relative to the reference sequence corresponding to residues 12 to 346 of SEQ ID NO: 1416, or relative to the reference sequence corresponding to SEQ ID NO: 1416.

In some embodiments, the amino acid sequence of the engineered RNA ligase comprises at least a substitution or substitution set at amino acid position(s) 86, 255, 306, 250, 75, 318, 335, 241, 248, 344, 38, 272, 83, 257, 258, or 323, wherein the amino acid positions are relative to the reference sequence corresponding to residues 12 to 346 of SEQ ID NO: 1416, or relative to the reference sequence corresponding to SEQ ID NO: 1416.

In some embodiments, the amino acid sequence of the engineered RNA ligase comprises at least a substitution or substitution set, or amino acid residue(s) 86D, 255V, 306T, 250I, 75A, 318S, 335Q, 241G, 250E, 248F, 344K, 38S, 272S, 83G, 306L, 272M, 248G, 257E, 258M, or 323G, wherein the amino acid positions are relative to the reference sequence corresponding to residues 12 to 346 of SEQ ID NO: 1416, or relative to the reference sequence corresponding to SEQ ID NO: 1416.

In some embodiments, the amino acid sequence of the engineered RNA ligase comprises at least a substitution or substitution set, or amino acid residue(s) K86D, A255V, 1306T, D250I, G75A, F318S, N335Q, S241G, D250E, K248F, V344K, M38S, E272S, I83G, I306L, E272M, K248G, V257E, E258M, or S323G, wherein the amino acid positions are relative to the reference sequence corresponding to residues 12 to 346 of SEQ ID NO: 1416, or relative to the reference sequence corresponding to SEQ ID NO: 1416.

In some embodiments, the engineered RNA ligase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12 to 346 of SEQ ID NO: 1612, or to the reference sequence corresponding to SEQ ID NO: 1612, wherein the amino acid sequence comprises one or more substitutions relative to the reference sequence corresponding to residues 12 to 346 of SEQ ID NO: 1612, or relative to the reference sequence corresponding to SEQ ID NO: 1612.

In some embodiments, the engineered RNA ligase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12 to 346 of an even-numbered SEQ ID NO. of SEQ ID NOs: 1768-1840, or to the reference sequence corresponding to an even-numbered SEQ ID NO. of SEQ ID NOs: 768-1840, wherein the amino acid sequence comprises one or more substitutions relative to the reference sequence corresponding to residues 12 to 346 of SEQ ID NO: 1612, or relative to the reference sequence corresponding to SEQ ID NO: 1612.

In some embodiments, the amino acid sequence of the engineered RNA ligase comprises at least a substitution or substitution set at amino acid position(s) 101, 291, 291/307, 331, 33/75, 33/75/249/291/331, 33/38, 33/93/249, 38/75/291/307, 38/75/249/250, 38/75/93/271, 38/93, 10/75, 10/38/75, 10/38/75/291, 10/38/75/249, 10/38/75/93, 64/224,224, 23/101/307, 23/268, 23/37/64/224/254, 23/64/81, 23/224/268, 23/224/254, 23, 23/64/224, 64, 75/249/307, 93, 33/38/75/249, 75/249, 25/38/75/93/291, 33/38/75, 75, or 23/254, wherein the amino acid positions are relative to the reference sequence corresponding to residues 12 to 346 of SEQ ID NO: 1612, or relative to the reference sequence corresponding to SEQ ID NO: 1612.

In some embodiments, the amino acid sequence of the engineered RNA ligase comprises at least a substitution or substitution set, or amino acid residue(s) 101P, 291T, 291T/307R, 331R, 33V/75D, 33V/75D/249K/291T/331R, 33V/38A, 33V/93K/249K, 38A/75D/291T/307R, 38V/75D/249K/250S, 38V/75D/93K/271G, 38V/93K, 10F/75D, 10F/38A/75D, 10F/38V/75D/291T, 10F/38V/75D/249K, 10F/38V/75D/93K, 64Q/224A, 224A, 224Q, 23T/101P/307K, 23T/268F, 23T/37K/64Q/224A/254K, 23T/64Q/81Y, 23T/224A/268F, 23T/224A/254K, 23T, 23T/64Q/224A, 64Q, 75D/249K/307R, 93K, 33V/38A/75D/249K, 75D/249K, 25S/38V/75D/93K/291T, 33V/38A/75D, 75D, or 23T/254K, wherein the amino acid positions are relative to the reference sequence corresponding to residues 12 to 346 of SEQ ID NO: 1612, or relative to the reference sequence corresponding to SEQ ID NO: 1612.

In some embodiments, the amino acid sequence of the engineered RNA ligase comprises at least a substitution or substitution set, or amino acid residue(s) A101P, A291T, A291T/N307R, G331R, G33V/G75D, G33V/G75D/L249K/A291T/G331R, G33V/M38A, G33V/V93K/L249K, M38A/G75D/A291T/N307R, M38V/G75D/L249K/D250S, M38V/G75D/V93K/S271G, M38V/V93K, S10F/G75D, S10F/M38A/G75D, S10F/M38V/G75D/A291T, S10F/M38V/G75D/L249K, S10F/M38V/G75D/V93K, V64Q/Y224A, Y224A, Y224Q, Y23T/A101P/N307K, Y23T/E268F, Y23T/L37K/V64Q/Y224A/S254K, Y23T/V64Q/H81Y, Y23T/Y224A/E268F, Y23T/Y224A/S254K, Y23T, Y23T/V64Q/Y224A, V64Q, G75D/L249K/N307R, V93K, G33V/M38A/G75D/L249K, G75D/L249K, A25S/M38V/G75D/V93K/A291T, G33V/M38A/G75D, G75D, or Y23T/S254K, wherein the amino acid positions are relative to the reference sequence corresponding to residues 12 to 346 of SEQ ID NO: 1612, or relative to the reference sequence corresponding to SEQ ID NO: 1612.

In some embodiments, the engineered RNA ligase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12 to 346 of SEQ ID NO: 1840, or to the reference sequence corresponding to SEQ ID NO: 1840, wherein the amino acid sequence comprises one or more substitutions relative to the reference sequence corresponding to residues 12 to 346 of SEQ ID NO: 1840, or relative to the reference sequence corresponding to SEQ ID NO: 1840.

In some embodiments, the engineered RNA ligase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12 to 346 of an even-numbered SEQ ID NO. of SEQ ID NOs: 1842-1906, or to the reference sequence corresponding to an even-numbered SEQ ID NO. of SEQ ID NOs: 1842-1906, wherein the amino acid sequence comprises one or more substitutions relative to the reference sequence corresponding to residues 12 to 346 of SEQ ID NO: 1840, or relative to the reference sequence corresponding to SEQ ID NO: 1840.

In some embodiments, the amino acid sequence of the engineered RNA ligase comprises at least a substitution or substitution set at amino acid position(s) 38/65/192, 38/65, 38/65/224, 38/64, 93/307, 38, 38/224, 38/192/224, 65, 192/224, 75/307, 75/250/268/307, 250/268/307/342, 224, 65/192,268, 64/192, 250/268/291/342, 75/93/249/307/342, 250/291, 38/64/192, 38/64/224, 93/249/307, 38/64/86/192/241, 38/342, 248/250/307, 75/77/250/268/307/342, 93/268,192, 38/192, or 64, wherein the amino acid positions are relative to the reference sequence corresponding to residues 12 to 346 of SEQ ID NO: 1840, or relative to the reference sequence corresponding to SEQ ID NO: 1840.

In some embodiments, the amino acid sequence of the engineered RNA ligase comprises at least a substitution or substitution set, or amino acid residue(s) 38A/65C/192E, 38T/65C, 38A/65C/224R, 38A/64Q, 93K/307R, 38A, 38A/224A, 38A/192E/224R, 65C, 38A/192E/224A, 192E/224A, 75D/307R, 75D/250I/268G/307R, 250I/268G/307R/342T, 224R, 65C/192E, 268G, 64Q/192E, 2501/268G/291T/342T, 75D/93K/249K/307R/342T, 250I/291T, 38A/64Q/192E, 38A/64Q/224R, 93K/249K/307R, 38A/64Q/86G/192E/241R, 38A/342T, 192E/224R, 248G/250I/307R, 75D/77M/250I/268G/307R/342T, 93K/268G, 192E, 38A/192E, or 64Q, wherein the amino acid positions are relative to the reference sequence corresponding to residues 12 to 346 of SEQ ID NO: 1840, or relative to the reference sequence corresponding to SEQ ID NO: 1840.

In some embodiments, the amino acid sequence of the engineered RNA ligase comprises at least a substitution or substitution set, or amino acid residue(s) M38A/P65C/D192E, M38T/P65C, M38A/P65C/Y224R, M38A/V64Q, V93K/N307R, M38A, M38A/Y224A, M38A/D192E/Y224R, P65C, M38A/D192E/Y224A, D192E/Y224A, G75D/N307R, G75D/D250I/E268G/N307R, D250I/E268G/N307R/H342T, Y224R, P65C/D192E, E268G, V64Q/D192E, D250I/E268G/A291T/H342T, G75D/V93K/L249K/N307R/H342T, D250I/A291T, M38A/V64Q/D192E, M38A/V64Q/Y224R, V93K/L249K/N307R, M38A/V64Q/K86G/D192E/S241R, M38A/H342T, D192E/Y224R, K248G/D250I/N307R, G75D/S77M/D250I/E268G/N307R/H342T, V93K/E268G, D192E, M38A/D192E, or V64Q, wherein the amino acid positions are relative to the reference sequence corresponding to residues 12 to 346 of SEQ ID NO: 1840, or relative to the reference sequence corresponding to SEQ ID NO: 1840.

In some embodiments, the engineered RNA ligase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12 to 346 of SEQ ID NO: 1866, or to the reference sequence corresponding to SEQ ID NO: 1866, wherein the amino acid sequence comprises one or more substitutions relative to the reference sequence corresponding to residues 12 to 346 of SEQ ID NO: 1866, or relative to the reference sequence corresponding to SEQ ID NO: 1866.

In some embodiments, the engineered RNA ligase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12 to 346 of an even-numbered SEQ ID NO. of SEQ ID NOs: 1948-2038 and 2130-2620, or to the reference sequence corresponding to an even-numbered SEQ ID NO. of SEQ ID NOs: 1948-2038 and 2130-2620, wherein the amino acid sequence comprises one or more substitutions relative to the reference sequence corresponding to residues 12 to 346 of SEQ ID NO: 1866, or relative to the reference sequence corresponding to SEQ ID NO: 1866.

In some embodiments, the amino acid sequence of the engineered RNA ligase comprises at least a substitution or substitution set at amino acid position(s) 38/248/251, 38/248/250/251/277, 248/250/251, 38/93/192/248/251, 38/192/248/251, 250/251, 38/248/250/251, 248/251/344, 192/248/250/251, 38/248/250, 38/93/250/251/277, 38/255/280/318, 248, 38/86/241/255/280/318/335, 38/255, 38, 318, 38/101/255/280, 38/318/335, 38/255/318, 192/250/251/344, 38/335,318/335, 38/277, 38/93/251, 192/251, 255, 38/64/318, 38/64/241/255/318, 251, 64/101/255/280/318, 38/101/255/280/318/335, 38/255/280, 38/251/277, 64/318, 38/64/255/318, 241/255/280/318, 38/86/101/241/318, 38/241, 38/255/318/335, 38/64/101/318, 38/64/255/280/318, 241/255/318, or 38/241/335, wherein the amino acid positions are relative to the reference sequence corresponding to residues 12 to 346 of SEQ ID NO: 1866, or relative to the reference sequence corresponding to SEQ ID NO: 1866.

In some embodiments, the amino acid sequence of the engineered RNA ligase comprises at least a substitution or substitution set, or amino acid residue(s) 38S/248F/251E, 38S/248F/250E/251E/277A, 248F/250E/251E, 38S/93K/192E/248F/251E, 38S/192E/248F/251E, 250E/251E, 38S/248F/250E/251E, 248F/251E/344K, 192E/248F/250E/251R, 38S/248F/250E, 38S/93K/250E/251E/277A, 38A/255V/280N/318S, 248F, 38A/86D/241G/255V/280N/318S/335Q, 38A/255S, 38A, 318S, 38A/101P/255S/280N, 38A/318S/335Q, 38A/255V/318S, 192E/250E/251E/344K, 38A/335Q, 318S/335Q, 38S/277A, 38S/93K/251E, 192E/251E, 255S, 38A/64Q/318S, 2481, 38A/64Q/241G/255V/318S, 251E, 64Q/101P/255V/280N/318S, 38A/101P/255V/280N/318S/335Q, 38A/255V/280N, 38S/251R/277A, 64Q/318S, 38A/64Q/255V/318S, 241G/255S/280N/318S, 255V, 38A/86D/101P/241G/318S, 38A/241G, 38A/255S/318S/335Q, 38A/64Q/101P/318S, 38A/64Q/255V/280N/318S, 241G/255S/318S, or 38A/241G/335Q, wherein the amino acid positions are relative to the reference sequence corresponding to residues 12 to 346 of SEQ ID NO: 1866, or relative to the reference sequence corresponding to SEQ ID NO: 1866.

In some embodiments, the amino acid sequence of the engineered RNA ligase comprises at least a substitution or substitution set, or amino acid residue(s) M38S/K248F/S251E, M38S/K248F/I250E/S251E/N277A, K248F/I250E/S251E, M38S/V93K/D192E/K248F/S251E, M38S/D192E/K248F/S251E, I250E/S251E, M38S/K248F/I250E/S251E, K248F/S251E/V344K, D192E/K248F/I250E/S251R, M38S/K248F/I250E, M38S/V93K/I250E/S251E/N277A, M38A/A255V/S280N/F318S, K248F, M38A/K86D/S241G/A255V/S280N/F318S/N335Q, M38A/A255S, M38A, F318S, M38A/A101P/A255S/S280N, M38A/F318S/N335Q, M38A/A255V/F318S, D192E/I250E/S251E/V344K, M38A/N335Q, F318S/N335Q, M38S/N277A, M38S/V93K/S251E, D192E/S251E, A255S, M38A/V64Q/F318S, K248I, M38A/V64Q/S241G/A255V/F318S, S251E, V64Q/A101P/A255V/S280N/F318S, M38A/A101P/A255V/S280N/F318S/N335Q, M38A/A255V/S280N, M38S/S251R/N277A, V64Q/F318S, M38A/V64Q/A255V/F318S, S241G/A255S/S280N/F318S, A255V, M38A/K86D/A101P/S241G/F318S, M38A/S241G, M38A/A255S/F318S/N335Q, M38A/V64Q/A101P/F318S, M38A/V64Q/A255V/S280N/F318S, S241G/A255S/F318S, or M38A/S241G/N335Q, wherein the amino acid positions are relative to the reference sequence corresponding to residues 12 to 346 of SEQ ID NO: 1866, or relative to the reference sequence corresponding to SEQ ID NO: 1866.

In some embodiments, the amino acid sequence of the engineered RNA ligase comprises at least a substitution or substitution set at amino acid position(s) 89, 122, 181, 28, 125, 157, 238, 330, 212, 143, 208, 290, 161, 214, 84, 270, 91, 295, 316, 286, 301, 170, 264, 289, 190, 183, 128, 60, 310, 131, 302, 267, 288, 185, 291, 129, 280, 298, 158/215, 28/122,276, 274, 271, 215, 294, 106, 58, 135, 45, 299, 315, 204, 332, 169, 305, 154, 158, 297, 95, 337, 324, 150, 179, 283, 206, 236, 266, 213, 336, 178, 99, 184, 282, 57, 177, 109, 191, 107, 160, 144, 56, 159, 341, 325, or 91, wherein the amino acid positions are relative to the reference sequence corresponding to residues 12 to 346 of SEQ ID NO: 1866, or relative to the reference sequence corresponding to SEQ ID NO: 1866.

In some embodiments, the amino acid sequence of the engineered RNA ligase comprises at least a substitution or substitution set or amino acid residue 89D, 89E, 122F, 181P, 28V, 125K, 181R, 157F, 238K, 89Q, 89S, 330E, 122C, 89Y, 89C, 212E, 89W, 143S, 89V, 208P, 290D, 161V, 161M, 214C, 181N, 84V, 270L, 91A, 295V, 122W, 316Y, 286E, 301M, 170Y, 264A, 289D, 289T, 290P, 1901, 316I, 183V, 128E, 289E, 91E, 60V, 310M, 131T, 143F, 301V, 208H, 302S, 60R, 143E, 122Q, 267M, 157L, 288W, 185K, 291Q, 264E, 129W, 280L, 298R, 295K, 122T, 267I, 89G, 286V, 185F, 158R/215G, 295T, 28A/122G, 276M, 274A, 185M, 271T, 215T, 295P, 294V, 270M, 264S, 106L, 301F, 295L, 122L, 106R, 280N, 276G, 310H, 208K, 106Q, 58M, 295Y, 291W, 208T, 135V, 45S, 28L, 299V, 214Y, 295Q, 290N, 302L, 315S, 143C, 89R, 204W, 332W, 169G, 305V, 89L, 154S, 295R, 158R, 135A, 238V, 290H, 294A, 2991, 89N, 288L, 295F, 2971, 125T, 315E, 95C, 337V, 264G, 295S, 289P, 2121, 276A, 60C, 214A, 324D, 185C, 302C, 324M, 301L, 310N, 298C, 295M, 299T, 150L, 238S, 291T, 135M, 179E, 283C, 310S, 206L, 310Q, 106V, 181S, 298K, 299Q, 295C, 143M, 60S, 214H, 212L, 236V, 266C, 289A, 330A, 213N, 302A, 294S, 336I, 178V, 324G, 297L, 991, 184L, 169N, 212G, 282A, 271G, 106M, 271A, 60D, 89T, 3021, 270V, 57V, 143H, 177K, 291L, 214Q, 3011, 271E, 109L, 191C, 213T, 107S, 106W, 185G, 310R, 2891, 282M, 336L, 332H, 291H, 298V, 135H, 280W, 310L, 2661, 135Q, 158S, 288R, 160R, 310T, 286F, 276V, 291R, 330G, 129L, 143T, 212K, 2081, 297A, 286D, 157Q, 301A, 144L, 106G, 283V, 56L, 291S, 264V, 215G, 170H, 294Y, 159F, 286M, 951, 341A, 325L, 214K, 336M, 91V, 337A, 185T, 185S, or 185V, wherein the amino acid positions are relative to the reference sequence corresponding to residues 12 to 346 of SEQ ID NO: 1866, or relative to the reference sequence corresponding to SEQ ID NO: 1866.

In some embodiments, the amino acid sequence of the engineered RNA ligase comprises at least a substitution or substitution set or amino acid residue M89D, M89E, V122F, K181P, I28V, G125K, K181R, V157F, C238K, M89Q, M89S, T330E, V122C, M89Y, M89C, T212E, M89W, V143S, M89V, A208P, T290D, L161V, L161M, L214C, K181N, 184V, F270L, S91A, G295V, V122W, L316Y, I286E, T301M, F170Y, K264A, V289D, V289T, T290P, L190I, L316I, L183V, D128E, V289E, S91E, P60V, A310M, V131T, V143F, T301V, A208H, M302S, P60R, V143E, V122Q, L267M, V157L, S288W, N185K, A291Q, K264E, F129W, S280L, S298R, G295K, V122T, L267I, M89G, I286V, N185F, G158R/A215G, G295T, I28A/V122G, W276M, V274A, N185M, S271T, A215T, G295P, F294V, F270M, K264S, S106L, T301F, G295L, V122L, S106R, S280N, W276G, A310H, A208K, S106Q, 158M, G295Y, A291W, A208T, L135V, A45S, I28L, G299V, L214Y, G295Q, T290N, M302L, D315S, V143C, M89R, F204W, L332W, S169G, I305V, M89L, C154S, G295R, G158R, L135A, C238V, T290H, F294A, G299I, M89N, S288L, G295F, V297I, G125T, D315E, V95C, 1337V, K264G, G295S, V289P, T212I, W276A, P60C, L214A, A324D, N185C, M302C, A324M, T301L, A310N, S298C, G295M, G299T, V150L, C238S, A291T, L135M, D179E, L283C, A310S, P206L, A310Q, S106V, K181S, S298K, G299Q, G295C, V143M, P60S, L214H, T212L, I236V, L266C, V289A, T330A, G213N, M302A, F294S, T336I, N178V, A324G, V297L, L99I, V184L, S169N, T212G, V282A, S271G, S106M, S271A, P60D, M89T, M302I, F270V, 157V, V143H, P177K, A291L, L214Q, T3011, S271E, I109L, V191C, G213T, V107S, S106W, N185G, A310R, V289I, V282M, T336L, L332H, A291H, S298V, L135H, S280W, A310L, L266I, L135Q, G158S, S288R, K160R, A310T, I286F, W276V, A291R, T330G, F129L, V143T, T212K, A208I, V297A, I286D, V157Q, T301A, Y144L, S106G, L283V, V56L, A291S, K264V, A215G, F170H, F294Y, L159F, I286M, V95I, W341A, V325L, L214K, T336M, S91V, 1337A, N185T, N185S, or N185V, wherein the amino acid positions are relative to the reference sequence corresponding to residues 12 to 346 of SEQ ID NO: 1866, or relative to the reference sequence corresponding to SEQ ID NO: 1866.

In some embodiments, the engineered RNA ligase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12 to 346 of SEQ ID NO: 1954, or to the reference sequence corresponding to SEQ ID NO: 1954, wherein the amino acid sequence comprises one or more substitutions relative to the reference sequence corresponding to residues 12 to 346 of SEQ ID NO: 1954, or relative to the reference sequence corresponding to SEQ ID NO: 1954.

In some embodiments, the engineered RNA ligase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12 to 346 of an even-numbered SEQ ID NO. of SEQ ID NOs: 2040-2128, or to the reference sequence corresponding to an even-numbered SEQ ID NO. of SEQ ID NOs: 2040-2128, wherein the amino acid sequence comprises one or more substitutions relative to the reference sequence corresponding to residues 12 to 346 of SEQ ID NO: 1954, or relative to the reference sequence corresponding to SEQ ID NO: 1954.

In some embodiments, the amino acid sequence of the engineered RNA ligase comprises at least a substitution or substitution set at amino acid position(s) 93/232/291/306, 93/232/280/306, 93/241/257/306/335, 232/280, 93/291/306, 93/257, 93/306, 93/280,192/318, 93/280/291, 93/241/291/306, 192, 257/291, 241/257/306, 280/335, 93/291/306/335, 93/335, 93/232/335, 64/318, 93/241, 93/232/241/280, 64/192/318/323, 232/280/306, 93/232,291/335, 257/258, 93/232/241/257/258/280/335, 318/323, 93/241/291/335, 83/86/323, 257/258/291, 101/224/318, 83/192/255/296, 171/232/257/258/280, 83/224/255/318, 64/192/250/318, 101/296, 232/241/280, 64/255/318, 86/250/255, 83/86/101, 83/86, 93/241/258/335, 250/296/323, or 257/258/280, wherein the amino acid positions are relative to the reference sequence corresponding to residues 12 to 346 of SEQ ID NO: 1954, or relative to the reference sequence corresponding to SEQ ID NO: 1954.

In some embodiments, the amino acid sequence of the engineered RNA ligase comprises at least a substitution or substitution set or amino acid residue(s) 93V/232N/291T/306L, 93V/232N/280N/306L, 93V/241G/257E/306L/335Q, 232N/280N, 93V/291T/306L, 93V/257E, 93V/306L, 93V/280N, 192D/318S, 93V/280N/291T, 93V/241G/291T/306L, 192D, 257E/291T, 241G/257E/306L, 280N/335Q, 93V/291T/306L/335Q, 93V/335Q, 93V/232N/335Q, 64Q/318S, 93V/241G, 93V/232N/241G/280N, 64Q/192D/318S/323G, 232N/280N/306L, 93V/232N, 291T/335Q, 257E/258M, 93V/232N/241G/257E/258M/280N/335Q, 318S/323G, 93V/241G/291T/335Q, 83A/86D/323G, 257E/258M/291T, 101P/224A/318S, 83A/192D/255V/296Q, 171S/232N/257E/258M/280N, 83A/224A/255V/318S, 64Q/192D/250E/318S, 101P/296Q, 232N/241G/280N, 64Q/255V/318S, 86D/250E/255V, 83A/86D/101P, 83A/86D, 93V/241G/258M/335Q, 250E/296Q/323G, or 257E/258M/280N, wherein the amino acid positions are relative to the reference sequence corresponding to residues 12 to 346 of SEQ ID NO: 1954, or relative to the reference sequence corresponding to SEQ ID NO: 1954.

In some embodiments, the amino acid sequence of the engineered RNA ligase comprises at least a substitution or substitution set or amino acid residue(s) K93V/T232N/A291T/1306L, K93V/T232N/S280N/1306L, K93V/S241G/V257E/1306L/N335Q, T232N/S280N, K93V/A291T/1306L, K93V/V257E, K93V/I306L, K93V/S280N, E192D/F318S, K93V/S280N/A291T, K93V/S241G/A291T/1306L, E192D, V257E/A291T, S241G/V257E/1306L, S280N/N335Q, K93V/A291T/1306L/N335Q, K93V/N335Q, K93V/T232N/N335Q, V64Q/F318S, K93V/S241G, K93V/T232N/S241G/S280N, V64Q/E192D/F318S/S323G, T232N/S280N/I306L, K93V/T232N, A291T/N335Q, V257E/E258M, K93V/T232N/S241G/V257E/E258M/S280N/N335Q, F318S/S323G, K93V/S241G/A291T/N335Q, I83A/K86D/S323G, V257E/E258M/A291T, A101P/Y224A/F318S, I83A/E192D/A255V/R296Q, A171S/T232N/V257E/E258M/S280N, 183A/Y224A/A255V/F318S, V64Q/E192D/I250E/F318S, A101P/R296Q, T232N/S241G/S280N, V64Q/A255V/F318S, K86D/I250E/A255V, I83A/K86D/A101P, I83A/K86D, K93V/S241G/E258M/N335Q, I250E/R296Q/S323G, or V257E/E258M/S280N, wherein the amino acid positions are relative to the reference sequence corresponding to residues 12 to 346 of SEQ ID NO: 1954, or relative to the reference sequence corresponding to SEQ ID NO: 1954.

In some embodiments, the engineered RNA ligase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12 to 346 of SEQ ID NO: 2062, or to the reference sequence corresponding to SEQ ID NO: 2062, wherein the amino acid sequence comprises one or more substitutions relative to the reference sequence corresponding to residues 12 to 346 of SEQ ID NO: 2062, or relative to the reference sequence corresponding to SEQ ID NO: 2062.

In some embodiments, the engineered RNA ligase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12 to 346 of an even-numbered SEQ ID NO. of SEQ ID NOs: 2622-2746, or to the reference sequence corresponding to an even-numbered SEQ ID NO. of SEQ ID NOs: 2622-2746, wherein the amino acid sequence comprises one or more substitutions relative to the reference sequence corresponding to residues 12 to 346 of SEQ ID NO: 2062, or relative to the reference sequence corresponding to SEQ ID NO: 2062.

In some embodiments, the amino acid sequence of the engineered RNA ligase comprises at least a substitution or substitution set at amino acid position(s) 83/84/330, 91/270/290, 83/181,161/290/330, 181/330, 161/270/330, 270/290, 83/84/270/318/330, 64/83/84/161/192/290/318, 91/161/290/318/330, 161/330, 161/214/270/290/330, 192/270/318, 91/161/192/330, 161/270, 181/270/330, 91/181/192/290, 161/290, 83/161/270, 83/192/318, 214/330, 83/161/290/330, 64/83/161/290/330, 83/161/192/214/318, 122/143/212/295, 161/192/270/290, 181/290, 91/161/214/270/290/318, 91/192/214/270, 214/290, 64/83/161/270/318/330, 91/161/181/330, 161/318/330, 91/270/290/330, 64/181/290/318, 181/270/290/318, 64/83/161/192/270/290/318/330, 64/83/161/214/290/318, 122/192/212, 64/83/91/192/214/330, 83/91/161/181/290/318, 91/161/192/214/270/290, 91/161/181/290/330, 83/91/125/161/270, 28/212,122/212, 28/64/83/122/143/192/212/295/318, 122/295, 91/161/192/318, 28/122/143/212, 64/161/290, 64/122/208/318, 64/83/192/290/318, 64/83/161/290, 181/192/318, 83/161/192/318, 214/270, 83/122, or 192/212, 28/122, wherein the amino acid positions are relative to the reference sequence corresponding to residues 12 to 346 of SEQ ID NO: 2062, or relative to the reference sequence corresponding to SEQ ID NO: 2062.

In some embodiments, the amino acid sequence of the engineered RNA ligase comprises at least a substitution or substitution set or amino acid residue(s) 83A/84V/330E, 91A/270L/290D, 83A/181P, 161V/290D/330E, 181R/330E, 161V/270L/330E, 161M/270L/330E, 270L/290D, 83A/84V/270L/318S/330E, 64Q/83A/84V/161M/192E/290D/318S, 91A/161V/290D/318S/330E, 161V/330E, 161V/214C/270L/290D/330E, 192E/270L/318S, 91A/161M/192E/330E, 161V/270L, 181P/330E, 181P/270L/330E, 91A/181R/192E/290D, 161M/290D, 83A/161M/270L, 83A/192E/318S, 214C/330E, 83A/161M/290D/330E, 64Q/83A/161M/290D/330E, 83A/161M/192E/214C/318S, 122F/143S/212E/295V, 161V/192E/270L/290D, 181P/290D, 91A/161V/214C/270L/290D/318S, 91A/192E/214C/270L, 214C/290D, 64Q/83A/161M/270L/318S/330E, 91A/161M/181P/330E, 161M/318S/330E, 91A/270L/290D/330E, 64Q/181P/290D/318S, 181R/270L/290D/318S, 64Q/83A/161V/192E/270L/290D/318S/330E, 64Q/83A/161M/214C/290D/318S, 122F/192E/212E, 64Q/83A/91A/192E/214C/330E, 83A/91A/161M/181R/290D/318S, 91A/161V/192E/214C/270L/290D, 91A/161V/181P/290D/330E, 83A/91A/125K/161M/270L, 161M/290D/330E, 28V/212E, 122F/212E, 28V/64Q/83A/122F/143S/192E/212E/295V/318S, 122F/295V, 91A/161V/192E/318S, 28V/122F/143S/212E, 64Q/161V/290D, 64Q/122F/208P/318S, 64Q/83A/192E/290D/318S, 64Q/83A/161V/290D, 181R/192E/318S, 83A/161V/192E/318S, 214C/270L, 83A/122F, 192E/212E, or 28V/122F, wherein the amino acid positions are relative to the reference sequence corresponding to residues 12 to 346 of SEQ ID NO: 2062, or relative to the reference sequence corresponding to SEQ ID NO: 2062.

In some embodiments, the amino acid sequence of the engineered RNA ligase comprises at least a substitution or substitution set or amino acid residue(s) 183A/184V/T330E, S91A/F270L/T290D, 183A/K181P, L161V/T290D/T330E, K181R/T330E, L161V/F270L/T330E, L161M/F270L/T330E, F270L/T290D, I83A/I84V/F270L/F318S/T330E, V64Q/183A/I84V/L161M/D192E/T290D/F318S, S91A/L161V/T290D/F318S/T330E, L161V/T330E, L161V/L214C/F270L/T290D/T330E, D192E/F270L/F318S, S91A/L161M/D192E/T330E, L161V/F270L, K181P/T330E, K181P/F270L/T330E, S91A/K181R/D192E/T290D, L161M/T290D, I83A/L161M/F270L, I83A/D192E/F318S, L214C/T330E, I83A/L161M/T290D/T330E, V64Q/I83A/L161M/T290D/T330E, I83A/L161M/D192E/L214C/F318S, V122F/V143S/T212E/G295V, L161V/D192E/F270L/T290D, K181P/T290D, S91A/L161V/L214C/F270L/T290D/F318S, S91A/D192E/L214C/F270L, L214C/T290D, V64Q/183A/L161M/F270L/F318S/T330E, S91A/L161M/K181P/T330E, L161M/F318S/T330E, S91A/F270L/T290D/T330E, V64Q/K181P/T290D/F318S, K181R/F270L/T290D/F318S, V64Q/183A/L161V/D192E/F270L/T290D/F318S/T330E, V64Q/183A/L161M/L214C/T290D/F318S, V122F/D192E/T212E, V64Q/183A/S91A/D192E/L214C/T330E, I83A/S91A/L161M/K181R/T290D/F318S, S91A/L161V/D192E/L214C/F270L/T290D, S91A/L161V/K181P/T290D/T330E, I83A/S91A/G125K/L161M/F270L, L161M/T290D/T330E, I28V/T212E, V122F/T212E, I28V/V64Q/183A/V122F/V143S/D192E/T212E/G295V/F318S, V122F/G295V, S91A/L161V/D192E/F318S, I28V/V122F/V143S/T212E, V64Q/L161V/T290D, V64Q/V122F/A208P/F318S, V64Q/I83A/D192E/T290D/F318S, V64Q/183A/L161V/T290D, K181R/D192E/F318S, I83A/L161V/D192E/F318S, L214C/F270L, 183A/V122F, D192E/T212E, or I28V/V122F, wherein the amino acid positions are relative to the reference sequence corresponding to residues 12 to 346 of SEQ ID NO: 2062, or relative to the reference sequence corresponding to SEQ ID NO: 2062.

In some embodiments, the engineered RNA ligase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12 to 346 of SEQ ID NO: 2674, or to the reference sequence corresponding to SEQ ID NO: 2674, wherein the amino acid sequence comprises one or more substitutions relative to the reference sequence corresponding to residues 12 to 346 of SEQ ID NO: 2674, or relative to the reference sequence corresponding to SEQ ID NO: 2674.

In some embodiments, the engineered RNA ligase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12 to 346 of an even-numbered SEQ ID NO. of SEQ ID NOs: 2748-2782, 2822-2914, and 2916-3012, or to the reference sequence corresponding to an even-numbered SEQ ID NO. of SEQ ID NOs: 2748-2782, 2822-2914, and 2916-3012, wherein the amino acid sequence comprises one or more substitutions relative to the reference sequence corresponding to residues 12 to 346 of SEQ ID NO: 2674, or relative to the reference sequence corresponding to SEQ ID NO: 2674.

In some embodiments, the amino acid sequence of the engineered RNA ligase comprises at least a substitution or substitution set at amino acid position(s) 28/291/295/298/315, 28/95/291/295/298/315, 143/295/298/315, 291/295/298, 28/143/295/298, 28/291/295/298, 143/185/291/295/298, 28/143/185/291/298/336, 28/298,280/295, 143/298/336, 143/291/295/298, 291/295/298/315/336, 143/295/298, 28/143/295/298/336, 95/143/295/298, 143/280/290, or 143/291/298, wherein the amino acid positions are relative to the reference sequence corresponding to residues 12 to 346 of SEQ ID NO: 2674, or relative to the reference sequence corresponding to SEQ ID NO: 2674.

In some embodiments, the amino acid sequence of the engineered RNA ligase comprises at least a substitution or substitution set or amino acid residue(s) 28L/291Q/295G/298V/315E, 28L/951/291S/295G/298V/315E, 143T/295G/298V/315E, 291Q/295G/298V, 28L/143V/295G/298V, 28L/291S/295G/298V, 143V/185V/291Q/295G/298V, 28L/143V/185V/291Q/298V/336I, 28L/298V, 280N/295G, 143V/298V/336I, 143V/291S/295G/298V, 291Q/295G/298V/315E/336I, 143V/295G/298V, 28L/143T/295G/298V/336I, 951/143T/295G/298V, 143V/280N/290N, or 143T/291S/298V, wherein the amino acid positions are relative to the reference sequence corresponding to residues 12 to 346 of SEQ ID NO: 2674, or relative to the reference sequence corresponding to SEQ ID NO: 2674.

In some embodiments, the amino acid sequence of the engineered RNA ligase comprises at least a substitution or substitution set or amino acid residue(s) I28L/A291Q/V295G/S298V/D315E, I28L/V951/A291S/V295G/S298V/D315E, S143T/V295G/S298V/D315E, A291Q/V295G/S298V, I28L/S143V/V295G/S298V, I28L/A291S/V295G/S298V, S143V/N185V/A291Q/V295G/S298V, I28L/S143V/N185V/A291Q/S298V/T336I, I28L/S298V, S280N/V295G, S143V/S298V/T336I, S143V/A291S/V295G/S298V, A291Q/V295G/S298V/D315E/T336I, S143V/V295G/S298V, I28L/S143T/V295G/S298V/T336I, V95I/S143T/V295G/S298V, S143V/S280N/T290N, or S143T/A291S/S298V, wherein the amino acid positions are relative to the reference sequence corresponding to residues 12 to 346 of SEQ ID NO: 2674, or relative to the reference sequence corresponding to SEQ ID NO: 2674.

In some embodiments, the amino acid sequence of the engineered RNA ligase comprises at least a substitution or substitution set at amino acid position(s) 208, 25, 90, 141, 258, 248, 318, 117, 299, 33, 80, 253, 83, 254, 256, 30, 327, 73, 100, 97, 119, 265, 296, 32, 156, 40, 105, 107, 41/114, 41, or 64, wherein the amino acid positions are relative to the reference sequence corresponding to residues 12 to 346 of SEQ ID NO: 2674, or relative to the reference sequence corresponding to SEQ ID NO: 2674.

In some embodiments, the amino acid sequence of the engineered RNA ligase comprises at least a substitution or substitution set or amino acid residue 208T, 25G, 90E, 141D, 258D, 248G, 248K, 318E, 318S, 117Q, 299K, 33I, 33T, 80E, 253W, 83E, 83T, 254Q, 254V, 256A, 256G, 256M, 256W, 30L, 30V, 30W, 327E, 73Q, 73W, 100A, 100G, 100H, 100S, 97A, 119L, 265A, 296E, 296L, 296Q, 32E, 156Q, 40I, 105L, 107M, 41E/A114E, 41I, or 64A, wherein the amino acid positions are relative to the reference sequence corresponding to residues 12 to 346 of SEQ ID NO: 2674, or relative to the reference sequence corresponding to SEQ ID NO: 2674.

In some embodiments, the amino acid sequence of the engineered RNA ligase comprises at least a substitution or substitution set or amino acid residue A208T, A25G, D90E, E141D, E258D, F248G, F248K, F318E, F318S, G117Q, G299K, G33I, G33T, G80E, I253W, I83E, 183T, K254Q, K254V, K256A, K256G, K256M, K256W, K30L, K30V, K30W, K327E, L73Q, L73W, N100A, N100G, N100H, N100S, N97A, Q119L, R265A, R296E, R296L, R296Q, R32E, T156Q, T40I, V105L, V107M, V41E/A114E, V41I, or V64A, wherein the amino acid positions are relative to the reference sequence corresponding to residues 12 to 346 of SEQ ID NO: 2674, or relative to the reference sequence corresponding to SEQ ID NO: 2674.

In some embodiments, the amino acid sequence of the engineered RNA ligase comprises at least a substitution or substitution set at amino acid position(s) 162, 208, 25, 103, 346, 42, 74, 113, 13, 228, 318, 121, 299, 33, 342, 253, 127, 254, 292, 30, 67, 86, 165, 173, 300, 317, 326, 11, 229, 97, 71, 17, 210, 241, 271, 40, 56, or 95, wherein the amino acid positions are relative to the reference sequence corresponding to residues 12 to 346 of SEQ ID NO: 2674, or relative to the reference sequence corresponding to SEQ ID NO: 2674.

In some embodiments, the amino acid sequence of the engineered RNA ligase comprises at least a substitution or substitution set or amino acid residue 162C, 208L, 25D, 25F, 25T, 103V, 346S, 42T, 74D, 113M, 13W, 228D, 228L, 318A, 318G, 121N, 299E, 299R, 33L, 342S, 253L, 127P, 254E, 254N, 292N, 292S, 30A, 30T, 67R, 86L, 165M, 173M, 300A, 317E, 317Q, 317V, 326C, 11G, 229W, 97E, 71T, 17P, 210V, 241E, 271G, 40P, 40S, 56I, or 95A, wherein the amino acid positions are relative to the reference sequence corresponding to residues 12 to 346 of SEQ ID NO: 2674, or relative to the reference sequence corresponding to SEQ ID NO: 2674.

In some embodiments, the amino acid sequence of the engineered RNA ligase comprises at least a substitution or substitution set or amino acid residue A162C, A208L, A25D, A25F, A25T, D103V, E346S, E42T, E74D, F113M, F13W, F228D, F228L, F318A, F318G, G121N, G299E, G299R, G33L, H342S, I253L, K127P, K254E, K254N, K292N, K292S, K30A, K30T, K67R, K86L, L165M, L173M, L300A, L317E, L317Q, L317V, L326C, N11G, N229W, N97E, P71T, S17P, S210V, S241E, S271G, T40P, T40S, V56I, or V95A, wherein the amino acid positions are relative to the reference sequence corresponding to residues 12 to 346 of SEQ ID NO: 2674, or relative to the reference sequence corresponding to SEQ ID NO: 2674.

In some embodiments, the engineered RNA ligase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12 to 346 of SEQ ID NO: 2754, or to the reference sequence corresponding to SEQ ID NO: 2754, wherein the amino acid sequence comprises one or more substitutions relative to the reference sequence corresponding to residues 12 to 346 of SEQ ID NO: 2754, or relative to the reference sequence corresponding to SEQ ID NO: 2754.

In some embodiments, the engineered RNA ligase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12 to 346 of an even-numbered SEQ ID NO. of SEQ ID NOs: 2784-2820, or to the reference sequence corresponding to an even-numbered SEQ ID NO. of SEQ ID NOs: 2784-2820, wherein the amino acid sequence comprises one or more substitutions relative to the reference sequence corresponding to residues 12 to 346 of SEQ ID NO: 2754, or relative to the reference sequence corresponding to SEQ ID NO: 2754.

In some embodiments, the amino acid sequence of the engineered RNA ligase comprises at least a substitution or substitution set at amino acid position(s) 106/264/280, 280/330, 28/106/264/291, 28/264/280/330, 57/60/135/264/291, 28/135/264/280, 106/264/280/291, 28/106/264/280, 280/299, 28/57/89/106/264/280/291, 264, 185/291, 28/106/299, 28/214/264/299, 264/330, 28/89/214/291, 28/185/271/310, 122/271, or 89/106/135/264/280/330, wherein the amino acid positions are relative to the reference sequence corresponding to residues 12 to 346 of SEQ ID NO: 2754, or relative to the reference sequence corresponding to SEQ ID NO: 2754.

In some embodiments, the amino acid sequence of the engineered RNA ligase comprises at least a substitution or substitution set or amino acid residue(s) 106R/264E/280L, 280L/330A, 28L/106M/264S/291S, 28L/264E/280L/330A, 57V/60R/135V/264S/291S, 28L/135V/264S/280L, 106L/264E/280L/291S, 28L/106L/264S/280L, 280L/2991, 28L/57V/89G/106L/264S/280L/291S, 264S, 185S/291S, 28L/106M/299V, 28L/214Y/264S/299V, 264E/330A, 28V/89N/214Q/291S, 28V/185K/271G/310H, 122T/271G, or 89N/106M/135V/264E/280L/330A, wherein the amino acid positions are relative to the reference sequence corresponding to residues 12 to 346 of SEQ ID NO: 2754, or relative to the reference sequence corresponding to SEQ ID NO: 2754.

In some embodiments, the amino acid sequence of the engineered RNA ligase comprises at least a substitution or substitution set or amino acid residue(s) S106R/K264E/S280L, S280L/T330A, I28L/S106M/K264S/Q291S, I28L/K264E/S280L/T330A, I57V/P60R/L135V/K264S/Q291S, I28L/L135V/K264S/S280L, S106L/K264E/S280L/Q291S, I28L/S106L/K264S/S280L, S280L/G299I, I28L/I57V/M89G/S106L/K264S/S280L/Q291S, K264S, N185S/Q291S, I28L/S106M/G299V, I28L/L214Y/K264S/G299V, K264E/T330A, I28V/M89N/L214Q/Q291S, I28V/N185K/S271G/A310H, F122T/S271G, M89N/S106M/L135V/K264E/S280L/T330A, wherein the amino acid positions are relative to the reference sequence corresponding to residues 12 to 346 of SEQ ID NO: 2754, or relative to the reference sequence corresponding to SEQ ID NO: 2754.

In some embodiments, the engineered RNA ligase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12 to 346 of SEQ ID NO: 2786, or to the reference sequence corresponding to SEQ ID NO: 2786, wherein the amino acid sequence comprises one or more substitutions relative to the reference sequence corresponding to residues 12 to 346 of SEQ ID NO: 2786, or relative to the reference sequence corresponding to SEQ ID NO: 2786.

In some embodiments, the engineered RNA ligase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12 to 346 of an even-numbered SEQ ID NO. of SEQ ID NOs: 3014-3058, or to the reference sequence corresponding to an even-numbered SEQ ID NO. of SEQ ID NOs: 3014-3058, wherein the amino acid sequence comprises one or more substitutions relative to the reference sequence corresponding to residues 12 to 346 of SEQ ID NO: 2786, or relative to the reference sequence corresponding to SEQ ID NO: 2786.

In some embodiments, the amino acid sequence of the engineered RNA ligase comprises at least a substitution or substitution set at amino acid position(s) 25/228/248/264, 32/113/228/248, 25/32/113/241/248/264/299, 25/113/248/264/318, 25/127/228/264/299/318, 25/113/228/248/327, 228/241/264, 264/299/318/327, 25/113/241/248/264/318, 25/127/248/299/318, 113/127/228/264/299, 25/127, 25/228/248/264/327, 25/113/264/299, 32/113/264/318, 25/32/241/264/327, 113/264/327, 25/127/228/248/318, 327, 32/113/254/264, 25/228/241/264, 25/113/228/241/318, or 113/327, wherein the amino acid positions are relative to the reference sequence corresponding to residues 12 to 346 of SEQ ID NO: 2786, or relative to the reference sequence corresponding to SEQ ID NO: 2786.

In some embodiments, the amino acid sequence of the engineered RNA ligase comprises at least a substitution or substitution set or amino acid residue(s) 25D/228L/248G/264S, 32E/113M/228L/248G, 25D/32E/113L/241E/248G/264S/299E, 25D/113M/248G/264E/318E, 25D/127P/228L/264S/299E/318E, 25D/113M/228L/248G/327E, 228L/241E/264E, 264S/299E/318E/327E, 25D/113M/241E/248G/264S/318E, 25D/127P/248G/299E/318E, 113M/127P/228L/264E/299E, 25D/127P, 25D/228D/248G/264E/327E, 25D/113M/264E/299E, 32E/113M/264S/318E, 25D/32E/241E/264S/327E, 113M/264E/327E, 25D/127P/228D/248G/318E, 327E, 32E/113M/254E/264E, 25D/228L/241E/264E, 25D/113M/228L/241E/318E, of 113M/327E, wherein the amino acid positions are relative to the reference sequence corresponding to residues 12 to 346 of SEQ ID NO: 2786, or relative to the reference sequence corresponding to SEQ ID NO: 2786.

In some embodiments, the amino acid sequence of the engineered RNA ligase comprises at least a substitution or substitution set or amino acid residue(s) A25D/F228L/F248G/K264S, R32E/F113M/F228L/F248G, A25D/R32E/F113L/S241E/F248G/K264S/G299E, A25D/F113M/F248G/K264E/F318E, A25D/K127P/F228L/K264S/G299E/F318E, A25D/F113M/F228L/F248G/K327E, F228L/S241E/K264E, K264S/G299E/F318E/K327E, A25D/F113M/S241E/F248G/K264S/F318E, A25D/K127P/F248G/G299E/F318E, F113M/K127P/F228L/K264E/G299E, A25D/K127P, A25D/F228D/F248G/K264E/K327E, A25D/F113M/K264E/G299E, R32E/F113M/K264S/F318E, A25D/R32E/S241E/K264S/K327E, F113M/K264E/K327E, A25D/K127P/F228D/F248G/F318E, K327E, R32E/F113M/K254E/K264E, A25D/F228L/S241E/K264E, A25D/F113M/F228L/S241E/F318E, or F113M/K327E, wherein the amino acid positions are relative to the reference sequence corresponding to residues 12 to 346 of SEQ ID NO: 2786, or relative to the reference sequence corresponding to SEQ ID NO: 2786.

In some embodiments, the amino acid sequence of the engineered RNA ligase comprises at least a substitution at an amino acid position provided in Tables 6.1, 6.2, 7.1, 8.1, 8.2, 9.1, 10.1, 11.1, 12.1, 13.1, 14.1, 15.1, 16.1, 17.1, 18.1, 19.1, 20.1, 21.1, 22.1, 23.1, 24.1, 25.1, and 26.1, wherein the amino acid positions are relative to the reference sequence corresponding to residues 12 to 346 of SEQ ID NO: 54, 244, 648, 862, 956, 1088, 1416, 1612, 1840, 1866, 1954, 2062, 2674, 2754, or 2786, or relative to the reference sequence corresponding to SEQ ID NO: 54, 244, 648, 862, 956, 1088, 1416, 1612, 1840, 1866, 1954, 2062, 2674, 2754, or 2786.

In some embodiments, the amino acid sequence of the engineered RNA ligase comprises at least one substitution provided in Tables 6.1, 6.2, 7.1, 8.1, 8.2, 9.1, 10.1, 11.1, 12.1, 13.1, 14.1, 15.1, 16.1, 17.1, 18.1, 19.1, 20.1, 21.1, 22.1, 23.1, 24.1, 25.1, and 26.1, wherein the amino acid positions are relative to the reference sequence corresponding to residues 12 to 346 of SEQ ID NO: 54, 244, 648, 862, 956, 1088, 1416, 1612, 1840, 1866, 1954, 2062, 2674, 2754, or 2786, or relative to the reference sequence corresponding to SEQ ID NO: 54, 244, 648, 862, 956, 1088, 1416, 1612, 1840, 1866, 1954, 2062, 2674, 2754, or 2786.

In some embodiments, the amino acid sequence of the engineered RNA ligase comprises at least a substitution or substitution set at the amino acid position(s) provided in Tables 6.1, 6.2, 7.1, 8.1, 8.2, 9.1, 10.1, 11.1, 12.1, 13.1, 14.1, 15.1, 16.1, 17.1, 18.1, 19.1, 20.1, 21.1, 22.1, 23.1, 24.1, 25.1, and 26.1, wherein the amino acid positions are relative to the reference sequence corresponding to residues 12 to 346 of SEQ ID NO: 54, 244, 648, 862, 956, 1088, 1416, 1612, 1840, 1866, 1954, 2062, 2674, 2754, or 2786, or relative to the reference sequence corresponding to SEQ ID NO: 54, 244, 648, 862, 956, 1088, 1416, 1612, 1840, 1866, 1954, 2062, 2674, 2754, or 2786.

In some embodiments, the amino acid sequence of the engineered RNA ligase comprises at least a substitution or substitution set of an RNA ligase variant provided in Tables 6.1, 6.2, 7.1, 8.1, 8.2, 9.1, 10.1, 11.1, 12.1, 13.1, 14.1, 15.1, 16.1, 17.1, 18.1, 19.1, 20.1, 21.1, 22.1, 23.1, 24.1, 25.1, and 26.1, wherein the amino acid positions are relative to the reference sequence corresponding to residues 12 to 346 of SEQ ID NO: 54, 244, 648, 862, 956, 1088, 1416, 1612, 1840, 1866, 1954, 2062, 2674, 2754, or 2786, or relative to the reference sequence corresponding to SEQ ID NO: 54, 244, 648, 862, 956, 1088, 1416, 1612, 1840, 1866, 1954, 2062, 2674, 2754, or 2786.

In some embodiments, the engineered RNA ligase comprises an amino acid sequence having least 70%, 75%, 80%, 81%, 82%, 83%, 84%, 85%, 86%, 87%, 88%, 89%, 90%, 91%, 92%, 93%, 94%, 95%, 96%, 97%, 98%, 99% or more sequence identity to a reference sequence comprising a substitution or substitution set provided in Tables 6.1, 6.2, 7.1, 8.1, 8.2, 9.1, 10.1, 11.1, 12.1, 13.1, 14.1, 15.1, 16.1, 17.1, 18.1, 19.1, 20.1, 21.1, 22.1, 23.1, 24.1, 25.1, and 26.1, wherein the amino acid positions are relative to the reference sequence corresponding to residues 12 to 346 of SEQ ID NO: 54, 244, 648, 862, 956, 1088, 1416, 1612, 1840, 1866, 1954, 2062, 2674, 2754, or 2786, or relative to the reference sequence corresponding to SEQ ID NO: 54, 244, 648, 862, 956, 1088, 1416, 1612, 1840, 1866, 1954, 2062, 2674, 2754, or 2786.

In some embodiments, the engineered RNA ligase comprises an amino acid sequence having least 70%, 75%, 80%, 81%, 82%, 83%, 84%, 85%, 86%, 87%, 88%, 89%, 90%, 91%, 92%, 93%, 94%, 95%, 96%, 97%, 98%, 99% or more sequence identity to the sequence comprising residues 12 to 346 of an engineered RNA ligase set forth in Tables 6.1, 6.2, 7.1, 8.1, 8.2, 9.1, 10.1, 11.1, 12.1, 13.1, 14.1, 15.1, 16.1, 17.1, 18.1, 19.1, 20.1, 21.1, 22.1, 23.1, 24.1, 25.1, and 26.1, or to the sequence of an engineered RNA ligase set forth in Tables 6.1, 6.2, 7.1, 8.1, 8.2, 9.1, 10.1, 11.1, 12.1, 13.1, 14.1, 15.1, 16.1, 17.1, 18.1, 19.1, 20.1, 21.1, 22.1, 23.1, 24.1, 25.1, and 26.1.

In some embodiments, the engineered RNA ligase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99% or more sequence identity to the sequence corresponding to residues 12 to 346 of SEQ ID NO: 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 322, 324, 326, 328, 330, 332, 334, 336, 338, 340, 342, 344, 346, 348, 350, 352, 354, 356, 358, 360, 362, 364, 366, 368, 370, 372, 374, 376, 378, 380, 382, 384, 386, 388, 390, 392, 394, 396, 398, 400, 402, 404, 406, 408, 410, 412, 414, 416, 418, 420, 422, 424, 426, 428, 430, 432, 434, 436, 438, 440, 442, 444, 446, 448, 450, 452, 454, 456, 458, 460, 462, 464, 466, 468, 470, 472, 474, 476, 478, 480, 482, 484, 486, 488, 490, 492, 494, 496, 498, 500, 502, 504, 506, 508, 510, 512, 514, 516, 518, 520, 522, 524, 526, 528, 530, 532, 534, 536, 538, 540, 542, 544, 546, 548, 550, 552, 554, 556, 558, 560, 562, 564, 566, 568, 570, 572, 574, 576, 578, 580, 582, 584, 586, 588, 590, 592, 594, 596, 598, 600, 602, 604, 606, 608, 610, 612, 614, 616, 618, 620, 622, 624, 626, 628, 630, 632, 634, 636, 638, 640, 642, 644, 646, 648, 650, 652, 654, 656, 658, 660, 662, 664, 666, 668, 670, 672, 674, 676, 678, 680, 682, 684, 686, 688, 690, 692, 694, 696, 698, 700, 702, 704, 706, 708, 710, 712, 714, 716, 718, 720, 722, 724, 726, 728, 730, 732, 734, 736, 738, 740, 742, 744, 746, 748, 750, 752, 754, 756, 758, 760, 762, 764, 766, 768, 770, 772, 774, 776, 778, 780, 782, 784, 786, 788, 790, 792, 794, 796, 798, 800, 802, 804, 806, 808, 810, 812, 814, 816, 818, 820, 822, 824, 826, 828, 830, 832, 834, 836, 838, 840, 842, 844, 846, 848, 850, 852, 854, 856, 858, 860, 862, 864, 866, 868, 870, 872, 874, 876, 878, 880, 882, 884, 886, 888, 890, 892, 894, 896, 898, 900, 902, 904, 906, 908, 910, 912, 914, 916, 918, 920, 922, 924, 926, 928, 930, 932, 934, 936, 938, 940, 942, 944, 946, 948, 950, 952, 954, 956, 958, 960, 962, 964, 966, 968, 970, 972, 974, 976, 978, 980, 982, 984, 986, 988, 990, 992, 994, 996, 998, 1000, 1002, 1004, 1006, 1008, 1010, 1012, 1014, 1016, 1018, 1020, 1022, 1024, 1026, 1028, 1030, 1032, 1034, 1036, 1038, 1040, 1042, 1044, 1046, 1048, 1050, 1052, 1054, 1056, 1058, 1060, 1062, 1064, 1066, 1068, 1070, 1072, 1074, 1076, 1078, 1080, 1082, 1084, 1086, 1088, 1090, 1092, 1094, 1096, 1098, 1100, 1102, 1104, 1106, 1108, 1110, 1112, 1114, 1116, 1118, 1120, 1122, 1124, 1126, 1128, 1130, 1132, 1134, 1136, 1138, 1140, 1142, 1144, 1146, 1148, 1150, 1152, 1154, 1156, 1158, 1160, 1162, 1164, 1166, 1168, 1170, 1172, 1174, 1176, 1178, 1180, 1182, 1184, 1186, 1188, 1190, 1192, 1194, 1196, 1198, 1200, 1202, 1204, 1206, 1208, 1210, 1212, 1214, 1216, 1218, 1220, 1222, 1224, 1226, 1228, 1230, 1232, 1234, 1236, 1238, 1240, 1242, 1244, 1246, 1248, 1250, 1252, 1254, 1256, 1258, 1260, 1262, 1264, 1266, 1268, 1270, 1272, 1274, 1276, 1278, 1280, 1282, 1284, 1286, 1288, 1290, 1292, 1294, 1296, 1298, 1300, 1302, 1304, 1306, 1308, 1310, 1312, 1314, 1316, 1318, 1320, 1322, 1324, 1326, 1328, 1330, 1332, 1334, 1336, 1338, 1340, 1342, 1344, 1346, 1348, 1350, 1352, 1354, 1356, 1358, 1360, 1362, 1364, 1366, 1368, 1370, 1372, 1374, 1376, 1378, 1380, 1382, 1384, 1386, 1388, 1390, 1392, 1394, 1396, 1398, 1400, 1402, 1404, 1406, 1408, 1410, 1412, 1414, 1416, 1418, 1420, 1422, 1424, 1426, 1428, 1430, 1432, 1434, 1436, 1438, 1440, 1442, 1444, 1446, 1448, 1450, 1452, 1454, 1456, 1458, 1460, 1462, 1464, 1466, 1468, 1470, 1478, 1480, 1482, 1484, 1486, 1488, 1490, 1492, 1494, 1496, 1498, 1500, 1502, 1504, 1506, 1508, 1510, 1512, 1514, 1516, 1518, 1520, 1522, 1524, 1526, 1528, 1530, 1532, 1534, 1536, 1538, 1540, 1542, 1544, 1546, 1548, 1550, 1552, 1554, 1556, 1558, 1560, 1562, 1564, 1566, 1568, 1570, 1572, 1574, 1576, 1578, 1580, 1582, 1584, 1586, 1588, 1590, 1592, 1594, 1596, 1598, 1600, 1602, 1604, 1606, 1608, 1610, 1612, 1614, 1616, 1618, 1620, 1622, 1624, 1626, 1628, 1630, 1632, 1634, 1636, 1638, 1640, 1642, 1644, 1646, 1648, 1650, 1652, 1654, 1656, 1658, 1660, 1662, 1664, 1666, 1668, 1670, 1672, 1674, 1676, 1678, 1680, 1682, 1684, 1686, 1688, 1690, 1692, 1694, 1696, 1698, 1700, 1702, 1704, 1706, 1708, 1710, 1712, 1714, 1716, 1718, 1720, 1722, 1724, 1726, 1728, 1730, 1732, 1734, 1736, 1738, 1740, 1742, 1744, 1746, 1748, 1750, 1752, 1754, 1756, 1758, 1760, 1762, 1764, 1766, 1768, 1770, 1772, 1774, 1776, 1778, 1780, 1782, 1784, 1786, 1788, 1790, 1792, 1794, 1796, 1798, 1800, 1802, 1804, 1806, 1808, 1810, 1812, 1814, 1816, 1818, 1820, 1822, 1824, 1826, 1828, 1830, 1832, 1834, 1836, 1838, 1840, 1842, 1844, 1846, 1848, 1850, 1852, 1854, 1856, 1858, 1860, 1862, 1864, 1866, 1868, 1870, 1872, 1874, 1876, 1878, 1880, 1882, 1884, 1886, 1888, 1890, 1892, 1894, 1896, 1898, 1900, 1902, 1904, 1906, 1908, 1910, 1912, 1914, 1916, 1918, 1920, 1922, 1924, 1926, 1928, 1930, 1932, 1934, 1936, 1938, 1940, 1942, 1944, 1946, 1948, 1950, 1952, 1954, 1956, 1958, 1960, 1962, 1964, 1966, 1968, 1970, 1972, 1974, 1976, 1978, 1980, 1982, 1984, 1986, 1988, 1990, 1992, 1994, 1996, 1998, 2000, 2002. 2004, 2006, 2008, 2010, 2012, 2014, 2016, 2018, 2020, 2022, 2024, 2026, 2028, 2030, 2032, 2034, 2036, 2038, 2040, 2042, 2044, 2046, 2048, 2050, 2052, 2054, 2056, 2058, 2060, 2062, 2064, 2066, 2068, 2070, 2072, 2074, 2076, 2078, 2080, 2082, 2084, 2086, 2088, 2090, 2092, 2094, 2096, 2098, 2100, 2102, 2104, 2106, 2108, 2110, 2112, 2114, 2116, 2118, 2120, 2122, 2124, 2126, 2128, 2130, 2132, 2134, 2136, 2138, 2140, 2142, 2144, 2146, 2148, 2150, 2152, 2154, 2156, 2158, 2160, 2162, 2164, 2166, 2168, 2170, 2172, 2174, 2176, 2178, 2180, 2182, 2184, 2186, 2188, 2190, 2192, 2194, 2196, 2198, 2200, 2202, 2204, 2206, 2208, 2210, 2212, 2214, 2216, 2218, 2220, 2222, 2224, 2226, 2228, 2230, 2232, 2234, 2236, 2238, 2240, 2242, 2244, 2246, 2248, 2250, 2252, 2254, 2256, 2258, 2260, 2262, 2264, 2266, 2268, 2270, 2272, 2274, 2276, 2278, 2280, 2282, 2284, 2286, 2288, 2290, 2292, 2294, 2296, 2298, 2300, 2302, 2304, 2306, 2308, 2310, 2312, 2314, 2316, 2318, 2320, 2322, 2324, 2326, 2328, 2330, 2332, 2334, 2336, 2338, 2340, 2342, 2344, 2346, 2348, 2350, 2352, 2354, 2356, 2358, 2360, 2362, 2364, 2366, 2368, 2370, 2372, 2374, 2376, 2378, 2380, 2382, 2384, 2386, 2388, 2390, 2392, 2394, 2396, 2398, 2400, 2402, 2404, 2406, 2408, 2410, 2412, 2414, 2416, 2418, 2420, 2422, 2424, 2426, 2428, 2430, 2432, 2434, 2436, 2438, 2440, 2442, 2444, 2446, 2448, 2450, 2452, 2454, 2456, 2458, 2460, 2462, 2464, 2466, 2468, 2470, 2472, 2474, 2476, 2478, 2480, 2482, 2484, 2486, 2488, 2490, 2492, 2494, 2496, 2498, 2500, 2502, 2504, 2506, 2508, 2510, 2512, 2514, 2516, 2518, 2520, 2522, 2524, 2526, 2528, 2530, 2532, 2534, 2536, 2538, 2540, 2542, 2544, 2546, 2548, 2550, 2552, 2554, 2556, 2558, 2560, 2562, 2564, 2566, 2568, 2570, 2572, 2574, 2576, 2578, 2580, 2582, 2584, 2586, 2588, 2590, 2592, 2594, 2596, 2598, 2600, 2602, 2604, 2606, 2608, 2610, 2612, 2614, 2616, 2618, 2620, 2622, 2624, 2626, 2628, 2630, 2632, 2634, 2636, 2638, 2640, 2642, 2644, 2646, 2648, 2650, 2652, 2654, 2656, 2658, 2660, 2662, 2664, 2666, 2668, 2670, 2672, 2674, 2676, 2678, 2680, 2682, 2684, 2686, 2688, 2690, 2692, 2694, 2696, 2698, 2700, 2702, 2704, 2706, 2708, 2710, 2712, 2714, 2716, 2718, 2720, 2722, 2724, 2726, 2728, 2730, 2732, 2734, 2736, 2738, 2740, 2742, 2744, 2746, 2748, 2750, 2752, 2754, 2756, 2758, 2760, 2762, 2764, 2766, 2768, 2770, 2772, 2774, 2776, 2778, 2780, 2782, 2784, 2786, 2788, 2790, 2792, 2794, 2796, 2798, 2800, 2802, 2804, 2806, 28208, 2810, 2812, 2814, 2816, 2818, 2820, 2822, 2824, 2826, 2828, 2830, 2832, 2834, 2836, 2838, 2840, 2842, 2844, 2846, 2848, 2850, 2852, 2854, 2856, 2858, 2860, 2862, 2864, 2866, 2868, 2870, 2872, 2874, 2876, 2878, 2880, 2882, 2884, 2886, 2888, 2890, 2892, 2894, 2896, 2898, 2900, 2902, 2904, 2906, 2908, 2910, 2912, 2914, 2916, 2918, 2920, 2922, 2924, 2926, 2928, 2930, 2932, 2934, 2936, 2938, 2940, 2942, 2944, 2946, 2948, 2950, 2952, 2954, 2956, 2958, 2960, 2962, 2964, 2966, 2968, 2970, 2972, 2974, 2976, 2978, 2980, 2982, 2984, 2986, 2988, 2990, 2992, 2994, 2996, 2998, 3000, 3002, 3004, 3006, 3008, 3010, 3012, 3014, 3016, 3018, 3020, 3022, 3024, 3026, 3028, 3030, 3032, 3034, 3036, 3038, 3040, 3042, 3044, 3046, 3048, 3050, 3052, 3054, 3056, or 3058.

In some embodiments, the engineered RNA ligase comprises an amino acid sequence having at least 70%, 75%, 80%, 85%, 86%, 87%, 88%, 89%, 90%, 91%, 92%, 93%, 94%, 95%, 96%, 97%, 98%, 99% or more sequence identity to the sequence corresponding to SEQ ID NO: 4, 6, 8, 10, 12, 14, 16, 18, 20, 22, 24, 26, 28, 30, 32, 34, 336, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 322, 324, 326, 328, 330, 332, 334, 336, 338, 340, 342, 344, 346, 348, 350, 352, 354, 356, 358, 360, 362, 364, 366, 368, 370, 372, 374, 376, 378, 380, 382, 384, 386, 388, 390, 392, 394, 396, 398, 400, 402, 404, 406, 408, 410, 412, 414, 416, 418, 420, 422, 424, 426, 428, 430, 432, 434, 436, 438, 440, 442, 444, 446, 448, 450, 452, 454, 456, 458, 460, 462, 464, 466, 468, 470, 472, 474, 476, 478, 480, 482, 484, 486, 488, 490, 492, 494, 496, 498, 500, 502, 504, 506, 508, 510, 512, 514, 516, 518, 520, 522, 524, 526, 528, 530, 532, 534, 536, 538, 540, 542, 544, 546, 548, 550, 552, 554, 556, 558, 560, 562, 564, 566, 568, 570, 572, 574, 576, 578, 580, 582, 584, 586, 588, 590, 592, 594, 596, 598, 600, 602, 604, 606, 608, 610, 612, 614, 616, 618, 620, 622, 624, 626, 628, 630, 632, 634, 636, 638, 640, 642, 644, 646, 648, 650, 652, 654, 656, 658, 660, 662, 664, 666, 668, 670, 672, 674, 676, 678, 680, 682, 684, 686, 688, 690, 692, 694, 696, 698, 700, 702, 704, 706, 708, 710, 712, 714, 716, 718, 720, 722, 724, 726, 728, 730, 732, 734, 736, 738, 740, 742, 744, 746, 748, 750, 752, 754, 756, 758, 760, 762, 764, 766, 768, 770, 772, 774, 776, 778, 780, 782, 784, 786, 788, 790, 792, 794, 796, 798, 800, 802, 804, 806, 808, 810, 812, 814, 816, 818, 820, 822, 824, 826, 828, 830, 832, 834, 836, 838, 840, 842, 844, 846, 848, 850, 852, 854, 856, 858, 860, 862, 864, 866, 868, 870, 872, 874, 876, 878, 880, 882, 884, 886, 888, 890, 892, 894, 896, 898, 900, 902, 904, 906, 908, 910, 912, 914, 916, 918, 920, 922, 924, 926, 928, 930, 932, 934, 936, 938, 940, 942, 944, 946, 948, 950, 952, 954, 956, 958, 960, 962, 964, 966, 968, 970, 972, 974, 976, 978, 980, 982, 984, 986, 988, 990, 992, 994, 996, 998, 1000, 1002, 1004, 1006, 1008, 1010, 1012, 1014, 1016, 1018, 1020, 1022, 1024, 1026, 1028, 1030, 1032, 1034, 1036, 1038, 1040, 1042, 1044, 1046, 1048, 1050, 1052, 1054, 1056, 1058, 1060, 1062, 1064, 1066, 1068, 1070, 1072, 1074, 1076, 1078, 1080, 1082, 1084, 1086, 1088, 1090, 1092, 1094, 1096, 1098, 1100, 1102, 1104, 1106, 1108, 1110, 1112, 1114, 1116, 1118, 1120, 1122, 1124, 1126, 1128, 1130, 1132, 1134, 1136, 1138, 1140, 1142, 1144, 1146, 1148, 1150, 1152, 1154, 1156, 1158, 1160, 1162, 1164, 1166, 1168, 1170, 1172, 1174, 1176, 1178, 1180, 1182, 1184, 1186, 1188, 1190, 1192, 1194, 1196, 1198, 1200, 1202, 1204, 1206, 1208, 1210, 1212, 1214, 1216, 1218, 1220, 1222, 1224, 1226, 1228, 1230, 1232, 1234, 1236, 1238, 1240, 1242, 1244, 1246, 1248, 1250, 1252, 1254, 1256, 1258, 1260, 1262, 1264, 1266, 1268, 1270, 1272, 1274, 1276, 1278, 1280, 1282, 1284, 1286, 1288, 1290, 1292, 1294, 1296, 1298, 1300, 1302, 1304, 1306, 1308, 1310, 1312, 1314, 1316, 1318, 1320, 1322, 1324, 1326, 1328, 1330, 1332, 1334, 1336, 1338, 1340, 1342, 1344, 1346, 1348, 1350, 1352, 1354, 1356, 1358, 1360, 1362, 1364, 1366, 1368, 1370, 1372, 1374, 1376, 1378, 1380, 1382, 1384, 1386, 1388, 1390, 1392, 1394, 1396, 1398, 1400, 1402, 1404, 1406, 1408, 1410, 1412, 1414, 1416, 1418, 1420, 1422, 1424, 1426, 1428, 1430, 1432, 1434, 1436, 1438, 1440, 1442, 1444, 1446, 1448, 1450, 1452, 1454, 1456, 1458, 1460, 1462, 1464, 1466, 1468, 1470, 1478, 1480, 1482, 1484, 1486, 1488, 1490, 1492, 1494, 1496, 1498, 1500, 1502, 1504, 1506, 1508, 1510, 1512, 1514, 1516, 1518, 1520, 1522, 1524, 1526, 1528, 1530, 1532, 1534, 1536, 1538, 1540, 1542, 1544, 1546, 1548, 1550, 1552, 1554, 1556, 1558, 1560, 1562, 1564, 1566, 1568, 1570, 1572, 1574, 1576, 1578, 1580, 1582, 1584, 1586, 1588, 1590, 1592, 1594, 1596, 1598, 1600, 1602, 1604, 1606, 1608, 1610, 1612, 1614, 1616, 1618, 1620, 1622, 1624, 1626, 1628, 1630, 1632, 1634, 1636, 1638, 1640, 1642, 1644, 1646, 1648, 1650, 1652, 1654, 1656, 1658, 1660, 1662, 1664, 1666, 1668, 1670, 1672, 1674, 1676, 1678, 1680, 1682, 1684, 1686, 1688, 1690, 1692, 1694, 1696, 1698, 1700, 1702, 1704, 1706, 1708, 1710, 1712, 1714, 1716, 1718, 1720, 1722, 1724, 1726, 1728, 1730, 1732, 1734, 1736, 1738, 1740, 1742, 1744, 1746, 1748, 1750, 1752, 1754, 1756, 1758, 1760, 1762, 1764, 1766, 1768, 1770, 1772, 1774, 1776, 1778, 1780, 1782, 1784, 1786, 1788, 1790, 1792, 1794, 1796, 1798, 1800, 1802, 1804, 1806, 1808, 1810, 1812, 1814, 1816, 1818, 1820, 1822, 1824, 1826, 1828, 1830, 1832, 1834, 1836, 1838, 1840, 1842, 1844, 1846, 1848, 1850, 1852, 1854, 1856, 1858, 1860, 1862, 1864, 1866, 1868, 1870, 1872, 1874, 1876, 1878, 1880, 1882, 1884, 1886, 1888, 1890, 1892, 1894, 1896, 1898, 1900, 1902, 1904, 1906, 1908, 1910, 1912, 1914, 1916, 1918, 1920, 1922, 1924, 1926, 1928, 1930, 1932, 1934, 1936, 1938, 1940, 1942, 1944, 1946, 1948, 1950, 1952, 1954, 1956, 1958, 1960, 1962, 1964, 1966, 1968, 1970, 1972, 1974, 1976, 1978, 1980, 1982, 1984, 1986, 1988, 1990, 1992, 1994, 1996, 1998, 2000, 2002. 2004, 2006, 2008, 2010, 2012, 2014, 2016, 2018, 2020, 2022, 2024, 2026, 2028, 2030, 2032, 2034, 2036, 2038, 2040, 2042, 2044, 2046, 2048, 2050, 2052, 2054, 2056, 2058, 2060, 2062, 2064, 2066, 2068, 2070, 2072, 2074, 2076, 2078, 2080, 2082, 2084, 2086, 2088, 2090, 2092, 2094, 2096, 2098, 2100, 2102, 2104, 2106, 2108, 2110, 2112, 2114, 2116, 2118, 2120, 2122, 2124, 2126, 2128, 2130, 2132, 2134, 2136, 2138, 2140, 2142, 2144, 2146, 2148, 2150, 2152, 2154, 2156, 2158, 2160, 2162, 2164, 2166, 2168, 2170, 2172, 2174, 2176, 2178, 2180, 2182, 2184, 2186, 2188, 2190, 2192, 2194, 2196, 2198, 2200, 2202, 2204, 2206, 2208, 2210, 2212, 2214, 2216, 2218, 2220, 2222, 2224, 2226, 2228, 2230, 2232, 2234, 2236, 2238, 2240, 2242, 2244, 2246, 2248, 2250, 2252, 2254, 2256, 2258, 2260, 2262, 2264, 2266, 2268, 2270, 2272, 2274, 2276, 2278, 2280, 2282, 2284, 2286, 2288, 2290, 2292, 2294, 2296, 2298, 2300, 2302, 2304, 2306, 2308, 2310, 2312, 2314, 2316, 2318, 2320, 2322, 2324, 2326, 2328, 2330, 2332, 2334, 2336, 2338, 2340, 2342, 2344, 2346, 2348, 2350, 2352, 2354, 2356, 2358, 2360, 2362, 2364, 2366, 2368, 2370, 2372, 2374, 2376, 2378, 2380, 2382, 2384, 2386, 2388, 2390, 2392, 2394, 2396, 2398, 2400, 2402, 2404, 2406, 2408, 2410, 2412, 2414, 2416, 2418, 2420, 2422, 2424, 2426, 2428, 2430, 2432, 2434, 2436, 2438, 2440, 2442, 2444, 2446, 2448, 2450, 2452, 2454, 2456, 2458, 2460, 2462, 2464, 2466, 2468, 2470, 2472, 2474, 2476, 2478, 2480, 2482, 2484, 2486, 2488, 2490, 2492, 2494, 2496, 2498, 2500, 2502, 2504, 2506, 2508, 2510, 2512, 2514, 2516, 2518, 2520, 2522, 2524, 2526, 2528, 2530, 2532, 2534, 2536, 2538, 2540, 2542, 2544, 2546, 2548, 2550, 2552, 2554, 2556, 2558, 2560, 2562, 2564, 2566, 2568, 2570, 2572, 2574, 2576, 2578, 2580, 2582, 2584, 2586, 2588, 2590, 2592, 2594, 2596, 2598, 2600, 2602, 2604, 2606, 2608, 2610, 2612, 2614, 2616, 2618, 2620, 2622, 2624, 2626, 2628, 2630, 2632, 2634, 2636, 2638, 2640, 2642, 2644, 2646, 2648, 2650, 2652, 2654, 2656, 2658, 2660, 2662, 2664, 2666, 2668, 2670, 2672, 2674, 2676, 2678, 2680, 2682, 2684, 2686, 2688, 2690, 2692, 2694, 2696, 2698, 2700, 2702, 2704, 2706, 2708, 2710, 2712, 2714, 2716, 2718, 2720, 2722, 2724, 2726, 2728, 2730, 2732, 2734, 2736, 2738, 2740, 2742, 2744, 2746, 2748, 2750, 2752, 2754, 2756, 2758, 2760, 2762, 2764, 2766, 2768, 2770, 2772, 2774, 2776, 2778, 2780, 2782, 2784, 2786, 2788, 2790, 2792, 2794, 2796, 2798, 2800, 2802, 2804, 2806, 28208, 2810, 2812, 2814, 2816, 2818, 2820, 2822, 2824, 2826, 2828, 2830, 2832, 2834, 2836, 2838, 2840, 2842, 2844, 2846, 2848, 2850, 2852, 2854, 2856, 2858, 2860, 2862, 2864, 2866, 2868, 2870, 2872, 2874, 2876, 2878, 2880, 2882, 2884, 2886, 2888, 2890, 2892, 2894, 2896, 2898, 2900, 2902, 2904, 2906, 2908, 2910, 2912, 2914, 2916, 2918, 2920, 2922, 2924, 2926, 2928, 2930, 2932, 2934, 2936, 2938, 2940, 2942, 2944, 2946, 2948, 2950, 2952, 2954, 2956, 2958, 2960, 2962, 2964, 2966, 2968, 2970, 2972, 2974, 2976, 2978, 2980, 2982, 2984, 2986, 2988, 2990, 2992, 2994, 2996, 2998, 3000, 3002, 3004, 3006, 3008, 3010, 3012, 3014, 3016, 3018, 3020, 3022, 3024, 3026, 3028, 3030, 3032, 3034, 3036, 3038, 3040, 3042, 3044, 3046, 3048, 3050, 3052, 3054, 3056, or 3058.

In some embodiments, the engineered RNA ligase comprises an amino acid sequence comprising residues 12 to 346 of an even numbered SEQ ID NO. of SEQ ID NOs: 4-1470 and 1478-3058, or an amino acid sequence comprising an even numbered SEQ ID NO. of SEQ ID NOs: 4-1470 and 1478-3058. In some embodiments, the amino acid sequence of the engineered RNA ligase optionally includes 1, 2, 3, 4, 5, 6, 7, 8, 9, or up to 10 substitutions, insertions, and/or deletions. In some embodiments, the amino acid sequence of the engineered RNA ligase optionally includes 1, 2, 3, 4, 5, 6, 7, 8, 9, or up to 10 substitutions. In some embodiments, the amino acid sequence of the engineered RNA ligase optionally includes 1, 2, 3, 4, or 5 substitutions, insertions, and/or deletions. In some embodiments, the amino acid sequence of the engineered RNA ligase optionally includes 1, 2, 3, 4, or 5 substitutions.

In some embodiments, the engineered RNA ligase comprises an amino acid sequence comprising residues 12 to 346 of SEQ ID NO: 4, 6, 8, 10, 12, 14, 16, 18, 20, 22, 24, 26, 28, 30, 32, 34, 336, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 322, 324, 326, 328, 330, 332, 334, 336, 338, 340, 342, 344, 346, 348, 350, 352, 354, 356, 358, 360, 362, 364, 366, 368, 370, 372, 374, 376, 378, 380, 382, 384, 386, 388, 390, 392, 394, 396, 398, 400, 402, 404, 406, 408, 410, 412, 414, 416, 418, 420, 422, 424, 426, 428, 430, 432, 434, 436, 438, 440, 442, 444, 446, 448, 450, 452, 454, 456, 458, 460, 462, 464, 466, 468, 470, 472, 474, 476, 478, 480, 482, 484, 486, 488, 490, 492, 494, 496, 498, 500, 502, 504, 506, 508, 510, 512, 514, 516, 518, 520, 522, 524, 526, 528, 530, 532, 534, 536, 538, 540, 542, 544, 546, 548, 550, 552, 554, 556, 558, 560, 562, 564, 566, 568, 570, 572, 574, 576, 578, 580, 582, 584, 586, 588, 590, 592, 594, 596, 598, 600, 602, 604, 606, 608, 610, 612, 614, 616, 618, 620, 622, 624, 626, 628, 630, 632, 634, 636, 638, 640, 642, 644, 646, 648, 650, 652, 654, 656, 658, 660, 662, 664, 666, 668, 670, 672, 674, 676, 678, 680, 682, 684, 686, 688, 690, 692, 694, 696, 698, 700, 702, 704, 706, 708, 710, 712, 714, 716, 718, 720, 722, 724, 726, 728, 730, 732, 734, 736, 738, 740, 742, 744, 746, 748, 750, 752, 754, 756, 758, 760, 762, 764, 766, 768, 770, 772, 774, 776, 778, 780, 782, 784, 786, 788, 790, 792, 794, 796, 798, 800, 802, 804, 806, 808, 810, 812, 814, 816, 818, 820, 822, 824, 826, 828, 830, 832, 834, 836, 838, 840, 842, 844, 846, 848, 850, 852, 854, 856, 858, 860, 862, 864, 866, 868, 870, 872, 874, 876, 878, 880, 882, 884, 886, 888, 890, 892, 894, 896, 898, 900, 902, 904, 906, 908, 910, 912, 914, 916, 918, 920, 922, 924, 926, 928, 930, 932, 934, 936, 938, 940, 942, 944, 946, 948, 950, 952, 954, 956, 958, 960, 962, 964, 966, 968, 970, 972, 974, 976, 978, 980, 982, 984, 986, 988, 990, 992, 994, 996, 998, 1000, 1002, 1004, 1006, 1008, 1010, 1012, 1014, 1016, 1018, 1020, 1022, 1024, 1026, 1028, 1030, 1032, 1034, 1036, 1038, 1040, 1042, 1044, 1046, 1048, 1050, 1052, 1054, 1056, 1058, 1060, 1062, 1064, 1066, 1068, 1070, 1072, 1074, 1076, 1078, 1080, 1082, 1084, 1086, 1088, 1090, 1092, 1094, 1096, 1098, 1100, 1102, 1104, 1106, 1108, 1110, 1112, 1114, 1116, 1118, 1120, 1122, 1124, 1126, 1128, 1130, 1132, 1134, 1136, 1138, 1140, 1142, 1144, 1146, 1148, 1150, 1152, 1154, 1156, 1158, 1160, 1162, 1164, 1166, 1168, 1170, 1172, 1174, 1176, 1178, 1180, 1182, 1184, 1186, 1188, 1190, 1192, 1194, 1196, 1198, 1200, 1202, 1204, 1206, 1208, 1210, 1212, 1214, 1216, 1218, 1220, 1222, 1224, 1226, 1228, 1230, 1232, 1234, 1236, 1238, 1240, 1242, 1244, 1246, 1248, 1250, 1252, 1254, 1256, 1258, 1260, 1262, 1264, 1266, 1268, 1270, 1272, 1274, 1276, 1278, 1280, 1282, 1284, 1286, 1288, 1290, 1292, 1294, 1296, 1298, 1300, 1302, 1304, 1306, 1308, 1310, 1312, 1314, 1316, 1318, 1320, 1322, 1324, 1326, 1328, 1330, 1332, 1334, 1336, 1338, 1340, 1342, 1344, 1346, 1348, 1350, 1352, 1354, 1356, 1358, 1360, 1362, 1364, 1366, 1368, 1370, 1372, 1374, 1376, 1378, 1380, 1382, 1384, 1386, 1388, 1390, 1392, 1394, 1396, 1398, 1400, 1402, 1404, 1406, 1408, 1410, 1412, 1414, 1416, 1418, 1420, 1422, 1424, 1426, 1428, 1430, 1432, 1434, 1436, 1438, 1440, 1442, 1444, 1446, 1448, 1450, 1452, 1454, 1456, 1458, 1460, 1462, 1464, 1466, 1468, 1470, 1478, 1480, 1482, 1484, 1486, 1488, 1490, 1492, 1494, 1496, 1498, 1500, 1502, 1504, 1506, 1508, 1510, 1512, 1514, 1516, 1518, 1520, 1522, 1524, 1526, 1528, 1530, 1532, 1534, 1536, 1538, 1540, 1542, 1544, 1546, 1548, 1550, 1552, 1554, 1556, 1558, 1560, 1562, 1564, 1566, 1568, 1570, 1572, 1574, 1576, 1578, 1580, 1582, 1584, 1586, 1588, 1590, 1592, 1594, 1596, 1598, 1600, 1602, 1604, 1606, 1608, 1610, 1612, 1614, 1616, 1618, 1620, 1622, 1624, 1626, 1628, 1630, 1632, 1634, 1636, 1638, 1640, 1642, 1644, 1646, 1648, 1650, 1652, 1654, 1656, 1658, 1660, 1662, 1664, 1666, 1668, 1670, 1672, 1674, 1676, 1678, 1680, 1682, 1684, 1686, 1688, 1690, 1692, 1694, 1696, 1698, 1700, 1702, 1704, 1706, 1708, 1710, 1712, 1714, 1716, 1718, 1720, 1722, 1724, 1726, 1728, 1730, 1732, 1734, 1736, 1738, 1740, 1742, 1744, 1746, 1748, 1750, 1752, 1754, 1756, 1758, 1760, 1762, 1764, 1766, 1768, 1770, 1772, 1774, 1776, 1778, 1780, 1782, 1784, 1786, 1788, 1790, 1792, 1794, 1796, 1798, 1800, 1802, 1804, 1806, 1808, 1810, 1812, 1814, 1816, 1818, 1820, 1822, 1824, 1826, 1828, 1830, 1832, 1834, 1836, 1838, 1840, 1842, 1844, 1846, 1848, 1850, 1852, 1854, 1856, 1858, 1860, 1862, 1864, 1866, 1868, 1870, 1872, 1874, 1876, 1878, 1880, 1882, 1884, 1886, 1888, 1890, 1892, 1894, 1896, 1898, 1900, 1902, 1904, 1906, 1908, 1910, 1912, 1914, 1916, 1918, 1920, 1922, 1924, 1926, 1928, 1930, 1932, 1934, 1936, 1938, 1940, 1942, 1944, 1946, 1948, 1950, 1952, 1954, 1956, 1958, 1960, 1962, 1964, 1966, 1968, 1970, 1972, 1974, 1976, 1978, 1980, 1982, 1984, 1986, 1988, 1990, 1992, 1994, 1996, 1998, 2000, 2002. 2004, 2006, 2008, 2010, 2012, 2014, 2016, 2018, 2020, 2022, 2024, 2026, 2028, 2030, 2032, 2034, 2036, 2038, 2040, 2042, 2044, 2046, 2048, 2050, 2052, 2054, 2056, 2058, 2060, 2062, 2064, 2066, 2068, 2070, 2072, 2074, 2076, 2078, 2080, 2082, 2084, 2086, 2088, 2090, 2092, 2094, 2096, 2098, 2100, 2102, 2104, 2106, 2108, 2110, 2112, 2114, 2116, 2118, 2120, 2122, 2124, 2126, 2128, 2130, 2132, 2134, 2136, 2138, 2140, 2142, 2144, 2146, 2148, 2150, 2152, 2154, 2156, 2158, 2160, 2162, 2164, 2166, 2168, 2170, 2172, 2174, 2176, 2178, 2180, 2182, 2184, 2186, 2188, 2190, 2192, 2194, 2196, 2198, 2200, 2202, 2204, 2206, 2208, 2210, 2212, 2214, 2216, 2218, 2220, 2222, 2224, 2226, 2228, 2230, 2232, 2234, 2236, 2238, 2240, 2242, 2244, 2246, 2248, 2250, 2252, 2254, 2256, 2258, 2260, 2262, 2264, 2266, 2268, 2270, 2272, 2274, 2276, 2278, 2280, 2282, 2284, 2286, 2288, 2290, 2292, 2294, 2296, 2298, 2300, 2302, 2304, 2306, 2308, 2310, 2312, 2314, 2316, 2318, 2320, 2322, 2324, 2326, 2328, 2330, 2332, 2334, 2336, 2338, 2340, 2342, 2344, 2346, 2348, 2350, 2352, 2354, 2356, 2358, 2360, 2362, 2364, 2366, 2368, 2370, 2372, 2374, 2376, 2378, 2380, 2382, 2384, 2386, 2388, 2390, 2392, 2394, 2396, 2398, 2400, 2402, 2404, 2406, 2408, 2410, 2412, 2414, 2416, 2418, 2420, 2422, 2424, 2426, 2428, 2430, 2432, 2434, 2436, 2438, 2440, 2442, 2444, 2446, 2448, 2450, 2452, 2454, 2456, 2458, 2460, 2462, 2464, 2466, 2468, 2470, 2472, 2474, 2476, 2478, 2480, 2482, 2484, 2486, 2488, 2490, 2492, 2494, 2496, 2498, 2500, 2502, 2504, 2506, 2508, 2510, 2512, 2514, 2516, 2518, 2520, 2522, 2524, 2526, 2528, 2530, 2532, 2534, 2536, 2538, 2540, 2542, 2544, 2546, 2548, 2550, 2552, 2554, 2556, 2558, 2560, 2562, 2564, 2566, 2568, 2570, 2572, 2574, 2576, 2578, 2580, 2582, 2584, 2586, 2588, 2590, 2592, 2594, 2596, 2598, 2600, 2602, 2604, 2606, 2608, 2610, 2612, 2614, 2616, 2618, 2620, 2622, 2624, 2626, 2628, 2630, 2632, 2634, 2636, 2638, 2640, 2642, 2644, 2646, 2648, 2650, 2652, 2654, 2656, 2658, 2660, 2662, 2664, 2666, 2668, 2670, 2672, 2674, 2676, 2678, 2680, 2682, 2684, 2686, 2688, 2690, 2692, 2694, 2696, 2698, 2700, 2702, 2704, 2706, 2708, 2710, 2712, 2714, 2716, 2718, 2720, 2722, 2724, 2726, 2728, 2730, 2732, 2734, 2736, 2738, 2740, 2742, 2744, 2746, 2748, 2750, 2752, 2754, 2756, 2758, 2760, 2762, 2764, 2766, 2768, 2770, 2772, 2774, 2776, 2778, 2780, 2782, 2784, 2786, 2788, 2790, 2792, 2794, 2796, 2798, 2800, 2802, 2804, 2806, 28208, 2810, 2812, 2814, 2816, 2818, 2820, 2822, 2824, 2826, 2828, 2830, 2832, 2834, 2836, 2838, 2840, 2842, 2844, 2846, 2848, 2850, 2852, 2854, 2856, 2858, 2860, 2862, 2864, 2866, 2868, 2870, 2872, 2874, 2876, 2878, 2880, 2882, 2884, 2886, 2888, 2890, 2892, 2894, 2896, 2898, 2900, 2902, 2904, 2906, 2908, 2910, 2912, 2914, 2916, 2918, 2920, 2922, 2924, 2926, 2928, 2930, 2932, 2934, 2936, 2938, 2940, 2942, 2944, 2946, 2948, 2950, 2952, 2954, 2956, 2958, 2960, 2962, 2964, 2966, 2968, 2970, 2972, 2974, 2976, 2978, 2980, 2982, 2984, 2986, 2988, 2990, 2992, 2994, 2996, 2998, 3000, 3002, 3004, 3006, 3008, 3010, 3012, 3014, 3016, 3018, 3020, 3022, 3024, 3026, 3028, 3030, 3032, 3034, 3036, 3038, 3040, 3042, 3044, 3046, 3048, 3050, 3052, 3054, 3056, or 3058. In some embodiments, the amino acid sequence of the engineered RNA ligase optionally includes 1, 2, 3, 4, 5, 6, 7, 8, 9, or up to 10 substitutions, insertions, and/or deletions. In some embodiments, the amino acid sequence of the engineered RNA ligase optionally includes 1, 2, 3, 4, 5, 6, 7, 8, 9, or up to 10 substitutions. In some embodiments, the amino acid sequence of the engineered RNA ligase optionally includes 1, 2, 3, 4, or 5 substitutions, insertions, and/or deletions. In some embodiments, the amino acid sequence of the engineered RNA ligase optionally includes 1, 2, 3, 4, or 5 substitutions.

In some embodiments, the engineered RNA ligase comprises an amino acid sequence comprising SEQ ID NO: 4, 6, 8, 10, 12, 14, 16, 18, 20, 22, 24, 26, 28, 30, 32, 34, 336, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 322, 324, 326, 328, 330, 332, 334, 336, 338, 340, 342, 344, 346, 348, 350, 352, 354, 356, 358, 360, 362, 364, 366, 368, 370, 372, 374, 376, 378, 380, 382, 384, 386, 388, 390, 392, 394, 396, 398, 400, 402, 404, 406, 408, 410, 412, 414, 416, 418, 420, 422, 424, 426, 428, 430, 432, 434, 436, 438, 440, 442, 444, 446, 448, 450, 452, 454, 456, 458, 460, 462, 464, 466, 468, 470, 472, 474, 476, 478, 480, 482, 484, 486, 488, 490, 492, 494, 496, 498, 500, 502, 504, 506, 508, 510, 512, 514, 516, 518, 520, 522, 524, 526, 528, 530, 532, 534, 536, 538, 540, 542, 544, 546, 548, 550, 552, 554, 556, 558, 560, 562, 564, 566, 568, 570, 572, 574, 576, 578, 580, 582, 584, 586, 588, 590, 592, 594, 596, 598, 600, 602, 604, 606, 608, 610, 612, 614, 616, 618, 620, 622, 624, 626, 628, 630, 632, 634, 636, 638, 640, 642, 644, 646, 648, 650, 652, 654, 656, 658, 660, 662, 664, 666, 668, 670, 672, 674, 676, 678, 680, 682, 684, 686, 688, 690, 692, 694, 696, 698, 700, 702, 704, 706, 708, 710, 712, 714, 716, 718, 720, 722, 724, 726, 728, 730, 732, 734, 736, 738, 740, 742, 744, 746, 748, 750, 752, 754, 756, 758, 760, 762, 764, 766, 768, 770, 772, 774, 776, 778, 780, 782, 784, 786, 788, 790, 792, 794, 796, 798, 800, 802, 804, 806, 808, 810, 812, 814, 816, 818, 820, 822, 824, 826, 828, 830, 832, 834, 836, 838, 840, 842, 844, 846, 848, 850, 852, 854, 856, 858, 860, 862, 864, 866, 868, 870, 872, 874, 876, 878, 880, 882, 884, 886, 888, 890, 892, 894, 896, 898, 900, 902, 904, 906, 908, 910, 912, 914, 916, 918, 920, 922, 924, 926, 928, 930, 932, 934, 936, 938, 940, 942, 944, 946, 948, 950, 952, 954, 956, 958, 960, 962, 964, 966, 968, 970, 972, 974, 976, 978, 980, 982, 984, 986, 988, 990, 992, 994, 996, 998, 1000, 1002, 1004, 1006, 1008, 1010, 1012, 1014, 1016, 1018, 1020, 1022, 1024, 1026, 1028, 1030, 1032, 1034, 1036, 1038, 1040, 1042, 1044, 1046, 1048, 1050, 1052, 1054, 1056, 1058, 1060, 1062, 1064, 1066, 1068, 1070, 1072, 1074, 1076, 1078, 1080, 1082, 1084, 1086, 1088, 1090, 1092, 1094, 1096, 1098, 1100, 1102, 1104, 1106, 1108, 1110, 1112, 1114, 1116, 1118, 1120, 1122, 1124, 1126, 1128, 1130, 1132, 1134, 1136, 1138, 1140, 1142, 1144, 1146, 1148, 1150, 1152, 1154, 1156, 1158, 1160, 1162, 1164, 1166, 1168, 1170, 1172, 1174, 1176, 1178, 1180, 1182, 1184, 1186, 1188, 1190, 1192, 1194, 1196, 1198, 1200, 1202, 1204, 1206, 1208, 1210, 1212, 1214, 1216, 1218, 1220, 1222, 1224, 1226, 1228, 1230, 1232, 1234, 1236, 1238, 1240, 1242, 1244, 1246, 1248, 1250, 1252, 1254, 1256, 1258, 1260, 1262, 1264, 1266, 1268, 1270, 1272, 1274, 1276, 1278, 1280, 1282, 1284, 1286, 1288, 1290, 1292, 1294, 1296, 1298, 1300, 1302, 1304, 1306, 1308, 1310, 1312, 1314, 1316, 1318, 1320, 1322, 1324, 1326, 1328, 1330, 1332, 1334, 1336, 1338, 1340, 1342, 1344, 1346, 1348, 1350, 1352, 1354, 1356, 1358, 1360, 1362, 1364, 1366, 1368, 1370, 1372, 1374, 1376, 1378, 1380, 1382, 1384, 1386, 1388, 1390, 1392, 1394, 1396, 1398, 1400, 1402, 1404, 1406, 1408, 1410, 1412, 1414, 1416, 1418, 1420, 1422, 1424, 1426, 1428, 1430, 1432, 1434, 1436, 1438, 1440, 1442, 1444, 1446, 1448, 1450, 1452, 1454, 1456, 1458, 1460, 1462, 1464, 1466, 1468, 1470, 1478, 1480, 1482, 1484, 1486, 1488, 1490, 1492, 1494, 1496, 1498, 1500, 1502, 1504, 1506, 1508, 1510, 1512, 1514, 1516, 1518, 1520, 1522, 1524, 1526, 1528, 1530, 1532, 1534, 1536, 1538, 1540, 1542, 1544, 1546, 1548, 1550, 1552, 1554, 1556, 1558, 1560, 1562, 1564, 1566, 1568, 1570, 1572, 1574, 1576, 1578, 1580, 1582, 1584, 1586, 1588, 1590, 1592, 1594, 1596, 1598, 1600, 1602, 1604, 1606, 1608, 1610, 1612, 1614, 1616, 1618, 1620, 1622, 1624, 1626, 1628, 1630, 1632, 1634, 1636, 1638, 1640, 1642, 1644, 1646, 1648, 1650, 1652, 1654, 1656, 1658, 1660, 1662, 1664, 1666, 1668, 1670, 1672, 1674, 1676, 1678, 1680, 1682, 1684, 1686, 1688, 1690, 1692, 1694, 1696, 1698, 1700, 1702, 1704, 1706, 1708, 1710, 1712, 1714, 1716, 1718, 1720, 1722, 1724, 1726, 1728, 1730, 1732, 1734, 1736, 1738, 1740, 1742, 1744, 1746, 1748, 1750, 1752, 1754, 1756, 1758, 1760, 1762, 1764, 1766, 1768, 1770, 1772, 1774, 1776, 1778, 1780, 1782, 1784, 1786, 1788, 1790, 1792, 1794, 1796, 1798, 1800, 1802, 1804, 1806, 1808, 1810, 1812, 1814, 1816, 1818, 1820, 1822, 1824, 1826, 1828, 1830, 1832, 1834, 1836, 1838, 1840, 1842, 1844, 1846, 1848, 1850, 1852, 1854, 1856, 1858, 1860, 1862, 1864, 1866, 1868, 1870, 1872, 1874, 1876, 1878, 1880, 1882, 1884, 1886, 1888, 1890, 1892, 1894, 1896, 1898, 1900, 1902, 1904, 1906, 1908, 1910, 1912, 1914, 1916, 1918, 1920, 1922, 1924, 1926, 1928, 1930, 1932, 1934, 1936, 1938, 1940, 1942, 1944, 1946, 1948, 1950, 1952, 1954, 1956, 1958, 1960, 1962, 1964, 1966, 1968, 1970, 1972, 1974, 1976, 1978, 1980, 1982, 1984, 1986, 1988, 1990, 1992, 1994, 1996, 1998, 2000, 2002. 2004, 2006, 2008, 2010, 2012, 2014, 2016, 2018, 2020, 2022, 2024, 2026, 2028, 2030, 2032, 2034, 2036, 2038, 2040, 2042, 2044, 2046, 2048, 2050, 2052, 2054, 2056, 2058, 2060, 2062, 2064, 2066, 2068, 2070, 2072, 2074, 2076, 2078, 2080, 2082, 2084, 2086, 2088, 2090, 2092, 2094, 2096, 2098, 2100, 2102, 2104, 2106, 2108, 2110, 2112, 2114, 2116, 2118, 2120, 2122, 2124, 2126, 2128, 2130, 2132, 2134, 2136, 2138, 2140, 2142, 2144, 2146, 2148, 2150, 2152, 2154, 2156, 2158, 2160, 2162, 2164, 2166, 2168, 2170, 2172, 2174, 2176, 2178, 2180, 2182, 2184, 2186, 2188, 2190, 2192, 2194, 2196, 2198, 2200, 2202, 2204, 2206, 2208, 2210, 2212, 2214, 2216, 2218, 2220, 2222, 2224, 2226, 2228, 2230, 2232, 2234, 2236, 2238, 2240, 2242, 2244, 2246, 2248, 2250, 2252, 2254, 2256, 2258, 2260, 2262, 2264, 2266, 2268, 2270, 2272, 2274, 2276, 2278, 2280, 2282, 2284, 2286, 2288, 2290, 2292, 2294, 2296, 2298, 2300, 2302, 2304, 2306, 2308, 2310, 2312, 2314, 2316, 2318, 2320, 2322, 2324, 2326, 2328, 2330, 2332, 2334, 2336, 2338, 2340, 2342, 2344, 2346, 2348, 2350, 2352, 2354, 2356, 2358, 2360, 2362, 2364, 2366, 2368, 2370, 2372, 2374, 2376, 2378, 2380, 2382, 2384, 2386, 2388, 2390, 2392, 2394, 2396, 2398, 2400, 2402, 2404, 2406, 2408, 2410, 2412, 2414, 2416, 2418, 2420, 2422, 2424, 2426, 2428, 2430, 2432, 2434, 2436, 2438, 2440, 2442, 2444, 2446, 2448, 2450, 2452, 2454, 2456, 2458, 2460, 2462, 2464, 2466, 2468, 2470, 2472, 2474, 2476, 2478, 2480, 2482, 2484, 2486, 2488, 2490, 2492, 2494, 2496, 2498, 2500, 2502, 2504, 2506, 2508, 2510, 2512, 2514, 2516, 2518, 2520, 2522, 2524, 2526, 2528, 2530, 2532, 2534, 2536, 2538, 2540, 2542, 2544, 2546, 2548, 2550, 2552, 2554, 2556, 2558, 2560, 2562, 2564, 2566, 2568, 2570, 2572, 2574, 2576, 2578, 2580, 2582, 2584, 2586, 2588, 2590, 2592, 2594, 2596, 2598, 2600, 2602, 2604, 2606, 2608, 2610, 2612, 2614, 2616, 2618, 2620, 2622, 2624, 2626, 2628, 2630, 2632, 2634, 2636, 2638, 2640, 2642, 2644, 2646, 2648, 2650, 2652, 2654, 2656, 2658, 2660, 2662, 2664, 2666, 2668, 2670, 2672, 2674, 2676, 2678, 2680, 2682, 2684, 2686, 2688, 2690, 2692, 2694, 2696, 2698, 2700, 2702, 2704, 2706, 2708, 2710, 2712, 2714, 2716, 2718, 2720, 2722, 2724, 2726, 2728, 2730, 2732, 2734, 2736, 2738, 2740, 2742, 2744, 2746, 2748, 2750, 2752, 2754, 2756, 2758, 2760, 2762, 2764, 2766, 2768, 2770, 2772, 2774, 2776, 2778, 2780, 2782, 2784, 2786, 2788, 2790, 2792, 2794, 2796, 2798, 2800, 2802, 2804, 2806, 28208, 2810, 2812, 2814, 2816, 2818, 2820, 2822, 2824, 2826, 2828, 2830, 2832, 2834, 2836, 2838, 2840, 2842, 2844, 2846, 2848, 2850, 2852, 2854, 2856, 2858, 2860, 2862, 2864, 2866, 2868, 2870, 2872, 2874, 2876, 2878, 2880, 2882, 2884, 2886, 2888, 2890, 2892, 2894, 2896, 2898, 2900, 2902, 2904, 2906, 2908, 2910, 2912, 2914, 2916, 2918, 2920, 2922, 2924, 2926, 2928, 2930, 2932, 2934, 2936, 2938, 2940, 2942, 2944, 2946, 2948, 2950, 2952, 2954, 2956, 2958, 2960, 2962, 2964, 2966, 2968, 2970, 2972, 2974, 2976, 2978, 2980, 2982, 2984, 2986, 2988, 2990, 2992, 2994, 2996, 2998, 3000, 3002, 3004, 3006, 3008, 3010, 3012, 3014, 3016, 3018, 3020, 3022, 3024, 3026, 3028, 3030, 3032, 3034, 3036, 3038, 3040, 3042, 3044, 3046, 3048, 3050, 3052, 3054, 3056, or 3058. In some embodiments, the amino acid sequence of the engineered RNA ligase optionally includes 1, 2, 3, 4, 5, 6, 7, 8, 9, or up to 10 substitutions, insertions, and/or deletions. In some embodiments, the amino acid sequence of the engineered RNA ligase optionally includes 1, 2, 3, 4, 5, 6, 7, 8, 9, or up to 10 substitutions. In some embodiments, the amino acid sequence of the engineered RNA ligase optionally includes 1, 2, 3, 4, or 5 substitutions, insertions, and/or deletions. In some embodiments, the amino acid sequence of the engineered RNA ligase optionally includes 1, 2, 3, 4, or 5 substitutions.

In some embodiments, the engineered RNA ligase comprises an amino acid sequence comprising residues 12 to 346 of SEQ ID NO: 54, 244, 648, 862, 956, 1088, 1416, 1612, 1840, 1866, 1954, 2062, 2674, 2754, or 2786, or an amino acid sequence comprising SEQ ID NO: 54, 244, 648, 862, 956, 1088, 1416, 1612, 1840, 1866, 1954, 2062, 2674, 2754, or 2786. In some embodiments, the amino acid sequence of the engineered RNA ligase optionally includes 1, 2, 3, 4, 5, 6, 7, 8, 9, or up to 10 substitutions, insertions, and/or deletions. In some embodiments, the amino acid sequence of the engineered RNA ligase optionally includes 1, 2, 3, 4, 5, 6, 7, 8, 9, or up to 10 substitutions. In some embodiments, the amino acid sequence of the engineered RNA ligase optionally includes 1, 2, 3, 4, or 5 substitutions, insertions, and/or deletions. In some embodiments, the amino acid sequence of the engineered RNA ligase optionally includes 1, 2, 3, 4, or 5 substitutions.

In some of the foregoing embodiments, the engineered RNA ligase polypeptide has 1, 2, 3, 4, or up to 5 substitutions in the amino acid sequence. In some embodiments, the engineered RNA ligase polypeptide has 1, 2, 3, or 4 substitutions in the amino acid sequence. In some embodiments, the substitutions comprises non-conservative or conservative substitutions. In some embodiments, the substitutions comprises conservative substitutions. In some embodiments, the substitutions comprises non-conservative substitutions. In some embodiments, guidance on non-conservative and conservative substitutions are provided by the variants disclosed herein.

In some embodiments, an engineered RNA ligase comprises an amino acid sequence having one or more amino acid differences in the N-terminal domain (also referred to herein as the adenyltransferase domain), the C-terminal domain, and/or the linker region connecting the N-terminal and C-terminal domains as compared to a reference RNA ligase. In some embodiments, the reference RNA ligase has an amino acid sequence corresponding to amino acid residues 12-346 of SEQ ID NO: 3060, which is the naturally occurring RNA ligase of Klebsiella phage KP179 (see, e.g., International patent publication WO2024138200, incorporated herein by reference). In some embodiments, the N-terminal domain corresponds to amino acid residues 12-246, the C-terminal domain corresponds to amino acid residues 256-346, and the linker region corresponds to amino acid residues 247-255 of the reference RNA ligase sequence of SEQ ID NO: 3060. The N-terminal, C-terminal, and linker region of other RNA ligases can be ascertained by sequence and/or structural alignments. By way of example and not limitation, for T4 RNA ligase 2, the N-terminal domain corresponds to amino acid residues 1-234, the C-terminal domain corresponds to amino acid residues 244-329, and the linker region corresponds to amino acid residues 235-243 of the amino acid sequence of naturally occurring T4 RNA ligase 2.

Without being bound by any theory of operation, the mechanism of a nucleic acid ligase involves the steps of (i) reaction of the ligase with ATP in the absence of nucleic acid to form a covalent ligase-AMP intermediate and corresponding release of pyrophosphate, (ii) binding of the ligase-AMP intermediate to a nucleic acid containing a nick, and transfer of the adenylate to the 5′-phosphate of the donor strand to form a adenylated nicked intermediate, and (iii) a ligase mediated attack of the 3′-OH of the acceptor strand on the 5′-phosphoanhydride linkage, resulting in a 3′-5′ phosphodiester linkage and release of AMP.

As used herein, the N-terminal domain, also referred to as the adenyltransferase domain, refers to the region or domain that mediates reaction of the ligase with ATP to form a covalent ligase-AMP intermediate with the release of pyrophosphate, and attack of the 3′-OH of the nick on the 5′-phosphoanhydride linkage, resulting in the closure or repaired 3′-5′ phosphodiester and release of AMP. This corresponds to catalytic steps (i) and (iii) above. The C-terminal domain refers to a region or domain of the ligase that mediates catalytic step (ii). The linker region connecting the N-terminal and the C-terminal domains acts as a fulcrum for domain movements that accompany substrate binding and reaction chemistry (see, e.g., Nandakumar et al., Cell, 2006, 127 (1): 71-84).

In some embodiments, the amino acid sequence of the engineered RNA ligase comprises
(a) one or more amino acid differences in the N-terminal domain at amino acid positions 12, 13, 15, 17, 18, 21, 23, 25, 26, 28, 29, 30, 32, 33, 37, 38, 39, 40, 41, 42, 45, 52, 54, 56, 57, 58, 60, 61, 62, 64, 65, 67, 71, 73, 74, 75, 77, 80, 81, 83, 84, 86, 87, 89, 90, 91, 93, 95, 97, 99, 100, 101, 102, 103, 104, 105, 106, 107, 109, 113, 114, 116, 117, 119, 121, 122, 123, 125, 126, 127, 128, 129, 131, 135, 137, 140, 141, 143, 144, 148, 150, 154, 156, 157, 158, 159, 160, 161, 162, 165, 169, 170, 171, 173, 175, 177, 178, 179, 181, 183, 184, 185, 190, 191, 192, 193, 194, 197, 198, 204, 206, 208, 210, 212, 213, 214, 215, 218, 223, 224, 226, 228, 229, 230, 232, 236, 238, 241, and 243, or at equivalent positions thereof;
(b) one or more amino acid differences in the C-terminal domain at amino acid positions 256, 257, 258, 260, 261, 264, 265, 266, 267, 268, 270, 271, 272, 274, 276, 277, 280, 282, 283, 286, 288, 289, 290, 291, 292, 294, 295, 296, 297, 298, 299, 300, 301, 302, 305, 306, 307, 310, 311, 315, 316, 317, 318, 323, 324, 325, 326, 327, 330, 331, 332, 334, 335, 336, 337, 338, 341, 342, 344, 345, and 346, or at equivalent positions thereof; and/or
(c) one or more amino acid differences in the linker region connecting the N-terminal and C-terminal domains at one or more amino acid positions 247, 248, 249, 250, 251, 252, 253, 254, and 255, or at equivalent positions thereof,
wherein the amino acid positions are relative to the reference sequence corresponding to residues 12-346 of SEQ ID NO: 3060.

In some embodiments, the amino acid sequence of the engineered RNA ligase comprises
(a) in the N-terminal domain one or more amino acid residues 12A/F/L/M/P/R/T/V, 13W, 15M, 17P, 18A/H, 21S, 23K/T, 25A/D/F/G/L/M/S/T, 26L, 28A/L/M/V, 29D/S, 30A/L/T/V/W, 32E, 33A/I/L/T/V, 37A/F/K/L/M/R, 38A/E/L/R/S/T/V, 39P, 40D/I/P/S, 41E/I, 42T, 45S, 52C, 54L, 56I/L, 57V, 58M, 60A/C/D/R/S/V, 61A, 62A/V, 64A/Q/W, 65C, 67R, 71T, 73Q/W, 74D, 75A/D, 77M, 80E, 81Y, 83A/E/G/L/T/V, 84V, 86D/G/L, 87E, 89C/D/E/G/L/M/N/Q/R/S/T/V/W/Y, 90E/F, 91A/E/V, 93K/V, 95A/C/G/I, 97A/E, 991, 100A/G/H/L/R/S/T/V, 101P/V/W, 102T, 103G/V, 104T, 105K/L/R, 106G/L/M/Q/R/V/W, 107M/S, 109F/L, 113L/M, 114E, 116L, 117A/Q, 119L, 121N, 122C/F/G/L/Q/T/W, 123K/R, 125K/T, 126M, 127P, 128E, 129L/W, 131T, 135A/H/M/Q/V, 137N/R, 140I, 141D/G, 143A/C/E/F/H/M/S/T/V, 144L, 148F, 150D/L, 154S/V, 156G/H/Q/S, 157F/L/Q/V, 158R/S, 159F, 160R, 161M/V, 162C, 165M, 169G/N, 170H/Y, 171S, 173M, 175A, 177K, 178V, 179E, 181E/N/P/R/S, 183L/V, 184L, 185C/F/G/K/M/P/S/T/V, 190I/M, 191C, 192D/E/F/L/N, 193K/Q, 194A/G/V, 197W, 198M, 204W, 206L, 208H/I/K/L/P/T, 210G/V, 212E/G/I/K/L, 213D/N/R/T, 214A/C/H/K/L/Q/Y, 215G/T, 218F, 223C, 224A/Q/R, 226R, 228D/L, 229W, 230T/V, 232G/M/N/R/S, 236L/V, 238K/S/V, 241E/G/Q/R, and 243H, or at equivalent positions thereof;
(b) in the C-terminal domain one or more amino acid residues 256A/E/G/M/S/W, 257D/E/G, 258D/M/P/V, 260G, 261D/P/T, 264A/E/G/S/V, 265A/F, 266C/I/L, 267I/M, 268F/G/M, 270L/M/V, 271A/E/G/T, 272M/S, 274A, 276A/G/M/V, 277A, 280L/N/W, 282A/M, 283C/V, 286D/E/F/M/V, 288E/L/R/W, 289A/D/E/I/P/T, 290D/H/N/P, 291H/L/Q/R/S/T/W, 292N/S, 294A/S/V/Y, 295C/F/G/K/L/M/P/Q/R/S/T/V/Y,296E/G/L/Q/W, 297A/I/L, 298C/I/K/R/S/V, 299E/I/K/Q/R/T/V, 300A, 301A/F/I/L/M/V, 302A/C/I/L/S, 305V, 306G/L/T, 307K/R, 310H/L/M/N/Q/R/S/T, 311C, 315E/S, 316I/L/Y, 317E/F/G/Q/V/W, 318A/E/G/S, 323E/G, 324D/G/M, 325L, 326C/L, 327E/G/M/S, 330A/D/E/G/K/R, 331D/G/R/S/W, 332E/H/W, 334E/R, 335H/Q, 336I/L/M, 337A/V, 338G/L/M, 341A, 342R/S/T, 344K, 345Q/S, and 346S; or at equivalent positions thereof; and/or
(c) in the linker region connecting the N-terminal and C-terminal domains one or more amino acid residues 247I/L/R/S, 248F/G/I/K/T, 249K/L/R/S, 250E/I/S, 251C/E/F/G/L/R/S/T/Y, 252A/Q, 253L/W, 254E/K/L/N/Q/S/V, and 255N/S/V/Y, or at equivalent positions thereof,
wherein the amino acid positions are relative to the reference sequence corresponding to residues 12-346 of SEQ ID NO: 3060.

In some embodiments, the N-terminal domain of the engineered RNA ligase comprises one or more amino acid residues 12A/F/L/P/R/T/V, 23K/T, 25A/D/F/G/L/M/S/T, 29D/S, 37A/F/K/L/M/R, 38A/E/L/R/S/T/V, 75A/D, 86D/L, 89C/D/E/L/N/Q/R/S/T/V/Y, 93K, 101P/V/W, 122C/F/G/L/Q/T/W, 143A/C/E/F/H/M/T/V, 157F/Q/V, 183L/V, 192E/F/L/N, 194A/G/V, 198M, 212E/G/I/K/L, 214A/C/H/K/L/Q/Y, 230T/V, and 241E/G/Q/R. or at equivalent positions thereof, wherein the amino acid positions are relative to the reference sequence corresponding to residues 12-346 of SEQ ID NO: 3060.

In some embodiments, the N-terminal domain of the engineered RNA ligase comprises one or more amino acid residues 12T, 23T, 25A, 29S, 37L, 38A/S, 75D, 86D, 89M, 93K, 101P, 122F, 157V, 183L, 192E, 194V, 198M, 212E, 214L, 226R, 230T/V, and 241G, or at equivalent positions thereof, wherein the amino acid positions are relative to the reference sequence corresponding to residues 12-346 of SEQ ID NO: 3060.

In some embodiments, the C-terminal domain of the engineered RNA ligase comprises one or more amino acid residues 266C/I/L, 268F/G/M, 280L/N/W, 291H/L/Q/R/S/T/W, 295C/F/K/L/M/P/Q/R/S/T/V/Y, 298C/I/K/R/S/V, 307K/R, 316I/L/Y, 318A/E/G/S, 326C/L, 330A/D/E/G/K/R, and 331D/G/R/S/W, or at equivalent positions thereof, wherein the amino acid positions are relative to the reference sequence corresponding to residues 12-346 of SEQ ID NO: 3060.

In some embodiments, the C-terminal domain of the engineered RNA ligase comprises one or more amino acid residues 266L, 268G, 280L, 291Q, 295V, 298S/V, 307R, 316L, 318S, 326L, 330A, and 331G, or at equivalent positions thereof, wherein the amino acid positions are relative to the reference sequence corresponding to residues 12-346 of SEQ ID NO: 3060.

In some embodiments, the linker region connecting the N-terminal and C-terminal domains comprises one or more amino acid residues 248G/I/K/T, 249K/L/R/S, 250E/I/S, 251C/E/F/G/L/R/S/T/Y, and 254E/K/L/N/Q/S/V, or at equivalent positions thereof, wherein the amino acid positions are relative to the reference sequence corresponding to residues 12-346 of SEQ ID NO: 3060.

In some embodiments, the linker region connecting the N-terminal and C-terminal domains comprises one or more amino acid residues 249L, 2501, 251E/S, and 254S, or at equivalent positions thereof, wherein the amino acid positions are relative to the reference sequence corresponding to residues 12-346 of SEQ ID NO: 3060.

In some embodiments, the engineered RNA ligase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99% or more sequence identity to the N-terminal domain corresponding to amino acid residues 12-246 of SEQ ID NO: 3060, wherein the amino acid sequence of the RNA ligase comprises one or more amino acid differences or amino acid residues in the N-terminal domain, as provided in the foregoing.

In some embodiments, the engineered RNA ligase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99% or more sequence identity to the C-terminal domain corresponding to amino acid residues 256-346 of SEQ ID NO: 3060, wherein the amino acid sequence of the RNA ligase comprises one or more amino acid differences or amino acid residues in the C-terminal domain, as provided in the foregoing.

In some embodiments, the engineered RNA ligase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99% or more sequence identity to the linker region corresponding to amino acid residues 247-255 of the reference sequence of SEQ ID NO: 3060, wherein the amino acid sequence of the RNA ligase comprises one or more amino acid differences or amino acid residues in the linker region, as provided in the foregoing.

In some embodiments, the engineered RNA ligase of the present disclosure has RNA ligase 2 activity, particularly with the improved or enhanced properties described herein. In some embodiments, the engineered RNA ligase has at least one improved or enhanced property as compared to a reference RNA ligase. Exemplary improved properties are provided in the Examples.

In some embodiments, the engineered RNA ligase has increased activity as compared to the reference RNA ligase. In some embodiments, the engineered RNA ligase has at least 1.1 fold, 1.2 fold, 1.3 fold, 1.4 fold, 1.5 fold, 2-fold, 3-fold, 4-fold, 5-fold, 10-fold, 15-fold, 20-fold, or more fold activity compared to the reference RNA ligase. In some embodiments, the increased activity is on modified polynucleotide substrates, as discussed herein and in the Examples.

In some embodiments, the engineered RNA ligase exhibits increased product yield as compared to the reference RNA ligase. In some embodiments, the engineered RNA ligase exhibits increased product yield at substrate concentration of 10 uM to 10 uM, 20 uM to 350 uM, 50 uM to 300 uM, or 100 uM to 250 uM, preferably 200 to 400 uM as compared to the reference RNA ligase. In some embodiments, the engineered RNA ligase exhibits increased product yield at substrate concentration of 10 uM, 20 uM, 50 uM, 100 uM, 150 uM, 200 uM, 250 uM, 300 uM, 350 uM, or 400 uM or higher as compared to the reference RNA ligase. In some embodiments, the engineered RNA ligase exhibits increased product yield at a substrate concentration of 1-10 mM, 2-8 mM, or 4-6 mM. In some embodiments, the engineered RNA ligase exhibits increased product yield at 1 mM, 2 mM, 3 mM, 4 mM, 5 mM, 6 mM, 7 mM, 8 mM, 9 mM, or about 10 mM.

In some embodiments, the engineered RNA ligase has increased activity on polynucleotides substrates with phosphorothioate internucleoside linkages as compared to the reference RNA ligase.

In some embodiments, the engineered RNA ligase has increased activity or product yield on polynucleotides substrates with 2′-modifications (e.g., 2′-O-methyl and/or 2′-fluoro) as compared to the reference RNA ligase. In particular, the engineered RNA ligase has increased activity on polynucleotide substrates having a 2′-modified sugar moiety on the 3′-terminal nucleotide and/or 5′-terminal nucleotide, where the 3′-terminal nucleotide on the polynucleotide acceptor strand is being ligated to the 5′-terminal nucleotide of the polynucleotide donor strand substrates.

In some embodiments, the engineered RNA ligase exhibits for double stranded ligase substrates a efficiency of at least 90%, 91%, 92%, 93%, 94%, 95%, 96%, 97%, 98%, 99%, or greater on polynucleotide substrates comprising a modified nucleoside at the 3′-terminal nucleoside of the polynucleotide acceptor strand. In some embodiments, the engineered RNA ligase exhibits a ligation efficiency of at least 90%, 91%, 92%, 93%, 94%, 95%, 96%, 97%, 98%, 99%, or greater for a polynucleotide acceptor strand having a modified 3′-terminal nucleoside of 2′-fluoro-adenosine, 2′-fluoro-guanosine, 2′-fluoro-cytidine, 2′-fluoro-uridine, 2′-fluoro-thymidine, 2′-O-methyl-adenosine, 2′-O-methyl-guanosine, 2′-O-methyl-cytidine, 2′-O-methyl-uridine, or 2′-O-methyl-thymidine.

In some embodiments, In some embodiments, the engineered RNA ligase exhibits a ligation efficiency of at least 90%, 91%, 92%, 93%, 94%, 95%, 96%, 97%, 98%, 99%, or greater for a polynucleotide donor strand having a modified 5′-terminal nucleoside of 2′-fluoro-adenosine, 2′-fluoro-guanosine, 2′-fluoro-cytidine, 2′-fluoro-uridine, 2′-fluoro-thymidine, 2′-O-methyl-adenosine, 2′-O-methyl-guanosine, 2′-O-methyl-cytidine, 2′-O-methyl-uridine, or 2′-O-methyl-thymidine.

In some embodiments, the engineered RNA ligase exhibits for double stranded ligase substrates a efficiency of at least 90%, 91%, 92%, 93%, 94%, 95%, 96%, 97%, 98%, 99%, or greater on polynucleotide substrates comprising a modified nucleoside at the 3′-terminal nucleoside of the polynucleotide acceptor strand. In some embodiments, the engineered RNA ligase exhibits a ligation efficiency of at least 90%, 91%, 92%, 93%, 94%, 95%, 96%, 97%, 98%, 99%, or greater for ligation of a polynucleotide acceptor strand having a modified 3′-terminal nucleoside of 2′-fluoro-adenosine, 2′-fluoro-guanosine, 2′-fluoro-cytidine, 2′-fluoro-uridine, 2′-fluoro-thymidine, 2′-O-methyl-adenosine, 2′-O-methyl-guanosine, 2′-O-methyl-cytidine, 2′-O-methyl-uridine, or 2′-O-methyl-thymidine, and a polynucleotide donor strand having a modified 5′-terminal nucleoside of 2′-fluoro-adenosine, 2′-fluoro-guanosine, 2′-fluoro-cytidine, 2′-fluoro-uridine, 2′-fluoro-thymidine, 2′-O-methyl-adenosine, 2′-O-methyl-guanosine, 2′-O-methyl-cytidine, 2′-O-methyl-uridine, or 2′-O-methyl-thymidine.

In some embodiments, the engineered RNA ligase exhibits for double stranded ligase substrates a efficiency of at least 90%, 91%, 92%, 93%, 94%, 95%, 96%, 97%, 98%, 99%, or greater on polynucleotide substrates comprising a modified nucleoside at the 3′-terminal nucleoside of the polynucleotide acceptor strand. In some embodiments, the engineered RNA ligase exhibits a ligation efficiency of at least 90%, 91%, 92%, 93%, 94%, 95%, 96%, 97%, 98%, 99%, or greater for ligation of a ligase substrate comprising:
a polynucleotide acceptor strand having a modified 3′-terminal nucleoside of 2′-fluoro-adenosine, 2′-fluoro-guanosine, 2′-fluoro-cytidine, 2′-fluoro-uridine, 2′-fluoro-thymidine, 2′-O-methyl-adenosine, 2′-O-methyl-guanosine, 2′-O-methyl-cytidine, 2′-O-methyl-uridine, or 2′-fluoro-thymidine;
a polynucleotide donor strand having a modified 5′-terminal nucleoside of 2′-fluoro-adenosine, 2′-fluoro-guanosine, 2′-fluoro-cytidine, 2′-fluoro-uridine, 2′-fluoro-thymidine, 2′-O-methyl-adenosine, 2′-O-methyl-guanosine, 2′-O-methyl-cytidine, 2′-O-methyl-uridine, or 2′-fluoro-thymidine; and
a polynucleotide complementary to the polynucleotide acceptor strand and polynucleotide donor strand, wherein the polynucleotide complementary to the polynucleotide acceptor strand and polynucleotide donor strand comprises a complementary 2′-fluoro or 2′-O-methyl nucleoside at the position complementary to the 3′-terminal nucleoside of the polynucleotide acceptor strand, and/or a complementary 2-fluoro or 2-O-methyl nucleoside at the position complementary to the 5′-terminal nucleoside of the polynucleotide donor strand.

In some embodiments, the reference RNA ligase has the sequence corresponding to residues 12 to 346 of SEQ ID NO: 2, 54, 244, 648, 862, 956, 1088, 1416, 1612, 1840, 1866, 1954, 2062, 2674, 2754, or 2786, or the sequence corresponding to SEQ ID NO: 2, 54, 244, 648, 862, 956, 1088, 1416, 1612, 1840, 1866, 1954, 2062, 2674, 2754, or 2786. In some embodiments, the reference RNA ligase has the sequence corresponding to residues 12 to 346 of SEQ ID NO: 2, or the sequence corresponding to SEQ ID NO: 2.

In some embodiments, the engineered RNA ligase has one or more improved property selected from i) increased activity, ii) increased stability, iii) increased thermostability, iv) increased product yield, v) increased activity on polynucleotides ligase substrates with phosphorothioate internucleoside linkages, vi) increased activity on oligonucleotides with 2′-modifications, vii) increased substrate tolerance, or any combination of i), ii), iii), iv), v), vi), and vii), compared to a reference RNA ligase. In some embodiments, the improved property of the engineered RNA ligase is in comparison to the reference RNA ligase having the sequence corresponding to residues 12 to 346 of SEQ ID NO: 2, 54, 244, 648, 862, 956, 1088, 1416, 1612, 1840, 1866, 1954, 2062, 2674, 2754, or 2786, or the sequence corresponding to SEQ ID NO: 2, 54, 244, 648, 862, 956, 1088, 1416, 1612, 1840, 1866, 1954, 2062, 2674, 2754, or 2786. In some embodiments, the reference RNA ligase has the sequence corresponding to residues 12 to 346 of SEQ ID NO: 2, or the sequence corresponding to SEQ ID NO: 2.

In some embodiments, the engineered RNA ligase is expressed as a fusion protein. In some embodiments, the engineered RNA ligase described herein can be fused to a variety of polypeptide sequences, such as, by way of example and not limitation, polypeptide tags that can be used for detection and/or purification, and nucleic binding domains to enhance activity. In some embodiments, the fusion protein of the engineered RNA ligase comprises a glycine-histidine or histidine-tag (His-tag). In some embodiments, the fusion protein of the engineered RNA ligase comprises a polylysine, for example, 2-12 contiguous lysine residues. In some embodiments, the fusion protein of the engineered RNA ligase comprises an epitope tag, such as c-myc, FLAG, V5, or hemagglutinin (HA). In some embodiments, the fusion protein of the engineered RNA ligase comprises a GST, SUMO, Strep, MBP, or GFP tag. In some embodiments, the engineered RNA ligase is fused to protein domains that bind nucleic acids, particularly RNA, for example, zinc knuckle (zk), helix zinc knuckle (hzk), RGG/RG box motifs, RNase III (rnc), and homeodomains (see, e.g., Tong et al., Nucleic Acids Res., 50 (19): 11175-11185, incorporated by reference herein). In some embodiments, the fusion is to the amino (N—) terminus of engineered RNA ligase polypeptide. In some embodiments, the fusion is to the carboxy (C—) terminus of the engineered RNA ligase polypeptide.

In some embodiments, the engineered RNA ligase polypeptide described herein is an isolated composition. In some embodiments, the engineered RNA ligase polypeptide is purified. In some embodiments, the engineered RNA ligase is provided in solution, as a lyophilizate, or immobilized on a substrate, as further discussed herein.

In some embodiments, the present disclosure further provides functional fragments or biologically active fragments of engineered RNA ligase polypeptides described herein. Thus, for each and every embodiment herein of an engineered RNA ligase, a functional fragment or biologically active fragment of the engineered RNA ligase is provided herewith. In some embodiments, a functional fragment or biologically active fragments of an engineered RNA ligase comprises at least about 90%, 91%, 92%, 93%, 94%, 95%, 96%, 97%, 98%, or 99% of the activity of the RNA ligase polypeptide from which it was derived (i.e., the parent RNA ligase). In some embodiments, functional fragments or biologically active fragments comprise at least 90%, 91%, 92%, 93%, 94%, 95%, 96%, 97%, 98%, or 99% of the parent sequence of the RNA ligase. In some embodiments, the functional fragment will be truncated by less than 5, less than 10, less than 15, less than 10, less than 25, less than 30, less than 35, less than 40, less than 45, less than 50 amino acids, less than 55 amino acids, less than 60 amino acids, less than 65 amino acids, or less than 70 amino acids.

In some embodiments, a functional fragment of an engineered RNA ligase herein comprises at least 70%, 75%, 80%, 81%, 82%, 83%, 84%, 85%, 90%, 91%, 92%, 93%, 94%, 95%, 96%, 97%, 98%, or 99% of the parent sequence of the engineered RNA ligase. In some embodiments, the functional fragment will be truncated by less than 5, less than 10, less than 15, less than 10, less than 25, less than 30, less than 35, less than 40, less than 45, less than 50, less than 55, less than 60, less than 65, or less than 70 amino acids.

In some embodiments, the functional fragments or biologically active fragments of the engineered RNA ligase polypeptide described herein include at least a mutation or mutation set in the amino acid sequence of the engineered RNA ligase described herein. Accordingly, in some embodiments, the functional fragments or biologically active fragments of the engineered RNA ligase displays the enhanced or improved property associated with the mutation or mutation set in the parent RNA ligase.

### Polynucleotides Encoding Engineered Polypeptides, Expression Vectors and Host Cells

In another aspect, the present disclosure provides recombinant polynucleotides encoding the engineered RNA ligase described herein. In some embodiments, the recombinant polynucleotides are operably linked to one or more heterologous regulatory sequences that control gene expression to create a recombinant polynucleotide construct capable of expressing the engineered RNA ligase.

As will be apparent to the skilled artisan, availability of a protein sequence and the knowledge of the codons corresponding to the various amino acids provide a description of all the polynucleotides capable of encoding the subject polypeptides. The degeneracy of the genetic code, where the same amino acids are encoded by alternative or synonymous codons, allows an extremely large number of nucleic acids to be made, all of which encode an engineered RNA ligase of the present disclosure. Thus, the present disclosure provides methods and compositions for the production of each and every possible variation of polynucleotides that could be made that encode the engineered RNA ligase polypeptides described herein by selecting combinations based on the possible codon choices, and all such variations of polynucleotides are to be considered specifically disclosed for any polypeptide described herein, including the amino acid sequences presented in the Examples (e.g., in Tables 5.1, 6.1, 6.2, 7.1, 8.1, 8.2, 9.1, 10.1, 11.1, 12.1, 13.1, 14.1, 15.1, 16.1, 17.1,18.1, 19.1, 20.1, 21.1, 22.1, 23.1, 24.1, 25.1, and 26.1,) and in the accompanying Sequence Listing.

In some embodiments, the codons are preferably optimized for utilization by the chosen host cell for protein production. In some embodiments, preferred codons in bacterial cells are used for expression in bacterial cells. In some embodiments, preferred codons in fungal cells are used for expression in fungal cells. In some embodiments, preferred codons in insect cells are used for expression in insect cells. In some embodiments, preferred codons in mammalian cells are used for expression in mammalian cells. In some embodiments, codon optimized polynucleotides encoding an engineered RNA ligase polypeptide described herein contain preferred codons at about 40%, 50%, 60%, 70%, 80%, 90%, or greater than 90% of the codon positions in the full length coding region.

Accordingly, in some embodiments, a recombinant polynucleotide of the present disclosure encodes an engineered RNA ligase polypeptides described herein. In some embodiments, the polynucleotide sequence of the recombinant polynucleotide is codon optimized.

In some embodiments, the recombinant polynucleotide comprises a polynucleotide sequence encoding an engineered RNA ligase comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to a reference sequence corresponding to residues 12 to 346 of an even-numbered SEQ ID NO. of SEQ ID NOs: 2-1470 and 1478-3058, or to a reference sequence corresponding to an even-numbered SEQ ID NO. of SEQ ID NOs: 2-1470 and 1478-3058, wherein the amino acid sequence comprises one or more substitutions relative to the reference sequence corresponding to residues 12 to 346 of SEQ ID NO: 2, 54, 244, 648, 862, 956, 1088, 1416, 1612, 1840, 1866, 1954, 2062, 2674, 2754, or 2786, or relative to the reference sequence corresponding to SEQ ID NO: 2, 54, 244, 648, 862, 956, 1088, 1416, 1612, 1840, 1866, 1954, 2062, 2674, 2754, or 2786.

In some embodiments, the recombinant polynucleotide comprises a polynucleotide sequence encoding an engineered RNA ligase comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12 to 346 of SEQ ID NO: 2, 54, 244, 648, 862, 956, 1088, 1416, 1612, 1840, 1866, 1954, 2062, 2674, 2754, or 2786, or to the reference sequence corresponding to SEQ ID NO: 2, 54, 244, 648, 862, 956, 1088, 1416, 1612, 1840, 1866, 1954, 2062, 2674, 2754, or 2786, wherein the amino acid sequence comprises one or more substitutions relative to the reference sequence corresponding to residues 12 to 346 of SEQ ID NO: 2, 54, 244, 648, 862, 956, 1088, 1416, 1612, 1840, 1866, 1954, 2062, 2674, 2754, or 2786, or relative to the reference sequence corresponding to SEQ ID NO: 2, 54, 244, 648, 862, 956, 1088, 1416, 1612, 1840, 1866, 1954, 2062, 2674, 2754, or 2786.

In some embodiments, the recombinant polynucleotide comprises a polynucleotide sequence encoding an engineered RNA ligase comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12 to 346 of SEQ ID NO: 2, or to the reference sequence corresponding to SEQ ID NO: 2, wherein the amino acid sequence comprises one or more substitutions relative to the reference sequence corresponding to residues 12 to 346 of SEQ ID NO: 2, or relative to the reference sequence corresponding to SEQ ID NO: 2.

In some embodiments, the recombinant polynucleotide comprises a polynucleotide sequence encoding an engineered RNA ligase comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12 to 346 of SEQ ID NO: 54, 244, 648, 862, 956, 1088, 1416, 1612, 1840, 1866, 1954, 2062, 2674, 2754, or 2786, or to the reference sequence corresponding to SEQ ID NO: 54, 244, 648, 862, 956, 1088, 1416, 1612, 1840, 1866, 1954, 2062, 2674, 2754, or 2786, wherein the amino acid sequence comprises one or more substitutions relative to the reference sequence corresponding to residues 12 to 346 of SEQ ID NO: 2, or relative to the reference sequence corresponding to SEQ ID NO: 2.

In some embodiments, the recombinant polynucleotide comprises a polynucleotide sequence encoding an engineered RNA ligase comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12 to 346 of an even-numbered SEQ ID NO. of SEQ ID NOs: 4-1470 and 1478-3058, or to the reference sequence corresponding to an even-numbered SEQ ID NO. of SEQ ID NOs: 4-1470 and 1478-3058, wherein the amino acid sequence comprises one or more substitutions relative to the reference sequence corresponding to residues 12 to 346 of SEQ ID NO: 2, or relative to the reference sequence corresponding to SEQ ID NO: 2.

In some embodiments, the recombinant polynucleotide comprises a polynucleotide sequence encoding an engineered RNA ligase comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12 to 346 of an even-numbered SEQ ID NO. of SEQ ID NOs: 4-218, or to the reference sequence corresponding to an even-numbered SEQ ID NO. of SEQ ID NOs: 4-218, wherein the amino acid sequence comprises one or more substitutions relative to the reference sequence corresponding to residues 12 to 346 of SEQ ID NO: 2, or relative to the reference sequence corresponding to SEQ ID NO: 2.

In some embodiments, the recombinant polynucleotide comprises a polynucleotide sequence encoding an engineered RNA ligase comprising an amino acid sequence comprising at least a substitution at amino acid position 10, 11, 12, 13, 15, 17, 18, 21, 23, 25, 26, 28, 29, 30, 32, 33, 37, 38, 39, 40, 41, 42, 45, 52, 54, 56, 57, 58, 60, 61, 62, 64, 65, 67, 71, 73, 74, 75, 77, 80, 81, 83, 84, 86, 87, 89, 90, 91, 93, 95, 97, 99, 100, 101, 102, 103, 104, 105, 106, 107, 109, 113, 114, 116, 117, 119, 121, 122, 123, 125, 126, 127, 128, 129, 131, 135, 137, 140, 141, 143, 144, 148, 150, 154, 156, 157, 158, 159, 160, 161, 162, 165, 169, 170, 171, 173, 175, 177, 178, 179, 181, 183, 184, 185, 190, 191, 192, 193, 194, 197, 198, 204, 206, 208, 210, 212, 213, 214, 215, 218, 223, 224, 226, 228, 229, 230, 232, 236, 238, 241, 243, 247, 248, 249, 250, 251, 252, 253, 254, 255, 256, 257, 258, 260, 261, 264, 265, 266, 267, 268, 270, 271, 272, 274, 276, 277, 280, 282, 283, 286, 288, 289, 290, 291, 292, 294, 295, 296, 297, 298, 299, 300, 301, 302, 305, 306, 307, 310, 311, 315, 316, 317, 318, 323, 324, 325, 326, 327, 330, 331, 332, 334, 335, 336, 337, 338, 341, 342, 344, 345, or 346, or any combinations thereof, wherein the amino acid positions are relative to the reference sequence corresponding to residues 12 to 346 of SEQ ID NO: 2, or relative to the reference sequence corresponding to SEQ ID NO: 2.

In some embodiments, the recombinant polynucleotide comprises a polynucleotide sequence encoding an engineered RNA ligase comprising an amino acid sequence comprising at least a substitution at amino acid position 10, 11, 12, 13, 17, 21, 23, 25, 26, 28, 29, 30, 32, 37, 38, 40, 41, 42, 45, 52, 54, 56, 57, 58, 60, 61, 62, 64, 65, 67, 71, 73, 74, 75, 77, 80, 83, 84, 87, 90, 91, 93, 97, 99, 100, 101, 102, 103, 104, 105, 106, 107, 109, 113, 114, 121, 122, 123, 125, 126, 127, 128, 129, 131, 135, 137, 140, 141, 143, 148, 150, 157, 158, 160, 161, 162, 165, 169, 170, 173, 178, 179, 184, 190, 191, 192, 193, 194, 197, 198, 204, 206, 208, 210, 213, 215, 218, 223, 228, 229, 232, 236, 238, 241, 243, 248, 249, 250, 251, 252, 253, 254, 255, 258, 261, 264, 265, 267, 268, 270, 271, 272, 274, 276, 277, 282, 286, 289, 290, 292, 294, 295, 297, 298, 299, 300, 301, 302, 305, 311, 318, 323, 324, 325, 327, 332, 336, 338, 341, 344, or 346; a substitution or amino acid residue 18A/H, 33A/I/T/V, 39P, 86D/L, 89C/D/E/L/N/Q/R/S/T/V/Y, 95A/C/G/I, 116L, 117A/Q, 119L, 144L, 154S/V, 156G/H/S, 171S, 175A, 177K, 181E/N/P/R/S, 183L, 212E/G/K/L, 224A/Q/R, 226R, 230T/V, 247I/L/R/S, 256E/G/M/S/W, 257D/E/G, 260G, 280L/W, 283C/V, 288L/R/W, 291S, 296E/G/L/Q/W, 306G/T, 307K/R, 310H/L/M/N/Q/R/S/T, 315E, 316I/Y, 317E/F/G/Q/V/W, 326C/L, 330A/D/E/G/K, 331D/G/S, 334E, 335Q, 337A/V, 338G/L/M, 342R/S/T, or 345Q/S; or any combinations thereof, wherein the amino acid positions are relative to the reference sequence corresponding to residues 12 to 346 of SEQ ID NO: 2, or relative to the reference sequence corresponding to SEQ ID NO: 2.

In some embodiments, the recombinant polynucleotide comprises a polynucleotide sequence encoding an engineered RNA ligase comprising an amino acid sequence comprising at least a substitution at amino acid position 11, 12, 23, 25, 29, 37, 38, 75, 86, 89, 93, 101, 122, 143, 157, 183, 192, 194, 198, 212, 214, 230, 241, 248, 249, 250, 251, 254, 266, 268, 280, 291, 295, 298, 307, 316, 318, 326, 330, or 331, or any combinations thereof, wherein the amino acid positions are relative to the reference sequence corresponding to residues 12 to 346 of SEQ ID NO: 2, or relative to the reference sequence corresponding to SEQ ID NO: 2.

In some embodiments, the recombinant polynucleotide comprises a polynucleotide sequence encoding an engineered RNA ligase comprising an amino acid sequence comprising at least a substitution at amino acid position 11, 12, 23, 25, 29, 37, 38, 75, 93, 101, 122, 143, 157, 192, 194, 198, 241, 248, 249, 250, 251, 254, 268, 295, 298, or 318; a substitution or amino acid residue 86D/L, 89C/D/E/L/N/Q/R/S/T/V/Y, 183L, 212E/G/K/L, 214A/C/H/K/L/Q/Y, 226R, 230T/V, 266C/1, 280L/W, 291H/L/Q/R/S/T/W, 307K/R, 326C/L, 330A/D/E/G/K, or 331D/G/S; or any combinations thereof, wherein the amino acid positions are relative to the reference sequence corresponding to residues 12 to 346 of SEQ ID NO: 2, or relative to the reference sequence corresponding to SEQ ID NO: 2.

In some embodiments, the recombinant polynucleotide comprises a polynucleotide sequence encoding an engineered RNA ligase comprising an amino acid sequence comprising at least a substitution or substitution set at amino acid position(s) 256, 258, 254, 251, 248, 37, 54, 232, 15, 71, 26, 116, 38, 25, 106/251, 52, 33, 247, 37/249/266/316/330/334/335, 21, 11/37/249/266/316, 261, 11/266/316, 11/248/249/266, 331, 11/248/249, 255, 37/249/266/316/334, 266, 11/37/156/249/335, 37/156/248/249/266/334, 334, 156, 11/266/316/334, 11/39/156/248/316, 11/249, 37/156,107/251, 156/249/316, 37/156/316, 11/37/156/249, 11/37/334, 194, 37/316,175, 11/156/249/316, 11/37, 252, 12, 37/266, 11/37/156/266, 11/37/156/248/266, 37/236/248/249, 37/156/248/249, 11/266/330/335, 249, 11/156/248/249/316, 37/249/316, 11, 11/156, 11/37/249, 330, 316, 156/249/266/335, 249/266/316/330/335, 11/156/249, 266/316/330/334/335, 156/248/249/316/334, 37/156/248/249/316, 156/266, 248/249, 156/249/316/330/335, 218, 37/156/248/249/266, 11/37/156/248/249/316, 37/156/316/330, 11/37/248/249/316/330, 11/37/248/249, 249/330/335, 11/37/156/330/334, 40, 11/37/148/249/316, 83, 323, 193, 335, or 257, wherein the amino acid positions are relative to the reference sequence corresponding to residues 12 to 346 of SEQ ID NO: 2, or relative to the reference sequence corresponding to SEQ ID NO: 2.

In some embodiments, the recombinant polynucleotide comprises a polynucleotide sequence encoding an engineered RNA ligase comprising an amino acid sequence comprising at least a substitution at an amino acid position provided in Tables 5.1, 6.1, 6.2, 7.1, 8.1, 8.2, 9.1, 10.1, 11.1, 12.1, 13.1, 14.1, 15.1, 16.1, 17.1, 18.1, 19.1, 20.1, 21.1, 22.1, 23.1, 24.1, 25.1, and 26.1, wherein the amino acid positions are relative to the reference sequence corresponding to residues 12 to 346 of SEQ ID NO: 2, or relative to the reference sequence corresponding to SEQ ID NO: 2.

In some embodiments, the recombinant polynucleotide comprises a polynucleotide sequence encoding an engineered RNA ligase comprising an amino acid sequence comprising at least one substitution provided in Tables 5.1, 6.1, 6.2, 7.1, 8.1, 8.2, 9.1, 10.1, 11.1, 12.1, 13.1, 14.1, 15.1, 16.1, 17.1, 18.1, 19.1, 20.1, 21.1, 22.1, 23.1, 24.1, 25.1, and 26.1, wherein the amino acid positions are relative to the reference sequence corresponding to residues 12 to 346 of SEQ ID NO: 2, or relative to the reference sequence corresponding to SEQ ID NO: 2.

In some embodiments, the recombinant polynucleotide comprises a polynucleotide sequence encoding an engineered RNA ligase comprising an amino acid sequence comprising at least a substitution or substitution set at amino acid position(s) provided in Tables 5.1, 6.1, 6.2, 7.1, 8.1, 8.2, 9.1, 10.1, 11.1, 12.1, 13.1, 14.1, 15.1, 16.1, 17.1, 18.1, 19.1, 20.1, 21.1, 22.1, 23.1, 24.1, 25.1, and 26.1, wherein the amino acid positions are relative to the reference sequence corresponding to residues 12 to 346 of SEQ ID NO: 2, or relative to the reference sequence corresponding to SEQ ID NO: 2.

In some embodiments, the recombinant polynucleotide comprises a polynucleotide sequence encoding an engineered RNA ligase comprising an amino acid sequence comprising at least a substitution or substitution set of an RNA ligase variant provided in Tables 5.1, 6.1, 6.2, 7.1, 8.1, 8.2, 9.1, 10.1, 11.1, 12.1, 13.1, 14.1, 15.1, 16.1, 17.1, 18.1, 19.1, 20.1, 21.1, 22.1, 23.1, 24.1, 25.1, and 26.1, wherein the amino acid positions are relative to the reference sequence corresponding to residues 12 to 346 of SEQ ID NO: 2, or relative to the reference sequence corresponding to SEQ ID NO: 2.

In some embodiments, the recombinant polynucleotide comprises a polynucleotide sequence encoding an engineered RNA ligase comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to a reference sequence comprising a substitution or substitution set of an RNA ligase variant provided in Tables 5.1, 6.1, 6.2, 7.1, 8.1, 8.2, 9.1, 10.1, 11.1, 12.1, 13.1, 14.1, 15.1, 16.1, 17.1, 18.1, 19.1, 20.1, 21.1, 22.1, 23.1, 24.1, 25.1, and 26.1, wherein the amino acid positions are relative to the reference sequence corresponding to residues 12 to 346 of SEQ ID NO: 2, or relative to the reference sequence corresponding to SEQ ID NO: 2.

In some embodiments, the recombinant polynucleotide comprises a polynucleotide sequence encoding an engineered RNA ligase comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12 to 346 of SEQ ID NO: 54, 244, 648, 862, 956, 1088, 1416, 1612, 1840, 1866, 1954, 2062, 2674, 2754, or 2786, or to the reference sequence corresponding to SEQ ID NO: 54, 244, 648, 862, 956, 1088, 1416, 1612, 1840, 1866, 1954, 2062, 2674, 2754, or 2786.

In some embodiments, the recombinant polynucleotide comprises a polynucleotide sequence encoding an engineered RNA ligase comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12 to 346 of an even-numbered SEQ ID NO. of SEQ ID NOs: 4-1470 and 1478-3058, or to the reference sequence corresponding to an even-numbered SEQ ID NO. of SEQ ID NOs: 4-1470 and 1478-3058.

In some embodiments, the recombinant polynucleotide comprises a polynucleotide sequence encoding an engineered RNA ligase comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12 to 346 of SEQ ID NO: 54, 244, 648, 862, 956, 1088, 1416, 1612, 1840, 1866, 1954, 2062, 2674, 2754, or 2786, or to the reference sequence corresponding to SEQ ID NO: 54, 244, 648, 862, 956, 1088, 1416, 1612, 1840, 1866, 1954, 2062, 2674, 2754, or 2786, wherein the amino acid sequence comprises one or more substitutions relative to the reference sequence corresponding to residues 12 to 346 of SEQ ID NO: 54, 244, 648, 862, 956, 1088, 1416, 1612, 1840, 1866, 1954, 2062, 2674, 2754, or 2786, or relative to the reference sequence corresponding to SEQ ID NO: 54, 244, 648, 862, 956, 1088, 1416, 1612, 1840, 1866, 1954, 2062, 2674, 2754, or 2786.

In some embodiments, the recombinant polynucleotide comprises a polynucleotide sequence encoding an engineered RNA ligase comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12 to 346 of an even-numbered SEQ ID NO. of SEQ ID NOs: 4-1470 and 1478-3058, or to the reference sequence corresponding to an even-numbered SEQ ID NO. of SEQ ID NOs: 4-1470 and 1478-3058, wherein the amino acid sequence comprises one or more substitutions relative to the reference sequence corresponding to residues 12 to 346 of SEQ ID NO: 54, 244, 648, 862, 956, 1088, 1416, 1612, 1840, 1866, 1954, 2062, 2674, 2754, or 2786, or relative to the reference sequence corresponding to SEQ ID NO: 54, 244, 648, 862, 956, 1088, 1416, 1612, 1840, 1866, 1954, 2062, 2674, 2754, or 2786.

In some embodiments, the recombinant polynucleotide comprises a polynucleotide sequence encoding an engineered RNA ligase comprising an amino acid sequence comprising at least a substitution at amino acid position 10, 11, 12, 13, 15, 17, 18, 21, 23, 25, 26, 28, 29, 30, 32, 33, 37, 38, 39, 40, 41, 42, 45, 52, 54, 56, 57, 58, 60, 61, 62, 64, 65, 67, 71, 73, 74, 75, 77, 80, 81, 83, 84, 86, 87, 89, 90, 91, 93, 95, 97, 99, 100, 101, 102, 103, 104, 105, 106, 107, 109, 113, 114, 116, 117, 119, 121, 122, 123, 125, 126, 127, 128, 129, 131, 135, 137, 140, 141, 143, 144, 148, 150, 154, 156, 157, 158, 159, 160, 161, 162, 165, 169, 170, 171, 173, 175, 177, 178, 179, 181, 183, 184, 185, 190, 191, 192, 193, 194, 197, 198, 204, 206, 208, 210, 212, 213, 214, 215, 218, 223, 224, 226, 228, 229, 230, 232, 236, 238, 241, 243, 247, 248, 249, 250, 251, 252, 253, 254, 255, 256, 257, 258, 260, 261, 264, 265, 266, 267, 268, 270, 271, 272, 274, 276, 277, 280, 282, 283, 286, 288, 289, 290, 291, 292, 294, 295, 296, 297, 298, 299, 300, 301, 302, 305, 306, 307, 310, 311, 315, 316, 317, 318, 323, 324, 325, 326, 327, 330, 331, 332, 334, 335, 336, 337, 338, 341, 342, 344, 345, or 346, or any combinations thereof, wherein the amino acid positions are relative to the reference sequence corresponding to residues 12 to 346 of SEQ ID NO: 54, 244, 648, 862, 956, 1088, 1416, 1612, 1840, 1866, 1954, 2062, 2674, 2754, or 2786, or relative to the reference sequence corresponding to SEQ ID NO: 54, 244, 648, 862, 956, 1088, 1416, 1612, 1840, 1866, 1954, 2062, 2674, 2754, or 2786.

In some embodiments, the recombinant polynucleotide comprises a polynucleotide sequence encoding an engineered RNA ligase comprising an amino acid sequence comprising at least a substitution at amino acid position 10, 11, 12, 13, 17, 21, 23, 25, 26, 28, 29, 30, 32, 37, 38, 40, 41, 42, 45, 52, 54, 56, 57, 58, 60, 61, 62, 64, 65, 67, 71, 73, 74, 75, 77, 80, 83, 84, 87, 90, 91, 93, 97, 99, 100, 101, 102, 103, 104, 105, 106, 107, 109, 113, 114, 121, 122, 123, 125, 126, 127, 128, 129, 131, 135, 137, 140, 141, 143, 148, 150, 157, 158, 160, 161, 162, 165, 169, 170, 173, 178, 179, 184, 190, 191, 192, 193, 194, 197, 198, 204, 206, 208, 210, 213, 215, 218, 223, 228, 229, 232, 236, 238, 241, 243, 248, 249, 250, 251, 252, 253, 254, 255, 258, 261, 264, 265, 267, 268, 270, 271, 272, 274, 276, 277, 282, 286, 289, 290, 292, 294, 295, 297, 298, 299, 300, 301, 302, 305, 311, 318, 323, 324, 325, 327, 332, 336, 338, 341, 344, or 346; a substitution or amino acid residue 18A/H, 33A/I/T/V, 39P, 86D/L, 89C/D/E/L/N/Q/R/S/T/V/Y, 95A/C/G/I, 116L, 117A/Q, 119L, 144L, 154S/V, 156G/H/S, 171S, 175A, 177K, 181E/N/P/R/S, 183L, 212E/G/K/L, 224A/Q/R, 226R, 230T/V, 247I/L/R/S, 256E/G/M/S/W, 257D/E/G, 260G, 280L/W, 283C/V, 288L/R/W, 291S, 296E/G/L/Q/W, 306G/T, 307K/R, 310H/L/M/N/Q/R/S/T, 315E, 316I/Y, 317E/F/G/Q/V/W, 326C/L, 330A/D/E/G/K, 331D/G/S, 334E, 335Q, 337A/V, 338G/L/M, 342R/S/T, or 345Q/S; or any combinations thereof, wherein the amino acid positions are relative to the reference sequence corresponding to residues 12 to 346 of SEQ ID NO: 54, 244, 648, 862, 956, 1088, 1416, 1612, 1840, 1866, 1954, 2062, 2674, 2754, or 2786, or relative to the reference sequence corresponding to SEQ ID NO: 54, 244, 648, 862, 956, 1088, 1416, 1612, 1840, 1866, 1954, 2062, 2674, 2754, or 2786.

In some embodiments, the recombinant polynucleotide comprises a polynucleotide sequence encoding an engineered RNA ligase comprising an amino acid sequence comprising at least a substitution at amino acid position 11, 12, 23, 25, 29, 37, 38, 75, 86, 89, 93, 101, 122, 143, 157, 183, 192, 194, 198, 212, 214, 230, 241, 248, 249, 250, 251, 254, 266, 268, 280, 291, 295, 298, 307, 316, 318, 326, 330, or 331, or any combinations thereof, wherein the amino acid positions are relative to the reference sequence corresponding to residues 12 to 346 of SEQ ID NO: 54, 244, 648, 862, 956, 1088, 1416, 1612, 1840, 1866, 1954, 2062, 2674, 2754, or 2786, or relative to the reference sequence corresponding to SEQ ID NO: 54, 244, 648, 862, 956, 1088, 1416, 1612, 1840, 1866, 1954, 2062, 2674, 2754, or 2786.

In some embodiments, the recombinant polynucleotide comprises a polynucleotide sequence encoding an engineered RNA ligase comprising an amino acid sequence comprising at least a substitution at amino acid position 11, 12, 23, 25, 29, 37, 38, 75, 93, 101, 122, 143, 157, 192, 194, 198, 241, 248, 249, 250, 251, 254, 268, 295, 298, or 318; a substitution or amino acid residue 86D/L, 89C/D/E/L/N/Q/R/S/T/V/Y, 183L, 212E/G/K/L, 214A/C/H/K/L/Q/Y, 226R, 230T/V, 266C/I, 280L/W, 291H/L/Q/R/S/T/W, 307K/R, 326C/L, 330A/D/E/G/K, or 331D/G/S; or any combinations thereof, wherein the amino acid positions are relative to the reference sequence corresponding to residues 12 to 346 of SEQ ID NO: 54, 244, 648, 862, 956, 1088, 1416, 1612, 1840, 1866, 1954, 2062, 2674, 2754, or 2786, or relative to the reference sequence corresponding to SEQ ID NO: 54, 244, 648, 862, 956, 1088, 1416, 1612, 1840, 1866, 1954, 2062, 2674, 2754, or 2786.

In some embodiments, the recombinant polynucleotide comprises a polynucleotide sequence encoding an engineered RNA ligase further comprising an amino acid sequence comprising at least a substitution at amino acid position 2, 14, 15, 63, 69, 81, 85, 95, 138, 142, 149, 159, 171, 186, 195, 202, 266, 275, 283, 285, 303, 314, 333, or 339; a substitution or amino acid residue 181, 33L, 39Q/S, 86G, 89G/H/I/M/W, 116N, 117D, 119T, 144W, 154C, 156A/L/N/Q/T, 175K, 177P, 181D, 183V, 185G/K, 212I, 2141, 224Y, 226I, 230D, 247K, 256A, 257L, 260S, 280G/N, 288E, 291P, 296K, 306L, 307E/Q, 310K, 315S/T, 316L, 317A, 326R, 330M/R, 331L/R/W, 334R, 335D/H, 337L, 3421, or 345V; or any combinations thereof, wherein the amino acid positions are relative to the reference sequence corresponding to residues 12 to 346 of SEQ ID NO: 54, 244, 648, 862, 956, 1088, 1416, 1612, 1840, 1866, 1954, 2062, 2674, 2754, or 2786, or relative to the reference sequence corresponding to SEQ ID NO: 54, 244, 648, 862, 956, 1088, 1416, 1612, 1840, 1866, 1954, 2062, 2674, 2754, or 2786.

In some embodiments, the recombinant polynucleotide comprises a polynucleotide sequence encoding an engineered RNA ligase comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12 to 346 of SEQ ID NO: 54, or to the reference sequence corresponding to SEQ ID NO: 54, wherein the amino acid sequence comprises one or more substitutions relative to the reference sequence corresponding to residues 12 to 346 of SEQ ID NO: 54, or relative to the reference sequence corresponding to SEQ ID NO: 54.

In some embodiments, the recombinant polynucleotide comprises a polynucleotide sequence encoding an engineered RNA ligase comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12 to 346 of an even-numbered SEQ ID NO. of SEQ ID NOs: 220-630, or to the reference sequence corresponding to an even-numbered SEQ ID NO. of SEQ ID NOs: 220-630, wherein the amino acid sequence comprises one or more substitutions relative to the reference sequence corresponding to residues 12 to 346 of SEQ ID NO: 54, or relative to the reference sequence corresponding to SEQ ID NO: 54.

In some embodiments, the recombinant polynucleotide comprises a polynucleotide sequence encoding an engineered RNA ligase comprising an amino acid sequence comprising at least a substitution or substitution set at amino acid position(s) 156/232/334/335, 12/254/256, 12/230, 12/230/251, 12/230/251/254/256, 12/38/230, 230/251, 12/38/254, 230/256, 254, 230, 12/230/254, 12/38/230/251/256, 12/251, 12/251/254/256, 38, 12/230/256, 12, 12/25/38/230/251, 12/38/102/230/251/256, 12/256, 38/230/256, 256, 12/230/251/256, 230/251/254, 230/254, 251/254, 12/25/230, 38/230, 38/251, 12/38/230/256, 102, 251, 12/25/251/256, 12/102/256, 12/38/230/251/254/256, 12/251/256, 102/230/251/254/256, 12/38/251, 12/230/251/254, 156/248/334/335, 12/25/38, 12/102/230/251/254, 12/38/251/254, 12/251/254, 12/102, 38/230/251/254/256, 12/25/230/251/256, 12/230/311, 12/254, 38/230/251/254, 12/38/251/256, 156/334/335, 257/258/334, 12/25/38/251/256, 334, 12/25/230/254/256, 38/251/256, 12/38/230/251/254, 12/38, 257/258/334/335, 12/38/254/256, 232/248/257/258/334/335, 156/258/334/335, 156/249/334/335, 12/102/230/254, 25, 156/257/258/334/335, 12/102/230, 156/248/257/258/334, 248/249/334/335, 232/257/258/334/335, 334/335, 156/334, 12/38/102/256, 156/257/334/335, 12/140/251/254, 251/254/256, 248/334/335, 156/258/334, 156/248/330/335, 12/38/256, 232/334/335, 156/249/334, 330/334/335, 156/248/334, 156/335, 257/334, 248/334, 232/248/334/335, 12/25/38/230/251/256, 156/248/249/334/335, 330/335, 248/257/334/335, 232/248/334, 156/232/248/334/335, 232/257/330/334, 156/248/291/334/335, 156/232/248/257/258/334, 156/232/249/334/335, 12/25, 156/248/330/334/335, 156/248/249/334, 232/248/330/335, 156/232/330/334/335, 197, 338, 327, 192, 331, 326, 345, 101, 100, 141, 198, 64, 306, 317, 265, 296, 194, 62, 90, 307, 103, 210, 105, or 61, wherein the amino acid positions are relative to the reference sequence corresponding to residues 12 to 346 of SEQ ID NO: 54, or relative to the reference sequence corresponding to SEQ ID NO: 54.

In some embodiments, the recombinant polynucleotide comprises a polynucleotide sequence encoding an engineered RNA ligase comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12 to 346 of SEQ ID NO: 244, or to the reference sequence corresponding to SEQ ID NO: 244, wherein the amino acid sequence comprises one or more substitutions relative to the reference sequence corresponding to residues 12 to 346 of SEQ ID NO: 244, or relative to the reference sequence corresponding to SEQ ID NO: 244.

In some embodiments, the recombinant polynucleotide comprises a polynucleotide sequence encoding an engineered RNA ligase comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12 to 346 of an even-numbered SEQ ID NO. of SEQ ID NOs: 632-820, or to the reference sequence corresponding to an even-numbered SEQ ID NO. of SEQ ID NOs: 632-820, wherein the amino acid sequence comprises one or more substitutions relative to the reference sequence corresponding to residues 12 to 346 of SEQ ID NO: 244, or relative to the reference sequence corresponding to SEQ ID NO: 244.

In some embodiments, the recombinant polynucleotide comprises a polynucleotide sequence encoding an engineered RNA ligase comprising an amino acid sequence comprising at least a substitution or substitution set at amino acid position(s) 141/197/326/327, 100/101/338, 141/198/326, 64/100/101, 100/338, 101/141/198/326, 101/198/326/327, 198/326, 64/100/101/338/345, 197/326, 198/327, 141/198/327, 198/326/327, 197/326/327, 64/100/338, 64/100/101/156/197/338, 64/100/101/338, 100/197/338, 197/338, 101/197/326, 64/100/197/338, 101/198, 141/197/327, 64/100/101/197/338, 101/326, 101/197/326/327, 197/327, 197, 141/197, 101, 101/198/326, 64/197/198/338, 141, 101/141, 100/101/197/338, 141/326, 338, 326, 197/198/338, 64/101/338, 64/197/198, 101/197, 100/101, 100/109/197/338, 101/197/338, 197/198, 141/326/327, 100/197, 64/100, 64/197/338, 100/197/198, 101/338, 141/327, 101/306, 64/100/101/197, 101/326/327, 100, 327, 326/345, 64/100/101/197/198, 326/327, 64/101,100/101/197, 101/327, 64/100/197/198/338, 306, 64/338,101/141/326/327, 64/101/197/338, or 101/198/306/327, wherein the amino acid positions are relative to the reference sequence corresponding to residues 12 to 346 of SEQ ID NO: 244, or relative to the reference sequence corresponding to SEQ ID NO: 244.

In some embodiments, the recombinant polynucleotide comprises a polynucleotide sequence encoding an engineered RNA ligase comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12 to 346 of SEQ ID NO: 648, or to the reference sequence corresponding to SEQ ID NO: 648, wherein the amino acid sequence comprises one or more substitutions relative to the reference sequence corresponding to residues 12 to 346 of SEQ ID NO: 648, or relative to the reference sequence corresponding to SEQ ID NO: 648.

In some embodiments, the recombinant polynucleotide comprises a polynucleotide sequence encoding an engineered RNA ligase comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12 to 346 of an even-numbered SEQ ID NO. of SEQ ID NOs: 822-954, or to the reference sequence corresponding to an even-numbered SEQ ID NO. of SEQ ID NOs: 822-954, wherein the amino acid sequence comprises one or more substitutions relative to the reference sequence corresponding to residues 12 to 346 of SEQ ID NO: 648, or relative to the reference sequence corresponding to SEQ ID NO: 648.

In some embodiments, the recombinant polynucleotide comprises a polynucleotide sequence encoding an engineered RNA ligase comprising an amino acid sequence comprising at least a substitution or substitution set at amino acid position(s) 194/330/331, 298, 331, 192/193/194/298, 330, 192/330/331, 192/193/194/331, 298/330/331, 194/298/330/331, 192/193/330/331, 192/298/331, 192/193, 194/330, 60/100/156, 193/296/331, 298/331, 193/194/330/331, 194, 194/298/331, 193/298, 193/330, 192, 192/193/194/298/331, 192/193/194/298/330, 192/193/298/331, 100/156, 62/100,298/330, 60/100, 192/193/298/330/331, 100, 193/194/298, 338, 62, 247, 18, 37, 254, 33, 261, 251, 260, 12, 117, 256, 255, 38, 29, 25, or 252, wherein the amino acid positions are relative to the reference sequence corresponding to residues 12 to 346 of SEQ ID NO: 648, or relative to the reference sequence corresponding to SEQ ID NO: 648.

In some embodiments, the recombinant polynucleotide comprises a polynucleotide sequence encoding an engineered RNA ligase comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12 to 346 of SEQ ID NO: 862, or to the reference sequence corresponding to SEQ ID NO: 862, wherein the amino acid sequence comprises one or more substitutions relative to the reference sequence corresponding to residues 12 to 346 of SEQ ID NO: 862, or relative to the reference sequence corresponding to SEQ ID NO: 862.

In some embodiments, the recombinant polynucleotide comprises a polynucleotide sequence encoding an engineered RNA ligase comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12 to 346 of an even-numbered SEQ ID NO. of SEQ ID NOs: 956-1078, or to the reference sequence corresponding to an even-numbered SEQ ID NO. of SEQ ID NOs: 956-1078, wherein the amino acid sequence comprises one or more substitutions relative to the reference sequence corresponding to residues 12 to 346 of SEQ ID NO: 862, or relative to the reference sequence corresponding to SEQ ID NO: 862.

In some embodiments, the recombinant polynucleotide comprises a polynucleotide sequence encoding an engineered RNA ligase comprising an amino acid sequence comprising at least a substitution or substitution set at amino acid position(s) 254, 18/254,247/317, 37/317/342, 33, 18/156/254, 18, 156, 254/338, 254/307, 33/37/317, 37, 37/156/317, 156/265/317, 265/317, 156/254, 33/156/317, 317, 156/317, 37/317, 18/338,338, 156/247/317, 33/317, 37/247/265/317, 33/37/156/317, 156/247/296/317, 18/307,156/296/317, 156/247, 37/247/317, 18/254/307, 307, 265, 33/37, 18/156, 33/156/296/317, 18/156/254/338, 37/265/317, 33/37/156/247/317, 296, 33/296/317, 156/296, 247/296/317, 37/296/317, 247/296, 33/37/156, 296/317, 156/254/307, 18/156/307, or 33/37/265, wherein the amino acid positions are relative to the reference sequence corresponding to residues 12 to 346 of SEQ ID NO: 862, or relative to the reference sequence corresponding to SEQ ID NO: 862.

In some embodiments, the recombinant polynucleotide comprises a polynucleotide sequence encoding an engineered RNA ligase comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12 to 346 of SEQ ID NO: 956, or to the reference sequence corresponding to SEQ ID NO: 956, wherein the amino acid sequence comprises one or more substitutions relative to the reference sequence corresponding to residues 12 to 346 of SEQ ID NO: 956, or relative to the reference sequence corresponding to SEQ ID NO: 956.

In some embodiments, the recombinant polynucleotide comprises a polynucleotide sequence encoding an engineered RNA ligase comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12 to 346 of an even-numbered SEQ ID NO. of SEQ ID NOs: 1080-1374, or to the reference sequence corresponding to an even-numbered SEQ ID NO. of SEQ ID NOs: 1080-1374, wherein the amino acid sequence comprises one or more substitutions relative to the reference sequence corresponding to residues 12 to 346 of SEQ ID NO: 956, or relative to the reference sequence corresponding to SEQ ID NO: 956.

In some embodiments, the recombinant polynucleotide comprises a polynucleotide sequence encoding an engineered RNA ligase comprising an amino acid sequence comprising at least a substitution or substitution set at amino acid position(s) 12/25/29/156, 12/255/256/257, 12/156/255, 247/252, 12/25, 12, 12/25/29/156/255, 256, 12/156, 12/25/156/255/256/257/261, 18/247/251, 25/255/256/257, 12/25/257, 255/257, 12/25/29/255/257, 256/257, 12/255/257, 12/156/255/256/257, 25, 18/38/251/252, 12/256/257/261, 12/29, 12/25/255/256/257/261, 38, 12/25/29/256/257, 29/257, 12/261,251, 249/251, 12/255, 12/29/156/256/257, 252, 255/257/261, 18, 29/255/257, 255/256/257, 12/25/29/156/256/257, 25/255/256/257/261, 29/255/256/257/261, 25/256, 18/38/247/252/327, 18/247, 25/29, 12/25/156/255/257, 18/156/251/252, 12/255/261, 25/255, 12/255/256/261, 156/255/257, 156/257, 12/156/256/257, 25/156/256/257, 12/25/29/156/257/261, 25/261, 12/156/255/256/261, 12/25/29/255/256/257, 38/252,255, 249/252, 12/156/255/256, 12/25/29/261, 257, 12/255/256, 12/29/255, 12/255/257/261, 12/156/257, 192/249/251/252, 12/25/29/255/261, 12/156/255/257, 12/29/255/257, 12/29/256/257, 18/252,249, 156/250, 38/251/252, 156/255/256/257, 25/257, 12/255/256/257/261, 25/156/261, 156/252, 12/25/255/256/257, 12/25/29/156/255/257, 29/156,251/252, 256/257/261, 25/156/255/256/257, 156/249/251/252, 25/29/255, 12/29/156, 255/256, 38/250/252, 247/249/252, 12/256/257, 60, 12/256, 12/156/256, 25/255/257/261, 12/25/29/255/256/261, 12/29/156/255/256, 12/25/156/255/256, 247/251/252, 256/261, 38/251, 12/25/29/156/261, 12/257,156/255/256/261, 12/25/156/255, 12/25/29/156/255/257/261, 12/29/256/261, 29/156/261, 25/29/156/257/261, 12/25/255/256/261, or 12/156/255/261, wherein the amino acid positions are relative to the reference sequence corresponding to residues 12 to 346 of SEQ ID NO: 956, or relative to the reference sequence corresponding to SEQ ID NO: 956.

In some embodiments, the recombinant polynucleotide comprises a polynucleotide sequence encoding an engineered RNA ligase comprising an amino acid sequence comprising at least a substitution at amino acid position 157, 183, 95, 185, 181, 213, 89, 28, 190, 214, or 143, or any combinations thereof, wherein the amino acid positions are relative to the reference sequence corresponding to residues 12 to 346 of SEQ ID NO: 956, or relative to the reference sequence corresponding to SEQ ID NO: 956.

In some embodiments, the recombinant polynucleotide comprises a polynucleotide sequence encoding an engineered RNA ligase comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12 to 346 of SEQ ID NO: 1088, or to the reference sequence corresponding to SEQ ID NO: 1088, wherein the amino acid sequence comprises one or more substitutions relative to the reference sequence corresponding to residues 12 to 346 of SEQ ID NO: 1088, or relative to the reference sequence corresponding to SEQ ID NO: 1088.

In some embodiments, the recombinant polynucleotide comprises a polynucleotide sequence encoding an engineered RNA ligase comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12 to 346 of an even-numbered SEQ ID NO. of SEQ ID NOs: 1376-1434, or to the reference sequence corresponding to an even-numbered SEQ ID NO. of SEQ ID NOs: 1376-1434, wherein the amino acid sequence comprises one or more substitutions relative to the reference sequence corresponding to residues 12 to 346 of SEQ ID NO: 1088, or relative to the reference sequence corresponding to SEQ ID NO: 1088.

In some embodiments, the recombinant polynucleotide comprises a polynucleotide sequence encoding an engineered RNA ligase comprising an amino acid sequence comprising at least a substitution or substitution set at amino acid position(s) 95/157, 95/183/185/214, 89/183, 28/89/143/183, 28/143/157, 181/183, 143/181/183/214, 143/181/183, 89/143/183, 28/143/181/183, 143/157, 95/181/183, 157, 143/183, 89/143/157/183, 89/95/157, 89/157, 89/95/143/181, 95/143, 89/157/183/214, 143/214, 143/181/183/185, 28/95/143, 143/183/214, 28/95/183/214, 89/143/157, 28/95, 95/143/183/185, or 95/143/157/183, wherein the amino acid positions are relative to the reference sequence corresponding to residues 12 to 346 of SEQ ID NO: 1088, or relative to the reference sequence corresponding to SEQ ID NO: 1088.

In some embodiments, the recombinant polynucleotide comprises a polynucleotide sequence encoding an engineered RNA ligase comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12 to 346 of SEQ ID NO: 1416, or to the reference sequence corresponding to SEQ ID NO: 1416, wherein the amino acid sequence comprises one or more substitutions relative to the reference sequence corresponding to residues 12 to 346 of SEQ ID NO: 1416, or relative to the reference sequence corresponding to SEQ ID NO: 1416.

In some embodiments, the recombinant polynucleotide comprises a polynucleotide sequence encoding an engineered RNA ligase comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12 to 346 of an even-numbered SEQ ID NO. of SEQ ID NOs: 1436-1470, 1478-1766, and 1908-1946, or to the reference sequence corresponding to an even-numbered SEQ ID NO. of SEQ ID NOs: 1436-1470, 1478-1766, and 1908-1946, wherein the amino acid sequence comprises one or more substitutions relative to the reference sequence corresponding to residues 12 to 346 of SEQ ID NO: 1416, or relative to the reference sequence corresponding to SEQ ID NO: 1416.

In some embodiments, the recombinant polynucleotide comprises a polynucleotide sequence encoding an engineered RNA ligase comprising an amino acid sequence comprising at least a substitution or substitution set at amino acid position(s) 83/137/150, 137/213, 137, 150/190, 143/150, 104/150, 60/143/150/190, 60/150/190/213, 104/137/154, 123/137, 137/150/154/190, 123/143/150, 60/104/143/150/213, 60/95/143/190, 123/137/190, 60/123,104/123, or 60/123/143, wherein the amino acid positions are relative to the reference sequence corresponding to residues 12 to 346 of SEQ ID NO: 1416, or relative to the reference sequence corresponding to SEQ ID NO: 1416.

In some embodiments, the recombinant polynucleotide comprises a polynucleotide sequence encoding an engineered RNA ligase comprising an amino acid sequence comprising at least a substitution or substitution set at amino acid position(s) 38, 249, 255, 23, 33, 25, 226, 29, 37, 10, 250, 75, 230, 77, 251, 228, 81, 224, 64, 254, 268, 65, 232, 332, 331, 330, 101, 307, 137, 223, 280, 289, 288, 342, 93, 296, 86, 241, 126, 277, 87, 247, 243, 84, 83, 192, 105, 25/29, 25/29/250, 25/29/75/77/250/255, 25/75/230, 25/77/226/255, 25/230,250/255, 29/75/226/230/255, 29/75/77, 29/75/77/226, 29/75/77/230/255, 29/226, 29/226/230, 29/230/255, 33/228, 33/249, 33/37/228, 33/37/38/228, 33/37/38/228/249, 33/38, 75/250/255, 75/226, 75/226/250/255, 75/226/230/250/255, 75/77, 75/77/255, 75/77/250/255, 75/77/226/255, 75/77/226/250/255, 75/77/230, 75/77/230/250, 75/230, 75/230/255, 75/230/250, 226/255, 226/250, 226/230, 226/230/255, 37/228, 37/228/249, 37/38, 37/38/228, 37/38/228/249, 37/38/249, 38/249/291, 10/228, 10/228/249, 10/33/249, 10/33/37, 10/33/37/38/249, 10/33/38/249, 10/33/38, 10/33/249/251, 10/33/37/228, 10/33/37/249, 10/33/37/38/228/249, 10/33/37/38, 10/249, 10/37, 10/37/228, 10/37/228/249, 10/37/249, 10/37/38/228, 10/37/38/228/249, 10/37/38, 10/38/249, 10/38, 10/38/228, 77/255, 77/250, 77/250/255, 77/226, 77/226/255, 77/230, 77/230/255, 230/255, 230/250, 230/250/255, 23/25/226/255, 23/25/226/230, or 23/25/230, wherein the amino acid positions are relative to the reference sequence corresponding to residues 12 to 346 of SEQ ID NO: 1416, or relative to the reference sequence corresponding to SEQ ID NO: 1416.

In some embodiments, the recombinant polynucleotide comprises a polynucleotide sequence encoding an engineered RNA ligase comprising an amino acid sequence comprising at least a substitution or substitution set at amino acid position(s) 86, 255, 306, 250, 75, 318, 335, 241, 248, 344, 38, 272, 83, 257, 258, or 323, wherein the amino acid positions are relative to the reference sequence corresponding to residues 12 to 346 of SEQ ID NO: 1416, or relative to the reference sequence corresponding to SEQ ID NO: 1416.

In some embodiments, the recombinant polynucleotide comprises a polynucleotide sequence encoding an engineered RNA ligase comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12 to 346 of SEQ ID NO: 1612, or to the reference sequence corresponding to SEQ ID NO: 1612, wherein the amino acid sequence comprises one or more substitutions relative to the reference sequence corresponding to residues 12 to 346 of SEQ ID NO: 1612, or relative to the reference sequence corresponding to SEQ ID NO: 1612.

In some embodiments, the recombinant polynucleotide comprises a polynucleotide sequence encoding an engineered RNA ligase comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12 to 346 of an even-numbered SEQ ID NO. of SEQ ID NOs: 1768-1840, or to the reference sequence corresponding to an even-numbered SEQ ID NO. of SEQ ID NOs: 768-1840, wherein the amino acid sequence comprises one or more substitutions relative to the reference sequence corresponding to residues 12 to 346 of SEQ ID NO: 1612, or relative to the reference sequence corresponding to SEQ ID NO: 1612.

In some embodiments, the recombinant polynucleotide comprises a polynucleotide sequence encoding an engineered RNA ligase comprising an amino acid sequence comprising at least a substitution or substitution set at amino acid position(s) 101, 291, 291/307, 331, 33/75, 33/75/249/291/331, 33/38, 33/93/249, 38/75/291/307, 38/75/249/250, 38/75/93/271, 38/93, 10/75, 10/38/75, 10/38/75/291, 10/38/75/249, 10/38/75/93, 64/224,224, 23/101/307, 23/268, 23/37/64/224/254, 23/64/81, 23/224/268, 23/224/254, 23, 23/64/224, 64, 75/249/307, 93, 33/38/75/249, 75/249, 25/38/75/93/291, 33/38/75, 75, or 23/254, wherein the amino acid positions are relative to the reference sequence corresponding to residues 12 to 346 of SEQ ID NO: 1612, or relative to the reference sequence corresponding to SEQ ID NO: 1612.

In some embodiments, the recombinant polynucleotide comprises a polynucleotide sequence encoding an engineered RNA ligase comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12 to 346 of SEQ ID NO: 1840, or to the reference sequence corresponding to SEQ ID NO: 1840, wherein the amino acid sequence comprises one or more substitutions relative to the reference sequence corresponding to residues 12 to 346 of SEQ ID NO: 1840, or relative to the reference sequence corresponding to SEQ ID NO: 1840.

In some embodiments, the recombinant polynucleotide comprises a polynucleotide sequence encoding an engineered RNA ligase comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12 to 346 of an even-numbered SEQ ID NO. of SEQ ID NOs: 1842-1906, or to the reference sequence corresponding to an even-numbered SEQ ID NO. of SEQ ID NOs: 1842-1906, wherein the amino acid sequence comprises one or more substitutions relative to the reference sequence corresponding to residues 12 to 346 of SEQ ID NO: 1840, or relative to the reference sequence corresponding to SEQ ID NO: 1840.

In some embodiments, the recombinant polynucleotide comprises a polynucleotide sequence encoding an engineered RNA ligase comprising an amino acid sequence comprising at least a substitution or substitution set at amino acid position(s) 38/65/192, 38/65, 38/65/224, 38/64, 93/307, 38, 38/224, 38/192/224, 65, 192/224, 75/307, 75/250/268/307, 250/268/307/342, 224, 65/192,268, 64/192,250/268/291/342, 75/93/249/307/342, 250/291, 38/64/192, 38/64/224, 93/249/307, 38/64/86/192/241, 38/342,248/250/307, 75/77/250/268/307/342, 93/268,192, 38/192, or 64, wherein the amino acid positions are relative to the reference sequence corresponding to residues 12 to 346 of SEQ ID NO: 1840, or relative to the reference sequence corresponding to SEQ ID NO: 1840.

In some embodiments, the recombinant polynucleotide comprises a polynucleotide sequence encoding an engineered RNA ligase comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12 to 346 of SEQ ID NO: 1866, or to the reference sequence corresponding to SEQ ID NO: 1866, wherein the amino acid sequence comprises one or more substitutions relative to the reference sequence corresponding to residues 12 to 346 of SEQ ID NO: 1866, or relative to the reference sequence corresponding to SEQ ID NO: 1866.

In some embodiments, the recombinant polynucleotide comprises a polynucleotide sequence encoding an engineered RNA ligase comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12 to 346 of an even-numbered SEQ ID NO. of SEQ ID NOs: 1948-2038 and 2130-2620, or to the reference sequence corresponding to an even-numbered SEQ ID NO. of SEQ ID NOs: 1948-2038 and 2130-2620, wherein the amino acid sequence comprises one or more substitutions relative to the reference sequence corresponding to residues 12 to 346 of SEQ ID NO: 1866, or relative to the reference sequence corresponding to SEQ ID NO: 1866.

In some embodiments, the recombinant polynucleotide comprises a polynucleotide sequence encoding an engineered RNA ligase comprising an amino acid sequence comprising at least a substitution or substitution set at amino acid position(s) 38/248/251, 38/248/250/251/277, 248/250/251, 38/93/192/248/251, 38/192/248/251, 250/251, 38/248/250/251, 248/251/344, 192/248/250/251, 38/248/250, 38/93/250/251/277, 38/255/280/318, 248, 38/86/241/255/280/318/335, 38/255, 38, 318, 38/101/255/280, 38/318/335, 38/255/318, 192/250/251/344, 38/335,318/335, 38/277, 38/93/251, 192/251, 255, 38/64/318, 38/64/241/255/318, 251, 64/101/255/280/318, 38/101/255/280/318/335, 38/255/280, 38/251/277, 64/318, 38/64/255/318, 241/255/280/318, 38/86/101/241/318, 38/241, 38/255/318/335, 38/64/101/318, 38/64/255/280/318, 241/255/318, or 38/241/335, wherein the amino acid positions are relative to the reference sequence corresponding to residues 12 to 346 of SEQ ID NO: 1866, or relative to the reference sequence corresponding to SEQ ID NO: 1866.

In some embodiments, the recombinant polynucleotide comprises a polynucleotide sequence encoding an engineered RNA ligase comprising an amino acid sequence comprising at least a substitution or substitution set at amino acid position(s) 89, 122, 181, 28, 125, 157, 238, 330, 212, 143, 208, 290, 161, 214, 84, 270, 91, 295, 316, 286, 301, 170, 264, 289, 190, 183, 128, 60, 310, 131, 302, 267, 288, 185, 291, 129, 280, 298, 158/215, 28/122,276, 274, 271, 215, 294, 106, 58, 135, 45, 299, 315, 204, 332, 169, 305, 154, 158, 297, 95, 337, 324, 150, 179, 283, 206, 236, 266, 213, 336, 178, 99, 184, 282, 57, 177, 109, 191, 107, 160, 144, 56, 159, 341, 325, or 91, wherein the amino acid positions are relative to the reference sequence corresponding to residues 12 to 346 of SEQ ID NO: 1866, or relative to the reference sequence corresponding to SEQ ID NO: 1866.

In some embodiments, the recombinant polynucleotide comprises a polynucleotide sequence encoding an engineered RNA ligase comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12 to 346 of SEQ ID NO: 1954, or to the reference sequence corresponding to SEQ ID NO: 1954, wherein the amino acid sequence comprises one or more substitutions relative to the reference sequence corresponding to residues 12 to 346 of SEQ ID NO: 1954, or relative to the reference sequence corresponding to SEQ ID NO: 1954.

In some embodiments, the recombinant polynucleotide comprises a polynucleotide sequence encoding an engineered RNA ligase comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12 to 346 of an even-numbered SEQ ID NO. of SEQ ID NOs: 2040-2128, or to the reference sequence corresponding to an even-numbered SEQ ID NO. of SEQ ID NOs: 2040-2128, wherein the amino acid sequence comprises one or more substitutions relative to the reference sequence corresponding to residues 12 to 346 of SEQ ID NO: 1954, or relative to the reference sequence corresponding to SEQ ID NO: 1954.

In some embodiments, the recombinant polynucleotide comprises a polynucleotide sequence encoding an engineered RNA ligase comprising an amino acid sequence comprising at least a substitution or substitution set at amino acid position(s) 93/232/291/306, 93/232/280/306, 93/241/257/306/335, 232/280, 93/291/306, 93/257, 93/306, 93/280,192/318, 93/280/291, 93/241/291/306, 192, 257/291, 241/257/306, 280/335, 93/291/306/335, 93/335, 93/232/335, 64/318, 93/241, 93/232/241/280, 64/192/318/323, 232/280/306, 93/232,291/335, 257/258, 93/232/241/257/258/280/335, 318/323, 93/241/291/335, 83/86/323, 257/258/291, 101/224/318, 83/192/255/296, 171/232/257/258/280, 83/224/255/318, 64/192/250/318, 101/296, 232/241/280, 64/255/318, 86/250/255, 83/86/101, 83/86, 93/241/258/335, 250/296/323, or 257/258/280, wherein the amino acid positions are relative to the reference sequence corresponding to residues 12 to 346 of SEQ ID NO: 1954, or relative to the reference sequence corresponding to SEQ ID NO: 1954.

In some embodiments, the recombinant polynucleotide comprises a polynucleotide sequence encoding an engineered RNA ligase comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12 to 346 of SEQ ID NO: 2062, or to the reference sequence corresponding to SEQ ID NO: 2062, wherein the amino acid sequence comprises one or more substitutions relative to the reference sequence corresponding to residues 12 to 346 of SEQ ID NO: 2062, or relative to the reference sequence corresponding to SEQ ID NO: 2062.

In some embodiments, the recombinant polynucleotide comprises a polynucleotide sequence encoding an engineered RNA ligase comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12 to 346 of an even-numbered SEQ ID NO. of SEQ ID NOs: 2622-2746, or to the reference sequence corresponding to an even-numbered SEQ ID NO. of SEQ ID NOs: 2622-2746, wherein the amino acid sequence comprises one or more substitutions relative to the reference sequence corresponding to residues 12 to 346 of SEQ ID NO: 2062, or relative to the reference sequence corresponding to SEQ ID NO: 2062.

In some embodiments, the recombinant polynucleotide comprises a polynucleotide sequence encoding an engineered RNA ligase comprising an amino acid sequence comprising at least a substitution or substitution set at amino acid position(s) 83/84/330, 91/270/290, 83/181,161/290/330, 181/330, 161/270/330, 270/290, 83/84/270/318/330, 64/83/84/161/192/290/318, 91/161/290/318/330, 161/330, 161/214/270/290/330, 192/270/318, 91/161/192/330, 161/270, 181/270/330, 91/181/192/290, 161/290, 83/161/270, 83/192/318, 214/330, 83/161/290/330, 64/83/161/290/330, 83/161/192/214/318, 122/143/212/295, 161/192/270/290, 181/290, 91/161/214/270/290/318, 91/192/214/270, 214/290, 64/83/161/270/318/330, 91/161/181/330, 161/318/330, 91/270/290/330, 64/181/290/318, 181/270/290/318, 64/83/161/192/270/290/318/330, 64/83/161/214/290/318, 122/192/212, 64/83/91/192/214/330, 83/91/161/181/290/318, 91/161/192/214/270/290, 91/161/181/290/330, 83/91/125/161/270, 28/212,122/212, 28/64/83/122/143/192/212/295/318, 122/295, 91/161/192/318, 28/122/143/212, 64/161/290, 64/122/208/318, 64/83/192/290/318, 64/83/161/290, 181/192/318, 83/161/192/318, 214/270, 83/122, or 192/212, 28/122, wherein the amino acid positions are relative to the reference sequence corresponding to residues 12 to 346 of SEQ ID NO: 2062, or relative to the reference sequence corresponding to SEQ ID NO: 2062.

In some embodiments, the recombinant polynucleotide comprises a polynucleotide sequence encoding an engineered RNA ligase comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12 to 346 of SEQ ID NO: 2674, or to the reference sequence corresponding to SEQ ID NO: 2674, wherein the amino acid sequence comprises one or more substitutions relative to the reference sequence corresponding to residues 12 to 346 of SEQ ID NO: 2674, or relative to the reference sequence corresponding to SEQ ID NO: 2674.

In some embodiments, the recombinant polynucleotide comprises a polynucleotide sequence encoding an engineered RNA ligase comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12 to 346 of an even-numbered SEQ ID NO. of SEQ ID NOs: 2748-2782, 2822-2914, and 2916-3012, or to the reference sequence corresponding to an even-numbered SEQ ID NO. of SEQ ID NOs: 2748-2782, 2822-2914, and 2916-3012, wherein the amino acid sequence comprises one or more substitutions relative to the reference sequence corresponding to residues 12 to 346 of SEQ ID NO: 2674, or relative to the reference sequence corresponding to SEQ ID NO: 2674.

In some embodiments, the recombinant polynucleotide comprises a polynucleotide sequence encoding an engineered RNA ligase comprising an amino acid sequence comprising at least a substitution or substitution set at amino acid position(s) 28/291/295/298/315, 28/95/291/295/298/315, 143/295/298/315, 291/295/298, 28/143/295/298, 28/291/295/298, 143/185/291/295/298, 28/143/185/291/298/336, 28/298, 280/295, 143/298/336, 143/291/295/298, 291/295/298/315/336, 143/295/298, 28/143/295/298/336, 95/143/295/298, 143/280/290, or 143/291/298, wherein the amino acid positions are relative to the reference sequence corresponding to residues 12 to 346 of SEQ ID NO: 2674, or relative to the reference sequence corresponding to SEQ ID NO: 2674.

In some embodiments, the recombinant polynucleotide comprises a polynucleotide sequence encoding an engineered RNA ligase comprising an amino acid sequence comprising at least a substitution or substitution set at amino acid position(s) 208, 25, 90, 141, 258, 248, 318, 117, 299, 33, 80, 253, 83, 254, 256, 30, 327, 73, 100, 97, 119, 265, 296, 32, 156, 40, 105, 107, 41/114, 41, or 64, wherein the amino acid positions are relative to the reference sequence corresponding to residues 12 to 346 of SEQ ID NO: 2674, or relative to the reference sequence corresponding to SEQ ID NO: 2674.

In some embodiments, the recombinant polynucleotide comprises a polynucleotide sequence encoding an engineered RNA ligase comprising an amino acid sequence comprising at least a substitution or substitution set at amino acid position(s) 162, 208, 25, 103, 346, 42, 74, 113, 13, 228, 318, 121, 299, 33, 342, 253, 127, 254, 292, 30, 67, 86, 165, 173, 300, 317, 326, 11, 229, 97, 71, 17, 210, 241, 271, 40, 56, or 95, wherein the amino acid positions are relative to the reference sequence corresponding to residues 12 to 346 of SEQ ID NO: 2674, or relative to the reference sequence corresponding to SEQ ID NO: 2674.

In some embodiments, the recombinant polynucleotide comprises a polynucleotide sequence encoding an engineered RNA ligase comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12 to 346 of SEQ ID NO: 2754, or to the reference sequence corresponding to SEQ ID NO: 2754, wherein the amino acid sequence comprises one or more substitutions relative to the reference sequence corresponding to residues 12 to 346 of SEQ ID NO: 2754, or relative to the reference sequence corresponding to SEQ ID NO: 2754.

In some embodiments, the recombinant polynucleotide comprises a polynucleotide sequence encoding an engineered RNA ligase comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12 to 346 of an even-numbered SEQ ID NO. of SEQ ID NOs: 2784-2820, or to the reference sequence corresponding to an even-numbered SEQ ID NO. of SEQ ID NOs: 2784-2820, wherein the amino acid sequence comprises one or more substitutions relative to the reference sequence corresponding to residues 12 to 346 of SEQ ID NO: 2754, or relative to the reference sequence corresponding to SEQ ID NO: 2754.

In some embodiments, the recombinant polynucleotide comprises a polynucleotide sequence encoding an engineered RNA ligase comprising an amino acid sequence comprising at least a substitution or substitution set at amino acid position(s) 106/264/280, 280/330, 28/106/264/291, 28/264/280/330, 57/60/135/264/291, 28/135/264/280, 106/264/280/291, 28/106/264/280, 280/299, 28/57/89/106/264/280/291, 264, 185/291, 28/106/299, 28/214/264/299, 264/330, 28/89/214/291, 28/185/271/310, 122/271, or 89/106/135/264/280/330, wherein the amino acid positions are relative to the reference sequence corresponding to residues 12 to 346 of SEQ ID NO: 2754, or relative to the reference sequence corresponding to SEQ ID NO: 2754.

In some embodiments, the recombinant polynucleotide comprises a polynucleotide sequence encoding an engineered RNA ligase comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12 to 346 of SEQ ID NO: 2786, or to the reference sequence corresponding to SEQ ID NO: 2786, wherein the amino acid sequence comprises one or more substitutions relative to the reference sequence corresponding to residues 12 to 346 of SEQ ID NO: 2786, or relative to the reference sequence corresponding to SEQ ID NO: 2786.

In some embodiments, the recombinant polynucleotide comprises a polynucleotide sequence encoding an engineered RNA ligase comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12 to 346 of an even-numbered SEQ ID NO. of SEQ ID NOs: 3014-3058, or to the reference sequence corresponding to an even-numbered SEQ ID NO. of SEQ ID NOs: 3014-3058, wherein the amino acid sequence comprises one or more substitutions relative to the reference sequence corresponding to residues 12 to 346 of SEQ ID NO: 2786, or relative to the reference sequence corresponding to SEQ ID NO: 2786.

In some embodiments, the recombinant polynucleotide comprises a polynucleotide sequence encoding an engineered RNA ligase comprising an amino acid sequence comprising at least a substitution or substitution set at amino acid position(s) 25/228/248/264, 32/113/228/248, 25/32/113/241/248/264/299, 25/113/248/264/318, 25/127/228/264/299/318, 25/113/228/248/327, 228/241/264, 264/299/318/327, 25/113/241/248/264/318, 25/127/248/299/318, 113/127/228/264/299, 25/127, 25/228/248/264/327, 25/113/264/299, 32/113/264/318, 25/32/241/264/327, 113/264/327, 25/127/228/248/318, 327, 32/113/254/264, 25/228/241/264, 25/113/228/241/318, or 113/327, wherein the amino acid positions are relative to the reference sequence corresponding to residues 12 to 346 of SEQ ID NO: 2786, or relative to the reference sequence corresponding to SEQ ID NO: 2786.

In some embodiments, the recombinant polynucleotide comprises a polynucleotide sequence encoding an engineered RNA ligase comprising an amino acid sequence comprising a substitution at an amino acid position provided in Tables 6.1, 6.2, 7.1, 8.1, 8.2, 9.1, 10.1, 11.1, 12.1, 13.1, 14.1, 15.1, 16.1, 17.1, 18.1, 19.1, 20.1, 21.1, 22.1, 23.1, 24.1, 25.1, and 26.1, wherein the amino acid positions are relative to the reference sequence corresponding to residues 12 to 346 of SEQ ID NO: 54, 244, 648, 862, 956, 1088, 1416, 1612, 1840, 1866, 1954, 2062, 2674, 2754, or 2786, or relative to the reference sequence corresponding to SEQ ID NO: 54, 244, 648, 862, 956, 1088, 1416, 1612, 1840, 1866, 1954, 2062, 2674, 2754, or 2786.

In some embodiments, the recombinant polynucleotide comprises a polynucleotide sequence encoding an engineered RNA ligase comprising an amino acid sequence comprising at least one substitution provided in Tables 6.1, 6.2, 7.1, 8.1, 8.2, 9.1, 10.1, 11.1, 12.1, 13.1, 14.1, 15.1, 16.1, 17.1, 18.1, 19.1, 20.1, 21.1, 22.1, 23.1, 24.1, 25.1, and 26.1, wherein the amino acid positions are relative to the reference sequence corresponding to residues 12 to 346 of SEQ ID NO: 54, 244, 648, 862, 956, 1088, 1416, 1612, 1840, 1866, 1954, 2062, 2674, 2754, or 2786, or relative to the reference sequence corresponding to SEQ ID NO: 54, 244, 648, 862, 956, 1088, 1416, 1612, 1840, 1866, 1954, 2062, 2674, 2754, or 2786.

In some embodiments, the recombinant polynucleotide comprises a polynucleotide sequence encoding an engineered RNA ligase comprising at least a substitution or substitution set at amino acid position(s) provided in Tables 6.1, 6.2, 7.1, 8.1, 8.2, 9.1, 10.1, 11.1, 12.1, 13.1, 14.1, 15.1, 16.1, 17.1, 18.1, 19.1, 20.1, 21.1, 22.1, 23.1, 24.1, 25.1, and 26.1, wherein the amino acid positions are relative to the reference sequence corresponding to residues 12 to 346 of SEQ ID NO: 54, 244, 648, 862, 956, 1088, 1416, 1612, 1840, 1866, 1954, 2062, 2674, 2754, or 2786, or relative to the reference sequence corresponding to SEQ ID NO: 54, 244, 648, 862, 956, 1088, 1416, 1612, 1840, 1866, 1954, 2062, 2674, 2754, or 2786.

In some embodiments, the recombinant polynucleotide comprises a polynucleotide sequence encoding an engineered RNA ligase comprising at least a substitution or substitution set of an RNA ligase variant provided in Tables 6.1, 6.2, 7.1, 8.1, 8.2, 9.1, 10.1, 11.1, 12.1, 13.1, 14.1, 15.1, 16.1, 17.1, 18.1, 19.1, 20.1, 21.1, 22.1, 23.1, 24.1, 25.1, and 26.1, wherein the amino acid positions are relative to the reference sequence corresponding to residues 12 to 346 of SEQ ID NO: 54, 244, 648, 862, 956, 1088, 1416, 1612, 1840, 1866, 1954, 2062, 2674, 2754, or 2786, or relative to the reference sequence corresponding to SEQ ID NO: 54, 244, 648, 862, 956, 1088, 1416, 1612, 1840, 1866, 1954, 2062, 2674, 2754, or 2786.

In some embodiments, the recombinant polynucleotide comprises a polynucleotide sequence encoding an engineered RNA ligase comprising an amino acid sequence having least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to a reference sequence comprising a substitution or substitution set provided in Tables 6.1, 6.2, 7.1, 8.1, 8.2, 9.1, 10.1, 11.1, 12.1, 13.1, 14.1, 15.1, 16.1, 17.1, 18.1, 19.1, 20.1, 21.1, 22.1, 23.1, 24.1, 25.1, and 26.1, wherein the amino acid positions are relative to the reference sequence corresponding to residues 12 to 346 of SEQ ID NO: 54, 244, 648, 862, 956, 1088, 1416, 1612, 1840, 1866, 1954, 2062, 2674, 2754, or 2786, or relative to the reference sequence corresponding to SEQ ID NO: 54, 244, 648, 862, 956, 1088, 1416, 1612, 1840, 1866, 1954, 2062, 2674, 2754, or 2786.

In some embodiments, the recombinant polynucleotide comprises a polynucleotide sequence encoding an engineered RNA ligase comprising an amino acid sequence having least 70%, 75%, 80%, 81%, 82%, 83%, 84%, 85%, 86%, 87%, 88%, 89%, 90%, 91%, 92%, 93%, 94%, 95%, 96%, 97%, 98%, 99% or more sequence identity to the sequence comprising residues 12 to 346 of an engineered RNA ligase set forth in Tables 6.1, 6.2, 7.1, 8.1, 8.2, 9.1, 10.1, 11.1, 12.1, 13.1, 14.1, 15.1, 16.1, 17.1, 18.1, 19.1, 20.1, 21.1, 22.1, 23.1, 24.1, 25.1, and 26.1, or to the sequence of an engineered RNA ligase set forth in Tables 6.1, 6.2, 7.1, 8.1, 8.2, 9.1, 10.1, 11.1, 12.1, 13.1, 14.1, 15.1, 16.1, 17.1, 18.1, 19.1, 20.1, 21.1, 22.1, 23.1, 24.1, 25.1, and 26.1.

In some embodiments, the recombinant polynucleotide comprises a polynucleotide sequence encoding an engineered RNA ligase comprising residues 12 to 346 of an even numbered SEQ ID NO. of SEQ ID NOs: 4-1470 and 1478-3058, or an amino acid sequence comprising an even numbered SEQ ID NO. of SEQ ID NOs: 4-1470 and 1478-3058, optionally wherein the amino acid sequence has 1, 2, 3, 4, 5, 6, 7, 8, 9, or up to 10 substitutions.

In some embodiments, the recombinant polynucleotide comprises a polynucleotide sequence encoding an engineered RNA ligase comprising residues 12 to 346 of SEQ ID NO: 54, 244, 648, 862, 956, 1088, 1416, 1612, 1840, 1866, 1954, 2062, 2674, 2754, or 2786, or an amino acid sequence comprising SEQ ID NO: 54, 244, 648, 862, 956, 1088, 1416, 1612, 1840, 1866, 1954, 2062, 2674, 2754, or 2786, optionally wherein the amino acid sequence has 1, 2, 3, 4, 5, 6, 7, 8, 9, or up to 10 substitutions.

In some embodiments, the recombinant polynucleotide comprises a polynucleotide sequence having at least 70%, 75%, 80%, 81%, 82%, 83%, 84%, 85%, 90%, 91%, 92%, 93%, 94%, 95%, 96%, 97%, 98%, 99%, or more sequence identity to a reference polynucleotide sequence corresponding to nucleotide residues 34 to 1038 of an odd numbered SEQ ID NO. of SEQ ID NOs: 3-1469 and 1477-3057, or to a reference polynucleotide sequence corresponding an odd numbered SEQ ID NO. of SEQ ID NOs: 3-1469 and 1477-3057, wherein the recombinant polynucleotide encodes an RNA ligase.

In some embodiments, the recombinant polynucleotide comprises a polynucleotide sequence having at least 70%, 75%, 75%, 80%, 81%, 82%, 83%, 84%, 85%, 90%, 91%, 92%, 93%, 94%, 95%, 96%, 97%, 98%, 99%, or more sequence identity to a reference polynucleotide sequence corresponding to nucleotide residues 34 to 1038 of SEQ ID NO: 53, 243, 647, 861, 955, 1087, 1415, 1611, 1839, 1865, 1953, 2061, 2673, 2753, or 2785, or to a reference polynucleotide sequence corresponding to SEQ ID NO: 53, 243, 647, 861, 955, 1087, 1415, 1611, 1839, 1865, 1953, 2061, 2673, 2753, or 2785, wherein the recombinant polynucleotide encodes an RNA ligase.

In some embodiments, the recombinant polynucleotide comprises a polynucleotide sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to nucleotide residues 34 to 1038 of SEQ ID NO: 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 213, 215, 217, 219, 221, 223, 225, 227, 229, 231, 233, 235, 237, 239, 241, 243, 245, 247, 249, 251, 253, 255, 257, 259, 261, 263, 265, 267, 269, 271, 273, 275, 277, 279, 281, 283, 285, 287, 289, 291, 293, 295, 297, 299, 301, 303, 305, 307, 309, 311, 313, 315, 317, 319, 321, 323, 325, 327, 329, 331, 333, 335, 337, 339, 341, 343, 345, 347, 349, 351, 353, 355, 357, 359, 361, 363, 365, 367, 369, 371, 373, 375, 377, 379, 381, 383, 385, 387, 389, 391, 393, 395, 397, 399, 401, 403, 405, 407, 409, 411, 413, 415, 417, 419, 421, 423, 425, 427, 429, 431, 433, 435, 437, 439, 441, 443, 445, 447, 449, 451, 453, 455, 457, 459, 461, 463, 465, 467, 469, 471, 473, 475, 477, 479, 481, 483, 485, 487, 489, 491, 493, 495, 497, 499, 501, 503, 505, 507, 509, 511, 513, 515, 517, 519, 521, 523, 525, 527, 529, 531, 533, 535, 537, 539, 541, 543, 545, 547, 549, 551, 553, 555, 557, 559, 561, 563, 565, 567, 569, 571, 573, 575, 577, 579, 581, 583, 585, 587, 589, 591, 593, 595, 597, 599, 601, 603, 605, 607, 609, 611, 613, 615, 617, 619, 621, 623, 625, 627, 629, 631, 633, 635, 637, 639, 641, 643, 645, 647, 649, 651, 653, 655, 657, 659, 661, 663, 665, 667, 669, 671, 673, 675, 677, 679, 681, 683, 685, 687, 689, 691, 693, 695, 697, 699, 701, 703, 705, 707, 709, 711, 713, 715, 717, 719, 721, 723, 725, 727, 729, 731, 733, 735, 737, 739, 741, 743, 745, 747, 749, 751, 753, 755, 757, 759, 761, 763, 765, 767, 769, 771, 773, 775, 777, 779, 781, 783, 785, 787, 789, 791, 793, 795, 797, 799, 801, 803, 805, 807, 809, 811, 813, 815, 817, 819, 821, 823, 825, 827, 829, 831, 833, 835, 837, 839, 841, 843, 845, 847, 849, 851, 853, 855, 857, 859, 861, 863, 865, 867, 869, 871, 873, 875, 877, 879, 881, 883, 885, 887, 889, 891, 893, 895, 897, 899, 901, 903, 905, 907, 909, 911, 913, 915, 917, 919, 921, 923, 925, 927, 929, 931, 933, 935, 937, 939, 941, 943, 945, 947, 949, 951, 953, 955, 957, 959, 961, 963, 965, 967, 969, 971, 973, 975, 977, 979, 981, 983, 985, 987, 989, 991, 993, 995, 997, 999, 1001, 1003, 1005, 1007, 1009, 1011, 1013, 1015, 1017, 1019, 1021, 1023, 1025, 1027, 1029, 1031, 1033, 1035, 1037, 1039, 1041, 1043, 1045, 1047, 1049, 1051, 1053, 1055, 1057, 1059, 1061, 1063, 1065, 1067, 1069, 1071, 1073, 1075, 1077, 1079, 1081, 1083, 1085, 1087, 1089, 1091, 1093, 1095, 1097, 1099, 1101, 1103, 1105, 1107, 1109, 1111, 1113, 1115, 1117, 1119, 1121, 1123, 1125, 1127, 1129, 1131, 1133, 1135, 1137, 1139, 1141, 1143, 1145, 1147, 1149, 1151, 1153, 1155, 1157, 1159, 1161, 1163, 1165, 1167, 1169, 1171, 1173, 1175, 1177, 1179, 1181, 1183, 1185, 1187, 1189, 1191, 1193, 1195, 1197, 1199, 1201, 1203, 1205, 1207, 1209, 1211, 1213, 1215, 1217, 1219, 1221, 1223, 1225, 1227, 1229, 1231, 1233, 1235, 1237, 1239, 1241, 1243, 1245, 1247, 1249, 1251, 1253, 1255, 1257, 1259, 1261, 1263, 1265, 1267, 1269, 1271, 1273, 1275, 1277, 1279, 1281, 1283, 1285, 1287, 1289, 1291, 1293, 1295, 1297, 1299, 1301, 1303, 1305, 1307, 1309, 1311, 1313, 1315, 1317, 1319, 1321, 1323, 1325, 1327, 1329, 1331, 1333, 1335, 1337, 1339, 1341, 1343, 1345, 1347, 1349, 1351, 1353, 1355, 1357, 1359, 1361, 1363, 1365, 1367, 1369, 1371, 1373, 1375, 1377, 1379, 1381, 1383, 1385, 1387, 1389, 1391, 1393, 1395, 1397, 1399, 1401, 1403, 1405, 1407, 1409, 1411, 1413, 1415, 1417, 1419, 1421, 1423, 1425, 1427, 1429, 1431, 1433, 1435, 1437, 1439, 1441, 1443, 1445, 1447, 1449, 1451, 1453, 1455, 1457, 1459, 1461, 1463, 1465, 1467, 1469, 1477, 1479, 1481, 1483, 1485, 1487, 1489, 1491, 1493, 1495, 1497, 1499, 1501, 1503, 1505, 1507, 1509, 1511, 1513, 1515, 1517, 1519, 1521, 1523, 1525, 1527, 1529, 1531, 1533, 1535, 1537, 1539, 1541, 1543, 1545, 1547, 1549, 1551, 1553, 1555, 1557, 1559, 1561, 1563, 1565, 1567, 1569, 1571, 1573, 1575, 1577, 1579, 1581, 1583, 1585, 1587, 1589, 1591, 1593, 1595, 1597, 1599, 1601, 1603, 1605, 1607, 1609, 1611, 1613, 1615, 1617, 1619, 1621, 1623, 1625, 1627, 1629, 1631, 1633, 1635, 1637, 1639, 1641, 1643, 1645, 1647, 1649, 1651, 1653, 1655, 1657, 1659, 1661, 1663, 1665, 1667, 1669, 1671, 1673, 1675, 1677, 1679, 1681, 1683, 1685, 1687, 1689, 1691, 1693, 1695, 1697, 1699, 1701, 1703, 1705, 1707, 1709, 1711, 1713, 1715, 1717, 1719, 1721, 1723, 1725, 1727, 1729, 1731, 1733, 1735, 1737, 1739, 1741, 1743, 1745, 1747, 1749, 1751, 1753, 1755, 1757, 1759, 1761, 1763, 1765, 1767, 1769, 1771, 1773, 1775, 1777, 1779, 1781, 1783, 1785, 1787, 1789, 1791, 1793, 1795, 1797, 1799, 1801, 1803, 1805. 1807, 1809, 1811, 1812, 1813, 1815, 1817, 1819, 1821, 1823, 1825, 1827, 1829, 1831, 1833, 1835, 1837, 1839, 1841, 1843, 1845, 1847, 1849, 1851, 1853, 1855, 1857, 1859, 1861, 1863, 1865, 1867, 1869, 1871, 1873, 1875, 1877, 1879, 1881, 1883, 1885, 1887, 1889, 1891, 1893, 1895, 1897, 1899, 1901, 1903, 1905, 1907, 1909, 1911, 1913, 1915, 1917, 1919, 1921, 1923, 1925, 1927, 1929, 1931, 1933, 1935, 1937, 1939, 1941, 1943, 1945, 1947, 1949, 1951, 1953, 1955, 1957, 1959, 1961, 1963, 1965, 1967, 1969, 1971, 1973, 1975, 1977, 1979, 1981, 1983, 1985, 1987, 1989, 1991, 1993, 1995, 1997, 1999, 2001, 2003, 2005, 2007, 2009, 2011, 2013, 2015, 2017, 2019, 2021, 2023, 2025, 2027, 2029, 2031,2033, 2035, 2037, 2039, 2041, 2043, 2045, 2047, 2049, 2051, 2053, 2055, 2057, 2059, 2061, 2063, 2065, 2067, 2069, 2071, 2073, 2075, 2077, 2079, 2081, 2083, 2085, 2087, 2089, 2091, 2093, 2095, 2097, 2099, 2101, 2103, 2105, 2107, 2109, 2111, 2113, 2115, 2117, 2119, 2121, 2123, 2125, 2127, 2129, 2131, 2133, 2135, 2137, 2139, 2141, 2143, 2145, 2147, 2149, 2151, 2153, 2155, 2157, 2159, 2161, 2163, 2165, 2167, 2169, 2171, 2173, 2175, 2177, 2179, 2181, 2183, 2185, 2187, 2189, 2191, 2193, 2195, 2197, 2199, 2201, 2203, 2205, 2207, 2209, 2211, 2213, 2215, 2217, 2219, 2221, 2223, 2225, 2227, 2229, 2231, 2233, 2235, 2237, 2239, 2241, 2243, 2245, 2247, 2249, 2251, 2253, 2255, 2257, 2259, 2261, 2263, 2265, 2267, 2269, 2271, 2273, 2275, 2277, 2279, 2281, 2283, 2285, 2287, 2289, 2291, 2293, 2295, 2297, 2299, 2301, 2303, 2305, 2307, 2309, 2311, 2313, 2315, 2317, 2319, 2321, 2323, 2325, 2327, 2329, 2331, 2333, 2335, 2337, 2339, 2341, 2343, 2345, 2347, 2349, 2351, 2353, 2355, 2357, 2359, 2361, 2363, 2365, 2367, 2369, 2371, 2373, 2375, 2377, 2379, 2381, 2383, 2385, 2387, 2389, 2391, 2393, 2395, 2397, 2399, 2401, 2403, 2405, 2407, 2409, 2411, 2413, 2415, 2417, 2419, 2421, 2423, 2425, 2427, 2429, 2431, 2433, 2435, 2437, 2439, 2441, 2443, 2445, 2447, 2449, 2451, 2453, 2455, 2457, 2459, 2461, 2463, 2465, 2467, 2469, 2471, 2473, 2475, 2477, 2479, 2481, 2483, 2485, 2487, 2489, 2491, 2493, 2495, 2497, 2499, 2501, 2503, 2505, 2507, 2509, 2511, 2513, 2515, 2517, 2519, 2521, 2523, 2525, 2527, 2529, 2531, 2533, 2535, 2537, 2539, 2541, 2543, 2545, 2547, 2549, 2551, 2553, 2555, 2557, 2559, 2561, 2563, 2565, 2567, 2569, 2571, 2573, 2575, 2577, 2579, 2581, 2583, 2585, 2587, 2589, 2591, 2593, 2595, 2597, 2599, 2601, 2603, 2605, 2607, 2609, 2611, 2613, 2615, 2617, 2619, 2621, 2623, 2625, 2627, 2629, 2631, 2633, 2635, 2637, 2639, 2641, 2643, 2645, 2647, 2649, 2651, 2653, 2655, 2657, 2659, 2661, 2663, 2665, 2667, 2669, 2671, 2673, 2675, 2677, 2679, 2681, 2683, 2685, 2687, 2689, 2691, 2693, 2695, 2697, 2699, 2701, 2703, 2705, 2707, 2709, 2711, 2713, 2715, 2717, 2719, 2721, 2723, 2725, 2727, 2729, 2731, 2733, 2735, 2737, 2739, 2741, 2743, 2745, 2747, 2749, 2751, 2753, 2755, 2757, 2759, 2761, 2763, 2765, 2767, 2769, 2771, 2773, 2775, 2777, 2779, 2781, 2783, 2785, 2787, 2789, 2791, 2793, 2795, 2797, 2799, 2801, 2803, 2805, 2807, 2809, 2811, 2813, 2815, 2817, 2819, 2821, 2823, 2825, 2827, 2829, 2831, 2833, 2835, 2837, 2839, 2841, 2843, 2845, 2847, 2849, 2851, 2853, 2855, 2857, 2859, 2861, 2863, 2867, 2869, 2871, 2873, 2875, 2877, 2879, 2881, 2883, 2885, 2887, 2889, 2891, 2893, 2895, 2897, 2899, 2901, 2903, 2905, 2907, 2909, 2911, 2913, 2915, 2917, 2919, 2921, 2923, 2925, 2927, 2929, 2931, 2933, 2935, 2937, 2939, 2941, 2943, 2945, 2947, 2949, 2951, 2953, 2955, 2957, 2959, 2961, 2963, 2965, 2967, 2969, 2971, 2973, 2975, 2977, 2979, 2981, 2983, 2985, 2987, 2989, 2991, 2993, 2995, 2997, 2999, 3001, 3003, 3005, 3007, 3009, 3011, 3013, 3015, 3017, 3019, 3021, 3023, 3025, 3027, 3029, 3031, 3033, 3035, 3037, 3039, 3041, 3043, 3045, 3047, 3049, 3051, 3053, 3055, or 3057, wherein the recombinant polynucleotide encodes an RNA ligase.

In some embodiments, the recombinant polynucleotide comprises a polynucleotide sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99% or more sequence identity to the reference sequence corresponding to SEQ ID NO: 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 213, 215, 217, 219, 221, 223, 225, 227, 229, 231, 233, 235, 237, 239, 241, 243, 245, 247, 249, 251, 253, 255, 257, 259, 261, 263, 265, 267, 269, 271, 273, 275, 277, 279, 281, 283, 285, 287, 289, 291, 293, 295, 297, 299, 301, 303, 305, 307, 309, 311, 313, 315, 317, 319, 321, 323, 325, 327, 329, 331, 333, 335, 337, 339, 341, 343, 345, 347, 349, 351, 353, 355, 357, 359, 361, 363, 365, 367, 369, 371, 373, 375, 377, 379, 381, 383, 385, 387, 389, 391, 393, 395, 397, 399, 401, 403, 405, 407, 409, 411, 413, 415, 417, 419, 421, 423, 425, 427, 429, 431, 433, 435, 437, 439, 441, 443, 445, 447, 449, 451, 453, 455, 457, 459, 461, 463, 465, 467, 469, 471, 473, 475, 477, 479, 481, 483, 485, 487, 489, 491, 493, 495, 497, 499, 501, 503, 505, 507, 509, 511, 513, 515, 517, 519, 521, 523, 525, 527, 529, 531, 533, 535, 537, 539, 541, 543, 545, 547, 549, 551, 553, 555, 557, 559, 561, 563, 565, 567, 569, 571, 573, 575, 577, 579, 581, 583, 585, 587, 589, 591, 593, 595, 597, 599, 601, 603, 605, 607, 609, 611, 613, 615, 617, 619, 621, 623, 625, 627, 629, 631, 633, 635, 637, 639, 641, 643, 645, 647, 649, 651, 653, 655, 657, 659, 661, 663, 665, 667, 669, 671, 673, 675, 677, 679, 681, 683, 685, 687, 689, 691, 693, 695, 697, 699, 701, 703, 705, 707, 709, 711, 713, 715, 717, 719, 721, 723, 725, 727, 729, 731, 733, 735, 737, 739, 741, 743, 745, 747, 749, 751, 753, 755, 757, 759, 761, 763, 765, 767, 769, 771, 773, 775, 777, 779, 781, 783, 785, 787, 789, 791, 793, 795, 797, 799, 801, 803, 805, 807, 809, 811, 813, 815, 817, 819, 821, 823, 825, 827, 829, 831, 833, 835, 837, 839, 841, 843, 845, 847, 849, 851, 853, 855, 857, 859, 861, 863, 865, 867, 869, 871, 873, 875, 877, 879, 881, 883, 885, 887, 889, 891, 893, 895, 897, 899, 901, 903, 905, 907, 909, 911, 913, 915, 917, 919, 921, 923, 925, 927, 929, 931, 933, 935, 937, 939, 941, 943, 945, 947, 949, 951, 953, 955, 957, 959, 961, 963, 965, 967, 969, 971, 973, 975, 977, 979, 981, 983, 985, 987, 989, 991, 993, 995, 997, 999, 1001, 1003, 1005, 1007, 1009, 1011, 1013, 1015, 1017, 1019, 1021, 1023, 1025, 1027, 1029, 1031, 1033, 1035, 1037, 1039, 1041, 1043, 1045, 1047, 1049, 1051, 1053, 1055, 1057, 1059, 1061, 1063, 1065, 1067, 1069, 1071, 1073, 1075, 1077, 1079, 1081, 1083, 1085, 1087, 1089, 1091, 1093, 1095, 1097, 1099, 1101, 1103, 1105, 1107, 1109, 1111, 1113, 1115, 1117, 1119, 1121, 1123, 1125, 1127, 1129, 1131, 1133, 1135, 1137, 1139, 1141, 1143, 1145, 1147, 1149, 1151, 1153, 1155, 1157, 1159, 1161, 1163, 1165, 1167, 1169, 1171, 1173, 1175, 1177, 1179, 1181, 1183, 1185, 1187, 1189, 1191, 1193, 1195, 1197, 1199, 1201, 1203, 1205, 1207, 1209, 1211, 1213, 1215, 1217, 1219, 1221, 1223, 1225, 1227, 1229, 1231, 1233, 1235, 1237, 1239, 1241, 1243, 1245, 1247, 1249, 1251, 1253, 1255, 1257, 1259, 1261, 1263, 1265, 1267, 1269, 1271, 1273, 1275, 1277, 1279, 1281, 1283, 1285, 1287, 1289, 1291, 1293, 1295, 1297, 1299, 1301, 1303, 1305, 1307, 1309, 1311, 1313, 1315, 1317, 1319, 1321, 1323, 1325, 1327, 1329, 1331, 1333, 1335, 1337, 1339, 1341, 1343, 1345, 1347, 1349, 1351, 1353, 1355, 1357, 1359, 1361, 1363, 1365, 1367, 1369, 1371, 1373, 1375, 1377, 1379, 1381, 1383, 1385, 1387, 1389, 1391, 1393, 1395, 1397, 1399, 1401, 1403, 1405, 1407, 1409, 1411, 1413, 1415, 1417, 1419, 1421, 1423, 1425, 1427, 1429, 1431, 1433, 1435, 1437, 1439, 1441, 1443, 1445, 1447, 1449, 1451, 1453, 1455, 1457, 1459, 1461, 1463, 1465, 1467, 1469, 1477, 1479, 1481, 1483, 1485, 1487, 1489, 1491, 1493, 1495, 1497, 1499, 1501, 1503, 1505, 1507, 1509, 1511, 1513, 1515, 1517, 1519, 1521, 1523, 1525, 1527, 1529, 1531, 1533, 1535, 1537, 1539, 1541, 1543, 1545, 1547, 1549, 1551, 1553, 1555, 1557, 1559, 1561, 1563, 1565, 1567, 1569, 1571, 1573, 1575, 1577, 1579, 1581, 1583, 1585, 1587, 1589, 1591, 1593, 1595, 1597, 1599, 1601, 1603, 1605, 1607, 1609, 1611, 1613, 1615, 1617, 1619, 1621, 1623, 1625, 1627, 1629, 1631, 1633, 1635, 1637, 1639, 1641, 1643, 1645, 1647, 1649, 1651, 1653, 1655, 1657, 1659, 1661, 1663, 1665, 1667, 1669, 1671, 1673, 1675, 1677, 1679, 1681, 1683, 1685, 1687, 1689, 1691, 1693, 1695, 1697, 1699, 1701, 1703, 1705, 1707, 1709, 1711, 1713, 1715, 1717, 1719, 1721, 1723, 1725, 1727, 1729, 1731, 1733, 1735, 1737, 1739, 1741, 1743, 1745, 1747, 1749, 1751, 1753, 1755, 1757, 1759, 1761, 1763, 1765, 1767, 1769, 1771, 1773, 1775, 1777, 1779, 1781, 1783, 1785, 1787, 1789, 1791, 1793, 1795, 1797, 1799, 1801, 1803, 1805. 1807, 1809, 1811, 1812, 1813, 1815, 1817, 1819, 1821, 1823, 1825, 1827, 1829, 1831, 1833, 1835, 1837, 1839, 1841, 1843, 1845, 1847, 1849, 1851, 1853, 1855, 1857, 1859, 1861, 1863, 1865, 1867, 1869, 1871, 1873, 1875, 1877, 1879, 1881, 1883, 1885, 1887, 1889, 1891, 1893, 1895, 1897, 1899, 1901, 1903, 1905, 1907, 1909, 1911, 1913, 1915, 1917, 1919, 1921, 1923, 1925, 1927, 1929, 1931, 1933, 1935, 1937, 1939, 1941, 1943, 1945, 1947, 1949, 1951, 1953, 1955, 1957, 1959, 1961, 1963, 1965, 1967, 1969, 1971, 1973, 1975, 1977, 1979, 1981, 1983, 1985, 1987, 1989, 1991, 1993, 1995, 1997, 1999, 2001, 2003, 2005, 2007, 2009, 2011, 2013, 2015, 2017, 2019, 2021, 2023, 2025, 2027, 2029, 2031,2033, 2035, 2037, 2039, 2041, 2043, 2045, 2047, 2049, 2051, 2053, 2055, 2057, 2059, 2061, 2063, 2065, 2067, 2069, 2071, 2073, 2075, 2077, 2079, 2081, 2083, 2085, 2087, 2089, 2091, 2093, 2095, 2097, 2099, 2101, 2103, 2105, 2107, 2109, 2111, 2113, 2115, 2117, 2119, 2121, 2123, 2125, 2127, 2129, 2131, 2133, 2135, 2137, 2139, 2141, 2143, 2145, 2147, 2149, 2151, 2153, 2155, 2157, 2159, 2161, 2163, 2165, 2167, 2169, 2171, 2173, 2175, 2177, 2179, 2181, 2183, 2185, 2187, 2189, 2191, 2193, 2195, 2197, 2199, 2201, 2203, 2205, 2207, 2209, 2211, 2213, 2215, 2217, 2219, 2221, 2223, 2225, 2227, 2229, 2231, 2233, 2235, 2237, 2239, 2241, 2243, 2245, 2247, 2249, 2251, 2253, 2255, 2257, 2259, 2261, 2263, 2265, 2267, 2269, 2271, 2273, 2275, 2277, 2279, 2281, 2283, 2285, 2287, 2289, 2291, 2293, 2295, 2297, 2299, 2301, 2303, 2305, 2307, 2309, 2311, 2313, 2315, 2317, 2319, 2321, 2323, 2325, 2327, 2329, 2331, 2333, 2335, 2337, 2339, 2341, 2343, 2345, 2347, 2349, 2351, 2353, 2355, 2357, 2359, 2361, 2363, 2365, 2367, 2369, 2371, 2373, 2375, 2377, 2379, 2381, 2383, 2385, 2387, 2389, 2391, 2393, 2395, 2397, 2399, 2401, 2403, 2405, 2407, 2409, 2411, 2413, 2415, 2417, 2419, 2421, 2423, 2425, 2427, 2429, 2431, 2433, 2435, 2437, 2439, 2441, 2443, 2445, 2447, 2449, 2451, 2453, 2455, 2457, 2459, 2461, 2463, 2465, 2467, 2469, 2471, 2473, 2475, 2477, 2479, 2481, 2483, 2485, 2487, 2489, 2491, 2493, 2495, 2497, 2499, 2501, 2503, 2505, 2507, 2509, 2511, 2513, 2515, 2517, 2519, 2521, 2523, 2525, 2527, 2529, 2531, 2533, 2535, 2537, 2539, 2541, 2543, 2545, 2547, 2549, 2551, 2553, 2555, 2557, 2559, 2561, 2563, 2565, 2567, 2569, 2571, 2573, 2575, 2577, 2579, 2581, 2583, 2585, 2587, 2589, 2591, 2593, 2595, 2597, 2599, 2601, 2603, 2605, 2607, 2609, 2611, 2613, 2615, 2617, 2619, 2621, 2623, 2625, 2627, 2629, 2631, 2633, 2635, 2637, 2639, 2641, 2643, 2645, 2647, 2649, 2651, 2653, 2655, 2657, 2659, 2661, 2663, 2665, 2667, 2669, 2671, 2673, 2675, 2677, 2679, 2681, 2683, 2685, 2687, 2689, 2691, 2693, 2695, 2697, 2699, 2701, 2703, 2705, 2707, 2709, 2711, 2713, 2715, 2717, 2719, 2721, 2723, 2725, 2727, 2729, 2731, 2733, 2735, 2737, 2739, 2741, 2743, 2745, 2747, 2749, 2751, 2753, 2755, 2757, 2759, 2761, 2763, 2765, 2767, 2769, 2771, 2773, 2775, 2777, 2779, 2781, 2783, 2785, 2787, 2789, 2791, 2793, 2795, 2797, 2799, 2801, 2803, 2805, 2807, 2809, 2811, 2813, 2815, 2817, 2819, 2821, 2823, 2825, 2827, 2829, 2831, 2833, 2835, 2837, 2839, 2841, 2843, 2845, 2847, 2849, 2851, 2853, 2855, 2857, 2859, 2861, 2863, 2867, 2869, 2871, 2873, 2875, 2877, 2879, 2881, 2883, 2885, 2887, 2889, 2891, 2893, 2895, 2897, 2899, 2901, 2903, 2905, 2907, 2909, 2911, 2913, 2915, 2917, 2919, 2921, 2923, 2925, 2927, 2929, 2931, 2933, 2935, 2937, 2939, 2941, 2943, 2945, 2947, 2949, 2951, 2953, 2955, 2957, 2959, 2961, 2963, 2965, 2967, 2969, 2971, 2973, 2975, 2977, 2979, 2981, 2983, 2985, 2987, 2989, 2991, 2993, 2995, 2997, 2999, 3001, 3003, 3005, 3007, 3009, 3011, 3013, 3015, 3017, 3019, 3021, 3023, 3025, 3027, 3029, 3031, 3033, 3035, 3037, 3039, 3041, 3043, 3045, 3047, 3049, 3051, 3053, 3055, or 3057, wherein the recombinant polynucleotide encodes an RNA ligase.

In some embodiments, the recombinant polynucleotide comprises a polynucleotide sequence comprising nucleotide residues 34 to 1038 of an odd numbered SEQ ID NO. of SEQ ID NOs: 3-1469, or a polynucleotide sequence comprising an odd numbered SEQ ID NO. of SEQ ID NOs: 3-1469.

In some embodiments, the recombinant polynucleotide comprises a polynucleotide sequence comprising nucleotide residues 34 to 1038 of SEQ ID NO: 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 213, 215, 217, 219, 221, 223, 225, 227, 229, 231, 233, 235, 237, 239, 241, 243, 245, 247, 249, 251, 253, 255, 257, 259, 261, 263, 265, 267, 269, 271, 273, 275, 277, 279, 281, 283, 285, 287, 289, 291, 293, 295, 297, 299, 301, 303, 305, 307, 309, 311, 313, 315, 317, 319, 321, 323, 325, 327, 329, 331, 333, 335, 337, 339, 341, 343, 345, 347, 349, 351, 353, 355, 357, 359, 361, 363, 365, 367, 369, 371, 373, 375, 377, 379, 381, 383, 385, 387, 389, 391, 393, 395, 397, 399, 401, 403, 405, 407, 409, 411, 413, 415, 417, 419, 421, 423, 425, 427, 429, 431, 433, 435, 437, 439, 441, 443, 445, 447, 449, 451, 453, 455, 457, 459, 461, 463, 465, 467, 469, 471, 473, 475, 477, 479, 481, 483, 485, 487, 489, 491, 493, 495, 497, 499, 501, 503, 505, 507, 509, 511, 513, 515, 517, 519, 521, 523, 525, 527, 529, 531, 533, 535, 537, 539, 541, 543, 545, 547, 549, 551, 553, 555, 557, 559, 561, 563, 565, 567, 569, 571, 573, 575, 577, 579, 581, 583, 585, 587, 589, 591, 593, 595, 597, 599, 601, 603, 605, 607, 609, 611, 613, 615, 617, 619, 621, 623, 625, 627, 629, 631, 633, 635, 637, 639, 641, 643, 645, 647, 649, 651, 653, 655, 657, 659, 661, 663, 665, 667, 669, 671, 673, 675, 677, 679, 681, 683, 685, 687, 689, 691, 693, 695, 697, 699, 701, 703, 705, 707, 709, 711, 713, 715, 717, 719, 721, 723, 725, 727, 729, 731, 733, 735, 737, 739, 741, 743, 745, 747, 749, 751, 753, 755, 757, 759, 761, 763, 765, 767, 769, 771, 773, 775, 777, 779, 781, 783, 785, 787, 789, 791, 793, 795, 797, 799, 801, 803, 805, 807, 809, 811, 813, 815, 817, 819, 821, 823, 825, 827, 829, 831, 833, 835, 837, 839, 841, 843, 845, 847, 849, 851, 853, 855, 857, 859, 861, 863, 865, 867, 869, 871, 873, 875, 877, 879, 881, 883, 885, 887, 889, 891, 893, 895, 897, 899, 901, 903, 905, 907, 909, 911, 913, 915, 917, 919, 921, 923, 925, 927, 929, 931, 933, 935, 937, 939, 941, 943, 945, 947, 949, 951, 953, 955, 957, 959, 961, 963, 965, 967, 969, 971, 973, 975, 977, 979, 981, 983, 985, 987, 989, 991, 993, 995, 997, 999, 1001, 1003, 1005, 1007, 1009, 1011, 1013, 1015, 1017, 1019, 1021, 1023, 1025, 1027, 1029, 1031, 1033, 1035, 1037, 1039, 1041, 1043, 1045, 1047, 1049, 1051, 1053, 1055, 1057, 1059, 1061, 1063, 1065, 1067, 1069, 1071, 1073, 1075, 1077, 1079, 1081, 1083, 1085, 1087, 1089, 1091, 1093, 1095, 1097, 1099, 1101, 1103, 1105, 1107, 1109, 1111, 1113, 1115, 1117, 1119, 1121, 1123, 1125, 1127, 1129, 1131, 1133, 1135, 1137, 1139, 1141, 1143, 1145, 1147, 1149, 1151, 1153, 1155, 1157, 1159, 1161, 1163, 1165, 1167, 1169, 1171, 1173, 1175, 1177, 1179, 1181, 1183, 1185, 1187, 1189, 1191, 1193, 1195, 1197, 1199, 1201, 1203, 1205, 1207, 1209, 1211, 1213, 1215, 1217, 1219, 1221, 1223, 1225, 1227, 1229, 1231, 1233, 1235, 1237, 1239, 1241, 1243, 1245, 1247, 1249, 1251, 1253, 1255, 1257, 1259, 1261, 1263, 1265, 1267, 1269, 1271, 1273, 1275, 1277, 1279, 1281, 1283, 1285, 1287, 1289, 1291, 1293, 1295, 1297, 1299, 1301, 1303, 1305, 1307, 1309, 1311, 1313, 1315, 1317, 1319, 1321, 1323, 1325, 1327, 1329, 1331, 1333, 1335, 1337, 1339, 1341, 1343, 1345, 1347, 1349, 1351, 1353, 1355, 1357, 1359, 1361, 1363, 1365, 1367, 1369, 1371, 1373, 1375, 1377, 1379, 1381, 1383, 1385, 1387, 1389, 1391, 1393, 1395, 1397, 1399, 1401, 1403, 1405, 1407, 1409, 1411, 1413, 1415, 1417, 1419, 1421, 1423, 1425, 1427, 1429, 1431, 1433, 1435, 1437, 1439, 1441, 1443, 1445, 1447, 1449, 1451, 1453, 1455, 1457, 1459, 1461, 1463, 1465, 1467, 1469, 1477, 1479, 1481, 1483, 1485, 1487, 1489, 1491, 1493, 1495, 1497, 1499, 1501, 1503, 1505, 1507, 1509, 1511, 1513, 1515, 1517, 1519, 1521, 1523, 1525, 1527, 1529, 1531, 1533, 1535, 1537, 1539, 1541, 1543, 1545, 1547, 1549, 1551, 1553, 1555, 1557, 1559, 1561, 1563, 1565, 1567, 1569, 1571, 1573, 1575, 1577, 1579, 1581, 1583, 1585, 1587, 1589, 1591, 1593, 1595, 1597, 1599, 1601, 1603, 1605, 1607, 1609, 1611, 1613, 1615, 1617, 1619, 1621, 1623, 1625, 1627, 1629, 1631, 1633, 1635, 1637, 1639, 1641, 1643, 1645, 1647, 1649, 1651, 1653, 1655, 1657, 1659, 1661, 1663, 1665, 1667, 1669, 1671, 1673, 1675, 1677, 1679, 1681, 1683, 1685, 1687, 1689, 1691, 1693, 1695, 1697, 1699, 1701, 1703, 1705, 1707, 1709, 1711, 1713, 1715, 1717, 1719, 1721, 1723, 1725, 1727, 1729, 1731, 1733, 1735, 1737, 1739, 1741, 1743, 1745, 1747, 1749, 1751, 1753, 1755, 1757, 1759, 1761, 1763, 1765, 1767, 1769, 1771, 1773, 1775, 1777, 1779, 1781, 1783, 1785, 1787, 1789, 1791, 1793, 1795, 1797, 1799, 1801, 1803, 1805. 1807, 1809, 1811, 1812, 1813, 1815, 1817, 1819, 1821, 1823, 1825, 1827, 1829, 1831, 1833, 1835, 1837, 1839, 1841, 1843, 1845, 1847, 1849, 1851, 1853, 1855, 1857, 1859, 1861, 1863, 1865, 1867, 1869, 1871, 1873, 1875, 1877, 1879, 1881, 1883, 1885, 1887, 1889, 1891, 1893, 1895, 1897, 1899, 1901, 1903, 1905, 1907, 1909, 1911, 1913, 1915, 1917, 1919, 1921, 1923, 1925, 1927, 1929, 1931, 1933, 1935, 1937, 1939, 1941, 1943, 1945, 1947, 1949, 1951, 1953, 1955, 1957, 1959, 1961, 1963, 1965, 1967, 1969, 1971, 1973, 1975, 1977, 1979, 1981, 1983, 1985, 1987, 1989, 1991, 1993, 1995, 1997, 1999, 2001, 2003, 2005, 2007, 2009, 2011, 2013, 2015, 2017, 2019, 2021, 2023, 2025, 2027, 2029, 2031,2033, 2035, 2037, 2039, 2041, 2043, 2045, 2047, 2049, 2051, 2053, 2055, 2057, 2059, 2061, 2063, 2065, 2067, 2069, 2071, 2073, 2075, 2077, 2079, 2081, 2083, 2085, 2087, 2089, 2091, 2093, 2095, 2097, 2099, 2101, 2103, 2105, 2107, 2109, 2111, 2113, 2115, 2117, 2119, 2121, 2123, 2125, 2127, 2129, 2131, 2133, 2135, 2137, 2139, 2141, 2143, 2145, 2147, 2149, 2151, 2153, 2155, 2157, 2159, 2161, 2163, 2165, 2167, 2169, 2171, 2173, 2175, 2177, 2179, 2181, 2183, 2185, 2187, 2189, 2191, 2193, 2195, 2197, 2199, 2201, 2203, 2205, 2207, 2209, 2211, 2213, 2215, 2217, 2219, 2221, 2223, 2225, 2227, 2229, 2231, 2233, 2235, 2237, 2239, 2241, 2243, 2245, 2247, 2249, 2251, 2253, 2255, 2257, 2259, 2261, 2263, 2265, 2267, 2269, 2271, 2273, 2275, 2277, 2279, 2281, 2283, 2285, 2287, 2289, 2291, 2293, 2295, 2297, 2299, 2301, 2303, 2305, 2307, 2309, 2311, 2313, 2315, 2317, 2319, 2321, 2323, 2325, 2327, 2329, 2331, 2333, 2335, 2337, 2339, 2341, 2343, 2345, 2347, 2349, 2351, 2353, 2355, 2357, 2359, 2361, 2363, 2365, 2367, 2369, 2371, 2373, 2375, 2377, 2379, 2381, 2383, 2385, 2387, 2389, 2391, 2393, 2395, 2397, 2399, 2401, 2403, 2405, 2407, 2409, 2411, 2413, 2415, 2417, 2419, 2421, 2423, 2425, 2427, 2429, 2431, 2433, 2435, 2437, 2439, 2441, 2443, 2445, 2447, 2449, 2451, 2453, 2455, 2457, 2459, 2461, 2463, 2465, 2467, 2469, 2471, 2473, 2475, 2477, 2479, 2481, 2483, 2485, 2487, 2489, 2491, 2493, 2495, 2497, 2499, 2501, 2503, 2505, 2507, 2509, 2511, 2513, 2515, 2517, 2519, 2521, 2523, 2525, 2527, 2529, 2531, 2533, 2535, 2537, 2539, 2541, 2543, 2545, 2547, 2549, 2551, 2553, 2555, 2557, 2559, 2561, 2563, 2565, 2567, 2569, 2571, 2573, 2575, 2577, 2579, 2581, 2583, 2585, 2587, 2589, 2591, 2593, 2595, 2597, 2599, 2601, 2603, 2605, 2607, 2609, 2611, 2613, 2615, 2617, 2619, 2621, 2623, 2625, 2627, 2629, 2631, 2633, 2635, 2637, 2639, 2641, 2643, 2645, 2647, 2649, 2651, 2653, 2655, 2657, 2659, 2661, 2663, 2665, 2667, 2669, 2671, 2673, 2675, 2677, 2679, 2681, 2683, 2685, 2687, 2689, 2691, 2693, 2695, 2697, 2699, 2701, 2703, 2705, 2707, 2709, 2711, 2713, 2715, 2717, 2719, 2721, 2723, 2725, 2727, 2729, 2731, 2733, 2735, 2737, 2739, 2741, 2743, 2745, 2747, 2749, 2751, 2753, 2755, 2757, 2759, 2761, 2763, 2765, 2767, 2769, 2771, 2773, 2775, 2777, 2779, 2781, 2783, 2785, 2787, 2789, 2791, 2793, 2795, 2797, 2799, 2801, 2803, 2805, 2807, 2809, 2811, 2813, 2815, 2817, 2819, 2821, 2823, 2825, 2827, 2829, 2831, 2833, 2835, 2837, 2839, 2841, 2843, 2845, 2847, 2849, 2851, 2853, 2855, 2857, 2859, 2861, 2863, 2867, 2869, 2871, 2873, 2875, 2877, 2879, 2881, 2883, 2885, 2887, 2889, 2891, 2893, 2895, 2897, 2899, 2901, 2903, 2905, 2907, 2909, 2911, 2913, 2915, 2917, 2919, 2921, 2923, 2925, 2927, 2929, 2931, 2933, 2935, 2937, 2939, 2941, 2943, 2945, 2947, 2949, 2951, 2953, 2955, 2957, 2959, 2961, 2963, 2965, 2967, 2969, 2971, 2973, 2975, 2977, 2979, 2981, 2983, 2985, 2987, 2989, 2991, 2993, 2995, 2997, 2999, 3001, 3003, 3005, 3007, 3009, 3011, 3013, 3015, 3017, 3019, 3021, 3023, 3025, 3027, 3029, 3031, 3033, 3035, 3037, 3039, 3041, 3043, 3045, 3047, 3049, 3051, 3053, 3055, or 3057.

In some embodiments, the recombinant polynucleotide comprises a polynucleotide sequence comprising SEQ ID NO: 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 213, 215, 217, 219, 221, 223, 225, 227, 229, 231, 233, 235, 237, 239, 241, 243, 245, 247, 249, 251, 253, 255, 257, 259, 261, 263, 265, 267, 269, 271, 273, 275, 277, 279, 281, 283, 285, 287, 289, 291, 293, 295, 297, 299, 301, 303, 305, 307, 309, 311, 313, 315, 317, 319, 321, 323, 325, 327, 329, 331, 333, 335, 337, 339, 341, 343, 345, 347, 349, 351, 353, 355, 357, 359, 361, 363, 365, 367, 369, 371, 373, 375, 377, 379, 381, 383, 385, 387, 389, 391, 393, 395, 397, 399, 401, 403, 405, 407, 409, 411, 413, 415, 417, 419, 421, 423, 425, 427, 429, 431, 433, 435, 437, 439, 441, 443, 445, 447, 449, 451, 453, 455, 457, 459, 461, 463, 465, 467, 469, 471, 473, 475, 477, 479, 481, 483, 485, 487, 489, 491, 493, 495, 497, 499, 501, 503, 505, 507, 509, 511, 513, 515, 517, 519, 521, 523, 525, 527, 529, 531, 533, 535, 537, 539, 541, 543, 545, 547, 549, 551, 553, 555, 557, 559, 561, 563, 565, 567, 569, 571, 573, 575, 577, 579, 581, 583, 585, 587, 589, 591, 593, 595, 597, 599, 601, 603, 605, 607, 609, 611, 613, 615, 617, 619, 621, 623, 625, 627, 629, 631, 633, 635, 637, 639, 641, 643, 645, 647, 649, 651, 653, 655, 657, 659, 661, 663, 665, 667, 669, 671, 673, 675, 677, 679, 681, 683, 685, 687, 689, 691, 693, 695, 697, 699, 701, 703, 705, 707, 709, 711, 713, 715, 717, 719, 721, 723, 725, 727, 729, 731, 733, 735, 737, 739, 741, 743, 745, 747, 749, 751, 753, 755, 757, 759, 761, 763, 765, 767, 769, 771, 773, 775, 777, 779, 781, 783, 785, 787, 789, 791, 793, 795, 797, 799, 801, 803, 805, 807, 809, 811, 813, 815, 817, 819, 821, 823, 825, 827, 829, 831, 833, 835, 837, 839, 841, 843, 845, 847, 849, 851, 853, 855, 857, 859, 861, 863, 865, 867, 869, 871, 873, 875, 877, 879, 881, 883, 885, 887, 889, 891, 893, 895, 897, 899, 901, 903, 905, 907, 909, 911, 913, 915, 917, 919, 921, 923, 925, 927, 929, 931, 933, 935, 937, 939, 941, 943, 945, 947, 949, 951, 953, 955, 957, 959, 961, 963, 965, 967, 969, 971, 973, 975, 977, 979, 981, 983, 985, 987, 989, 991, 993, 995, 997, 999, 1001, 1003, 1005, 1007, 1009, 1011, 1013, 1015, 1017, 1019, 1021, 1023, 1025, 1027, 1029, 1031, 1033, 1035, 1037, 1039, 1041, 1043, 1045, 1047, 1049, 1051, 1053, 1055, 1057, 1059, 1061, 1063, 1065, 1067, 1069, 1071, 1073, 1075, 1077, 1079, 1081, 1083, 1085, 1087, 1089, 1091, 1093, 1095, 1097, 1099, 1101, 1103, 1105, 1107, 1109, 1111, 1113, 1115, 1117, 1119, 1121, 1123, 1125, 1127, 1129, 1131, 1133, 1135, 1137, 1139, 1141, 1143, 1145, 1147, 1149, 1151, 1153, 1155, 1157, 1159, 1161, 1163, 1165, 1167, 1169, 1171, 1173, 1175, 1177, 1179, 1181, 1183, 1185, 1187, 1189, 1191, 1193, 1195, 1197, 1199, 1201, 1203, 1205, 1207, 1209, 1211, 1213, 1215, 1217, 1219, 1221, 1223, 1225, 1227, 1229, 1231, 1233, 1235, 1237, 1239, 1241, 1243, 1245, 1247, 1249, 1251, 1253, 1255, 1257, 1259, 1261, 1263, 1265, 1267, 1269, 1271, 1273, 1275, 1277, 1279, 1281, 1283, 1285, 1287, 1289, 1291, 1293, 1295, 1297, 1299, 1301, 1303, 1305, 1307, 1309, 1311, 1313, 1315, 1317, 1319, 1321, 1323, 1325, 1327, 1329, 1331, 1333, 1335, 1337, 1339, 1341, 1343, 1345, 1347, 1349, 1351, 1353, 1355, 1357, 1359, 1361, 1363, 1365, 1367, 1369, 1371, 1373, 1375, 1377, 1379, 1381, 1383, 1385, 1387, 1389, 1391, 1393, 1395, 1397, 1399, 1401, 1403, 1405, 1407, 1409, 1411, 1413, 1415, 1417, 1419, 1421, 1423, 1425, 1427, 1429, 1431, 1433, 1435, 1437, 1439, 1441, 1443, 1445, 1447, 1449, 1451, 1453, 1455, 1457, 1459, 1461, 1463, 1465, 1467, 1469, 1477, 1479, 1481, 1483, 1485, 1487, 1489, 1491, 1493, 1495, 1497, 1499, 1501, 1503, 1505, 1507, 1509, 1511, 1513, 1515, 1517, 1519, 1521, 1523, 1525, 1527, 1529, 1531, 1533, 1535, 1537, 1539, 1541, 1543, 1545, 1547, 1549, 1551, 1553, 1555, 1557, 1559, 1561, 1563, 1565, 1567, 1569, 1571, 1573, 1575, 1577, 1579, 1581, 1583, 1585, 1587, 1589, 1591, 1593, 1595, 1597, 1599, 1601, 1603, 1605, 1607, 1609, 1611, 1613, 1615, 1617, 1619, 1621, 1623, 1625, 1627, 1629, 1631, 1633, 1635, 1637, 1639, 1641, 1643, 1645, 1647, 1649, 1651, 1653, 1655, 1657, 1659, 1661, 1663, 1665, 1667, 1669, 1671, 1673, 1675, 1677, 1679, 1681, 1683, 1685, 1687, 1689, 1691, 1693, 1695, 1697, 1699, 1701, 1703, 1705, 1707, 1709, 1711, 1713, 1715, 1717, 1719, 1721, 1723, 1725, 1727, 1729, 1731, 1733, 1735, 1737, 1739, 1741, 1743, 1745, 1747, 1749, 1751, 1753, 1755, 1757, 1759, 1761, 1763, 1765, 1767, 1769, 1771, 1773, 1775, 1777, 1779, 1781, 1783, 1785, 1787, 1789, 1791, 1793, 1795, 1797, 1799, 1801, 1803, 1805. 1807, 1809, 1811, 1812, 1813, 1815, 1817, 1819, 1821, 1823, 1825, 1827, 1829, 1831, 1833, 1835, 1837, 1839, 1841, 1843, 1845, 1847, 1849, 1851, 1853, 1855, 1857, 1859, 1861, 1863, 1865, 1867, 1869, 1871, 1873, 1875, 1877, 1879, 1881, 1883, 1885, 1887, 1889, 1891, 1893, 1895, 1897, 1899, 1901, 1903, 1905, 1907, 1909, 1911, 1913, 1915, 1917, 1919, 1921, 1923, 1925, 1927, 1929, 1931, 1933, 1935, 1937, 1939, 1941, 1943, 1945, 1947, 1949, 1951, 1953, 1955, 1957, 1959, 1961, 1963, 1965, 1967, 1969, 1971, 1973, 1975, 1977, 1979, 1981, 1983, 1985, 1987, 1989, 1991, 1993, 1995, 1997, 1999, 2001, 2003, 2005, 2007, 2009, 2011, 2013, 2015, 2017, 2019, 2021, 2023, 2025, 2027, 2029, 2031,2033, 2035, 2037, 2039, 2041, 2043, 2045, 2047, 2049, 2051, 2053, 2055, 2057, 2059, 2061, 2063, 2065, 2067, 2069, 2071, 2073, 2075, 2077, 2079, 2081, 2083, 2085, 2087, 2089, 2091, 2093, 2095, 2097, 2099, 2101, 2103, 2105, 2107, 2109, 2111, 2113, 2115, 2117, 2119, 2121, 2123, 2125, 2127, 2129, 2131, 2133, 2135, 2137, 2139, 2141, 2143, 2145, 2147, 2149, 2151, 2153, 2155, 2157, 2159, 2161, 2163, 2165, 2167, 2169, 2171, 2173, 2175, 2177, 2179, 2181, 2183, 2185, 2187, 2189, 2191, 2193, 2195, 2197, 2199, 2201, 2203, 2205, 2207, 2209, 2211, 2213, 2215, 2217, 2219, 2221, 2223, 2225, 2227, 2229, 2231, 2233, 2235, 2237, 2239, 2241, 2243, 2245, 2247, 2249, 2251, 2253, 2255, 2257, 2259, 2261, 2263, 2265, 2267, 2269, 2271, 2273, 2275, 2277, 2279, 2281, 2283, 2285, 2287, 2289, 2291, 2293, 2295, 2297, 2299, 2301, 2303, 2305, 2307, 2309, 2311, 2313, 2315, 2317, 2319, 2321, 2323, 2325, 2327, 2329, 2331, 2333, 2335, 2337, 2339, 2341, 2343, 2345, 2347, 2349, 2351, 2353, 2355, 2357, 2359, 2361, 2363, 2365, 2367, 2369, 2371, 2373, 2375, 2377, 2379, 2381, 2383, 2385, 2387, 2389, 2391, 2393, 2395, 2397, 2399, 2401, 2403, 2405, 2407, 2409, 2411, 2413, 2415, 2417, 2419, 2421, 2423, 2425, 2427, 2429, 2431, 2433, 2435, 2437, 2439, 2441, 2443, 2445, 2447, 2449, 2451, 2453, 2455, 2457, 2459, 2461, 2463, 2465, 2467, 2469, 2471, 2473, 2475, 2477, 2479, 2481, 2483, 2485, 2487, 2489, 2491, 2493, 2495, 2497, 2499, 2501, 2503, 2505, 2507, 2509, 2511, 2513, 2515, 2517, 2519, 2521, 2523, 2525, 2527, 2529, 2531, 2533, 2535, 2537, 2539, 2541, 2543, 2545, 2547, 2549, 2551, 2553, 2555, 2557, 2559, 2561, 2563, 2565, 2567, 2569, 2571, 2573, 2575, 2577, 2579, 2581, 2583, 2585, 2587, 2589, 2591, 2593, 2595, 2597, 2599, 2601, 2603, 2605, 2607, 2609, 2611, 2613, 2615, 2617, 2619, 2621, 2623, 2625, 2627, 2629, 2631, 2633, 2635, 2637, 2639, 2641, 2643, 2645, 2647, 2649, 2651, 2653, 2655, 2657, 2659, 2661, 2663, 2665, 2667, 2669, 2671, 2673, 2675, 2677, 2679, 2681, 2683, 2685, 2687, 2689, 2691, 2693, 2695, 2697, 2699, 2701, 2703, 2705, 2707, 2709, 2711, 2713, 2715, 2717, 2719, 2721, 2723, 2725, 2727, 2729, 2731, 2733, 2735, 2737, 2739, 2741, 2743, 2745, 2747, 2749, 2751, 2753, 2755, 2757, 2759, 2761, 2763, 2765, 2767, 2769, 2771, 2773, 2775, 2777, 2779, 2781, 2783, 2785, 2787, 2789, 2791, 2793, 2795, 2797, 2799, 2801, 2803, 2805, 2807, 2809, 2811, 2813, 2815, 2817, 2819, 2821, 2823, 2825, 2827, 2829, 2831, 2833, 2835, 2837, 2839, 2841, 2843, 2845, 2847, 2849, 2851, 2853, 2855, 2857, 2859, 2861, 2863, 2867, 2869, 2871, 2873, 2875, 2877, 2879, 2881, 2883, 2885, 2887, 2889, 2891, 2893, 2895, 2897, 2899, 2901, 2903, 2905, 2907, 2909, 2911, 2913, 2915, 2917, 2919, 2921, 2923, 2925, 2927, 2929, 2931, 2933, 2935, 2937, 2939, 2941, 2943, 2945, 2947, 2949, 2951, 2953, 2955, 2957, 2959, 2961, 2963, 2965, 2967, 2969, 2971, 2973, 2975, 2977, 2979, 2981, 2983, 2985, 2987, 2989, 2991, 2993, 2995, 2997, 2999, 3001, 3003, 3005, 3007, 3009, 3011, 3013, 3015, 3017, 3019, 3021, 3023, 3025, 3027, 3029, 3031, 3033, 3035, 3037, 3039, 3041, 3043, 3045, 3047, 3049, 3051, 3053, 3055, or 3057.

In some embodiments, the recombinant polynucleotide comprises a polynucleotide sequence encoding an engineered RNA ligase comprising an amino acid sequence comprising
(a) one or more amino acid differences in the N-terminal domain at amino acid positions 12, 13, 15, 17, 18, 21, 23, 25, 26, 28, 29, 30, 32, 33, 37, 38, 39, 40, 41, 42, 45, 52, 54, 56, 57, 58, 60, 61, 62, 64, 65, 67, 71, 73, 74, 75, 77, 80, 81, 83, 84, 86, 87, 89, 90, 91, 93, 95, 97, 99, 100, 101, 102, 103, 104, 105, 106, 107, 109, 113, 114, 116, 117, 119, 121, 122, 123, 125, 126, 127, 128, 129, 131, 135, 137, 140, 141, 143, 144, 148, 150, 154, 156, 157, 158, 159, 160, 161, 162, 165, 169, 170, 171, 173, 175, 177, 178, 179, 181, 183, 184, 185, 190, 191, 192, 193, 194, 197, 198, 204, 206, 208, 210, 212, 213, 214, 215, 218, 223, 224, 226, 228, 229, 230, 232, 236, 238, 241, and 243, or at equivalent positions thereof;
(b) one or more amino acid differences in the C-terminal domain at amino acid positions 256, 257, 258, 260, 261, 264, 265, 266, 267, 268, 270, 271, 272, 274, 276, 277, 280, 282, 283, 286, 288, 289, 290, 291, 292, 294, 295, 296, 297, 298, 299, 300, 301, 302, 305, 306, 307, 310, 311, 315, 316, 317, 318, 323, 324, 325, 326, 327, 330, 331, 332, 334, 335, 336, 337, 338, 341, 342, 344, 345, and 346, or at equivalent positions thereof; and/or
(c) one or more amino acid differences in the linker region connecting the N-terminal and C-terminal domains at one or more amino acid positions 247, 248, 249, 250, 251, 252, 253, 254, and 255, or at equivalent positions thereof,
wherein the amino acid positions are relative to the reference sequence corresponding to residues 12-346 of SEQ ID NO: 3060.

In some embodiments, the recombinant polynucleotide comprises a polynucleotide sequence encoding an engineered RNA ligase comprising an amino acid sequence comprising
(a) in the N-terminal domain one or more amino acid residues 12A/F/L/M/P/R/T/V, 13W, 15M, 17P, 18A/H, 21S, 23K/T, 25A/D/F/G/L/M/S/T, 26L, 28A/L/M/V, 29D/S, 30A/L/T/V/W, 32E, 33A/I/L/T/V, 37A/F/K/L/M/R, 38A/E/L/R/S/T/V, 39P, 40D/I/P/S, 41E/I, 42T, 45S, 52C, 54L, 56I/L, 57V, 58M, 60A/C/D/R/S/V, 61A, 62A/V, 64A/Q/W, 65C, 67R, 71T, 73Q/W, 74D, 75A/D, 77M, 80E, 81Y, 83A/E/G/L/T/V,84V, 86D/G/L, 87E, 89C/D/E/G/L/M/N/Q/R/S/T/V/W/Y,90E/F,91A/E/V, 93K/V, 95A/C/G/I, 97A/E, 991, 100A/G/H/L/R/S/T/V, 101P/V/W, 102T, 103G/V, 104T, 105K/L/R, 106G/L/M/Q/R/V/W, 107M/S, 109F/L, 113L/M, 114E, 116L, 117A/Q, 119L, 121N, 122C/F/G/L/Q/T/W, 123K/R, 125K/T, 126M, 127P, 128E, 129L/W, 131T, 135A/H/M/Q/V, 137N/R, 140I, 141D/G, 143A/C/E/F/H/M/S/T/V, 144L, 148F, 150D/L, 154S/V, 156G/H/Q/S, 157F/L/Q/V, 158R/S, 159F, 160R, 161M/V, 162C, 165M, 169G/N, 170H/Y, 171S, 173M, 175A, 177K, 178V, 179E, 181E/N/P/R/S, 183L/V, 184L, 185C/F/G/K/M/P/S/T/V, 190I/M, 191C, 192D/E/F/L/N, 193K/Q, 194A/G/V, 197W, 198M, 204W, 206L, 208H/I/K/L/P/T, 210G/V, 212E/G/I/K/L, 213D/N/R/T, 214A/C/H/K/L/Q/Y, 215G/T, 218F, 223C, 224A/Q/R, 226R, 228D/L, 229W, 230T/V, 232G/M/N/R/S, 236L/V, 238K/S/V, 241E/G/Q/R, and 243H, or at equivalent positions thereof;
(b) in the C-terminal domain one or more amino acid residues 256A/E/G/M/S/W, 257D/E/G, 258D/M/P/V, 260G, 261D/P/T, 264A/E/G/S/V, 265A/F, 266C/I/L, 267I/M, 268F/G/M, 270L/M/V, 271A/E/G/T, 272M/S, 274A, 276A/G/M/V, 277A, 280L/N/W, 282A/M, 283C/V, 286D/E/F/M/V, 288E/L/R/W, 289A/D/E/I/P/T, 290D/H/N/P, 291H/L/Q/R/S/T/W, 292N/S, 294A/S/V/Y, 295C/F/G/K/L/M/P/Q/R/S/T/V/Y, 296E/G/L/Q/W, 297A/I/L, 298C/I/K/R/S/V, 299E/I/K/Q/R/T/V, 300A, 301A/F/I/L/M/V, 302A/C/I/L/S, 305V, 306G/L/T, 307K/R, 310H/L/M/N/Q/R/S/T, 311C, 315E/S, 316I/L/Y, 317E/F/G/Q/V/W, 318A/E/G/S, 323E/G, 324D/G/M, 325L, 326C/L, 327E/G/M/S, 330A/D/E/G/K/R, 331D/G/R/S/W, 332E/H/W, 334E/R, 335H/Q, 336I/L/M, 337A/V, 338G/L/M, 341A, 342R/S/T, 344K, 345Q/S, and 346S; or at equivalent positions thereof; and/or
(c) in the linker region connecting the N-terminal and C-terminal domains one or more amino acid residues 247I/L/R/S, 248F/G/I/K/T, 249K/L/R/S, 250E/I/S, 251C/E/F/G/L/R/S/T/Y, 252A/Q, 253L/W, 254E/K/L/N/Q/S/V, and 255N/S/V/Y, or at equivalent positions thereof,
wherein the amino acid positions are relative to the reference sequence corresponding to residues 12-346 of SEQ ID NO: 3060.

In some embodiments, the recombinant polynucleotide comprises a polynucleotide sequence encoding an engineered RNA ligase comprising in the amino acid sequence of the N-terminal domain one or more amino acid residues 12A/F/L/P/R/T/V, 23K/T, 25A/D/F/G/L/M/S/T, 29D/S, 37A/F/K/L/M/R, 38A/E/L/R/S/T/V, 75A/D, 86D/L, 89C/D/E/L/N/Q/R/S/T/V/Y,93K, 101P/V/W, 122C/F/G/L/Q/T/W, 143A/C/E/F/H/M/T/V, 157F/Q/V, 183L/V, 192E/F/L/N, 194A/G/V, 198M, 212E/G/I/K/L, 214A/C/H/K/L/Q/Y, 230T/V, and 241E/G/Q/R. or at equivalent positions thereof, wherein the amino acid positions are relative to the reference sequence corresponding to residues 12-346 of SEQ ID NO: 3060.

In some embodiments, the recombinant polynucleotide comprises a polynucleotide sequence encoding an engineered RNA ligase comprising in the amino acid sequence of the N-terminal domain one or more amino acid residues 12T, 23T, 25A, 29S, 37L, 38A/S, 75D, 86D, 89M, 93K, 101P, 122F, 157V, 183L, 192E, 194V, 198M, 212E, 214L, 226R, 230T/V, and 241G, or at equivalent positions thereof, wherein the amino acid positions are relative to the reference sequence corresponding to residues 12-346 of SEQ ID NO: 3060.

In some embodiments, the recombinant polynucleotide comprises a polynucleotide sequence encoding an engineered RNA ligase comprising in the amino acid sequence of the C-terminal domain one or more amino acid residues 266C/I/L, 268F/G/M, 280L/N/W, 291H/L/Q/R/S/T/W, 295C/F/K/L/M/P/Q/R/S/T/V/Y, 298C/I/K/R/S/V, 307K/R, 316I/L/Y, 318A/E/G/S, 326C/L, 330A/D/E/G/K/R, and 331D/G/R/S/W, or at equivalent positions thereof, wherein the amino acid positions are relative to the reference sequence corresponding to residues 12-346 of SEQ ID NO: 3060.

In some embodiments, the recombinant polynucleotide comprises a polynucleotide sequence encoding an engineered RNA ligase comprising in the amino acid sequence of the C-terminal domain one or more amino acid residues 266L, 268G, 280L, 291Q, 295V, 298S/V, 307R, 316L, 318S, 326L, 330A, and 331G, or at equivalent positions thereof, wherein the amino acid positions are relative to the reference sequence corresponding to residues 12-346 of SEQ ID NO: 3060.

In some embodiments, the recombinant polynucleotide comprises a polynucleotide sequence encoding an engineered RNA ligase comprising in the amino acid sequence of the linker region connecting the N-terminal and C-terminal domains one or more amino acid residues 248G/I/K/T, 249K/L/R/S, 250E/I/S, 251C/E/F/G/L/R/S/T/Y, and 254E/K/L/N/Q/S/V, or at equivalent positions thereof, wherein the amino acid positions are relative to the reference sequence corresponding to residues 12-346 of SEQ ID NO: 3060.

In some embodiments, the recombinant polynucleotide comprises a polynucleotide sequence encoding an engineered RNA ligase comprising in the amino acid sequence of the linker region connecting the N-terminal and C-terminal domains one or more amino acid residues 249L, 2501, 251E/S, and 254S, or at equivalent positions thereof, wherein the amino acid positions are relative to the reference sequence corresponding to residues 12-346 of SEQ ID NO: 3060.

In some embodiments, the recombinant polynucleotide comprises a polynucleotide sequence encoding an engineered RNA ligase comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99% or more sequence identity to the N-terminal domain corresponding to amino acid residues 12-246 of SEQ ID NO: 3060, wherein the amino acid sequence of the RNA ligase comprises one or more amino acid differences or amino acid residues in the N-terminal domain, as provided in the foregoing.

In some embodiments, the recombinant polynucleotide comprises a polynucleotide sequence encoding an engineered RNA ligase comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99% or more sequence identity to the C-terminal domain corresponding to amino acid residues 256-346 of SEQ ID NO: 3060, wherein the amino acid sequence of the RNA ligase comprises one or more amino acid differences or amino acid residues in the C-terminal domain, as provided in the foregoing.

In some embodiments, the recombinant polynucleotide comprises a polynucleotide sequence encoding an engineered RNA ligase comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99% or more sequence identity to the linker region corresponding to amino acid residues 247-255 of the reference sequence of SEQ ID NO: 3060, wherein the amino acid sequence of the RNA ligase comprises one or more amino acid differences or amino acid residues in the linker region, as provided in the foregoing.

In some embodiments, the present disclosure provides a recombinant polynucleotide capable of hybridizing under highly stringent conditions to a reference polynucleotide encoding an engineered RNA ligase polypeptide described herein, e.g., a recombinant polynucleotide provided in 5.1, 6.1, 6.2, 7.1, 8.1, 8.2, 9.1, 10.1, 11.1, 12.1, 13.1, 14.1, 15.1, 16.1, 17.1, 18.1, 19.1, 20.1, 21.1, 22.1, 23.1, 24.1, 25.1, and 26.1, or a reverse complement thereof. In some embodiments, the recombinant polynucleotide hybridizes under highly stringent conditions to a reference polynucleotide corresponding to nucleotide residues 34 to 1038 of SEQ ID NO: 53, 243, 647, 861, 955, 1087, 1415, 1611, 1839, 1865, 1953, 2061, 2673, 2753, or 2785, or to a reference polynucleotide sequence corresponding to SEQ ID NO: 53, 243, 647, 861, 955, 1087, 1415, 1611, 1839, 1865, 1953, 2061, 2673, 2753, or 2785, or a reverse complement thereof. In some embodiments, the recombinant polynucleotide hybridizes under highly stringent conditions to a polynucleotide sequence corresponding to nucleotide residues 34 to 1038 of an odd numbered SEQ ID NO. of SEQ ID NOs: 3-1469 and 1477-3057, or a polynucleotide sequence comprising an odd numbered SEQ ID NO. of SEQ ID NOs: 3-1469 and 1477-3057, or a reverse complement thereof.

In some embodiments, the present disclosure provides a recombinant polynucleotide capable of hybridizing under highly stringent conditions to a reverse complement of a reference polynucleotide encoding an engineered RNA ligase polypeptide described herein, wherein the recombinant polynucleotide hybridizing under stringent conditions encodes an RNA ligase polypeptide comprising an amino acid sequence having one or more amino acid differences as compared to SEQ ID NO: 2, 100, 244, 648, 862, 956, 1088, 1416, 1612, 1840, 1866, 1954, 2062, 2674, 2754, or 2786 at residue positions selected from any positions as set forth in Tables 5.1, 6.1, 6.2, 7.1, 8.1, 8.2, 9.1, 10.1, 11.1, 12.1, 13.1, 14.1, 15.1, 16.1, 17.1, 18.1, 19.1, 20.1, 21.1, 22.1, 23.1, 24.1, 25.1, and 26.1. In some embodiments, the recombinant polynucleotide that hybridizes under highly stringent conditions to a reverse complement of a reference polynucleotide encoding an engineered RNA ligase polypeptide described herein encodes an RNA ligase polypeptide having one or more amino acid differences present in an engineered RNA ligase having an amino acid sequence corresponding to residues 12 to 346 of an even numbered SEQ ID NO. of SEQ ID NOs: 4-1470 and 1478-3058, or an amino acid sequence comprising an even numbered SEQ ID NO. of SEQ ID NOs: 4-1470 and 1478-3058, wherein the amino acid differences are relative to SEQ ID NO: 2, 100, 244, 648, 862, 956, 1088, 1416, 1612, 1840, 1866, 1954, 2062, 2674, 2754, or 2786.

In some embodiments, the recombinant polynucleotide that hybridizes under highly stringent conditions comprises a polynucleotide sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99% or more sequence identity to a reference polynucleotide sequence corresponding to nucleotide residues 34 to 1038 of SEQ ID NO: 53, 243, 647, 861, 955, 1087, 1415, 1611, 1839, 1865, 1953, 2061, 2673, 2753, or 2785, or to a reference polynucleotide sequence corresponding to SEQ ID NO: 53, 243, 647, 861, 955, 1087, 1415, 1611, 1839, 1865, 1953, 2061, 2673, 2753, or 2785, or to a reference polynucleotide sequence corresponding to SEQ ID NO: 53, 243, 647, 861, 955, 1087, 1415, 1611, 1839, 1865, 1953, 2061, 2673, 2753, or 2785, or to a reference polynucleotide sequence corresponding to SEQ ID NO: 53, 243, 647, 861, 955, 1087, 1415, 1611, 1839, 1865, 1953, 2061, 2673, 2753, or 2785, or a reverse complement thereof. In some embodiments, the recombinant polynucleotide that hybridizes under highly stringent conditions comprises a polynucleotide sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99% or more sequence identity to a reference polynucleotide sequence corresponding to nucleotide residues 34 to 1038 of an odd-numbered SEQ ID NO. of SEQ ID NOs: 3-1469 and 1477-3057, or an odd-numbered SEQ ID NO. of SEQ ID NOs: 3-1469 and 1477-3057, or a reverse complement thereof.

In some additional embodiments, the polynucleotide hybridizing under highly stringent conditions comprises a polynucleotide sequence having at least 75%, 80%, 81%, 82%, 83%, 84%, 85%, 86%, 87%, 88%, 89%, 90%, 91%, 92%, 93%, 94%, 95%, 96%, 97%, 98%, 99% or more sequence identity to a reverse complement of a polynucleotide reference sequence corresponding to nucleotide residues 34 to 1038 of SEQ ID NO: 53, 243, 647, 861, 955, 1087, 1415, 1611, 1839, 1865, 1953, 2061, 2673, 2753, or 2785, or to a reference polynucleotide sequence corresponding to SEQ ID NO: 53, 243, 647, 861, 955, 1087, 1415, 1611, 1839, 1865, 1953, 2061, 2673, 2753, or 2785 encodes an engineered RNA ligase polypeptide. In some embodiments, the polynucleotide hybridizing under highly stringent conditions comprises a polynucleotide sequence having at least 75%, 80%, 81%, 82%, 83%, 84%, 85%, 86%, 87%, 88%, 89%, 90%, 91%, 92%, 93%, 94%, 95%, 96%, 97%, 98%, 99% or more sequence identity to a reverse complement of a polynucleotide reference sequence corresponding to nucleotide residues 34 to 1038 of an odd-numbered SEQ ID NO. of SEQ ID NOs: 3-1469 and 1477-3057, or an odd-numbered SEQ ID NO. of SEQ ID NOs: 3-1469 and 1477-3057 encodes an engineered RNA ligase polypeptide.

In some embodiments, a recombinant polynucleotide encoding any of the RNA ligases herein is manipulated in a variety of ways to facilitate expression of the RNA ligase polypeptide. In some embodiments, the recombinant polynucleotide encoding the RNA ligase comprises expression vectors where one or more control sequences is present to regulate the expression of the RNA ligase polynucleotides and/or polypeptides. Manipulation of the isolated polynucleotide prior to its insertion into a vector may be desirable or necessary depending on the expression vector utilized. Techniques for modifying polynucleotides and nucleic acid sequences utilizing recombinant DNA methods are well known in the art. In some embodiments, the control sequences include among others, promoters, leader sequences, polyadenylation sequences, propeptide sequences, signal peptide sequences, and transcription terminators.

In some embodiments, suitable promoters are selected based on the host cell selection. For bacterial host cells, suitable promoters for directing transcription of the nucleic acid constructs of the present disclosure, include, but are not limited to promoters obtained from the E. coli lac operon, Streptomyces coelicolor agarase gene (dagA), Bacillus subtilis levansucrase gene (sacB), Bacillus licheniformis alpha-amylase gene (amyL), Bacillus stearothermophilus maltogenic amylase gene (amyM), Bacillus amyloliquefaciens alpha-amylase gene (amyQ), Bacillus licheniformis penicillinase gene (penP), Bacillus subtilis xylA and xylB genes, and prokaryotic beta-lactamase gene (see, e.g., Villa-Kamaroff et al., Proc. Natl Acad. Sci. USA, 1978, 75:3727-3731), as well as the tac promoter (see, e.g., DeBoer et al., Proc. Natl Acad. Sci. USA, 1983, 80:21-25). Exemplary promoters for filamentous fungal host cells, include, but are not limited to promoters obtained from the genes for Aspergillus oryzae TAKA amylase, Rhizomucor miehei aspartic proteinase, Aspergillus niger neutral alpha-amylase, Aspergillus niger acid stable alpha-amylase, Aspergillus niger or Aspergillus awamori glucoamylase (glaA), Rhizomucor miehei lipase, Aspergillus oryzae alkaline protease, Aspergillus oryzae triose phosphate isomerase, Aspergillus nidulans acetamidase, and Fusarium oxysporum trypsin-like protease (see, e.g., WO 96/00787), as well as the NA2-tpi promoter (a hybrid of the promoters from the genes for Aspergillus niger neutral alpha-amylase and Aspergillus oryzae triose phosphate isomerase), and mutant, truncated, and hybrid promoters thereof. Exemplary yeast cell promoters can be from the genes for Saccharomyces cerevisiae enolase (ENO-1), Saccharomyces cerevisiae galactokinase (GAL1), Saccharomyces cerevisiae alcohol dehydrogenase/glyceraldehyde-3-phosphate dehydrogenase (ADH2/GAP), and Saccharomyces cerevisiae 3-phosphoglycerate kinase. Other useful promoters for yeast host cells are known in the art (see, e.g., Romanos et al., Yeast, 1992, 8:423-488). Exemplary promoters for use in insect cells include, but are not limited to, polyhedrin, p10, ELT, OpIE2, and hr5/ie1 promoters. Exemplary promoters for use in mammalian cells include, but are not limited to, those from cytomegalovirus (CMV), chicken β-actin promoter fused with the CMV enhancer, Simian vacuolating virus 40 (SV40), from Homo sapiens phosphoglycerate kinase, beta actin, elongation factor-la or glyceraldehyde-3-phosphate dehydrogenase, and from Gallus β-actin.

In some embodiments, the control sequence is a suitable transcription terminator sequence (i.e., a sequence recognized by a host cell to terminate transcription). In some embodiments, the terminator sequence is operably linked to the 3′ terminus of the nucleic acid sequence encoding the RNA ligase polypeptide. Any suitable terminator which is functional in the host cell of choice finds use in the present invention. For bacterial expression, the transcription terminators can be a Rho-dependent terminators that rely on a Rho transcription factor, or a Rho-independent, or intrinsic terminators, which do not require a transcription factor. Exemplary bacterial transcription terminators are described in Peters et al., J Mol Biol., 2011, 412 (5): 793-813. Exemplary transcription terminators for filamentous fungal host cells can be obtained from the genes for Aspergillus oryzae TAKA amylase, Aspergillus niger glucoamylase, Aspergillus nidulans anthranilate synthase, Aspergillus niger alpha-glucosidase, and Fusarium oxysporum trypsin-like protease. Exemplary terminators for yeast host cells can be obtained from the genes for Saccharomyces cerevisiae enolase, Saccharomyces cerevisiae cytochrome C (CYC1), and Saccharomyces cerevisiae glyceraldehyde-3-phosphate dehydrogenase. Other useful terminators for yeast host cells are known in the art (see, e.g., Romanos et al., Yeast, 1992, 8 (6): 423-88). Exemplary terminators for insect cells and mammalian cells include, but are not limited to, those from cytomegalovirus (CMV), Simian virus 40 (SV40), from Homo sapiens growth hormone hGH, from bovine growth hormone BGH, and from human or rabbit beta globulin.

In some embodiments, the control sequence is a suitable leader sequence, a non-translated region of an mRNA that is used for translation by the host cell. In some embodiments, the leader sequence is operably linked to the 5′ terminus of the nucleic acid sequence encoding the RNA ligase polypeptide. Any suitable leader sequence that is functional in the host cell of choice find use in the present invention. Exemplary leaders for filamentous fungal host cells are obtained from the genes for Aspergillus oryzae TAKA amylase, and Aspergillus nidulans triose phosphate isomerase. Suitable leaders for yeast host cells are obtained from the genes for Saccharomyces cerevisiae enolase (ENO-1), Saccharomyces cerevisiae 3-phosphoglycerate kinase, Saccharomyces cerevisiae alpha-factor, and Saccharomyces cerevisiae alcohol dehydrogenase/glyceraldehyde-3-phosphate dehydrogenase (ADH2/GAP). Suitable leaders for mammalian host cells include but are not limited to the 5′-UTR element present in orthopoxvirus mRNA.

In some embodiments, the control sequence is a polyadenylation sequence (i.e., a sequence operably linked to the 3′ terminus of the nucleic acid sequence and which, when transcribed, is recognized by the host cell as a signal to add polyadenosine residues to transcribed mRNA). Any suitable polyadenylation sequence which is functional in the host cell of choice finds use in the present invention. Exemplary polyadenylation sequences for filamentous fungal host cells include, but are not limited to the genes for Aspergillus oryzae TAKA amylase, Aspergillus niger glucoamylase, Aspergillus nidulans anthranilate synthase, Fusarium oxysporum trypsin-like protease, and Aspergillus niger alpha-glucosidase. Useful polyadenylation sequences for yeast host cells are known (see, e.g., Guo and Sherman, Mol. Cell. Biol., 1995, 15:5983-5990). Useful polyadenylation and 3′ UTR sequences for insect and mammalian host cells include, but are not limited to, OpIE2 polyA sequence, D. melanogaster metallothionein (Mt) polyA signal sequence, D. melanogaster alcohol dehydrogenase (adh), SV40 polyA signal sequence, and the 3′-UTRs of α- and β-globin mRNAs harboring sequence elements that increase the stability and translation of mRNA.

In some embodiments, the control sequence is also a signal peptide (i.e., a coding region that codes for an amino acid sequence linked to the amino terminus of a polypeptide and directs the encoded polypeptide into the cell's secretory pathway). In some embodiments, the 5′ end of the coding sequence of the nucleic acid sequence inherently contains a signal peptide coding region naturally linked in translation reading frame with the segment of the coding region that encodes the secreted polypeptide. Alternatively, in some embodiments, the 5′ end of the coding sequence contains a signal peptide coding region that is foreign to the coding sequence. Any suitable signal peptide coding region which directs the expressed polypeptide into the secretory pathway of a host cell of choice finds use for expression of the engineered polypeptide(s). Effective signal peptide coding regions for bacterial host cells are the signal peptide coding regions include, but are not limited to those obtained from the genes for Bacillus NCIB 11837 maltogenic amylase, Bacillus stearothermophilus alpha-amylase, Bacillus licheniformis subtilisin, Bacillus licheniformis beta-lactamase, Bacillus stearothermophilus neutral proteases (nprT, nprS, nprM), and Bacillus subtilis prsA. Further signal peptides are known in the art (see, e.g., Simonen and Palva, Microbiol. Rev., 1993, 57:109-137). In some embodiments, effective signal peptide coding regions for filamentous fungal host cells include, but are not limited to the signal peptide coding regions obtained from the genes for Aspergillus oryzae TAKA amylase, Aspergillus niger neutral amylase, Aspergillus niger glucoamylase, Rhizomucor miehei aspartic proteinase, Humicola insolens cellulase, and Humicola lanuginosa lipase. Useful signal peptides for yeast host cells include, but are not limited to those from the genes for Saccharomyces cerevisiae alpha-factor and Saccharomyces cerevisiae invertase. Useful signal peptides for insect and mammalian host cells include but are not limited to, those from the genes for immunoglobulin gamma (IgG) and the signal peptide in a human secreted protein, such as human beta-galactosidase polypeptide.

In some embodiments, the control sequence is a propeptide coding region that codes for an amino acid sequence positioned at the amino terminus of a polypeptide. The resultant polypeptide is referred to as a “proenzyme,” “propolypeptide,” or “zymogen.” A propolypeptide can be converted to a mature active polypeptide by catalytic or autocatalytic cleavage of the propeptide from the propolypeptide. The propeptide coding region may be obtained from any suitable source, including, but not limited to the genes for Bacillus subtilis alkaline protease (aprE), Bacillus subtilis neutral protease (nprT), Saccharomyces cerevisiae alpha-factor, Rhizomucor miehei aspartic proteinase, and Myceliophthora thermophila lactase (see, e.g., WO 95/33836). Where both signal peptide and propeptide regions are present at the amino terminus of a polypeptide, the propeptide region is positioned next to the amino terminus of a polypeptide and the signal peptide region is positioned next to the amino terminus of the propeptide region.

In some embodiments, regulatory sequences are also utilized. These sequences facilitate the regulation of the expression of the polypeptide relative to the growth of the host cell. Examples of regulatory systems are those that cause the expression of the gene to be turned on or off in response to a chemical or physical stimulus, including the presence of a regulatory compound. In prokaryotic host cells, suitable regulatory sequences include, but are not limited to the lac, tac, and trp operator systems. In yeast host cells, suitable regulatory systems include, but are not limited to the ADH2 system or GAL1 system. In filamentous fungi, suitable regulatory sequences include, but are not limited to the TAKA alpha-amylase promoter, Aspergillus niger glucoamylase promoter, and Aspergillus oryzae glucoamylase promoter. Exemplary inducible promoters regulated by exogenous agents include the zinc-inducible sheep metallothionine (MT) promoter, dexamethasone (Dex)-inducible promoter, mouse mammary tumor virus (MMTV) promoter; ecdysone insect promoter, tetracycline-inducible promoter system, RU486-inducible promoter system, and the rapamycin-inducible promoter system.

In another aspect, the present disclosure provides a recombinant expression vector comprising a recombinant polynucleotide encoding an engineered RNA ligase polypeptide, and one or more expression regulating regions such as a promoter and a terminator, a replication origin, etc., depending on the type of hosts into which they are to be introduced. In some embodiments, the various nucleic acid and control sequences described herein are joined together (i.e., operably linked) to produce recombinant expression vectors capable of expressing the encoded engineered RNA ligase polypeptide.

The recombinant expression vector may be any suitable vector (e.g., a plasmid or virus), that can be conveniently subjected to recombinant DNA procedures and bring about the expression of the RNA ligase polynucleotide. The choice of the vector typically depends on the compatibility of the vector with the host cell into which the vector is to be introduced. The vectors may be linear or closed circular plasmids.

In some embodiments, the expression vector is an autonomously replicating vector (i.e., a vector that exists as an extra-chromosomal entity, the replication of which is independent of chromosomal replication, such as a plasmid, an extra-chromosomal element, a minichromosome, or an artificial chromosome). The vector may contain any means for assuring self-replication. In some alternative embodiments, the vector is one in which, when introduced into the host cell, it is integrated into the genome and replicated together with the chromosome(s) into which it has been integrated. Furthermore, in some embodiments, a single vector or plasmid, or two or more vectors or plasmids which together contain the total DNA to be introduced into the genome of the host cell, and/or a transposon is utilized.

In some embodiment, the recombinant polynucleotides may be provided on a non-replicating expression vector or plasmid. In some embodiments, the non-replicating expression vector or plasmid can be based on viral vectors defective in replication (see, e.g., Travieso et al., npj Vaccines, 2022, Vol. 7, Article 75).

In some embodiments, the expression vector contains one or more selectable markers, which permit selection of transformed cells. A “selectable marker” is a gene, the product of which provides for biocide or viral resistance, resistance to heavy metals, prototrophy to auxotrophs, and the like. Examples of bacterial selectable markers include, but are not limited to the dal genes from Bacillus subtilis or Bacillus licheniformis, or markers, which confer antibiotic resistance such as ampicillin, kanamycin, chloramphenicol or tetracycline resistance. Suitable markers for yeast host cells include, but are not limited to ADE2, HIS3, LEU2, LYS2, MET3, TRP1, and URA3. Selectable markers for use in filamentous fungal host cells include, but are not limited to, amdS (acetamidase; e.g., from A. nidulans or A. orzyae), argB (ornithine carbamoyltransferases), bar (phosphinothricin acetyltransferase; e.g., from S. hygroscopicus), hph (hygromycin phosphotransferase), niaD (nitrate reductase), pyrG (orotidine-5′-phosphate decarboxylase; e.g., from A. nidulans or A. orzyae), sC (sulfate adenyltransferase), and trpC (anthranilate synthase), as well as equivalents thereof.

In another aspect, the present disclosure provides a host cell comprising a recombinant polynucleotide encoding an engineered RNA ligase polypeptide described herein, the polynucleotide(s) being operably linked to one or more control sequences for expression of the engineered RNA ligase enzyme(s) in the host cell. Host cells suitable for use in expressing the polypeptides encoded by the expression vectors of the present invention are known in the art and include but are not limited to, bacterial cells, such as E. coli, B. subtilis, Vibrio fluvialis, Streptomyces and Salmonella typhimurium cells; fungal cells, such as yeast cells (e.g., Saccharomyces cerevisiae or Pichia pastoris (ATCC Accession No. 201178)); insect cells such as Drosophila S2 and Spodoptera Sf9 cells; animal cells such as CHO, COS, BHK, 293, and Bowes melanoma cells; and plant cells. Exemplary host cells also include various Escherichia coli strains (e.g., W3110 (AfhuA) and BL21).

In another aspect, the present disclosure provides a method of producing the engineered RNA ligase polypeptides, the method comprising culturing a host cell capable of expressing a polynucleotide encoding the engineered RNA ligase polypeptide under conditions suitable for expression of the polypeptide such that the engineered RNA ligase is produced. In some embodiments, the method further comprises isolating the RNA ligase polypeptides from the culture and/or host cells. In some embodiments, the method further comprises purifying the expressed RNA ligase polypeptide, as described herein.

In some embodiments, the RNA ligase polypeptide expressed in a host cell is recovered from the cells and/or the culture medium using any one or more of the known techniques for protein purification, including, among others, lysozyme or detergent treatment, sonication, filtration, salting-out, ultra-centrifugation, and chromatography, such as described herein.

Chromatographic techniques for isolation/purification of the RNA ligase polypeptides include, among others, reverse phase chromatography, high-performance liquid chromatography, ion-exchange chromatography, hydrophobic-interaction chromatography, size-exclusion chromatography, gel electrophoresis, and affinity chromatography. Conditions for purifying the RNA ligase depends, in part, on factors such as net charge, hydrophobicity, hydrophilicity, molecular weight, molecular shape, etc., and will be apparent to those having skill in the art. In some embodiments, affinity techniques may be used to isolate the RNA ligase. For affinity chromatography purification, an antibody that specifically binds RNA ligase polypeptide may be used. In some embodiments, an affinity tag, e.g., His-tag, can be introduced into the RNA ligase polypeptide for purposes of isolation/purification.

It is contemplated that any suitable method for introducing polynucleotides for expression of the RNA ligase polypeptides into cells will find use in the present invention. Suitable techniques include, but are not limited to electroporation, biolistic particle bombardment, liposome mediated transfection, calcium chloride transfection, and protoplast fusion. In addition, the host cells are cultured using appropriate culture media and growth conditions known in the art for different host cell types.

In some embodiments, recombinant polypeptides (e.g., RNA ligase enzyme variants) can be produced using any suitable methods known the art. For example, there is a wide variety of different mutagenesis techniques known to those skilled in the art. In addition, mutagenesis kits are also available from many commercial molecular biology suppliers. Methods are available to make specific substitutions at defined amino acids (site-directed), specific or random mutations in a localized region of the gene (region-specific), or random mutagenesis over the entire gene (e.g., saturation mutagenesis). Numerous suitable methods are known to those in the art to generate enzyme variants, including but not limited to site-directed mutagenesis of single-stranded DNA or double-stranded DNA using PCR, cassette mutagenesis, gene synthesis, error-prone PCR, shuffling, and chemical saturation mutagenesis, or any other suitable method known in the art. Non-limiting examples of methods used for DNA and protein engineering are provided in the following patents: U.S. Pat. Nos. 6,117,679; 6,420,175; 6,376,246; 6,586,182; 7,747,391; 7,747,393; 7,783,428; and 8,383,346. After the variants are produced, they can be screened for any desired property (e.g., high or increased activity, or low or reduced activity, increased thermal activity, increased stability, increased substrate range, increased inhibitor resistance or tolerance, increased salt tolerance, and/or pH stability, etc.). Exemplary methods are provided in the Examples.

In some embodiments, the engineered RNA ligase polypeptides with the properties disclosed herein can be obtained by subjecting the polynucleotide encoding the naturally occurring or engineered RNA ligase polypeptide to a suitable mutagenesis and/or directed evolution methods known in the art, for example, as described herein. An exemplary directed evolution technique is mutagenesis and/or DNA shuffling (see, e.g., Stemmer, Proc. Natl. Acad. Sci. USA, 1994, 91:10747-10751; WO 95/22625; WO 97/0078; WO 97/35966; WO 98/27230; WO 00/42651; WO 01/75767 and U.S. Pat. No. 6,537,746). Other directed evolution procedures that can be used include, among others, staggered extension process (StEP), in vitro recombination (see, e.g., Zhao et al., Nat. Biotechnol., 1998, 16:258-261), mutagenic PCR (see, e.g., Caldwell et al., PCR Methods Appl., 1994, 3: S136-S140), and cassette mutagenesis (see, e.g., Black et al., Proc. Natl. Acad. Sci. USA, 1996, 93:3525-3529).

Mutagenesis and directed evolution methods can be applied to RNA ligase-encoding polynucleotides to generate variant libraries that can be expressed, screened, and assayed. Any suitable mutagenesis and directed evolution methods find use in the present disclosure and are known in the art (see, e.g., U.S. Pat. Nos. 5,605,793, 5,811,238, 5,830,721, 5,834,252, 5,837,458, 5,928,905, 6,096,548, 6,117,679, 6,132,970, 6,165,793, 6,180,406, 6,251,674, 6,265,201, 6,277,638, 6,287,861, 6,287,862, 6,291,242, 6,297,053, 6,303,344, 6,309,883, 6,319,713, 6,319,714, 6,323,030, 6,326,204, 6,335,160, 6,335,198, 6,344,356, 6,352,859, 6,355,484, 6,358,740, 6,358,742, 6,365,377, 6,365,408, 6,368,861, 6,372,497, 6,337,186, 6,376,246, 6,379,964, 6,387,702, 6,391,552, 6,391,640, 6,395,547, 6,406,855, 6,406,910, 6,413,745, 6,413,774, 6,420,175, 6,423,542, 6,426,224, 6,436,675, 6,444,468, 6,455,253, 6,479,652, 6,482,647, 6,483,011, 6,484,105, 6,489,146, 6,500,617, 6,500,639, 6,506,602, 6,506,603, 6,518,065, 6,519,065, 6,521,453, 6,528,311, 6,537,746, 6,573,098, 6,576,467, 6,579,678, 6,586,182, 6,602,986, 6,605,430, 6,613,514, 6,653,072, 6,686,515, 6,703,240, 6,716,631, 6,825,001, 6,902,922, 6,917,882, 6,946,296, 6,961,664, 6,995,017, 7,024,312, 7,058,515, 7,105,297, 7,148,054, 7,220,566, 7,288,375, 7,384,387, 7,421,347, 7,430,477, 7,462,469, 7,534,564, 7,620,500, 7,620,502, 7,629,170, 7,702,464, 7,747,391, 7,747,393, 7,751,986, 7,776,598, 7,783,428, 7,795,030, 7,853,410, 7,868,138, 7,783,428, 7,873,477, 7,873,499, 7,904,249, 7,957,912, 7,981,614, 8,014,961, 8,029,988, 8,048,674, 8,058,001, 8,076,138, 8,108,150, 8,170,806, 8,224,580, 8,377,681, 8,383,346, 8,457,903, 8,504,498, 8,589,085, 8,762,066, 8,768,871, 9,593,326, 9,665,694, 9,684,771, and all related PCT and non-US counterparts; Ling et al., Anal. Biochem., 1997, 254 (2): 157-78; Dale et al., Meth. Mol. Biol., 1996, 57:369-74; Smith, Ann. Rev. Genet., 1985, 19:423-462; Botstein et al., Science, 1985, 229:1193-1201; Carter, Biochem. J., 1986, 237:1-7; Kramer et al., Cell, 1984, 38:879-887; Wells et al., Gene, 1985, 34:315-323; Minshull et al., Curr. Op. Chem. Biol., 1999, 3:284-290; Christians et al., Nat. Biotechnol., 1999, 17:259-264; Crameri et al., Nature, 1998, 391:288-291; Crameri, et al., Nat. Biotechnol., 1997, 15:436-438; Zhang et al., Proc. Nat. Acad. Sci. U.S.A., 1997, 94:4504-4509; Crameri et al., Nat. Biotechnol., 1996, 14:315-319; Stemmer, Nature, 1994, 366:389-391; Stemmer, Proc. Nat. Acad. Sci. USA, 1994, 91:10747-10751; EP 3 049 973; WO 95/22625; WO 97/0078; WO 97/35966; WO 98/27230; WO 00/42651; WO 01/75767; WO 2009/152336; and WO 2015/048573, all of which are incorporated herein by reference).

In some embodiments, the clones obtained following mutagenesis treatment are screened by subjecting the enzyme preparations to a defined treatment conditions or assay conditions (e.g., temperature, pH, type of ligase substrate, input substrate concentration, nucleotide cofactors, etc.) and measuring enzyme activity after the treatments or other suitable assay conditions. Clones containing a polynucleotide encoding the polypeptide of interest are then isolated from the gene, sequenced to identify the nucleotide sequence changes (if any), and used to express the enzyme in a host cell. Measuring enzyme activity from the expression libraries can be performed using any suitable method known in the art and as described in the Examples.

In some embodiments, for engineered polypeptides of known sequence, the polynucleotides encoding the enzyme can be prepared by standard solid-phase methods, according to known synthetic methods. In some embodiments, fragments of up to about 100 bases can be individually synthesized, then joined (e.g., by enzymatic or chemical ligation methods, or polymerase mediated methods) to form any desired continuous sequence (see, e.g., Hughes et al., Cold Spring Harb Perspect Biol. 2017 January; 9 (1): a023812). For example, polynucleotides and oligonucleotides disclosed herein can be prepared by chemical synthesis using the classical phosphoramidite method (see, e.g., Beaucage et al., Tet. Lett., 1981, 22:1859-69; and Matthes et al., EMBO J., 1984, 3:801-05), as it is typically practiced in automated synthetic methods. According to the phosphoramidite method, oligonucleotides are synthesized (e.g., in an automatic DNA synthesizer), purified, annealed, ligated and cloned in appropriate vectors.

In some embodiments, a method for preparing the engineered RNA ligase polypeptide can comprise: (a) synthesizing a polynucleotide encoding a polypeptide comprising an amino acid sequence selected from the amino acid sequence of any variant as described herein, and (b) expressing the RNA ligase polypeptide encoded by the polynucleotide. In some embodiments of the method, the amino acid sequence encoded by the polynucleotide can optionally have one or several (e.g., up to 3, 4, 5, or up to 10) amino acid residue deletions, insertions and/or substitutions. In some embodiments, the amino acid sequence has optionally 1-2, 1-3, 1-4, 1-5, 1-6, 1-7, 1-8, 1-9, 1-10, 1-15, 1-20, 1-21, 1-22, 1-23, 1-24, 1-25, 1-30, 1-35, 1-40, 1-45, or 1-50 amino acid residue deletions, insertions and/or substitutions. In some embodiments, the amino acid sequence has optionally 1, 2, 3, 4, 5, 6, 7, 8, 9, 10, 11, 12, 13, 14, 15, 16, 17, 18, 19, 20, 21, 22, 23, 24, 25, 30, 30, 35, 40, 45, or 50 amino acid residue deletions, insertions and/or substitutions. In some embodiments, the amino acid sequence has optionally 1, 2, 3, 4, 5, 6, 7, 8, 9, 10, 11, 12, 13, 14, 15, 16, 18, 20, 21, 22, 23, 24, or 25 amino acid residue deletions, insertions and/or substitutions. In some embodiments, the substitutions are conservative or non-conservative substitutions.

### Composition of RNA Ligases

In a further aspect, the present disclosure provides compositions of the RNA ligases disclosed herein. In some embodiments, the composition comprises an engineered RNA ligase polypeptide described herein. In some embodiments, the engineered RNA ligase polypeptide in the compositions is isolated or purified. In some embodiments, the RNA ligase is combined with other components and compounds to provide compositions and formulations comprising the engineered RNA ligase polypeptide as appropriate for different applications and uses.

In some embodiments, the composition comprises at least one engineered RNA ligase described herein, e.g., an engineered RNA ligase provided in Tables 5.1, 6.1, 6.2, 7.1, 8.1, 8.2, 9.1, 10.1, 11.1, 12.1, 13.1, 14.1, 15.1, 16.1, 17.1, 18.1, 19.1, 20.1, 21.1, 22.1, 23.1, 24.1, 25.1, and 26.1, or in the Sequence Listing. In some embodiments, the composition comprises an engineered RNA ligase comprising an amino acid sequence comprising residues 12 to 346 of an even-numbered SEQ ID NO. of SEQ ID NOs: 4-1470 and 1478-3058, or an amino acid sequence comprising an even-numbered SEQ ID NO. of SEQ ID NOs: 4-1470 and 1478-3058.

In some embodiments, the composition further comprises one or more of a buffer, a nucleotide substrate (e.g., ATP or dATP), and/or at least one or more polynucleotide substrates, for example a polynucleotide substrate with modified nucleotides. In some embodiments, the composition comprises one or more of EDTA, DTT, and glycerol, such as for storage of the engineered RNA ligase.

In some embodiments, the composition further comprises an additive or ligation enhancing agent, including, among others, one or more of DMSO, betaine, polyethylene glycol (e.g., PEG 6000, PEG 8000, etc.), bovine serum albumin, Ficoll, and dextran (e.g., Dextran 6000). In some embodiments, the composition comprises 1% to 40% v/v of DMSO. In some embodiments, the composition comprises 0.1 M to 3 M betaine. In some embodiments, the composition comprises 0.5% to 20% w/v of PEG (e.g., PEG6000 or PEG8000).

In some embodiments, the composition further comprises an RNase inhibitor, including, among others, porcine RNase inhibitor, human placental RNase inhibitor, human liver RNase inhibitor, mouse RNase inhibitor, rat RNase inhibitor, and recombinantly expressed RNase inhibitors thereof.

In some embodiments, an engineered RNA ligase described herein is provided in solution, as a lyophilizate, or is immobilized on a substrate. In some embodiments, the substrate is a solid substrate, porous substrate, membrane, or particles. The enzyme can be entrapped in matrixes or membranes. In some embodiments, matrices include polymeric materials such as calcium-alginate, agar, k-carrageenin, polyacrylamide, agarose or derivatives thereof (e.g., cross-linked agarose), and collagen, or solid matrices, such as activated carbon, porous ceramic, and diatomaceous earth. In some embodiments, the matrix is a particle, a membrane, or a fiber. Types of membranes include, among others, nylon, cellulose, polysulfone, or polyacrylate.

In some embodiments, the enzyme is immobilized on the surface of a support material. In some embodiments, the enzyme is adsorbed on the support material. In some embodiments, the enzyme is immobilized on the support material by covalent attachment. Support materials include, among others, inorganic materials, such as alumina, silica, porous glass, ceramics, diatomaceous earth, clay, and bentonite, or organic materials, such as cellulose (CMC, DEAE-cellulose), starch, activated carbon, polyacrylate, polyacrylamide, polystyrene, and ion-exchange resins, such as Amberlite, Sephadex, and Dowex.

### Uses of Engineered RNA Ligase Polypeptides and Kits

In another aspect, the present disclosure provides uses of the engineered RNA ligases for polynucleotide synthesis, RNA repair, or other molecular biological uses.

In some embodiments, the engineered RNA ligase is used for ligating polynucleotides, including oligonucleotides. In some embodiments, the engineered RNA ligase is used for synthesizing polynucleotides from shorter oligonucleotides. In some embodiments, a method of ligating at least a first polynucleotide strand and a second polynucleotide strand comprises contacting a first polynucleotide strand and a second polynucleotide strand with an engineered RNA ligase described herein in presence of a nucleotide substrate under conditions suitable for ligation of the first polynucleotide strand to the second polynucleotide strand, wherein the first polynucleotide strand comprises a ligatable 5′-end and the second polynucleotide strand comprises a 3′-end ligatable to the 5′-end of the first polynucleotide strand.

In some embodiments, the second polynucleotide strand comprising a ligatable 3′-end has at least 2, 3, 4, 5 or 6 ribonucleotides at the 3′ terminal end. In some embodiments of the method, the 3′-end of the second polynucleotide strand is a 3′-OH. In some embodiments of the method, the 5′-end of the first polynucleotide strand is a 5′-phosphate. In some embodiments of the method, the internucleoside linkage contained in the first polynucleotide strand and/or second polynucleotide strand comprises a phosphate linkage.

In some embodiments, the first polynucleotide strand has at least a 5′-terminal nucleotide with a 2′-modified sugar moiety. In some embodiments, at least 2, 3, 4, or more of nucleotides at the 5′-terminal region of the first polynucleotide strand has 2′-modified sugar moiety. In some embodiments, the first polynucleotide strand has at least a 5′-terminal nucleotide with a non-natural internucleoside linkage to the adjacent 3′-nucleotide. In some embodiments, at least 2, 3, 4 or more nucleotides at the 5′-terminal region of the first polynucleotide strand has a non-natural internucleoside linkage. In some embodiments, the non-natural linkage is a phosphorothioate internucleoside linkage.

In some embodiments, the second polynucleotide strand has at least the 3′-terminal nucleotide with a 2′-modified sugar moiety. In some embodiments, at least 2, 3, 4, or more of nucleotides at the 3′-terminal region of the second polynucleotide strand have a 2′-modified sugar moiety. In some embodiments, the second polynucleotide strand has at least a 3′-terminal nucleotide with a non-natural internucleoside linkage to the adjacent 5′-nucleotide. In some embodiments, at least 2, 3, 4 or more nucleotides at the 3′-terminal region of the second polynucleotide strand has a non-natural internucleoside linkage. In some embodiments, the non-natural linkage is a phosphorothioate internucleoside linkage.

In some embodiments, the first polynucleotide strand has at least a 5′-terminal nucleotide with a 2′-modified sugar moiety, and the second polynucleotide strand has at least a 3′-terminal nucleotide with a 2′-modified sugar moiety. In some embodiments, at least 2, 3, 4, or more of nucleotides at the 5′-terminal region of the first polynucleotide strand have 2′-modified sugar moieties, and at least 2, 3, 4, or more of nucleotides at the 3′-terminal region of the second polynucleotide strand have 2′-modified sugar moieties. As further discussed below, in some embodiments, the 2′-modified sugar moiety is a 2′-O-methyl, 2′-O-ethyl, or 2′-fluoro. In some embodiments, the first polynucleotide strand has at least a 5′-terminal nucleotide with a non-natural internucleoside linkage to the adjacent 3′-nucleotide, and the second polynucleotide strand has at least a 3′-terminal nucleotide with a non-natural internucleoside linkage to the adjacent 5′-nucleotide. In some embodiments, at least 2, 3, 4 or more nucleotides at the 5′-terminal region of the first polynucleotide strand and at least 2, 3, 4 or more nucleotides at the 3′-terminal region of the second polynucleotide strand have a non-natural internucleoside linkage. In some embodiments, the non-natural internucleoside linkage is a phosphorothioate internucleoside linkage.

In some embodiments, the method further comprises a third polynucleotide strand, wherein the first polynucleotide strand and second polynucleotide strand hybridize adjacent to one another on the third polynucleotide strand to position the 5′-end of the first polynucleotide strand adjacent to the 3′-end of the second polynucleotide strand, e.g., to form a nick. In some embodiments of the method, the third polynucleotide strand is continuous with the first polynucleotide strand or second polynucleotide strand. In some embodiments of the method, the third polynucleotide strand is continuous with the first polynucleotide strand and second polynucleotide strand to form a single continuous polynucleotide ligase substrate. In some embodiments, the third polynucleotide strand is RNA, DNA, or a mixture of RNA and DNA.

In some embodiments of the method, the third polynucleotide comprises a splint or bridging polynucleotide, wherein the 5′-terminal sequence of the first polynucleotide strand and the 3′-terminal sequence of the second polynucleotide strand hybridize adjacent to one another on the splint or bridging polynucleotide to position the 5′-end of the first polynucleotide strand adjacent to the 3′-end of the second polynucleotide strand. In some embodiments, the splint or bridging polynucleotide is RNA, DNA, or a mixture of RNA and DNA.

In some embodiments, one or more additional polynucleotide strands can be hybridized to the third polynucleotide strand, where an additional polynucleotide strand hybridizes adjacent to the 3′-end of the first polynucleotide strand, or adjacent to the 5′-end of the second polynucleotide strand, or two additional polynucleotide strands hybridize adjacent to the 3′-end of the first polynucleotide strand and adjacent to the 5′-end of the second polynucleotide strand to form nick(s) that can be ligated with an engineered RNA ligase. Additional polynucleotide strands can be hybridized in like manner and ligated to prepare longer polynucleotide strands.

In some embodiments, the first polynucleotide strand and/or second polynucleotide strand can have a single strand region (e.g., overhang or unpaired regions) when hybridized to the third polynucleotide strand, and a fourth polynucleotide strand added that hybridizes to the single stranded region of the first polynucleotide strand or second polynucleotide stranded, where the fourth polynucleotide strand hybridizes adjacent to the 5′-end or 3′-end of the third polynucleotide strand to form a nick, which can be then ligated with an engineered RNA ligase. In some embodiments, the fourth polynucleotide strand can have a single stranded region (e.g., overhang or unpaired regions) when hybridized to the first polynucleotide strand or second polynucleotide strand on the third polynucleotide strand, and a fifth polynucleotide strand added that hybridizes to the single stranded region of the fourth polynucleotide strand, where the fifth polynucleotide strand hybridizes adjacent to the 3′-end of the first polynucleotide strand or the 5′-end of the second polynucleotide strand to form a nick, which can be ligated with an engineered RNA ligase. Such successive additions of polynucleotide strands can be used to generate longer polynucleotide products (see, e.g., Paul et al., ACS Chem. Biol. 2023, 18, 2183-2187, incorporated by reference herein).

In some embodiments, the first polynucleotide strand is hybridized to the third polynucleotide strand to form a first double stranded fragment, and the second polynucleotide strand is hybridized to a sixth polynucleotide strand or another polynucleotide strand to form a second double stranded fragment, where the first and second double stranded fragments can base pair to form a substrate for the engineered RNA ligase. In some embodiments, the first double stranded fragment and the second double stranded fragment have complementary overhangs or complementary single stranded ends, also referred to as cohesive or sticky ends, that can base pair and form double stranded nick(s) between the first and second double stranded fragments and serve as substrate(s) for the engineered RNA ligase.

In some embodiments, the complementary ends (e.g., sticky or cohesive ends) are of sufficient length for the double stranded fragments to base pair and form suitable substrates for the engineered RNA ligase. In some embodiments, the complementary single stranded end comprises at least 1-50, 2-40, 3-35, 4-30, 5-25, 6-20, or 8-15 or more nucleotides in length. In some embodiments, the complementary single stranded end is from 1-10, 2-8, or 4-6 nucleotides in length. In some embodiments, the complementary single stranded end is 1, 2, 3, 4, 5, 6, 8, 10, 12, 14, 16, 18, 20, or more nucleotides in length.

In some embodiments, the double stranded region or complementary portion of each of the double stranded fragment is from 4-50, 6-45, 8-40, 10-35, 12-30, or 14-25 nucleotides in length. In some embodiments, the double stranded region is at least 4, 5, 6, 7, 8, 9, 10, 12, 14, 16, 18, 20, 22, 24, 26, 28, 30, 35, 40, 45, or 50 nucleotides in length.

In some embodiments of the method, the ligase substrates comprise at least 2, 3, 4, 5, or 6 or more double stranded fragments, where each double stranded fragment has a complementary end that can base pair with at least one other double stranded fragment with a complementary end to form substrates for the RNA ligase. In some embodiments, the double stranded fragments have complementary or cohesive ends, e.g., at the 5′ and 3′ ends, such that the double stranded fragments can be ligated to form concatemers. In some embodiments, where the number of double stranded fragments is 3, at least one of the double stranded fragments has complementary ends at the 5′ and 3′ ends of the fragment, where double stranded fragment base pairs with two other different double stranded fragments to form a substrate that can be ligated to a product containing the three double stranded fragments. It is to be understood that where 3 or more double stranded fragments are ligated, the complementary ends of the double stranded fragments can be designed for ligation of all of the different double stranded fragments.

In some embodiments of the method, the first polynucleotide strand, second polynucleotide strand, third polynucleotide strand, fourth polynucleotide strand, fifth polynucleotide strand, sixth polynucleotide strand and/or another polynucleotide strand comprise one or more modified nucleotides or nucleotide analogs, wherein the modified nucleotide or nucleotide analog comprises a nucleobase analog or modified nucleobase, modified nucleoside sugar residue, modified internucleoside linkage, modified 5′-end phosphate group, and/or modified 3′-end hydroxyl group.

In some embodiments, the polynucleotide substrate comprises a modified 5′-end phosphate group, wherein the modified phosphate group is a phosphate analog. In some embodiments of the method, the first polynucleotide comprises a 5′-phosphate analog. In some embodiments, the 5′-phosphate analog is a phosphorothioate, phosphoramidate, monomethylphosphate, methylphosphonate, vinyl phosphonate, or phosphonocarboxylate.

In some embodiments of the method, the polynucleotide substrate comprises one or more modified nucleoside sugar residues. In some embodiments of the method, the first polynucleotide and/or second polynucleotide comprises one or more modified nucleoside sugar residues. In some embodiments, the modified nucleoside sugar residue is a 2′-O-alkyl, a 2′-halo, a B-D-ribo LNA, or a a-L-ribo-LNA (e.g., locked nucleic acids). In some embodiments, the modified nucleoside sugar residue is, among others, a 2′-O-methyl, 2′-O-ethyl, 2′-O-propyl group, or 2′-O-methoxyethyl. In some embodiments, the modified nucleoside sugar residue is 2′-fluoro, 2′-bromo, or 2′-chloro, preferably 2′-fluoro. In some embodiments, the sugar residue is modified with a conjugate, such as a targeting moiety, for example, GalNAc or lipid moieties.

In some embodiments of the method, the polynucleotide substrate comprises one or more modified nucleotide residues having a modified nucleobase or nucleobase analog. In some embodiments of the method, the first polynucleotide and/or second polynucleotide comprises one or more modified nucleotide residues having a modified nucleobase or nucleobase analog. In some embodiments, the nucleobase analog is, among others, xanthine, hypoxanthine, inosine, 6-methyladenine, 7-methylguanine, 2,6-diaminopurine, 5-methylcytosine, 5-hydroxycytosine, 5-bromocytosine, 5-iodocytosine, 2-thiothymine, 5-fluorouracil, 5-bromouracil, 8-bromoguanine, 8-aminoguanine, or 8-aza-7-deazaguanine. In some embodiments, the nucleobase is modified with a conjugate, such as a targeting moiety, for example, GalNAc and lipid moieties.

In some embodiments of the method, the polynucleotide substrate comprises one or more modified or non-standard internucleoside linkages, i.e., internucleoside linkages other than phosphate linkages. In some embodiments, the first polynucleotide and/or second polynucleotide comprises one or more modified or non-standard internucleoside linkages. In some embodiments, the internucleoside linkage is a phosphorothioate, phosphorodithioate, phosphoacetate, phosphoramidate, methylphosphonate, or phosphonocarboxylate. In some embodiments, at least 1%, 2%, 5%, 10%, or more of the internucleoside linkages are non-standard internucleoside linkages. In some embodiments, the non-standard internucleoside linkages are present 1, 2, 3, 4 or 5 nucleotides of the 5′ and/or 3′-terminus of the polynucleotide substrates.

In some embodiments of the method, the polynucleotide substrate comprises modified 3′-hydroxyl group. In some embodiments, 3′-end of a polynucleotide substrate is attached to a matrix or surface, such as a solid matrix. In such instances, the polynucleotide substrate attached to a matrix has a ligatable 5′-end. In some embodiments, the 3′-end of a polynucleotide substrate is modified with an amine, halo, phosphate, phosphate analog, —O-alkyl, lipid moiety, or a detectable label.

In some embodiments, the polynucleotide substrate comprises C-4′ modifications, including among others, 4′-thiol-C2′ modifications, 4′/5′ aminoalkyl/C2′ modifications, C4′-Guanidino-C2′-modifications, and C4′—O-Me/C2′-modifications (see, e.g., Gangopadhyay et al., RNA Biology, 2022, 19:1, 452-467.

In some embodiments, the polynucleotide substrate comprises a 2′-modified sugar moiety on the 3′-terminal nucleotide and/or 5′-terminal nucleotide, where the 3′-terminal nucleotide on the polynucleotide acceptor strand is being ligated to the 5′-terminal nucleotide of the polynucleotide donor strand substrates.

In some embodiments, the polynucleotide substrate comprises a modified nucleoside at the 3′-terminal nucleoside of the polynucleotide acceptor strand. In some embodiments, the polynucleotide acceptor strand comprises a modified 3′-terminal nucleoside of 2′-fluoro-adenosine, 2′-fluoro-guanosine, 2′-fluoro-cytidine, 2′-fluoro-uridine, 2′-fluoro-thymidine, 2′-O-methyl-adenosine, 2′-O-methyl-guanosine, 2′-O-methyl-cytidine, 2′-O-methyl-uridine, or 2′-O-methyl-thymidine.

In some embodiments, the polynucleotides substrate comprises a modified nucleoside at the 5′-terminal nucleoside of the polynucleotide donor strand. In some embodiments, a polynucleotide donor strand comprises a modified 5′-terminal nucleoside of 2′-fluoro-adenosine, 2′-fluoro-guanosine, 2′-fluoro-cytidine, 2′-fluoro-uridine, 2′-fluoro-thymidine, 2′-O-methyl-adenosine, 2′-O-methyl-guanosine, 2′-O-methyl-cytidine, 2′-O-methyl-uridine, or 2′-O-methyl-thymidine.

In some embodiments, the polynucleotide substrate comprises a polynucleotide acceptor strand having a modified 3′-terminal nucleoside of 2′-fluoro-adenosine, 2′-fluoro-guanosine, 2′-fluoro-cytidine, 2′-fluoro-uridine, 2′-fluoro-thymidine, 2′-O-methyl-adenosine, 2′-O-methyl-guanosine, 2′-O-methyl-cytidine, 2′-O-methyl-uridine, or 2′-O-methyl-thymidine, and a polynucleotide donor strand having a modified 5′-terminal nucleoside of 2′-fluoro-adenosine, 2′-fluoro-guanosine, 2′-fluoro-cytidine, 2′-fluoro-uridine, 2′-fluoro-thymidine, 2′-O-methyl-adenosine, 2′-O-methyl-guanosine, 2′-O-methyl-cytidine, 2′-O-methyl-uridine, or 2′-O-methyl-thymidine.

In some embodiments, the polynucleotide substrate comprises a polynucleotide acceptor strand having a modified 3′-terminal nucleoside of 2′-fluoro-adenosine, 2′-fluoro-guanosine, 2′-fluoro-cytidine, 2′-fluoro-uridine, 2′-fluoro-thymidine, 2′-O-methyl-adenosine, 2′-O-methyl-guanosine, 2′-O-methyl-cytidine, 2′-O-methyl-uridine, or 2′-O-methyl-thymidine; a polynucleotide donor strand having a modified 5′-terminal nucleoside of 2′-fluoro-adenosine, 2′-fluoro-guanosine, 2′-fluoro-cytidine, 2′-fluoro-uridine, 2′-fluoro-thymidine, 2′-O-methyl-adenosine, 2′-O-methyl-guanosine, 2′-O-methyl-cytidine, 2′-O-methyl-uridine, or 2′-O-methyl-thymidine; and a polynucleotide complementary to the polynucleotide acceptor strand and polynucleotide donor strand, wherein the polynucleotide complementary to the polynucleotide acceptor strand and polynucleotide donor strand comprises a complementary 2′-fluoro or 2′-O-methyl nucleoside at the position complementary to the 3′-terminal nucleoside of the polynucleotide acceptor strand, and/or a complementary 2-fluoro or 2-O-methyl nucleoside at the position complementary to the 5′-terminal nucleoside of the polynucleotide donor strand.

In some embodiments, the method includes a nucleotide cofactor or substrate used by the RNA ligase to catalyze the joining reaction. In some embodiments, the nucleotide cofactor or substrate is ATP and/or dATP. Preferably, the nucleotide cofactor or substrate is ATP. In some embodiments, the reaction conditions for the ligation include additional components, such as Mg+2, buffer, and/or salts.

In some embodiments, the nucleotide cofactor or substrate (e.g., ATP) is present at a concentration of about 0.05-25 mM, 1-20 mM, 2-18 mM, or 5-15 mM. In some embodiments, the nucleotide cofactor or substrate is present at a concentration of about 0.5 mM, 1 mM, 2 mM, 3 mM 4 mM, 5 mM, 6 mM, 7 mM, 8 mM, 9 mM, 10 mM, 12 mM, 15 mM, 20 mM, or 25 mM.

In some embodiments, the reaction conditions also include a ligation enhancing reagent, including, among others, DMSO, betaine, polyethylene glycol (e.g., PEG 6000, PEG 8000, etc.), bovine serum albumin, Ficoll, and dextran (e.g., Dextran 6000).

In some embodiments, ligation reaction is carried out at a suitable temperature and reaction time period. In some embodiments, the ligation reaction temperature is from about 2° C. to about 60° C. In some embodiments, the ligation reaction temperature is from 4° C. to 55° C., 4° C. to 50° C., 4° C. to 45° C., or 10° C. to 40° C. In some embodiments, the ligation reaction temperature is 2° C., 5° C., 10° C., 15° C., 20° C., 25° C., 30° C., 37° C., 40° C., 45° C., 50° C., 55° C., or 60° C. In some embodiments, the ligation reaction temperature can use different temperatures, for example a temperature at which stable hybrids are formed between polynucleotide substrate(s) followed by a higher temperature to promote completion of ligation reaction.

In some embodiments, the ligation reaction time can be a sufficient time for ligation of polynucleotide substrate(s). In some embodiments, the ligation reaction time is from 0.5-72 hr or longer. In some embodiments, the ligation reaction time is 1-72 hr, 2-48 hr, or 2-24 hr. In some embodiments, the ligation reaction time is 0.5, 1, 2, 4, 5, 12, 24, 48, or 72 hr or longer.

In some embodiments, the ligase reaction is carried out at a suitable pH. In some embodiments, the pH of the ligase reaction is about pH 5-9, about pH 5.5-8.5, about pH 6-8, or about pH 6.5-7.5. In some embodiments, the pH of the ligase reaction is about pH 5, about pH 5.5, about pH 6, about pH 6.5, about pH 7, about pH 7.5, about pH 8, about pH 8.5 or about pH 9.

In some embodiments, the RNA ligase substrate concentration is at about 0.01-1 mM, 0.05-0.9 mM, 0.1-0.8 mM, 0.2-0.7 mM, or 0.3 mM-0.6 mM. In some embodiments, the RNA ligase substrate is at about 0.01 mM, 0.05 mM, 0.1 mM, 0.2 mM, 0.3 mM, 0.4 mM, 0.5 mM, 0.6 mM, 0.7 mM, 0.8 mM, 0.9 mM or 1 mM. In some embodiments, the RNA ligase substrate concentration is at about 1-10 mM, 2-8 mM, or 4-6 mM. In some embodiments, the engineered RNA ligase exhibits increased product yield at about 1 mM, 2 mM, 3 mM, 4 mM, 5 mM, 6 mM, 7 mM, 8 mM, 9 mM, or 10 mM.

In some embodiments, the polynucleotide substrate is bound to a support, and the ligation reaction carried out with the engineered RNA ligase to ligate a polynucleotide substrate to the support bound polynucleotide, or ligate one or more polynucleotide substrates hybridized to the support-bound polynucleotide substrate.

In some embodiments, the ligation reaction is carried out with an engineered RNA ligase bound to a support. In some embodiments, the polynucleotide substrates in solution can be contacted with the support-bound RNA ligase to ligate the polynucleotide substrates. In some embodiments, the polynucleotide substrates in solution are passed through a bed of support-bound engineered RNA ligase until sufficient ligated product has formed.

In some embodiments, the ligase reaction further includes a nucleotide substrate regeneration system to regenerate co-factor substrate ATP (or dATP). In some embodiments, the ATP recycling or regeneration system includes a nucleoside monophosphate kinase for converting AMP to ADP in presence of a phosphate donor. Various nucleoside monophosphate kinase can be used for the conversion of AMP to ADP, including homologs of nucleoside monophosphate kinases. In some embodiments, more than one nucleoside monophosphate kinase can be used in the regeneration system. In some embodiments, the nucleoside monophosphate kinase is an adenosine monophosphate kinase (e.g., adenylate kinase), cytidine monophosphate (CMP) kinase, uridine monophosphate (UMP) kinase, or guanylate-monophosphate (GMP) kinase.

In some embodiments, a nucleoside monophosphate kinase useful in the enzymatic reactions is a cytidine monophosphate kinases. Various suitable cytidine monophosphate kinases are known in the art. These include homologs of cytidine monophosphate kinases. In some embodiments, a cytidine monophosphate kinase useful in the enzymatic reactions includes, among others, the cytidine monophosphate kinase of Thermus thermophilus (Q5SL35), Pyrococcus furiosus (Q8U2L4), Pseudomonas putida (AFO48857.1), Escherichia coli K-12 MG1655 (POA610), Clostridium acetobutylicum (Q97108), Halobacterium salinarum (Q9HPA5) Bacillus acidicola (WP_066270173), Acetobacter aceti (WP_010667744), Acidithiobacillus thiooxidans (WP_024892761.1), Acidithiobacillus ferrooxidans (WP_064220349.1), Metallosphaera sedula (WP_011921264.1), Amphibacillus xylanus (WP_015009966.1) Thioalkalivibrio denitrificans (WP_077278466.1), Vibrio psychroerythus (Q482G4), Pseudoalteromonas haloplanktis (Q3ILA1), Psychrobacter arcticus (Q4FRL5), Psychromonas ingrahamii (A1SZ01), Pseudomonas syringae (xQ4ZQ97) and Halobacterium salinarum (Q9HPA5).

In some embodiments, a nucleoside monophosphate kinase useful in the enzymatic reactions is a uridine monophosphate kinase. Various suitable uridine monophosphate kinases are known in the art. These include homologs of uridine monophosphate kinases. In some embodiments, a uridine monophosphate kinase useful in the enzymatic reactions includes, among others, the uridine monophosphate kinase of Pyrococcus furiosus (Q8U122), Thermus thermophilus (P43891), Pseudomonas putida (I7BW46), Escherichia coli K-12 MG1655 (POATE9), Aspergillus niger (A2R195), Saccharomyces cerevisiae (P15700), Clostridium acetobutylicum (Q97164) ATCC 824 PyrH Halobacterium salinarum (Q9HNN8), Picrophilus torridus (WP_048059653), Metallosphaera sedula (WP_012021705), Thermoplasma acidophilum (WP_010900913), Sulfolobus solfataricus (WP_009992427), Acetobacter aceti (WP_042788648), Thioalkalivibrio sp. HK1 (WP_081759172.1), Amphibacillus xylanus (WP_015010200.1), Vibrio psychroerythus (Q485G8), Pseudoalteromonas haloplanktis (Q3IIX6), Psychrobacter arcticus (Q4FRH5), Psychromonas ingrahamii (ABM04676.1), Pseudomonas syringae (Q4ZWS6), and Halobacterium salinarum (Q9HNN8).

In some embodiments, the nucleoside monophosphate kinase useful in the enzymatic reactions is a guanosine monophosphate kinase (guanylate kinase). Various suitable guanylate kinases are known in the art. These include homologs of guanylate kinases. In some embodiments, a guanylate kinase useful in the enzymatic reactions includes, among others, the guanylate kinase of Thermotoga maritima (Q9X215), Thermus thermophilus (Q5SI18), Pseudomonas putida (I7C087), Escherichia coli K-12 (P60546), Aspergillus niger (A2QPV2), Saccharomyces cerevisiae (P15454), Clostridium acetobutylicum (Q97IDO), Acidithiobacillus ferrooxidans (WP_064219869.1), Acidithiobacillus thiooxidans (WP_010637919.1), Bacillus acidicola (WP_066264774.1), Acetobacter aceti (WP_018308252.1), Amphibacillus xylanus (WP_015010280.1), Thioalkalivibrio sulfidiphilus (WP_018953989.1), Vibrio psychroerythus (Q47UB3), Pseudoalteromonas haloplanktis (Q3IJH8), Psychrobacter arcticus (Q4FQY7), Psychromonas ingrahamii (A1TOP1), and Pseudomonas syringae (Q4ZZY8).

In some embodiments, the nucleoside monophosphate kinase useful in the enzymatic reactions is an adenosine monophosphate kinase (adenylate kinase). Various suitable adenylate kinases are known in the art. These include homologs of adenylate kinases. In some embodiments, the adenylate kinase is a bacterial, fungal, plant, or animal adenylate kinase. In some embodiments, an adenylate kinase useful in the enzymatic reactions includes, among others, adenylate kinases of Thermus thermophilus (Q72125), Pyrococcus furiosus (Q8U207), Pseudomonas putida (17CAA9), Escherichia coli K-12 W3110 (P69441), Aspergillus niger CBS 513.88 (A2QPN9), Saccharomyces cerevisiae (P07170), Clostridium acetobutylicum (Q97E39), Halobacterium salinarum (Q9HPAT), Acidithiobacillus thiooxidans (WP_024894015.1), Acidithiobacillus ferrooxidans (WP_064218420.1), Bacillus acidicola (WP_066267988.1), Sulfolobus solfataricus (WP_009991241.1), Saccharomyces cerevisiae (P07170), Thermotoga neapolitana (Q8GGL2), Escherichia coli (P69441) and Geobacillus stearothermophilus (WP_049624206.1). In some embodiments, the adenylate kinase is an engineered adenylate kinase described in International patent application No. PCT/US2024/051084, filed Oct. 11, 2024, incorporated herein by reference.

In some embodiments, the ATP regeneration system includes at least an enzyme for the conversion of ADP to ATP in presence of a phosphate donor. In some embodiments, the ATP regeneration system includes, among others, an acetate kinase, pyruvate kinase, creatine kinase, or polyphosphate kinase (see, e.g., Endo et al., Adv. Synth. Catal., 2002, 343:521-526; Andexer et al., Chem Bio Chem., 2015, 16:380-386). In the ATP regeneration system, the phosphate donor for the conversion of ADP to ATP is selected based on the ATP regenerating enzyme employed. By way of example and not limitation, if acetate kinase enzyme is used for conversion of ADP to ATP, the phosphate donor is acetyl-phosphate. If pyruvate kinase is used for the conversion of ADP to ATP, the phosphate donor is phosphoenolpyruvate. If creatine kinase is used for the conversion of ADP to ATP, the phosphate donor is creatine phosphate. If polyphosphate kinase is used for the conversion of ADP to ATP, the phosphate donor is inorganic polyphosphate.

Accordingly, in some embodiments, the ATP regenerating system includes pyruvate kinase and phosphoenolpyruvate. In some embodiments, the ATP regenerating system includes creatine kinase and creatine phosphate. In some embodiments, the ATP regenerating system includes polyphosphate kinase and inorganic polyphosphate. In some embodiments, the ATP regenerating system includes acetate kinase and acetyl phosphate. In some embodiments, the acetate kinase is an acetate kinase of Escherichia coli str. K-12 substr. MG1655 (NP_416799.1), Corynebacterium jeikeium K411 (WP_011272972.1), Lactococcus cremoris subsp. cremoris KW2 (WP_011835968.1), Lactococcus lactis (WP_004254593.1), Marinitoga sp. 38H-ov (WP_165147355.1), Thermotoga sp. KOL6 (WP_101510533.1), Thermosipho melaniensis (WP_012057479.1), Thermotoga sp. RQ7 (WP_041844042.1), and Thermosipho africanus (WP_004102380.1). In some embodiments, the acetate kinase is an engineered acetate kinase described in International patent application No. PCT/US2024/051118, filed Oct. 11, 2024, incorporated by reference herein.

In some embodiments, the engineered RNA ligase is used to ligate nicks or related nick structures (see, e.g., Cheng et al., Royal Soc Chem Adv., 2019, 9:8620-8627). In some embodiments, the engineered RNA ligase is used to synthesize RNAs from shorter oligonucleotides, for example by use of splint nucleic acids (see, e.g., Stark et al., RNA, 2006, 12:2014-2019). In some embodiments, the engineered RNA ligase is used to ligate modified oligonucleotides, such as provided in the Examples. Other examples of modified oligonucleotide products that can be synthesized using short oligonucleotide substrates, include, among others, shRNAs or siRNAs described in patent publications WO22104366, WO22029209, WO22031847, WO20226960, US2022072024, US2021238606, US11286488, US2017305956, WO22212153, WO22192519, WO22125490, WO22072447, WO21257568, WO21102373, WO21072395, WO21022108, US2022079971, US11034957, US2021332365, US11015201, US10995336, US11091759, US10889813, US10130651, US10513703, WO19232255, WO21108640, WO22147304, and WO21138537.

### Method for Predicting Activity Profile for an RNA Ligase and Ligase Substrate

In another aspect, the present disclosure provides a method of assessing a ligation activity profile for a RNA ligase, comprising obtaining an RNA ligase enzyme and a RNA ligase substrate ligation activity data for multiple reaction variables, performing or applying Gaussian Process Regression (GPR) on the ligation activity data for the multiple reaction variables, and out putting the predicted activity distribution for the multiple reaction variables. The method further comprises predicting the optimal ligation condition for the RNA ligase and RNA ligase substrate. In some embodiments, the output comprises a three dimensional ligase activity map.

In some embodiments, the multiple reaction variables include, among others, cofactor nucleotide (e.g., ATP) concentration, divalent metal concentration (e.g., MgCl₂), ligase substrate concentration, 5-terminal nucleotide at the ligation junction (i.e., 5′-nucleotide of the nick), 3′-terminal nucleotide at the ligation junction (3′-nucleotide of the nick), 5′-terminal region sequence of the ligation junction, 3′-terminal sequence of the ligation junction, and ligation temperature. In some embodiments, the multiple reaction variable further include type of modification of the 3′-terminal nucleotide of the ligation junction and/or modification of the 5′-terminal nucleotide of the ligation junction.

In some embodiments, the Gaussian Process Regression analysis is carried out for each different RNA ligase being examined for a defined ligase substrate. In some embodiments, a Gaussian Process Regression analysis is developed for each RNA ligase for a set of different ligase substrates.

### Kits

In a further aspect, the present disclosure provides a kit comprising an engineered RNA ligase described herein. In some embodiments, the kit further comprises one or more of a buffer, a nucleotide substrate (e.g., ATP or dATP), and/or one or more polynucleotide substrates. In some embodiments, the kit further comprises an additive or ligation enhancing agent, including but not limited to, one or more of DMSO, betaine, polyethylene glycol (e.g., PEG 6000, PEG 8000, etc.), bovine serum albumin, Ficoll, and dextran (e.g., Dextran 6000).

### EXAMPLES

The following Examples, including experiments and results achieved, are provided for illustrative purposes only and are not to be construed as limiting the present invention.

In the experimental disclosure below, the following abbreviations where relevant apply: ppm (parts per million); M (molar); mM (millimolar), uM and μM (micromolar); nM (nanomolar); mol (moles); gm and g (gram); mg (milligrams); ug and μg (micrograms); L and 1 (liter); ml and mL (milliliter); ul, uL, μl, and μL (microliter); cm (centimeters); mm (millimeters); um and μm (micrometers); sec. (seconds); min(s) (minute(s)); h(s) and hr(s) (hour(s)); U (units); OD (optical density); MW (molecular weight); rpm (rotations per minute); rcf (relative centrifugal force); psi and PSI (pounds per square inch); ° C. (degrees Celsius); RT and rt (room temperature); NGS (next-generation sequencing); ds (double stranded); ss (single stranded); CDS (coding sequence); DNA (deoxyribonucleic acid); RNA (ribonucleic acid); E. coli W3110 (commonly used laboratory E. coli strain, available from the Coli Genetic Stock Center [CGSC], New Haven, CT); HTP (high throughput); HPLC (high pressure liquid chromatography); FPLC (fast protein liquid chromatography); ddH₂O (double distilled water); PBS (phosphate buffered saline); BSA (bovine serum albumin); DTT (dithiothreitol); CAM (chloramphenicol); CAT (chloramphenicol acetyltransferase); IPTG (isopropyl β-D-1-thiogalactopyranoside); FIOPC or FIOP (fold improvements over positive control or parent); LB (Luria-Bertani); TB (Terrific-Broth).

### Example 1

### E. coli Expression Hosts Containing Recombinant RNA Ligase 2 (Rnl2) Genes

The initial RNA ligase 2 enzyme used to produce the variants of the present invention was SEQ ID NO: 2 cloned into a pJV110900 expression vector (see, e.g., U.S. Pat. No. 10,184,117) operatively linked to the lac promoter under control of the lacl repressor. The expression vector also contains the P15a origin of replication and the chloramphenicol resistance gene. The resulting plasmids were transformed into E. coli W3110, using standard methods known in the art. The transformants were isolated by subjecting the cells to chloramphenicol selection, as known in the art (see e.g., U.S. Pat. No. 8,383,346 and International patent publication WO2010/144103).

### Example 2

### Preparation of RNA ligase (Rnl2)-Containing Wet Cell Pellets

E. coli cells containing recombinant RNA ligase 2-encoding genes from monoclonal colonies were inoculated into 180 uL LB containing 1% glucose and 30 μg/mL chloramphenicol (CAM) in the wells of 96-well, shallow-well microtiter plates. The plates were sealed with O₂-permeable seals, and cultures were grown overnight at 30° C., 200 rpm, and 85% humidity. Then 10 μL of each of the cell cultures were transferred into the wells of 96-well, deep-well plates containing 390 mL TB and 30 μg/mL CAM. The deep-well plates were sealed with O₂-permeable seals and incubated at 30° C., 250 rpm, and 85% humidity until OD₆₀₀ 0.6-0.8 was reached. The cell cultures were then induced by IPTG to a final concentration of 1 mM and incubated overnight under the same conditions as originally used. The cells were then pelleted using centrifugation at 4,000 rpm for 10 min. The supernatants were discarded, and the pellets were frozen at −80° C. prior to lysis.

### Example 3

### Shake Flask Expression and Purification of RNA Ligase 2 (Rnl2)

### Shake Flask Expression

Selected variant cultures grown as described above were plated onto LB agar plates with 1% glucose and 30 μg/mL chloramphenicol and grown overnight at 37° C. A single colony from each culture was transferred to 5 mL LB broth with 1% glucose and 30 μg/ml chloramphenicol. The cultures were grown for 20 h at 30° C. and 250 rpm, and then 30 μL of each culture was used to inoculate 160 mL of Terrific Broth containing 0.075% (w/v) glucose, 0.03% (w/v) lactose, and 30 μg/ml of chloramphenicol in a 1 L baffled shake flask. The cultures were incubated for 20 h at 30° C. and 250 rpm. Then the cultures were centrifuged at 4000 rpm for 10 min. The culture supernatant was discarded and the pellets were resuspended in 30 mL lysis buffer comprised of 50 mM Tris-HCl at pH 7.5. This cell suspension was chilled in an ice bath and lysed using a Microfluidizer cell disruptor (Microfluidics M-110L). The crude lysate was pelleted by centrifugation (11,000 rpm for 60 min at 4° C.), and the supernatant was then filtered through a 0.2 μm PES membrane to further clarify the lysate.

Purification of RNA Ligase from Shake Flask Lysates

Additional NaCl and imidazole were added to the clarified RNA ligase lysates to adjust the concentrations to 500 mM NaCl and 20 mM imidazole. Lysates were then purified using an AKTA Pure purification system and a 5 ml HisTrap FF column (GE Healthcare) using the AC Step HiF setting (the run parameters are provided below in Table 3.1). The wash buffer comprised 50 mM Tris-HCl pH 8, 500 mM NaCl, 20 mM imidazole, and the elution buffer comprised 50 mM Tris-HCl, pH 8, 500 mM NaCl, 250 mM imidazole.

TABLE 3.1
Purification Parameters

| Parameter | Volume | |
|---|---|---|
| Column volume | 5 | mL |
| Flow rate | 5 | mL/min |
| Pressure limit | 0.3 | MPa |
| Sample volume | 45 | ml |
| Equilibration volume | 5 column volumes (CV) = 25 mL | |
| Wash Unbound volume | 15 CV = 75 mL | |
| Elution | Isocratic (step) | |
| Elution volume | 5 CV = 25 mL | |
| Fraction volume | 1.5 | mL |

| | |
|---|---|
| RE-equilibration volume | 5 CV = 25 mL |

The first 2 elution fractions with over 1 mg/mL protein were identified by absorption at 280 nm on a NanoDrop Microvolume Spectrophotometer and dialyzed overnight in dialysis buffer comprised of 10 mM Tris-HCl pH 7.5, 50 mM KCl, 50% glycerol in a 3.5 K Slide-A-Lyzer™ dialysis cassette (Thermo Fisher) for buffer exchange. Concentrations of purified and dialyzed RNA polymerase samples were measured by absorption at 280 nm.

### Example 4

### Capillary Electrophoresis (CE) Analysis of Oligonucleotides

### Sample Preparation for Reaction Analysis Using CE

For analysis of the reaction samples, capillary electrophoresis was performed using an ABI 3500xl Genetic Analyzer (ThermoFisher). Reactions were quenched by the addition of water and EDTA such that the final concentration of EDTA in the quenched reaction was 20 mM. Quenched reactions were diluted in water to approximately 1.25 nM oligonucleotide, and a 2-uL aliquot of this solution was transferred to a new 96-well MicroAmp Optical PCR plate or 384-well MicroAmp Optical PCR plate containing 18 uL Hi-Di™ Formamide (ThermoFisher) containing an appropriate size standard (LIZ or Alexa633). The ABI3500xl was configured with POP6 polymer, 50 cm capillaries, and a 55° C. oven temperature. Pre-run settings were 18 KV for 50 sec. Injection was 10 KV for 2 sec, and the run settings were 19 KV for 620 sec. FAM-labeled oligo substrates and products were identified by their sizes relative to the sizing ladder.

### List of Oligonucleotide Substrates and Products

The oligonucleotides in Table 4.1 were used to assay RNA ligase activity. Each of the oligonucleotides are in the 5′-3′ direction, and the oligonucleotide residues are either deoxyribonucleic acid residues or their derivatives. FAM is fluorescein amidite. The oligonucleotides in Table 4.1 have the following features. Sense strand fragment 1 (AS017) has a 2′-methoxy group at residue 5 and 6, a 5′-OH terminus, and a 3′-OH terminus; sense strand fragment 2 (AS023) has a 5′-phosphate terminus and a 3′-FAM terminus; sense strand fragment 3 (AS032) has a 5′-phosphate terminus and a 3′-OH terminus; sense strand fragment 4 has a 5′-phosphate terminus, a 3′-FAM terminus, and a 2′-methoxy group at residue 5 and 6. Sense strand fragment 5 (AS047) has a 2′-methoxy group at residue 5 and 6, a 5′-OH terminus, and a 3′-OH terminus; antisense strand fragment 2 (AS090) has a 5′-phosphate terminus and a 3′-FAM terminus; antisense strand fragment 2 (AS092) has a 5′-phosphate terminus; antisense strand fragment 4 (AS040) has a 2′-methoxy group at residue 8 and a 2′-fluoro group at residue 9. For Reaction 1, sense strand fragment 2 (AS023) and 3 (AS032) were first mixed at a ratio of 1:100, and then mixed with the sense strand fragment 1 (AS017) and the antisense strand fragment 1 (AS022) at equal molar ratio at 3.2 mM stock concentration. For Reaction 2, antisense strand fragment 2 (AS090) and antisense strand fragment 3 (AS092) were mixed at a ratio of 1:100, and the sense strand fragment 2 (AS023) and 3 (AS032) were mixed at a ratio of 1:100, and then these mixtures were further mixed with the sense strand fragment 5 (AS047) and antisense strand fragment 4 (AS040) at equal molar ratio at 2.5 mM stock concentration. For Reaction 3, antisense strand fragment 6 (AS103) and antisense strand fragment 7 (AS104) were mixed at a ratio of 1:100, and the sense strand fragment 7 (AS100) and 8 (AS101) were mixed at a ratio of 1:100, and then these mixtures were further mixed with the sense strand fragment 6 (AS099) and antisense strand fragment 5 (AS102) at equal molar ratio at 2.5 mM stock concentration. For Reaction 4, antisense strand fragment 9 (AS109) and antisense strand fragment 10 (AS118) were mixed at a ratio of 1:100, and the sense strand fragment 9 (AS108) and 10 (AS117) were mixed at a ratio of 1:100, and then these mixtures were further mixed with the sense strand fragment 6 (AS099) and antisense strand fragment 8 (AS110) at equal molar ratio at 2.5 mM stock concentration. For Reaction 3, antisense strand fragment 6 (AS103) and antisense strand fragment 7 (AS104) were mixed at a ratio of 1:100, and the sense strand fragment 7 (AS100) and 8 (AS101) were mixed at a ratio of 1:100, and then these mixtures were further mixed with the sense strand fragment 6 (AS099) and antisense strand fragment 5 (AS102) at equal molar ratio at 2.5 mM stock concentration. For Reaction 4, antisense strand fragment 9 (AS109) and antisense strand fragment 10 (AS118) were mixed at a ratio of 1:100, and the sense strand fragment 9 (AS108) and 10 (AS117) were mixed at a ratio of 1:100, and then these mixtures were further mixed with the sense strand fragment 6 (AS099) and antisense strand fragment 8 (AS110) at equal molar ratio at 2.5 mM stock concentration. For Reaction 5, antisense strand fragment 12 (AS116) and antisense strand fragment 13 (AS114) were mixed at a ratio of 1:100, and the sense strand fragment 12 (AS115) and 13 (AS112) were mixed at a ratio of 1:100, and then these mixtures were further mixed with the sense strand fragment 11 (AS111) and antisense strand fragment 11 (AS113) at equal molar ratio at 2.5 mM stock concentration. For Reaction 6, antisense strand fragment 15 (AS124) and antisense strand fragment 16 (AS121) were mixed at a ratio of 1:100, and the sense strand fragment 15 (AS123) and 16 (AS120) were mixed at a ratio of 1:100, and then these mixtures were further mixed with the sense strand fragment 14 (AS119) and antisense strand fragment 14 (AS122) at equal molar ratio at 2.5 mM stock concentration. For Reaction 7, antisense strand fragment 18 (AS137) and antisense strand fragment 19 (AS135) were mixed at a ratio of 1:100, and the sense strand fragment 18 (AS136) and 19 (AS131) were mixed at a ratio of 1:100, and then these mixtures were further mixed with the sense strand fragment 17 (AS132) and antisense strand fragment 17 (AS133) and antisense strand fragment 20 (AS134) at equal molar ratio at 2.5 mM stock concentration.

TABLE 4.1
RNA ligase Oligonucleotide Substrates

| Ligase Substrate Name | Sequence |
|---|---|
| Sense strand fragment 1 (AS017) | 5′-CTAGmAmC |
| Sense strand fragment 2 (AS023) | 5′-p-CTGTTTTGCTTTTGT-3′FAM (SEQ ID NO: 1471) |
| Sense strand fragment 3 (AS032) | 5′-p-CTGTTTTGCTTTTGT (SEQ ID NO: 1472) |
| Antisense strand fragment 1 (AS022) | ACAAAAGCAAAACAGGTCTAGAA (SEQ ID NO: 1473) |
| Sense strand fragment 4 (AS046) | 5′-CTAGmAmCCTGTTTTGCTTTTGT-3′FAM (SEQ ID NO: |
| | 1474) |
| Sense strand fragment 5 (AS047) | 5′-CTAGmAmA |
| Antisense strand fragment 2 (AS090) | 5′-p-AAACAGTTCTAG-3′FAM (SEQ ID NO: 1475) |
| Antisense strand fragment 3 (AS092) | 5′-p-AAACAGTTCTAG (SEQ ID NO: 1476) |
| Antisense strand fragment 4 (AS040) | 5′-ACAAAAGfCmA |
| Sense strand fragment 6 (AS099) | 5′-mCmUmAmGmAmA |
| Sense strand fragment 7 (AS100) | 5′-p-mCmUmGmUmUmGmUmAmAmCmCmAmAmGmA-3′- |
| | FAM |
| Sense strand fragment 8 (AS101) | 5′-p-mCmUmGmUmUmGmUmAmAmCmCmAmAmGmA |
| Antisense strand fragment 5 (AS102) | 5′-mUmCmUmUmGmG |
| Antisense strand fragment 6 (AS103) | 5′-p- |
| | mUmUmAmCmAmAmCmAmGmUmUmCmUmAmGmAmA- |
| | 3′-FAM |
| Antisense strand fragment 7 (AS104) | 5′-p- |
| | mUmUmAmCmAmAmCmAmGmUmUmCmUmAmGmAmA |
| Sense strand fragment 9 (AS108) | 5′-p-fCmUfGmUfUmGmUmAmAmCmCmAmAmGmA-3′- |
| | FAM |
| Sense strand fragment 10 (AS117) | 5′-p-fCmUfGmUfUmGmUmAmAmCmCmAmAmGmA |
| Antisense strand fragment 8 (AS110) | 5′-mUfCmUmUmGfG |
| Antisense strand fragment 9 (AS109) | 5′-p-mUmUfAmCmAfAmCfAmGfUmUfCmUmAmGmAmA- |
| | 3′-FAM |
| Antisense strand fragment 10 (AS118) | 5′-p-mUmUfAmCmAfAmCfAmGfUmUfCmUmAmGmAmA |
| Sense strand fragment 11 (AS111) | 5′-mUmGmGmGmAmU |
| Sense strand fragment 12 (AS115) | 5′-p-fUmUfCfAfGmUfCmUfCmAmUmCmUmUmA-3′-FAM |
| Sense strand fragment 13 (AS112) | 5′-p-fUmUfCfAfGmUfCmUfCmAmUmCmUmUmA |
| Antisense strand fragment 11 (AS113) | 5′-mUfAfAfGmAfUmG |
| Antisense strand fragment 12 (AS116) | 5′-p-fAmGfAmCmUmGfAmAfAmUmCmCmCmAmUmC-3′- |
| | FAM |
| Antisense strand fragment 13 (AS114) | 5′-p-fAmGfAmCmUmGfAmAfAmUmCmCmCmAmUmC |
| Sense strand fragment 14 (AS119) | 5′-mCmAmGmAmAmA |
| Sense strand fragment 15 (AS123) | 5′-p-fGmAfGmUfCmUmGmGmAmAmAmUmAmUmA-3′- |
| | FAM |
| Sense strand fragment 16 (AS120) | 5′-p-fGmAfGmUfCmUmGmGmAmAmAmUmAmUmA |
| Antisense strand fragment 14 (AS122) | 5′-mUfAmUmAmUfU |
| Antisense strand fragment 15 (AS124) | 5′-p-mUfCfCmAmGfAmCfUmCfUmUfUmCfUmGmGmU-3′- |
| | FAM |
| Antisense strand fragment 16 (AS121) | 5′-p-mUfCfCmAmGfAmCfUmCfUmUfUmCfUmGmGmU |
| Sense strand fragment 17 (AS132) | 5′-p-fUmGmUmAmAmCmCmAmAmGmA |
| Sense strand fragment 18 (AS136) | 5′-FAM-mCmUmAmGmAmAfCmUfGmU |
| Sense strand fragment 19 (AS131) | 5′-mCmUmAmGmAmAfCmUfGmU |
| Antisense strand fragment 17 (AS133) | 5′-p-fUmUfCmUmAmGmAmA |
| Antisense strand fragment 18 (AS137) | 5′-FAM-mUfCmUmUmGfGmU |
| Antisense strand fragment 19 (AS135) | 5′-mUfCmUmUmGfGmU |
| Antisense strand fragment 20 (AS134) | 5′-p-mUfAmCmAfAmCfAmG |

### Example 5

### Improvements Over SEQ ID NO: 2 in RNA Ligase Activity

The RNA ligase of SEQ ID NO: 2 was selected as the parent enzyme in an RNA ligation assay. Libraries of engineered genes were produced using known techniques (e.g., saturation mutagenesis, recombination of previously identified beneficial mutations). The polypeptides encoded by each gene were produced in high throughput (HTP) as described in Example 1, and the cell paste was generated as described in Example 2. To prepare lysate, 300 ul of 0.1 mg/mL lysozyme in 50 mM Tris-HCl buffer at pH 7.5 was added to the cell paste of each sample. After thorough resuspension at room temperature on a plate mixer for 30 min, the cells were incubated at 40° C. for 1 hour in a PCR thermocycler. The samples were then centrifuged for 10 min at 4000 rpm and 4° C., and the clear supernatants were collected for ligation reactions.

The lysate was screened in a 100 nanoliter reaction that comprised 50 mM Tris-HCl buffer at pH 7.5, 2 mM MgCl₂, 1 mM dithiothreitol (DTT), 0.4 mM adenosine triphosphate (ATP), and 1.6 mM of an equimolar mixture of the oligonucleotide fragments compromising Reaction 1. Nanoliter scale liquid transfers were carried out by Echo Acoustic Liquid Handler. The reactions were mixed by vortex, incubated for 2 hr at 37° C. in a PCR thermocycler, and quenched and diluted for analytical analysis by CE as described in Example 4. Using the peak area from CE analytical method, the % conversion for each sample was calculated as shown below:

$$\% conversion = \frac{area under the curve for the product peak}{\begin{matrix}Total area under the curve for \\ all products and remaining fragments\end{matrix}} * 100$$

Activity relative to SEQ ID NO: 2 (Activity FIOP) was calculated as the % conversion by the variant over the % conversion by SEQ ID NO: 2. The result is shown in Table 5.1.

TABLE 5.1
Activities of Variants Relative to SEQ ID NO: 2

| | | Activity |
|---|---|---|
| | Amino Acid | FIOP |
| SEQ ID | Differences | Relative to |
| NO: | (Relative to | SEQ ID |
| (nt/aa) | SEQ ID NO: 2) | NO: 2 |
| 3/4 | K256A | ++++ |
| 5/6 | E258P | ++++ |
| 7/8 | K254L | ++++ |
| 9/10 | K251S | ++++ |
| 11/12 | K248T | ++++ |
| 13/14 | D37M | ++++ |
| 15/16 | F54L | ++++ |
| 17/18 | E258M | ++++ |
| 19/20 | T232S | ++++ |
| 21/22 | Y15M | +++ |
| 23/24 | P71T | +++ |
| 25/26 | K26L | +++ |
| 27/28 | T232M | +++ |
| 29/30 | G116L | +++ |
| 31/32 | M38S | +++ |
| 33/34 | K251L | +++ |
| 35/36 | N25S | +++ |
| 37/38 | S106G/K251F | +++ |
| 39/40 | T52C | +++ |
| 41/42 | G33L | +++ |
| 43/44 | K251G | +++ |
| 45/46 | K256S | +++ |
| 47/48 | A247L | ++ |
| 49/50 | D37L/S249R/V266L/M316L/T330R/Q334R/N335H | ++ |
| 51/52 | N21S | ++ |
| 53/54 | G11N/D37L/S249L/V266L/M316L | ++ |
| 55/56 | E261P | ++ |
| 57/58 | G11N/V266L/M316L | ++ |
| 59/60 | G11N/K248T/S249R/V266L | ++ |
| 61/62 | I331D | ++ |
| 63/64 | G11N/K248T/S249L | ++ |
| 65/66 | D37F | ++ |
| 67/68 | A255V | ++ |
| 69/70 | D37L/S249R/V266L/M316L/Q334R | ++ |
| 71/72 | M38L | ++ |
| 73/74 | V266L | ++ |
| 75/76 | T232R | ++ |
| 77/78 | G11N/D37L/T156Q/S249R/N335H | ++ |
| 79/80 | D37L/T156Q/K248T/S249R/V266L/Q334R | ++ |
| 81/82 | Q334E | ++ |
| 83/84 | T156Q | ++ |
| 85/86 | G11N/V266L/M316L/Q334R | ++ |
| 87/88 | G11N/H39P/T156Q/K248T/M316L | ++ |
| 89/90 | G11N/S249R | ++ |
| 91/92 | D37L/T156Q | ++ |
| 93/94 | V107M/K251R | ++ |
| 95/96 | T156Q/S249R/M316L | ++ |
| 97/98 | D37L/T156Q/M316L | ++ |
| 99/100 | G11N/D37L/T156Q/S249L | ++ |
| 101/102 | G11N/D37L/Q334R | ++ |
| 103/104 | T194A | ++ |
| 105/106 | D37L/M316L | ++ |
| 107/108 | Q175A | ++ |
| 109/110 | G11N/T156Q/S249L/M316L | ++ |
| 111/112 | G11N/D37L | ++ |
| 113/114 | P252Q | ++ |
| 115/116 | D37L | ++ |
| 117/118 | M12F | ++ |
| 119/120 | G11N/S249L | ++ |
| 121/122 | D37L/V266L | ++ |
| 123/124 | G11N/D37L/T156Q/V266L | ++ |
| 125/126 | G11N/D37L/T156Q/K248T/V266L | ++ |
| 127/128 | D37L/I236L/K248T/S249R | ++ |
| 129/130 | D37L/T156Q/K248T/S249L | ++ |
| 131/132 | G11N/V266L/T330R/N335H | ++ |
| 133/134 | S249L | ++ |
| 135/136 | G11N/T156Q/K248T/S249R/M316L | ++ |
| 137/138 | D37L/S249L/M316L | ++ |
| 139/140 | G11N | ++ |
| 141/142 | G11N/T156Q | ++ |
| 143/144 | G11N/D37L/S249L | ++ |
| 145/146 | T330R | ++ |
| 147/148 | M316L | ++ |
| 149/150 | T156Q/S249L/V266L/N335H | ++ |
| 151/152 | S249L/V266L/M316L/T330R/N335H | + |
| 153/154 | G11N/T156Q/S249L | + |
| 155/156 | S249R | + |
| 157/158 | V266L/M316L/T330R/Q334R/N335H | + |
| 159/160 | K254V | + |
| 161/162 | T156Q/K248T/S249L/M316L/Q334R | + |
| 163/164 | G33A | + |
| 165/166 | D37L/T156Q/K248T/S249R/M316L | + |
| 167/168 | T156Q/V266L | + |
| 169/170 | K248T/S249L | + |
| 171/172 | N25M | + |
| 173/174 | T156Q/S249R/M316L/T330R/N335H | + |
| 175/176 | Y218F | + |
| 177/178 | M12V | + |
| 179/180 | M12T | + |
| 181/182 | D37L/T156Q/K248T/S249L/V266L | + |
| 183/184 | M12L | + |
| 185/186 | G11N/D37L/T156Q/K248T/S249L/M316L | + |
| 187/188 | D37L/T156Q/M316L/T330R | + |
| 189/190 | G11N/D37L/K248T/S249R/M316L/T330R | + |
| 191/192 | G11N/D37L/K248T/S249L | + |
| 193/194 | S249R/T330R/N335H | + |
| 195/196 | G11N/D37L/T156Q/T330R/Q334R | + |
| 197/198 | N25L | + |
| 199/200 | M38R | + |
| 201/202 | T40D | + |
| 203/204 | K248T/S249R | + |
| 205/206 | G11N/D37L/Y148F/S249L/M316L | + |
| 207/208 | I83V | + |
| 209/210 | S323E | + |
| 211/212 | T193Q | + |
| 213/214 | N335H | + |
| 215/216 | V257G | + |
| 217/218 | E258V | + |

Levels of increased activity were determined relative to the reference polypeptide of SEQ ID NO: 2 and defined as follows:
“+” > 1,
“++” > 1.2,
“+++” > 3.6,
“++++” > 10

### Example 6

### Improvements Over SEQ ID NO: 54 in RNA Ligase Activity

The RNA ligase of SEQ ID NO: 54 was selected as the parent enzyme in an RNA ligation assay. Libraries of engineered genes were produced using known techniques (e.g., saturation mutagenesis, recombination of previously identified beneficial mutations). The polypeptides encoded by each gene were produced in high throughput (HTP) as described in Example 1, and the cell paste was generated as described in Example 2. To prepare lysate, 600 ul of 0.1 mg/mL lysozyme in 50 mM Tris-HCl buffer at pH 7.5 was added to the cell paste of each sample. After thorough resuspension at room temperature on a plate mixer for 30 min, the cells were incubated at 40° C. for 1 hour in a PCR thermocycler. The samples were then centrifuged for 10 min at 4000 rpm and 4° C., and the clear supernatants were collected for ligation reactions.

The oligonucleotide mix for Reaction 1 in Example 4 were used as substrates in the ligation reaction. The lysate was screened in a 100 nanoliter reaction that comprised 50 mM Tris-HCl buffer at pH 7.5, 2 mM MgCl₂, 1 mM dithiothreitol (DTT), 0.4 mM adenosine triphosphate (ATP), and 1.6 mM of an equimolar mixture of the oligonucleotide fragments compromising Reaction 1. Nanoliter scale liquid transfers were carried out by Echo Acoustic Liquid Handler. The reactions were mixed by vortex, incubated for 2 hr at 37° C. in a PCR thermocycler, and quenched and diluted for analytical analysis by CE as described in Example 4. Using the peak area from CE analytical method, the % conversion for each sample was calculated as shown below:

$$\% conversion = \frac{area under the curve for the product peak}{\begin{matrix}Total area under the curve for \\ all products and remaining fragments\end{matrix}} * 100$$

Activity relative to SEQ ID NO: 54 (Activity FIOP) was calculated as the % conversion by the variant over the % conversion by SEQ ID NO: 54. The result is shown in Table 6.1.

TABLE 6.1
Activities of Variants Relative to SEQ ID NO: 54

| | | Activity |
|---|---|---|
| | Amino Acid | FIOP |
| SEQ ID | Differences | Relative to |
| NO: | (Relative to | SEQ ID |
| (nt/aa) | SEQ ID NO: 54) | NO: 54 |
| 219/220 | T156Q/T232G/Q334R/N335H | ++++ |
| 221/222 | M12V/K254V/K256A | ++++ |
| 223/224 | M12V/N230T | ++++ |
| 225/226 | M12L/N230T/K251T | ++++ |
| 227/228 | M12L/N230T/K251S/K254V/K256A | ++++ |
| 229/230 | M12A/M38R/N230T | ++++ |
| 231/232 | N230T/K251S | ++++ |
| 233/234 | M12P/M38R/K254V | ++++ |
| 235/236 | M12V/N230T/K251T | ++++ |
| 237/238 | N230T/K256A | ++++ |
| 239/240 | K254V | ++++ |
| 241/242 | N230T | ++++ |
| 243/244 | M12T/N230T/K251S | ++++ |
| 245/246 | M12T/N230T/K254V | ++++ |
| 247/248 | M12V/M38R/N230T/K251T/K256A | ++++ |
| 249/250 | M12L/K251S | ++++ |
| 251/252 | M12T/K251T/K254V/K256A | +++ |
| 253/254 | M12V/N230T/K251S | +++ |
| 255/256 | M38R | +++ |
| 257/258 | M12T/N230T/K256A | +++ |
| 259/260 | M12L | +++ |
| 261/262 | M12V | +++ |
| 263/264 | M12L/N25M/M38R/N230T/K251T | +++ |
| 265/266 | M12L/M38R/M102T/N230T/K251T/K256A | +++ |
| 267/268 | M12L/N230T | +++ |
| 269/270 | M12V/K256A | +++ |
| 271/272 | M38R/N230T/K256A | +++ |
| 273/274 | M12V/N230T/K251S/K254V/K256A | +++ |
| 275/276 | K256A | +++ |
| 277/278 | M12T/N230T/K251S/K256A | +++ |
| 279/280 | M12T/K251T | +++ |
| 281/282 | N230T/K251S/K254L | +++ |
| 283/284 | N230T/K254V | +++ |
| 285/286 | K251S/K254L | +++ |
| 287/288 | M12T/N25L/N230T | +++ |
| 289/290 | M12V/N230T/K251S/K254L/K256A | +++ |
| 291/292 | M38R/N230T | +++ |
| 293/294 | M12T/K251S | +++ |
| 295/296 | M38R/K251T | +++ |
| 297/298 | M12V/N230T/K251S/K256A | +++ |
| 299/300 | M12L/N230T/K251T/K254V/K256A | +++ |
| 301/302 | M12T/M38R/N230T/K256A | +++ |
| 303/304 | M12L/N230T/K256A | +++ |
| 305/306 | M102T | +++ |
| 307/308 | K251T | +++ |
| 309/310 | M12V/N25M/K251T/K256A | +++ |
| 311/312 | M12T/M102T/K256A | +++ |
| 313/314 | N230T/K251T | +++ |
| 315/316 | M12T/N230T/K251S/K254L/K256A | +++ |
| 317/318 | M12V/M38R/N230T/K251T/K254V/K256A | +++ |
| 319/320 | M12T/M38R/N230T | +++ |
| 321/322 | M12L/M38R/K254V | +++ |
| 323/324 | M12L/K251T/K256A | +++ |
| 325/326 | M12T/N230T | +++ |
| 327/328 | M102T/N230T/K251S/K254V/K256A | +++ |
| 329/330 | N230T/K251S/K254V | +++ |
| 331/332 | M12T/N230T/K251S/K254V/K256A | +++ |
| 333/334 | M12T/M38R/K251S | +++ |
| 335/336 | M12T/N230T/K251S/K254V | +++ |
| 337/338 | M12T/N230T/K251T | +++ |
| 339/340 | T156Q/K248T/Q334R/N335H | +++ |
| 341/342 | M12V/M38R/N230T | +++ |
| 343/344 | M12T/N25M/M38R | +++ |
| 345/346 | M12V/M102T/N230T/K251T/K254V | +++ |
| 347/348 | M12L/K256A | +++ |
| 349/350 | M12T | +++ |
| 351/352 | N230T/K251T/K254L | +++ |
| 353/354 | M12V/N25L/M38R | +++ |
| 355/356 | M12T/M38R/K251S/K254V | +++ |
| 357/358 | M12V/K251S/K254L/K256A | +++ |
| 359/360 | M12T/K251T/K254V | +++ |
| 361/362 | M12T/M102T | +++ |
| 363/364 | M12V/M38R/K251S | +++ |
| 365/366 | M12V/N230T/K251S/K254L | +++ |
| 367/368 | M12T/K251T/K256A | +++ |
| 369/370 | M38R/N230T/K251S/K254V/K256A | +++ |
| 371/372 | M12V/N230T/K251T/K256A | +++ |
| 373/374 | M12L/N25M/N230T/K251S/K256A | +++ |
| 375/376 | M12T/N230T/R311C | +++ |
| 377/378 | M12L/M38R/N230T/K251S/K254V/K256A | ++ |
| 379/380 | M12L/N230T/K251T/K254L | ++ |
| 381/382 | M12L/K254V | ++ |
| 383/384 | M38R/N230T/K251S/K254V | ++ |
| 385/386 | M12L/M38R/K251S/K256A | ++ |
| 387/388 | M12L/M38R/K251S | ++ |
| 389/390 | T156Q/Q334R/N335H | ++ |
| 391/392 | M12A/N230T | ++ |
| 393/394 | M12P/N230T | ++ |
| 395/396 | M12T/M38R/K251T | ++ |
| 397/398 | V257G/E258P/Q334R | ++ |
| 399/400 | M12L/N25L/M38R/K251T/K256A | ++ |
| 401/402 | Q334R | ++ |
| 403/404 | M12T/N25L/N230T/K254V/K256A | ++ |
| 405/406 | M12T/K251S/K256A | ++ |
| 407/408 | M38R/K251T/K256A | ++ |
| 409/410 | M12V/K251S | ++ |
| 411/412 | M12V/K251T/K254L | ++ |
| 413/414 | M12V/M38R/N230T/K251T/K254V | ++ |
| 415/416 | M12L/M38R | ++ |
| 417/418 | M12L/K251S/K254L/K256A | ++ |
| 419/420 | V257G/E258P/Q334R/N335H | ++ |
| 421/422 | M12L/M38R/K254L/K256A | ++ |
| 423/424 | M12V/K254L | ++ |
| 425/426 | M12T/K254L | ++ |
| 427/428 | M12T/M38R | ++ |
| 429/430 | T232G/K248T/V257G/E258P/Q334R/N335H | ++ |
| 431/432 | M12L/M102T | ++ |
| 433/434 | T156Q/E258P/Q334R/N335H | ++ |
| 435/436 | T156Q/L249R/Q334R/N335H | ++ |
| 437/438 | M12T/M102T/N230T/K254L | ++ |
| 439/440 | N25M | ++ |
| 441/442 | M12V/K254L/K256A | ++ |
| 443/444 | T156H/Q334R/N335H | ++ |
| 445/446 | T156Q/V257G/E258P/Q334R/N335H | ++ |
| 447/448 | M12T/M102T/N230T | ++ |
| 449/450 | T156Q/K248T/V257G/E258P/Q334R | ++ |
| 451/452 | M12T/K256A | ++ |
| 453/454 | K248T/L249R/Q334R/N335H | ++ |
| 455/456 | T232G/V257G/E258P/Q334R/N335H | ++ |
| 457/458 | M12T/M38R/K251T/K254V | ++ |
| 459/460 | Q334R/N335H | ++ |
| 461/462 | T156Q/Q334R | ++ |
| 463/464 | M12V/M38R/K254V | ++ |
| 465/466 | K251T/K254V | ++ |
| 467/468 | M12T/M38R/M102T/K256A | + |
| 469/470 | T156Q/V257G/Q334R/N335H | + |
| 471/472 | M38R/K251S/K256A | + |
| 473/474 | M12T/N140I/K251S/K254L | + |
| 475/476 | K251S/K254V/K256A | + |
| 477/478 | M12V/K251T | + |
| 479/480 | K248T/Q334R/N335H | + |
| 481/482 | M12T/K254V | + |
| 483/484 | M12V/M38R | + |
| 485/486 | M12V/K251T/K256A | + |
| 487/488 | T156Q/E258P/Q334R | + |
| 489/490 | T156Q/K248T/T330R/N335H | + |
| 491/492 | M12T/M38R/K256A | + |
| 493/494 | T232G/Q334R/N335H | + |
| 495/496 | T156Q/L249R/Q334R | + |
| 497/498 | T330R/Q334R/N335H | + |
| 499/500 | T156Q/K248T/Q334R | + |
| 501/502 | T156Q/N335H | + |
| 503/504 | V257G/Q334R | + |
| 505/506 | T156H/K248T/Q334R/N335H | + |
| 507/508 | K248T/Q334R | + |
| 509/510 | T232G/K248T/Q334R/N335H | + |
| 511/512 | T156H/K248T/Q334R | + |
| 513/514 | T156H/Q334R | + |
| 515/516 | M12T/N25M/M38R/N230T/K251S/K256A | + |
| 517/518 | M12V/K251S/K254V/K256A | + |
| 519/520 | T156Q/K248T/L249R/Q334R/N335H | + |
| 521/522 | T330R/N335H | + |
| 523/524 | K248T/V257G/Q334R/N335H | + |
| 525/526 | T232G/K248T/Q334R | + |
| 527/528 | T156Q/T232G/K248T/Q334R/N335H | + |
| 529/530 | T232G/V257G/T330R/Q334R | + |
| 531/532 | T156Q/K248T/A291T/Q334R/N335H | + |
| 533/534 | T156Q/T232G/K248T/V257G/E258P/Q334R | + |
| 535/536 | M12V/M38R/K251T/K254L | + |
| 537/538 | M12T/N25L/M38R | + |
| 539/540 | T156Q/T232G/L249R/Q334R/N335H | + |
| 541/542 | M12T/N25M | + |
| 543/544 | T156Q/K248T/T330R/Q334R/N335H | + |
| 545/546 | T156Q/K248T/L249R/Q334R | + |
| 547/548 | T156H/T232G/K248T/Q334R/N335H | + |
| 549/550 | T232G/K248T/T330R/N335H | + |
| 551/552 | T156Q/T232G/T330R/Q334R/N335H | + |

Levels of increased activity were determined relative to the reference polypeptide of SEQ ID NO: 54 and defined as follows:
“+” > 1,
“++” > 1.5,
“+++” > 2.35,
“++++” > 3.52

In another assay, lysate was screened in a 100 nanoliter reaction that comprised 50 mM Tris-HCl buffer at pH 7.5, 2 mM MgCl₂, 1 mM dithiothreitol (DTT), 1.6 mM adenosine triphosphate (ATP), and 0.8 mM of an equimolar mixture of the oligonucleotide fragments compromising Reaction 1. Nanoliter scale liquid transfers were carried out by Echo Acoustic Liquid Handler. The reactions were mixed by vortex, incubated for 2 hr at 37° C. in a PCR thermocycler, and quenched and diluted for analytical analysis by CE as described in Example 4. Using the peak area from CE analytical method, the % conversion for each sample was calculated as shown below:

$$\% conversion = \frac{area under the curve for the product peak}{\begin{matrix}Total area under the curve for \\ all products and remaining fragments\end{matrix}} * 100$$

Activity relative to SEQ ID NO: 54 (Activity FIOP) was calculated as the % conversion by the variant over the % conversion by SEQ ID NO: 54. The result is shown in Table 6.2.

TABLE 6.2
Activities of Variants Relative to SEQ ID NO: 54

| SEQ ID | Amino Acid | Activity FIOP |
|---|---|---|
| NO: | Differences | Relative to SEQ |
| (nt/aa) | (Relative to SEQ ID NO: 54) | ID NO: 54 |
| 553/554 | E197W | ++++ |
| 555/556 | R338L | ++++ |
| 557/558 | K327S | ++++ |
| 559/560 | D192F | ++++ |
| 561/562 | I331S | ++++ |
| 563/564 | K326C | ++++ |
| 565/566 | R338G | ++++ |
| 567/568 | L345S | ++++ |
| 569/570 | K327G | ++++ |
| 571/572 | A101V | +++ |
| 573/574 | N100V | +++ |
| 575/576 | I331G | +++ |
| 577/578 | E141G | +++ |
| 579/580 | H198M | +++ |
| 581/582 | K326L | +++ |
| 583/584 | V64W | +++ |
| 585/586 | I306G | +++ |
| 587/588 | L345Q | +++ |
| 589/590 | A101W | ++ |
| 591/592 | N100R | ++ |
| 593/594 | I331W | ++ |
| 595/596 | L317F | ++ |
| 597/598 | R338M | ++ |
| 599/600 | R265F | ++ |
| 601/602 | R296W | ++ |
| 603/604 | T194V | ++ |
| 605/606 | E62V | ++ |
| 607/608 | L317W | ++ |
| 609/610 | D90F | ++ |
| 611/612 | N307R | + |
| 613/614 | L317G | + |
| 615/616 | N100T | + |
| 617/618 | N100L | + |
| 619/620 | T194G | + |
| 621/622 | D103G | + |
| 623/624 | E62A | + |
| 625/626 | S210G | + |
| 627/628 | V105R | + |
| 629/630 | T61A | + |

Levels of increased activity were determined relative to the reference polypeptide of SEQ ID NO: 54 and defined as follows:
“+” > 1, “++” > 1.41, “+++” > 1.7, “++++” > 2.1

### Example 7

### Improvements Over SEQ ID NO: 244 in RNA Ligase Activity

The RNA ligase of SEQ ID NO: 244 was selected as the parent enzyme in an RNA ligation assay. Libraries of engineered genes were produced using known techniques (e.g., saturation mutagenesis, recombination of previously identified beneficial mutations). The polypeptides encoded by each gene were produced in high throughput (HTP) as described in Example 1, and the cell paste was generated as described in Example 2. To prepare lysate, 600 ul of 0.1 mg/mL lysozyme in 50 mM Tris-HCl buffer at pH 7.5 was added to the cell paste of each sample. After thorough resuspension at room temperature on a plate mixer for 30 min, the cells were incubated at 40° C. for 1 hour in a PCR thermocycler. The samples were then centrifuged for 10 min at 4000 rpm and 4° C., and the clear supernatants were collected for ligation reactions.

The oligonucleotide mix for Reaction 1 in Example 4 were used as substrates in the ligation reaction. The lysate was screened in a 100 nanoliter reaction that comprised 50 mM Tris-HCl buffer at pH 7.5, 2 mM MgCl₂, 1 mM dithiothreitol (DTT), 3.2 mM adenosine triphosphate (ATP), and 1.6 mM of an equimolar mixture of the oligonucleotide fragments compromising Reaction 1. Nanoliter scale liquid transfers were carried out by Echo Acoustic Liquid Handler. The reactions were mixed by vortex, incubated for 2 hr at 37° C. in a PCR thermocycler, and quenched and diluted for analytical analysis by CE as described in Example 4. Using the peak area from CE analytical method, the % conversion for each sample was calculated as shown below:

$$\% conversion = \frac{area under the curve for the product peak}{\begin{matrix}Total area under the curve for \\ all products and remaining fragments\end{matrix}} * 100$$

Activity relative to SEQ ID NO: 244 (Activity FIOP) was calculated as the % conversion by the variant over the % conversion by SEQ ID NO: 244. The result is shown in Table 7.1.

TABLE 7.1
Activities of Variants Relative to SEQ ID NO: 244

| SEQ ID | Amino Acid | Activity FIOP |
|---|---|---|
| NO: | Differences | Relative to SEQ |
| (nt/aa) | (Relative to SEQ ID NO: 244) | ID NO: 244 |
| 631/632 | E141G/E197W/K326L/K327S | ++++ |
| 633/634 | N100V/A101V/R338L | ++++ |
| 635/636 | E141G/H198M/K326L | ++++ |
| 637/638 | V64W/N100V/A101V | ++++ |
| 639/640 | N100V/R338L | ++++ |
| 641/642 | A101V/E141G/H198M/K326L | ++++ |
| 643/644 | A101V/H198M/K326L/K327G | ++++ |
| 645/646 | N100V/A101V/R338G | ++++ |
| 647/648 | H198M/K326L | ++++ |
| 649/650 | V64W/N100V/A101V/R338G/L345Q | ++++ |
| 651/652 | V64W/N100V/A101V/R338L/L345Q | ++++ |
| 653/654 | E197W/K326L | ++++ |
| 655/656 | H198M/K327S | ++++ |
| 657/658 | E141G/H198M/K327S | ++++ |
| 659/660 | N100V/R338G | ++++ |
| 661/662 | H198M/K326L/K327G | ++++ |
| 663/664 | E197W/K326L/K327G | ++++ |
| 665/666 | V64W/N100V/R338G | ++++ |
| 667/668 | V64W/N100V/A101V/T156S/E197W/R338L | +++ |
| 669/670 | V64W/N100V/A101V/R338G | +++ |
| 671/672 | N100V/E197W/R338L | +++ |
| 673/674 | H198M/K326L/K327S | +++ |
| 675/676 | E197W/R338L | +++ |
| 677/678 | A101V/E197W/K326L | +++ |
| 679/680 | V64W/N100V/E197W/R338L | +++ |
| 681/682 | A101V/H198M | +++ |
| 683/684 | E141G/E197W/K327S | +++ |
| 685/686 | H198M/K327G | +++ |
| 687/688 | H198M/K326C/K327S | +++ |
| 689/690 | V64W/N100V/A101V/E197W/R338G | ++ |
| 691/692 | A101V/K326L | ++ |
| 693/694 | A101V/E197W/K326L/K327G | ++ |
| 695/696 | E141G/E197W/K326L/K327G | ++ |
| 697/698 | N100V/A101W/R338G | ++ |
| 699/700 | E197W/K327S | ++ |
| 701/702 | E197W | ++ |
| 703/704 | E141G/E197W | ++ |
| 705/706 | A101V | ++ |
| 707/708 | A101V/H198M/K326C | ++ |
| 709/710 | V64W/E197W/H198M/R338G | ++ |
| 711/712 | E141G | ++ |
| 713/714 | V64W/N100V/A101W/R338G | ++ |
| 715/716 | A101V/E141G | ++ |
| 717/718 | N100V/A101V/E197W/R338L | ++ |
| 719/720 | E141G/K326C | ++ |
| 721/722 | R338L | ++ |
| 723/724 | E197W/R338G | ++ |
| 725/726 | K326L | ++ |
| 727/728 | E197W/H198M/R338G | ++ |
| 729/730 | E197W/H198M/R338L | ++ |
| 731/732 | V64W/A101W/R338L | ++ |
| 733/734 | V64W/E197W/H198M | ++ |
| 735/736 | A101V/E197W | ++ |
| 737/738 | N100V/A101V | ++ |
| 739/740 | E141G/K326L | ++ |
| 741/742 | N100V/I109F/E197W/R338L | ++ |
| 743/744 | A101W/E197W/R338L | ++ |
| 745/746 | V64W/N100V/A101W | ++ |
| 747/748 | E197W/H198M | ++ |
| 749/750 | N100V/A101W/R338L | ++ |
| 751/752 | E141G/K326C/K327S | ++ |
| 753/754 | N100V/E197W | ++ |
| 755/756 | E197W/K326C/K327G | ++ |
| 757/758 | V64W/N100V | ++ |
| 759/760 | A101V/K326C | ++ |
| 761/762 | N100V/A101W | + |
| 763/764 | V64W/E197W/R338L | + |
| 765/766 | N100V/E197W/H198M | + |
| 767/768 | A101W | + |
| 769/770 | A101W/R338L | + |
| 771/772 | E141G/K327S | + |
| 773/774 | A101V/I306G | + |
| 775/776 | V64W/N100V/A101W/E197W | + |
| 777/778 | A101V/K326L/K327G | + |
| 779/780 | N100V | + |
| 781/782 | K327G | + |
| 783/784 | K326C/L345S | + |
| 785/786 | V64W/N100V/A101V/E197W/H198M | + |
| 787/788 | K326C/K327S | + |
| 789/790 | V64W/A101W | + |
| 791/792 | N100V/A101V/E197W | + |
| 793/794 | A101V/K327S | + |
| 795/796 | R338G | + |
| 797/798 | N100V/A101V/E197W/R338G | + |
| 799/800 | N100V/A101W/E197W/R338G | + |
| 801/802 | V64W/N100V/E197W/H198M/R338G | + |
| 803/804 | I306G | + |
| 805/806 | V64W/R338G | + |
| 807/808 | V64W/N100V/A101V/E197W | + |
| 809/810 | V64W/E197W/H198M/R338L | + |
| 811/812 | A101V/K327G | + |
| 813/814 | A101V/E141G/K326C/K327S | + |
| 815/816 | V64W/A101W/E197W/R338L | + |
| 817/818 | A101W/R338G | + |
| 819/820 | A101V/H198M/I306G/K327S | + |

Levels of increased activity were determined relative to the reference polypeptide of SEQ ID NO: 244 and defined as follows
“+” > 1, “++” > 2, “+++” > 7.3, “++++” > 16

### Example 8

### Improvements Over SEQ ID NO: 648 in RNA Ligase Activity

The RNA ligase of SEQ ID NO: 648 was selected as the parent enzyme in an RNA ligation assay. Libraries of engineered genes were produced using known techniques (e.g., saturation mutagenesis, recombination of previously identified beneficial mutations). The polypeptides encoded by each gene were produced in high throughput (HTP) as described in Example 1, and the cell paste was generated as described in Example 2. To prepare lysate, 600 ul of 0.1 mg/mL lysozyme in 50 mM Tris-HCl buffer at pH 7.5 was added to the cell paste of each sample. After thorough resuspension at room temperature on a plate mixer for 30 min, the cells were diluted 4-fold in 50 mM Tris-HCl buffer at pH 7.5 then incubated at 40° C. for 1 hour in a PCR thermocycler. The samples were then centrifuged for 10 min at 4000 rpm and 4° C., and the clear supernatants were collected for ligation reactions.

The oligonucleotide mix for Reaction 1 in Example 4 were used as substrates in the ligation reaction. The lysate was screened in a 100 nanoliter reaction that comprised 50 mM Tris-HCl buffer at pH 7.5, 2 mM MgCl₂, 1 mM dithiothreitol (DTT), 4 mM adenosine triphosphate (ATP), and 1.6 mM of an equimolar mixture of the oligonucleotide fragments compromising Reaction 1. Nanoliter scale liquid transfers were carried out by Echo Acoustic Liquid Handler. The reactions were mixed by vortex, incubated for 2 hr at 37° C. in a PCR thermocycler, and quenched and diluted for analytical analysis by CE as described in Example 4. Using the peak area from CE analytical method, the % conversion for each sample was calculated as shown below:

$$\% conversion = \frac{area under the curve for the product peak}{\begin{matrix}Total area under the curve for \\ all products and remaining fragments\end{matrix}} * 100$$

Activity relative to SEQ ID NO: 648 (Activity FIOP) was calculated as the % conversion by the variant over the % conversion by SEQ ID NO: 648. The result is shown in Table 8.1.

TABLE 8.1
Activities of Variants Relative to SEQ ID NO: 648

| SEQ | Amino Acid | Activity FIOP |
|---|---|---|
| ID NO: | Differences | Relative to SEQ |
| (nt/aa) | (Relative to SEQ ID NO: 648) | ID NO: 648 |
| 821/822 | T194V/T330D/I331W | ++++ |
| 823/824 | M298S | ++++ |
| 825/826 | I331W | ++++ |
| 827/828 | D192L/T193K/T194V/M298S | ++++ |
| 829/830 | T330D | ++++ |
| 831/832 | D192L/T330D/I331G | ++++ |
| 833/834 | D192L/T193K/T194V/I331S | ++++ |
| 835/836 | M298I/T330D/I331S | ++++ |
| 837/838 | T194V/M298S/T330D/I331G | +++ |
| 839/840 | D192L/T193K/T330D/I331S | +++ |
| 841/842 | D192L/M298S/I331S | +++ |
| 843/844 | D192L/T193K | +++ |
| 845/846 | T194V/T330D | +++ |
| 847/848 | I331G | +++ |
| 849/850 | D192L/M298S/I331W | ++ |
| 851/852 | P60D/N100R/T156G | ++ |
| 853/854 | T193K/R296G/I331W | ++ |
| 855/856 | M298S/I331G | ++ |
| 857/858 | T193K/T194V/T330D/I331W | ++ |
| 859/860 | T194V | ++ |
| 861/862 | T194V/M298S/I331G | ++ |
| 863/864 | T193K/M298S | ++ |
| 865/866 | T193K/T330D | ++ |
| 867/868 | D192L | ++ |
| 869/870 | D192L/T193K/T194V/M298S/I331S | ++ |
| 871/872 | D192L/T193K/T194V/M298S/T330D | ++ |
| 873/874 | D192L/T193K/M298S/I331S | ++ |
| 875/876 | N100R/T156G | ++ |
| 877/878 | M298S/I331S | ++ |
| 879/880 | M298I/I331G | ++ |
| 881/882 | E62V/N100V | ++ |
| 883/884 | M298S/T330D | + |
| 885/886 | T193K/M298I | + |
| 887/888 | I331S | + |
| 889/890 | P60D/N100R | + |
| 891/892 | D192L/T193K/M298S/T330D/I331S | + |
| 893/894 | D192L/T193K/T194V/M298S/I331W | + |
| 895/896 | D192L/T330D/I331S | + |
| 897/898 | N100R | + |
| 899/900 | T193K/T194V/M298S | + |
| 901/902 | T194V/M298S/I331S | + |
| 903/904 | R338L | + |
| 905/906 | E62V | + |

Levels of increased activity were determined relative to the reference polypeptide of SEQ ID NO: 648 and defined as follows:
“+” > 1, “++” > 1.5, “+++” > 2.5, “++++” > 4.5

In another assay, cell pastes were lysed by the addition of 600 ul of 0.1 mg/mL lysozyme in 50 mM Tris-HCl buffer at pH 7.5 to each sample. After thorough resuspension at room temperature on a plate mixer for 30 min, lysates were diluted 8-fold in 50 mM Tris-HCL buffer at pH 7.5 then were incubated at 40° C. for 1 hour in a PCR thermocycler. The samples were then centrifuged for 10 min at 4000 rpm and 4° C., and the clear supernatants were collected for ligation reactions. Lysate was diluted 8-fold in 50 mM Tris-HCl buffer at pH 7.5 and screened in a 100 nanoliter reaction that comprised 50 mM Tris-HCl buffer at pH 7.5, 20 mM MgCl₂, 1 mM dithiothreitol (DTT), 8 mM adenosine triphosphate (ATP), and 1.6 mM of an equimolar mixture of the oligonucleotide fragments compromising Reaction 1. Nanoliter scale liquid transfers were carried out by Echo Acoustic Liquid Handler. The reactions were mixed by vortex, incubated for 2 hr at 37° C. in a PCR thermocycler, and quenched and diluted for analytical analysis by CE as described in Example 4. Using the peak area from CE analytical method, the % conversion for each sample was calculated as shown below:

$$\% conversion = \frac{area under the curve for the product peak}{\begin{matrix}Total area under the curve for \\ all products and remaining fragments\end{matrix}} * 100$$

Activity relative to SEQ ID NO: 648 (Activity FIOP) was calculated as the % conversion by the variant over the % conversion by SEQ ID NO: 648. The result is shown in Table 8.2.

TABLE 8.2
Activities of Variants Relative to SEQ ID NO: 648

| SEQ ID | Amino Acid | Activity FIOP |
|---|---|---|
| NO: | Differences | Relative to SEQ |
| (nt/aa) | (Relative to SEQ ID NO: 648) | ID NO: 648 |
| 907/908 | A247S | ++++ |
| 909/910 | S18H | ++++ |
| 911/912 | L37A | ++++ |
| 913/914 | K254S | +++ |
| 915/916 | G33L | +++ |
| 917/918 | E261T | +++ |
| 919/920 | S251Y | +++ |
| 921/922 | T260G | +++ |
| 923/924 | S251C | ++ |
| 925/926 | T12R | ++ |
| 927/928 | G117A | ++ |
| 929/930 | K256S | ++ |
| 931/932 | A255Y | ++ |
| 933/934 | A255S | + |
| 935/936 | M38E | + |
| 937/938 | T12M | + |
| 939/940 | S251T | + |
| 941/942 | A255V | + |
| 943/944 | E29D | + |
| 945/946 | A247R | + |
| 947/948 | N25A | + |
| 949/950 | K256E | + |
| 951/952 | P252A | + |
| 953/954 | E261D | + |

Levels of increased activity were determined relative to the reference polypeptide of SEQ ID NO: 648 and defined as follows:
“+” > 1, “++” > 1.1, “+++” > 1.31, “++++” > 1.8

### Example 9

### Improvements Over SEQ ID NO: 862 in RNA Ligase Activity

The RNA ligase of SEQ ID NO: 862 was selected as the parent enzyme in an RNA ligation assay. Libraries of engineered genes were produced using known techniques (e.g., saturation mutagenesis, recombination of previously identified beneficial mutations). The polypeptides encoded by each gene were produced in high throughput (HTP) as described in Example 1, and the cell paste was generated as described in Example 2. To prepare lysate, 600 ul of 0.1 mg/mL lysozyme in 50 mM Tris-HCl buffer at pH 7.5 was added to the cell paste of each sample. After thorough resuspension at room temperature on a plate mixer for 30 min, the cells were diluted 8-fold in 50 mM Tris-HCl buffer at pH 7.5 then incubated at 40° C. for 1 hour in a PCR thermocycler. The samples were then centrifuged for 10 min at 4000 rpm and 4° C., and the clear supernatants were collected for ligation reactions.

The oligonucleotide mix for Reaction 1 in Example 4 were used as substrates in the ligation reaction. The lysate was screened in a 100 nanoliter reaction that comprised 50 mM Tris-HCl buffer at pH 7.5, 20 mM MgCl₂, 1 mM dithiothreitol (DTT), 8 mM adenosine triphosphate (ATP), and 1.6 mM of an equimolar mixture of the oligonucleotide fragments compromising Reaction 1. Nanoliter scale liquid transfers were carried out by Echo Acoustic Liquid Handler. The reactions were mixed by vortex, incubated for 2 hr at 37° C. in a PCR thermocycler, and quenched and diluted for analytical analysis by CE as described in Example 4. Using the peak area from CE analytical method, the % conversion for each sample was calculated as shown below:

$$\% conversion = \frac{area under the curve for the product peak}{\begin{matrix}Total area under the curve for \\ all products and remaining fragments\end{matrix}} * 100$$

Activity relative to SEQ ID NO: 862 (Activity FIOP) was calculated as the % conversion by the variant over the % conversion by SEQ ID NO: 862. The result is shown in Table 9.1.

TABLE 9.1
Activities of Variants Relative to SEQ ID NO: 862

| SEQ ID | Amino Acid | Activity FIOP |
|---|---|---|
| NO: | Differences | Relative to SEQ |
| (nt/aa) | (Relative to SEQ ID NO: 862) | ID NO: 862 |
| 955/956 | K254S | ++++ |
| 957/958 | S18H/K254S | ++++ |
| 959/960 | A247S/L317W | ++++ |
| 961/962 | L37A/L317W/H342R | ++++ |
| 963/964 | G33L | ++++ |
| 965/966 | A247S/L317F | ++++ |
| 967/968 | S18H/T156G/K254S | ++++ |
| 969/970 | S18H | ++++ |
| 971/972 | T156G | +++ |
| 973/974 | K254S/R338L | +++ |
| 975/976 | K254S/N307R | +++ |
| 977/978 | G33L/L37A/L317W | +++ |
| 979/980 | L37A | +++ |
| 981/982 | L37A/T156G/L317W | +++ |
| 983/984 | T156G/R265F/L317W | +++ |
| 985/986 | R265F/L317W | +++ |
| 987/988 | T156G/K254S | +++ |
| 989/990 | G33L/T156G/L317W | +++ |
| 991/992 | L317F | +++ |
| 993/994 | T156G/L317F | +++ |
| 995/996 | L317W | +++ |
| 997/998 | T156G/L317W | +++ |
| 999/1000 | L37A/L317W | +++ |
| 1001/1002 | S18H/R338M | +++ |
| 1003/1004 | R338M | +++ |
| 1005/1006 | T156G/A247S/L317W | +++ |
| 1007/1008 | S18H/R338L | +++ |
| 1009/1010 | G33L/L317W | +++ |
| 1011/1012 | L37A/A247S/R265F/L317F | +++ |
| 1013/1014 | L37A/L317F | ++ |
| 1015/1016 | G33L/L37A/T156G/L317W | ++ |
| 1017/1018 | T156G/A247S/R296W/L317W | ++ |
| 1019/1020 | S18H/N307R | ++ |
| 1021/1022 | T156G/R296W/L317W | ++ |
| 1023/1024 | T156G/A247S | ++ |
| 1025/1026 | L37A/A247S/L317F | ++ |
| 1027/1028 | T156G/R265F/L317F | ++ |
| 1029/1030 | S18H/K254S/N307R | ++ |
| 1031/1032 | N307R | ++ |
| 1033/1034 | R265F | ++ |
| 1035/1036 | R265F/L317F | + |
| 1037/1038 | G33L/L37A | + |
| 1039/1040 | S18H/T156G | + |
| 1041/1042 | G33L/T156G/R296W/L317W | + |
| 1043/1044 | S18H/T156G/K254S/R338L | + |
| 1045/1046 | L37A/R265F/L317F | + |
| 1047/1048 | G33L/L37A/T156G/A247S/L317W | + |
| 1049/1050 | R296W | + |
| 1051/1052 | G33L/R296W/L317W | + |
| 1053/1054 | T156G/A247S/L317F | + |
| 1055/1056 | T156G/R296W | + |
| 1057/1058 | R338L | + |
| 1059/1060 | L37A/R265F/L317W | + |
| 1061/1062 | A247S/R296W/L317F | + |
| 1063/1064 | G33L/T156G/L317F | + |
| 1065/1066 | L37A/R296W/L317F | + |
| 1067/1068 | A247S/R296W | + |
| 1069/1070 | G33L/L37A/T156G | + |
| 1071/1072 | R296W/L317W | + |
| 1073/1074 | T156G/K254S/N307R | + |
| 1075/1076 | S18H/T156G/N307R | + |
| 1077/1078 | G33L/L37A/R265F | + |

Levels of increased activity were determined relative to the reference polypeptide of SEQ ID NO: 862 and defined as follows:
“+” > 1, “++” > 1.3, “+++” > 1.48, “++++” > 1.8

### Example 10

### Improvements Over SEQ ID NO: 956 in RNA Ligase Activity

The RNA ligase of SEQ ID NO: 956 was selected as the parent enzyme in an RNA ligation assay. Libraries of engineered genes were produced using known techniques (e.g., saturation mutagenesis, recombination of previously identified beneficial mutations). The polypeptides encoded by each gene were produced in high throughput (HTP) as described in Example 1, and the cell paste was generated as described in Example 2. To prepare lysate, 600 ul of 0.1 mg/mL lysozyme in 50 mM Tris-HCl buffer at pH 7.5 was added to the cell paste of each sample. After thorough resuspension at room temperature on a plate mixer for 30 min, the cells were diluted 16-fold in 50 mM Tris-HCl buffer at pH 7.5 then incubated at 40° C. for 1 hour in a PCR thermocycler. The samples were then centrifuged for 10 min at 4000 rpm and 4° C., and the clear supernatants were collected for ligation reactions.

The oligonucleotide mix for Reaction 1 in Example 4 were used as substrates in the ligation reaction. The lysate was screened in a 100 nanoliter reaction that comprised 50 mM Tris-HCl buffer at pH 7.5, 20 mM MgCl₂, 1 mM dithiothreitol (DTT), 8 mM adenosine triphosphate (ATP), and 1.6 mM of an equimolar mixture of the oligonucleotide fragments compromising Reaction 1. Nanoliter scale liquid transfers were carried out by Echo Acoustic Liquid Handler. The reactions were mixed by vortex, incubated for 2 hr at 37° C. in a PCR thermocycler, and quenched and diluted for analytical analysis by CE as described in Example 4. Using the peak area from CE analytical method, the % conversion for each sample was calculated as shown below:

$$\% conversion = \frac{area under the curve for the product peak}{\begin{matrix}Total area under the curve for \\ all products and remaining fragments\end{matrix}} * 100$$

Activity relative to SEQ ID NO: 956 (Activity FIOP) was calculated as the % conversion by the variant over the % conversion by SEQ ID NO: 956. The result is shown in Table 10.1.

TABLE 10.1
Activities of Variants Relative to SEQ ID NO: 956

| SEQ ID | Amino Acid | Activity FIOP |
|---|---|---|
| NO: | Differences | Relative to SEQ |
| (nt/aa) | (Relative to SEQ ID NO: 956) | ID NO: 956 |
| 1079/1080 | T12M/N25A/E29D/T156G | ++++ |
| 1081/1082 | T12M/A255Y/K256S/V257D | ++++ |
| 1083/1084 | T12M/T156G/A255V | ++++ |
| 1085/1086 | A247R/P252A | ++++ |
| 1087/1088 | T12M/N25A | ++++ |
| 1089/1090 | T12M | ++++ |
| 1091/1092 | T12M/N25A/E29D/T156G/A255S | ++++ |
| 1093/1094 | K256S | ++++ |
| 1095/1096 | T12M/T156G | ++++ |
| 1097/1098 | T12M/N25A/T156G/A255V/K256S/V257D/E261D | ++++ |
| 1099/1100 | S18H/A247R/S251T | ++++ |
| 1101/1102 | N25A/A255V/K256S/V257D | ++++ |
| 1103/1104 | T12M/N25A/V257D | ++++ |
| 1105/1106 | A255S/V257D | ++++ |
| 1107/1108 | T12M/N25A/E29D/A255Y/V257D | ++++ |
| 1109/1110 | K256S/V257D | ++++ |
| 1111/1112 | T12M/A255S/V257D | ++++ |
| 1113/1114 | T12M/T156G/A255N/K256S/V257D | ++++ |
| 1115/1116 | N25A | ++++ |
| 1117/1118 | S18H/M38E/S251T/P252A | ++++ |
| 1119/1120 | T12M/K256S/V257D/E261D | ++++ |
| 1121/1122 | T12M/E29D | +++ |
| 1123/1124 | T12M/N25A/A255V/K256S/V257D/E261D | +++ |
| 1125/1126 | M38E | +++ |
| 1127/1128 | T12M/N25A/E29D/K256S/V257D | +++ |
| 1129/1130 | E29D/V257D | +++ |
| 1131/1132 | T12M/E261D | +++ |
| 1133/1134 | S251T | +++ |
| 1135/1136 | T12M/T156G/A255N | +++ |
| 1137/1138 | L249R/S251T | +++ |
| 1139/1140 | T12M/A255V | +++ |
| 1141/1142 | T12M/E29D/T156G/K256S/V257D | +++ |
| 1143/1144 | P252A | +++ |
| 1145/1146 | A255V/V257D/E261D | +++ |
| 1147/1148 | S18A | +++ |
| 1149/1150 | E29D/A255N/V257D | +++ |
| 1151/1152 | A255Y/K256S/V257D | +++ |
| 1153/1154 | T12M/N25A/E29D/T156G/K256S/V257D | +++ |
| 1155/1156 | N25A/A255V/K256S/V257D/E261D | +++ |
| 1157/1158 | E29D/A255Y/K256S/V257D/E261D | +++ |
| 1159/1160 | N25A/K256S | +++ |
| 1161/1162 | S18H/M38E/A247R/P252A/K327M | +++ |
| 1163/1164 | S18H/A247R | +++ |
| 1165/1166 | N25A/E29D | +++ |
| 1167/1168 | T12M/N25A/T156G/A255V/V257D | +++ |
| 1169/1170 | A255V/V257D | +++ |
| 1171/1172 | T12M/A255N | +++ |
| 1173/1174 | S18H/T156G/S251T/P252A | +++ |
| 1175/1176 | T12M/A255V/E261D | ++ |
| 1177/1178 | N25A/A255V | ++ |
| 1179/1180 | T12M/A255V/K256S/E261D | ++ |
| 1181/1182 | T156G/A255S/V257D | ++ |
| 1183/1184 | T156G/V257D | ++ |
| 1185/1186 | T12M/T156G/K256S/V257D | ++ |
| 1187/1188 | T12M/T156G/A255Y | ++ |
| 1189/1190 | N25A/T156G/K256S/V257D | ++ |
| 1191/1192 | T12M/N25A/E29D/T156G/V257D/E261D | ++ |
| 1193/1194 | N25A/E261D | ++ |
| 1195/1196 | T12M/T156G/A255Y/K256S/E261D | ++ |
| 1197/1198 | T12M/N25A/E29D/A255Y/K256S/V257D | ++ |
| 1199/1200 | M38E/P252A | ++ |
| 1201/1202 | T12M/T156G/A255V/K256S/V257D | ++ |
| 1203/1204 | A255S | ++ |
| 1205/1206 | L249R/P252A | ++ |
| 1207/1208 | T12M/T156G/A255Y/K256S | ++ |
| 1209/1210 | T12M/N25A/E29D/E261D | ++ |
| 1211/1212 | V257D | ++ |
| 1213/1214 | T12M/A255N/K256S | ++ |
| 1215/1216 | T12M/E29D/A255N | ++ |
| 1217/1218 | T12M/A255V/V257D/E261D | ++ |
| 1219/1220 | T12M/A255Y/V257D | ++ |
| 1221/1222 | T12M/T156G/V257D | ++ |
| 1223/1224 | D192N/L249R/S251T/P252A | ++ |
| 1225/1226 | T12M/N25A/E29D/A255N/K256S/V257D | ++ |
| 1227/1228 | T12M/N25A/E29D/A255V/E261D | ++ |
| 1229/1230 | T12M/T156G/A255Y/V257D | ++ |
| 1231/1232 | T12M/E29D/A255V/V257D | ++ |
| 1233/1234 | T12M/E29D/K256S/V257D | ++ |
| 1235/1236 | S18A/P252A | ++ |
| 1237/1238 | L249R | ++ |
| 1239/1240 | T156G/D250I | ++ |
| 1241/1242 | M38E/S251T/P252A | ++ |
| 1243/1244 | T156G/A255Y/K256S/V257D | ++ |
| 1245/1246 | N25A/V257D | ++ |
| 1247/1248 | T12M/A255V/K256S/V257D/E261D | ++ |
| 1249/1250 | N25A/T156G/E261D | ++ |
| 1251/1252 | T156G/P252A | ++ |
| 1253/1254 | T12M/N25A/A255V/K256S/V257D | + |
| 1255/1256 | T12M/N25A/E29D/T156G/A255S/V257D | + |
| 1257/1258 | E29D/T156G | + |
| 1259/1260 | S251T/P252A | + |
| 1261/1262 | K256S/V257D/E261D | + |
| 1263/1264 | N25A/T156G/A255Y/K256S/V257D | + |
| 1265/1266 | T12M/T156G/A255Y/K256S/V257D | + |
| 1267/1268 | T156G/L249R/S251T/P252A | + |
| 1269/1270 | N25A/E29D/A255V | + |
| 1271/1272 | T12M/E29D/T156G | + |
| 1273/1274 | A255V/K256S | + |
| 1275/1276 | T12M/A255Y/K256S | + |
| 1277/1278 | M38E/D250I/P252A | + |
| 1279/1280 | A247R/L249R/P252A | + |
| 1281/1282 | T12M/K256S/V257D | + |
| 1283/1284 | T12M/T156G/A255S/V257D | + |
| 1285/1286 | T12M/T156G/A255V/V257D | + |
| 1287/1288 | P60S | + |
| 1289/1290 | T12M/A255Y/K256S/E261D | + |
| 1291/1292 | T12M/K256S | + |
| 1293/1294 | T12M/A255S | + |
| 1295/1296 | A255S/V257D/E261D | + |
| 1297/1298 | T12M/T156G/K256S | + |
| 1299/1300 | T12M/A255Y/V257D/E261D | + |
| 1301/1302 | N25A/A255V/V257D/E261D | + |
| 1303/1304 | T12M/N25A/E29D/A255N/K256S/E261D | + |
| 1305/1306 | T12M/A255V/V257D | + |
| 1307/1308 | T12M/E29D/T156G/A255V/K256S | + |
| 1309/1310 | A255S/K256S | + |
| 1311/1312 | T12M/N25A/T156G/A255V/K256S | + |
| 1313/1314 | A247R/S251T/P252A | + |
| 1315/1316 | K256S/E261D | + |
| 1317/1318 | M38E/S251T | + |
| 1319/1320 | T12M/N25A/E29D/T156G/E261D | + |
| 1321/1322 | T12M/V257D | + |
| 1323/1324 | T156G/A255N/K256S/E261D | + |
| 1325/1326 | T12M/N25A/T156G/A255S | + |
| 1327/1328 | N25A/A255S | + |
| 1329/1330 | T12M/N25A/E29D/T156G/A255V/V257D/E261D | + |
| 1331/1332 | T12M/E29D/K256S/E261D | + |
| 1333/1334 | T156G/A255V/V257D | + |
| 1335/1336 | E29D/T156G/E261D | + |
| 1337/1338 | T12M/A255S/V257D/E261D | + |
| 1339/1340 | N25A/E29D/T156G/V257D/E261D | + |
| 1341/1342 | T12M/N25A/A255V/K256S/E261D | + |
| 1343/1344 | T12M/T156G/A255V/E261D | + |

Levels of increased activity were determined relative to the reference polypeptide of SEQ ID NO: 956 and defined as follows:
“+” > 1, “++” > 1.3, “+++” > 1.6, “++++” > 2.1

### Example 11

### Improvements Over SEQ ID NO: 956 in RNA Ligase Thermostability

The RNA ligase of SEQ ID NO: 956 was selected as the parent enzyme in an RNA ligation assay. Libraries of engineered genes were produced using known techniques (e.g., saturation mutagenesis, recombination of previously identified beneficial mutations). The polypeptides encoded by each gene were produced in high throughput (HTP) as described in Example 1, and the cell paste was generated as described in Example 2. To prepare lysate, 600 ul of 0.1 mg/mL lysozyme in 50 mM Tris-HCl buffer at pH 7.5 was added to the cell paste of each sample. After thorough resuspension at room temperature on a plate mixer for 30 min, the cells were diluted 8-fold in 50 mM Tris-HCl buffer at pH 7.5 then preincubated at 48° C. for 1 hour in a PCR thermocycler. The samples were then centrifuged for 10 min at 4000 rpm and 4° C. to remove the precipitated proteins, and the remainder clear supernatants were collected for ligation reactions.

The oligonucleotide mix for Reaction 1 in Example 4 were used as substrates in the ligation reaction. The lysate was screened in a 100 nanoliter reaction that comprised 50 mM Tris-HCl buffer at pH 7.5, 20 mM MgCl₂, 1 mM dithiothreitol (DTT), 8 mM adenosine triphosphate (ATP), and 1.6 mM of an equimolar mixture of the oligonucleotide fragments compromising Reaction 1. Nanoliter scale liquid transfers were carried out by Echo Acoustic Liquid Handler. The reactions were mixed by vortex, incubated for 2 hr at 37° C. in a PCR thermocycler, and quenched and diluted for analytical analysis by CE as described in Example 4. Using the peak area from CE analytical method, the % conversion for each sample was calculated as shown below:

$$\% conversion = \frac{area under the curve for the product peak}{\begin{matrix}Total area under the curve for \\ all products and remaining fragments\end{matrix}} * 100$$

Activity relative to SEQ ID NO: 956 (Activity FIOP) was calculated as the % conversion by the variant over the % conversion by SEQ ID NO: 956. The result is shown in Table 11.1.

TABLE 11.1
Activities of Variants Relative to SEQ ID NO:
956 after 48° C. Heat Challenge for 60 min

| | Amino Acid | |
|---|---|---|
| | Differences | Activity FIOP |
| SEQ ID | (Relative to | Relative to |
| NO: (nt/aa) | SEQ ID NO: 956) | SEQ ID NO: 956 |
| 1345/1346 | L157V | ++++ |
| 1347/1348 | I183L | ++++ |
| 1349/1350 | V95A | ++++ |
| 1351/1352 | I183V | ++++ |
| 1353/1354 | N185P | +++ |
| 1355/1356 | K181E | +++ |
| 1357/1358 | G213D | +++ |
| 1359/1360 | K89M | +++ |
| 1361/1362 | K89G | ++ |
| 1363/1364 | I28M | ++ |
| 1365/1366 | L190M | ++ |
| 1367/1368 | V214L | ++ |
| 1369/1370 | V143A | + |
| 1371/1372 | K89Q | + |
| 1373/1374 | V95G | + |

Levels of increased activity after preincubation at 48° C. for 1 hour were determined relative to the reference polypeptide of SEQ ID NO: 956 and defined as follows:
“+” > 1, “++” > 1.4, “+++” > 2, “++++” > 3

### Example 12

### Improvements Over SEQ ID NO: 1088 in RNA Ligase Thermostability

The RNA ligase of SEQ ID NO: 1088 was selected as the parent enzyme in an RNA ligation assay. Libraries of engineered genes were produced using known techniques (e.g., saturation mutagenesis, recombination of previously identified beneficial mutations). The polypeptides encoded by each gene were produced in high throughput (HTP) as described in Example 1, and the cell paste was generated as described in Example 2. To prepare lysate, 600 ul of 0.1 mg/mL lysozyme in 50 mM Tris-HCl buffer at pH 7.5 was added to the cell paste of each sample. After thorough resuspension at room temperature on a plate mixer for 30 min, the cells were diluted 8-fold in 50 mM Tris-HCl buffer at pH 7.5 then preincubated at 48° C. for 1 hour in a PCR thermocycler. The samples were then centrifuged for 10 min at 4000 rpm and 4° C. to remove the precipitated proteins, and the remainder clear supernatants were collected for ligation reactions.

The oligonucleotide mix for Reaction 1 in Example 4 were used as substrates in the ligation reaction. The lysate was screened in a 100 nanoliter reaction that comprised 50 mM Tris-HCl buffer at pH 7.5, 20 mM MgCl₂, 1 mM dithiothreitol (DTT), 8 mM adenosine triphosphate (ATP), and 1.6 mM of an equimolar mixture of the oligonucleotide fragments compromising Reaction 1. Nanoliter scale liquid transfers were carried out by Echo Acoustic Liquid Handler. The reactions were mixed by vortex, incubated for 2 hr at 37° C. in a PCR thermocycler, and quenched and diluted for analytical analysis by CE as described in Example 4. Using the peak area from CE analytical method, the % conversion for each sample was calculated as shown below:

$$\% conversion = \frac{area under the curve for the product peak}{\begin{matrix}Total area under the curve for all \\ products and remaining fragments\end{matrix}} * 100$$

Activity relative to SEQ ID NO: 1088 (Activity FIOP) was calculated as the % conversion by the variant over the % conversion by SEQ ID NO: 1088. The result is shown in Table 12.1.

TABLE 12.1
Activities of Variants Relative to SEQ ID NO:
1088 after 48° C. Heat Challenge for 60 min

| | Amino Acid | Activity FIOP |
|---|---|---|
| SEQ ID | Differences | Relative to |
| NO: (nt/aa) | (Relative to SEQ ID NO: 1088) | SEQ ID NO: 1088 |
| 1375/1376 | V95A/L157V | ++++ |
| 1377/1378 | V95G/I183V/N185P/V214L | ++++ |
| 1379/1380 | K89M/I183V | ++++ |
| 1381/1382 | I28M/K89G/V143M/I183L | ++++ |
| 1383/1384 | I28M/V143M/L157V | +++ |
| 1385/1386 | K181E/I183V | +++ |
| 1387/1388 | V143A/K181E/I183V/V214L | +++ |
| 1389/1390 | V143A/K181E/I183V | +++ |
| 1391/1392 | K89G/V143M/I183L | +++ |
| 1393/1394 | I28M/V143M/K181E/I183L | +++ |
| 1395/1396 | V143M/L157V | +++ |
| 1397/1398 | V95A/K181E/I183L | ++ |
| 1399/1400 | L157V | ++ |
| 1401/1402 | V143A/I183L | ++ |
| 1403/1404 | V143A/L157V | ++ |
| 1405/1406 | K89G/V143A/L157V/I183L | ++ |
| 1407/1408 | K89M/V95A/L157V | ++ |
| 1409/1410 | K89M/L157V | ++ |
| 1411/1412 | K89G/V95A/V143A/K181E | ++ |
| 1413/1414 | V95A/V143A | ++ |
| 1415/1416 | K89M/L157V/I183L/V214L | + |
| 1417/1418 | V143M/V214L | + |
| 1419/1420 | V143M/K181E/I183V/N185P | + |
| 1421/1422 | I28M/V95A/V143A | + |
| 1423/1424 | V143M/I183L/V214L | + |
| 1425/1426 | I28M/V95A/I183V/V214L | + |
| 1427/1428 | K89M/V143A/L157V | + |
| 1429/1430 | I28M/V95A | + |
| 1431/1432 | V95A/V143M/I183V/N185P | + |
| 1433/1434 | V95A/V143A/L157V/I183L | + |

Levels of increased activity after preincubation at 48° C. for 1 hour were determined relative to the reference polypeptide of SEQ ID NO: 956 and defined as follows:
“+” > 1, “++” > 3, “+++” > 5, “++++” > 9

### Example 13

### Improvements Over SEQ ID NO: 1416 in RNA Ligase Activity

The RNA ligase of SEQ ID NO: 1416 was selected as the parent enzyme in an RNA ligation assay. Libraries of engineered genes were produced using known techniques (e.g., saturation mutagenesis, recombination of previously identified beneficial mutations). The polypeptides encoded by each gene were produced in high throughput (HTP) as described in Example 1, and the cell paste was generated as described in Example 2. To prepare lysate, 600 ul of 0.1 mg/mL lysozyme in 50 mM Tris-HCl buffer at pH 7.5 was added to the cell paste of each sample. After thorough resuspension at room temperature on a plate mixer for 30 min, the cells were incubated at 40° C. for 1 hour in a PCR thermocycler. The samples were then centrifuged for 10 min at 4000 rpm and 4° C., and the clear supernatants were collected for ligation reactions.

The oligonucleotide mix for Reaction 2 in Example 4 were used as substrates in the ligation reaction. The lysate was screened in a 100 nanoliter reaction that comprised 50 mM Tris-HCl buffer at pH 7.5, 20 mM MgCl₂, 1 mM dithiothreitol (DTT), 8 mM adenosine triphosphate (ATP), and 1.25 mM of an equimolar mixture of the oligonucleotide fragments compromising Reaction 2. Nanoliter scale liquid transfers were carried out by Echo Acoustic Liquid Handler. The reactions were mixed by vortex, incubated for 2 hr at 37° C. in a PCR thermocycler, and quenched and diluted for analytical analysis by CE as described in Example 4. Using the peak area from CE analytical method, the % conversions of sense strand and antisense strand for each sample were calculated as shown below:

$$\% conversion = \frac{area under the curve for the product peak}{\begin{matrix}Total area under the curve for all \\ products and remaining fragments\end{matrix}} * 100$$

Activity relative to SEQ ID NO: 1416 (Activity FIOP) was calculated as the % conversion by the variant over the % conversion by SEQ ID NO: 1416. The result is shown in Table 13.1.

TABLE 13.1
Activities of Variants Relative to SEQ ID NO: 1416

| | Amino Acid | Activity FIOP on Sense | Activity FIOP on Antisense |
|---|---|---|---|
| SEQ ID | Differences | Strand | Strand |
| NO: | (Relative to | Relative to | Relative to |
| (nt/aa) | SEQ ID NO: 1416) | SEQ ID NO: 1416 | SEQ ID NO: 1416 |
| 1435/1436 | I83L/D137R/V150D | ++++ | |
| 1437/1438 | D137R/G213R | ++++ | +++ |
| 1439/1440 | D137R | ++++ | +++ |
| 1441/1442 | V150D/L190M | +++ | ++ |
| 1443/1444 | V143A/V150D | +++ | |
| 1445/1446 | L104T/V150D | +++ | ++ |
| 1447/1448 | P60A/V143A/V150D/L190M | +++ | ++ |
| 1449/1450 | P60A/V150D/L190M/G213R | +++ | + |
| 1451/1452 | L104T/D137R/C154V | +++ | ++ |
| 1453/1454 | D123K/D137R | ++ | ++++ |
| 1455/1456 | D137R/V150D/C154V/L190M | ++ | |
| 1457/1458 | D123K/V143T/V150D | ++ | ++++ |
| 1459/1460 | P60A/L104T/V143A/V150D/ | ++ | |
| | G213R | | |
| 1461/1462 | P60A/V95G/V143T/L190M | + | +++ |
| 1463/1464 | D123R/D137R/L190M | + | +++ |
| 1465/1466 | P60A/D123R | | ++++ |
| 1467/1468 | L104T/D123R | | ++++ |
| 1469/1470 | P60A/D123R/V143A | | ++++ |

Levels of increased activity on sense strand were determined relative to the reference polypeptide of SEQ ID NO: 1416 and defined as follows:
“+” > 1, “++” > 1.1, “+++” > 1.3, “++++” > 1.6
Levels of increased activity on antisense strand were determined relative to the reference polypeptide of SEQ ID NO: 1416 and defined as follows:
“+” > 1, “++” > 1.1, “+++” > 1.3, “++++” > 2

### Example 14

### Improvements Over SEQ ID NO: 1416 in RNA Ligase Activity

The RNA ligase of SEQ ID NO: 1416 was selected as the parent enzyme in an RNA ligation assay. Libraries of engineered genes were produced using known techniques (e.g., saturation mutagenesis, recombination of previously identified beneficial mutations). The polypeptides encoded by each gene were produced in high throughput (HTP) as described in Example 1, and the cell paste was generated as described in Example 2. To prepare lysate, 600 ul of 0.1 mg/mL lysozyme in 50 mM Tris-HCl buffer at pH 7.5 was added to the cell paste of each sample. After thorough resuspension at room temperature on a plate mixer for 1 hour, the cells were centrifuged for 20 min at 4000 rpm and 4° C. The supernatant was diluted 4-fold in 50 mM Tris-HCl buffer at pH 7.5 and incubated at 40° C. for 1 hour in a PCR thermocycler. The samples were then centrifuged for 10 min at 4000 rpm and 4° C., and the clear supernatants were collected for ligation reactions.

The oligonucleotide mix for Reaction 3 in Example 4 were used as substrates in the ligation reaction. The lysate was screened in a 100 nanoliter reaction that comprised 50 mM Tris-HCl buffer at pH 7.5, 20 mM MgCl₂, 1 mM dithiothreitol (DTT), 8 mM adenosine triphosphate (ATP), and 1.25 mM of an equimolar mixture of the oligonucleotide fragments compromising Reaction 3. Nanoliter scale liquid transfers were carried out by Echo Acoustic Liquid Handler. The reactions were mixed by vortex, incubated for 2 hr at 37° C. in a PCR thermocycler, and quenched and diluted for analytical analysis by CE as described in Example 4. Using the peak area from CE analytical method, the % conversions of sense strand and antisense strand for each sample were calculated as shown below:

$$\% conversion = \frac{area under the curve for the product peak}{\begin{matrix}Total area under the curve for all \\ products and remaining fragments\end{matrix}} * 100$$

Activity relative to SEQ ID NO: 1416 (Activity FIOP) was calculated as the % conversion by the variant over the % conversion by SEQ ID NO: 1416. The result is shown in Table 14.1.

TABLE 14.1: Activities of Variants Relative to SEQ ID NO: 1416

| | Amino Acid | Activity FIOP on | Activity FIOP on |
|---|---|---|---|
| SEQ ID | Differences | Antisense Strand | Sense Strand |
| NO: | (Relative to | relative to | relative to |
| (nt/aa) | SEQ ID NO: 1416) | SEQ ID NO: 1416 | SEQ ID NO: 1416 |
| 1477/1478 | M38V | ++++ | + |
| 1479/1480 | L249K | ++++ | |
| 1481/1482 | A255S | ++++ | |
| 1483/1484 | Y23K | ++++ | |
| 1485/1486 | G33L | ++++ | |
| 1487/1488 | A25G | ++++ | |
| 1489/1490 | I226R | ++++ | + |
| 1491/1492 | L249S | ++++ | |
| 1493/1494 | E29S | ++++ | |
| 1495/1496 | L37M | ++++ | + |
| 1497/1498 | M38A | ++++ | ++ |
| 1499/1500 | S10F | ++++ | |
| 1501/1502 | D250S | +++ | |
| 1503/1504 | G75D | +++ | ++ |
| 1505/1506 | T230V | +++ | |
| 1507/1508 | S77M | +++ | |
| 1509/1510 | S251E | +++ | ++ |
| 1511/1512 | Y23T | +++ | |
| 1513/1514 | G33V | +++ | |
| 1515/1516 | F228L | +++ | ++ |
| 1517/1518 | H81Y | +++ | ++ |
| 1519/1520 | Y224A | +++ | + |
| 1521/1522 | V64Q | +++ | + |
| 1523/1524 | S254K | +++ | + |
| 1525/1526 | E268F | ++ | |
| 1527/1528 | P65C | ++ | + |
| 1529/1530 | T232N | ++ | |
| 1531/1532 | L332E | ++ | + |
| 1533/1534 | L37K | ++ | |
| 1535/1536 | G331R | ++ | ++ |
| 1537/1538 | T330K | ++ | |
| 1539/1540 | A101P | ++ | |
| 1541/1542 | E268G | ++ | + |
| 1543/1544 | N307R | ++ | + |
| 1545/1546 | Y224Q | ++ | + |
| 1547/1548 | D137N | ++ | + |
| 1549/1550 | V223C | ++ | + |
| 1551/1552 | S280N | + | + |
| 1553/1554 | V289I | + | + |
| 1555/1556 | S288E | + | + |
| 1557/1558 | H342T | | +++ |
| 1559/1560 | V93K | | +++ |
| 1561/1562 | E268M | | ++ |
| 1563/1564 | R296Q | | ++ |
| 1565/1566 | K86G | | + |
| 1567/1568 | S241Q | | ++++ |
| 1569/1570 | D126M | | + |
| 1571/1572 | N277A | | ++ |
| 1573/1574 | K87E | | ++ |
| 1575/1576 | A247I | +++ | |
| 1577/1578 | F243H | ++ | |
| 1579/1580 | I84V | ++ | |
| 1581/1582 | I83A | ++ | |
| 1583/1584 | D192E | + | |
| 1585/1586 | V105K | + | |
| 1587/1588 | A25G/E29S | +++ | ++++ |
| 1589/1590 | A25G/E29S/D250S | ++ | +++ |
| 1591/1592 | A25G/E29S/G75D/S77M/D250S/A255S | +++ | ++ |
| 1593/1594 | A25G/G75D/T230V | ++ | ++ |
| 1595/1596 | A25G/S77M/I226R/A255S | +++ | +++ |
| 1597/1598 | A25G/T230V | ++ | ++++ |
| 1599/1600 | D250S/A255S | ++ | + |
| 1601/1602 | E29S/G75D/I226R/T230V/A255S | +++ | |
| 1603/1604 | E29S/G75D/S77M | +++ | + |
| 1605/1606 | E29S/G75D/S77M/I226R | + | + |
| 1607/1608 | E29S/G75D/S77M/T230V/A255S | + | +++ |
| 1609/1610 | E29S/I226R | +++ | ++++ |
| 1611/1612 | E29S/I226R/T230V | ++++ | +++ |
| 1613/1614 | E29S/T230V/A255S | ++ | |
| 1615/1616 | G33V/F228L | +++ | +++ |
| 1617/1618 | G33V/L249K | ++ | |
| 1619/1620 | G33V/L249S | +++ | |
| 1621/1622 | G33V/L37M/F228L | ++ | |
| 1623/1624 | G33V/L37M/M38A/F228L | ++++ | |
| 1625/1626 | G33V/L37M/M38A/F228L/L249K | +++ | |
| 1627/1628 | G33V/M38V | +++ | |
| 1629/1630 | G75D/D250S/A255S | + | |
| 1631/1632 | G75D/I226R | ++ | ++++ |
| 1633/1634 | G75D/I226R/D250S/A255S | ++ | |
| 1635/1636 | G75D/I226R/T230V/D250S/A255S | ++ | +++ |
| 1637/1638 | G75D/S77M | +++ | + |
| 1639/1640 | G75D/S77M/A255S | ++ | |
| 1641/1642 | G75D/S77M/D250S/A255S | +++ | +++ |
| 1643/1644 | G75D/S77M/I226R/A255S | + | ++++ |
| 1645/1646 | G75D/S77M/I226R/D250S/A255S | + | |
| 1647/1648 | G75D/S77M/T230V | ++++ | +++ |
| 1649/1650 | G75D/S77M/T230V/D250S | +++ | ++++ |
| 1651/1652 | G75D/T230V | +++ | ++++ |
| 1653/1654 | G75D/T230V/A255S | +++ | |
| 1655/1656 | G75D/T230V/D250S | ++++ | ++++ |
| 1657/1658 | I226R/A255S | + | ++++ |
| 1659/1660 | I226R/D250S | ++ | ++++ |
| 1661/1662 | I226R/T230V | +++ | ++++ |
| 1663/1664 | I226R/T230V/A255S | ++ | +++ |
| 1665/1666 | L37M/F228L | + | +++ |
| 1667/1668 | L37M/F228L/L249S | ++ | |
| 1669/1670 | L37M/M38A | + | +++ |
| 1671/1672 | L37M/M38A/F228L | +++ | |
| 1673/1674 | L37M/M38V/F228L/L249K | + | |
| 1675/1676 | L37M/M38V/L249K | + | + |
| 1677/1678 | M38A/L249S/A291T | + | ++++ |
| 1679/1680 | S10F/F228L | + | |
| 1681/1682 | S10F/F228L/L249K | ++ | |
| 1683/1684 | S10F/G33L/L249K | ++ | |
| 1685/1686 | S10F/G33L/L37M | ++ | + |
| 1687/1688 | S10F/G33L/L37M/M38A/L249K | ++ | +++ |
| 1689/1690 | S10F/G33L/M38A/L249S | +++ | |
| 1691/1692 | S10F/G33L/M38V | ++ | +++ |
| 1693/1694 | S10F/G33V/L249K/S251E | ++ | +++ |
| 1695/1696 | S10F/G33V/L37M | +++ | +++ |
| 1697/1698 | S10F/G33V/L37M/F228L | +++ | |
| 1699/1700 | S10F/G33V/L37M/L249K | ++ | |
| 1701/1702 | S10F/G33V/L37M/L249S | + | |
| 1703/1704 | S10F/G33V/L37M/M38A/F228L/L249K | +++ | |
| 1705/1706 | S10F/G33V/L37M/M38V | ++ | |
| 1707/1708 | S10F/G33V/L37M/M38V/F228L/L249K | +++ | |
| 1709/1710 | S10F/G33V/L37M/M38V/L249K | ++ | +++ |
| 1711/1712 | S10F/L249K | ++ | +++ |
| 1713/1714 | S10F/L249S | + | |
| 1715/1716 | S10F/L37M | ++ | +++ |
| 1717/1718 | S10F/L37M/F228L | + | ++++ |
| 1719/1720 | S10F/L37M/F228L/L249K | ++ | +++ |
| 1721/1722 | S10F/L37M/L249K | +++ | +++ |
| 1723/1724 | S10F/L37M/M38A/F228L | ++ | ++ |
| 1725/1726 | S10F/L37M/M38A/F228L/L249K | ++ | +++ |
| 1727/1728 | S10F/L37M/M38V | ++ | + |
| 1729/1730 | S10F/L37M/M38V/F228L | +++ | |
| 1731/1732 | S10F/L37M/M38V/F228L/L249S | +++ | +++ |
| 1733/1734 | S10F/M38A/L249K | +++ | + |
| 1735/1736 | S10F/M38V | +++ | +++ |
| 1737/1738 | S10F/M38V/F228L | ++ | +++ |
| 1739/1740 | S10F/M38V/L249S | +++ | + |
| 1741/1742 | S77M/A255S | +++ | |
| 1743/1744 | S77M/D250S | ++ | |
| 1745/1746 | S77M/D250S/A255S | +++ | |
| 1747/1748 | S77M/I226R | +++ | + |
| 1749/1750 | S77M/I226R/A255S | +++ | ++++ |
| 1751/1752 | S77M/T230V | +++ | +++ |
| 1753/1754 | S77M/T230V/A255S | +++ | ++ |
| 1755/1756 | T230V/A255S | +++ | ++++ |
| 1757/1758 | T230V/D250S | +++ | ++++ |
| 1759/1760 | T230V/D250S/A255S | + | |
| 1761/1762 | Y23T/A25G/I226R/A255S | + | ++++ |
| 1763/1764 | Y23T/A25G/I226R/T230V | ++++ | +++ |
| 1765/1766 | Y23T/A25G/T230V | +++ | +++ |

Levels of increased activity were determined for Activity FIOP on Antisense Strand relative to SEQ ID NO: 1416 and are defined as follows:
“+” > 1.0, “++” > 1.1, “+++” > 1.3, “++++” > 1.7. Levels of
increased activity were determined for Activity FIOP on Sense Strand relative to SEQ ID NO: 1416 and are defined as follows:
“+” > 1.0, “++” > 1.2, “+++” > 1.5, “++++” > 3.0.

### Example 15

### Improvements Over SEQ ID NO: 1612 in RNA Ligase Activity

The RNA ligase of SEQ ID NO: 1612 was selected as the parent enzyme in an RNA ligation assay. Libraries of engineered genes were produced using known techniques (e.g., saturation mutagenesis, recombination of previously identified beneficial mutations). The polypeptides encoded by each gene were produced in high throughput (HTP) as described in Example 1, and the cell paste was generated as described in Example 2. To prepare lysate, 600 ul of 0.1 mg/mL lysozyme in 50 mM Tris-HCl buffer at pH 7.5 was added to the cell paste of each sample. After thorough resuspension at room temperature on a plate mixer for 1 hour, the cells were centrifuged for 20 min at 4000 rpm and 4° C. The supernatant was diluted 4-fold in 50 mM Tris-HCl buffer at pH 7.5 and incubated at 40° C. for 1 hour in a PCR thermocycler. The samples were then centrifuged for 10 min at 4000 rpm and 4° C., and the clear supernatants were collected for ligation reactions.

The oligonucleotide mix for Reaction 3 in Example 4 were used as substrates in the ligation reaction. The lysate was screened in a 100 nanoliter reaction that comprised 50 mM Tris-HCl buffer at pH 7.5, 20 mM MgCl₂, 1 mM dithiothreitol (DTT), 8 mM adenosine triphosphate (ATP), and 1.25 mM of an equimolar mixture of the oligonucleotide fragments compromising Reaction 3. Nanoliter scale liquid transfers were carried out by Echo Acoustic Liquid Handler. The reactions were mixed by vortex, incubated for 2 hr at 37° C. in a PCR thermocycler, and quenched and diluted for analytical analysis by CE as described in Example 4. Using the peak area from CE analytical method, the % conversions of sense strand and antisense strand for each sample were calculated as shown below:

$$\% conversion = \frac{area under the curve for the product peak}{\begin{matrix}Total area under the curve for all \\ products and remaining fragments\end{matrix}} * 100$$

Activity relative to SEQ ID NO: 1612 (Activity FIOP) was calculated as the % conversion by the variant over the % conversion by SEQ ID NO: 1612. The result is shown in Table 15.1.

TABLE 15.1: Activities of Variants Relative to SEQ ID NO: 1612

| | Amino Acid | Activity FIOP on | Activity FIOP on |
|---|---|---|---|
| SEQ ID | Differences | Antisense Strand | Sense Strand |
| NO: | (Relative to | relative to | relative to |
| (nt/aa) | SEQ ID NO: 1612) | SEQ ID NO: 1612 | SEQ ID NO: 1612 |
| 1767/1768 | A101P | + | ++ |
| 1769/1770 | A291T | +++ | ++ |
| 1771/1772 | A291T/N307R | | ++++ |
| 1773/1774 | G331R | +++ | |
| 1775/1776 | G33V/G75D | ++ | ++ |
| 1777/1778 | G33V/G75D/L249K/A291T/G331R | ++ | |
| 1779/1780 | G33V/M38A | + | |
| 1781/1782 | G33V/V93K/L249K | | + |
| 1783/1784 | M38A/G75D/A291T/N307R | ++ | |
| 1785/1786 | M38V/G75D/L249K/D250S | + | + |
| 1787/1788 | M38V/G75D/V93K/S271G | ++ | + |
| 1789/1790 | M38V/V93K | + | |
| 1791/1792 | S10F/G75D | ++ | |
| 1793/1794 | S10F/M38A/G75D | ++ | ++++ |
| 1795/1796 | S10F/M38V/G75D/A291T | ++ | ++ |
| 1797/1798 | S10F/M38V/G75D/L249K | ++ | +++ |
| 1799/1800 | S10F/M38V/G75D/V93K | ++ | |
| 1801/1802 | V64Q/Y224A | + | +++ |
| 1803/1804 | Y224A | + | + |
| 1805/1806 | Y224Q | ++ | ++++ |
| 1807/1808 | Y23T/A101P/N307K | +++ | ++ |
| 1809/1810 | Y23T/E268F | ++ | ++ |
| 1811/1812 | Y23T/L37K/V64Q/Y224A/S254K | ++++ | ++++ |
| 1813/1814 | Y23T/V64Q/H81Y | +++ | |
| 1815/1816 | Y23T/Y224A/E268F | ++ | ++ |
| 1817/1818 | Y23T/Y224A/S254K | +++ | +++ |
| 1819/1820 | Y23T | ++++ | +++ |
| 1821/1822 | Y23T/V64Q/Y224A | +++ | ++++ |
| 1823/1824 | V64Q | +++ | +++ |
| 1825/1826 | G75D/L249K/N307R | +++ | + |
| 1827/1828 | V93K | ++++ | + |
| 1829/1830 | G33V/M38A/G75D/L249K | ++++ | ++++ |
| 1831/1832 | G75D/L249K | +++ | |
| 1833/1834 | A25S/M38V/G75D/V93K/A291T | +++ | +++ |
| 1835/1836 | G33V/M38A/G75D | ++++ | +++ |
| 1837/1838 | G75D | ++++ | ++++ |
| 1839/1840 | Y23T/S254K | ++++ | +++ |

Levels of increased activity were determined for Activity FIOP on Antisense Strand relative to SEQ ID NO: 1612 and are defined as follows:
“+” > 1.0, “++” > 1.2, “+++” > 1.7, “++++” > 3.0. Levels of increased
activity were determined for Activity FIOP on Sense Strand relative to SEQ ID NO: 1612 and are defined as follows:
“+” > 1.0, “++” > 1.2, “+++” > 1.5, “++++” > 2.5.

### Example 16

### Improvements Over SEQ ID NO: 1840 in RNA Ligase Activity

The RNA ligase of SEQ ID NO: 1840 was selected as the parent enzyme in an RNA ligation assay. Libraries of engineered genes were produced using known techniques (e.g., saturation mutagenesis, recombination of previously identified beneficial mutations). The polypeptides encoded by each gene were produced in high throughput (HTP) as described in Example 1, and the cell paste was generated as described in Example 2. To prepare lysate, 600 ul of 0.1 mg/mL lysozyme in 50 mM Tris-HCl buffer at pH 7.5 was added to the cell paste of each sample. After thorough resuspension at room temperature on a plate mixer for 1 hour, the cells were centrifuged for 20 min at 4000 rpm and 4° C. The supernatant was incubated at 40° C. for 1 hour in a PCR thermocycler. The samples were then centrifuged for 10 min at 4000 rpm and 4° C., and the clear supernatants were collected for ligation reactions.

The oligonucleotide mix for Reaction 3 in Example 4 were used as substrates in the ligation reaction. The lysate was screened in a 100 nanoliter reaction that comprised 50 mM Tris-HCl buffer at pH 7.5, 20 mM MgCl₂, 1 mM dithiothreitol (DTT), 8 mM adenosine triphosphate (ATP), and 1.75 mM of an equimolar mixture of the oligonucleotide fragments compromising Reaction 3. Nanoliter scale liquid transfers were carried out by Echo Acoustic Liquid Handler. The reactions were mixed by vortex, incubated for 2 hr at 37° C. in a PCR thermocycler, and quenched and diluted for analytical analysis by CE as described in Example 4. Using the peak area from CE analytical method, the % conversions of sense strand and antisense strand for each sample were calculated as shown below:

$$\% conversion = \frac{area under the curve for the product peak}{\begin{matrix}Total area under the curve for all \\ products and remaining fragments\end{matrix}} * 100$$

Activity relative to SEQ ID NO: 1840 (Activity FIOP) was calculated as the % conversion by the variant over the % conversion by SEQ ID NO: 1840. The result is shown in Table 16.1.

TABLE 16.1
Activities of Variants Relative to SEQ ID NO: 1840

| | Amino Acid | Activity FIOP on | Activity FIOP on |
|---|---|---|---|
| SEQ | Differences | Antisense Strand | Sense Strand |
| ID NO: | (Relative to SEQ | relative to SEQ | relative to SEQ |
| (nt/aa) | ID NO: 1840) | ID NO: 1840 | ID NO: 1840 |
| 1841/1842 | M38A/P65C/D192E | ++++ | + |
| 1843/1844 | M38T/P65C | ++++ | ++ |
| 1845/1846 | M38A/P65C/Y224R | ++++ | + |
| 1847/1848 | M38A/V64Q | ++++ | ++++ |
| 1849/1850 | V93K/N307R | ++++ | +++ |
| 1851/1852 | M38A | ++++ | +++ |
| 1853/1854 | M38A/Y224A | +++ | +++ |
| 1855/1856 | M38A/D192E/Y224R | +++ | +++ |
| 1857/1858 | P65C | +++ | |
| 1859/1860 | M38A/D192E/Y224A | +++ | +++ |
| 1861/1862 | D192E/Y224A | +++ | +++ |
| 1863/1864 | G75D/N307R | +++ | + |
| 1865/1866 | G75D/D250I/E268G/ | +++ | ++ |
| | N307R | | |
| 1867/1868 | D250I/E268G/N307R/ | +++ | ++ |
| | H342T | | |
| 1869/1870 | Y224R | ++ | +++ |
| 1871/1872 | P65C/D192E | ++ | ++ |
| 1873/1874 | E268G | ++ | +++ |
| 1875/1876 | V64Q/D192E | ++ | ++ |
| 1877/1878 | D250I/E268G/A291T/ | ++ | ++ |
| | H342T | | |
| 1879/1880 | G75D/V93K/L249K/ | ++ | |
| | N307R/H342T | | |
| 1881/1882 | D250I/A291T | ++ | +++ |
| 1883/1884 | M38A/V64Q/D192E | ++ | ++++ |
| 1885/1886 | M38A/V64Q/Y224R | ++ | ++ |
| 1887/1888 | V93K/L249K/N307R | ++ | + |
| 1889/1890 | M38A/V64Q/K86G/ | ++ | ++ |
| | D192E/S241R | | |
| 1891/1892 | M38A/H342T | ++ | + |
| 1893/1894 | D192E/Y224R | + | ++ |
| 1895/1896 | K248G/D250I/N307R | + | ++++ |
| 1897/1898 | G75D/S77M/D250I/ | + | + |
| | E268G/N307R/H342T | | |
| 1899/1900 | V93K/E268G | + | ++ |
| 1901/1902 | D192E | + | ++ |
| 1903/1904 | M38A/D192E | + | ++++ |
| 1905/1906 | V64Q | + | ++++ |

Levels of increased activity were determined for Activity FIOP on Antisense Strand relative to SEQ ID NO: 1840 and are defined as follows: “+” > 1.2, “++” > 1.23, “+++” > 1.3, “++++” > 1.4. Levels of increased activity were determined for Activity FIOP on Sense Strand relative to SEQ ID NO: 1840 and are defined as follows: “+” > 1.0, “++” > 1.1, “+++” > 1.3, “++++” > 1.55.

### Example 17

### Improvements Over SEQ ID NO: 1416 in RNA Ligase Activity

The RNA ligase of SEQ ID NO: 1416 was selected as the parent enzyme in an RNA ligation assay. Libraries of engineered genes were produced using known techniques (e.g., saturation mutagenesis, recombination of previously identified beneficial mutations). The polypeptides encoded by each gene were produced in high throughput (HTP) as described in Example 1, and the cell paste was generated as described in Example 2. To prepare lysate, 600 ul of 0.1 mg/mL lysozyme in 50 mM Tris-HCl buffer at pH 7.5 was added to the cell paste of each sample. After thorough resuspension at room temperature on a plate mixer for 1 hour, the cells were centrifuged for 20 min at 4000 rpm and 4° C. The supernatant was incubated at 40° C. for 1 hour in a PCR thermocycler. The samples were then centrifuged for 10 min at 4000 rpm and 4° C., and the clear supernatants were collected for ligation reactions.

The oligonucleotide mix for Reaction 4 in Example 4 were used as substrates in the ligation reaction. The lysate was screened in a 100 nanoliter reaction that comprised 50 mM Tris-HCl buffer at pH 7.5, 20 mM MgCl₂, 1 mM dithiothreitol (DTT), 8 mM adenosine triphosphate (ATP), and 1.25 mM of an equimolar mixture of the oligonucleotide fragments compromising Reaction 4. Nanoliter scale liquid transfers were carried out by Echo Acoustic Liquid Handler. The reactions were mixed by vortex, incubated for 2 hr at 37° C. in a PCR thermocycler, and quenched and diluted for analytical analysis by CE as described in Example 4. Using the peak area from CE analytical method, the % conversions of sense strand and antisense strand for each sample were calculated as shown below:

$$\% conversion = \frac{area under the curve for the product peak}{\begin{matrix}Total area under the curve for all \\ products and remaining fragments\end{matrix}} * 100$$

Activity relative to SEQ ID NO: 1416 (Activity FIOP) was calculated as the % conversion by the variant over the % conversion by SEQ ID NO: 1416. The result is shown in Table 17.1.

TABLE 17.1
Activities of Variants Relative to SEQ ID NO: 1416

| | Amino Acid | Activity FIOP on | Activity FIOP on |
|---|---|---|---|
| SEQ | Differences | Antisense Strand | Sense Strand |
| ID NO: | (Relative to SEQ | relative to SEQ | relative to SEQ |
| (nt/aa) | ID NO: 1416) | ID NO: 1416 | ID NO: 1416 |
| 1481/1482 | A255S | +++ | +++ |
| 1507/1508 | S77M | ++ | +++ |
| 1509/1510 | S251E | ++ | ++ |
| 1511/1512 | Y23T | ++ | + |
| 1527/1528 | P65C | ++++ | ++++ |
| 1529/1530 | T232N | + | |
| 1539/1540 | A101P | ++++ | ++++ |
| 1551/1552 | S280N | +++ | ++++ |
| 1563/1564 | R296Q | + | ++ |
| 1571/1572 | N277A | ++ | ++ |
| 1581/1582 | I83A | + | + |
| 1907/1908 | K86D | ++++ | ++++ |
| 1909/1910 | A255V | ++++ | ++++ |
| 1911/1912 | I306T | ++++ | ++++ |
| 1913/1914 | D250I | +++ | +++ |
| 1915/1916 | G75A | +++ | +++ |
| 1917/1918 | F318S | +++ | +++ |
| 1919/1920 | N335Q | +++ | +++ |
| 1921/1922 | S241G | +++ | ++ |
| 1923/1924 | D250E | ++ | ++ |
| 1925/1926 | K248F | ++ | |
| 1927/1928 | V344K | ++ | ++ |
| 1929/1930 | M38S | ++ | ++ |
| 1931/1932 | E272S | ++ | + |
| 1933/1934 | I83G | ++ | ++ |
| 1935/1936 | I306L | ++ | ++ |
| 1937/1938 | E272M | + | + |
| 1939/1940 | K248G | + | + |
| 1941/1942 | V257E | + | + |
| 1943/1944 | E258M | + | + |
| 1945/1946 | S323G | + | + |

Levels of increased activity were determined for Activity FIOP on Antisense Strand relative to SEQ ID NO: 1416 and are defined as follows: “+” > 1.1, “++” > 1.12, “+++” > 1.17, “++++” > 1.3. Levels of increased activity were determined for Activity FIOP on Sense Strand relative to SEQ ID NO: 1416 and are defined as follows: “+” > 1.0, “++” > 1.1, “+++” > 1.2, “++++” > 1.35.

### Example 18

### Improvements Over SEQ ID NO: 1866 in RNA Ligase Activity

The RNA ligase of SEQ ID NO: 1866 was selected as the parent enzyme in an RNA ligation assay. Libraries of engineered genes were produced using known techniques (e.g., saturation mutagenesis, recombination of previously identified beneficial mutations). The polypeptides encoded by each gene were produced in high throughput (HTP) as described in Example 1, and the cell paste was generated as described in Example 2. To prepare lysate, 600 ul of 0.1 mg/mL lysozyme in 50 mM Tris-HCl buffer at pH 7.5 was added to the cell paste of each sample. After thorough resuspension at room temperature on a plate mixer for 1 hour, the cells were centrifuged for 20 min at 4000 rpm and 4° C. The supernatant was incubated at 40° C. for 1 hour in a PCR thermocycler. The samples were then centrifuged for 10 min at 4000 rpm and 4° C., and the clear supernatants were collected for ligation reactions.

The oligonucleotide mix for Reaction 4 in Example 4 were used as substrates in the ligation reaction. The lysate was screened in a 100 nanoliter reaction that comprised 50 mM Tris-HCl buffer at pH 7.5, 20 mM MgCl₂, 1 mM dithiothreitol (DTT), 8 mM adenosine triphosphate (ATP), and 1.25 mM of an equimolar mixture of the oligonucleotide fragments compromising Reaction 4. Nanoliter scale liquid transfers were carried out by Echo Acoustic Liquid Handler. The reactions were mixed by vortex, incubated for 2 hr at 37° C. in a PCR thermocycler, and quenched and diluted for analytical analysis by CE as described in Example 4. Using the peak area from CE analytical method, the % conversions of sense strand and antisense strand for each sample were calculated as shown below:

$$\% conversion = \frac{area under the curve for the product peak}{\begin{matrix}Total area under the curve for all \\ products and remaining fragments\end{matrix}} * 100$$

Activity relative to SEQ ID NO: 1866 (Activity FIOP) was calculated as the % conversion by the variant over the % conversion by SEQ ID NO: 1866. The result is shown in Table 18.1.

TABLE 18.1
Activities of Variants Relative to SEQ ID NO: 1866

| | Amino Acid | Activity FIOP on | Activity FIOP on |
|---|---|---|---|
| SEQ | Differences | Antisense Strand | Sense Strand |
| ID NO: | (Relative to SEQ | relative to SEQ | relative to SEQ |
| (nt/aa) | ID NO: 1866) | ID NO: 1866 | ID NO: 1866 |
| 1947/1948 | M38S/K248F/S251E | ++++ | ++++ |
| 1949/1950 | M38S/K248F/I250E/S251E/N277A | ++++ | ++++ |
| 1951/1952 | K248F/I250E/S251E | ++++ | ++++ |
| 1953/1954 | M38S/V93K/D192E/K248F/S251E | ++++ | ++++ |
| 1955/1956 | M38S/D192E/K248F/S251E | ++++ | +++ |
| 1957/1958 | I250E/S251E | ++++ | ++++ |
| 1959/1960 | M38S/K248F/I250E/S251E | +++ | ++++ |
| 1961/1962 | K248F/S251E/V344K | +++ | +++ |
| 1963/1964 | D192E/K248F/I250E/S251R | +++ | +++ |
| 1965/1966 | M38S/K248F/I250E | +++ | +++ |
| 1967/1968 | M38S/V93K/I250E/S251E/N277A | +++ | ++++ |
| 1969/1970 | M38A/A255V/S280N/F318S | +++ | +++ |
| 1971/1972 | K248F | +++ | ++ |
| 1973/1974 | M38A/K86D/S241G/A255V/ | +++ | +++ |
| | S280N/F318S/N335Q | | |
| 1975/1976 | M38A/A255S | +++ | +++ |
| 1977/1978 | M38A | +++ | +++ |
| 1979/1980 | F318S | ++ | +++ |
| 1981/1982 | M38A/A101P/A255S/S280N | ++ | +++ |
| 1983/1984 | M38A/F318S/N335Q | ++ | ++ |
| 1985/1986 | M38A/A255V/F318S | ++ | ++ |
| 1987/1988 | D192E/I250E/S251E/V344K | ++ | +++ |
| 1989/1990 | M38A/N335Q | ++ | ++ |
| 1991/1992 | F318S/N335Q | ++ | +++ |
| 1993/1994 | M38S/N277A | ++ | +++ |
| 1995/1996 | M38S/V93K/S251E | ++ | +++ |
| 1997/1998 | D192E/S251E | ++ | ++ |
| 1999/2000 | A255S | ++ | ++ |
| 2001/2002 | M38A/V64Q/F318S | ++ | ++ |
| 2003/2004 | K248I | ++ | + |
| 2005/2006 | M38A/V64Q/S241G/A255V/F318S | ++ | ++ |
| 2007/2008 | S251E | + | ++ |
| 2009/2010 | V64Q/A101P/A255V/S280N/F318S | + | + |
| 2011/2012 | M38A/A101P/A255V/S280N/F318S/N335Q | + | + |
| 2013/2014 | M38A/A255V/S280N | + | + |
| 2015/2016 | M38S/S251R/N277A | + | ++ |
| 2017/2018 | V64Q/F318S | + | + |
| 2019/2020 | M38A/V64Q/A255V/F318S | + | + |
| 2021/2022 | S241G/A255S/S280N/F318S | + | + |
| 2023/2024 | A255V | + | + |
| 2025/2026 | M38A/K86D/A101P/S241G/F318S | + | + |
| 2027/2028 | M38A/S241G | + | ++ |
| 2029/2030 | M38A/A255S/F318S/N335Q | + | + |
| 2031/2032 | M38A/V64Q/A101P/F318S | + | + |
| 2033/2034 | M38A/V64Q/A255V/S280N/F318S | + | + |
| 2035/2036 | S241G/A255S/F318S | + | + |
| 2037/2038 | M38A/S241G/N335Q | + | |

Levels of increased activity were determined for Activity FIOP on Antisense Strand relative to SEQ ID NO: 1866 and are defined as follows: “+” > 1.0, “++” > 1.2, “+++” > 1.4, “++++” > 1.9. Levels of increased activity were determined for Activity FIOP on Sense Strand relative to SEQ ID NO: 1866 and are defined as follows: “+” > 1.0, “++” > 1.5, “+++” > 2.0, “++++” > 3.0.

### Example 19

### Improvements Over SEQ ID NO: 1954 in RNA Ligase Activity

SEQ ID NO: 1954 was selected as the parent enzyme in an RNA ligation assay. Libraries of engineered genes were produced using known techniques (e.g., saturation mutagenesis, recombination of previously identified beneficial mutations). The polypeptides encoded by each gene were produced in high throughput (HTP) as described in Example 1, and the cell paste was generated as described in Example 2. To prepare lysate, 600 ul of 0.1 mg/mL lysozyme in 50 mM Tris-HCl buffer at pH 7.5 was added to the cell paste of each sample. After thorough resuspension at room temperature on a plate mixer for 1 hour, the cells were centrifuged for 20 min at 4000 rpm and 4° C. The supernatant was diluted 2-fold in 50 mM Tris-HCl buffer at pH 7.5 and incubated at 40° C. for 1 hour in a PCR thermocycler. The samples were then centrifuged for 10 min at 4000 rpm and 4° C., and the clear supernatants were collected for ligation reactions.

The oligonucleotide mix for Reaction 5 in Example 4 were used as substrates in the ligation reaction. The lysate was screened in a 100 nanoliter reaction that comprised 50 mM Tris-HCl buffer at pH 7.5, 20 mM MgCl₂, 1 mM dithiothreitol (DTT), 8 mM adenosine triphosphate (ATP), and 1.25 mM of an equimolar mixture of the oligonucleotide fragments compromising Reaction 5. Nanoliter scale liquid transfers were carried out by Echo Acoustic Liquid Handler. The reactions were mixed by vortex, incubated for 2 hr at 33° C. in a PCR thermocycler, and quenched and diluted for analytical analysis by CE as described in Example 4. Using the peak area from CE analytical method, the % conversions of sense strand and antisense strand for each sample were calculated as shown below:

$$\% conversion = \frac{area under the curve for the product peak}{\begin{matrix}Total area under the curve for all \\ products and remaining fragments\end{matrix}} * 100$$

Activity relative to SEQ ID NO: 1954 (Activity FIOP) was calculated as the % conversion by the variant over the % conversion by SEQ ID NO: 1954. The result is shown in Table 19.1.

TABLE 19.1
RNA ligase 2 activity relative to SEQ ID NO: 1954

| | Amino Acid | Activity FIOP on | Activity FIOP on |
|---|---|---|---|
| SEQ | Differences | Antisense Strand | Sense Strand |
| ID NO: | (Relative to SEQ | relative to SEQ | relative to SEQ |
| (nt/aa) | ID NO: 1954) | ID NO: 1954 | ID NO: 1954 |
| 2039/2040 | K93V/T232N/A291T/I306L | ++++ | ++++ |
| 2041/2042 | K93V/T232N/S280N/I306L | ++++ | ++++ |
| 2043/2044 | K93V/S241G/V257E/I306L/N335Q | ++++ | ++++ |
| 2045/2046 | T232N/S280N | ++++ | ++++ |
| 2047/2048 | K93V/A291T/I306L | ++++ | +++ |
| 2049/2050 | K93V/V257E | ++++ | ++++ |
| 2051/2052 | K93V/I306L | ++++ | +++ |
| 2053/2054 | K93V/S280N | ++++ | +++ |
| 2055/2056 | E192D/F318S | ++++ | ++ |
| 2057/2058 | K93V/S280N/A291T | ++++ | +++ |
| 2059/2060 | K93V/S241G/A291T/I306L | +++ | ++ |
| 2061/2062 | E192D | +++ | ++ |
| 2063/2064 | V257E/A291T | +++ | +++ |
| 2065/2066 | S241G/V257E/I306L | +++ | +++ |
| 2067/2068 | S280N/N335Q | +++ | ++ |
| 2069/2070 | K93V/A291T/I306L/N335Q | +++ | ++ |
| 2071/2072 | K93V/N335Q | +++ | ++ |
| 2073/2074 | K93V/T232N/N335Q | ++ | ++ |
| 2075/2076 | V64Q/F318S | ++ | ++ |
| 2077/2078 | K93V/S241G | ++ | ++ |
| 2079/2080 | K93V/T232N/S241G/S280N | ++ | ++ |
| 2081/2082 | V64Q/E192D/F318S/S323G | ++ | + |
| 2083/2084 | T232N/S280N/I306L | ++ | ++ |
| 2085/2086 | K93V/T232N | ++ | ++ |
| 2087/2088 | A291T/N335Q | ++ | ++ |
| 2089/2090 | V257E/E258M | ++ | +++ |
| 2091/2092 | K93V/T232N/S241G/V257E/E258M/S280N/N335Q | ++ | +++ |
| 2093/2094 | F318S/S323G | ++ | + |
| 2095/2096 | K93V/S241G/A291T/N335Q | ++ | ++ |
| 2097/2098 | I83A/K86D/S323G | ++ | + |
| 2099/2100 | V257E/E258M/A291T | + | ++ |
| 2101/2102 | A101P/Y224A/F318S | + | + |
| 2103/2104 | I83A/E192D/A255V/R296Q | + | ++ |
| 2105/2106 | A171S/T232N/V257E/E258M/S280N | + | ++ |
| 2107/2108 | I83A/Y224A/A255V/F318S | + | + |
| 2109/2110 | V64Q/E192D/I250E/F318S | + | + |
| 2111/2112 | A101P/R296Q | + | ++ |
| 2113/2114 | T232N/S241G/S280N | + | + |
| 2115/2116 | V64Q/A255V/F318S | + | + |
| 2117/2118 | K86D/I250E/A255V | + | + |
| 2119/2120 | I83A/K86D/A101P | + | + |
| 2121/2122 | I83A/K86D | + | + |
| 2123/2124 | K93V/S241G/E258M/N335Q | + | + |
| 2125/2126 | I250E/R296Q/S323G | + | + |
| 2127/2128 | V257E/E258M/S280N | + | ++ |

Levels of increased activity were determined for Activity FIOP on Antisense Strand relative to SEQ ID NO: 1954 and are defined as follows: “+” > 1.0, “++” > 1.2, “+++” > 1.4, “++++” > 1.5. Levels of increased activity were determined for Activity FIOP on Sense Strand relative to SEQ ID NO: 1954 and are defined as follows: “+” > 1.0, “++” > 1.2, “+++” > 1.4, “++++” > 1.55.

### Example 20

### Improvements Over SEQ ID NO: 1866 in RNA Ligase Activity

SEQ ID NO: 1866 was selected as the parent enzyme in an RNA ligation assay. Libraries of engineered genes were produced using known techniques (e.g., saturation mutagenesis, recombination of previously identified beneficial mutations). The polypeptides encoded by each gene were produced in high throughput (HTP) as described in Example 1, and the cell paste was generated as described in Example 2. To prepare lysate, 600 ul of 0.1 mg/mL lysozyme in 50 mM Tris-HCl buffer at pH 7.5 was added to the cell paste of each sample. After thorough resuspension at room temperature on a plate mixer for 1 hour, the cells were centrifuged for 20 min at 4000 rpm and 4° C. The supernatant was incubated at 40° C. for 1 hour in a PCR thermocycler. The samples were then centrifuged for 10 min at 4000 rpm and 4° C., and the clear supernatants were collected for ligation reactions.

The oligonucleotide mix for Reaction 6 in Example 4 were used as substrates in the ligation reaction. The lysate was screened in a 100 nanoliter reaction that comprised 50 mM Tris-HCl buffer at pH 7.5, 20 mM MgCl₂, 1 mM dithiothreitol (DTT), 8 mM adenosine triphosphate (ATP), and 1.25 mM of an equimolar mixture of the oligonucleotide fragments compromising Reaction 6. Nanoliter scale liquid transfers were carried out by Echo Acoustic Liquid Handler. The reactions were mixed by vortex, incubated for 2 hr at 33° C. in a PCR thermocycler, and quenched and diluted for analytical analysis by CE as described in Example 4. Using the peak area from CE analytical method, the % conversions of sense strand and antisense strand for each sample were calculated as shown below:

$$\% conversion = \frac{area under the curve for the product peak}{\begin{matrix}Total area under the curve for all \\ products and remaining fragments\end{matrix}} * 100$$

Activity relative to SEQ ID NO: 1866 (Activity FIOP) was calculated as the % conversion by the variant over the % conversion by SEQ ID NO: 1866. The result is shown in Table 20.1.

TABLE 20.1
RNA ligase 2 activity relative to SEQ ID NO: 1866

| | Amino Acid | Activity FIOP on | Activity FIOP on |
|---|---|---|---|
| SEQ | Differences | Antisense Strand | Sense Strand |
| ID NO: | (Relative to SEQ | relative to SEQ | relative to SEQ |
| (nt/aa) | ID NO: 1866) | ID NO: 1866 | ID NO: 1866 |
| 2129/2130 | M89D | ++++ | ++++ |
| 2131/2132 | M89E | ++++ | ++++ |
| 2133/2134 | V122F | ++++ | +++ |
| 2135/2136 | K181P | ++++ | ++++ |
| 2137/2138 | I28V | ++++ | ++++ |
| 2139/2140 | G125K | ++++ | ++++ |
| 2141/2142 | K181R | ++++ | ++++ |
| 2143/2144 | V157F | ++++ | ++++ |
| 2145/2146 | C238K | ++++ | ++++ |
| 2147/2148 | M89Q | ++++ | ++++ |
| 2149/2150 | M89S | ++++ | ++++ |
| 2151/2152 | T330E | ++++ | +++ |
| 2153/2154 | V122C | ++++ | ++++ |
| 2155/2156 | M89Y | ++++ | ++++ |
| 2157/2158 | M89C | ++++ | ++++ |
| 2159/2160 | T212E | ++++ | ++++ |
| 2161/2162 | M89W | ++++ | +++ |
| 2163/2164 | V143S | ++++ | ++++ |
| 2165/2166 | M89V | ++++ | ++++ |
| 2167/2168 | A208P | ++++ | ++++ |
| 2169/2170 | T290D | ++++ | +++ |
| 2171/2172 | L161V | ++++ | ++++ |
| 2173/2174 | L161M | +++ | ++++ |
| 2175/2176 | L214C | +++ | +++ |
| 2177/2178 | K181N | +++ | ++++ |
| 2179/2180 | I84V | +++ | ++++ |
| 2181/2182 | F270L | +++ | +++ |
| 2183/2184 | S91A | +++ | +++ |
| 2185/2186 | G295V | +++ | +++ |
| 2187/2188 | V122W | +++ | ++ |
| 2189/2190 | L316Y | +++ | +++ |
| 2191/2192 | I286E | +++ | +++ |
| 2193/2194 | T301M | +++ | +++ |
| 2195/2196 | F170Y | +++ | +++ |
| 2197/2198 | K264A | +++ | +++ |
| 2199/2200 | V289D | +++ | +++ |
| 2201/2202 | V289T | +++ | +++ |
| 2203/2204 | T290P | +++ | +++ |
| 2205/2206 | L190I | +++ | +++ |
| 2207/2208 | L316I | +++ | +++ |
| 2209/2210 | L183V | +++ | +++ |
| 2211/2212 | D128E | +++ | +++ |
| 2213/2214 | V289E | +++ | +++ |
| 2215/2216 | S91E | +++ | +++ |
| 2217/2218 | P60V | +++ | +++ |
| 2219/2220 | A310M | +++ | +++ |
| 2221/2222 | V131T | +++ | +++ |
| 2223/2224 | V143F | +++ | +++ |
| 2225/2226 | T301V | +++ | +++ |
| 2227/2228 | A208H | +++ | +++ |
| 2229/2230 | M302S | +++ | +++ |
| 2231/2232 | P60R | +++ | +++ |
| 2233/2234 | V143E | +++ | +++ |
| 2235/2236 | V122Q | +++ | +++ |
| 2237/2238 | L267M | +++ | ++ |
| 2239/2240 | V157L | +++ | +++ |
| 2241/2242 | S288W | +++ | +++ |
| 2243/2244 | N185K | +++ | +++ |
| 2245/2246 | A291Q | +++ | +++ |
| 2247/2248 | K264E | +++ | +++ |
| 2249/2250 | F129W | +++ | +++ |
| 2251/2252 | S280L | +++ | +++ |
| 2253/2254 | S298R | +++ | +++ |
| 2255/2256 | G295K | +++ | +++ |
| 2257/2258 | V122T | +++ | +++ |
| 2259/2260 | L267I | +++ | +++ |
| 2261/2262 | M89G | +++ | +++ |
| 2263/2264 | I286V | +++ | +++ |
| 2265/2266 | N185F | +++ | +++ |
| 2267/2268 | G158R/A215G | +++ | +++ |
| 2269/2270 | G295T | +++ | +++ |
| 2271/2272 | I28A/V122G | +++ | ++++ |
| 2273/2274 | W276M | +++ | +++ |
| 2275/2276 | V274A | +++ | ++ |
| 2277/2278 | N185M | +++ | ++ |
| 2279/2280 | S271T | +++ | +++ |
| 2281/2282 | A215T | +++ | +++ |
| 2283/2284 | G295P | +++ | +++ |
| 2285/2286 | F294V | +++ | +++ |
| 2287/2288 | F270M | ++ | +++ |
| 2289/2290 | K264S | ++ | +++ |
| 2291/2292 | S106L | ++ | +++ |
| 2293/2294 | T301F | ++ | ++ |
| 2295/2296 | G295L | ++ | +++ |
| 2297/2298 | V122L | ++ | ++ |
| 2299/2300 | S106R | ++ | +++ |
| 2301/2302 | S280N | ++ | +++ |
| 2303/2304 | W276G | ++ | ++ |
| 2305/2306 | A310H | ++ | ++ |
| 2307/2308 | A208K | ++ | +++ |
| 2309/2310 | S106Q | ++ | +++ |
| 2311/2312 | I58M | ++ | ++ |
| 2313/2314 | G295Y | ++ | +++ |
| 2315/2316 | A291W | ++ | + |
| 2317/2318 | A208T | ++ | +++ |
| 2319/2320 | L135V | ++ | +++ |
| 2321/2322 | A45S | ++ | +++ |
| 2323/2324 | I28L | ++ | +++ |
| 2325/2326 | G299V | ++ | ++ |
| 2327/2328 | L214Y | ++ | ++ |
| 2329/2330 | G295Q | ++ | ++ |
| 2331/2332 | T290N | ++ | ++ |
| 2333/2334 | M302L | ++ | ++ |
| 2335/2336 | D315S | ++ | ++ |
| 2337/2338 | V143C | ++ | +++ |
| 2339/2340 | M89R | ++ | ++ |
| 2341/2342 | F204W | ++ | ++ |
| 2343/2344 | L332W | ++ | ++ |
| 2345/2346 | S169G | ++ | ++ |
| 2347/2348 | I305V | ++ | ++ |
| 2349/2350 | M89L | ++ | +++ |
| 2351/2352 | C154S | ++ | +++ |
| 2353/2354 | G295R | ++ | +++ |
| 2355/2356 | G158R | ++ | +++ |
| 2357/2358 | L135A | ++ | +++ |
| 2359/2360 | C238V | ++ | +++ |
| 2361/2362 | T290H | ++ | ++ |
| 2363/2364 | F294A | ++ | ++ |
| 2365/2366 | G299I | ++ | + |
| 2367/2368 | M89N | ++ | +++ |
| 2369/2370 | S288L | ++ | ++ |
| 2371/2372 | G295F | ++ | ++ |
| 2373/2374 | V297I | ++ | ++ |
| 2375/2376 | G125T | ++ | ++ |
| 2377/2378 | D315E | ++ | ++ |
| 2379/2380 | V95C | ++ | +++ |
| 2381/2382 | I337V | ++ | ++ |
| 2383/2384 | K264G | ++ | ++ |
| 2385/2386 | G295S | ++ | +++ |
| 2387/2388 | V289P | ++ | ++ |
| 2389/2390 | T212I | ++ | +++ |
| 2391/2392 | W276A | ++ | ++ |
| 2393/2394 | P60C | ++ | +++ |
| 2395/2396 | L214A | ++ | + |
| 2397/2398 | A324D | ++ | ++ |
| 2399/2400 | N185C | ++ | ++ |
| 2401/2402 | M302C | ++ | ++ |
| 2403/2404 | A324M | ++ | + |
| 2405/2406 | T301L | ++ | ++ |
| 2407/2408 | A310N | ++ | ++ |
| 2409/2410 | S298C | ++ | ++ |
| 2411/2412 | G295M | ++ | ++ |
| 2413/2414 | G299T | ++ | +++ |
| 2415/2416 | V150L | ++ | ++ |
| 2417/2418 | C238S | ++ | ++ |
| 2419/2420 | A291T | ++ | ++ |
| 2421/2422 | L135M | ++ | ++ |
| 2423/2424 | D179E | ++ | ++ |
| 2425/2426 | L283C | ++ | ++ |
| 2427/2428 | A310S | ++ | ++ |
| 2429/2430 | P206L | ++ | ++ |
| 2431/2432 | A310Q | ++ | ++ |
| 2433/2434 | S106V | ++ | ++ |
| 2435/2436 | K181S | ++ | ++ |
| 2437/2438 | S298K | ++ | ++ |
| 2439/2440 | G299Q | ++ | ++ |
| 2441/2442 | G295C | ++ | + |
| 2443/2444 | V143M | ++ | ++ |
| 2445/2446 | P60S | ++ | ++ |
| 2447/2448 | L214H | ++ | ++ |
| 2449/2450 | T212L | ++ | ++ |
| 2451/2452 | I236V | ++ | ++ |
| 2453/2454 | L266C | ++ | ++ |
| 2455/2456 | V289A | ++ | ++ |
| 2457/2458 | T330A | ++ | +++ |
| 2459/2460 | G213N | ++ | + |
| 2461/2462 | M302A | ++ | ++ |
| 2463/2464 | F294S | ++ | ++ |
| 2465/2466 | T336I | ++ | ++ |
| 2467/2468 | N178V | ++ | ++ |
| 2469/2470 | A324G | ++ | + |
| 2471/2472 | V297L | ++ | ++ |
| 2473/2474 | L99I | ++ | ++ |
| 2475/2476 | V184L | ++ | ++ |
| 2477/2478 | S169N | ++ | ++ |
| 2479/2480 | T212G | ++ | ++ |
| 2481/2482 | V282A | ++ | ++ |
| 2483/2484 | S271G | ++ | ++ |
| 2485/2486 | S106M | ++ | ++ |
| 2487/2488 | S271A | ++ | + |
| 2489/2490 | P60D | ++ | ++ |
| 2491/2492 | M89T | ++ | ++ |
| 2493/2494 | M302I | ++ | + |
| 2495/2496 | F270V | ++ | +++ |
| 2497/2498 | I57V | ++ | + |
| 2499/2500 | V143H | ++ | ++ |
| 2501/2502 | P177K | ++ | ++ |
| 2503/2504 | A291L | ++ | + |
| 2505/2506 | L214Q | ++ | + |
| 2507/2508 | T301I | ++ | ++ |
| 2509/2510 | S271E | ++ | + |
| 2511/2512 | I109L | ++ | + |
| 2513/2514 | V191C | ++ | + |
| 2515/2516 | G213T | ++ | ++ |
| 2517/2518 | V107S | ++ | + |
| 2519/2520 | S106W | ++ | ++ |
| 2521/2522 | N185G | ++ | + |
| 2523/2524 | A310R | ++ | + |
| 2525/2526 | V289I | ++ | ++ |
| 2527/2528 | V282M | ++ | + |
| 2529/2530 | T336L | ++ | + |
| 2531/2532 | L332H | ++ | ++ |
| 2533/2534 | A291H | ++ | + |
| 2535/2536 | S298V | ++ | + |
| 2537/2538 | L135H | ++ | ++ |
| 2539/2540 | S280W | ++ | ++ |
| 2541/2542 | A310L | ++ | + |
| 2543/2544 | L266I | ++ | + |
| 2545/2546 | L135Q | ++ | ++ |
| 2547/2548 | G158S | ++ | + |
| 2549/2550 | S288R | ++ | + |
| 2551/2552 | K160R | ++ | + |
| 2553/2554 | A310T | ++ | + |
| 2555/2556 | I286F | ++ | ++ |
| 2557/2558 | W276V | ++ | + |
| 2559/2560 | A291R | + | ++ |
| 2561/2562 | T330G | + | ++++ |
| 2563/2564 | F129L | + | + |
| 2565/2566 | V143T | + | + |
| 2567/2568 | T212K | + | + |
| 2569/2570 | A208I | + | + |
| 2571/2572 | V297A | + | ++ |
| 2573/2574 | I286D | + | + |
| 2575/2576 | V157Q | + | + |
| 2577/2578 | T301A | + | + |
| 2579/2580 | Y144L | + | ++ |
| 2581/2582 | S106G | + | + |
| 2583/2584 | L283V | + | + |
| 2585/2586 | V56L | + | + |
| 2587/2588 | A291S | + | + |
| 2589/2590 | K264V | + | + |
| 2591/2592 | A215G | + | ++ |
| 2593/2594 | F170H | + | + |
| 2595/2596 | F294Y | + | + |
| 2597/2598 | L159F | + | + |
| 2599/2600 | I286M | + | + |
| 2601/2602 | V95I | + | + |
| 2603/2604 | W341A | + | ++ |
| 2605/2606 | V325L | + | + |
| 2607/2608 | L214K | + | + |
| 2609/2610 | T336M | + | + |
| 2611/2612 | S91V | + | ++ |
| 2613/2614 | I337A | + | + |
| 2615/2616 | N185T | + | + |
| 2617/2618 | N185S | + | + |
| 2619/2620 | N185V | + | + |

Levels of increased activity were determined for Activity FIOP on Antisense Strand relative to SEQ ID NO: 1866 and are defined as follows: “+” > 1.0, “++” > 1.4, “+++” > 1.8, “++++” > 2.2. Levels of increased activity were determined for Activity FIOP on Sense Strand relative to SEQ ID NO: 1866 and are defined as follows: “+” > 1.0, “++” > 1.6, “+++” > 2.0, “++++” > 3.0.

### Example 21

### Improvements Over SEQ ID NO: 2062 in RNA Ligase Activity

SEQ ID NO: 2062 was selected as the parent enzyme in an RNA ligation assay. Libraries of engineered genes were produced using known techniques (e.g., saturation mutagenesis, recombination of previously identified beneficial mutations). The polypeptides encoded by each gene were produced in high throughput (HTP) as described in Example 1, and the cell paste was generated as described in Example 2. To prepare lysate, 600 ul of 0.1 mg/mL lysozyme in 50 mM Tris-HCl buffer at pH 7.5 was added to the cell paste of each sample. After thorough resuspension at room temperature on a plate mixer for 1 hour, the cells were centrifuged for 20 min at 4000 rpm and 4° C. The supernatant was incubated at 40° C. for 1 hour in a PCR thermocycler. The samples were then centrifuged for 10 min at 4000 rpm and 4° C., and the clear supernatants were collected for ligation reactions.

The oligonucleotide mix for Reaction 6 in Example 4 were used as substrates in the ligation reaction. The lysate was screened in a 100 nanoliter reaction that comprised 50 mM Tris-HCl buffer at pH 7.5, 20 mM MgCl₂, 1 mM dithiothreitol (DTT), 8 mM adenosine triphosphate (ATP), and 1.25 mM of an equimolar mixture of the oligonucleotide fragments compromising Reaction 6. Nanoliter scale liquid transfers were carried out by Echo Acoustic Liquid Handler. The reactions were mixed by vortex, incubated for 2 hr at 33° C. in a PCR thermocycler, and quenched and diluted for analytical analysis by CE as described in Example 4. Using the peak area from CE analytical method, the % conversions of sense strand and antisense strand for each sample were calculated as shown below:

$$\% conversion = \frac{area under the curve for the product peak}{\begin{matrix}Total area under the curve for all \\ products and remaining fragments\end{matrix}} * 100$$

Activity relative to SEQ ID NO: 2062 (Activity FIOP) was calculated as the % conversion by the variant over the % conversion by SEQ ID NO: 2062. The result is shown in Table 21.1.

TABLE 21.1
RNA ligase 2 activity relative to SEQ ID NO: 2062

| | Amino Acid | Activity FIOP on | Activity FIOP on |
|---|---|---|---|
| SEQ | Differences | Antisense Strand | Sense Strand |
| ID NO: | (Relative to SEQ | relative to SEQ | relative to SEQ |
| (nt/aa) | ID NO: 2062) | ID NO: 2062 | ID NO: 2062 |
| 2621/2622 | I83A/I84V/T330E | ++++ | +++ |
| 2623/2624 | S91A/F270L/T290D | ++++ | ++++ |
| 2625/2626 | I83A/K181P | ++++ | ++++ |
| 2627/2628 | L161V/T290D/T330E | ++++ | +++ |
| 2629/2630 | K181R/T330E | ++++ | +++ |
| 2631/2632 | L161V/F270L/T330E | ++++ | +++ |
| 2633/2634 | L161M/F270L/T330E | ++++ | +++ |
| 2635/2636 | F270L/T290D | ++++ | +++ |
| 2637/2638 | I83A/I84V/F270L/F318S/T330E | ++++ | ++++ |
| 2639/2640 | V64Q/I83A/I84V/L161M/D192E/T290D/F318S | ++++ | ++++ |
| 2641/2642 | S91A/L161V/T290D/F318S/T330E | ++++ | +++ |
| 2643/2644 | L161V/T330E | ++++ | +++ |
| 2645/2646 | L161V/L214C/F270L/T290D/T330E | +++ | +++ |
| 2647/2648 | D192E/F270L/F318S | +++ | ++++ |
| 2649/2650 | S91A/L161M/D192E/T330E | +++ | +++ |
| 2651/2652 | L161V/F270L | +++ | ++++ |
| 2653/2654 | K181P/T330E | +++ | ++++ |
| 2655/2656 | K181P/F270L/T330E | +++ | ++ |
| 2657/2658 | S91A/K181R/D192E/T290D | +++ | +++ |
| 2659/2660 | L161M/T290D | +++ | ++++ |
| 2661/2662 | I83A/L161M/F270L | +++ | +++ |
| 2663/2664 | I83A/D192E/F318S | +++ | +++ |
| 2665/2666 | L214C/T330E | +++ | ++ |
| 2667/2668 | I83A/L161M/T290D/T330E | +++ | ++ |
| 2669/2670 | V64Q/I83A/L161M/T290D/T330E | +++ | +++ |
| 2671/2672 | I83A/L161M/D192E/L214C/F318S | +++ | ++++ |
| 2673/2674 | V122F/V143S/T212E/G295V | +++ | + |
| 2675/2676 | L161V/D192E/F270L/T290D | +++ | ++++ |
| 2677/2678 | K181P/T290D | +++ | +++ |
| 2679/2680 | S91A/L161V/L214C/F270L/T290D/F318S | +++ | +++ |
| 2681/2682 | S91A/D192E/L214C/F270L | ++ | +++ |
| 2683/2684 | L214C/T290D | ++ | ++ |
| 2685/2686 | V64Q/I83A/L161M/F270L/F318S/T330E | ++ | +++ |
| 2687/2688 | S91A/L161M/K181P/T330E | ++ | +++ |
| 2689/2690 | L161M/F318S/T330E | ++ | +++ |
| 2691/2692 | S91A/F270L/T290D/T330E | ++ | ++ |
| 2693/2694 | V64Q/K181P/T290D/F318S | ++ | +++ |
| 2695/2696 | K181R/F270L/T290D/F318S | ++ | +++ |
| 2697/2698 | V64Q/I83A/L161V/D192E/F270L/T290D/F318S/T330E | ++ | ++++ |
| 2699/2700 | V64Q/I83A/L161M/L214C/T290D/F318S | ++ | ++ |
| 2701/2702 | V122F/D192E/T212E | ++ | ++ |
| 2703/2704 | V64Q/I83A/S91A/D192E/L214C/T330E | ++ | ++ |
| 2705/2706 | I83A/S91A/L161M/K181R/T290D/F318S | ++ | ++++ |
| 2707/2708 | S91A/L161V/D192E/L214C/F270L/T290D | ++ | ++ |
| 2709/2710 | S91A/L161V/K181P/T290D/T330E | ++ | +++ |
| 2711/2712 | I83A/S91A/G125K/L161M/F270L | ++ | + |
| 2713/2714 | L161M/T290D/T330E | ++ | ++ |
| 2715/2716 | I28V/T212E | ++ | + |
| 2717/2718 | V122F/T212E | ++ | |
| 2719/2720 | I28V/V64Q/I83A/V122F/V143S/D192E/T212E/G295V/F318S | ++ | + |
| 2721/2722 | V122F/G295V | ++ | + |
| 2723/2724 | S91A/L161V/D192E/F318S | ++ | ++ |
| 2725/2726 | I28V/V122F/V143S/T212E | ++ | |
| 2727/2728 | V64Q/L161V/T290D | ++ | +++ |
| 2729/2730 | V64Q/V122F/A208P/F318S | ++ | |
| 2731/2732 | V64Q/I83A/D192E/T290D/F318S | + | +++ |
| 2733/2734 | V64Q/I83A/L161V/T290D | + | +++ |
| 2735/2736 | K181R/D192E/F318S | + | +++ |
| 2737/2738 | I83A/L161V/D192E/F318S | + | ++ |
| 2739/2740 | L214C/F270L | + | ++ |
| 2741/2742 | I83A/V122F | + | |
| 2743/2744 | D192E/T212E | + | + |
| 2745/2746 | I28V/V122F | + | |

Levels of increased activity were determined for Activity FIOP on Antisense Strand relative to SEQ ID NO: 2062 and are defined as follows: “+” > 1.0, “++” > 1.55, “+++” > 1.75, “++++” > 2.0. Levels of increased activity were determined for Activity FIOP on Sense Strand relative to SEQ ID NO: 2062 and are defined as follows: “+” > 1.0, “++” > 1.1, “+++” > 1.2, “++++” > 1.3.

### Example 22

### Improvements Over SEQ ID NO: 2674 in RNA Ligase Activity

SEQ ID NO: 2674 was selected as the parent enzyme in an RNA ligation assay. Libraries of engineered genes were produced using known techniques (e.g., saturation mutagenesis, recombination of previously identified beneficial mutations). The polypeptides encoded by each gene were produced in high throughput (HTP) as described in Example 1, and the cell paste was generated as described in Example 2. To prepare lysate, 600 ul of 0.1 mg/mL lysozyme in 50 mM Tris-HCl buffer at pH 7.5 was added to the cell paste of each sample. After thorough resuspension at room temperature on a plate mixer for 1 hour, the cells were centrifuged for 20 min at 4000 rpm and 4° C. The supernatant was diluted 2-fold in 50 mM Tris-HCl buffer at pH 7.5 and incubated at 40° C. for 1 hour in a PCR thermocycler. The samples were then centrifuged for 10 min at 4000 rpm and 4° C., and the clear supernatants were collected for ligation reactions.

The oligonucleotide mix for Reaction 4 in Example 4 were used as substrates in the ligation reaction. The lysate was screened in a 100 nanoliter reaction that comprised 50 mM Tris-HCl buffer at pH 7.5, 20 mM MgCl₂, 1 mM dithiothreitol (DTT), 8 mM adenosine triphosphate (ATP), and 1.25 mM of an equimolar mixture of the oligonucleotide fragments compromising Reaction 4. Nanoliter scale liquid transfers were carried out by Echo Acoustic Liquid Handler. The reactions were mixed by vortex, incubated for 2 hr at 33° C. in a PCR thermocycler, and quenched and diluted for analytical analysis by CE as described in Example 4. Using the peak area from CE analytical method, the % conversions of sense strand and antisense strand for each sample were calculated as shown below:

$$\% conversion = \frac{area under the curve for the product peak}{\begin{matrix}Total area under the curve for all \\ products and remaining fragments\end{matrix}} * 100$$

Activity relative to SEQ ID NO: 2674 (Activity FIOP) was calculated as the % conversion by the variant over the % conversion by SEQ ID NO: 2674. The result is shown in Table 22.1.

TABLE 22.1
RNA ligase 2 activity relative to SEQ ID NO: 2674

| | Amino Acid | Activity FIOP on | Activity FIOP on |
|---|---|---|---|
| SEQ | Differences | Antisense Strand | Sense Strand |
| ID NO: | (Relative to SEQ | relative to SEQ | relative to SEQ |
| (nt/aa) | ID NO: 2674) | ID NO: 2674 | ID NO: 2674 |
| 2747/2748 | I28L/A291Q/V295G/S298V/D315E | ++ | ++++ |
| 2749/2750 | I28L/V95I/A291S/V295G/S298V/D315E | ++ | ++++ |
| 2751/2752 | S143T/V295G/S298V/D315E | ++ | ++++ |
| 2753/2754 | A291Q/V295G/S298V | + | ++++ |
| 2755/2756 | I28L/S143V/V295G/S298V | ++ | +++ |
| 2757/2758 | I28L/A291S/V295G/S298V | + | +++ |
| 2759/2760 | S143V/N185V/A291Q/V295G/S298V | ++++ | ++ |
| 2761/2762 | I28L/S143V/N185V/A291Q/S298V/T336I | ++++ | ++ |
| 2763/2764 | I28L/S298V | ++++ | + |
| 2765/2766 | S280N/V295G | +++ | |
| 2767/2768 | S143V/S298V/T336I | ++++ | |
| 2769/2770 | S143V/A291S/V295G/S298V | + | +++ |
| 2771/2772 | A291Q/V295G/S298V/D315E/T336I | ++ | ++ |
| 2773/2774 | S143V/V295G/S298V | + | ++ |
| 2775/2776 | I28L/S143T/V295G/S298V/T336I | + | ++ |
| 2777/2778 | V95I/S143T/V295G/S298V | + | ++ |
| 2779/2780 | S143V/S280N/T290N | | + |
| 2781/2782 | S143T/A291S/S298V | + | + |

Levels of increased activity were determined for Activity FIOP on Antisense Strand relative to SEQ ID NO: 2674 and are defined as follows: “+” > 1.0, “++” > 1.025, “+++” > 1.1, “++++” > 1.3. Levels of increased activity were determined for Activity FIOP on Sense Strand relative to SEQ ID NO: 2674 and are defined as follows: “+” > 1.0, “++” > 1.1, “+++” > 1.3, “++++” > 2.0.

### Example 23

### Improvements Over SEQ ID NO: 2754 in RNA Ligase Activity

SEQ ID NO: 2754 was selected as the parent enzyme in an RNA ligation assay. Libraries of engineered genes were produced using known techniques (e.g., saturation mutagenesis, recombination of previously identified beneficial mutations). The polypeptides encoded by each gene were produced in high throughput (HTP) as described in Example 1, and the cell paste was generated as described in Example 2. To prepare lysate, 600 ul of 0.1 mg/mL lysozyme in 50 mM Tris-HCl buffer at pH 7.5 was added to the cell paste of each sample. After thorough resuspension at room temperature on a plate mixer for 1 hour, the cells were centrifuged for 20 min at 4000 rpm and 4° C. The supernatant was diluted 4-fold in 50 mM Tris-HCl buffer at pH 7.5 and incubated at 40° C. for 1 hour in a PCR thermocycler. The samples were then centrifuged for 10 min at 4000 rpm and 4° C., and the clear supernatants were collected for ligation reactions.

The oligonucleotide mix for Reaction 7 in Example 4 were used as substrates in the ligation reaction. The lysate was screened in a 100 nanoliter reaction that comprised 50 mM Tris-HCl buffer at pH 7.5, 20 mM MgCl₂, 1 mM dithiothreitol (DTT), 8 mM adenosine triphosphate (ATP), and 1.25 mM of an equimolar mixture of the oligonucleotide fragments compromising Reaction 7. Nanoliter scale liquid transfers were carried out by Echo Acoustic Liquid Handler. The reactions were mixed by vortex, incubated for 2 hr at 33° C. in a PCR thermocycler, and quenched and diluted for analytical analysis by CE as described in Example 4. Using the peak area from CE analytical method, the % conversions of sense strand and antisense strand for each sample were calculated as shown below:

$$\% conversion = \frac{area under the curve for the product peak}{\begin{matrix}Total area under the curve for all \\ products and remaining fragments\end{matrix}} * 100$$

Activity relative to SEQ ID NO: 2754 (Activity FIOP) was calculated as the % conversion by the variant over the % conversion by SEQ ID NO: 2754. The result is shown in Table 23.1.

TABLE 23.1
RNA ligase 2 activity relative to SEQ ID NO: 2754

| | Amino Acid | Activity FIOP on | Activity FIOP on |
|---|---|---|---|
| SEQ | Differences | Antisense Strand | Sense Strand |
| ID NO: | (Relative to SEQ | relative to SEQ | relative to SEQ |
| (nt/aa) | ID NO: 2754) | ID NO: 2754 | ID NO: 2754 |
| 2783/2784 | S106R/K264E/S280L | ++++ | ++++ |
| 2785/2786 | S280L/T330A | +++ | ++++ |
| 2787/2788 | I28L/S106M/K264S/Q291S | ++++ | +++ |
| 2789/2790 | I28L/K264E/S280L/T330A | ++ | ++++ |
| 2791/2792 | I57V/P60R/L135V/K264S/Q291S | ++++ | ++ |
| 2793/2794 | I28L/L135V/K264S/S280L | +++ | ++ |
| 2795/2796 | S106L/K264E/S280L/Q291S | ++++ | ++ |
| 2797/2798 | I28L/S106L/K264S/S280L | +++ | ++ |
| 2799/2800 | S280L/G299I | | ++++ |
| 2801/2802 | I28L/I57V/M89G/S106L/K264S/S280L/Q291S | ++++ | + |
| 2803/2804 | K264S | ++ | ++ |
| 2805/2806 | N185S/Q291S | +++ | ++ |
| 2807/2808 | I28L/S106M/G299V | | ++++ |
| 2809/2810 | I28L/L214Y/K264S/G299V | | +++ |
| 2811/2812 | K264E/T330A | | + |
| 2813/2814 | I28V/M89N/L214Q/Q291S | ++ | |
| 2815/2816 | I28V/N185K/S271G/A310H | | + |
| 2817/2818 | F122T/S271G | | ++ |
| 2819/2820 | M89N/S106M/L135V/K264E/S280L/T330A | | + |

Levels of increased activity were determined for Activity FIOP on Antisense Strand relative to SEQ ID NO: 2754 and are defined as follows: “+” > 1.0, “++” > 1.2, “+++” > 1.4, “++++” > 1.7. Levels of increased activity were determined for Activity FIOP on Sense Strand relative to SEQ ID NO: 2754 and are defined as follows: “+” > 1.0, “++” > 1.3, “+++” > 2.0, “++++” > 2.5.

### Example 24

### Improvements Over SEQ ID NO: 2674 in RNA Ligase Activity

SEQ ID NO: 2674 was selected as the parent enzyme in an RNA ligation assay. Libraries of engineered genes were produced using known techniques (e.g., saturation mutagenesis, recombination of previously identified beneficial mutations). The polypeptides encoded by each gene were produced in high throughput (HTP) as described in Example 1, and the cell paste was generated as described in Example 2. To prepare lysate, 600 ul of 0.1 mg/mL lysozyme in 50 mM Tris-HCl buffer at pH 7.5 was added to the cell paste of each sample. After thorough resuspension at room temperature on a plate mixer for 1 hour, the cells were centrifuged for 20 min at 4000 rpm and 4° C. The supernatant was diluted 4-fold in 50 mM Tris-HCl buffer at pH 7.5 and incubated at 40° C. for 1 hour in a PCR thermocycler. The samples were then centrifuged for 10 min at 4000 rpm and 4° C., and the clear supernatants were collected for ligation reactions.

The oligonucleotide mix for Reaction 6 in Example 4 were used as substrates in the ligation reaction. The lysate was screened in a 100 nanoliter reaction that comprised 50 mM Tris-HCl buffer at pH 7.5, 20 mM MgCl₂, 1 mM dithiothreitol (DTT), 8 mM adenosine triphosphate (ATP), and 1.25 mM of an equimolar mixture of the oligonucleotide fragments compromising Reaction 6. Nanoliter scale liquid transfers were carried out by Echo Acoustic Liquid Handler. The reactions were mixed by vortex, incubated for 2 hr at 33° C. in a PCR thermocycler, and quenched and diluted for analytical analysis by CE as described in Example 4. Using the peak area from CE analytical method, the % conversions of sense strand and antisense strand for each sample were calculated as shown below:

$$\% conversion = \frac{area under the curve for the product peak}{\begin{matrix}Total area under the curve for all \\ products and remaining fragments\end{matrix}} * 100$$

Activity relative to SEQ ID NO: 2674 (Activity FIOP) was calculated as the % conversion by the variant over the % conversion by SEQ ID NO: 2674. The result is shown in Table 24.1.

TABLE 24.1
RNA ligase 2 activity relative to SEQ ID NO: 2674

| | Amino Acid | Activity FIOP on | Activity FIOP on |
|---|---|---|---|
| SEQ | Differences | Antisense Strand | Sense Strand |
| ID NO: | (Relative to SEQ | relative to SEQ | relative to SEQ |
| (nt/aa) | ID NO: 2674) | ID NO: 2674 | ID NO: 2674 |
| 2821/2822 | A208T | + | |
| 2823/2824 | A25G | + | + |
| 2825/2826 | D90E | ++ | |
| 2827/2828 | E141D | +++ | ++++ |
| 2829/2830 | E258D | +++ | ++++ |
| 2831/2832 | F248G | ++++ | |
| 2833/2834 | F248K | ++++ | |
| 2835/2836 | F318E | ++++ | ++++ |
| 2837/2838 | F318S | +++ | + |
| 2839/2840 | G117Q | + | |
| 2841/2842 | G299K | | ++ |
| 2843/2844 | G33I | + | |
| 2845/2846 | G33T | +++ | + |
| 2847/2848 | G80E | ++ | |
| 2849/2850 | I253W | ++++ | |
| 2851/2852 | I83E | ++ | + |
| 2853/2854 | I83T | | + |
| 2855/2856 | K254Q | ++ | |
| 2857/2858 | K254V | ++ | |
| 2859/2860 | K256A | + | |
| 2861/2862 | K256G | ++++ | |
| 2863/2864 | K256M | ++++ | + |
| 2865/2866 | K256W | +++ | |
| 2867/2868 | K30L | +++ | |
| 2869/2870 | K30V | ++++ | |
| 2871/2872 | K30W | ++++ | ++ |
| 2873/2874 | K327E | +++ | ++++ |
| 2875/2876 | L73Q | +++ | + |
| 2877/2878 | L73W | + | |
| 2879/2880 | N100A | +++ | ++ |
| 2881/2882 | N100G | + | |
| 2883/2884 | N100H | ++ | +++ |
| 2885/2886 | N100S | +++ | + |
| 2887/2888 | N97A | +++ | + |
| 2889/2890 | Q119L | | ++++ |
| 2891/2892 | R265A | + | |
| 2893/2894 | R296E | ++ | |
| 2895/2896 | R296L | | + |
| 2897/2898 | R296Q | | + |
| 2899/2900 | R32E | +++ | |
| 2901/2902 | T156Q | ++ | |
| 2903/2904 | T40I | + | |
| 2905/2906 | V105L | ++ | ++++ |
| 2907/2908 | V107M | ++ | |
| 2909/2910 | V41E/A114E | ++ | + |
| 2911/2912 | V41I | +++ | + |
| 2913/2914 | V64A | + | |

Levels of increased activity were determined for Activity FIOP on Antisense Strand relative to SEQ ID NO: 2674 and are defined as follows: “+” > 1.0, “++” > 1.04, “+++” > 1.12, “++++” > 1.3. Levels of increased activity were determined for Activity FIOP on Sense Strand relative to SEQ ID NO: 2674 and are defined as follows: “+” > 1.0, “++” > 1.04, “+++” > 1.07, “++++” > 1.1.

### Example 25

### Improvements Over SEQ ID NO: 2674 in RNA Ligase Thermostability

The RNA ligase of SEQ ID NO: 2674 was selected as the parent enzyme in an RNA ligation assay. Libraries of engineered genes were produced using known techniques (e.g., saturation mutagenesis, recombination of previously identified beneficial mutations). The polypeptides encoded by each gene were produced in high throughput (HTP) as described in Example 1, and the cell paste was generated as described in Example 2. To prepare lysate, 600 ul of 0.1 mg/mL lysozyme in 50 mM Tris-HCl buffer at pH 7.5 was added to the cell paste of each sample. After thorough resuspension at room temperature on a plate mixer for 30 min, the cells were diluted 2-fold in 50 mM Tris-HCl buffer at pH 7.5 then preincubated at 51.5° C. for 1 hour in a PCR thermocycler. The samples were then centrifuged for 10 min at 4000 rpm and 4° C. to remove the precipitated proteins, and the remainder clear supernatants were collected for ligation reactions.

The oligonucleotide mix for Reaction 3 in Example 4 were used as substrates in the ligation reaction. The lysate was screened in a 100 nanoliter reaction that comprised 50 mM Tris-HCl buffer at pH 7.5, 20 mM MgCl₂, 1 mM dithiothreitol (DTT), 8 mM adenosine triphosphate (ATP), and 1.6 mM of an equimolar mixture of the oligonucleotide fragments compromising Reaction 3. Nanoliter scale liquid transfers were carried out by Echo Acoustic Liquid Handler. The reactions were mixed by vortex, incubated for 2 hr at 33° C. in a PCR thermocycler, and quenched and diluted for analytical analysis by CE as described in Example 4. Using the peak area from CE analytical method, the % conversion for each sample was calculated as shown below:

$$\% conversion = \frac{area under the curve for the product peak}{\begin{matrix}Total area under the curve for all \\ products and remaining fragments\end{matrix}} * 100$$

Activity relative to SEQ ID NO: 2674 (Activity FIOP) was calculated as the % conversion by the variant over the % conversion by SEQ ID NO: 2674. The result is shown in Table 25.1.

TABLE 25.1
RNA ligase 2 activity relative to SEQ ID NO:
2674 after 51.5° C. heat challenge for 60 min

| | Amino Acid | Activity FIOP on | Activity FIOP on |
|---|---|---|---|
| SEQ | Differences | Antisense Strand | Sense Strand |
| ID NO: | (Relative to | relative to SEQ | relative to SEQ |
| (nt/aa) | SEQ ID NO: 2674) | ID NO: 2674 | ID NO: 2674 |
| 2915/2916 | A162C | + | |
| 2917/2918 | A208L | ++ | ++ |
| 2919/2920 | A25D | +++ | + |
| 2921/2922 | A25F | +++ | ++++ |
| 2923/2924 | A25T | ++ | +++ |
| 2925/2926 | D103V | + | ++ |
| 2927/2928 | E346S | +++ | +++ |
| 2929/2930 | E42T | + | + |
| 2931/2932 | E74D | +++ | ++ |
| 2933/2934 | F113M | ++ | |
| 2935/2936 | F13W | ++ | +++ |
| 2937/2938 | F228D | +++ | + |
| 2939/2940 | F228L | ++ | |
| 2941/2942 | F318A | ++ | + |
| 2943/2944 | F318G | +++ | ++++ |
| 2945/2946 | G121N | + | |
| 2947/2948 | G299E | ++++ | |
| 2949/2950 | G299R | + | ++ |
| 2951/2952 | G33L | +++ | ++++ |
| 2953/2954 | H342S | ++ | +++ |
| 2955/2956 | I253L | +++ | ++ |
| 2957/2958 | K127P | ++ | + |
| 2959/2960 | K254E | ++++ | +++ |
| 2961/2962 | K254N | +++ | +++ |
| 2963/2964 | K292N | ++ | +++ |
| 2965/2966 | K292S | +++ | ++++ |
| 2967/2968 | K30A | ++++ | ++++ |
| 2969/2970 | K30T | +++ | ++++ |
| 2971/2972 | K67R | + | ++ |
| 2973/2974 | K86L | + | ++ |
| 2975/2976 | L165M | ++++ | ++ |
| 2977/2978 | L173M | ++++ | |
| 2979/2980 | L300A | ++ | ++ |
| 2981/2982 | L317E | ++ | ++ |
| 2983/2984 | L317Q | ++++ | ++++ |
| 2985/2986 | L317V | ++ | +++ |
| 2987/2988 | L326C | + | |
| 2989/2990 | N11G | +++ | +++ |
| 2991/2992 | N229W | + | |
| 2993/2994 | N97E | +++ | +++ |
| 2995/2996 | P71T | ++ | +++ |
| 2997/2998 | S17P | ++ | |
| 2999/3000 | S210V | +++ | ++++ |
| 3001/3002 | S241E | ++ | + |
| 3003/3004 | S271G | ++ | ++ |
| 3005/3006 | T40P | ++ | +++ |
| 3007/3008 | T40S | +++ | +++ |
| 3009/3010 | V56I | ++ | +++ |
| 3011/3012 | V95A | +++ | +++ |

Levels of increased activity were determined for Activity FIOP on Antisense Strand relative to SEQ ID NO: 2674 and are defined as follows: “+” > 1.0, “++” > 1.7, “+++” > 3.0, “++++” > 8.0. Levels of increased activity were determined for Activity FIOP on Sense Strand relative to SEQ ID NO: 2674 and are defined as follows: “+” > 1.0, “++” > 1.4, “+++” > 3.5, “++++” > 8.0.

### Example 26

### Improvements Over SEQ ID NO: 2786 in RNA Ligase Activity

SEQ ID NO: 2786 was selected as the parent enzyme in an RNA ligation assay. Libraries of engineered genes were produced using known techniques (e.g., saturation mutagenesis, recombination of previously identified beneficial mutations). The polypeptides encoded by each gene were produced in high throughput (HTP) as described in Example 1, and the cell paste was generated as described in Example 2. To prepare lysate, 600 ul of 0.1 mg/mL lysozyme in 50 mM Tris-HCl buffer at pH 7.5 was added to the cell paste of each sample. After thorough resuspension at room temperature on a plate mixer for 1 hour, the cells were centrifuged for 20 min at 4000 rpm and 4° C. The supernatant was diluted 2-fold in 50 mM Tris-HCl buffer at pH 7.5 and incubated at 40° C. for 1 hour in a PCR thermocycler. The samples were then centrifuged for 10 min at 4000 rpm and 4° C., and the clear supernatants were collected for ligation reactions.

The oligonucleotide mix for Reaction 6 in Example 4 were used as substrates in the ligation reaction. The lysate was screened in a 100 nanoliter reaction that comprised 50 mM Tris-HCl buffer at pH 7.5, 20 mM MgCl₂, 1 mM dithiothreitol (DTT), 8 mM adenosine triphosphate (ATP), and 1.25 mM of an equimolar mixture of the oligonucleotide fragments compromising Reaction 6. Nanoliter scale liquid transfers were carried out by Echo Acoustic Liquid Handler. The reactions were mixed by vortex, incubated for 2 hr at 33° C. in a PCR thermocycler, and quenched and diluted for analytical analysis by CE as described in Example 4. Using the peak area from CE analytical method, the % conversions of sense strand and antisense strand for each sample were calculated as shown below:

$$\% conversion = \frac{area under the curve for the product peak}{\begin{matrix}Total area under the curve for all \\ products and remaining fragments\end{matrix}} * 100$$

Activity relative to SEQ ID NO: 2786 (Activity FIOP) was calculated as the % conversion by the variant over the % conversion by SEQ ID NO: 2786. The result is shown in Table 26.1.

TABLE 26.1
RNA ligase 2 activity relative to SEQ ID NO: 2786

| | Amino Acid | Activity FIOP on | Activity FIOP on |
|---|---|---|---|
| SEQ | Differences | Antisense Strand | Sense Strand |
| ID NO: | (Relative to SEQ | relative to SEQ | relative to SEQ |
| (nt/aa) | ID NO: 2786) | ID NO: 2786 | ID NO: 2786 |
| 3013/3014 | A25D/F228L/F248G/K264S | ++++ | |
| 3015/3016 | R32E/F113M/F228L/F248G | ++++ | |
| 3017/3018 | A25D/R32E/F113L/S241E/F248G/K264S/G299E | ++++ | |
| 3019/3020 | A25D/F113M/F248G/K264E/F318E | ++++ | |
| 3021/3022 | A25D/K127P/F228L/K264S/G299E/F318E | ++++ | |
| 3023/3024 | A25D/F113M/F228L/F248G/K327E | +++ | |
| 3025/3026 | F228L/S241E/K264E | +++ | ++++ |
| 3027/3028 | K264S/G299E/F318E/K327E | +++ | |
| 3029/3030 | A25D/F113M/S241E/F248G/K264S/F318E | +++ | |
| 3031/3032 | A25D/K127P/F248G/G299E/F318E | +++ | |
| 3033/3034 | F113M/K127P/F228L/K264E/G299E | +++ | |
| 3035/3036 | A25D/K127P | +++ | +++ |
| 3037/3038 | A25D/F228D/F248G/K264E/K327E | +++ | |
| 3039/3040 | A25D/F113M/K264E/G299E | ++ | |
| 3041/3042 | R32E/F113M/K264S/F318E | ++ | +++ |
| 3043/3044 | A25D/R32E/S241E/K264S/K327E | ++ | |
| 3045/3046 | F113M/K264E/K327E | ++ | |
| 3047/3048 | A25D/K127P/F228D/F248G/F318E | ++ | |
| 3049/3050 | K327E | + | |
| 3051/3052 | R32E/F113M/K254E/K264E | + | |
| 3053/3054 | A25D/F228L/S241E/K264E | + | + |
| 3055/3056 | A25D/F113M/F228L/S241E/F318E | + | ++ |
| 3057/3058 | F113M/K327E | + | |

Levels of increased activity were determined for Activity FIOP on Antisense Strand relative to SEQ ID NO: 2786 and are defined as follows: “+” > 1.0, “++” > 1.15, “+++” > 1.4, “++++” > 2.0. Levels of increased activity were determined for Activity FIOP on Sense Strand relative to SEQ ID NO: 2786 and are defined as follows: “+” > 1.0, “++” > 1.07, “+++” > 1.1, “++++” > 1.2.

While the invention has been described with reference to the specific embodiments, various changes can be made and equivalents can be substituted to adapt to a particular situation, material, composition of matter, process, process step or steps, thereby achieving benefits of the invention without departing from the scope of what is claimed.

For all purposes, each and every publication and patent document cited in this disclosure is incorporated herein by reference as if each such publication or document was specifically and individually indicated to be incorporated herein by reference. Citation of publications and patent documents is not intended as an indication that any such document is pertinent prior art, nor does it constitute an admission as to its contents or date.

1. An engineered RNA ligase, or a functional fragment thereof, comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to a reference sequence corresponding to residues 12 to 346 of an even-numbered SEQ ID NO. of SEQ ID NOs: 2-1470 and 1478-3058, or to a reference sequence corresponding to an even-numbered SEQ ID NO. of SEQ ID NOs: 2-1470 and 1478-3058, wherein the amino acid sequence comprises one or more substitutions relative to the reference sequence corresponding to residues 12 to 346 of SEQ ID NO: 2, 54, 244, 648, 862, 956, 1088, 1416, 1612, 1840, 1866, 1954, 2062, 2674, 2754, or 2786, or relative to the reference sequence corresponding to SEQ ID NO: 2, 54, 244, 648, 862, 956, 1088, 1416, 1612, 1840, 1866, 1954, 2062, 2674, 2754, or 2786.

2-4. (canceled)

5. The engineered RNA ligase of claim 1, comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12 to 346 of an even-numbered SEQ ID NO. of SEQ ID NOs: 4-1470 and 1478-3058, or to the reference sequence corresponding to an even-numbered SEQ ID NO. of SEQ ID NOs: 4-1470 and 1478-3058, wherein the amino acid sequence comprises one or more substitutions relative to the reference sequence corresponding to residues 12 to 346 of SEQ ID NO: 2, or relative to the reference sequence corresponding to SEQ ID NO: 2.

6. (canceled)

7. The engineered RNA ligase of claim 1, wherein the amino acid sequence of the engineered RNA ligase comprises at least a substitution at amino acid position 10, 11, 12, 13, 15, 17, 18, 21, 23, 25, 26, 28, 29, 30, 32, 33, 37, 38, 39, 40, 41, 42, 45, 52, 54, 56, 57, 58, 60, 61, 62, 64, 65, 67, 71, 73, 74, 75, 77, 80, 81, 83, 84, 86, 87, 89, 90, 91, 93, 95, 97, 99, 100, 101, 102, 103, 104, 105, 106, 107, 109, 113, 114, 116, 117, 119, 121, 122, 123, 125, 126, 127, 128, 129, 131, 135, 137, 140, 141, 143, 144, 148, 150, 154, 156, 157, 158, 159, 160, 161, 162, 165, 169, 170, 171, 173, 175, 177, 178, 179, 181, 183, 184, 185, 190, 191, 192, 193, 194, 197, 198, 204, 206, 208, 210, 212, 213, 214, 215, 218, 223, 224, 226, 228, 229, 230, 232, 236, 238, 241, 243, 247, 248, 249, 250, 251, 252, 253, 254, 255, 256, 257, 258, 260, 261, 264, 265, 266, 267, 268, 270, 271, 272, 274, 276, 277, 280, 282, 283, 286, 288, 289, 290, 291, 292, 294, 295, 296, 297, 298, 299, 300, 301, 302, 305, 306, 307, 310, 311, 315, 316, 317, 318, 323, 324, 325, 326, 327, 330, 331, 332, 334, 335, 336, 337, 338, 341, 342, 344, 345, or 346, or any combinations thereof, wherein the amino acid positions are relative to the reference sequence corresponding to residues 12 to 346 of SEQ ID NO: 2, or relative to the reference sequence corresponding to SEQ ID NO: 2.

8. The engineered RNA ligase of claim 1, wherein the amino acid sequence of the engineered RNA ligase comprises at least a substitution or amino acid residue 10F, 11G/N, 12A/F/L/M/P/R/T/V, 13W, 15M, 17P, 18A/H, 21S, 23K/T, 25A/D/F/G/L/M/S/T, 26L, 28A/L/M/V, 29D/S, 30A/L/T/V/W, 32E, 33A/I/L/T/V, 37A/F/K/L/M/R, 38A/E/L/R/S/T/V, 39P, 40D/I/P/S, 41E/I, 42T, 45S, 52C, 54L, 56I/L, 57V, 58M, 60A/C/D/R/S/V, 61A, 62A/V, 64A/Q/W, 65C, 67R, 71T, 73Q/W, 74D, 75A/D, 77M, 80E, 81Y, 83A/E/G/L/T/V, 84V, 86D/G/L, 87E, 89C/D/E/G/L/M/N/Q/R/S/T/V/W/Y, 90E/F, 91A/E/V, 93K/V, 95A/C/G/I, 97A/E, 991, 100A/G/H/L/R/S/T/V, 101P/V/W, 102T, 103G/V, 104T, 105K/L/R, 106G/L/M/Q/R/V/W, 107M/S, 109F/L, 113L/M, 114E, 116L, 117A/Q, 119L, 121N, 122C/F/G/L/Q/T/W, 123K/R, 125K/T, 126M, 127P, 128E, 129L/W, 131T, 135A/H/M/Q/V, 137N/R, 140I, 141D/G, 143A/C/E/F/H/M/S/T/V, 144L, 148F, 150D/L, 154S/V, 156G/H/Q/S, 157F/L/Q/V, 158R/S, 159F, 160R, 161M/V, 162C, 165M, 169G/N, 170H/Y, 171S, 173M, 175A, 177K, 178V, 179E, 181E/N/P/R/S, 183L/V, 184L, 185C/F/G/K/M/P/S/T/V, 190I/M, 191C, 192D/E/F/L/N, 193K/Q, 194A/G/V, 197W, 198M, 204W, 206L, 208H/I/K/L/P/T, 210G/V, 212E/G/I/K/L, 213D/N/R/T, 214A/C/H/K/L/Q/Y, 215G/T, 218F, 223C, 224A/Q/R, 226R, 228D/L, 229W, 230T/V, 232G/M/N/R/S, 236L/V, 238K/S/V, 241E/G/Q/R, 243H, 247I/L/R/S, 248F/G/I/K/T, 249K/L/R/S, 250E/I/S, 251C/E/F/G/L/R/S/T/Y, 252A/Q, 253L/W, 254E/K/L/N/Q/S/V, 255N/S/V/Y, 256A/E/G/M/S/W, 257D/E/G, 258D/M/P/V, 260G, 261D/P/T, 264A/E/G/S/V, 265A/F, 266C/I/L, 267I/M, 268F/G/M, 270L/M/V, 271A/E/G/T, 272M/S, 274A, 276A/G/M/V, 277A, 280L/N/W, 282A/M, 283C/V, 286D/E/F/M/V, 288E/L/R/W, 289A/D/E/I/P/T, 290D/H/N/P, 291H/L/Q/R/S/T/W, 292N/S, 294A/S/V/Y, 295C/F/G/K/L/M/P/Q/R/S/T/V/Y, 296E/G/L/Q/W, 297A/I/L, 298C/I/K/R/S/V, 299E/I/K/Q/R/T/V, 300A, 301A/F/I/L/M/V, 302A/C/I/L/S, 305V, 306G/L/T, 307K/R, 310H/L/M/N/Q/R/S/T, 311C, 315E/S, 316I/L/Y, 317E/F/G/Q/V/W, 318A/E/G/S, 323E/G, 324D/G/M, 325L, 326C/L, 327E/G/M/S, 330A/D/E/G/K/R, 331D/G/R/S/W, 332E/H/W, 334E/R, 335H/Q, 336I/L/M, 337A/V, 338G/L/M, 341A, 342R/S/T, 344K, 345Q/S, or 346S, or any combinations thereof, wherein the amino acid positions are relative to the reference sequence corresponding to residues 12 to 346 of SEQ ID NO: 2, or relative to the reference sequence corresponding to SEQ ID NO: 2.

9. The engineered RNA ligase of claim 1, wherein the amino acid sequence of the engineered RNA ligase comprises at least a substitution at amino acid position 10, 11, 12, 13, 17, 21, 23, 25, 26, 28, 29, 30, 32, 37, 38, 40, 41, 42, 45, 52, 54, 56, 57, 58, 60, 61, 62, 64, 65, 67, 71, 73, 74, 75, 77, 80, 83, 84, 87, 90, 91, 93, 97, 99, 100, 101, 102, 103, 104, 105, 106, 107, 109, 113, 114, 121, 122, 123, 125, 126, 127, 128, 129, 131, 135, 137, 140, 141, 143, 148, 150, 157, 158, 160, 161, 162, 165, 169, 170, 173, 178, 179, 184, 190, 191, 192, 193, 194, 197, 198, 204, 206, 208, 210, 213, 215, 218, 223, 228, 229, 232, 236, 238, 241, 243, 248, 249, 250, 251, 252, 253, 254, 255, 258, 261, 264, 265, 267, 268, 270, 271, 272, 274, 276, 277, 282, 286, 289, 290, 292, 294, 295, 297, 298, 299, 300, 301, 302, 305, 311, 318, 323, 324, 325, 327, 332, 336, 338, 341, 344, or 346; a substitution or amino acid residue 18A/H, 33A/I/T/V, 39P, 86D/L, 89C/D/E/L/N/Q/R/S/T/V/Y, 95A/C/G/I, 116L, 117A/Q, 119L, 144L, 154S/V, 156G/H/S, 171S, 175A, 177K, 181E/N/P/R/S, 183L, 212E/G/K/L, 224A/Q/R, 226R, 230T/V, 247I/L/R/S, 256E/G/M/S/W, 257D/E/G, 260G, 280L/W,283C/V, 288L/R/W, 291S, 296E/G/L/Q/W, 306G/T, 307K/R, 310H/L/M/N/Q/R/S/T, 315E, 316I/Y, 317E/F/G/Q/V/W, 326C/L, 330A/D/E/G/K, 331D/G/S, 334E, 335Q, 337A/V, 338G/L/M, 342R/S/T, or 345Q/S; or any combinations thereof, wherein the amino acid positions are relative to the reference sequence corresponding to residues 12 to 346 of SEQ ID NO: 2, or relative to the reference sequence corresponding to SEQ ID NO: 2.

10. (canceled)

11. The engineered RNA ligase of claim 1, wherein the amino acid sequence of the engineered RNA ligase comprises at least a substitution at amino acid position 11, 12, 23, 25, 29, 37, 38, 75, 86, 89, 93, 101, 122, 143, 157, 183, 192, 194, 198, 212, 214, 230, 241, 248, 249, 250, 251, 254, 266, 268, 280, 291, 295, 298, 307, 316, 318, 326, 330, or 331, or any combinations thereof, wherein the amino acid positions are relative to the reference sequence corresponding to residues 12 to 346 of SEQ ID NO: 2, or relative to the reference sequence corresponding to SEQ ID NO: 2.

12. (canceled)

13. (canceled)

14. The engineered RNA ligase of claim 1, wherein the amino acid sequence of the engineered RNA ligase comprises at least a substitution or substitution set at amino acid position(s) 256, 258, 254, 251, 248, 37, 54, 232, 15, 71, 26, 116, 38, 25, 106/251, 52, 33, 247, 37/249/266/316/330/334/335, 21, 11/37/249/266/316, 261, 11/266/316, 11/248/249/266, 331, 11/248/249, 255, 37/249/266/316/334, 266, 11/37/156/249/335, 37/156/248/249/266/334, 334, 156, 11/266/316/334, 11/39/156/248/316, 11/249, 37/156,107/251, 156/249/316, 37/156/316, 11/37/156/249, 11/37/334, 194, 37/316,175, 11/156/249/316, 11/37, 252, 12, 37/266, 11/37/156/266, 11/37/156/248/266, 37/236/248/249, 37/156/248/249, 11/266/330/335, 249, 11/156/248/249/316, 37/249/316, 11, 11/156, 11/37/249, 330, 316, 156/249/266/335, 249/266/316/330/335, 11/156/249, 266/316/330/334/335, 156/248/249/316/334, 37/156/248/249/316, 156/266, 248/249, 156/249/316/330/335, 218, 37/156/248/249/266, 11/37/156/248/249/316, 37/156/316/330, 11/37/248/249/316/330, 11/37/248/249, 249/330/335, 11/37/156/330/334, 40, 11/37/148/249/316, 83, 323, 193, 335, or 257, wherein the amino acid positions are relative to the reference sequence corresponding to residues 12 to 346 of SEQ ID NO: 2, or relative to the reference sequence corresponding to SEQ ID NO: 2.

15-17. (canceled)

18. The engineered RNA ligase of claim 1, comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12 to 346 of an even-numbered SEQ ID NO. of SEQ ID NOs: 4-1470 and 1478-3058, or to the reference sequence corresponding to an even-numbered SEQ ID NO. of SEQ ID NOs: 4-1470 and 1478-3058, wherein the amino acid sequence comprises one or more substitutions relative to the reference sequence corresponding to residues 12 to 346 of SEQ ID NO: 54, 244, 648, 862, 956, 1088, 1416, 1612, 1840, 1866, 1954, 2062, 2674, 2754, or 2786, or relative to the reference sequence corresponding to SEQ ID NO: 54, 244, 648, 862, 956, 1088, 1416, 1612, 1840, 1866, 1954, 2062, 2674, 2754, or 2786.

19. The engineered RNA ligase of claim 18, wherein the amino acid sequence of the engineered RNA ligase comprises at least a substitution at amino acid position 10, 11, 12, 13, 15, 17, 18, 21, 23, 25, 26, 28, 29, 30, 32, 33, 37, 38, 39, 40, 41, 42, 45, 52, 54, 56, 57, 58, 60, 61, 62, 64, 65, 67, 71, 73, 74, 75, 77, 80, 81, 83, 84, 86, 87, 89, 90, 91, 93, 95, 97, 99, 100, 101, 102, 103, 104, 105, 106, 107, 109, 113, 114, 116, 117, 119, 121, 122, 123, 125, 126, 127, 128, 129, 131, 135, 137, 140, 141, 143, 144, 148, 150, 154, 156, 157, 158, 159, 160, 161, 162, 165, 169, 170, 171, 173, 175, 177, 178, 179, 181, 183, 184, 185, 190, 191, 192, 193, 194, 197, 198, 204, 206, 208, 210, 212, 213, 214, 215, 218, 223, 224, 226, 228, 229, 230, 232, 236, 238, 241, 243, 247, 248, 249, 250, 251, 252, 253, 254, 255, 256, 257, 258, 260, 261, 264, 265, 266, 267, 268, 270, 271, 272, 274, 276, 277, 280, 282, 283, 286, 288, 289, 290, 291, 292, 294, 295, 296, 297, 298, 299, 300, 301, 302, 305, 306, 307, 310, 311, 315, 316, 317, 318, 323, 324, 325, 326, 327, 330, 331, 332, 334, 335, 336, 337, 338, 341, 342, 344, 345, or 346, or any combinations thereof, wherein the amino acid positions are relative to the reference sequence corresponding to residues 12 to 346 of SEQ ID NO: 54, 244, 648, 862, 956, 1088, 1416, 1612, 1840, 1866, 1954, 2062, 2674, 2754, or 2786, or relative to the reference sequence corresponding to SEQ ID NO: 54, 244, 648, 862, 956, 1088, 1416, 1612, 1840, 1866, 1954, 2062, 2674, 2754, or 2786.

20. (canceled)

21. The engineered RNA ligase of claim 18, wherein the amino acid sequence of the engineered RNA ligase comprises at least a substitution at amino acid position 10, 11, 12, 13, 17, 21, 23, 25, 26, 28, 29, 30, 32, 37, 38, 40, 41, 42, 45, 52, 54, 56, 57, 58, 60, 61, 62, 64, 65, 67, 71, 73, 74, 75, 77, 80, 83, 84, 87, 90, 91, 93, 97, 99, 100, 101, 102, 103, 104, 105, 106, 107, 109, 113, 114, 121, 122, 123, 125, 126, 127, 128, 129, 131, 135, 137, 140, 141, 143, 148, 150, 157, 158, 160, 161, 162, 165, 169, 170, 173, 178, 179, 184, 190, 191, 192, 193, 194, 197, 198, 204, 206, 208, 210, 213, 215, 218, 223, 228, 229, 232, 236, 238, 241, 243, 248, 249, 250, 251, 252, 253, 254, 255, 258, 261, 264, 265, 267, 268, 270, 271, 272, 274, 276, 277, 282, 286, 289, 290, 292, 294, 295, 297, 298, 299, 300, 301, 302, 305, 311, 318, 323, 324, 325, 327, 332, 336, 338, 341, 344, or 346; a substitution or amino acid residue 18A/H, 33A/I/T/V, 39P, 86D/L, 89C/D/E/L/N/Q/R/S/T/V/Y, 95A/C/G/I, 116L, 117A/Q, 119L, 144L, 154S/V, 156G/H/S, 171S, 175A, 177K, 181E/N/P/R/S, 183L, 212E/G/K/L, 224A/Q/R, 226R, 230T/V, 247I/L/R/S, 256E/G/M/S/W, 257D/E/G, 260G, 280L/W, 283C/V, 288L/R/W, 291S, 296E/G/L/Q/W, 306G/T, 307K/R, 310H/L/M/N/Q/R/S/T, 315E, 316I/Y, 317E/F/G/Q/V/W, 326C/L, 330A/D/E/G/K, 331D/G/S, 334E, 335Q, 337A/V, 338G/L/M, 342R/S/T, or 345Q/S; or any combinations thereof, wherein the amino acid positions are relative to the reference sequence corresponding to residues 12 to 346 of SEQ ID NO: 54, 244, 648, 862, 956, 1088, 1416, 1612, 1840, 1866, 1954, 2062, 2674, 2754, or 2786, or relative to the reference sequence corresponding to SEQ ID NO: 54, 244, 648, 862, 956, 1088, 1416, 1612, 1840, 1866, 1954, 2062, 2674, 2754, or 2786.

22. (canceled)

23. The engineered RNA ligase of claim 18, wherein the amino acid sequence of the engineered RNA ligase comprises at least a substitution at amino acid position 11, 12, 23, 25, 29, 37, 38, 75, 86, 89, 93, 101, 122, 143, 157, 183, 192, 194, 198, 212, 214, 230, 241, 248, 249, 250, 251, 254, 266, 268, 280, 291, 295, 298, 307, 316, 318, 326, 330, or 331, or any combinations thereof, wherein the amino acid positions are relative to the reference sequence corresponding to residues 12 to 346 of SEQ ID NO: 54, 244, 648, 862, 956, 1088, 1416, 1612, 1840, 1866, 1954, 2062, 2674, 2754, or 2786, or relative to the reference sequence corresponding to SEQ ID NO: 54, 244, 648, 862, 956, 1088, 1416, 1612, 1840, 1866, 1954, 2062, 2674, 2754, or 2786.

24-27. (canceled)

28. The engineered RNA ligase of claim 18, wherein the amino acid sequence of the engineered RNA ligase comprises a substitution or substitution set at amino acid positions(s) 156/232/334/335, 12/254/256, 12/230, 12/230/251, 12/230/251/254/256, 12/38/230, 230/251, 12/38/254, 230/256, 254, 230, 12/230/254, 12/38/230/251/256, 12/251, 12/251/254/256, 38, 12/230/256, 12, 12/25/38/230/251, 12/38/102/230/251/256, 12/256, 38/230/256, 256, 12/230/251/256, 230/251/254, 230/254, 251/254, 12/25/230, 38/230, 38/251, 12/38/230/256, 102, 251, 12/25/251/256, 12/102/256, 12/38/230/251/254/256, 12/251/256, 102/230/251/254/256, 12/38/251, 12/230/251/254, 156/248/334/335, 12/25/38, 12/102/230/251/254, 12/38/251/254, 12/251/254, 12/102, 38/230/251/254/256, 12/25/230/251/256, 12/230/311, 12/254, 38/230/251/254, 12/38/251/256, 156/334/335, 257/258/334, 12/25/38/251/256, 334, 12/25/230/254/256, 38/251/256, 12/38/230/251/254, 12/38, 257/258/334/335, 12/38/254/256, 232/248/257/258/334/335, 156/258/334/335, 156/249/334/335, 12/102/230/254, 25, 156/257/258/334/335, 12/102/230, 156/248/257/258/334, 248/249/334/335, 232/257/258/334/335, 334/335, 156/334, 12/38/102/256, 156/257/334/335, 12/140/251/254, 251/254/256, 248/334/335, 156/258/334, 156/248/330/335, 12/38/256, 232/334/335, 156/249/334, 330/334/335, 156/248/334, 156/335, 257/334, 248/334, 232/248/334/335, 12/25, 1938/230/251/256, 156/248/249/334/335, 330/335, 248/257/334/335, 232/248/334, 156/232/248/334/335, 232/257/330/334, 156/248/291/334/335, 156/232/248/257/258/334, 156/232/249/334/335, 12/25, 156/248/330/334/335, 156/248/249/334, 232/248/330/335, 156/232/330/334/335, 197, 338, 327, 192, 331, 326, 345, 101, 100, 141, 198, 64, 306, 317, 265, 296, 194, 62, 90, 307, 103, 210, 105, or 61, wherein the amino acid positions are relative to the reference sequence corresponding to residues 12 to 346 of SEQ ID NO: 54, or relative to the reference sequence corresponding to SEQ ID NO: 54.

29. (canceled)

30. (canceled)

31. The engineered RNA ligase of claim 18, wherein the amino acid sequence of the engineered RNA ligase comprises at least a substitution or substitution set at amino acid position(s) 141/197/326/327, 100/101/338, 141/198/326, 64/100/101, 100/338, 101/141/198/326, 101/198/326/327, 198/326, 64/100/101/338/345, 197/326, 198/327, 141/198/327, 198/326/327, 197/326/327, 64/100/338, 64/100/101/156/197/338, 64/100/101/338, 100/197/338, 197/338, 101/197/326, 64/100/197/338, 101/198, 141/197/327, 64/100/101/197/338, 101/326, 101/197/326/327, 197/327, 197, 141/197, 101, 101/198/326, 64/197/198/338, 141, 101/141, 100/101/197/338, 141/326, 338, 326, 197/198/338, 64/101/338, 64/197/198, 101/197, 100/101, 100/109/197/338, 101/197/338, 197/198, 141/326/327, 100/197, 64/100, 64/197/338, 100/197/198, 101/338, 141/327, 101/306, 64/100/101/197, 101/326/327, 100, 327, 326/345, 64/100/101/197/198, 326/327, 64/101,100/101/197, 101/327, 64/100/197/198/338, 306, 64/338, 101/141/326/327, 64/101/197/338, or 101/198/306/327, wherein the amino acid positions are relative to the reference sequence corresponding to residues 12 to 346 of SEQ ID NO: 244, or relative to the reference sequence corresponding to SEQ ID NO: 244.

32. (canceled)

33. (canceled)

34. The engineered RNA ligase of claim 18, wherein the amino acid sequence of the engineered RNA ligase comprises at least a substitution or substitution set at amino acid position(s) 194/330/331, 298, 331, 192/193/194/298, 330, 192/330/331, 192/193/194/331, 298/330/331, 194/298/330/331, 192/193/330/331, 192/298/331, 192/193, 194/330, 60/100/156, 193/296/331, 298/331, 193/194/330/331, 194, 194/298/331, 193/298, 193/330, 192, 192/193/194/298/331, 192/193/194/298/330, 192/193/298/331, 100/156, 62/100,298/330, 60/100,192/193/298/330/331, 100, 193/194/298, 338, 62, 247, 18, 37, 254, 33, 261, 251, 260, 12, 117, 256, 255, 38, 29, 25, or 252, wherein the amino acid positions are relative to the reference sequence corresponding to residues 12 to 346 of SEQ ID NO: 648, or relative to the reference sequence corresponding to SEQ ID NO: 648.

35. (canceled)

36. (canceled)

37. The engineered RNA ligase of claim 18, wherein the amino acid sequence of the engineered RNA ligase comprises a substitution or substitution set at amino acid positions(s) 254, 18/254,247/317, 37/317/342, 33, 18/156/254, 18, 156, 254/338, 254/307, 33/37/317, 37, 37/156/317, 156/265/317, 265/317, 156/254, 33/156/317, 317, 156/317, 37/317, 18/338,338, 156/247/317, 33/317, 37/247/265/317, 33/37/156/317, 156/247/296/317, 18/307,156/296/317, 156/247, 37/247/317, 18/254/307, 307, 265, 33/37, 18/156, 33/156/296/317, 18/156/254/338, 37/265/317, 33/37/156/247/317, 296, 33/296/317, 156/296, 247/296/317, 37/296/317, 247/296, 33/37/156, 296/317, 156/254/307, 18/156/307, or 33/37/265, wherein the amino acid positions are relative to the reference sequence corresponding to residues 12 to 346 of SEQ ID NO: 862, or relative to the reference sequence corresponding to SEQ ID NO: 862.

38. (canceled)

39. (canceled)

40. The engineered RNA ligase of claim 18, wherein the amino acid sequence of the engineered RNA ligase comprises at least
(a) a substitution or substitution set at amino acid position(s) 12/25/29/156, 12/255/256/257, 12/156/255, 247/252, 12/25, 12, 12/25/29/156/255, 256, 12/156, 12/25/156/255/256/257/261, 18/247/251, 25/255/256/257, 12/25/257, 255/257, 12/25/29/255/257, 256/257, 12/255/257, 12/156/255/256/257, 25, 18/38/251/252, 12/256/257/261, 12/29, 12/25/255/256/257/261, 38, 12/25/29/256/257, 29/257, 12/261,251, 249/251, 12/255, 12/29/156/256/257, 252, 255/257/261, 18, 29/255/257, 255/256/257, 12/25/29/156/256/257, 25/255/256/257/261, 29/255/256/257/261, 25/256, 18/38/247/252/327, 18/247, 25/29, 12/25/156/255/257, 18/156/251/252, 12/255/261, 25/255, 12/255/256/261, 156/255/257, 156/257, 12/156/256/257, 25/156/256/257, 12/25/29/156/257/261, 25/261, 12/156/255/256/261, 12/25/29/255/256/257, 38/252,255, 249/252, 12/156/255/256, 12/25/29/261, 257, 12/255/256, 12/29/255, 12/255/257/261, 12/156/257, 192/249/251/252, 12/25/29/255/261, 12/156/255/257, 12/29/255/257, 12/29/256/257, 18/252,249, 156/250, 38/251/252, 156/255/256/257, 25/257, 12/255/256/257/261, 25/156/261, 156/252, 12/25/255/256/257, 12/25/29/156/255/257, 29/156,251/252, 256/257/261, 25/156/255/256/257, 156/249/251/252, 25/29/255, 12/29/156, 255/256, 38/250/252, 247/249/252, 12/256/257, 60, 12/256, 12/156/256, 25/255/257/261, 12/25/29/255/256/261, 12/29/156/255/256, 12/25/156/255/256, 247/251/252, 256/261, 38/251, 12/25/29/156/261, 12/257,156/255/256/261, 12/25/156/255, 12/25/29/156/255/257/261, 12/29/256/261, 29/156/261, 25/29/156/257/261, 12/25/255/256/261, or 12/156/255/261, wherein the amino acid positions are relative to the reference sequence corresponding to residues 12 to 346 of SEQ ID NO: 956, or relative to the reference sequence corresponding to SEQ ID NO: 956; or
(b) a substitution at amino acid position 157, 183, 95, 185, 181, 213, 89, 28, 190, 214, or 143, or any combinations thereof, wherein the amino acid positions are relative to the reference sequence corresponding to residues 12 to 346 of SEQ ID NO: 956, or relative to the reference sequence corresponding to SEQ ID NO: 956.

41-43. (canceled)

44. The engineered RNA ligase of claim 18, wherein the amino acid sequence of the engineered RNA ligase comprises at least a substitution or substitution set at amino acid position(s) 95/157, 95/183/185/214, 89/183, 28/89/143/183, 28/143/157, 181/183, 143/181/183/214, 143/181/183, 89/143/183, 28/143/181/183, 143/157, 95/181/183, 157, 143/183, 89/143/157/183, 89/95/157, 89/157, 89/95/143/181, 95/143, 89/157/183/214, 143/214, 143/181/183/185, 28/95/143, 143/183/214, 28/95/183/214, 89/143/157, 28/95, 95/143/183/185, or 95/143/157/183, wherein the amino acid positions are relative to the reference sequence corresponding to residues 12 to 346 of SEQ ID NO: 1088, or relative to the reference sequence corresponding to SEQ ID NO: 1088.

45. (canceled)

46. (canceled)

47. The engineered RNA ligase of claim 18, wherein the amino acid sequence of the engineered RNA ligase comprises at least
(a) a substitution or substitution set at amino acid position(s) 83/137/150, 137/213, 137, 150/190, 143/150, 104/150, 60/143/150/190, 60/150/190/213, 104/137/154, 123/137, 137/150/154/190, 123/143/150, 60/104/143/150/213, 60/95/143/190, 123/137/190, 60/123,104/123, or 60/123/143, wherein the amino acid positions are relative to the reference sequence corresponding to residues 12 to 346 of SEQ ID NO: 1416, or relative to the reference sequence corresponding to SEQ ID NO: 1416; or
(b) a substitution or substitution set at amino acid position(s) 38, 249, 255, 23, 33, 25, 226, 29, 37, 10, 250, 75, 230, 77, 251, 228, 81, 224, 64, 254, 268, 65, 232, 332, 331, 330, 101, 307, 137, 223, 280, 289, 288, 342, 93, 296, 86, 241, 126, 277, 87, 247, 243, 84, 83, 192, 105, 25/29, 25/29/250, 25/29/75/77/250/255, 25/75/230, 25/77/226/255, 25/230,250/255, 29/75/226/230/255, 29/75/77, 29/75/77/226, 29/75/77/230/255, 29/226, 29/226/230, 29/230/255, 33/228, 33/249, 33/37/228, 33/37/38/228, 33/37/38/228/249, 33/38, 75/250/255, 75/226, 75/226/250/255, 75/226/230/250/255, 75/77, 75/77/255, 75/77/250/255, 75/77/226/255, 75/77/226/250/255, 75/77/230, 75/77/230/250, 75/230, 75/230/255, 75/230/250, 226/255, 226/250, 226/230, 226/230/255, 37/228, 37/228/249, 37/38, 37/38/228, 37/38/228/249, 37/38/249, 38/249/291, 10/228, 10/228/249, 10/33/249, 10/33/37, 10/33/37/38/249, 10/33/38/249, 10/33/38, 10/33/249/251, 10/33/37/228, 10/33/37/249, 10/33/37/38/228/249, 10/33/37/38, 10/249, 10/37, 10/37/228, 10/37/228/249, 10/37/249, 10/37/38/228, 10/37/38/228/249, 10/37/38, 10/38/249, 10/38, 10/38/228, 77/255, 77/250, 77/250/255, 77/226, 77/226/255, 77/230, 77/230/255, 230/255, 230/250, 230/250/255, 23/25/226/255, 23/25/226/230, or 23/25/230, wherein the amino acid positions are relative to the reference sequence corresponding to residues 12 to 346 of SEQ ID NO: 1416, or relative to the reference sequence corresponding to SEQ ID NO: 1416; or
(c) a substitution or substitution set at amino acid position(s) 86, 255, 306, 250, 75, 318, 335, 241, 248, 344, 38, 272, 83, 257, 258, or 323, wherein the amino acid positions are relative to the reference sequence corresponding to residues 12 to 346 of SEQ ID NO: 1416, or relative to the reference sequence corresponding to SEQ ID NO: 1416.

48-51. (canceled)

52. The engineered RNA ligase of claim 18, wherein the amino acid sequence of the engineered RNA ligase comprises at least a substitution or substitution set at amino acid position(s) 101, 291, 291/307, 331, 33/75, 33/75/249/291/331, 33/38, 33/93/249, 38/75/291/307, 38/75/249/250, 38/75/93/271, 38/93, 10/75, 10/38/75, 10/38/75/291, 10/38/75/249, 10/38/75/93, 64/224,224, 23/101/307, 23/268, 23/37/64/224/254, 23/64/81, 23/224/268, 23/224/254, 23, 23/64/224, 64, 75/249/307, 93, 33/38/75/249, 75/249, 25/38/75/93/291, 33/38/75, 75, or 23/254, wherein the amino acid positions are relative to the reference sequence corresponding to residues 12 to 346 of SEQ ID NO: 1612, or relative to the reference sequence corresponding to SEQ ID NO: 1612.

53. (canceled)

54. (canceled)

55. The engineered RNA ligase of claim 18, wherein the amino acid sequence of the engineered RNA ligase comprises at least a substitution or substitution set at amino acid position(s) 38/65/192, 38/65, 38/65/224, 38/64, 93/307, 38, 38/224, 38/192/224, 65, 192/224, 75/307, 75/250/268/307, 250/268/307/342, 224, 65/192,268, 64/192,250/268/291/342, 75/93/249/307/342, 250/291, 38/64/192, 38/64/224, 93/249/307, 38/64/86/192/241, 38/342,248/250/307, 75/77/250/268/307/342, 93/268, 192, 38/192, or 64, wherein the amino acid positions are relative to the reference sequence corresponding to residues 12 to 346 of SEQ ID NO: 1840, or relative to the reference sequence corresponding to SEQ ID NO:
1840.

56. (canceled)

57. (canceled)

58. The engineered RNA ligase of claim 18, wherein the amino acid sequence of the engineered RNA ligase comprises at least
(a) a substitution or substitution set at amino acid position(s) 38/248/251, 38/248/250/251/277, 248/250/251, 38/93/192/248/251, 38/192/248/251, 250/251, 38/248/250/251, 248/251/344, 192/248/250/251, 38/248/250, 38/93/250/251/277, 38/255/280/318, 248, 38/86/241/255/280/318/335, 38/255, 38, 318, 38/101/255/280, 38/318/335, 38/255/318, 192/250/251/344, 38/335,318/335, 38/277, 38/93/251, 192/251, 255, 38/64/318, 38/64/241/255/318, 251, 64/101/255/280/318, 38/101/255/280/318/335, 38/255/280, 38/251/277, 64/318, 38/64/255/318, 241/255/280/318, 38/86/101/241/318, 38/241, 38/255/318/335, 38/64/101/318, 38/64/255/280/318, 241/255/318, or 38/241/335, wherein the amino acid positions are relative to the reference sequence corresponding to residues 12 to 346 of SEQ ID NO: 1866, or relative to the reference sequence corresponding to SEQ ID NO: 1866; or
(b) a substitution or substitution set at amino acid position(s) 89, 122, 181, 28, 125, 157, 238, 330, 212, 143, 208, 290, 161, 214, 84, 270, 91, 295, 316, 286, 301, 170, 264, 289, 190, 183, 128, 60, 310, 131, 302, 267, 288, 185, 291, 129, 280, 298, 158/215, 28/122,276, 274, 271, 215, 294, 106, 58, 135, 45, 299, 315, 204, 332, 169, 305, 154, 158, 297, 95, 337, 324, 150, 179, 283, 206, 236, 266, 213, 336, 178, 99, 184, 282, 57, 177, 109, 191, 107, 160, 144, 56, 159, 341, 325, or 91, wherein the amino acid positions are relative to the reference sequence corresponding to residues 12 to 346 of SEQ ID NO: 1866, or relative to the reference sequence corresponding to SEQ ID NO: 1866.

59-61. (canceled)

62. The engineered RNA ligase of claim 18, wherein the amino acid sequence of the engineered RNA ligase comprises at least a substitution or substitution set at amino acid position(s) 93/232/291/306, 93/232/280/306, 93/241/257/306/335, 232/280, 93/291/306, 93/257, 93/306, 93/280,192/318, 93/280/291, 93/241/291/306, 192, 257/291, 241/257/306, 280/335, 93/291/306/335, 93/335, 93/232/335, 64/318, 93/241, 93/232/241/280, 64/192/318/323, 232/280/306, 93/232,291/335, 257/258, 93/232/241/257/258/280/335, 318/323, 93/241/291/335, 83/86/323, 257/258/291, 101/224/318, 83/192/255/296, 171/232/257/258/280, 83/224/255/318, 64/192/250/318, 101/296, 232/241/280, 64/255/318, 86/250/255, 83/86/101, 83/86, 93/241/258/335, 250/296/323, or 257/258/280, wherein the amino acid positions are relative to the reference sequence corresponding to residues 12 to 346 of SEQ ID NO: 1954, or relative to the reference sequence corresponding to SEQ ID NO: 1954.

63. (canceled)

64. (canceled)

65. The engineered RNA ligase of claim 18, wherein the amino acid sequence of the engineered RNA ligase comprises at least a substitution or substitution set at amino acid position(s) 83/84/330, 91/270/290, 83/181,161/290/330, 181/330, 161/270/330, 270/290, 83/84/270/318/330, 64/83/84/161/192/290/318, 91/161/290/318/330, 161/330, 161/214/270/290/330, 192/270/318, 91/161/192/330, 161/270, 181/270/330, 91/181/192/290, 161/290, 83/161/270, 83/192/318, 214/330, 83/161/290/330, 64/83/161/290/330, 83/161/192/214/318, 122/143/212/295, 161/192/270/290, 181/290, 91/161/214/270/290/318, 91/192/214/270, 214/290, 64/83/161/270/318/330, 91/161/181/330, 161/318/330, 91/270/290/330, 64/181/290/318, 181/270/290/318, 64/83/161/192/270/290/318/330, 64/83/161/214/290/318, 122/192/212, 64/83/91/192/214/330, 83/91/161/181/290/318, 91/161/192/214/270/290, 91/161/181/290/330, 83/91/125/161/270, 28/212,122/212, 28/64/83/122/143/192/212/295/318, 122/295, 91/161/192/318, 28/122/143/212, 64/161/290, 64/122/208/318, 64/83/192/290/318, 64/83/161/290, 181/192/318, 83/161/192/318, 214/270, 83/122, or 192/212, 28/122, wherein the amino acid positions are relative to the reference sequence corresponding to residues 12 to 346 of SEQ ID NO: 2062, or relative to the reference sequence corresponding to SEQ ID NO: 2062.

66. (canceled)

67. (canceled)

68. The engineered RNA ligase of claim 18, wherein the amino acid sequence of the engineered RNA ligase comprises at least
(a) a substitution or substitution set at amino acid position(s) 28/291/295/298/315, 28/95/291/295/298/315, 143/295/298/315, 291/295/298, 28/143/295/298, 28/291/295/298, 143/185/291/295/298, 28/143/185/291/298/336, 28/298,280/295, 143/298/336, 143/291/295/298, 291/295/298/315/336, 143/295/298, 28/143/295/298/336, 95/143/295/298, 143/280/290, or 143/291/298, wherein the amino acid positions are relative to the reference sequence corresponding to residues 12 to 346 of SEQ ID NO: 2674, or relative to the reference sequence corresponding to SEQ ID NO: 2674; or
(b) a substitution or substitution set at amino acid position(s) 208, 25, 90, 141, 258, 248, 318, 117, 299, 33, 80, 253, 83, 254, 256, 30, 327, 73, 100, 97, 119, 265, 296, 32, 156, 40, 105, 107, 41/114, 41, or 64, wherein the amino acid positions are relative to the reference sequence corresponding to residues 12 to 346 of SEQ ID NO: 2674, or relative to the reference sequence corresponding to SEQ ID NO: 2674; or
(c) a substitution or substitution set at amino acid position(s) 208, 25, 90, 141, 258, 248, 318, 117, 299, 33, 80, 253, 83, 254, 256, 30, 327, 73, 100, 97, 119, 265, 296, 32, 156, 40, 105, 107, 41/114, 41, or 64, wherein the amino acid positions are relative to the reference sequence corresponding to residues 12 to 346 of SEQ ID NO: 2674, or relative to the reference sequence corresponding to SEQ ID NO: 2674.

69-72. (canceled)

73. The engineered RNA ligase of claim 18, wherein the amino acid sequence of the engineered RNA ligase comprises at least a substitution or substitution set at amino acid position(s) 106/264/280, 280/330, 28/106/264/291, 28/264/280/330, 57/60/135/264/291, 28/135/264/280, 106/264/280/291, 28/106/264/280, 280/299, 28/57/89/106/264/280/291, 264, 185/291, 28/106/299, 28/214/264/299, 264/330, 28/89/214/291, 28/185/271/310, 122/271, or 89/106/135/264/280/330, wherein the amino acid positions are relative to the reference sequence corresponding to residues 12 to 346 of SEQ ID NO: 2754, or relative to the reference sequence corresponding to SEQ ID NO: 2754.

74. (canceled)

75. (canceled)

76. The engineered RNA ligase of claim 18, wherein the amino acid sequence of the engineered RNA ligase comprises at least a substitution or substitution set at amino acid position(s) 25/228/248/264, 32/113/228/248, 25/32/113/241/248/264/299, 25/113/248/264/318, 25/127/228/264/299/318, 25/113/228/248/327, 228/241/264, 264/299/318/327, 25/113/241/248/264/318, 25/127/248/299/318, 113/127/228/264/299, 25/127, 25/228/248/264/327, 25/113/264/299, 32/113/264/318, 25/32/241/264/327, 113/264/327, 25/127/228/248/318, 327, 32/113/254/264, 25/228/241/264, 25/113/228/241/318, or 113/327, wherein the amino acid positions are relative to the reference sequence corresponding to residues 12 to 346 of SEQ ID NO: 2786, or relative to the reference sequence corresponding to SEQ ID NO: 2786.

77. The engineered RNA ligase of claim 1, wherein the RNA ligase comprises an amino acid sequence comprising residues 12 to 346 of an even numbered SEQ ID NO. of SEQ ID NOs: 4-1470 and 1478-3058, or an amino acid sequence comprising an even numbered SEQ ID NO. of SEQ ID NOs: 4-1470 and 1478-3058.

78. An engineered RNA ligase comprising an amino acid sequence comprising
(a) in the N-terminal domain one or more amino acid residues 12A/F/L/M/P/R/T/V, 13W, 15M, 17P, 18A/H, 21S, 23K/T, 25A/D/F/G/L/M/S/T, 26L, 28A/L/M/V, 29D/S, 30A/L/T/V/W, 32E, 33A/I/L/T/V, 37A/F/K/L/M/R, 38A/E/L/R/S/T/V, 39P, 40D/I/P/S, 41E/I, 42T, 45S, 52C, 54L, 56I/L, 57V, 58M, 60A/C/D/R/S/V, 61A, 62A/V, 64A/Q/W, 65C, 67R, 71T, 73Q/W, 74D, 75A/D, 77M, 80E, 81Y, 83A/E/G/L/T/V, 84V, 86D/G/L, 87E, 89C/D/E/G/L/M/N/Q/R/S/T/V/W/Y, 90E/F, 91A/E/V, 93K/V, 95A/C/G/I, 97A/E, 991, 100A/G/H/L/R/S/T/V, 101P/V/W, 102T, 103G/V, 104T, 105K/L/R, 106G/L/M/Q/R/V/W, 107M/S, 109F/L, 113L/M, 114E, 116L, 117A/Q, 119L, 121N, 122C/F/G/L/Q/T/W, 123K/R, 125K/T, 126M, 127P, 128E, 129L/W, 131T, 135A/H/M/Q/V, 137N/R, 140I, 141D/G, 143A/C/E/F/H/M/S/T/V, 144L, 148F, 150D/L, 154S/V, 156G/H/Q/S, 157F/L/Q/V, 158R/S, 159F, 160R, 161M/V, 162C, 165M, 169G/N, 170H/Y, 171S, 173M, 175A, 177K, 178V, 179E, 181E/N/P/R/S, 183L/V, 184L, 185C/F/G/K/M/P/S/T/V, 190I/M, 191C, 192D/E/F/L/N, 193K/Q, 194A/G/V, 197W, 198M, 204W, 206L, 208H/I/K/L/P/T, 210G/V, 212E/G/I/K/L, 213D/N/R/T, 214A/C/H/K/L/Q/Y, 215G/T, 218F, 223C, 224A/Q/R, 226R, 228D/L, 229W, 230T/V, 232G/M/N/R/S, 236L/V, 238K/S/V, 241E/G/Q/R, and 243H, or at equivalent positions thereof;
(b) in the C-terminal domain one or more amino acid residues 256A/E/G/M/S/W, 257D/E/G, 258D/M/P/V, 260G, 261D/P/T, 264A/E/G/S/V, 265A/F, 266C/I/L, 267I/M, 268F/G/M, 270L/M/V, 271A/E/G/T, 272M/S, 274A, 276A/G/M/V, 277A, 280L/N/W, 282A/M, 283C/V, 286D/E/F/M/V, 288E/L/R/W, 289A/D/E/I/P/T, 290D/H/N/P, 291H/L/Q/R/S/T/W, 292N/S, 294A/S/V/Y, 295C/F/G/K/L/M/P/Q/R/S/T/V/Y, 296E/G/L/Q/W, 297A/I/L, 298C/I/K/R/S/V, 299E/I/K/Q/R/T/V, 300A, 301A/F/I/L/M/V, 302A/C/I/L/S, 305V, 306G/L/T, 307K/R, 310H/L/M/N/Q/R/S/T, 311C, 315E/S, 316I/L/Y, 317E/F/G/Q/V/W, 318A/E/G/S, 323E/G, 324D/G/M, 325L, 326C/L, 327E/G/M/S, 330A/D/E/G/K/R, 331D/G/R/S/W, 332E/H/W, 334E/R, 335H/Q, 336I/L/M, 337A/V, 338G/L/M, 341A, 342R/S/T, 344K, 345Q/S, and 346S; or at equivalent positions thereof; and/or
(c) in the linker region connecting the N-terminal and C-terminal domain one or more amino acid residues 247I/L/R/S, 248F/G/I/K/T, 249K/L/R/S, 250E/I/S, 251C/E/F/G/L/R/S/T/Y, 252A/Q, 253L/W, 254E/K/L/N/Q/S/V, and 255N/S/V/Y, or at equivalent positions thereof.

79-85. (canceled)

86. The engineered RNA ligase of claim 1, wherein the engineered RNA ligase has RNA ligase 2 activity and has at least one improved property as compared to a reference RNA ligase, wherein the improved property is selected from i) increased activity, ii) increased stability, iii) increased thermostability, iv) increased product yield, v) increased activity on polynucleotides ligase substrates with phosphorothioate internucleoside linkages, vi) increased activity on oligonucleotides with 2′-modifications, vii) increased substrate tolerance, or any combination of i), ii), iii), iv), v), vi), and vii), and wherein the reference RNA ligase has the sequence corresponding to residues 12 to 346 of SEQ ID NO: 2, 54, 244, 648, 862, 956, 1088, 1416, 1612, 1840, 1866, 1954, 2062, 2674, 2754, or 2786, or the sequence corresponding to SEQ ID NO: 2, 54, 244, 648, 862, 956, 1088, 1416, 1612, 1840, 1866, 1954, 2062, 2674, 2754, or 2786.

87-91. (canceled)

92. A recombinant polynucleotide comprising a polynucleotide sequence encoding an engineered RNA ligase of claim 1.

93-96. (canceled)

97. An expression vector comprising a recombinant polynucleotide of claim 92.

98. (canceled)

99. (canceled)

100. A host cell comprising an expression vector of claim 97.

101. (canceled)

102. (canceled)

103. A method of producing an engineered RNA ligase polypeptide in a host cell comprising culturing a host cell of claim 100, under suitable culture conditions such that the engineered RNA ligase is produced.

104. (canceled)

105. (canceled)

106. A composition comprising an RNA ligase of claim 1.

107. (canceled)

108. A method of ligating at least a first polynucleotide strand and a second polynucleotide strand, comprising contacting a first polynucleotide strand and a second polynucleotide strand with an engineered RNA ligase of claim 1 in presence of a nucleotide substrate under conditions suitable for ligation of the first polynucleotide strand to the second polynucleotide strand, wherein the first polynucleotide strand comprises a ligatable 5′-end and the second polynucleotide strand comprises a 3′-end ligatable to the 5′-end of the first polynucleotide.

109-126. (canceled)
